(19) United States
(12) Patent Application Publication
(10) Pub. No.: US 2023/0302155 A1
(43) Pub. Date: Sep. 28, 2023

(54) COMPOSITIONS AND METHODS FOR IN VIVO GENERATION OF CAR EXPRESSING CELLS

(71) Applicant: Novartis AG, Basel (CH)

(72) Inventors: Sandeep Tharian Koshy, Cambridge, MA (US); Glenn Dranoff, Sudbury, MA (US); Maria Anna Sofia Broggi, Cambridge, MA (US); Chris Bridgeman, Bedford, MA (US); Stephen Michael Canham, Cambridge, MA (US); Yoel Melles, Cambridge, MA (US); Regis Cebe, Basel (CH); Brian Walter Granda, Salisbury, MA (US); Louise Mary Treanor, Cambridge, MA (US); Shyamali Jayashankar, Cambridge, MA (US); Jennifer Yang, Somerville, MA (US); Amy Rayo, Cambridge, MA (US); Andrew Patrick Price, Cambridge, MA (US); Darko Skegro, Riehen (CH); Justine Guyot, Zimmersheim (FR); Tushar Dattu Apsunde, Cambridge, MA (US); Cameron Chuck-munn Lee, Cambridge, MA (US); Michael Bardroff, Loerrach (DE); Sandra Miller, Munich (DE)

(21) Appl. No.: 18/022,058

(22) PCT Filed: Aug. 20, 2021
(86) PCT No.: PCT/US2021/046994

§ 371 Date: Feb. 17, 2023

Related U.S. Application Data
(60) Provisional application No. 63/154,609, filed on Feb. 26, 2021.
(60) Provisional application No. 63/068,876, filed on Aug. 21, 2020.

(51) Int. Cl. A61K 47/69; A61K 9/06; A61K 38/18; C12N 15/86; A61K 38/20; A61K 38/17; C07K 14/705; C07K 14/725; C07K 16/28; A61K 39/395; A61K 39/00; A61K 31/4745

(52) U.S. Cl. CPC A61K 47/6927; A61K 9/06; A61K 38/1858; C12N 15/86; A61K 38/2086; A61K 38/2046; A61K 47/6901; A61K 38/1774; C07K 14/70517; C07K 14/70578; A61K 38/177; C07K 14/70521; C07K 14/7051; C07K 16/2806; C07K 16/2818; C07K 16/2809; A61K 39/3955; A61K 39/4631; A61K 31/4745; C12N 2740/15043; C12N 2740/15071; A61K 2039/505; C07K 2319/33; C07K 2319/03; A61K 2239/21; C07K 2319/02; A61K 2239/22; C07K 2317/31; A61K 2239/29; C07K 2317/55; C07K 2317/622; C07K 2317/524; C07K 2317/526; C07K 2317/522; A61K 2039/545; A61K 2239/38; A61K 2239/13; C07K 2317/75

(57) ABSTRACT

Aspects of this disclosure relate generally to the use of biomaterials for the in vivo generation of CAR expressing cells. In some embodiments, the biomaterials comprise one or more of a cell recruitment composition, a viral vector, and/or a cell activation agent.

### RELATED APPLICATIONS

This application claims priority to U.S. Provisional Application 63/068,876 filed on Aug. 21, 2020, and U.S. Provisional Application 63/154,609 filed on Feb. 26, 2021, the entire contents of which are hereby incorporated by reference.

### SEQUENCE LISTING

The instant application contains a Sequence Listing which has been submitted electronically in ASCII format and is hereby incorporated by reference in its entirety. Said ASCII copy, created on Aug. 20, 2021, is named N2067-7176WO_SL.txt and is 987,095 bytes in size.

### FIELD OF THE INVENTION

Aspects of this disclosure relate generally to the use of biomaterials for the in vivo generation of CAR expressing cells. In some embodiments, the biomaterials is comprised in a composition that further comprises one or more of a cell recruitment composition, a viral vector, and/or a cell activation agent.

### BACKGROUND OF THE INVENTION

T cell adoptive transfer protocols show potential in a number of therapeutic applications, such as cancer, where CAR T cell therapies have recently been approved for the treatment of B cell malignancies. Current methodologies of CAR-T cell manufacture are performed ex vivo: extracting cells from a subject, engineering them to express a chimeric antigen receptor (CAR), and then reintroducing them into a subject for treatment of a disease, disorder, or condition, such as cancer. There remains a need in the art for efficient manufacturing of CAR expressing cells, including but not limited to, those which allow for site-specific delivery and/or in vivo production.

### SUMMARY OF THE INVENTION

In some aspects, the disclosure features is a first composition comprising a biomaterial and a cell recruitment factor; and a second composition comprising a viral vector. In some aspects, the disclosure features is a first composition comprising a biomaterial and a molecule (e.g., VEGF-C, IL-2, IL-7, IL-15 (e.g., hetIL-15 (IL15/sIL-15Ra)), GM-CSF, CXCL12, CXC3L1, CCL19, CCL21, CXCL10, or CXCL11); and a second composition comprising a viral vector.

In some aspects, the disclosure features a first composition comprising a biomaterial and a cell recruitment factor, wherein the biomaterial comprises a hydrogel, e.g., a cryogel, e.g., an alginate cryogel, and wherein the cell recruitment factor comprises an amino acid sequence according to SEQ ID NO: 741, or an amino acid sequence with at least 80%, 85%, 90%, 95%, or 99% sequence identity thereto, provided that the amino acid at position 26 of SEQ ID NO: 741 is not Cysteine (C), optionally wherein the amino acid at position 26 of SEQ ID NO: 741 is Alanine (A).

In some aspects, the disclosure features a second composition comprising a mesoporous silica particle; a viral vector; and a cell activation agent.

In some aspects, the disclosure features a method of transducing cells of a subject in vivo or treating a disease, disorder, or condition in a subject. The method comprises: administering a biomaterial and a cell recruitment factor to a site (e.g., high subcutaneous space or subcutaneous space adjacent to dermis) in the subject, and administering a viral vector or a nucleic acid comprising a transgene to the subject; thereby transducing cells of the subject with the transgene. In some aspects, the disclosure features a method of transducing cells of a subject in vivo or treating a disease, disorder, or condition in a subject. The method comprises: administering a biomaterial and a molecule (e.g., VEGF-C, IL-2, IL-7, IL-15 (e.g., hetIL-15 (IL15/sIL-15Ra)), GM-CSF, CXCL12, CXC3L1, CCL19, CCL21, CXCL10, or CXCL11) to a site (e.g., high subcutaneous space or subcutaneous space adjacent to dermis) in the subject, and administering a viral vector or a nucleic acid comprising a transgene to the subject; thereby transducing cells of the subject with the transgene.

In some embodiments, the biomaterial and the cell recruitment factor are comprised in a first composition, and the viral vector or nucleic acid is comprised in a second composition. In some embodiments, the biomaterial and the molecule (e.g., VEGF-C, IL-2, IL-7, IL-15 (e.g., hetIL-15 (IL15/sIL-15Ra)), GM-CSF, CXCL12, CXC3L1, CCL19, CCL21, CXCL10, or CXCL11) are comprised in a first composition, and the viral vector or nucleic acid is comprised in a second composition.

In some aspects, the disclosure features a method of transducing cells of a subject in vivo or treating a disease, disorder, or condition in a subject, the method comprising: administering a viral vector or a nucleic acid comprising a transgene to a site in the subject, wherein the subject has previously been administered a biomaterial and a cell recruitment factor in an amount sufficient to induce lymphangiogenesis and/or recruitment of T cells to the site in the subject; thereby transducing the cells. In some aspects, the disclosure features a method of transducing cells of a subject in vivo or treating a disease, disorder, or condition in a subject, the method comprising: administering a viral vector or a nucleic acid comprising a transgene to a site in the subject, wherein the subject has previously been administered a biomaterial and a molecule (e.g., VEGF-C, IL-2, IL-7, IL-15 (e.g., hetIL-15 (IL15/sIL-15Ra)), GM-CSF, CXCL12, CXC3L1, CCL19, CCL21, CXCL10, or CXCL11) in an amount sufficient to induce lymphangiogenesis and/or recruitment of T cells to the site in the subject; thereby transducing the cells.

In any of the aspects herein, e.g., the compositions and methods above, any of the embodiments herein (e.g., below) may apply.

In some embodiments, the biomaterial comprises (i) comprises a hydrogel; (ii) comprises a cryogel; (iii) comprises a gelatin, hyaluronic acid, collagen, alginate, laminin, chitosan, silk fibroin, agarose, poly(ethylene glycol), poly-vinyl alcohol, and/or hydroxyethyl methylacrylate; (iv) comprises alginate hydrogel, optionally wherein the alginate hydrogel further comprises norbornene and/or tetrazine, optionally wherein the norbornene and/or tetrazine is covalently associated with, e.g., chemically linked to, or non-covalently associated with, e.g., adsorbed on, the alginate; and/or (v) comprises pores between about 10 μm to about 300 μm, e.g., between about 50 μm to about 300 μm, in diameter, or no pores; and/or (vi) is chemically crosslinked. In some embodiments, the first composition comprising the biomaterial further comprises laponite, optionally wherein the laponite is present at a concentration of about 0.15 mg/mL to about 0.35 mg/mL, e.g., about 0.25 mg/mL. In some embodiments, the biomaterial further comprises laponite, optionally wherein the laponite is present at a concentration of about 0.15 mg/mL to about 0.35 mg/mL, e.g., about 0.25 mg/mL.

In some embodiments, the cell recruitment factor is: (i) noncovalently associated with, e.g., adsorbed on, the biomaterial; or (ii) covalently associated with, e.g., conjugated to, the biomaterial. In some embodiments, the cell recruitment factor: (i) induces lymphangiogenesis; (ii) induce growth of lymphatic endothelial cells; and/or (ii) recruits immune cells, optionally wherein the immune cells comprise T-cells and/or NK-cells.

In some embodiments, induction of lymphangiogenesis: (i) comprises an increase in the level of lymphatic endothelial cells (LECs) (e.g., CD45−CD31+PDPN+ cells), optionally wherein the level of LECs is increased by at least 10%, 20%, 30%, 40%, 50%, 60, 70%, 75%, 80%, 85%, 90%, 95%, 100%, or 2 00% as compared to a reference level (e.g., the level of LECs at a site in a subject prior to injection of the plurality of compositions or the first composition), when measured by an assay, e.g., a flow cytometry assay, e.g., as described in Example H or I; and/or (ii) results in at least 50 LECs (e.g., at least 75, 100, 125, 150, 200, 225, or 250 LECs) per milligram of tissue when measured by an assay, e.g., a flow cytometry assay, e.g., as described in Example H or I.

In some embodiments, the cell recruitment factor recruits T cells, optionally wherein the T cells comprise naive T cells (e.g., CD45RA+CD62L+ T cells or CD45RA+CD62L+CCR7+CD27+CD95+ T cells). In some embodiments, recruitment of T cells comprises an increase in the level of T cells, optionally wherein the level of T cells is increased by at least 10%, 20%, 30%, 40%, 50%, 60, 70%, 75%, 80%, 85%, 90%, 95% 100%, 200%, or 300% as compared to a reference level (e.g., the level of T cells at a site in a subject prior to injection of the plurality of compositions or the first composition), when measured by an assay, e.g., a flow cytometry assay, e.g., as described in Example H or I.

In some embodiments, the cell recruitment factor is chosen from VEGF-C, IL-2, IL-7, IL-15 (e.g., hetIL-15 (IL15/sIL-15Ra)), GM-CSF, CXCL12, CXC3L1, CCL19, CCL21, CXCL10, or CXCL11. In some embodiments, the cell recruitment factor comprises VEGF-C or a functional variant thereof; IL-15 (e.g., hetIL-15 (IL15/sIL-15Ra)) or a functional variant thereof, IL-7 or a functional variant thereof; or a combination thereof.

In some embodiments, the cell recruitment factor comprises VEGF-C, optionally wherein the VEGF-C: (i) comprises a mature VEGF-C peptide, optionally the minor or major mature form or a mutated variant thereof; (ii) is a monomer or dimer; and/or (iii) is present in an effective amount, optionally, in an amount of less than or about 1 mg, less than or about 10 mg, greater than or about 10 μg, greater than or about 1 μg, between about 1 μg and 1 mg, between about 10 μg and 1 mg, between about 1 μg and 10 mg, or between about 10 μg and 10 mg.

In some embodiments, the VEGF-C comprises: (i) an amino acid sequence of any one of the sequences provided in Table 18 or a sequence with at least 95% sequence identity thereto, optionally wherein the sequence comprises or does not comprise a linker (e.g., a glycine-serine linker) and/or a his tag; and/or (ii) the amino acid substitution of C137A, numbered according to SEQ ID NO: 725.

In some embodiments, the cell recruitment factor comprises: (i) an amino acid sequence according to SEQ ID NO: 741, or an amino acid sequence with at least 80%, 85%, 90%, 95%, or 99% sequence identity thereto, provided that the amino acid at position 26 of SEQ ID NO: 741 is not Cysteine (C), optionally wherein the amino acid at position 26 of SEQ ID NO: 741 is Alanine (A); (ii) the amino acid sequence according to SEQ ID NO: 743 or a sequence an amino acid sequence with at least 80%, 85%, 90%, 95%, or 99% sequence identity thereto; (iii) the amino acid sequence according to SEQ ID NO: 740 or an amino acid sequence with at least 80%, 85%, 90%, 95%, or 99% sequence identity thereto; (iv) the amino acid sequence according to SEQ ID NO: 736, or an amino acid sequence with at least 80%, 85%, 90%, 95%, or 99% sequence identity thereto; (v) a linker, e.g., wherein the linker has a sequence of Gly-Ser, wherein optionally the linker is C-terminal of SEQ ID NO: 743 or a sequence having at least 80%, 85%, 90%, 95%, or 99% sequence identity thereto; (vi) the amino acid sequence according to SEQ ID NO: 735, or an amino acid sequence with at least 80%, 85%, 90%, 95%, or 99% sequence identity thereto; (vii) the amino acid sequence according to SEQ ID NO: 734, or an amino acid sequence with at least 80%, 85%, 90%, 95%, or 99% sequence identity thereto; and/or (viii) the amino acid sequence according to SEQ ID NO: 733, or an amino acid sequence with at least 80%, 85%, 90%, 95%, or 99% sequence identity thereto.

In some embodiments, any of the compositions, e.g., any of the first compositions, described herein further comprises IL-15 (e.g., hetIL-15 (IL15/sIL-15Ra)) or a functional variant thereof In some embodiments, any of the compositions, e.g., any of the first compositions, described herein further comprises IL-7 or a functional variant thereof In some embodiments, any of the compositions, e.g., any of the first compositions, described herein further comprises IL-15 (e.g., hetIL-15 (IL15/sIL-15Ra)) or a functional variant thereof and IL-7 or a functional variant thereof.

In some embodiments, any of the methods described herein further comprises administration of IL-15 (e.g., hetIL-15 (IL15/sIL-15Ra)) or a functional variant thereof. In some embodiments, any of the methods described herein further comprises administration of IL-7 or a functional variant thereof. In some embodiments, any of the methods described herein further comprises administration IL-15 (e.g., hetIL-15 (IL15/sIL-15Ra)) and a functional variant thereof or IL-7 or a functional variant thereof.

In some embodiments, the second composition further comprises a particle. In some embodiments, the particle is a mesoporous particle, a silica particle and/or a mesoporous silica particle, optionally wherein the mesoporous silica particle is a mesoporous silica rod. In some embodiments, the mesoporous silica particle comprises a surface modification, optionally wherein the surface modification comprises: (a) a —OH (hydroxyl), amine, carboxylic acid, phosphonate, halide, azide, alkyne, epoxide, sulfhydryl, polyethyleneimine, a hydrophobic moiety, or salts thereof, optionally using a C1 to C20 alkyl or (O(CH2 CH2)1-25 linker; (b) a primary, secondary, tertiary, or quaternary amine; and/or (c) a polyethyleneimine having an average molecular weight of about 1000 to 20,000 Da, about 1,200 to 15,000 Da, about 1,500 to 12,000 Da, about 2,000 Da, about 3,000 Da, about 4,000 Da, about 5,000 Da, about 6,000 Da, about 7,000 Da, about 8,000 Da, about 9,000 Da, or about 10,000 Da, as measured by gel permeation chromatography (GPC). In some embodiments, the mesoporous silica particle (i) is a trimethylammonium functionalized mesoporous silica particle, e.g., a N,N,N-trimethylpropan-1-ammonium functionalized mesoporous silica particle; (iii) comprises a plurality of pores, optionally wherein the pores are between 2-50 nm in diameter; and/or (iv) comprises a surface area of at least about 100 m2/g.

In some embodiments, (i) the viral vector is noncovalently, e.g., electrostatically, or covalently associated with the mesoporous silica particle; and/or (ii) the cell activation agent is noncovalently or covalently associated with the mesoporous silica particle. In some embodiments, the viral vector comprises: (i) a lentivirus, retrovirus, adenovirus, adeno-associated virus, or herpes virus; and/or (ii) an expression vector comprising a recombinant polynucleotide comprising an expression control sequence operatively linked to a nucleotide sequence to be expressed. In some embodiments, the nucleotide sequence encodes: a chimeric antigen receptor (CAR), an engineered TCR, a cytokine, a chemokine, an shRNA, or a polypeptide engineered to target a tumor antigen.

In some embodiments, the tumor antigen is selected from the group consisting of: TSHR, CD19, CD123, CD22, CD30, CD171, CS-1, CLL-1, CD33, EGFRvIII, GD2, GD3, BCMA, Tn Ag, PSMA, ROR1, FLT3, FAP, TAG72, CD38, CD44v6, CEA, EPCAM, B7H3, KIT, IL-13Ra2, Mesothelin, IL-11Ra, PSCA, PRSS21, VEGFR2, LewisY, CD24, PDGFR-beta, SSEA-4, CD20, Folate receptor alpha, ERBB2 (Her2/neu), MUC1, EGFR, NCAM, Prostase, PAP, ELF2M, Ephrin B2, IGF-I receptor, CAIX, LMP2, gp100, bcr-abl, tyrosinase, EphA2, Fucosyl GM1, sLe, GM3, TGS5, HMWMAA, o-acetyl-GD2, Folate receptor beta, TEM1/CD248, TEM7R, CLDN6, GPRC5D, CXORF61, CD97, CD179a, ALK, Polysialic acid, PLAC1, GloboH, NY-BR-1, UPK2, HAVCR1, ADRB3, PANX3, GPR20, LY6K, OR51E2, TARP, WT1, NY-ESO-1, LAGE-1a, MAGE-A1, legumain, HPV E6, E7, MAGE A1, ETV6-AML, sperm protein 17, XAGE1, Tie 2, MAD-CT-1, MAD-CT-2, Fos-related antigen 1, p53, p53 mutant, prostein, survivin and telomerase, PCTA-1/Galectin 8, MelanA/MART1, Ras mutant, hTERT, sarcoma translocation breakpoints, ML-IAP, ERG (TMPRSS2 ETS fusion gene), NA17, PAX3, Androgen receptor, Cyclin B1, MYCN, RhoC, TRP-2, CYP1B1, BORIS, SART3, PAX5, OY-TES1, LCK, AKAP-4, SSX2, RAGE-1, human telomerase reverse transcriptase, RU1, RU2, intestinal carboxyl esterase, mut hsp70-2, CD79a, CD79b, CD72, LAIR1, FCAR, LILRA2, CD300LF, CLEC12A, BST2, EMR2, LY75, GPC3, FCRL5, IGLL1, and any combination thereof.

In some embodiments, the viral vector encodes a CAR that comprises an antigen binding domain, a transmembrane domain, a costimulatory signaling region, and a signaling domain, wherein: (i) the antigen binding domain binds an antigen selected from the group consisting of CD19, CD123, CD22, CD20, EGFRvIII, BCMA, Mesothelin, CD33, CLL-1, and any combination thereof; (ii) the transmembrane domain comprises a CD8 hinge; (iii) the costimulatory signaling region is selected from a 4-1BB or CD28 costimulatory signaling domain; and/or (iv) the signaling domain comprises a CD3 zeta signaling domain.

In some embodiments, the second composition further comprises a cell activation agent. In some embodiments, the cell activation agent comprises an agent that stimulates CD3/TCR complex and/or an agent that stimulates a costimulatory molecule and/or growth factor receptor. In some embodiments, the cell activation agent comprises a multispecific binding molecule comprising: (A) an anti-CD3 binding domain, and (B) a costimulatory molecule binding domain (e.g., an anti-CD2 binding domain or an anti-CD28 binding domain).

In some embodiments, the anti-CD3 binding domain, e.g., an anti-CD3 scFv, is situated N-terminal of the costimulatory molecule binding domain, e.g., an anti-CD2 Fab or an anti-CD28 Fab; or the anti-CD3 binding domain, e.g., an anti-CD3 scFv, is situated C-terminal of the costimulatory molecule binding domain, e.g., an anti-CD2 Fab or an anti-CD28 Fab, optionally wherein: an Fc region is situated between the anti-CD3 binding domain and the costimulatory molecule binding domain; or the multispecific binding molecule comprises a CH2, and the anti-CD3 binding domain is situated N-terminal of the CH2.

In some embodiments of the second compositions and methods herein, the multispecific binding molecule comprises: (i) a first polypeptide comprising from N-terminal to C-terminal: VH of the anti-CD3 binding domain, VL of the anti-CD3 binding domain, VH of the costimulatory molecule binding domain, CH1, CH2, and CH3; and (ii) a second polypeptide comprising from N-terminal to C-terminal: VL of the costimulatory molecule binding domain and CL. In some embodiments, the multispecific binding molecule comprises: (i) a first polypeptide comprising from N-terminal to C-terminal: VH of the costimulatory molecule binding domain, CH1, CH2, CH3, VH of the anti-CD3 binding domain, and VL of the anti-CD3 binding domain; and (ii) a second polypeptide comprising from N-terminal to C-terminal: VL of the costimulatory molecule binding domain and CL. In some embodiments, the multispecific binding molecule comprises: (i) a first polypeptide comprising from N-terminal to C-terminal: VH of the costimulatory molecule binding domain, CH1, VH of the anti-CD3 binding domain, VL of the anti-CD3 binding domain, CH2, and CH3; and (ii) a second polypeptide comprising from N-terminal to C-terminal: VL of the costimulatory molecule binding domain and CL. In some embodiments, the anti-CD3 binding domain comprises an scFv and the costimulatory molecule binding domain is part of a Fab fragment.

In some embodiments, the cell activation agent comprises the amino acid sequence of any heavy chain provided in Table 20, or an amino acid sequence with at least 95% sequence identity thereto; and/or the amino acid sequence of any light chain provided in Table 20, or an amino acid sequence with at least 95% sequence identity thereto. In some embodiments, the cell activation agent is conjugated to or adsorbed on the particle, e.g., mesoporous silica particle.

In some embodiments, the multispecific binding molecule comprises an Fc region comprising: (i) a L234A, L235A, S267K, and P329A mutation (LALASKPA), numbered according to the Eu numbering system; (ii) a L234A, L235A, and P329G mutation (LALAPG), numbered according to the Eu numbering system; (iii) a G237A, D265A, P329A, and S267K mutation (GADAPASK), numbered according to the Eu numbering system; (iv) a L234A, L235A, and G237A mutation (LALAGA), numbered according to the Eu numbering system; (v) a D265A, P329A, and S267K mutation (DAPASK), numbered according to the Eu numbering system; (vi) a G237A, D265A, and P329A mutation (GADAPA), numbered according to the Eu numbering system; (vii) a L234A, L235A, and P329A mutation (LALAPA), numbered according to the Eu numbering system; or (viii) an amino acid sequence of any of the Fc regions in Table 20 or an amino acid sequence having at least 95% identity thereto.

In some embodiments, the multispecific binding molecule comprises: (i) a heavy chain comprising the amino acid sequence of any of SEQ ID NOs: 726, 1416, 893, 1417, or 895, or an amino acid sequence having at least 95% sequence identity thereto; and/or (ii) a light chain comprising the amino acid sequence of any of SEQ ID NOs: 728, 730, 892, or 894, or an amino acid sequence having at least 95% sequence identity thereto. In some embodiments, the multispecific binding molecule comprises: (i) a heavy chain comprising the amino acid sequence of SEQ ID NO: 726 or an amino acid sequence having at least 95% sequence identity thereto, and a light chain comprising the amino acid sequence of SEQ ID NO: 728, or an amino acid sequence having at least 95% sequence identity thereto; (ii) a heavy chain comprising the amino acid sequence of SEQ ID NO: 726 or an amino acid sequence having at least 95% sequence identity thereto, and a light chain comprising the amino acid sequence of SEQ ID NO: 730, or an amino acid sequence having at least 95% sequence identity thereto; (iii) a heavy chain comprising the amino acid sequence of SEQ ID NO: 1416 or an amino acid sequence having at least 95% sequence identity thereto, and a light chain comprising the amino acid sequence of SEQ ID NO: 728, or an amino acid sequence having at least 95% sequence identity thereto; (iv) a heavy chain comprising the amino acid sequence of SEQ ID NO: 1416 or an amino acid sequence having at least 95% sequence identity thereto, and a light chain comprising the amino acid sequence of SEQ ID NO: 730, or an amino acid sequence having at least 95% sequence identity thereto; (v) a heavy chain comprising the amino acid sequence of SEQ ID NO: 893 or an amino acid sequence having at least 95% sequence identity thereto, and a light chain comprising the amino acid sequence of SEQ ID NO: 892, or an amino acid sequence having at least 95% sequence identity thereto; (vi) a heavy chain comprising the amino acid sequence of SEQ ID NO: 1417 or an amino acid sequence having at least 95% sequence identity thereto, and a light chain comprising the amino acid sequence of SEQ ID NO: 892, or an amino acid sequence having at least 95% sequence identity thereto; or (vii) a heavy chain comprising the amino acid sequence of SEQ ID NO: 895 or an amino acid sequence having at least 95% sequence identity thereto, and a light chain comprising the amino acid sequence of SEQ ID NO: 894, or an amino acid sequence having at least 95% sequence identity thereto.

In some embodiments, the second composition further comprises a first population of particles and a second population of particles, e.g., a first population of mesoporous silica particles and a second population of mesoporous silica particles, wherein the first population comprises the viral vector and the second population comprises a cell activation agent, e.g., wherein the viral vector is noncovalently associated with a particle of the first population and the cell activation agent is noncovalently associated with a particle of the second population.

In some embodiments, the composition, e.g., the first or second composition, is suitable for injectable use.

In some embodiments, the composition, e.g., the first or second composition, described herein further comprises a Tet2 inhibitor and/or a ZBTB32 inhibitor. In some embodiments, the Tet2 inhibitor comprises: (1) a gene editing system targeted to one or more sites within the gene encoding Tet2, or its corresponding regulatory elements; (2) a nucleic acid (e.g., an siRNA or shRNA) that inhibits expression of Tet2; (3) a protein (e.g., a dominant negative, e.g., catalytically inactive) Tet2, or a binding partner of Tet2 (e.g., a dominant negative binding partner of Tet2); (4) a small molecule that inhibits expression and/or function of Tet2; (5) a nucleic acid encoding any of (1)-(3); or (6) any combination of (1)-(5). In some embodiments, the ZBTB32 gene or one or more components thereof, (2) a nucleic acid encoding one or more components of the gene editing system; or (3) a combination of (1) and (2). In an embodiment, the ZBTB32 inhibitor comprises: (1) a gene editing system targeting the ZBTB32 gene or one or more components thereof. In an embodiment, the ZBTB32 inhibitor comprises (2) a nucleic acid encoding one or more components of the gene editing system. In an embodiment, the ZBTB32 inhibitor comprises a combination of (1) and (2).

In some embodiments, the methods described herein further comprise administering to the subject: (i) a Tet2 inhibitor, optionally wherein the Tet2 inhibitor comprises: (1) a gene editing system targeted to one or more sites within the gene encoding Tet2, or its corresponding regulatory elements; (2) a nucleic acid (e.g., an siRNA or shRNA) that inhibits expression of Tet2; (3) a protein (e.g., a dominant negative, e.g., catalytically inactive) Tet2, or a binding partner of Tet2 (e.g., a dominant negative binding partner of Tet2); (4) a small molecule that inhibits expression and/or function of Tet2; (5) a nucleic acid encoding any of (1)-(3); or (6) any combination of (1)-(5); and/or (ii) a ZBTB32 inhibitor, optionally wherein the ZBTB32 inhibitor comprises: (1) a gene editing system targeting the ZBTB32 gene or one or more components thereof, (2) a nucleic acid encoding one or more components of the gene editing system; or (3) a combination of (1) and (2). In an embodiment, the ZBTB32 inhibitor comprises: (1) a gene editing system targeting the ZBTB32 gene or one or more components thereof. In an embodiment, the ZBTB32 inhibitor comprises (2) a nucleic acid encoding one or more components of the gene editing system. In an embodiment, the ZBTB32 inhibitor comprises a combination of (1) and (2) In some embodiments, the first composition is administered prior to the administration of the second composition, optionally wherein: (i) the first composition is administered about 1-4 weeks, e.g., about 2 weeks, prior to the administration of the second composition; or (ii) the first composition is administered at least two weeks prior to the administration of the second composition.

In some embodiments, any of the methods described herein further comprises evaluating, e.g., measuring, lymphangiogenesis in a sample from the subject (e.g., a sample from or close to the site of administration), wherein lymphangiogenesis is measured after the administration of the first composition and/or prior to the administration of the second composition, optionally wherein measuring lymphangiogenesis comprises acquiring a value for the level and/or activity of lymphatic endothelial cells (LECs) (e.g., CD45−CD31+PDPN+ cells) in the sample.

In some embodiments, any of the methods described herein further comprises evaluating, e.g., measuring, the recruitment of T cells in a sample from the subject (e.g., a sample from or close to the site of administration), wherein the recruitment of T cells is measured after the administration of the first composition and/or prior to the administration of the second composition, optionally wherein measuring the recruitment of T cells comprises acquiring a value for the level and/or activity of T cells (e.g., naïve T cells, e.g., CD45RA+CD62L+ T cells and/or CD45RA+CD62L+CCR7+CD27+CD95+ T cells) in the sample.

In some embodiments, the subject has or has been diagnosed with having a disease, disorder, or condition; and/or the subject is a human.

In some embodiments, the disease, disorder, or condition comprises: (i) a cancer; (ii) a hematological cancer, optionally wherein the hematological cancer comprises a leukemia or lymphoma; (iii) a hematological cancer chosen from chronic lymphocytic leukemia (CLL), mantle cell lymphoma (MCL), multiple myeloma, acute lymphoid leukemia (ALL), Hodgkin lymphoma, B-cell acute lymphoid leukemia (BALL), T-cell acute lymphoid leukemia (TALL), small lymphocytic leukemia (SLL), B cell prolymphocytic leukemia, blastic plasmacytoid dendritic cell neoplasm, Burkitt [symbol] lymphoma, diffuse large B cell lymphoma (DLBCL), DLBCL associated with chronic inflammation, chronic myeloid leukemia, myeloproliferative neoplasms, follicular lymphoma, pediatric follicular lymphoma, hairy cell leukemia, small cell- or a large cell-follicular lymphoma, malignant lymphoproliferative conditions, MALT lymphoma (extranodal marginal zone lymphoma of mucosa-associated lymphoid tissue), Marginal zone lymphoma, myelodysplasia, myelodysplastic syndrome, non-Hodgkin lymphoma, plasmablastic lymphoma, plasmacytoid dendritic cell neoplasm, Waldenstrom macroglobulinemia, splenic marginal zone lymphoma, splenic lymphoma/leukemia, splenic diffuse red pulp small B-cell lymphoma, hairy cell leukemia-variant, lymphoplasmacytic lymphoma, a heavy chain disease, plasma cell myeloma, solitary plasmocytoma of bone, extraosseous plasmocytoma, nodal marginal zone lymphoma, pediatric nodal marginal zone lymphoma, primary cutaneous follicle center lymphoma, lymphomatoid granulomatosis, primary mediastinal (thymic) large B-cell lymphoma, intravascular large B-cell lymphoma, ALK+ large B-cell lymphoma, large B-cell lymphoma arising in HHV8-associated multicentric Castleman disease, primary effusion lymphoma, B-cell lymphoma, acute myeloid leukemia (AML), or unclassifiable lymphoma; (iv) a solid cancer; (v) a solid cancer chosen from: one or more of mesothelioma, malignant pleural mesothelioma, non-small cell lung cancer, small cell lung cancer, squamous cell lung cancer, large cell lung cancer, pancreatic cancer, pancreatic ductal adenocarcinoma, esophageal adenocarcinoma, breast cancer, glioblastoma, ovarian cancer, colorectal cancer, prostate cancer, cervical cancer, skin cancer, melanoma, renal cancer, liver cancer, brain cancer, thymoma, sarcoma, carcinoma, uterine cancer, kidney cancer, gastrointestinal cancer, urothelial cancer, pharynx cancer, head and neck cancer, rectal cancer, esophagus cancer, or bladder cancer, or a metastasis thereof, or (iv) an autoimmune disease, an inflammatory disease, or a transplant. In some embodiments, the disease, disorder, or condition is an autoimmune disease, an inflammatory disease, or a transplant and the CAR expressed binds to a B cell antigen, e.g., CD 19, CD20, CD22, CD123, FcRn5, FcRn2, BCMA, CS-1 and CD138.

In some embodiments, the disease, disorder, or condition comprises a solid tumor. In some embodiments, when treating a solid tumor with a CAR-expressing cell manufactured using the methods described herein, the cell expresses two CARs, the first CAR binds to a B cell antigen and the second CAR binds to a solid tumor antigen. In some embodiments, when treating a solid tumor with a CAR-expressing cell manufactured using the methods described herein, the cell expresses a multispecific CAR that comprises a first binding domain that binds to a B cell antigen and a second binding domain that binds to a solid tumor antigen. Without wishing to be bound by theory, expressing a CAR that binds to a B cell antigen in such cells may help improve the proliferation and/or survival of the CAR-expressing cells. B cell antigens (e.g., CD19 on normal B cells) may improve the proliferation and/or survival of such CAR-expressing cells even when the level of the tumor antigen is low (e.g., when the number of tumor cells is low, or before the CAR-expressing cells manufactured using the methods described herein encounter solid tumor cells).

In some embodiments, the disease, disorder, or condition comprises a myeloid tumor. In some embodiments, when treating a myeloid tumor with a CAR-expressing cell manufactured using the methods described herein, the cell expresses two CARs, the first CAR binds to a B cell antigen and the second CAR binds to a myeloid tumor antigen. In some embodiments, when treating a myeloid tumor with a CAR-expressing cell manufactured using the methods described herein, the cell expresses a multispecific CAR that comprises a first binding domain that binds to a B cell antigen and a second binding domain that binds to a myeloid tumor antigen. Without wishing to be bound by theory, expressing a CAR that binds to a B cell antigen in such cells may help improve the proliferation and/or survival of the CAR-expressing cells. B cell antigens (e.g., CD19 on normal B cells) may improve the proliferation and/or survival of such CAR-expressing cells even when the level of the tumor antigen is low (e.g., when the number of tumor cells is low, or before the CAR-expressing cells manufactured using the methods described herein encounter myeloid tumor cells).

In some embodiments of the methods or second compositions described herein, the viral vector or the nucleic acid encodes:
(i) a first CAR that binds to a B cell antigen (e.g., CD19) and a second CAR that binds to (a) a solid tumor antigen (e.g., EGFRvIII), (b) a myeloid tumor antigen, or (c) an antigen of a hematological tumor not of B-cell lineage; or
(2) a CAR that comprises a first binding domain that binds to a B cell antigen (e.g., CD19) and a second binding domain that binds to (a) a solid tumor antigen (e.g., EGFRvIII), (b) a myeloid tumor antigen, or (c) an antigen of a hematological tumor not of B-cell lineage.

In some embodiments, the B cell antigen is CD5, CD10, CD19, CD20, CD21, CD22, CD23, CD24, CD25, CD27, CD30, CD34, CD37, CD38, CD40, CD53, CD69, CD72, CD73, CD74, CD75, CD77, CD79a, CD79b, CD80, CD81, CD82, CD83, CD84, CD85, CD86, CD123, CD135, CD138, CD179, CD269, Flt3, ROR1, BCMA, FcRn5, FcRn2, CS-1, CXCR4, 5, 7, IL-7/3R, 1L7/4/3R, or IL4R. In some embodiments, the B cell antigen is CD19, CD20, CD22, CD123, FcRn5, FcRn2, BCMA, CS-1 or CD138.

In some embodiments, the solid tumor antigen is EGFRvIII, mesothelin, GD2, Tn Ag, PSMA, TAG72, CD44v6, CEA, EPCAM, KIT, IL-13Ra2, leguman, GD3, CD171, IL-11Ra, PSCA, MAD-CT-1, MAD-CT-2, VEGFR2, LewisY, CD24, PDGFR-beta, SSEA-4, folate receptor alpha, ERBBs (e.g., ERBB2), Her2/neu, MUC1, EGFR, NCAM, Ephrin B2, CAIX, LMP2, sLe, HMWMAA, o-acetyl-GD2, folate receptor beta, TEM1/CD248, TEM7R, FAP, Legumain, HPV E6 or E7, ML-IAP, CLDN6, TSHR, GPRC5D, ALK, Polysialic acid, Fos-related antigen, neutrophil elastase, TRP-2, CYP1B1, sperm protein 17, beta human chorionic gonadotropin, AFP, thyroglobulin, PLAC1, globoH, RAGE1, MN-CA IX, human telomerase reverse transcriptase, intestinal carboxyl esterase, mut hsp 70-2, NA-17, NY-BR-1, UPK2, HAVCR1, ADRB3, PANX3, GPR20, Ly6k, OR51E2, TARP, GFRa4, or a peptide of any of these antigens presented on MHC.

In some embodiments, also contemplated herein is a kit comprising a first composition described herein and a second composition described herein.

In some aspects, the disclosure features a method of treating a disease, disorder, or condition in a subject, the method comprising: administering a viral vector or a nucleic acid comprising a transgene to a site in the subject that has been induced to undergo lymphangiogenesis at the site, thereby transducing the cells.

In some aspects, the disclosure features a method of preparing a subject to receive a viral vector encoding a chimeric antigen receptor (CAR), comprising administering to the subject a biomaterial and a cell recruitment factor, thereby preparing the subject to receive the viral vector encoding the CAR. In some aspects, the disclosure features a method of preparing a subject to receive a viral vector encoding a chimeric antigen receptor (CAR), comprising administering to the subject a biomaterial and a molecule (e.g., VEGF-C, IL-2, IL-7, IL-15 (e.g., hetIL-15 (IL15/sIL-15Ra)), GM-CSF, CXCL12, CXC3L1, CCL19, CCL21, CXCL10, or CXCL11), thereby preparing the subject to receive the viral vector encoding the CAR.

In some embodiments, preparing comprises induction of lymphangiogenesis and/or recruitment of T cells (e.g., e.g., CD45RA+CD62L+ T cells and/or CD45RA+CD62L+CCR7+CD27+CD95+ T cells). In some embodiments, the method further comprises administering the viral vector encoding the CAR, optionally wherein the viral vector is conjugated to a particle (e.g., a mesoporous silica particle that comprises or does not comprise a cell activation agent).

Also contemplated herein is a composition, comprising a biomaterial comprising a cell recruitment factor; a first population of mesoporous silica particles; a viral vector; and, optionally, a cell activation agent. Also contemplated herein is a composition, comprising a biomaterial and a cell recruitment factor; a first population of mesoporous silica particles; a viral vector; and, optionally, a cell activation agent.

In some embodiments, the biomaterial comprises a hydrogel, optionally a cryogel. In some embodiments, the cryogel comprises gelatin, hyaluronic acid, collagen, alginate, laminin, chitosan, silk fibroin, agarose, poly(ethylene glycol), poly-vinyl alcohol, and/or hydroxyethyl methylacrylate. In some embodiments, the composition comprising the cryogel further comprises laponite. In some embodiments, the cryogel comprises pores between about 10 to 300 μm in diameter, optionally between about 50 to 300 μm. In some embodiments, the cryogel is chemically cross-linked.

In some embodiments, the cell recruitment factor selectively recruits immune cells, optionally T-cells and/or NK-cells. In some embodiments, the cell recruitment factor is selected from the group consisting of CCL19, CXCL9, CXCL10, XCL1, IL-2, IL-7, CCL21, GM-CSF, CCL17, CCL22, CCL20, CCL27, IL-15, lymphotoxin alpha, lymphotoxin beta, VEGF-C, FLT3L, G-CSF, PDGF, S100A8/A9, CSF-1, CXCL8, CCL20, CCL17, CCR5, CCR6, CCL2, VEGF, Angiopoietin-2, PGE2, LTB4, CXC3L1, CCL19, CCL21, CXCL10, CXCL11, and/or CXCL12. In some embodiments, the VEGF-C is selected from the group consisting of immature VEGF-C peptide or mature VEGF-C peptide. In some embodiments, the mature VEGF-C peptide is the minor mature form or major mature form. In some embodiments, the mature VEGF-C peptide is a wild type minor mature form or a wild type major mature form. In some embodiments, the mature VEGF-C peptide is a modified minor mature form or a modified major mature form. In some embodiments, the mature VEGF-C peptide is a modified minor mature form comprising a mutation at Cysteine 137 (e.g., C137A) or a modified major mature form comprising a mutation at Cysteine 137 (e.g., C137A). In some embodiments, the mature VEGF-C peptide is a modified minor mature form comprising a C137A mutation or a modified major mature form comprising a C137A mutation. In some embodiments, the mature VEGF-C peptide is present as a dimer or monomer. In some embodiments, the VEGF-C is a dimer of the major mature form further comprising a C137A mutation in each monomer. In some embodiments, the VEGF-C is a dimer of the minor mature form further comprising a C137A mutation in each monomer. In some embodiments, the VEGF-C is selected from a sequence provided in Table 18, optionally, wherein the his tag is not included in the sequence. In some embodiments, the VEGF-C is present in an effective amount, optionally in an amount of less than or about 1 mg, less than or about 10 mg, greater than or about 10 μg, greater than or about 1 μg, between about 1 μg and 1 mg, between about 10 μg and 1 mg, between about 1 μg and 10 mg, or between about 10 μg and 10 mg.

In some embodiments, the first population of mesoporous silica particles are surface modified. In some embodiments, the surface modification on the first population of mesoporous silica particles is —OH (hydroxyl), amine, carboxylic acid, phosphonate, halide, azide, alkyne, epoxide, sulfhydryl, polyethyleneimine, a hydrophobic moiety, or salts thereof, optionally using a C₁ to C₂₀ alkyl or (—O(CH2-CH₂—)₁₋₂₅ linker. In some embodiments, the surface modification on the first population of mesoporous silica particles is a primary, secondary, tertiary, or quaternary amine. In some embodiments, the surface modification on the first population of mesoporous silica particles is a polyethyleneimine having an average molecular weight of about 1000 to 20,000 Da, about 1,200 to 15,000 Da, about 1,500 to 12,000 Da, about 2,000 Da, about 3,000 Da, about 4,000 Da, about 5,000 Da, about 6,000 Da, about 7,000 Da, about 8,000 Da, about 9,000 Da, or about 10,000 Da, as measured by gel permeation chromatography (GPC). In some embodiments, mesoporous silica particles comprise pores of between 2-50 nm in diameter. In some embodiments, the mesoporous silica particles have a surface area of at least about 100 m²/g. In some embodiments, the composition is suitable for injectable use. In some embodiments, the mesoporous silica particles are in the form of mesoporous silica rods.

In some embodiments, the viral vector is conjugated to first population of the mesoporous silica particles. In some embodiments, the viral vector is electrostatically or covalently conjugated to the first population of mesoporous silica particles. In some embodiments, the viral vector is a retrovirus, adenovirus, adeno-associated virus, herpes virus, or lentivirus. In some embodiments, the viral vector comprises an expression vector comprising a recombinant polynucleotide comprising an expression control sequence operatively linked to a nucleotide sequence to be expressed. In some embodiments, the nucleotide sequence encodes a chimeric antigen receptor (CAR), an engineered TCR, one or more cytokines, one or more chemokines, an shRNA to block an inhibitory molecule, or wherein the nucleotide sequence comprises an mRNA to induce expression of a protein. In some embodiments, the nucleotide sequence encodes a polypeptide engineered to target a tumor antigen. In some embodiments, the polypeptide targets a tumor antigen selected from the group consisting of: TSHR, CD19, CD123, CD22, CD30, CD171, CS-1, CLL-1, CD33, EGFRvIII, GD2, GD3, BCMA, Tn Ag, PSMA, ROR1, FLT3, FAP, TAG72, CD38, CD44v6, CEA, EPCAM, B7H3, KIT, IL-13Ra2, Mesothelin, IL-11Ra, PSCA, PRSS21, VEGFR2, LewisY, CD24, PDGFR-beta, SSEA-4, CD20, Folate receptor alpha, ERBB2 (Her2/neu), MUC1, EGFR, NCAM, Prostase, PAP, ELF2M, Ephrin B2, IGF-I receptor, CAIX, LMP2, gp100, bcr-abl, tyrosinase, EphA2, Fucosyl GM1, sLe, GM3, TGS5, HMWMAA, o-acetyl-GD2, Folate receptor beta, TEM1/CD248, TEM7R, CLDN6, GPRC5D, CXORF61, CD97, CD179a, ALK, Polysialic acid, PLAC1, GloboH, NY-BR-1, UPK2, HAVCR1, ADRB3, PANX3, GPR20, LY6K, OR51E2, TARP, WT1, NY-ESO-1, LAGE-1a, MAGE-A1, legumain, HPV E6, E7, MAGE A1, ETV6-AML, sperm protein 17, XAGE1, Tie 2, MAD-CT-1, MAD-CT-2, Fos-related antigen 1, p53, p53 mutant, prostein, survivin and telomerase, PCTA-1/Galectin 8, MelanA/MART1, Ras mutant, hTERT, sarcoma translocation breakpoints, ML-IAP, ERG (TMPRSS2 ETS fusion gene), NA17, PAX3, Androgen receptor, Cyclin B1, MYCN, RhoC, TRP-2, CYP1B1, BORIS, SART3, PAX5, OY-TES1, LCK, AKAP-4, SSX2, RAGE-1, human telomerase reverse transcriptase, RU1, RU2, intestinal carboxyl esterase, mut hsp70-2, CD79a, CD79b, CD72, LAIR1, FCAR, LILRA2, CD300LF, CLEC12A, BST2, EMR2, LY75, GPC3, FCRL5, IGLL1, and any combination thereof. In some embodiments, the protein is a CAR that comprises an antigen binding domain, a transmembrane domain, a costimulatory signaling region, and a signaling domain. In some embodiments, the signaling domain is a CD3 zeta signaling domain. In some embodiments, the costimulatory signaling region is selected from 41BB (i.e., CD137), CD27, ICOS, and/or CD28.

In some embodiments, the cell activation agent is conjugated to or absorbed on the first population of mesoporous silica particles or a second population of mesoporous silica particles. In some embodiments, the cell activation agent is a T cell stimulating compound, an anti-idiotype antibody to a CAR antigen binding domain, and/or a tumor antigen. In some embodiments, the T-cell stimulating compound is IL-2, IL-15, anti-CD2 mAb, anti-CD3 mAb, anti-CD28 mAb, neo-antigen peptides, peptides from shared antigens such as TRP2, gp100, tumor cell lysate, CD19, CD20, CD22, ROR1, mesothelin, CD33/IL3Ra, c-Met, PSMA, Glycolipid F77, EGFRvIII, GD-2, NY-ESO-1 TCR, and/or MAGE A3 TCR. In some embodiments, the cell activation agent comprises a CD3/TCR complex and/or an agent that stimulates a costimulatory molecule and/or growth factor receptor, optionally, wherein the cell activation agent is a multispecific binding molecule comprising an agent that stimulates a CD3/TCR complex and an agent that stimulates a costimulatory molecule and/or growth factor receptor. In some embodiments, the cell activation is selected from a sequence provided in Table 20. In some embodiments, the cell activation agent is conjugated to the second population of mesoporous silica particles or to a lipid envelope on the surface of the second population of mesoporous silica particles. In some embodiments, the composition further comprises a cytokine. In some embodiments, the cytokine is conjugated to or adsorbed on the first or second population of mesoporous silica particles. In some embodiments, the cytokine is IL-1, IL-2, IL-4, IL-5, IL-7, IL-10, IL-12, IL-15, IL-17, IL-21, or transforming growth factor beta (TGF-β), or an agonist thereof, a mimetic thereof, a variant thereof, a functional fragment thereof, or a combination thereof.

Also contemplated herein is a method of transducing cells in vivo comprising administering a biomaterial comprising a cell recruitment factor; a first population of mesoporous silica particles; a viral vector; and, optionally, a cell activation agent to a subject. In some embodiments, the components are administered simultaneously or sequentially. In some embodiments, the biomaterial comprising the cell recruitment factor is administered first. In some embodiments, the first population of mesoporous silica rods, the viral vector, and, optionally, the cell activation agent is administered simultaneously and, optionally, after the biomaterial.

Also contemplated herein is a method of transducing cells in vivo comprising administering a biomaterial and a cell recruitment factor; a first population of mesoporous silica particles; a viral vector; and, optionally, a cell activation agent to a subject. In some embodiments, the components are administered simultaneously or sequentially. In some embodiments, the biomaterial and the cell recruitment factor are administered first. In some embodiments, the first population of mesoporous silica rods, the viral vector, and, optionally, the cell activation agent is administered simultaneously and, optionally, after the biomaterial and the cell recruitment factor.

Also contemplated herein is a method of transducing cells in vivo comprising administering a biomaterial and a molecule (e.g., VEGF-C, IL-2, IL-7, IL-15 (e.g., hetIL-15 (IL15/sIL-15Ra)), GM-CSF, CXCL12, CXC3L1, CCL19, CCL21, CXCL10, or CXCL11); a first population of mesoporous silica particles; a viral vector; and, optionally, a cell activation agent to a subject. In some embodiments, the components are administered simultaneously or sequentially. In some embodiments, the biomaterial and the molecule are administered first. In some embodiments, the first population of mesoporous silica rods, the viral vector, and, optionally, the cell activation agent is administered simultaneously and, optionally, after the biomaterial and the molecule.

In some embodiments, the biomaterial comprises a hydrogel, optionally a cryogel. In some embodiments, the cryogel comprises gelatin, hyaluronic acid, collagen, alginate, laminin, chitosan, silk fibroin, agarose, poly(ethylene glycol), poly-vinyl alcohol, and/or hydroxyethyl methylacrylate. In some embodiments, the composition comprising the cryogel further comprises laponite. In some embodiments, the cryogel comprises pores between about 10 to 300 μm in diameter, optionally between about 50 to 300 μm. In some embodiments, the cryogel is chemically cross-linked.

In some embodiments, the cell recruitment factor selectively recruits immune cells, optionally T-cells and/or NK-cells. In some embodiments, the cell recruitment factor is selected from the group consisting of CCL19, CXCL9, CXCL10, XCL1, IL-2, IL-7, CCL21, GM-CSF, CCL17, CCL22, CCL20, CCL27, IL-15, lymphotoxin alpha, lymphotoxin beta, VEGF-C, FLT3L, G-CSF, PDGF, S100A8/A9, CSF-1, CXCL8, CCL20, CCL17, CCR5, CCR6, CCL2, VEGF, Angiopoietin-2, PGE2, LTB4, CXC3L1, CCL19, CCL21, CXCL10, CXCL11, and/or CXCL12. In some embodiments, the VEGF-C is selected from the group consisting of immature VEGF-C peptide or mature VEGF-C peptide. In some embodiments, the mature VEGF-C peptide is the minor mature form or major mature form. In some embodiments, the mature VEGF-C peptide is a wild type minor mature form or a wild type major mature form. In some embodiments, the mature VEGF-C peptide is a modified minor mature form or a modified major mature form. In some embodiments, the mature VEGF-C peptide is a modified minor mature form comprising a mutation at Cysteine 137 (e.g., C137A) or a modified major mature form comprising a mutation at Cysteine 137 (e.g., C137A). In some embodiments, the mature VEGF-C peptide is a modified minor mature form comprising a C137A mutation or a modified major mature form comprising a C137A mutation. In some embodiments, the mature VEGF-C peptide is present as a dimer or monomer. In some embodiments, the VEGF-C is a dimer of the major mature form further comprising a C137A mutation in each monomer. In some embodiments, the VEGF-C is a dimer of the minor mature form further comprising a C137A mutation in each monomer. In some embodiments, the VEGF-C is selected from Table 18, optionally, wherein the his tag is not included in the sequence. In some embodiments, the VEGF-C is present in an effective amount, optionally in an amount of less than or about 1 mg, less than or about 10 mg, greater than or about 10 μg, greater than or about 1 μg, between about 1 μg and 1 mg, between about 10 μg and 1 mg, between about 1 μg and 10 mg, or between about 10 μg and 10 mg.

In some embodiments, the first population of mesoporous silica particles are surface modified. In some embodiments, the surface modification on the first population of mesoporous silica particles is —OH (hydroxyl), amine, carboxylic acid, phosphonate, halide, azide, alkyne, epoxide, sulfhydryl, polyethyleneimine, a hydrophobic moiety, or salts thereof, optionally using a C₁ to C₂₀ alkyl or (—O(CH2-CH₂—)₁₋₂₅ linker. In some embodiments, the surface modification on the first population of mesoporous silica particles is a primary, secondary, tertiary, or quaternary amine. In some embodiments, the surface modification on the first population of mesoporous silica particles is a polyethyleneimine having an average molecular weight of about 1000 to 20,000 Da, about 1,200 to 15,000 Da, about 1,500 to 12,000 Da, about 2,000 Da, about 3,000 Da, about 4,000 Da, about 5,000 Da, about 6,000 Da, about 7,000 Da, about 8,000 Da, about 9,000 Da, or about 10,000 Da, as measured by gel permeation chromatography (GPC). In some embodiments, mesoporous silica particles comprise pores of between 2-50 nm in diameter. In some embodiments, the mesoporous silica particles have a surface area of at least about 100 m²/g. In some embodiments, the composition is suitable for injectable use. In some embodiments, the mesoporous silica particles are in the form of mesoporous silica rods.

In some embodiments, the viral vector is conjugated to first population of the mesoporous silica particles. In some embodiments, the viral vector is electrostatically or covalently conjugated to the first population of mesoporous silica particles. In some embodiments, the viral vector is a retrovirus, adenovirus, adeno-associated virus, herpes virus, or lentivirus. In some embodiments, the viral vector comprises an expression vector comprising a recombinant polynucleotide comprising an expression control sequence operatively linked to a nucleotide sequence to be expressed. In some embodiments, the nucleotide sequence encodes a chimeric antigen receptor (CAR), an engineered TCR, one or more cytokines, one or more chemokines, an shRNA to block an inhibitory molecule, or wherein the nucleotide sequence comprises an mRNA to induce expression of a protein. In some embodiments, the nucleotide sequence encodes a polypeptide engineered to target a tumor antigen. In some embodiments, the polypeptide targets a tumor antigen selected from the group consisting of: TSHR, CD19, CD123, CD22, CD30, CD171, CS-1, CLL-1, CD33, EGFRvIII, GD2, GD3, BCMA, Tn Ag, PSMA, ROR1, FLT3, FAP, TAG72, CD38, CD44v6, CEA, EPCAM, B7H3, KIT, IL-13Ra2, Mesothelin, IL-11Ra, PSCA, PRSS21, VEGFR2, LewisY, CD24, PDGFR-beta, SSEA-4, CD20, Folate receptor alpha, ERBB2 (Her2/neu), MUC1, EGFR, NCAM, Prostase, PAP, ELF2M, Ephrin B2, IGF-I receptor, CAIX, LMP2, gp100, bcr-abl, tyrosinase, EphA2, Fucosyl GM1, sLe, GM3, TGS5, HMWMAA, o-acetyl-GD2, Folate receptor beta, TEM1/CD248, TEM7R, CLDN6, GPRC5D, CXORF61, CD97, CD179a, ALK, Polysialic acid, PLAC1, GloboH, NY-BR-1, UPK2, HAVCR1, ADRB3, PANX3, GPR20, LY6K, OR51E2, TARP, WT1, NY-ESO-1, LAGE-1a, MAGE-A1, legumain, HPV E6, E7, MAGE A1, ETV6-AML, sperm protein 17, XAGE1, Tie 2, MAD-CT-1, MAD-CT-2, Fos-related antigen 1, p53, p53 mutant, prostein, survivin and telomerase, PCTA-1/Galectin 8, MelanA/MART1, Ras mutant, hTERT, sarcoma translocation breakpoints, ML-IAP, ERG (TMPRSS2 ETS fusion gene), NA17, PAX3, Androgen receptor, Cyclin B1, MYCN, RhoC, TRP-2, CYP1B1, BORIS, SART3, PAX5, OY-TES1, LCK, AKAP-4, SSX2, RAGE-1, human telomerase reverse transcriptase, RU1, RU2, intestinal carboxyl esterase, mut hsp70-2, CD79a, CD79b, CD72, LAIR1, FCAR, LILRA2, CD300LF, CLEC12A, BST2, EMR2, LY75, GPC3, FCRL5, IGLL1, and any combination thereof. In some embodiments, the protein is a CAR that comprises an antigen binding domain, a transmembrane domain, a costimulatory signaling region, and a signaling domain. In some embodiments, the signaling domain is a CD3 zeta signaling domain. In some embodiments, the costimulatory signaling region is selected from 41BB (i.e., CD137), CD27, ICOS, and/or CD28.

In some embodiments, the cell activation agent is conjugated to or absorbed on the first population of mesoporous silica particles or a second population of mesoporous silica particles. In some embodiments, the cell activation agent is a T cell stimulating compound, an anti-idiotype antibody to a CAR antigen binding domain, and/or a tumor antigen. In some embodiments, the T-cell stimulating compound is IL-2, IL-15, anti-CD2 mAb, anti-CD3 mAb, anti-CD28 mAb, neo-antigen peptides, peptides from shared antigens such as TRP2, gp100, tumor cell lysate, CD19, CD20, CD22, ROR1, mesothelin, CD33/IL3Ra, c-Met, PSMA, Glycolipid F77, EGFRvIII, GD-2, NY-ESO-1 TCR, and/or MAGE A3 TCR. In some embodiments, the cell activation agent comprises a CD3/TCR complex and/or an agent that stimulates a costimulatory molecule and/or growth factor receptor, optionally, wherein the cell activation agent is a multispecific binding molecule comprising an agent that stimulates a CD3/TCR complex and an agent that stimulates a costimulatory molecule and/or growth factor receptor. In some embodiments, the cell activation is selected from a sequence provided in Table 20. In some embodiments, the cell activation agent is conjugated to the second population of mesoporous silica particles or to a lipid envelope on the surface of the second population of mesoporous silica particles. In some embodiments, the composition further comprises a cytokine. In some embodiments, the cytokine is conjugated to or adsorbed on the first or second population of mesoporous silica particles. In some embodiments, the cytokine is IL-1, IL-2, IL-4, IL-5, IL-7, IL-10, IL-12, IL-15, IL-17, IL-21, or transforming growth factor beta (TGF-β), or an agonist thereof, a mimetic thereof, a variant thereof, a functional fragment thereof, or a combination thereof.

Also contemplated herein is a method of treating a subject having a disease, disorder, or condition comprising administering a biomaterial comprising a cell recruitment factor; a first population of mesoporous silica particles; a viral vector; and, optionally, a cell activation agent to a subject. In some embodiments, the components are administered simultaneously or sequentially. In some embodiments, the biomaterial comprising the cell recruitment factor is administered first. In some embodiments, the first population of mesoporous silica rods, the viral vector, and, optionally, the cell activation agent is administered simultaneously and, optionally, after the biomaterial.

Also contemplated herein is a method of treating a subject having a disease, disorder, or condition comprising administering a biomaterial and a cell recruitment factor; a first population of mesoporous silica particles; a viral vector; and, optionally, a cell activation agent to a subject. In some embodiments, the components are administered simultaneously or sequentially. In some embodiments, the biomaterial and the cell recruitment factor are administered first. In some embodiments, the first population of mesoporous silica rods, the viral vector, and, optionally, the cell activation agent is administered simultaneously and, optionally, after the biomaterial and the cell recruitment factor.

Also contemplated herein is a method of treating a subject having a disease, disorder, or condition comprising administering a biomaterial and a molecule (e.g., VEGF-C, IL-2, IL-7, IL-15 (e.g., hetIL-15 (IL15/sIL-15Ra)), GM-CSF, CXCL12, CXC3L1, CCL19, CCL21, CXCL10, or CXCL11); a first population of mesoporous silica particles; a viral vector; and, optionally, a cell activation agent to a subject. In some embodiments, the components are administered simultaneously or sequentially. In some embodiments, the biomaterial and the molecule are administered first. In some embodiments, the first population of mesoporous silica rods, the viral vector, and, optionally, the cell activation agent is administered simultaneously and, optionally, after the biomaterial and the molecule.

In some embodiments, the subject has cancer. In some embodiments, the subject has cancer expressing one or more tumor antigen selected from the group consisting of: TSHR, CD19, CD123, CD22, CD30, CD171, CS-1, CLL-1, CD33, EGFRvIII, GD2, GD3, BCMA, Tn Ag, PSMA, ROR1, FLT3, FAP, TAG72, CD38, CD44v6, CEA, EPCAM, B7H3, KIT, IL-13Ra2, Mesothelin, IL-11Ra, PSCA, PRSS21, VEGFR2, LewisY, CD24, PDGFR-beta, SSEA-4, CD20, Folate receptor alpha, ERBB2 (Her2/neu), MUC1, EGFR, NCAM, Prostase, PAP, ELF2M, Ephrin B2, IGF-I receptor, CAIX, LMP2, gp100, ber-abl, tyrosinase, EphA2, Fucosyl GM1, sLe, GM3, TGS5, HMWMAA, o-acetyl-GD2, Folate receptor beta, TEM1/CD248, TEM7R, CLDN6, GPRC5D, CXORF61, CD97, CD179a, ALK, Polysialic acid, PLAC1, GloboH, NY-BR-1, UPK2, HAVCR1, ADRB3, PANX3, GPR20, LY6K, OR51E2, TARP, WT1, NY-ESO-1, LAGE-1a, MAGE-A1, legumain, HPV E6, E7, MAGE A1, ETV6-AML, sperm protein 17, XAGE1, Tie 2, MAD-CT-1, MAD-CT-2, Fos-related antigen 1, p53, p53 mutant, prostein, survivin and telomerase, PCTA-1/Galectin 8, MelanA/MART1, Ras mutant, hTERT, sarcoma translocation breakpoints, ML-IAP, ERG (TMPRSS2 ETS fusion gene), NA17, PAX3, Androgen receptor, Cyclin B1, MYCN, RhoC, TRP-2, CYP1B1, BORIS, SART3, PAX5, OY-TES1, LCK, AKAP-4, SSX2, RAGE-1, human telomerase reverse transcriptase, RU1, RU2, intestinal carboxyl esterase, mut hsp70-2, CD79a, CD79b, CD72, LAIR1, FCAR, LILRA2, CD300LF, CLEC12A, BST2, EMR2, LY75, GPC3, FCRL5, IGLL1, and any combination thereof.

In some embodiments, the biomaterial comprises a hydrogel, optionally a cryogel. In some embodiments, the cryogel comprises gelatin, hyaluronic acid, collagen, alginate, laminin, chitosan, silk fibroin, agarose, poly(ethylene glycol), poly-vinyl alcohol, and/or hydroxyethyl methylacrylate. In some embodiments, the composition comprising the cryogel further comprises laponite. In some embodiments, the cryogel comprises pores between about 10 to 300 μm in diameter, optionally between about 50 to 300 μm. In some embodiments, the cryogel is chemically cross-linked.

In some embodiments, the cell recruitment factor selectively recruits immune cells, optionally T-cells and/or NK-cells. In some embodiments, the cell recruitment factor is selected from the group consisting of CCL19, CXCL9, CXCL10, XCL1, IL-2, IL-7, CCL21, GM-CSF, CCL17, CCL22, CCL20, CCL27, IL-15, lymphotoxin alpha, lymphotoxin beta, VEGF-C, FLT3L, G-CSF, PDGF, S100A8/A9, CSF-1, CXCL8, CCL20, CCL17, CCR5, CCR6, CCL2, VEGF, Angiopoietin-2, PGE2, LTB4, CXC3L1, CCL19, CCL21, CXCL10, CXCL11, and/or CXCL12. In some embodiments, the VEGF-C is selected from the group consisting of immature VEGF-C propeptide or mature VEGF-C peptide. In some embodiments, the mature VEGF-C peptide is the minor mature form or major mature form. In some embodiments, the mature VEGF-C peptide is a wild type minor mature form or a wild type major mature form. In some embodiments, the mature VEGF-C peptide is a modified minor mature form or a modified major mature form. In some embodiments, the mature VEGF-C peptide is a modified minor mature form comprising a mutation at Cysteine 137 (e.g., C137A) or a modified major mature form comprising a mutation at Cysteine 137 (e.g., C137A). In some embodiments, the mature VEGF-C peptide is a modified minor mature form comprising a C137A mutation or a modified major mature form comprising a C137A mutation. In some embodiments, the mature VEGF-C peptide is present as a dimer or monomer. In some embodiments, the VEGF-C is a dimer of the major mature form further comprising a C137A mutation in each monomer. In some embodiments, the VEGF-C is a dimer of the minor mature form further comprising a C137A mutation in each monomer. In some embodiments, the VEGF-C is selected from Table 18, optionally, wherein the his tag is not included in the sequence. In some embodiments, the VEGF-C is present in an effective amount, optionally in an amount of less than or about 1 mg, less than or about 10 mg, greater than or about 10 μg, greater than or about 1 μg, between about 1 μg and 1 mg, between about 10 μg and 1 mg, between about 1 μg and 10 mg, or between about 10 μg and 10 mg.

In some embodiments, the first population of mesoporous silica particles are surface modified. In some embodiments, the surface modification on the first population of mesoporous silica particles is —OH (hydroxyl), amine, carboxylic acid, phosphonate, halide, azide, alkyne, epoxide, sulfhydryl, polyethyleneimine, a hydrophobic moiety, or salts thereof, optionally using a C₁ to C₂₀ alkyl or (—O(CH2-CH₂—)₁₋₂₅ linker. In some embodiments, the surface modification on the first population of mesoporous silica particles is a primary, secondary, tertiary, or quaternary amine. In some embodiments, the surface modification on the first population of mesoporous silica particles is a polyethyleneimine having an average molecular weight of about 1000 to 20,000 Da, about 1,200 to 15,000 Da, about 1,500 to 12,000 Da, about 2,000 Da, about 3,000 Da, about 4,000 Da, about 5,000 Da, about 6,000 Da, about 7,000 Da, about 8,000 Da, about 9,000 Da, or about 10,000 Da, as measured by gel permeation chromatography (GPC). In some embodiments, mesoporous silica particles comprise pores of between 2-50 nm in diameter. In some embodiments, the mesoporous silica particles have a surface area of at least about 100 m²/g. In some embodiments, the composition is suitable for injectable use. In some embodiments, the mesoporous silica particles are in the form of mesoporous silica rods.

In some embodiments, the viral vector is conjugated to first population of the mesoporous silica particles. In some embodiments, the viral vector is electrostatically or covalently conjugated to the first population of mesoporous silica particles. In some embodiments, the viral vector is a retrovirus, adenovirus, adeno-associated virus, herpes virus, or lentivirus. In some embodiments, the viral vector comprises an expression vector comprising a recombinant polynucleotide comprising an expression control sequence operatively linked to a nucleotide sequence to be expressed. In some embodiments, the nucleotide sequence encodes a chimeric antigen receptor (CAR), an engineered TCR, one or more cytokines, one or more chemokines, an shRNA to block an inhibitory molecule, or wherein the nucleotide sequence comprises an mRNA to induce expression of a protein. In some embodiments, the nucleotide sequence encodes a polypeptide engineered to target a tumor antigen. In some embodiments, the polypeptide targets a tumor antigen selected from the group consisting of: TSHR, CD19, CD123, CD22, CD30, CD171, CS-1, CLL-1, CD33, EGFRvIII, GD2, GD3, BCMA, Tn Ag, PSMA, ROR1, FLT3, FAP, TAG72, CD38, CD44v6, CEA, EPCAM, B7H3, KIT, IL-13Ra2, Mesothelin, IL-11Ra, PSCA, PRSS21, VEGFR2, LewisY, CD24, PDGFR-beta, SSEA-4, CD20, Folate receptor alpha, ERBB2 (Her2/neu), MUC1, EGFR, NCAM, Prostase, PAP, ELF2M, Ephrin B2, IGF-I receptor, CAIX, LMP2, gp100, bcr-abl, tyrosinase, EphA2, Fucosyl GM1, sLe, GM3, TGS5, HMWMAA, o-acetyl-GD2, Folate receptor beta, TEM1/CD248, TEM7R, CLDN6, GPRC5D, CXORF61, CD97, CD179a, ALK, Polysialic acid, PLAC1, GloboH, NY-BR-1, UPK2, HAVCR1, ADRB3, PANX3, GPR20, LY6K, OR51E2, TARP, WT1, NY-ESO-1, LAGE-1a, MAGE-A1, legumain, HPV E6, E7, MAGE A1, ETV6-AML, sperm protein 17, XAGE1, Tie 2, MAD-CT-1, MAD-CT-2, Fos-related antigen 1, p53, p53 mutant, prostein, survivin and telomerase, PCTA-1/Galectin 8, MelanA/MART1, Ras mutant, hTERT, sarcoma translocation breakpoints, ML-IAP, ERG (TMPRSS2 ETS fusion gene), NA17, PAX3, Androgen receptor, Cyclin B1, MYCN, RhoC, TRP-2, CYP1B1, BORIS, SART3, PAX5, OY-TES1, LCK, AKAP-4, SSX2, RAGE-1, human telomerase reverse transcriptase, RU1, RU2, intestinal carboxyl esterase, mut hsp70-2, CD79a, CD79b, CD72, LAIR1, FCAR, LILRA2, CD300LF, CLEC12A, BST2, EMR2, LY75, GPC3, FCRL5, IGLL1, and any combination thereof. In some embodiments, the protein is a CAR that comprises an antigen binding domain, a transmembrane domain, a costimulatory signaling region, and a signaling domain. In some embodiments, the signaling domain is a CD3 zeta signaling domain. In some embodiments, the costimulatory signaling region is selected from 41BB (i.e., CD137), CD27, ICOS, and/or CD28. In some embodiments, the cell activation agent is conjugated to or absorbed on the first population of mesoporous silica particles or a second population of mesoporous silica particles. In some embodiments, the cell activation agent is a T cell stimulating compound, an anti-idiotype antibody to a CAR antigen binding domain, and/or a tumor antigen. In some embodiments, the T-cell stimulating compound is IL-2, IL-15, anti-CD2 mAb, anti-CD3 mAb, anti-CD28 mAb, neo-antigen peptides, peptides from shared antigens such as TRP2, gp100, tumor cell lysate, CD19, CD20, CD22, ROR1, mesothelin, CD33/IL3Ra, c-Met, PSMA, Glycolipid F77, EGFRvIII, GD-2, NY-ESO-1 TCR, and/or MAGE A3 TCR. In some embodiments, the cell activation agent comprises a CD3/TCR complex and/or an agent that stimulates a costimulatory molecule and/or growth factor receptor, optionally, wherein the cell activation agent is a multispecific binding molecule comprising an agent that stimulates a CD3/TCR complex and an agent that stimulates a costimulatory molecule and/or growth factor receptor. In some embodiments, the cell activation is selected from a sequence provided in Table 20. In some embodiments, the cell activation agent is conjugated to the second population of mesoporous silica particles or to a lipid envelope on the surface of the second population of mesoporous silica particles. In some embodiments, the composition further comprises a cytokine. In some embodiments, the cytokine is conjugated to or adsorbed on the first or second population of mesoporous silica particles. In some embodiments, the cytokine is IL-1, IL-2, IL-4, IL-5, IL-7, IL-10, IL-12, IL-15, IL-17, IL-21, or transforming growth factor beta (TGF-β), or an agonist thereof, a mimetic thereof, a variant thereof, a functional fragment thereof, or a combination thereof.

Those skilled in the art will recognize or be able to ascertain using no more than routine experimentation, many equivalents to the specific embodiments of the invention described herein. Such equivalents are intended to be encompassed by the following enumerated embodiments.

### Enumerated Embodiments

1. A composition, comprising:
(a) biomaterial comprising a cell recruitment factor;
(b) a first population of mesoporous silica particles;
(c) a viral vector; and
(d) optionally, a cell activation agent.

2. The composition of embodiment 1, wherein the biomaterial comprises a hydrogel, optionally a cryogel.

3. The composition of embodiment 2, wherein the cryogel:
(a) comprises gelatin, hyaluronic acid, collagen, alginate, laminin, chitosan, silk fibroin, agarose, poly(ethylene glycol), poly-vinyl alcohol, and/or hydroxyethyl methylacrylate, optionally, wherein the cryogel further comprises laponite;
(b) comprises pores between about 10 to 300 μm in diameter, optionally between about 50 to 300 μm; and/or
(c) is chemically cross-linked.

4. The composition of any one of the preceding embodiments, wherein the cell recruitment factor is selected from the group consisting of CCL19, CXCL9, CXCL10, XCL1, IL-2, IL-7, CCL21, GM-CSF, CCL17, CCL22, CCL20, CCL27, IL-15, lymphotoxin alpha, lymphotoxin beta, VEGF-C, FLT3L, G-CSF, PDGF, S100A8/A9, CSF-1, CXCL8, CCL20, CCL17, CCR5, CCR6, CCL2, VEGF, Angiopoietin-2, PGE2, LTB4, CXC3L1, CCL19, CCL21, CXCL10, CXCL 11, and/or CXCL12.

5. The composition of any one of the preceding embodiments, wherein the cell recruitment factor selectively recruits immune cells, optionally T-cells and/or NK-cells.

6. The composition of embodiment 5, wherein the cell recruitment factor is selected from the group consisting of:
(a) IL-2, IL-7, CCL21, IL-15, GM-CSF, and/or VEGF-C for the recruitment of T-cells; and/or
(b) CXCL12, CXC3L1, CCL19, CCL21, CXCL10, and/or CXCL11 for the recruitment of NK-cells.

7. The composition of any one of the preceding embodiments, wherein the cell recruitment factor is VEGF-C, optionally as a monomer or a dimer.

8. The composition of embodiment 7, wherein the VEGF-C is a mature VEGF-C peptide, optionally the minor or major mature form or a mutated variant each thereof.

9. The composition of embodiment 8, wherein the mature VEGF-C peptide comprises a C137A mutation.

10. The composition of any one of embodiments 7 to 9, wherein the VEGF-C comprises a sequence provided in Table 18, optionally, wherein the his tag is not included in the sequence.

11. The composition of embodiment 10, wherein the VEGF-C comprises a dimer of one or more of a sequence provided in Table 18, optionally, wherein the his tag is not included in the sequence.

12. The composition of any one of embodiments 7 to 10, wherein the VEGF-C is present in an effective amount, optionally, in an amount of less than or about 1 mg, less than or about 10 mg, greater than or about 10 μg, greater than or about 1 μg, between about 1 μg and 1 mg, between about 10 μg and 1 mg, between about 1 μg and 10 mg, or between about 10 μg and 10 mg.

13. The composition of any one of the preceding embodiments, wherein the viral vector is conjugated to the first population of mesoporous silica particles.

14. The composition of embodiment 13, wherein the viral vector is electrostatically or covalently conjugated to the first population of mesoporous silica particles.

15. The composition of any one of the preceding embodiments, wherein the first population of mesoporous silica particles are surface modified.

16. The composition of embodiment 15, wherein the surface modification on the first population of mesoporous silica particles is —OH (hydroxyl), amine, carboxylic acid, phosphonate, halide, azide, alkyne, epoxide, sulfhydryl, polyethyleneimine, a hydrophobic moiety, or salts thereof, optionally using a C₁ to C₂₀ alkyl or (—O(CH2-CH₂—)₁₋₂₅ linker.

17. The composition of embodiment 15, wherein the surface modification on the first population of mesoporous silica particles is a primary, secondary, tertiary, or quaternary amine.

18. The composition of embodiment 15, wherein the surface modification on the first population of mesoporous silica particles is a polyethyleneimine having an average molecular weight of about 1000 to 20,000 Da, about 1,200 to 15,000 Da, about 1,500 to 12,000 Da, about 2,000 Da, about 3,000 Da, about 4,000 Da, about 5,000 Da, about 6,000 Da, about 7,000 Da, about 8,000 Da, about 9,000 Da, or about 10,000 Da, as measured by gel permeation chromatography (GPC).

19. The composition of any one of the preceding embodiments, wherein the viral vector is a retrovirus, adenovirus, adeno-associated virus, herpes virus, or lentivirus.

20. The composition of any one of the preceding embodiments, wherein the viral vector comprises an expression vector comprising a recombinant polynucleotide comprising an expression control sequence operatively linked to a nucleotide sequence to be expressed.

21. The composition of embodiment 20, wherein the nucleotide sequence encodes a chimeric antigen receptor (CAR), an engineered TCR, one or more cytokines, one or more chemokines, an shRNA to block an inhibitory molecule, or wherein the nucleotide sequence comprises an mRNA to induce expression of a protein.

22. The composition of embodiment 21, wherein the nucleotide sequence encodes a polypeptide engineered to target a tumor antigen.

23. The composition of embodiment 22, wherein the tumor antigen selected from the group consisting of: TSHR, CD19, CD123, CD22, CD30, CD171, CS-1, CLL-1, CD33, EGFRvIII, GD2, GD3, BCMA, Tn Ag, PSMA, ROR1, FLT3, FAP, TAG72, CD38, CD44v6, CEA, EPCAM, B7H3, KIT, IL-13Ra2, Mesothelin, IL-11Ra, PSCA, PRSS21, VEGFR2, LewisY, CD24, PDGFR-beta, SSEA-4, CD20, Folate receptor alpha, ERBB2 (Her2/neu), MUC1, EGFR, NCAM, Prostase, PAP, ELF2M, Ephrin B2, IGF-I receptor, CAIX, LMP2, gp100, bcr-abl, tyrosinase, EphA2, Fucosyl GM1, sLe, GM3, TGS5, HMWMAA, o-acetyl-GD2, Folate receptor beta, TEM1/CD248, TEM7R, CLDN6, GPRC5D, CXORF61, CD97, CD179a, ALK, Polysialic acid, PLAC1, GloboH, NY-BR-1, UPK2, HAVCR1, ADRB3, PANX3, GPR20, LY6K, OR51E2, TARP, WT1, NY-ESO-1, LAGE-1a, MAGE-A1, legumain, HPV E6, E7, MAGE A1, ETV6-AML, sperm protein 17, XAGE1, Tie 2, MAD-CT-1, MAD-CT-2, Fos-related antigen 1, p53, p53 mutant, prostein, survivin and telomerase, PCTA-1/Galectin 8, MelanA/MART1, Ras mutant, hTERT, sarcoma translocation breakpoints, ML-IAP, ERG (TMPRSS2 ETS fusion gene), NA17, PAX3, Androgen receptor, Cyclin B1, MYCN, RhoC, TRP-2, CYP1B1, BORIS, SART3, PAX5, OY-TES1, LCK, AKAP-4, SSX2, RAGE-1, human telomerase reverse transcriptase, RU1, RU2, intestinal carboxyl esterase, mut hsp70-2, CD79a, CD79b, CD72, LAIR1, FCAR, LILRA2, CD300LF, CLEC12A, BST2, EMR2, LY75, GPC3, FCRL5, IGLL1, and any combination thereof.

24. The composition of any one of the preceding embodiments, wherein the vector encodes a CAR that comprises an antigen binding domain, a transmembrane domain, a costimulatory signaling region, and a signaling domain.

25. The composition of embodiment 24, wherein:
(a) the antigen binding domain binds an antigen selected from CD19, CD123, CD22, CD20, EGFRvIII, BCMA, Mesothelin, CD33, CLL-1, and any combination thereof,
(b) the transmembrane domain comprises a CD8 hinge;
(c) the costimulatory signaling region is selected from a 4-1BB or CD28 costimulatory signaling domain; and/or
(d) the signaling region comprises a CD3 zeta signaling domain.

26. The composition of any one of the preceding embodiments, wherein the cell activation agent is a T cell stimulating compound, an anti-idiotype antibody to a CAR antigen binding domain, or the tumor antigen.

27. The composition of any one of the preceding embodiments, wherein the cell activation agent:
(a) comprises a CD3/TCR complex and/or an agent that stimulates a costimulatory molecule and/or growth factor receptor;
(b) is a multispecific binding molecule comprising n agent that stimulates a CD3/TCR complex and an agent that stimulates a costimulatory molecule and/or growth factor receptor; and/or
(c) comprises a sequence provided in Table 20 and/or provided in one or more format according to FIG. 37.

28. The composition of any one of the preceding embodiments, wherein the cell activation agent is conjugated to or adsorbed on the first population of mesoporous silica particles, a second population of mesoporous silica particles, or to a lipid envelope on the surface of the second population of mesoporous silica particles.

29. The composition of any one of the preceding embodiments, wherein the mesoporous silica particles are mesoporous silica rods.

30. The composition of any of the preceding embodiments, wherein the mesoporous silica particles comprise pores of between 2-50 nm in diameter.

31. The composition of any of the preceding embodiments, wherein the mesoporous silica particles have a surface area of at least about 100 m²/g.

32. The composition of any of the preceding embodiments, wherein the composition is suitable for injectable use.

33. A method of transducing cells in vivo comprising administering the composition of any one of embodiments 1 to 32, each component being administered simultaneously or sequentially.

34. A method of treating a disease, disorder, or condition comprising administering the composition of any one of embodiments 1 to 32, each component being administered simultaneously or sequentially.

35. The method of embodiment 34, wherein the disease, disorder, or condition is cancer.

### BRIEF DESCRIPTION OF THE DRAWINGS

FIG. 1 presents a series of surface modifications on mesoporous silica particles.

FIG. 2 presents results from staining for viral envelope protein (VSV-G) on MSR surface after adsorption of VSV-G pseudotyped lentivirus onto MSRs. The control MSRs are presented on the top panel and virus-incubated rods are on the bottom panel.

FIG. 3 is a schematic of virus adsorption on MSRs and transduction of T cells.

FIG. 4 provides results from GFP expression by T cells incubated with free lentivirus or MSR-bound lentivirus. Dilution of virus-coated MSRs from 40 μg/ml starting concentration is as indicated. The “1× lenti” condition is equivalent to the amount of virus incubated with the MSR conditions. The “2× lenti” condition is equivalent to twice the amount used to coat the MSR conditions.

FIG. 5 provides a schematic of overall strategy for ligand presentation on MSR surface. Liposomes are incubated with MSRs to form a supported lipid bilayer. Ligands can be coupled to the MSR-lipid bilayer using streptavidin-biotin interactions.

FIG. 6 shows a picture of MSRs coated with POPC liposomes containing 1 mol % PE-carboxyfluorescein. Bright field (left), fluorescence (middle), and overlay (right) images are shown.

FIG. 7 depicts the peptide sequence of EGFRvIII CAR-binding peptide (LEEKKGNYVVTDH (SEQ ID NO: 756)).

FIG. 8 illustrates cytokine production of EGFRvIII CARTs by peptide immobilization on MSRs. Results provide interferon-gamma and interleukin-2 production of EGFRvIII CARTs stimulated by lipid-coated MSRs (1% PE-biotin in the lipid coating) presenting EGFRvIII-CAR binding peptide compared to control conditions control conditions.

FIG. 9 illustrates the proliferation of EGFRvIII CARTs by peptide immobilization on MSRs. A lipid-coated MSR composition of 0.01% PE-biotin was used for peptide immobilization, and the MSR concentration was 30 μg/ml in the well. Cell counts were performed at day 7 of culture under the indicated conditions.

FIGS. 10A and 10B illustrate the proliferation of EGFRvIII CARTs and final cellular composition by peptide immobilization on MSRs. The starting MSR concentration was 50 μg/ml with and the dilutions of MSRs from this starting concentration are as indicated in the axis. FIG. 10A shows the percentage of CD4 and CD8 T cells at the end of culture period with the indicated materials. FIG. 10B depicts the FACS analysis of CD8+ and CD4+ CAR T cells diluting CFSE during a 3-day culture period using MSRs with varying amount of EGFRvIII CAR-binding peptide with or without anti-CD28 on the MSR surface.

FIGS. 11A and 11B illustrate the proliferation of BCMA CARTs and final cellular composition by BCMA protein immobilization on MSRs. The starting MSR concentration was 50 μg/ml with and the dilutions of MSRs from this starting concentration are as indicated in the axis. FIG. 11A shows percentage of CD4 and CD8 T cells at the end of culture period with the indicated materials. FIG. 11B demonstrates FACS analysis of CD8+ and CD4+ CAR T cells diluting CFSE during a 3-day culture period using MSRs with varying amount of EGFRvIII CAR-binding peptide with or without anti-CD28 on the MSR surface.

FIG. 12 presents a schematic of simultaneous stimulation and transduction of unstimulated human T cells using MSRs, according to some embodiments. Two populations of MSRs are created—1) MSRs presenting agonistic CD3/CD28 antibodies to stimulate T cells, 2) Positively charged PEI-MSRs that have been bound with lentivirus to facilitate viral delivery to the T cells. The two types of MSRs can be mixed together in different ratios to adjust the amount of stimulation and virus that the T cells are exposed to.

FIG. 13 illustrates the transduction efficiency of T cells exposed to stimulatory (anti-CD3/CD28 antibody-immobilized MSRs) and PEI-MSRs incubated with virus. T cells were incubated with different amounts of stimulating rods (Stim 1.00 represents 70 μg/ml MSRs) and exposed to GFP-lentivirus at different multiplicities of infection (MOI) either bound to PEI-MSRs or in free virus form. The top concentration of MSRs in the virus conditions was 22 μg/ml.

FIG. 14 illustrates transduction efficiency of T cells exposed to stimulatory (anti-CD3/CD28 antibody-immobilized) MSRs and PEI-MSRs incubated with virus. Plots show transduction efficiency as a function of the concentration of stimulatory MSRs at various total amounts of virus. The MSR concentration of stimulating MSR condition 1.0 is 70 μg/ml. The concentration of MSRs in the PEI MSR condition 1 is 22 μg/ml. Transduction was assessed at 3 days after initiation of the culture.

FIG. 15 provides results from comparison of virus delivery strategies for transduction efficiency. In the “PEI” and “free” conditions T cells were stimulated with a “high” level of CD3/CD28 antibodies bound to MSRs (MSR concentration 70 μg/ml), and given virus either associated with PEI-MSRs or freely delivered in the media, respectively (virus concentration 1.0 contains 22 μg/ml MSRs, MOI ˜6.7). In the “PEI+CD3/CD28” the virus and CD3/CD28 agonistic antibodies were bound to PEI-MSRs (concentration 1.0 is 22 μg/ml MSRs). Transduction was assessed at 3 days after initiation of the culture.

FIG. 16 provides results from comparison of various delivery strategies for transduction in PBMC population. Conditions as in FIG. 15 were added to PBMCs. The proportion of transduced cells in each cell type was quantified. Transduction was assessed at 3 days after initiation of the culture.

FIG. 17 provides the different transduction fractions in PBMCs with various virus delivery strategies. Top panel provides the total cell composition present in PBMC populations under the conditions of FIG. 15. Bottom panel provides the composition of the transduced cell fraction present after virus delivery using the conditions of FIG. 15. Transduction was assessed at 3 days after initiation of the culture.

FIG. 18 provides a non-limiting exemplary schematic of the composition disclosed herein for use as a cancer therapeutic wherein (A) depicts delivering the growth factor VEGF-C that induces lymphangiogenesis of pre-existing skin lymphatic capillaries; (B) depicts the generated lymphatic endothelial cells secrete chemokines (such as CCL21) that attract immune cells (primarily naive T cells) from the blood circulation into the dermis on top of the gel; (C) depicts lymphatic endothelial cells educating T cells—not to be bound by theory, Applicants believe that such education could yield a high proportion of stem cell memory phenotype, which in turn may be more potent cells once transduced; after T cells are recruited to this priming site, (D) depicts lentivirus encoding for one or more chimeric antigen receptors (CARs) being delivered in combination with mesoporous silica rods to prevent systemic spread—not to be bound by theory, Applicants believe that local positioning of the virus could favor transduction of locally recruited T cells and that activation could further improve transduction, prevent leakage, and transduction of unwanted cells; (E) and (F) depict transduced and educated cells returning into systemic circulation via the skin lymphatics, lymph nodes and thoracic duct—not to be bound by theory, Applicants believe that such cells will further expand in response to tumor antigen.

FIGS. 19A-19C lay out the characteristics of various VEGF-C protein variants. FIG. 19A provides a schematic of both natural and modified VEGF-C. Immature VEGF-C(#1) is normally found intracellularly with N-terminal and C-terminal propeptide sequences. Once released, VEGF-C protein undergoes proteolytic cleavage and is present in the extracellular space as minor or major mature form (#2, #7) in dimer or monomer form. Stabilized dimers of major (#9) and minor mature (#8) form were engineered by inserting the C137A mutation into the sequences. The main difference between minor mature and major mature forms is the presence of an additional short propeptide sequence on the minor mature form N-terminus. Not to be bound by theory, Applicants believe the additional short propeptide appears to boost dimer formation as well as protein expression in both HEK293T and CHO MaKo cells. FIG. 19B depicts an SDS-polyacrylamide gel subjected to electrophoresis under non-reducing (NR) and reducing conditions (R) loaded with purified VEGF-C variants. Details of the wells with the corresponding VEGF-C variant are shown in FIG. 19C and presence of dimer (**) and monomer (*) have been indicated with asterix. The immature VEGF-C with the full length propepitde (#1) is produced as a dimer, among some impurities (wells 2 and 3); the removal of the propeptide leads to the production of two major mature VEGF-C forms (#2), one non-covalent dimer form (wells 5, 6) and a monomeric form (wells 8, 9); adding the mutation C137A in #2 leads to the production of a covalent dimer (well 21, 22) and a monomer form (well 24, 25). A short N-terminal propeptide is left attached to the protein to generate a minor mature VEGF-C form (#7) in non-covalent dimer (wells 11,12) or in monomeric form (wells 15,16). Not to be bound by theory, Applicants have determined that the C137A mutation to #7 VEGF-C leads to the generation of VEGF-C dimer only (wells 18,19) which may be suitable for large scale production. Rows 1, 4, 7, 10, 13, 14, 17, 20, 23 and 26 show the molecular weight marker in kDa.

FIGS. 20A-20C show the results of HDLECs sprouting assay with various VEGF-C variants. Dimeric VEGF-C forms appeared to demonstrate good in vitro activity. FIG. 20A depicts the experimental setup of in vitro sprouting assay on human dermal lymphatic endothelial cells (HDLECs) to test biological activity of VEGF-C variants. After incubation cells were FIG. 20B assessed for proliferation by WT-8 assay and FIG. 20C imaged for tube formation after phalloidin staining. Tube formation images show that the major and minor mature forms (#2, 7, 8, 9) stimulate better sprouting of HDLECs than immature form (#1). Not to be bound by theory, it is believed that the dimeric forms (#2D,7D,8D,9D) superior sprouting activity renders them preferable to monomeric forms (#2M,7M,9M).

FIGS. 21A-21D show the release rate of VEGF-C from alginate cryogel formulations, as well as proof of concept that it can be injected subcutaneously in a mouse. FIG. 21A provides a non-limiting, exemplary schematic of alginate cryogels manufacturing. Alginate liquid prepolymer is mixed with laponite and protein of interest, then frozen and thawed again before injection to generate porous matrix. FIG. 21B shows the modulation of VEGF-C release in vitro by different types of alginate gels: alginate nanoporous (gelification occurs before cryogelation so no pores are formed), alginate cryogel (after freeze and cryogelation, porous), and alginate cryogel with 0.25% laponite. 0.25% laponite alginate cryogel is showing a controlled and long-lasting release of VEGF-C protein in vitro. FIG. 21C shows the modulation of VEGF-C release profile in vitro from 0.25% or 0.5% laponite alginate cryogels loaded with 10 μg or 50 μg VEGF-C. 30% of the total VEGF-C is released from the gels and given the controlled release profile, 0.25% laponite alginate cryogel was chosen for in vivo work. FIG. 21D is an image depicting cryogel injected subcutaneously in mice with a 16G needle.

FIGS. 22A-22C depict induction of in vivo lymphoangiogensis by VEGF-C in naive mouse skin. Not to be bound by theory, the minor mature covalent dimeric VEGF-C appears to have superior efficacy. FIG. 22A depicts the in vivo experimental setup and FIG. 22B shows representative dot plots showing in vivo lymphangiogenesis assessed by staining of lymphatic endothelial cells (CD45−, CD31+, PDPN+) by flow cytometry after skin digestion performed 14 days after cryogel implantation. In FIG. 22C, lymphangiogenesis in mouse skin quantified after staining as lymphatic endothelial cells counts/mg. Covalent dimers (#8, #9) show high in vivo lymphangiogenesis. Accordingly, #8 was used in further experiments. PDPN: podoplanin.

FIGS. 23A-23E show that skin lymphatics respond to 10 g VEGF-C(#7 variant, SEQ ID NO: 734) delivered by alginate cryogels and lymphangiogenesis peaks 14 days after cryogel implant, which corresponds to peak immune-infiltration in naïve skin of C57/BL6 mice. FIG. 23A depicts the in vivo dose response of VEGF-C loaded into alginate cryogels (1, 10, 20, 50 μg) and lymphangiogenesis induction (represented as total lymphatic endothelial cells (LECs) counts/mg tissue, upper graph) and time course of lymphangiogenesis after delivery of 10 μg VEGF-C(lower graph). These results indicated that 10 μg of VEGF-C induced high in vivo lympahangiogenesis and that peak lymphangiogenesis was observed 14 days after subcutaneous cryogel delivery. FIG. 23B Representative plots of LECs (CD45−CD31+PDPN+) and blood endothelial cells (BECs, CD45−CD31+PDPN−) staining isolated after skin digestion of C57/BL6 mice 14 days after gel implant. FIG. 23C Quantification of endothelial cells as total cell counts/mg tissue. FIG. 23D Quantification represented as total cell counts/mg tissue of CD4+ T cells and CD8+ T cells. LECs counts correlate with T cells infiltrates, especially of a naïve phenotype (CD62L+, CD44−). Bar graphs include 5 independent experiment pooled for VEGF-C and control conditions, VEGF-C n=30, control n=18, naïve n=3 and are represented as mean+SEM, Mann-Whitney non-parametric t-test, ****, p<0.0001; **, p<0.01. FIG. 23E shows quantification of indicated cell types (LECs, CD4 T cells, or CD8 T cells) per mg of tissue over the days following VEGF-C alginate cryogel injection.

FIGS. 24A-24C confirm that VEGF-C #8 produced in CHO MaKo cells is functional. In FIG. 24A, HDLECs in vitro sprouting assay shows comparable activity of VEGF-C protein #8 produced in CHO cells (8CHO) compared to #8 produced in HEK293T cells. In FIG. 24B, comparable bioactivity was confirmed also in vivo by staining LECs after skin digestion 14 days after cryogel delivery. FIG. 24C shows quantification of lymphangiogenesis as total LECs counts/mg tissue in mice injected with blank cryogel or cryogel loaded with #8HEK or #8CHO VEGF-C. BECs (CD45−CD31+PDPN−) are not affected by the VEGF-C #8 delivery, while peak lymphangiogenesis corresponded to peak immune infiltration of CD4 and CD8 T cells (CD45+) in the skin on top of the cryogels on day 14 after cryogel delivery. n>9. Bar graphs represented as mean+SEM, Mann-Whitney non-parametric t-test between CHO vs control, HEK vs control or CHO vs HEK, ****, p<0.0001; **, p<0.01 FIGS. 25A-25C demonstrate that VEGF-C also induces lymphangiogensis in immunocompromised NSG mice and mouse LECs efficiently recruit human peripheral blood monoculear cells (PBMCs). FIG. 25A provides representative flow cytometry plots of skin LECs and BECs statining in NSG, C57/BL6 mice 14 days after VEGF-C(variant #8, minor mature form with C137A mutation, SEQ ID NO: 736) or blank cryogel delivery. FIG. 25B lays out the experimental setup of gel delivery in NSG mice and subsequent intravenous injection of human PBMCs (on day 10). Mice were euthanized and analyzed for lymphangiogenesis and immune infiltration in the skin on day 17 post gel implant. FIG. 25C shows quantification represented as total counts/mg of LECs, total T cells (CD45+CD3+) CD8 (CD3+CD8+) and CD4 (CD3+CD4+) T cells subsets, as well as B cells (CD45+CD19+). These data demonstrate that T cells are the main cell types of PBMCs recruited in NSG mice.

FIGS. 26A-26B provide a schematic of VEGF-C delivery in mouse skin generating a secondary priming site for T cells to be educated and transduced after injection of viral particles bound to mesoporous silica particles (MSPs), e.g., homogenized mesoporous silica rods (MSRs), in combination with MSP-bound STARTERS (e.g., homogenized MSR-bound STARTERS) (FIG. 26A) or free virus in combination with MSP-bound STARTERS (e.g., homogenized MSR-bound STARTERS) (FIG. 26B).

FIGS. 27A-27B provide characterization of loading capacity of MSPs, in particular homogenized MSRs, with CD 19 CAR encoding lentivirus. Trimethylammonium MSRs were co-incubated with GFP-expressing lentivirus at various amounts according to functional titer as determined by cell-based transduction assays. The amount of virus in three fractions was characterized—viral loading solution (initial input added to the MSRs), MSR-bound virus (amount remaining bound to the MSRs after incubation and washing), and unbound virus (the amount in the solution remaining after co-incubation of MSRs and virus). MSR and virus were co-incubated for 30 minutes on ice, the supernatant was removed (Unbound Virus) and the MSRs were washed twice prior to assessing MSR-bound virus. The untouched Viral Loading Solutions from each condition were also analyzed. In FIG. 27A, results show the majority of the virus in the input Virus Loading Solution is retained in the MSR-bound virus fraction after adsorption and washing. The amount of virus adsorbed to the MSRs increases with the amount of virus in the Virus Loading Solution. FIG. 27A shows the calculated fraction of MSR-bound virus relative to the Virus Loading Solution has a strong efficiency of loading and retention on the MSRs following adsorption and washing.

FIG. 28 provides characterization of retention of virus on MSPs, in particular homogenized MSRs. Lentivirus and MSRs were co-incubated for 30 minutes on ice, and the MSRs were washed twice. MSRs were then cultured in R10 medium containing 10% FCS or OpTmizer serum-free medium, and the input virus stock was incubated in media as well. The supernatant was removed at the indicated times after the start of incubation and analyzed for total virus content. Results indicate that the MSRs release only a fraction of the input virus over the first 18 hours.

FIGS. 29A-29C show functional generation of CAR-T cells using MSP, in particular homogenized MSR, bound to a CD19 CAR encoding (CAR19) virus. CARTs were generated either with free lentivirus (CAR-free) or with lentivirus bound to trimethylammonium MSRs (CAR-MSR). The MOI used for CAR-free transduction and the MOI of total virus used for adsorption to MSRs in the CAR-MSR condition is indicated. These MOIs were chosen to produce CARTs with similar transduction efficiency between the two conditions. MSRs were washed after the adsorption step, thus the total amount of virus in the transduction of the T cells in the MSR condition may occur at a lower MOI than indicated. 4.3e6 TU/virus per 1 mg of MSRs was used in the co-incubation of MSR and virus prior to washing and plating with T cells. T cells were treated with Construct 4 (Table 20, FIG. 38A-38B) and the indicated virus preparation for 1 day, followed by washing and culture for an additional 3 days. FIG. 29A shows CAR+% measured at day 4 after transduction. Cells were used in killing assays with Nalm6-Luc cells at day 4 post-transduction and co-incubated at the indicated Effector:Target (E:T ratio; T cells) after normalizing for total cells and CAR+ cells. Results indicate that FIG. 29B specific killing activity and FIG. 29C interferon-gamma release during the co-incubation for 24 hours were comparable between CAR-MSR and CAR-free, showing that transduction by formulation with MSRs produces equivalently functional CARTs in vitro compared to conventional free virus transduction.

FIGS. 30A-30B demonstrate that MSR injectability was improved through size reduction by homogenization. MSRs were homogenized using a bead mixer to reduce their size and improve injectability. In FIG. 30A, size reduction was seen in terms of MSR length, and this allowed injection through a smaller diameter needle into the intradermal space. In FIG. 30A, standard trimethylammonium MSRs or homogenized MSRs were adsorbed with lentivirus and a dilution series of this complex was created and used to transduce T cells with a GFP-encoding lentivirus. Homogenization of MSRs did not substantially alter transduction performance in vitro.

FIGS. 31A-31B depict an hematoxylin and eosin (H&E) stained section of skin containing adjacent cryogel and MSPs, in particular homogenized MSRs. Blank alginate cryogels were injected subcutaneously and 7 days later viral particles (4e6 TU) free or bound to MSRs were injected in the intradermal space on top of the gel with insulin syringe (for MSR-virus group) or Hamilton syringe (for free virus). 72h after viral delivery, mice were euthanized, and tissues (skin and draining lymph node) were harvested for immunohistochemistry analysis. To facilitate the delivery of VEGF-C to the lymphatic capillaries, the cryogel was implanted into the high subcutaneous space on top of the panniculus muscle. FIG. 31A, which depicts H&E stained sections, demonstrated the location of the subcutaneous cryogel superficial to the panniculus muscle in the hypodermis. The MSPs, in particular homogenized MSRs, appeared as lightly eosinophilic granular material admixed with mononuclear cells positioned at the dermal-hypodermal junction adjacent to the implanted cryogel (FIG. 31A and FIG. 31B for close up).

FIGS. 32A-32B show in situ hybridization for CAR mRNA on sections of skin. In situ hybridization to detect CAR mRNA transcript demonstrated robust signal within regions corresponding to the injected MSP, in particular homogenized MSR, in mice injected with MSR-bound virus (FIG. 32A), while it detected diffuse signal in cells infiltrating the gel, as well as cells adjacent to it in the free virus condition (FIG. 32B). Not to be bound by theory, Applicants believe these data support the notion that MSPs, in particular homogenized MSRs, may maintain the virus localized in the dermis where the T cells are infiltrating the skin.

FIGS. 33A-33B show a mouse injected with MSP-virus, in particular homogenized MSR-virus, had less CAR mRNA transcript positive cells in draining lymph node compared to free virus group. In situ hybridization for CAR mRNA on sections of draining lymph node (dLN). In situ hybridization detected only one CAR mRNA transcript positive cells within the dLN of mice injected with MSR-bound virus (FIG. 33A), while mice injected with free-virus showed few CAR mRNA transcript positive cells in the subcapsular sinus consistent with local drainage of the virus or the cells from the site of cryogel implantation (FIG. 33B).

FIGS. 34A-34C show the generation of CD19+ CART cells in vivo and B cells depletion in the spleen. FIG. 34A provides a timeline for experimental setup of in vivo CART manufacturing. On day 0 VEGF-C loaded alginate cryogels were injected subcutaneously in NSG mice. On day 10 (3 days before we know peak lymphangiogeneiss is achieved), PBMCs are intravenously injected into mice and 7 days later (on day 17) MSP-virus (in particular homogenized MSR-virus), free-virus or PBS as control were injected in the respective groups together with MSP-STARTERS, in particular homogenized MSR-STARTERS, to possibly promote T cells activation and favor T cells transduction. On day 35 mice were euthanized and spleen, blood and can were collected to determine if in vivo CD19 CAR encoding viral delivery elicited transduction of T Cells recruited by VEGF-C induced lymphangiogenesis. FIG. 34B provides representative flow cytometry (FACS) plots of immune populations showing B cells depletion and T cells transduction (CD19 CAR+ cells) in the spleen of mice treated with free virus or MSP-virus, in particular homogenized MSR-virus. FIG. 34C is a quantification of B cells depletion and CAR-T cells in the spleen of the mice in all groups.

FIG. 35 demonstrates that minimal transduction of non-T cells by the composition. Provided are representative flow cytometry (FACS) plots of human CD11b+ monocytes, mouse monocytes (non-functional in NSG mouse), as well as stromal cells in the spleen of mice treated with free virus or MSP-virus, in particular homogenized MSR-virus, or PBS control. Few human monocytic cells showed a positive staining for CD 19 CAR.

FIGS. 36A-36B show B cell depletion correlates with CAR-T cell expansion in spleen and blood. In FIG. 36A, CART cells (total counts/mg tissue) are plotted versus B cells (total counts/mg tissue) in spleen, and, in FIG. 36B, CART cells (blood, represented as total cell counts/μl) are plotted versus B cell depletion (spleen, represented as total cell counts/mg tissue).

FIG. 37A-37C provide exemplary schema for bispecific antibodies, including single bispecific antibody schema (FIG. 37A), multimeric bispecific antibody schema (FIG. 37B), and a figure legend (FIG. 37C).

FIG. 38. T cells are recruited at the site of implant. FIG. 38 shows experimental setup (above) and representative image (below) of IHC analysis of CD3 on mouse skin receiving the cryogel-VEGF-C implant plus MSP-virus and MSP-STARTERS 18 days after viral delivery.

FIG. 39. CAR ISH signal in mononuclear cells surrounding the gel. FIG. 39 shows representative images of CAR ISH analysis (CD19 CAR RNA) of cells in mouse skin receiving the cryogel-VEGF-C implant plus MSP-virus and MSP-STARTERS 18 days after viral delivery.

FIG. 40. Locally in vivo generated CAR-T cells migrate to the spleen and correlate with B cell depletion. FIG. 40 is representative image of CAR ISH analysis of cells in mouse spleen receiving the cryogel-VEGF-C implant plus MSP-virus and MSP-STARTERS 18 days after viral delivery.

FIGS. 41A-41B. Generation of in vivo CAR-T cells correlates with B cells killing at the implant site and in the spleen. Representative image of fluorescently labelled CD19+B cells and CD3+ T cells at the implant site (FIG. 41A, fluorescent image upper panel and brightfield image lower panel), as well as in the spleen (FIG. 41B) of mice receiving the cryogel-VEGF-C implant plus MSP-virus and MSP-STARTERS 18 days after viral delivery.

FIGS. 42A-42B. Analysis of circulating human T cells in NSG mice over time. FIG. 42A shows experimental design and groups. FIG. 42B is a set of graphs showing flow cytometry analysis over time of implanted mice. Flow cytometry data represented as Mean±SEM.

FIGS. 43A-43B. B cell depletion in circulation correlates with CART cells expansion. Flow cytometry analysis over time of CART cells (FIG. 43A) and B cells (FIG. 43B) in the circulation of implanted mice. Flow cytometry data represented as Mean±SEM (in FIGS. 43A and 43B) or as individual curves for each mouse (FIG. 43B).

FIGS. 44A-44B. Strong correlation of CART cell expansion with B cell depletion in the circulation and in the spleen. Correlation of B cell number and T cell number in the blood (FIG. 44A) and in the spleen (FIG. 44B) of mice treated with the different conditions. Cell number represents cell counts/mg tissue or counts/μl for blood determined during flow cytometry analysis.

FIGS. 45A-45B. Quantification of CART cell expansion and corresponding B cell depletion in the spleen 18 days after viral delivery. Quantification of CART (FIG. 45A) and B cells (FIG. 45B) counts/mg tissue in the spleen of treated mice. Flow cytometry data represented as Mean±SEM.

FIGS. 46A-46D. CART cell expansion correlates to lymphangiogenesis as well as local B cell depletion in the skin 18 days after viral delivery. Quantification of CART (FIG. 46A) and B cells (FIG. 46B) counts/mg tissue in the spleen of treated mice. Flow cytometry data represented as Mean±SEM. Correlation plot of B cell number and CART cell number (FIG. 46C) as well as CART cell number and lymphatic endothelial cell (LEC) number (FIG. 46D) in the skin. In correlation plots, cell numbers are represented as counts/mg tissue.

FIG. 47. GFP transgene expression as a function of MSP dose.

FIG. 48A-48B depicts schema of the 17 different multispecific constructs comprising a CD3 antigen binding domain comprising a heavy and light chain derived from an anti-CD3 antibody and, in all but control Constructs 11, 14, and 17, an a CD28 or CD2 antigen binding domain, as noted, comprising a heavy and light chain derived from an anti-CD28 or CD2 antibody, respectively. Not to be bound by theory, it is appreciated that any one or more of these constructs may be used as a cell activation agent as disclosed herein.

Construct 1 comprises an anti-CD3 scFv fused to an anti-CD2 Fab, which is further fused to an Fc region. Construct 1 comprises a first chain and a second chain. The first chain comprises, from the N-terminus to the C-terminus, anti-CD2 VL and CL. The second chain comprises, from the N-terminus to the C-terminus, anti-CD3 VH, (G4S)4 linker (SEQ ID NO: 29), anti-CD3 VL, (G4S)4 linker (SEQ ID NO: 29), anti-CD2 VH, CH1, CH2, and CH3. Construct 2 comprises an anti-CD3 scFv fused to an anti-CD28 Fab, which is further fused to an Fc region. Construct 2 comprises a first chain and a second chain. The first chain comprises, from the N-terminus to the C-terminus, anti-CD28 VL and CL. The second chain comprises, from the N-terminus to the C-terminus, anti-CD3 VH, (G4S)4 linker (SEQ ID NO: 29), anti-CD3 VL, (G4S)4 linker (SEQ ID NO: 29), anti-CD28 VH, CH1, CH2, and CH3. Construct 3 comprises an anti-CD2 Fab fused to an Fc region, which is further fused to an anti-CD3 scFv. Construct 3 comprises a first chain and a second chain. The first chain comprises, from the N-terminus to the C-terminus, anti-CD2 VL and CL. The second chain comprises, from the N-terminus to the C-terminus, anti-CD2 VH, CH1, CH2, CH3, (G4S)4 linker (SEQ ID NO: 29), anti-CD3 VH, (G4S)4 linker (SEQ ID NO: 29), anti-CD3 VL. Construct 4 comprises an anti-CD28 Fab fused to an Fc region, which is further fused to an anti-CD3 scFv. Construct 4 comprises a first chain and a second chain. The first chain comprises, from the N-terminus to the C-terminus, anti-CD28 VL and CL. The second chain comprises, from the N-terminus to the C-terminus, anti-CD28 VH, CH1, CH2, CH3, (G4S)4 linker (SEQ ID NO: 29), anti-CD3 VH, (G4S)4 linker (SEQ ID NO: 29), anti-CD3 VL. Construct 5 comprises an anti-CD2 Fab fused to an anti-CD3 scFv, which is further fused to an Fc region. Construct 5 comprises a first chain and a second chain. The first chain comprises, from the N-terminus to the C-terminus, anti-CD2 VL and CL. The second chain comprises, from the N-terminus to the C-terminus, anti-CD2 VH, CH1, (G4S)2 linker (SEQ ID NO: 767), anti-CD3 VH, (G4S)4 linker (SEQ ID NO: 29), anti-CD3 VL, (G4S)4 linker (SEQ ID NO: 29), CH2, and CH3. Construct 6 comprises an anti-CD28 Fab fused to an anti-CD3 scFv, which is further fused to an Fc region. Construct 6 comprises a first chain and a second chain. The first chain comprises, from the N-terminus to the C-terminus, anti-CD28 VL and CL. The second chain comprises, from the N-terminus to the C-terminus, anti-CD28 VH, CH1, (G4S)2 linker (SEQ ID NO: 767), anti-CD3 VH, (G4S)4 linker (SEQ ID NO: 29), anti-CD3 VL, (G4S)4 linker (SEQ ID NO: 29), CH2, and CH3.

Construct 7 comprises an anti-CD3 scFv fused to an Fc region, which is further fused to an anti-CD2 Fab. Construct 7 comprises a first chain and a second chain. The first chain comprises, from the N-terminus to the C-terminus, anti-CD2 VL and CL. The second chain comprises, from the N-terminus to the C-terminus, anti-CD3 VH, (G4S)4 linker (SEQ ID NO: 29), anti-CD3 VL, (G4S) linker (SEQ ID NO: 768), CH2, CH3, (G4S)4 linker (SEQ ID NO: 29), anti-CD2 VH, and CH1. Construct 8 comprises an anti-CD3 scFv fused to an Fc region, which is further fused to an anti-CD28 Fab. Construct 8 comprises a first chain and a second chain. The first chain comprises, from the N-terminus to the C-terminus, anti-CD28 VL and CL. The second chain comprises, from the N-terminus to the C-terminus, anti-CD3 VH, (G4S)4 linker (SEQ ID NO: 29), anti-CD3 VL, (G4S) linker (SEQ ID NO: 768), CH2, CH3, (G4S)4 linker (SEQ ID NO: 29), anti-CD28 VH, and CH1.

Construct 9 comprises an anti-CD2 Fab fused to a first Fc region and an anti-CD3 scFv fused to a second Fc region. Construct 9 comprises a first chain, a second chain, and a third chain. The first chain comprises, from the N-terminus to the C-terminus, anti-CD2 VL and CL. The second chain comprises, from the N-terminus to the C-terminus, anti-CD2 VH, CH1, CH2, and CH3. The third chain comprises, from the N-terminus to the C-terminus, anti-CD3 VH, (G4S)4 linker (SEQ ID NO: 29), anti-CD3 VL, (G4S) linker (SEQ ID NO: 768), CH2, and CH3. Construct 10 comprises an anti-CD28 Fab fused to a first Fc region and an anti-CD3 scFv fused to a second Fc region. Construct 10 comprises a first chain, a second chain, and a third chain. The first chain comprises, from the N-terminus to the C-terminus, anti-CD28 VL and CL. The second chain comprises, from the N-terminus to the C-terminus, anti-CD28 VH, CH1, CH2, and CH3. The third chain comprises, from the N-terminus to the C-terminus, anti-CD3 VH, (G4S)4 linker (SEQ ID NO: 29), anti-CD3 VL, (G4S) linker (SEQ ID NO: 768), CH2, and CH3. Construct 11 comprises an anti-CD3 scFv fused to an Fc region. Construct 11 comprises a first chain and a second chain. The first chain comprises, from the N-terminus to the C-terminus, CH2 and CH3. The second chain comprises, from the N-terminus to the C-terminus, anti-CD3 VH, (G4S)4 linker (SEQ ID NO: 29), anti-CD3 VL, (G4S) linker (SEQ ID NO: 768), CH2, and CH3.

Construct 12 comprises an anti-CD2 Fab fused to a first Fc region and an anti-CD3 scFv fused to a second Fc region. Construct 12 comprises a first chain, a second chain, and a third chain. The first chain comprises, from the N-terminus to the C-terminus, anti-CD2 VL and CL. The second chain comprises, from the N-terminus to the C-terminus, anti-CD2 VH, CH1, CH2, and CH3. The third chain comprises, from the N-terminus to the C-terminus, anti-CD3 VH, (G4S)4 linker (SEQ ID NO: 29), anti-CD3 VL, (G4S) linker (SEQ ID NO: 768), CH2, CH3, (G4S)3 linker (SEQ ID NO: 30), and Matrilin1. Construct 13 comprises an anti-CD28 Fab fused to a first Fc region and an anti-CD3 scFv fused to a second Fc region. Construct 13 comprises a first chain, a second chain, and a third chain. The first chain comprises, from the N-terminus to the C-terminus, anti-CD28 VL and CL. The second chain comprises, from the N-terminus to the C-terminus, anti-CD28 VH, CH1, CH2, and CH3. The third chain comprises, from the N-terminus to the C-terminus, anti-CD3 VH, (G4S)4 linker (SEQ ID NO: 29), anti-CD3 VL, (G4S) linker (SEQ ID NO: 768), CH2, CH3, (G4S)3 linker (SEQ ID NO: 30), and Matrilin1. Construct 14 comprises an anti-CD3 scFv fused to an Fc region. Construct 14 comprises a first chain and a second chain. The first chain comprises, from the N-terminus to the C-terminus, CH2 and CH3. The second chain comprises, from the N-terminus to the C-terminus, anti-CD3 VH, (G4S)4, linker (SEQ ID NO: 29), anti-CD3 VL, (G4S) linker (SEQ ID NO: 768), CH2, CH3, (G4S)3 linker (SEQ ID NO: 30), and Matrilin1.

Construct 15 comprises an anti-CD2 Fab fused to a first Fc region and an anti-CD3 scFv fused to a second Fc region. Construct 15 comprises a first chain, a second chain, and a third chain. The first chain comprises, from the N-terminus to the C-terminus, anti-CD2 VL and CL. The second chain comprises, from the N-terminus to the C-terminus, anti-CD2 VH, CH1, CH2, and CH3. The third chain comprises, from the N-terminus to the C-terminus, anti-CD3 VH, (G4S)4 linker (SEQ ID NO: 29), anti-CD3 VL, (G4S) linker (SEQ ID NO: 768), CH2, CH3, (G4S) linker (SEQ ID NO: 768), and the coiled-coil domain of cartilage oligomeric matrix protein (COMPcc). Construct 16 comprises an anti-CD28 Fab fused to a first Fc region and an anti-CD3 scFv fused to a second Fc region. Construct 16 comprises a first chain, a second chain, and a third chain. The first chain comprises, from the N-terminus to the C-terminus, anti-CD28 VL and CL. The second chain comprises, from the N-terminus to the C-terminus, anti-CD28 VH, CH1, CH2, and CH3. The third chain comprises, from the N-terminus to the C-terminus, anti-CD3 VH, (G4S)4 linker (SEQ ID NO: 29), anti-CD3 VL, (G4S) linker (SEQ ID NO: 768), CH2, CH3, (G4S) linker (SEQ ID NO: 768), and COMPcc.

Construct 17 comprises an anti-CD3 scFv fused to an Fc region. Construct 17 comprises a first chain and a second chain. The first chain comprises, from the N-terminus to the C-terminus, CH2 and CH3. The second chain comprises, from the N-terminus to the C-terminus, anti-CD3 VH, (G4S)4 linker (SEQ ID NO: 29), anti-CD3 VL, (G4S) linker (SEQ ID NO: 768), CH2, CH3, (G4S) linker (SEQ ID NO: 768), and COMPcc.

Exemplary sequences of Construct 1 to Construct 17 are provided in Table 20. Additional sequences (e.g., the anti-CD3 binders disclosed herein, the anti-CD28 binders disclosed herein, the anti-CD2 binders disclosed herein, or the Fc regions disclosed herein) can be used to generate Construct 1 to Construct 17.

FIGS. 49A-49B shows the binding information (FIG. 49A) and configuration (FIG. 49B) of second generation STARTERS molecules. “F5 ANTI-CD3 (2)” refers to an F5 construct with an anti-CD3 binder based on ANTI-CD3 (2).

FIGS. 50A-50B show the binder information (FIG. 50A) and configuration (FIG. 50B) of third generation STARTERS molecules.

FIG. 51A shows the configuration of the STARTERS molecule tested in Example J. FIGS. 51B and 51C show T cell activation and transduction mediated by MSP-lentivirus-STARTERS mixtures. The concentration of the STARTERS molecule in each dilution of the MSP-lentivirus-STARTERS mixture is shown on the x-axis. The formulations generated from both MSP batches yielded similar T cell activation (FIG. 51B) and transduction (FIG. 51C) efficiencies in vitro. Delivering the STARTERS molecule bound to MSPs (“Batch 1” and “Batch 2” in FIGS. 51B-C) enhanced activation and transduction as compared to soluble delivery of the STARTERS molecule (“No MSP” in FIGS. 51B-C). Day 1 in FIG. 51B and Day 4 in FIG. 51C refer to the time after the start of the T cell culture (i.e., when the cells and MSP-STARTERS-virus were initially mixed together).

FIGS. 52A and 52B show T cell activation and transduction mediated by MSP-lentivirus-STARTERS mixtures. GFP-encoding lentivirus and the STARTERS molecule were loaded onto full-size (“MSP”) or size-reduced MSPs, where size reduction was achieved using bead homogenization (“Bead homogenized”) or sonication (“Sonicated”) of MSPs. The ratio of lentivirus to T cells (MOI) in each dilution of the MSP-lentivirus-STARTERS mixture is shown on the x-axis. Size reduction of MSPs did not negatively impact in vitro potency with respect to T cell activation (FIG. 52A) and transduction (FIG. 52B). Both bead homogenization and sonication yielded comparable results to the full-sized MSPs.

FIGS. 53A and 53B show the design of the in vivo study. FIG. 53C is a correlation plot between CD19 CAR-T expansion and B cell depletion in the blood of mice at day 18 post-injectable 2 injection. FIG. 53D shows bioluminescence measurements of luciferase-expressing NALM6 tumors engrafted into NSG mice for 4 days, and then treated with a dose of 3e5 adoptively transferred CAR+ T cells from an initial cohort of mice undergoing the in vivo CART generation process. Images are from 13 days post-adoptive transfer. Mice were treated with a dose of 3e5 CAR-Ts manufactured using free virus (“Free virus”) or MSP-delivered virus (“MSP virus”). Also shown are control groups where tumors were treated with transferred PBMCs from donor mice with no CARTs (“PBMC control”) or where tumors were not untreated (“NALM6”). The mouse labeled “a” received 1.5e5 CAR+ cells from the free virus group. The mouse labeled “b” received 2.3e5 CAR+ cells from the free virus group. FIGS. 53E, 53F, and 53G are plots showing CART % of total T cells in circulation (FIG. 53E), the amount of CARTs in circulation (FIG. 53F), and the amount of circulating CD3+ T cells (FIG. 53G) 13 days after intradermal injection of injectable 2 at the cryogel site. FIGS. 53H, 53I, and 53J are plots of mice selected for circulating lymphocyte adoptive transfer into NALM6 challenged mice, showing the amount of circulating T cells (FIG. 53H), the amount of circulating CART cells (FIG. 53I), and CART % of total T cells in circulation (FIG. 53J) 18 days after intradermal injection of injectable 2. FIGS. 53K and 53L show results from combined flow cytometry analysis of blood from mice used for adoptive transfer and remaining mice enrolled in the study, showing the amount of circulating T cells (FIG. 53K) and the amount of CART cells in circulation (FIG. 53L) 18 or 19 days after intradermal injection of injectable 2. FIGS. 54A, 54B, 54C, and 54D are graphs showing in vitro release data. FIG. 54A shows data for H2a hydogel (200 kDa [HA-N₃]-24%; 9% crosslinked) and H4a hydrogel (700 kDa [HA-N₃]-16%; 18% crosslinked). FIG. 54B shows data for H4a hydrogel (700 kDa [HA-N₃]-16%; 18% crosslinked; no laponite), H5a hydrogel (700 kDa [HA-N₃]-16%; 18% crosslinked; 0.25 mg/ml laponite), and H6a hydrogel (700 kDa [HA-N₃]-16%; 18% crosslinked; 1 mg/ml laponite). FIG. 54C shows data for H5a hydrogel (in situ; 0.25 mg/ml laponite) and H5b hydrogel (particles; 0.25 mg/ml laponite). FIG. 54D shows data for H6a hydrogel (in situ; 1 mg/ml laponite) and H6b hydrogel (particles; 1 mg/ml laponite).

FIGS. 55A and 55B are graphs showing in vivo PD response at day 7.

### DETAILED DESCRIPTION

### Definitions

Unless defined otherwise, all technical and scientific terms used herein have the same meaning as commonly understood by one of ordinary skill in the art to which the invention pertains.

The term “a” and “an” refers to one or to more than one (i.e., to at least one) of the grammatical object of the article. By way of example, “an element” means one element or more than one element.

The term “about” when referring to a measurable value such as an amount, a temporal duration, and the like, is meant to encompass variations of 20% or in some instances ±10%, or in some instances ±5%, or in some instances+1%, or in some instances+0.1% from the specified value, as such variations are appropriate to perform the disclosed methods.

As used herein, the term “cell recruitment factor” refers to an agent capable of recruiting cells, for example immune cells. Non-limiting examples of such recruitment factors include IL-2, IL-7, CCL21, IL 5, GM-CSF, CCL19, CXCL9, CXCL10, XCL1, lymphotoxin alpha, lymphotoxin beta, and VEGF-C. Such factors are known to recruit immune cells, such as T-cells. Further non-limiting examples of cell recruitment factors that recruit specific cell types include skin homing chemokines such as but not limited to CCL17, CCL22, CCL20, and CCL27; myeloid cell chemoattractants such as but not limited to FLT3L, G-CSF, PDGF, S100A8/A9, CSF-1, CXCL8, CCL20, CCL17, CCR5, CCR6, CCL2, VEGF, Angiopoietin-2, CXCL12, PGE2, and LTB4; and NK-specific recruitment factors such as but not limited to CXC3L1, CCL19, CCL21, CXCL10, CXCL11, and CXCL12

As used herein, the term “cell activation agent” refers to an agent capable of activating a cell to perform a given function—for example, with T-cells, engagement of endogenous or engineered receptors (e.g., a CAR) or cell-surface markers activate T-cells to proliferate and, in certain cases, secrete appropriate cytokines.

The term “Chimeric Antigen Receptor” or alternatively a “CAR” refers to a recombinant polypeptide construct comprising at least an extracellular antigen binding domain, a transmembrane domain and a cytoplasmic signaling domain (also referred to herein as “an intracellular signaling domain”) comprising a functional signaling domain derived from a stimulatory molecule as defined below. In some embodiments, the domains in the CAR polypeptide construct are in the same polypeptide chain, e.g., comprise a chimeric fusion protein. In some embodiments, the domains in the CAR polypeptide construct are not contiguous with each other, e.g., are in different polypeptide chains, e.g., as provided in an RCAR as described herein.

In some aspects, the cytoplasmic signaling domain comprises a primary signaling domain (e.g., a primary signaling domain of CD3-zeta). In some aspects, the cytoplasmic signaling domain further comprises one or more functional signaling domains derived from at least one costimulatory molecule as defined below. In some aspects, the costimulatory molecule is chosen from 41BB (i.e., CD137), CD27, ICOS, and/or CD28. In some aspects, the CAR comprises a chimeric fusion protein comprising an extracellular antigen recognition domain, a transmembrane domain and an intracellular signaling domain comprising a functional signaling domain derived from a stimulatory molecule. In some aspects, the CAR comprises a chimeric fusion protein comprising an extracellular antigen recognition domain, a transmembrane domain and an intracellular signaling domain comprising a functional signaling domain derived from a co-stimulatory molecule and a functional signaling domain derived from a stimulatory molecule. In some aspects, the CAR comprises a chimeric fusion protein comprising an extracellular antigen recognition domain, a transmembrane domain and an intracellular signaling domain comprising two functional signaling domains derived from one or more co-stimulatory molecule(s) and a functional signaling domain derived from a stimulatory molecule. In some aspects, the CAR comprises a chimeric fusion protein comprising an extracellular antigen recognition domain, a transmembrane domain and an intracellular signaling domain comprising at least two functional signaling domains derived from one or more co-stimulatory molecule(s) and a functional signaling domain derived from a stimulatory molecule. In some aspects the CAR comprises an optional leader sequence at the amino-terminus (N-ter) of the CAR fusion protein. In some aspects, the CAR further comprises a leader sequence at the N-terminus of the extracellular antigen recognition domain, wherein the leader sequence is optionally cleaved from the antigen recognition domain (e.g., an scFv) during cellular processing and localization of the CAR to the cellular membrane.

A CAR that comprises an antigen binding domain (e.g., an scFv, a single domain antibody, or TCR (e.g., a TCR alpha binding domain or TCR beta binding domain)) that targets a specific tumor marker X, wherein X can be a tumor marker as described herein, is also referred to as XCAR. For example, a CAR that comprises an antigen binding domain that targets BCMA is referred to as BCMA CAR. The CAR can be expressed in any cell, e.g., an immune effector cell as described herein (e.g., a T cell or an NK cell).

The term “signaling domain” refers to the functional portion of a protein which acts by transmitting information within the cell to regulate cellular activity via defined signaling pathways by generating second messengers or functioning as effectors by responding to such messengers.

The term “antibody,” as used herein, refers to a protein, or polypeptide sequence derived from an immunoglobulin molecule which specifically binds with an antigen. Antibodies can be polyclonal or monoclonal, multiple or single chain, or intact immunoglobulins, and may be derived from natural sources or from recombinant sources. Antibodies can be tetramers of immunoglobulin molecules.

The term “antibody fragment” refers to at least one portion of an antibody, that retains the ability to specifically interact with (e.g., by binding, steric hindrance, stabilizing/destabilizing, spatial distribution) an epitope of an antigen. Examples of antibody fragments include, but are not limited to, Fab, Fab □F(ab)2, Fv fragments, scFv antibody fragments, disulfide-linked Fvs (sdFv), a Fd fragment consisting of the VH and CH1 domains, linear antibodies, single domain antibodies such as sdAb (either VL or VH), camelid VHH domains, multi-specific antibodies formed from antibody fragments such as a bivalent fragment comprising two Fab fragments linked by a disulfide bridge at the hinge region, and an isolated CDR or other epitope binding fragments of an antibody. An antigen binding fragment can also be incorporated into single domain antibodies, maxibodies, minibodies, nanobodies, intrabodies, diabodies, triabodies, tetrabodies, v-NAR and bis-scFv (see, e.g., Hollinger and Hudson, Nature Biotechnology 23:1126-1136, 2005). Antigen binding fragments can also be grafted into scaffolds based on polypeptides such as a fibronectin type III (Fn3) (see U.S. Pat. No. 6,703,199, which describes fibronectin polypeptide minibodies).

The portion of the CAR of the invention comprising an antibody or antibody fragment thereof may exist in a variety of forms where the antigen binding domain is expressed as part of a contiguous polypeptide chain including, for example, a single domain antibody fragment (sdAb), a single chain antibody (scFv), a humanized antibody or bispecific antibody (Harlow et al., 1999, In: Using Antibodies: A Laboratory Manual, Cold Spring Harbor Laboratory Press, NY; Harlow et al., 1989, In: Antibodies: A Laboratory Manual, Cold Spring Harbor, New York; Houston et al., 1988, Proc. Natl. Acad. Sci. USA 85:5879-5883; Bird et al., 1988, Science 242:423-426). In some aspects, the antigen binding domain of a CAR composition of the invention comprises an antibody fragment. In a further aspect, the CAR comprises an antibody fragment that comprises a scFv. The precise amino acid sequence boundaries of a given CDR can be determined using any of a number of well-known schemes, including those described by Kabat et al. (1991), “Sequences of Proteins of Immunological Interest,” 5th Ed. Public Health Service, National Institutes of Health, Bethesda, MD (“Kabat” numbering scheme), A1-Lazikani et al., (1997) JMB 273,927-948 (“Chothia” numbering scheme), or a combination thereof.

As used herein, the term “binding domain” or “antibody molecule” refers to a protein, e.g., an immunoglobulin chain or fragment thereof, comprising at least one immunoglobulin variable domain sequence. The term “binding domain” or “antibody molecule” encompasses antibodies and antibody fragments. In some embodiments, an antibody molecule is a multispecific antibody molecule, e.g., it comprises a plurality of immunoglobulin variable domain sequences, wherein a first immunoglobulin variable domain sequence of the plurality has binding specificity for a first epitope and a second immunoglobulin variable domain sequence of the plurality has binding specificity for a second epitope. In some embodiments, a multispecific antibody molecule is a bispecific antibody molecule. A bispecific antibody has specificity for no more than two antigens. A bispecific antibody molecule is characterized by a first immunoglobulin variable domain sequence which has binding specificity for a first epitope and a second immunoglobulin variable domain sequence that has binding specificity for a second epitope.

The terms “bispecific antibody” and “bispecific antibodies” refer to molecules that combine the antigen binding sites of two antibodies within a single molecule. Thus, a bispecific antibody is able to bind two different antigens simultaneously or sequentially. Methods for making bispecific antibodies are well known in the art. Various formats for combining two antibodies are also known in the art. Forms of bispecific antibodies of the invention include, but are not limited to, a diabody, a single-chain diabody, Fab dimerization (Fab-Fab), Fab-scFv, and a tandem antibody, as known to those of skill in the art.

The term “antibody heavy chain,” refers to the larger of the two types of polypeptide chains present in antibody molecules in their naturally occurring conformations, and which normally determines the class to which the antibody belongs.

The term “antibody light chain,” refers to the smaller of the two types of polypeptide chains present in antibody molecules in their naturally occurring conformations. Kappa (1) and lambda (k) light chains refer to the two major antibody light chain isotypes.

The term “recombinant antibody” refers to an antibody which is generated using recombinant DNA technology, such as, for example, an antibody expressed by a bacteriophage or yeast expression system. The term should also be construed to mean an antibody which has been generated by the synthesis of a DNA molecule encoding the antibody and which DNA molecule expresses an antibody protein, or an amino acid sequence specifying the antibody, wherein the DNA or amino acid sequence has been obtained using recombinant DNA or amino acid sequence technology which is available and well known in the art.

The term “antigen” or “Ag” refers to a molecule that provokes an immune response. This immune response may involve either antibody production, or the activation of specific immunologically competent cells, or both. The skilled artisan will understand that any macromolecule, including virtually all proteins or peptides, can serve as an antigen. Furthermore, antigens can be derived from recombinant or genomic DNA. A skilled artisan will understand that any DNA, which comprises a nucleotide sequences or a partial nucleotide sequence encoding a protein that elicits an immune response, therefore encodes an “antigen” as that term is used herein. Furthermore, one skilled in the art will understand that an antigen need not be encoded solely by a full-length nucleotide sequence of a gene. It is readily apparent that the present invention includes, but is not limited to, the use of partial nucleotide sequences of more than one gene and that these nucleotide sequences are arranged in various combinations to encode polypeptides that elicit the desired immune response. Moreover, a skilled artisan will understand that an antigen need not be encoded by a “gene” at all. It is readily apparent that an antigen can be synthesized or can be derived from a biological sample or might be a macromolecule besides a polypeptide. Such a biological sample can include, but is not limited to a tissue sample, a tumor sample, a cell or a fluid with other biological components.

The term “multispecific binding molecule” refers to a molecule that specifically binds to at least two antigens and comprise two or more antigen-binding domains. The antigen-binding domains can each independently be an antibody fragment (e.g., scFv, Fab, nanobody), a ligand, or a non-antibody derived binder (e.g., fibronectin, Fynomer, DARPin).

The term “monovalent” as used herein in the context of a multispecific binding molecule, antibody (e.g., bispecific antibody), or antibody fragment refers to a multispecific binding molecule, antibody (e.g., bispecific antibody), or antibody fragment in which there is a single antigen binding domain for each antigen to which the multispecific binding molecule, antibody (e.g., bispecific antibody), or antibody fragment binds.

The term “bivalent” as used herein in the context of a multispecific binding molecule, antibody (e.g., bispecific antibody), or antibody fragment refers to a multispecific binding molecule, antibody (e.g., bispecific antibody), or antibody fragment in which there is are two antigen binding domains for each antigen to which the multispecific binding molecule, antibody (e.g., bispecific antibody), or antibody fragment binds.

The term “multimer” refers to an aggregate of a plurality of molecules (such as but not limited to antibodies (e.g. bispecific antibodies), optionally conjugated to one another.

The term “anti-cancer effect” refers to a biological effect which can be manifested by various means, including but not limited to, e.g., a decrease in tumor volume, a decrease in the number of cancer cells, a decrease in the number of metastases, an increase in life expectancy, decrease in cancer cell proliferation, decrease in cancer cell survival, or amelioration of various physiological symptoms associated with the cancerous condition. An “anti-cancer effect” can also be manifested by the ability of the peptides, polynucleotides, cells and antibodies in prevention of the occurrence of cancer in the first place. The term “anti-tumor effect” refers to a biological effect which can be manifested by various means, including but not limited to, e.g., a decrease in tumor volume, a decrease in the number of tumor cells, a decrease in tumor cell proliferation, or a decrease in tumor cell survival.

The term “autologous” refers to any material derived from the same individual to whom it is later to be re-introduced into the individual.

The term “allogeneic” refers to any material derived from a different animal of the same species as the individual to whom the material is introduced. Two or more individuals are said to be allogeneic to one another when the genes at one or more loci are not identical. In some aspects, allogeneic material from individuals of the same species may be sufficiently unlike genetically to interact antigenically.

The term “xenogeneic” refers to a graft derived from an animal of a different species.

The term “cancer” refers to a disease characterized by the uncontrolled growth of aberrant cells. Cancer cells can spread locally or through the bloodstream and lymphatic system to other parts of the body. Examples of various cancers are described herein and include but are not limited to, breast cancer, prostate cancer, ovarian cancer, cervical cancer, skin cancer, pancreatic cancer, colorectal cancer, renal cancer, liver cancer, brain cancer, lymphoma, leukemia, lung cancer and the like. The terms “tumor” and “cancer” are used interchangeably herein, e.g., both terms encompass solid and liquid, e.g., diffuse or circulating, tumors. As used herein, the term “cancer” or “tumor” includes premalignant, as well as malignant cancers and tumors.

As described herein, “conjugated to” means associated with or attached to by any means as described herein, optionally covalently or non-covalently and/or directly or via linker.

“Derived from” as that term is used herein, indicates a relationship between a first and a second molecule. It generally refers to structural similarity between the first molecule and a second molecule and does not connotate or include a process or source limitation on a first molecule that is derived from a second molecule. For example, in the case of an intracellular signaling domain that is derived from a CD3zeta molecule, the intracellular signaling domain retains sufficient CD3zeta structure such that it has the required function, namely, the ability to generate a signal under the appropriate conditions. It does not connotate or include a limitation to a particular process of producing the intracellular signaling domain, e.g., it does not mean that, to provide the intracellular signaling domain, one must start with a CD3zeta sequence and delete unwanted sequence, or impose mutations, to arrive at the intracellular signaling domain.

The phrase “disease associated with expression of a tumor antigen as described herein” includes, but is not limited to, a disease associated with expression of a tumor antigen as described herein or condition associated with cells which express a tumor antigen as described herein including, e.g., proliferative diseases such as a cancer or malignancy or a precancerous condition such as a myelodysplasia, a myelodysplastic syndrome or a preleukemia; or a noncancer related indication associated with cells which express a tumor antigen as described herein. In some aspects, a cancer associated with expression of a tumor antigen as described herein is a hematological cancer. In some aspects, a cancer associated with expression of a tumor antigen as described herein is a solid cancer. Further diseases associated with expression of a tumor antigen described herein include, but not limited to, e.g., atypical and/or non-classical cancers, malignancies, precancerous conditions or proliferative diseases associated with expression of a tumor antigen as described herein. Non-cancer related indications associated with expression of a tumor antigen as described herein include, but are not limited to, e.g., autoimmune disease, (e.g., lupus), inflammatory disorders (allergy and asthma) and transplantation. In some embodiments, the tumor antigen-expressing cells express, or at any time expressed, mRNA encoding the tumor antigen. In some embodiments, the tumor antigen-expressing cells produce the tumor antigen protein (e.g., wild-type or mutant), and the tumor antigen protein may be present at normal levels or reduced levels. In some embodiments, the tumor antigen-expressing cells produced detectable levels of a tumor antigen protein at one point, and subsequently produced substantially no detectable tumor antigen protein.

The term “stimulation,” refers to a primary response induced by binding of a stimulatory molecule (e.g., a TCR/CD3 complex or CAR) with its cognate ligand (or tumor antigen in the case of a CAR) thereby mediating a signal transduction event, such as, but not limited to, signal transduction via the TCR/CD3 complex or signal transduction via the appropriate NK receptor or signaling domains of the CAR. Stimulation can mediate altered expression of certain molecules.

The term “stimulatory molecule,” refers to a molecule expressed by an immune cell (e.g., T cell, NK cell, B cell) that provides the cytoplasmic signaling sequence(s) that regulate activation of the immune cell in a stimulatory way for at least some aspect of the immune cell signaling pathway. In some aspects, the-signal is a primary signal that is initiated by, for instance, binding of a TCR/CD3 complex with an MHC molecule loaded with peptide, and which leads to mediation of a T cell response, including, but not limited to, proliferation, activation, differentiation, and the like. A primary cytoplasmic signaling sequence (also referred to as a “primary signaling domain”) that acts in a stimulatory manner may contain a signaling motif which is known as immunoreceptor tyrosine-based activation motif or ITAM. Examples of an ITAM containing-cytoplasmic signaling sequence that is of particular use in the invention include, but are not limited to, those derived from CD3 zeta, common FcR gamma (FCER1G), Fc gamma RIIa, FcR beta (Fc Epsilon Rib), CD3 gamma, CD3 delta, CD3 epsilon, CD79a, CD79b, DAP10, and DAP12. In a specific CAR of the invention, the intracellular signaling domain in any one or more CARS of the invention comprises an intracellular signaling sequence, e.g., a primary signaling sequence of CD3-zeta. In a specific CAR of the invention, the primary signaling sequence of CD3-zeta is a sequence provided as SEQ ID NO: 18, or the equivalent residues from a non-human species, e.g., mouse, rodent, monkey, ape and the like. In a specific CAR of the invention, the primary signaling sequence of CD3-zeta is the sequence as provided in SEQ ID NO:20, or the equivalent residues from a non-human species, e.g., mouse, rodent, monkey, ape, and the like.

The term “Fc silent” refers to an Fc domain that has been modified to have minimal interaction with effector cells. Silenced effector functions may be obtained by mutation in the Fc region of the antibodies and have been described in the art, such as, but not limited to, LALA and N297A (Strohl, W., 2009, Curr. Opin. Biotechnol. vol. 20(6):685-691); and D265A (Baudino et al., 2008, J. Immunol. 181: 6664-69) see also Heusser et al., WO2012065950. Examples of Fc silencing mutations include the LALA mutant comprising L234A and L235A mutation in the IgG1 Fc amino acid sequence, DAPA (D265A, P329A) (see, e.g., U.S. Pat. No. 6,737,056), N297A, DANAPA (D265A, N297A, and P329A), and/or LALADANAPS (L234A, L235A, D265A, N297A and P331S), numbered according to the Eu numbering system. Further, non-limiting exemplary embodiments of silencing mutations include LALAGA (L234A, L235A, and G237A), LALASKPA (L234A, L235A, S267K, and P329A), DAPASK (D265A, P329A, and S267K), GADAPA (G237A, D265A, and P329A), GADAPASK (G237A, D265A, P329A, and S267K), LALAPG (L234A, L235A, and P329G), and LALAPA (L234A, L235A, and P329A), numbered according to the Eu numbering system. Unless otherwise specified herein, numbering of amino acid residues in the Fc region or constant region is according to the Eu numbering system, also called the Eu index, as described in Kabat, et al., Sequences of Proteins of Immunological Interest, 5th Ed. Public Health Service, National Institutes of Health, Bethesda, Md. (1991).

The term “CD3/TCR complex” refers to a complex on the T-cell surface comprising a TCR with including a TCR alpha and TCR beta chain; CD3 including one CD3 gamma chain, one CD3 delta chain, and two CD3 epsilon chains; and a zeta domain. UniProt accession numbers P01848 (TCR alpha, constant domain), P01850 (TCR beta, constant domain 1), A0A5B9 (TCR beta, constant domain 2), P09693 (CD3 gamma), P04234 (CD3 delta), P07766 (CD3 epsilon) provide exemplary human sequences for these chains, with the exception of the zeta chain, responsible for intracellular signaling, which is discussed in further detail below. Further relevant accession numbers include A0A075B662 (murine TCR alpha, constant domain), A0A0A6YWV4 and/or A0A075B5J3 (murine TCR beta, constant domain 1), A0A075B5J4 (murine TCR beta, constant domain 2), P11942 (murine CD3 gamma), P04235 (murine CD3 delta), P22646 (murine CD3 epsilon).

The term “CD28” refers to a T-cell specific glycoprotein CD28, also referred to as Tp44, as well as all alternate names thereof, which functions as a costimulatory molecule. UniProt accession number P10747 provides exemplary human CD28 amino acid sequences (see also HGNC: 1653, Entrez Gene: 940, Ensembl: ENSG00000178562, and OMIM: 186760). Further relevant CD28 sequences include UniProt accession number P21041 (murine CD28).

The term “ICOS” refers to inducible T-cell costimulator, also referred to as AILIM, CVID1, CD278, as well as all alternate names thereof, which functions as a costimulatory molecule. UniProt accession number Q9Y6W8 provides exemplary human ICOS amino acid sequences (see also HGNC: 5351, Entrez Gene: 29851, Ensembl: ENSG00000163600, and OMIM: 604558). Further relevant ICOS sequences include UniProt accession number Q9WVS0 (murine ICOS).

The term “CD27” refers to T-cell activation antigen CD27, Tumor necrosis factor receptor superfamily member 7, T14, T-cell activation antigen S152, Tp55, as well as alternate names thereof, which functions as a costimulatory molecule. UniProt accession number P26842 provides exemplary human CD27 amino acid sequences (see also HGNC: 11922, Entrez Gene: 939, Ensembl: ENSG00000139193, and OMIM: 186711). Further relevant CD27 sequences include UniProt accession number P41272 (murine CD27). The term “CD25” refers to IL-2 subunit alpha, TAC antigen, p55, insulin dependent diabetes mellitus 10, EID21, P55, TCGFR, as well as alternate names thereof, which functions as a growth factor receptor. UniProt accession number P01589 provides exemplary human CD25 amino acid sequences (see also HGNC: 6008, Entrez Gene: 3559, Ensembl: ENSG00000134460, and OMIM: 147730). Further relevant CD25 sequences include UniProt accession number P01590 (murine CD25).

The term “4-1BB” refers to CD137 or Tumor necrosis factor receptor superfamily member 9, as well as alternate names thereof, which functions as a costimulatory molecule. UniProt accession number Q07011 provides exemplary human 4-1BB amino acid sequences (see also HGNC: 11924, Entrez Gene: 3604, Ensembl: ENSG00000049249, and OMIM: 602250). Further relevant 4-1BB sequences include UniProt accession number P20334 (murine 4-1BB).

The term “IL6RA” refers to IL-6 receptor subunit alpha or CD126, as well as alternate names thereof, which functions as a growth factor receptor. UniProt accession number P08887 provides exemplary human IL6RA amino acid sequences (see also HGNC: 6019, Entrez Gene: 3570, Ensembl: ENSG00000160712, and OMIM: 147880 Further relevant IL6RA sequences include UniProt accession number P22272 (murine IL6RA).

The term “IL6RB” refers to IL-6 receptor subunit beta or CD130, as well as alternate names thereof, which functions as a growth factor receptor. UniProt accession number P40189 provides exemplary human IL6RB amino acid sequences. Further relevant IL6RB sequences include UniProt accession number Q00560 (murine IL6RB). The term “CD2” refers to T-cell surface antigen T11/Leu-5/CD2, lymphocyte function antigen 2, T11, or erythrocyte/rosette/LFA-3 receptor, as well as alternate names thereof, which functions as a growth factor receptor. UniProt accession number P06729 provides exemplary human CD2 amino acid sequences (see also HGNC: 1639, Entrez Gene: 914, Ensembl: ENSG00000116824, and OMIM: 186990). Further relevant CD2 sequences include UniProt accession number P08920 (murine CD2).

The term “antigen presenting cell” or “APC” refers to an immune system cell such as an accessory cell (e.g., a B-cell, a dendritic cell, and the like) that displays a foreign antigen complexed with major histocompatibility complexes (MHC ©) on its surface. T-cells may recognize these complexes using their T-cell receptors (TCRs). APCs process antigens and present them to T-cells.

An “intracellular signaling domain,” as the term is used herein, refers to an intracellular portion of a molecule. The intracellular signaling domain generates a signal that promotes an immune effector function of the CAR containing cell, e.g., a CART cell. Examples of immune effector function, e.g., in a CART cell, include cytolytic activity and helper activity, including the secretion of cytokines.

In some embodiments, the intracellular signaling domain can comprise a primary intracellular signaling domain. Exemplary primary intracellular signaling domains include those derived from the molecules responsible for primary stimulation, or antigen dependent simulation. In some embodiments, the intracellular signaling domain can comprise a costimulatory intracellular domain. Exemplary costimulatory intracellular signaling domains include those derived from molecules responsible for costimulatory signals, or antigen independent stimulation. For example, in the case of a CART, a primary intracellular signaling domain can comprise a cytoplasmic sequence of a T cell receptor, and a costimulatory intracellular signaling domain can comprise cytoplasmic sequence from co-receptor or costimulatory molecule.

A primary intracellular signaling domain can comprise a signaling motif which is known as an immunoreceptor tyrosine-based activation motif or ITAM. Examples of ITAM containing primary cytoplasmic signaling sequences include, but are not limited to, those derived from CD3 zeta, common FcR gamma (FCER1G), Fc gamma RIIa, FcR beta (Fc Epsilon Rib), CD3 gamma, CD3 delta, CD3 epsilon, CD79a, CD79b, DAP10, and DAP12.

The term “zeta” or alternatively “zeta chain”, “CD3-zeta” or “TCR-zeta” refers to CD247. Swiss-Prot accession number P20963 provides exemplary human CD3 zeta amino acid sequences. A “zeta stimulatory domain” or alternatively a “CD3-zeta stimulatory domain” or a “TCR-zeta stimulatory domain” refers to a stimulatory domain of CD3-zeta or a variant thereof (e.g., a molecule having mutations, e.g., point mutations, fragments, insertions, or deletions). In some embodiments, the cytoplasmic domain of zeta comprises residues 52 through 164 of GenBank Acc. No. BAG36664.1 or a variant thereof (e.g., a molecule having mutations, e.g., point mutations, fragments, insertions, or deletions). In some embodiments, the “zeta stimulatory domain” or a “CD3-zeta stimulatory domain” is a sequence provided as SEQ ID NO: 9 or 10, or a variant thereof (e.g., a molecule having mutations, e.g., point mutations, fragments, insertions, or deletions). Alternatively or in addition, the term “zeta” or alternatively “zeta chain”, “CD3-zeta” (or “CD3zeta, CD3 zeta or CD3z) or “TCR-zeta” is defined as the protein provided as GenBan Acc. No. BAG36664.1, or the equivalent residues from a non-human species, e.g., mouse, rodent, monkey, ape and the like, and a “zeta stimulatory domain” or alternatively a “CD3-zeta stimulatory domain” or a “TCR-zeta stimulatory domain” is defined as the amino acid residues from the cytoplasmic domain of the zeta chain, or functional derivatives thereof, that are sufficient to functionally transmit an initial signal necessary for T cell activation. In some aspects the cytoplasmic domain of zeta comprises residues 52 through 164 of GenBank Acc. No. BAG36664.1 or the equivalent residues from a non-human species, e.g., mouse, rodent, monkey, ape and the like, that are functional orthologs thereof In some aspects, the “zeta stimulatory domain” or a “CD3-zeta stimulatory domain” is a sequence provided as SEQ ID NO:18. In some aspects, the “zeta stimulatory domain” or a “CD3-zeta stimulatory domain” is a sequence provided as SEQ ID NO:20.

The term a “costimulatory molecule” refers to a cognate binding partner on a T cell that specifically binds with a costimulatory ligand, thereby mediating a costimulatory response by the T cell, such as, but not limited to, proliferation. Costimulatory molecules are cell surface molecules other than antigen receptors or their ligands that are contribute to an efficient immune response. Costimulatory molecules include, but are not limited to, an MHC class I molecule, BTLA and a Toll ligand receptor, as well as OX40, CD27, CD28, CDS, ICAM-1, LFA-1 (CD11a/CD18), ICOS (CD278), and 4-1BB (CD137). Further examples of such costimulatory molecules include CDS, ICAM-1, GITR, BAFFR, HVEM (LIGHTR), SLAMF7, NKp80 (KLRF1), NKp44, NKp30, NKp46, CD160, CD19, CD4, CD8alpha, CD8beta, IL2R beta, IL2R gamma, IL7R alpha, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, NKG2D, NKG2C, TNFR2, TRANCE/RANKL, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, CD19a, and a ligand that specifically binds with CD83.

A costimulatory intracellular signaling domain can be the intracellular portion of a costimulatory molecule. A costimulatory molecule can be represented in the following protein families: TNF receptor proteins, Immunoglobulin-like proteins, cytokine receptors, integrins, signaling lymphocytic activation molecules (SLAM proteins), and activating NK cell receptors. Examples of such molecules include CD27, CD28, 4-1BB (CD137), OX40, GITR, CD30, CD40, ICOS, BAFFR, HVEM, ICAM-1, lymphocyte function-associated antigen-1 (LFA-1), CD2, CDS, CD7, CD287, LIGHT, NKG2C, NKG2D, SLAMF7, NKp80, NKp30, NKp44, NKp46, CD160, B7-H3, and a ligand that specifically binds with CD83, and the like. The intracellular signaling domain can comprise the entire intracellular portion, or the entire native intracellular signaling domain, of the molecule from which it is derived, or a functional fragment or derivative thereof.

“Immune effector cell,” as that term is used herein, refers to a cell that is involved in an immune response, e.g., in the promotion of an immune effector response. Examples of immune effector cells include T cells, e.g., alpha/beta T cells and gamma/delta T cells, B cells, natural killer (NK) cells, natural killer T (NKT) cells, mast cells, and myeloid-derived phagocytes. “Immune effector function or immune effector response,” as that term is used herein, refers to function or response, e.g., of an immune effector cell, that enhances or promotes an immune attack of a target cell. E.g., an immune effector function or response refers a property of a T or NK cell that promotes killing or inhibition of growth or proliferation, of a target cell. In the case of a T cell, primary stimulation and co-stimulation are examples of immune effector function or response.

The term “encoding” or “encode” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (e.g., rRNA, tRNA and mRNA) or a defined sequence of amino acids and the biological properties resulting therefrom. Thus, a gene, cDNA, or RNA, encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

Unless otherwise specified, a “nucleotide sequence encoding an amino acid sequence” includes all nucleotide sequences that are degenerate versions of each other and that encode the same amino acid sequence. The phrase nucleotide sequence that encodes a protein or a RNA may also include introns to the extent that the nucleotide sequence encoding the protein may in some version contain an intron(s).

The term “effective amount” or “therapeutically effective amount” are used interchangeably herein, and refer to an amount of a compound, formulation, material, or composition, as described herein effective to achieve a particular biological result. The term “endogenous” refers to any material from or produced inside an organism, cell, tissue or system.

The term “exogenous” refers to any material introduced from or produced outside an organism, cell, tissue or system.

The term “expression” refers to the transcription and/or translation of a particular nucleotide sequence driven by a promoter.

As used herein the term “extended release agent” refers to an agent that releases a given composition, e.g., a viral vector (e.g., a lentiviral vector) and/or a cell activation agent, over a longer period of time than a comparable immediate release formulation. In some embodiments, the extended release agent is formulated for administration by injection.

The term “transfer vector” refers to a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “transfer vector” includes an autonomously replicating plasmid or a virus. The term should also be construed to further include non-plasmid and non-viral compounds which facilitate transfer of nucleic acid into cells, such as, for example, a polylysine compound, liposome, and the like. Examples of viral transfer vectors include, but are not limited to, adenoviral vectors, adeno-associated virus vectors, retroviral vectors, lentiviral vectors, and the like.

The term “expression vector” refers to a vector comprising a recombinant polynucleotide comprising an expression control sequence operatively linked to a nucleotide sequence to be expressed. An expression vector comprises sufficient cis-acting elements for expression; other elements for expression can be supplied by the host cell or in an in vitro expression system. Expression vectors include all those known in the art, including cosmids, plasmids (e.g., naked or contained in liposomes) and viruses (e.g., lentiviruses, retroviruses, adenoviruses, and adeno-associated viruses, “viral vectors”) that incorporate the recombinant polynucleotide.

The term “lentivirus” refers to a genus of the Retroviridae family. Lentiviruses are unique among the retroviruses in being able to infect non-dividing cells; they can deliver a significant amount of genetic information into the DNA of the host cell, so they are one of the most efficient methods of a gene delivery vector. HIV, SIV, and FIV are all examples of lentiviruses.

The term “lentiviral vector” refers to a vector derived from at least a portion of a lentivirus genome, including especially a self-inactivating lentiviral vector as provided in Milone et al., Mol. Ther. 17(8): 1453-1464 (2009). Other examples of lentivirus vectors that may be used in the clinic, include but are not limited to, e.g., the LENTIVECTOR® gene delivery technology from Oxford BioMedica, the LENTIMAX™ vector system from Lentigen and the like. Nonclinical types of lentiviral vectors are also available and would be known to one skilled in the art.

The term “homologous” or “identity” refers to the subunit sequence identity between two polymeric molecules, e.g., between two nucleic acid molecules, such as, two DNA molecules or two RNA molecules, or between two polypeptide molecules. When a subunit position in both of the two molecules is occupied by the same monomeric subunit; e.g., if a position in each of two DNA molecules is occupied by adenine, then they are homologous or identical at that position. The homology between two sequences is a direct function of the number of matching or homologous positions; e.g., if half (e.g., five positions in a polymer ten subunits in length) of the positions in two sequences are homologous, the two sequences are 50% homologous; if 90% of the positions (e.g., 9 of 10), are matched or homologous, the two sequences are 90% homologous.

“Humanized” forms of non-human (e.g., murine) antibodies are chimeric immunoglobulins, immunoglobulin chains or fragments thereof (such as Fv, Fab, Fab QF(ab, or other antigen-binding subsequences of antibodies) which contain minimal sequence derived from non-human immunoglobulin. For the most part, humanized antibodies and antibody fragments thereof are human immunoglobulins (recipient antibody or antibody fragment) in which residues from a complementary-determining region (CDR) of the recipient are replaced by residues from a CDR of a non-human species (donor antibody) such as mouse, rat or rabbit having the desired specificity, affinity, and capacity. In some instances, Fv framework region (FR) residues of the human immunoglobulin are replaced by corresponding non-human residues. Furthermore, a humanized antibody/antibody fragment can comprise residues which are found neither in the recipient antibody nor in the imported CDR or framework sequences. These modifications can further refine and optimize antibody or antibody fragment performance. In general, the humanized antibody or antibody fragment thereof will comprise substantially all of at least one, and typically two, variable domains, in which all or substantially all of the CDR regions correspond to those of a non-human immunoglobulin and all or a significant portion of the FR regions are those of a human immunoglobulin sequence. The humanized antibody or antibody fragment can also comprise at least a portion of an immunoglobulin constant region (Fc), typically that of a human immunoglobulin. For further details, see Jones et al., Nature, 321: 522-525, 1986; Reichmann et al., Nature, 332: 323-329, 1988; Presta, Curr. Op. Struct. Biol., 2: 593-596, 1992.

“Fully human” refers to an immunoglobulin, such as an antibody or antibody fragment, where the whole molecule is of human origin or consists of an amino acid sequence identical to a human form of the antibody or immunoglobulin.

The term “isolated” means altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell.

The term “operably linked” or “transcriptional control” refers to functional linkage between a regulatory sequence and a heterologous nucleic acid sequence resulting in expression of the latter. For example, a first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Operably linked DNA sequences can be contiguous with each other and, e.g., where necessary to join two protein coding regions, are in the same reading frame.

The term “nucleic acid” or “polynucleotide” refers to deoxyribonucleic acids (DNA) or ribonucleic acids (RNA) and polymers thereof in either single- or double-stranded form. Unless specifically limited, the term encompasses nucleic acids containing known analogues of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions), alleles, orthologs, SNPs, and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); and Rossolini et al., Mol. Cell. Probes 8:91-98 (1994)).

The terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. A polypeptide includes a natural peptide, a recombinant peptide, or a combination thereof.

The term “promoter” refers to a DNA sequence recognized by the synthetic machinery of the cell, or introduced synthetic machinery, required to initiate the specific transcription of a polynucleotide sequence.

The term “promoter/regulatory sequence” refers to a nucleic acid sequence which is required for expression of a gene product operably linked to the promoter/regulatory sequence. In some instances, this sequence may be the core promoter sequence and in other instances, this sequence may also include an enhancer sequence and other regulatory elements which are required for expression of the gene product. The promoter/regulatory sequence may, for example, be one which expresses the gene product in a tissue specific manner.

The term “constitutive promoter” refers to a nucleotide sequence which, when operably linked with a polynucleotide which encodes or specifies a gene product, causes the gene product to be produced in a cell under most or all physiological conditions of the cell.

The term “inducible promoter” refers to a nucleotide sequence which, when operably linked with a polynucleotide which encodes or specifies a gene product, causes the gene product to be produced in a cell substantially only when an inducer which corresponds to the promoter is present in the cell.

The term “tissue-specific promoter” refers to a nucleotide sequence which, when operably linked with a polynucleotide encodes or specified by a gene, causes the gene product to be produced in a cell substantially only if the cell is a cell of the tissue type corresponding to the promoter.

The terms “cancer associated antigen” or “tumor antigen” interchangeably refers to a molecule (typically a protein, carbohydrate or lipid) that is expressed on the surface of a cancer cell, either entirely or as a fragment (e.g., MHC/peptide), and which is useful for the preferential targeting of a pharmacological agent to the cancer cell. In some embodiments, a tumor antigen is a marker expressed by both normal cells and cancer cells, e.g., a lineage marker, e.g., CD19 on B cells. In some embodiments, a tumor antigen is a cell surface molecule that is overexpressed in a cancer cell in comparison to a normal cell, for instance, 1-fold over expression, 2-fold overexpression, 3-fold overexpression or more in comparison to a normal cell. In some embodiments, a tumor antigen is a cell surface molecule that is inappropriately synthesized in the cancer cell, for instance, a molecule that contains deletions, additions or mutations in comparison to the molecule expressed on a normal cell. In some embodiments, a tumor antigen will be expressed exclusively on the cell surface of a cancer cell, entirely or as a fragment (e.g., MHC/peptide), and not synthesized or expressed on the surface of a normal cell. In some embodiments, the CARs of the present invention include CARs comprising an antigen binding domain (e.g., antibody or antibody fragment) that binds to a MHC presented peptide. Normally, peptides derived from endogenous proteins fill the pockets of Major histocompatibility complex (MHC) class I molecules and are recognized by T cell receptors (TCRs) on CD8+ T lymphocytes. The MHC class I complexes are constitutively expressed by all nucleated cells. In cancer, virus-specific and/or tumor-specific peptide/MHC complexes represent a unique class of cell surface targets for immunotherapy. TCR-like antibodies targeting peptides derived from viral or tumor antigens in the context of human leukocyte antigen (HLA)-A1 or HLA-A2 have been described (see, e.g., Sastry et al., J Virol. 2011 85(5):1935-1942; Sergeeva et al., Blood, 2011 117(16):4262-4272; Verma et al., J Immunol 2010 184(4):2156-2165; Willemsen et al., Gene Ther 2001 8(21):1601-1608; Dao et al., Sci Transl Med 2013 5(176):176ra33; Tassev et al., Cancer Gene Ther 2012 19(2):84-100). For example, TCR-like antibody can be identified from screening a library, such as a human scFv phage displayed library.

The term “tumor-supporting antigen” or “cancer-supporting antigen” interchangeably refer to a molecule (typically a protein, carbohydrate or lipid) that is expressed on the surface of a cell that is, itself, not cancerous, but supports the cancer cells, e.g., by promoting their growth or survival e.g., resistance to immune cells. Exemplary cells of this type include stromal cells and myeloid-derived suppressor cells (MDSCs). The tumor-supporting antigen itself need not play a role in supporting the tumor cells so long as the antigen is present on a cell that supports cancer cells.

As used herein, “in vitro transcribed RNA” refers to RNA, preferably mRNA, that has been synthesized in vitro. Generally, the in vitro transcribed RNA is generated from an in vitro transcription vector. The in vitro transcription vector comprises a template that is used to generate the in vitro transcribed RNA.

As used herein, a “poly(A)” is a series of adenosines attached by polyadenylation to the mRNA. In a preferred embodiment of a construct for transient expression, the polyA is between 50 and 5000 (SEQ ID NO: 34), preferably greater than 64, more preferably greater than 100, most preferably greater than 300 or 400. poly(A) sequences can be modified chemically or enzymatically to modulate mRNA functionality such as localization, stability or efficiency of translation.

As used herein in connection with expression, e.g., expression of a CAR molecule, “transient” refers to expression of a non-integrated transgene for a period of hours, days or weeks, wherein the period of time of expression is less than the period of time for expression of the gene if integrated into the genome or contained within a stable plasmid replicon in the host cell.

As used herein, the terms “treat”, “treatment” and “treating” refer to the reduction or amelioration of the progression, severity and/or duration of a proliferative disorder, or the amelioration of one or more symptoms (preferably, one or more discernible symptoms) of a proliferative disorder resulting from the administration of one or more therapies (e.g., one or more therapeutic agents such as a CAR of the invention). In specific embodiments, the terms “treat”, “treatment” and “treating” refer to the amelioration of at least one measurable physical parameter of a proliferative disorder, such as growth of a tumor, not necessarily discernible by the patient. In other embodiments the terms “treat”, “treatment” and “treating”-refer to the inhibition of the progression of a proliferative disorder, either physically by, e.g., stabilization of a discernible symptom, physiologically by, e.g., stabilization of a physical parameter, or both. In other embodiments the terms “treat”, “treatment” and “treating” refer to the reduction or stabilization of tumor size or cancerous cell count.

The term “signal transduction pathway” refers to the biochemical relationship between a variety of signal transduction molecules that play a role in the transmission of a signal from one portion of a cell to another portion of a cell. The phrase “cell surface receptor” includes molecules and complexes of molecules capable of receiving a signal and transmitting signal across the membrane of a cell.

The term “subject” is intended to include living organisms in which an immune response can be elicited (e.g., mammals, human).

The term, a “substantially purified” cell refers to a cell that is essentially free of other cell types. A substantially purified cell also refers to a cell which has been separated from other cell types with which it is normally associated in its naturally occurring state. In some instances, a population of substantially purified cells refers to a homogenous population of cells. In other instances, this term refers simply to cell that have been separated from the cells with which they are naturally associated in their natural state. In some aspects, the cells are cultured in vitro. In other aspects, the cells are not cultured in vitro.

The term “therapeutic” as used herein means a treatment. A therapeutic effect is obtained by reduction, suppression, remission, or eradication of a disease state.

The term “prophylaxis” as used herein means the prevention of or protective treatment for a disease or disease state.

The term “transfected” or “transformed” or “transduced” refers to a process by which exogenous nucleic acid is transferred or introduced into the host cell. A “transfected” or “transformed” or “transduced” cell is one which has been transfected, transformed or transduced with exogenous nucleic acid. The cell includes the primary subject cell and its progeny.

The term “specifically binds,” refers to an antibody, or a ligand, which recognizes and binds with a binding partner (e.g., a tumor antigen) protein present in a sample, but which antibody or ligand does not substantially recognize or bind other molecules in the sample.

“Refractory” as used herein refers to a disease, e.g., cancer, that does not respond to a treatment. In embodiments, a refractory cancer can be resistant to a treatment before or at the beginning of the treatment. In other embodiments, the refractory cancer can become resistant during a treatment. A refractory cancer is also called a resistant cancer.

“Relapsed” as used herein refers to the return of a disease (e.g., cancer) or the signs and symptoms of a disease such as cancer after a period of improvement, e.g., after prior treatment of a therapy, e.g., cancer therapy

A “system” as the term is used herein in connection with, for example, gene editing, refers to a group of molecules, e.g., one or more molecules, which together act to produce a desired function.

A “gene editing system” as the term is used herein, refers to a system, e.g., one or more molecules, that direct and effect an alteration, e.g., a deletion, of one or more nucleic acids at or near a site of genomic DNA targeted by said system. Gene editing systems are known in the art, and are described more fully below.

A “dominant negative” gene product or protein is one that interferes with the function of a gene product or protein. The gene product affected can be the same or different from the dominant negative protein. Dominant negative gene products can be of many forms, including truncations, full length proteins with point mutations or fragments thereof, or fusions of full-length wild type or mutant proteins or fragments thereof with other proteins. The level of inhibition observed can be very low. For example, it may require a large excess of the dominant negative protein compared to the functional protein or proteins involved in a process in order to see an effect. It may be difficult to see effects under normal biological assay conditions.

The term “proportion” refers to the ratio of the specified molecule to the total number of molecules in a population. In an exemplary embodiment, a proportion of T cells having a specific phenotype (e.g., T_{SCM} cells) refers to the ratio of the number of T cells having that phenotype relative to the total number of T cells in a population. In an exemplary embodiment, a proportion of T cells having a specific phenotype (e.g., CD45RA+CD62L+ cells) refers to the ratio of the number of T cells having that phenotype relative to the total number of T cells in a population. It will be understood that such proportions may be measured against certain subsets of cells, where indicted. For example, the proportion of CD4+ T_{SCM} cells may be measured against the total number of CD4+ T cells.

The term “population of immune effector cells” as used herein refers to a composition comprising at least two, e.g., two or more, e.g., more than one, immune effector cell, and does not denote any level of purity or the presence or absence of other cell types. In an exemplary embodiment, the population is substantially free of other cell types. In another exemplary embodiment, the population comprises at least two cells of the specified cell type or having the specified function or property.

As used herein, the term “biomaterial” refers to a substance engineered to interact with a biological system for a therapeutic purpose. A “hydrogel” is such a substance comprised of a network of polymer chain that may be hydrated to adopt a gel form—typically as a result of cross-linking between the polymer chains. A “cryogel” is a form of hydrogel that has been formed by freezing. In some embodiments, a cryogel is formed by allowing crosslinking to occur in a partially frozen state, resulting in a hydrogel network.

The terms “T_{SCM}-like cell,” “naive T Cell’ and “naïve T cell” are used interchangeably and refer to a less differentiated T cell state, that is characterized by surface expression of CD45RA and CD62L (e.g., is CD45RA positive and CD62L positive (sometimes written as CD45RA+CD62L+)). In general, T cell differentiation proceeds, from most “naive” to most “exhausted,” T_{SCM}-like (e.g., a CD45RA+CD62L+ cell)>T_{CM} (e.g., a CD45RA-CD62L+ cell)>T_{EM} (e.g., a CD45RA−CD62L− cell)>T_{EFF}. Naive T cells may be characterized, for example, as having increased self-renewal, anti-tumor efficacy, proliferation and/or survival, relative to a more exhausted T cell phenotype. In an exemplary embodiment, a naive T cell refers to a CD45RA+CD62L+ T cell. In another exemplary embodiment, a naive T cell refers to a T_{SCM} cell, e.g., a CD45RA+CD62L+CCR7+CD27+CD95+ T cell.

The term “T_{SCM}” refers to a T cell having a stem cell memory phenotype, characterized in that it expresses CD45RA, CD62L, CCR7, CD27 and CD95 on its cell surface (e.g., is CD45RA positive, CD62L positive, CCR7 positive, CD27 positive and CD95 positive (sometimes written as CD45RA+CD62L+CCR7+CD27+CD95+)). A T_{SCM} cell is an example of a naive T cell. The T cell may be CD4+ and/or CD8+ T cell.

For specific proteins described herein (e.g., VEGF-C), the named protein includes any of the protein naturally occurring forms, variants or homologs (e.g., within at least 50%, 80%, 90%, 95%, 96%, 97%, 98%, 99% or 100% activity compared to the native protein). In some embodiments, variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring form. In some embodiments, the protein is the protein as identified by its NCBI sequence reference (e.g., NP_005420.1). In some embodiments, the protein is the protein as identified by its NCBI sequence reference, homolog or functional fragment thereof.

As used herein, the term “alkyl” refers to a fully saturated branched or unbranched (or straight chain or linear) hydrocarbon moiety, comprising 1 to 20 carbon atoms. Preferably the alkyl comprises 1 to 6 carbon atoms, and more preferably 1 to 4 carbon atoms. Representative examples of alkyl include methyl, ethyl, n-propyl, iso-propyl, n-butyl, sec-butyl, iso-butyl, vert-butyl, n-pentyl, isopentyl, neopentyl, n-hexyl, 3-methylhexyl, 2,2-dimethylpentyl, 2,3-dimethylpentyl, n-heptyl. For example, the term “C₁₋₆alkyl” refers to a hydrocarbon having from one to six carbon atoms, and the term “C₁₋₇alkyl” refers to a hydrocarbon having from one to seven carbon atoms.

As used herein, the term “haloalkyl” refers to an alkyl as defined herein, that is substituted by one or more halo groups as defined herein. Preferably the haloalkyl can be monohaloalkyl, dihaloalkyl or polyhaloalkyl including perhaloalkyl. A monohaloalkyl can have one iodo, bromo, chloro or fluoro within the alkyl group. Dihaloalky and polyhaloalkyl groups can have two or more of the same halo atoms or a combination of different halo groups within the alkyl. Preferably, the polyhaloalkyl contains up to 12, or 10, or 8, or 6, or 4, or 3, or 2 halo groups. Representative examples of haloalkyl are fluoromethyl, difluoromethyl, trifluoromethyl, chloromethyl, dichloromethyl, trichloromethyl, pentafluoroethyl, heptafluoropropyl, difluorochloromethyl, dichlorofluoromethyl, difluoroethyl, difluoropropyl, dichloroethyl and dichloropropyl. A perhaloalkyl refers to an alkyl having all hydrogen atoms replaced with halo atoms. For example, the term “halo-C₁₋₆alkyl” refers to a hydrocarbon having one to six carbon atoms and being substituted by one or more halo groups, and the term “halo-C₁₋₇alkyl” refers to a hydrocarbon having one to seven carbon atoms and being substituted by one or more halo groups.

As used herein, “salts” includes pharmaceutically acceptable acid addition salts that can be formed with inorganic acids and organic acids, e.g., acetate, aspartate, benzoate, besylate, bromide/hydrobromide, bicarbonate/carbonate, bisulfate/sulfate, camphorsulformate, chloride/hydrochloride, chlortheophyllonate, citrate, ethandisulfonate, fumarate, gluceptate, gluconate, glucuronate, hippurate, hydroiodide/iodide, isethionate, lactate, lactobionate, laurylsulfate, malate, maleate, malonate, mandelate, mesylate, methylsulphate, naphthoate, napsylate, nicotinate, nitrate, octadecanoate, oleate, oxalate, palmitate, pamoate, phosphate/hydrogen phosphate/dihydrogen phosphate, polygalacturonate, propionate, stearate, succinate, sulfosalicylate, tartrate, tosylate and trifluoroacetate salts.

Inorganic acids from which salts can be derived include, for example, hydrochloric acid, hydrobromic acid, sulfuric acid, nitric acid, phosphoric acid, and the like. Organic acids from which salts can be derived include, for example, acetic acid, propionic acid, glycolic acid, oxalic acid, maleic acid, malonic acid, succinic acid, fumaric acid, tartaric acid, citric acid, benzoic acid, mandelic acid, methanesulfonic acid, ethanesulfonic acid, toluenesulfonic acid, sulfosalicylic acid, and the like. Pharmaceutically acceptable base addition salts can be formed with inorganic and organic bases.

Inorganic bases from which salts can be derived include, for example, ammonium salts and metals from columns I to XII of the periodic table. In certain embodiments, the salts are derived from sodium, potassium, ammonium, calcium, magnesium, iron, silver, zinc, and copper; particularly suitable salts include ammonium, potassium, sodium, calcium and magnesium salts. Organic bases from which salts can be derived include, for example, primary, secondary, and tertiary amines, substituted amines including naturally occurring substituted amines, cyclic amines, basic ion exchange resins, and the like. Certain organic amines include isopropylamine, benzathine, cholinate, diethanolamine, diethylamine, lysine, meglumine, piperazine and tromethamine.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. As another example, a range such as 95-99% identity, includes something with 95%, 96%, 97%, 98% or 99% identity, and includes subranges such as 96-99%, 96-98%, 96-97%, 97-99%, 97-98% and 98-99% identity. This applies regardless of the breadth of the range.

The terms “B cell antigen” or “B-Cell antigen” are used interchangeably, and refer to a molecule (typically a protein, carbohydrate or lipid) that is preferentially or specifically expressed on the surface of a B cell which can be targeted with an agent which binds thereto. The B cell antigen of particular interest is preferentially expressed on B cells compared to other non-B cell tissues of a mammal. The B cell antigen may be expressed on one particular B cell population, e.g., B cell precursors or mature B cells, or on more than one particular B cell population, e.g., both precursor B cells and mature B cells. Exemplary B cell surface markers include: CD5, CD10, CD19, CD20, CD21, CD22, CD23, CD24, CD25, CD27, CD30, CD34, CD37, CD38, CD40, CD53, CD69, CD72, CD73, CD74, CD75, CD77, CD79a, CD79b, CD80, CD81, CD82, CD83, CD84, CD85, CD86, CD123, CD135, CD138, CD179, CD269, Flt3, ROR1, BCMA, FcRn5, FcRn2, CS-1, CXCR4, 5, 7, IL-7/3R, 1L7/4/3R, and IL4R. In some embodiments, the B-Cell antigen is: CD19, CD20, CD22, FcRn5, FcRn2, BCMA, CS-1 or CD138. In embodiments, the B-Cell antigen is CD19. In embodiments, the B-Cell antigen is CD20. In embodiments, the B-Cell antigen is CD22. In embodiments, the B-Cell antigen is BCMA. In embodiments, the B-Cell antigen is FcRn5. In embodiments, the B-Cell antigen is FcRn2. In embodiments, the B-Cell antigen is CS-1. In embodiments, the B-Cell antigen is CD138.

The terms “solid tumor antigen” or “solid tumor cell antigen” refer to a molecule (typically a protein, carbohydrate or lipid) that is preferentially or specifically expressed on the surface of a solid tumor cell which can be targeted with an agent which binds thereto. The solid tumor antigen of particular interest is preferentially expressed on a solid tumor cell compared to other non-tumor tissues of a mammal. The solid tumor antigen may be expressed on one particular solid tumor cell population, e.g., on mesothelioma tumor cells, or on more than one particular solid tumor cell population, e.g., both mesothelioma tumor cells and ovarian cancer cells. Exemplary solid tumor antigens include: EGFRvIII, mesothelin, GD2, Tn Ag, PSMA, TAG72, CD44v6, CEA, EPCAM, KIT, IL-13Ra2, leguman, GD3, CD171, IL-11Ra, PSCA, MAD-CT-1, MAD-CT-2, VEGFR2, LewisY, CD24, PDGFR-beta, SSEA-4, folate receptor alpha, ERBBs (e.g., ERBB2), Her2/neu, MUC1, EGFR, NCAM, Ephrin B2, CAIX, LMP2, sLe, HMWMAA, o-acetyl-GD2, folate receptor beta, TEM1/CD248, TEM7R, FAP, Legumain, HPV E6 or E7, ML-IAP, CLDN6, TSHR, GPRC5D, ALK, Polysialic acid, Fos-related antigen, neutrophil elastase, TRP-2, CYP1B1, sperm protein 17, beta human chorionic gonadotropin, AFP, thyroglobulin, PLAC1, globoH, RAGE1, MN-CA IX, human telomerase reverse transcriptase, intestinal carboxyl esterase, mut hsp 70-2, NA-17, NY-BR-1, UPK2, HAVCR1, ADRB3, PANX3, GPR20, Ly6k, OR51E2, TARP, GFRa4, and a peptide of any of these antigens presented on MHC. In some embodiments, the solid tumor antigen is CLDN6, mesothelin or EGFRvIII.

The terms “myeloid tumor antigen” or “myeloid tumor cell antigen” refer to a molecule (typically a protein, carbohydrate or lipid) that is preferentially or specifically expressed on the surface of a myeloid tumor cell which can be targeted with an agent which binds thereto. The myeloid tumor antigen of particular interest is preferentially expressed on a myeloid tumor cell compared to other non-tumor tissues of a mammal. The myeloid tumor antigen may be expressed on one particular myeloid tumor cell population, e.g., on acute myeloid leukemia (AML) tumor cells, or on more than one particular myeloid tumor cell population. Exemplary myeloid tumor antigens include: CD33 and CLL-1.

The term “antigen of a hematological tumor not of B-Cell lineage” refers to a molecule (typically a protein, carbohydrate or lipid) that is preferentially or specifically expressed on the surface of a tumor or cancer of hematopoietic or lymphoid tissue origin, other than of B-Cell origin. These include tumors of myeloid lineage origin, e.g., tumors derived from granulocyte, erythrocyte, thrombocyte, macrophage and/or mast cell origin, or any of their precursor cell populations, and tumors of lymphoid origin other than B-Cell origin, e.g., T cell, NK cell and/or plasma cell origin, or any of their precursor cell populations.

Headings, sub-headings or numbered or lettered elements, e.g., (a), (b), (i) etc., are presented merely for ease of reading. The use of headings or numbered or lettered elements in this document does not require the steps or elements be performed in alphabetical order or that the steps or elements are necessarily discrete from one another.

All publications, patent applications, patents, and other references mentioned herein are incorporated by reference in their entirety.

Other features, objects, and advantages of the invention will be apparent from the description and drawings, and from the claims.

### Description

Contemplated herein is a first composition comprising a biomaterial and a cell recruitment factor and/or a second composition comprising a viral vector, optionally with a particle, e.g., a mesoporous silica particle (MSP), and a cell activation agent, e.g., a multispecific binding molecule described herein.

Also, contemplated herein is a composition comprising a biomaterial comprising a cell recruitment factor; a first population of mesoporous silica particles; a viral vector; and, optionally, a cell activation agent, and methods of use thereof Also, contemplated herein is a composition and a biomaterial comprising a cell recruitment factor; a first population of mesoporous silica particles; a viral vector; and, optionally, a cell activation agent, and methods of use thereof.

The elements of these compositions and methods of use thereof are described herein below. Not to be bound by theory, Applicants contemplate that the compositions are able to be delivered locally, e.g., under the skin, and through use of a cell recruitment factor, cells are recruited to the defined site of delivery and are able to be transduced by the viral vector. In embodiments where the vector encodes a CAR, the transduced cells are CAR expressing and, thus, may be used to target a particular antigen to treat a disease, disorder, or condition.

Biomaterials

In some embodiments, the biomaterial comprises a hydrogel, optionally a cryogel. In some embodiments, the cryogel comprises gelatin, hyaluronic acid (HA), collagen, alginate, laminin, chitosan, silk fibroin, agarose, poly(ethylene glycol), poly-vinyl alcohol, and/or hydroxyethyl methylacrylate. In some embodiments, the biomaterial comprises an alginate hydrogel, e.g., an alginate cryogel. In some embodiments, the biomaterial comprises a hyaluronic acid hydrogel (HA hydrogel). In some embodiments, the biomaterial comprises a hyaluronic acid cryogel.

In some embodiments, alginate hydrogel, e.g., an alginate cryogel further comprises norbornene and/or tetrazine. In some embodiments, the norbornene and/or tetrazine is covalently associated with, e.g., chemically linked to the alginate. In some embodiments, the norbornene and/or tetrazine is non-covalently associated with, e.g., adsorbed on, the alginate.

In some embodiments, a composition comprising the cryogel, e.g., an alginate cryogel, and a cell recruitment factor further comprises laponite. In some embodiments, a composition comprising the hydrogel, e.g., an HA hydrogel, and a cell recruitment factor further comprises laponite. Without wishing to be bound by theory, in some embodiments, use of laponite allows for a slow and/or controlled release of the cell recruitment factor from the composition. In some embodiments, the laponite is present at a concentration of about 0.1 to about 0.5 mg/mL, e.g., about 0.1 to 0.4 mg/mL, about 0.1 to 0.35 mg/mL, about 0.1 to 0.3 mg/mL, about 0.1 to 0.25 mg/mL, about 0.1 to 0.15 mg/mL, about 0.15 to 0.5 mg/mL, about 0.15 to 0.4 mg/mL, about 0.15 to 0.35 mg/mL, about 0.15 to 0.3 mg/mL, about 0.15 to 0.25 mg/mL, about 1.5 mg/mL to 0.2 mg/mL, about 0.2 to 0.5 mg/mL, about 0.2 to 0.4 mg/mL, about 0.2 to 0.35 mg/mL, about 0.2 to 0.3 mg/mL, about 0.2 to 0.25 mg/mL, about 0.25 to 0.5 mg/mL, about 0.25 to 0.4 mg/mL, about 0.25 to about 0.35 mg/mL, about 0.25 to 0.3 mg/mL, about 0.3 to 0.5 mg/mL, about 0.3 to 0.4 mg/mL, about 0.35 to 0.5 mg/mL, about 0.35 to 0.4 mg/mL, about 0.4 to 0.5 mg/mL, about 0.1 mg/mL, about 0.15 mg/mL, about 0.2 mg/mL, about 0.25 mg/mL, 0.3 mg/mL, about 0.35 mg/mL, or about 0.5 mg/mL. In some embodiments, the laponite is present at a concentration of about 0.25 mg/mL.

In some embodiments, the cryogel comprises pores between about 10 to 300 μm in diameter, e.g., between about 10 to 20 μm, about 10 to 30 μm, about 10 to 40 μm, about 10 to 50 μm, about 10 to 100 μm, about 10 to 150 μm, about 10 to 200 μm, about 10 to 250 μm, about 20 to 30 μm, about 20 to 40 μm, about 20 to 50 μm, about 20 to 100 μm, about 20 to 150 μm, about 20 to 200 μm, about 20 to 250 μm, about 20 to 300 μm, about 50 to 300 μm, about 50 to 100 μm, 50 to about 150 μm, 50 to about 200 μm, 50 to about 250 μm, 100 to about 150 μm, 100 to about 200 μm, 100 to about 250 μm, about 100 to 300 μm, about 150 to 200 μm, about 150 to 250 μm, about 150 to 300 μm, about 200 to 250 μm, about 200 to 300 μm, or about 250 to 300 μm. In some embodiments, the cryogel does not comprise pores. In some embodiments, the cryogel comprises pores of substantially the same size. In some embodiments, the biomaterial comprises pores of different sizes. In some embodiments, the cryogel is chemically crosslinked.

Methods of manufacturing biomaterials are well known in the art. See, e.g., Koshy, S. T., Zhang, D., Grolman, J. M., Stafford, A. G., & Mooney, D. J. (2018). Injectable nanocomposite cryogels for versatile protein drug delivery. Acta biomaterialia, 65, 36-43 (describing the manufacture of an alginate cryogel). Additionally, biomaterials and components thereof may be commercially purchased, e.g., Partek SLC (silica from EMD Millipore) or TruTag silica particles.

In some embodiments, the cryogel, e.g., the alginate cryogel, is administered to a high subcutaneous space or a subcutaneous space adjacent to the dermis. In some embodiments, the hydrogel, e.g., the HA hydrogel, is administered to a high subcutaneous space or a subcutaneous space adjacent to the dermis.

Cell Recruitment Factors

In some embodiments, a cell recruitment factor is to be used in a composition or method described herein.

In some embodiments, the cell recruitment factor induces lymphangiogenesis. In some embodiments, induction of lymphangiogenesis comprises an increase in the level and/or activation of lymphatic endothelial cells (LECs) (for example, CD45−CD31+PDPN+ cells). In some embodiments, the level of LECs (e.g., CD45−CD31+PDPN+ cells) is increased by at least 10%, 20%, 30%, 40%, 50%, 60, 70%, 75%, 80%, 85%, 90%, 95% 100%, or 200%.

In some embodiments, the cell recruitment factor recruits, e.g., selectively recruits immune cells, optionally T-cells and/or NK-cells. In some embodiments, the cell recruitment factor recruits cells (e.g., immune cells, e.g., T cells) directly. In some embodiments, the cell recruitment factor recruits cells (e.g., immune cells, e.g., T cells) indirectly. In some embodiments, the cell recruitment factor induces lymphangiogenesis, which in turn recruits cells, e.g., immune cells, e.g., T cells.

In some embodiments, the cell recruitment factor recruits (for example, directly or indirectly) T cells, e.g., naïve T cells (for example, CD45RA+CD62L+ T cells or CD45RA+CD62L+CCR7+CD27+CD95+ T cells). In some embodiments, recruitment of T cells comprises an increase in the level of T cells. In some embodiments, the level of T cells is increased by at least 10%, 20%, 30%, 40%, 50%, 60, 70%, 75%, 80%, 85%, 90%, 95%, 100%, 200%, 250%, or 300%.

In some embodiments, the cell recruitment factor is selected from the group consisting of CCL19, CXCL9, CXCL10, XCL1, IL-2, IL-7, CCL21, GM-CSF, CCL17, CCL22, CCL20, CCL27, IL-15 (for example, hetIL-15 (IL15/sIL-15Ra)), lymphotoxin alpha, lymphotoxin beta, VEGF-C, FLT3L, G-CSF, PDGF, S100A8/A9, CSF-1, CXCL8, CCL20, CCL17, CCR5, CCR6, CCL2, VEGF, Angiopoietin-2, PGE2, LTB4, CXC3L1, CCL19, CCL21, CXCL10, CXCL11, and/or CXCL12. In some embodiments, the cell recruitment factor comprises VEGF-C or a functional variant thereof.

Without wishing to be bound by theory, in some embodiments, the release of the cell recruitment factor, e.g., a VEGF-C or variant thereof, from a cryogel described herein induces lymphangiogenesis of pre-existing skin lymphatic capillaries, activating lymphatic endothelial cells (LEC), which in turn secrete chemokines such as CCL21, recruiting T cells, e.g., naïve T cells, to the site of administration of the cryogel, e.g., to the site in the dermis on top of the gel. In some embodiments, the time to induce lymphangiogenesis and recruit immune cells, e.g., T cells, by a cell recruitment factor described herein comprises about 5 to about 28 days, e.g., about 5 to 21 days, about 5 to 15 days, about 5 to 14 days, about 5 to 10 days, about 7 to about 28 days, about 7 to about 21 days, about 7 to 15 days, about 7 to 14 days, about 7 to 10 days, about 10 to 28 days, about 10 to 21 days, about 10 to 15 days, about 10 to 14 days, about 14 to 28 days, about 14 to 21 days, about 15 to 28 days, about 15 to 21 days, about 21 to 28 days, about 7 days, about 10 days, about 14 days, about 15 days, or about 20 days, about 21 days, about 28 days. In some embodiments, the time to induce lymphangiogenesis and recruit immune cells, e.g., T cells, by a cell recruitment factor described herein comprises 14 days (e.g., two weeks). In some embodiments, the time to induce lymphangiogenesis and recruit immune cells, e.g., T cells, by a cell recruitment factor described herein comprises 21 days (e.g., three weeks). In some embodiments, the time to induce lymphangiogenesis and recruit immune cells, e.g., T cells, by a cell recruitment factor described herein comprises 28 days (e.g., four weeks or one month).

Methods of producing, formulating, and/or procuring these various cell recruitment factors are known in the art. See, e.g., Leppanen V M, Prota A E, Jeltsch M, et al. Structural determinants of growth factor binding and specificity by VEGF receptor 2. Proc Natl Acad Sci USA. 2010; 107(6):2425-2430. doi:10.1073/pnas.0914318107; Leppänen V M, Tvorogov D, Kisko K, et al. Structural and mechanistic insights into VEGF receptor 3 ligand binding and activation. Proc Natl Acad Sci USA. 2013; 110(32):12960-12965. doi:10.1073/pnas.1301415110; Joyce Chiu, Jason W. H. Wong, Michael Gerometta, and Philip J. Hogg. Mechanism of Dimerization of a Recombinant Mature Vascular Endothelial Growth Factor C Biochemistry 2013 53 (1), 7-9. doi: 10.1021/bi401518b; Broggi, M. A. S., Schmaler, M., Lagarde, N., Rossi, S. W. Isolation of Murine Lymph Node Stromal Cells. J. Vis. Exp. (90), e51803, doi:10.3791/51803 (2014); Fankhauser, M., M. A. Broggi, L. Potin, N. Bordry, L. Jeanbart, A. W. Lund, E. Da Costa, S. Hauert, M. Rincon-Restrepo, and C. Tremblay. 2017. Tumor lymphangiogenesis promotes T cell infiltration and potentiates immunotherapy in melanoma. Science translational medicine. 9:eaa14712; US 2019/0099485 A1: “Lymphangiogenesis for therapeutic immunomodulation” (2017); and Vokali, E., S. Y. Shann, S. Hirosue, M. Rincon-Restrepo, F. V. Duraes, S. Scherer, P. Corthésy-Henrioud, W. W. Kilarski, A. Mondino, and D. Zehn. 2020. Lymphatic endothelial cells prime naïve CD8+ T cells into memory cells under steady-state conditions. Nature communications. 11:1-18.

In some embodiments, the VEGF-C is selected from the group consisting of immature VEGF-C peptide or mature VEGF-C peptide. In some embodiments, the mature VEGF-C peptide is the minor mature form or major mature form. In some embodiments, the mature VEGF-C peptide is a wild type minor mature form or a wild type major mature form. In some embodiments, the mature VEGF-C peptide is a modified minor mature form or a modified major mature form. In some embodiments, the mature VEGF-C peptide is a modified minor mature form comprising a mutation at Cysteine 137 (e.g., C137A) or a modified major mature form comprising a mutation at Cysteine 137 (e.g., C137A), numbered according to SEQ ID NO: 725. In some embodiments, the mature VEGF-C peptide is a modified minor mature form comprising a C137A mutation or a modified major mature form comprising a C137A mutation. In some embodiments, the mature VEGF-C peptide is present as a dimer or monomer. In some embodiments, the VEGF-C is a dimer of the major mature form further comprising a C137A mutation in each monomer. In some embodiments, the VEGF-C is a dimer of the minor mature form further comprising a C137A mutation in each monomer. In some embodiments, the VEGF-C is selected from a sequence provided in Table 18 below. In some embodiments, the VEGF-C is selected from a sequence provided in Table 18 or a sequence having at least about 80%, 85%, 90%, 92%, 95%, 97%, 98%, or 99% sequence identity thereto. In some embodiments, the VEGF-C sequence comprises or does not comprise a linker (e.g., a glycine-serine linker) and/or a his tag.

In some embodiments, the VEGF-C comprises the amino acid sequence of SEQ ID NO: 731, 732, 733, or 734, or a sequence having at least about 80%, 85%, 90%, 92%, 95%, 97%, 98%, or 99% sequence identity thereto. In some embodiments, the VEGF-C comprises the amino acid sequence of SEQ ID NO: 741, or a sequence having at least about 80%, 85%, 90%, 92%, 95%, 97%, 98%, or 99% sequence identity thereto, provided that position 26 is not Cysteine (C), e.g., is Alanine (A). In some embodiments, the VEGF-C comprises the amino acid sequence of SEQ ID NO: 737 or 738, or a sequence having at least about 80%, 85%, 90%, 92%, 95%, 97%, 98%, or 99% sequence identity thereto.

In some embodiments, the VEGF-C comprises the amino acid sequence of SEQ ID NO: 743, or a sequence having at least about 80%, 85%, 90%, 92%, 95%, 97%, 98%, or 99% sequence identity thereto. In some embodiments, the VEGF-C further comprises the amino acid sequence of SEQ ID NO: 740 or a sequence having at least about 80%, 85%, 90%, 92%, 95%, 97%, 98%, or 99% sequence identity thereto.

In some embodiments, the VEGF-C comprises the amino acid sequence of SEQ ID NO: 736 or an amino acid sequence having at least about 80%, 85%, 90%, 92%, 95%, 97%, 98%, or 99% sequence identity thereto. In some embodiments, the VEGF-C comprises the amino acid sequence of SEQ ID NO: 735 or an amino acid sequence having at least about 80%, 85%, 90%, 92%, 95%, 97%, 98%, or 99% sequence identity thereto.

TABLE 18
VEGF-C Variants
The sequences below correspond to a monomer. When a dimer is formed, 2 of the same
sequences are assembled together via cysteine bridges. It is noted that the his tag is used for
experimental purposes but may not be necessary in all embodiments.

| SEQ | | | |
|---|---|---|---|
| ID NO: | DESCRIPTOR | SEQUENCE | KEY |
| 725 | >sp\|P49767\|VEGFC_ | MHLLGFFSVACSLLAAALLPGPREAPAAAAAFE | Underlined: cleaved |
| | HUMAN Vascular | SGLDLSDAEPDAGEATAYASKDLEEQLRSVSS | residues |
| | endothelial growth | VDELMTVLYPEYWKMYKCQLRKGGWQHN | Bold: propeptide |
| | factor C OS = Homo | REQANLNSRTEETIKFAAAHYNTEILKSIDNEW | |
| | sapiens OX = 9606 | RKTQCMPREVCIDVGKEFGVATNTFFKPPCVSV | |
| | GN = VEGFC PE = 1 | YRCGGCCNSEGLQCMNTSTSYLSKTLFEITVPLS | |
| | SV = 1 | QGPKPVTISFANHTSCRCMSKLDVYRQVHSIIRR | |
| | | SLPATLPQCQAANKTCPTNYMWNNHICRCLAQ | |
| | | EDFMFSSDAGDDSTDGFHDICGPNKELDEETCQ | |
| | | CVCRAGLRPASCGPHKELDRNSCQCVCKNKLFP | |
| | | SQCGANREFDENTCQCVCKRTCPRNQPLNPGK | |
| | | CACECTESPQKCLLKGKKFHHQTCSCYRRPCTN | |
| | | RQKACEPGFSYSEEVCRCVPSYWKRPQMS | |
| 727 | >1 immature | FESGLDLSDAEPDAGEATAYASKDLEEQLRSVS | Underlined |
| | peptide | SVDELMTVLYPEYWKMYKCQLRKGGWQHNRE | propeptide |
| | (32-227)- | QANLNSRTEETIKFAAAHYNTEILKSIDNEWRKT | Double underlined: |
| | VEGFC_His | QCMPREVCIDVGKEFGVATNTFFKPPCVSVYRC | GS linker |
| | | GGCCNSEGLQCMNTSTSYLSKTLFEITVPLSQGP | Bold: his tag |
| | | KPVTISFANHTSCRCMSKLDVYRQVHSIIRRGSH | |
| | | HHHHH | |
| 729 | >1 immature | FESGLDLSDAEPDAGEATAYASKDLEEQLRSVS | Underlined: |
| | peptide | SVDELMTVLYPEYWKMYKCQLRKGGWQHNRE | propeptide |
| | (32-227)-VEGFC | QANLNSRTEETIKFAAAHYNTEILKSIDNEWRKT | |
| | | QCMPREVCIDVGKEFGVATNTFFKPPCVSVYRC | |
| | | GGCCNSEGLQCMNTSTSYLSKTLFEITVPLSQGP | |
| | | KPVTISFANHTSCRCMSKLDVYRQVHSIIRR | |
| 731 | >2 major mature | AHYNTEILKSIDNEWRKTQCMPREVCIDVGKEF | Double underlined: |
| | (112-227)- | GVATNTFFKPPCVSVYRCGGCCNSEGLQCMNT | GS linker |
| | VEGFC_His | STSYLSKTLFEITVPLSQGPKPVTISFANHTSCRC | Bold: his tag |
| | | MSKLDVYRQVHSIIRRGSHHHHHH | |
| 732 | >2 major mature | AHYNTEILKSIDNEWRKTQCMPREVCIDVGKEF | |
| | (112-227)-VEGFC | GVATNTFFKPPCVSVYRCGGCCNSEGLQCMNT | |
| | | STSYLSKTLFEITVPLSQGPKPVTISFANHTSCRC | |
| | | MSKLDVYRQVHSIIRR | |
| 733 | >7 minor mature | TEETIKFAAahynteilksidnewrktqcmprevcidvgkefgv | Capital: fragment of |
| | (103-227)- | atntffkppcvsvyrcggccnseglqcmntstsylsktlfeitv | propeptide |
| | VEGFC_His | plsqgpkpvtisfanhtscrcmskldvyrqvhsiirrgshhhhhh | Double underlined: |
| | | | GS linker |
| | | | Bold: his tag |
| 734 | >7 minor mature | TEETIKFAAahynteilksidnewrktqcmprevcidvgkefgv | Capital: fragment of |
| | (103-227)-VEGFC | atntffkppcvsvyrcggccnseglqcmntstsylsktlfeitv | propeptide |
| | | plsqgpkpvtisfanhtscrcmskldvyrqvhsiirr | |
| 735 | >8 minor mature | TEETIKFAAahynteilksidnewrktqcmprevAidvgkefgv | Capital: fragment of |
| | with mutation | atntffkppcvsvyrcggccnseglqcmntstsylsktlfeitv | propeptide |
| | (103-227)- | plsqgpkpvtisfanhtscrcmskldvyrqvhsiirrgshhhhh | Capital, bold A: C |
| | VEGFC_C137A_His | h | to A mutation |
| | | | Double underlined: |
| | | | GS linker |
| | | | Bold: his tag |
| 736 | >8 minor mature | TEETIKFAAahynteilksidnewrktqcmprevAidvgkefgv | Capital: fragment of |
| | with mutation | atntffkppcvsvyrcggccnseglqcmntstsylsktlfeitv | propeptide |
| | (103-227)- | plsqgpkpvtisfanhtscrcmskldvyrqvhsiirr | Capital, bold A: C |
| | VEGFC_C137A | | to A mutation |
| 737 | >9 major mature | ahynteilksidnewrktqcmprevAidvgkefgvatntffkpp | Capital, bold A: C |
| | with mutation | cvsvyrcggccnseglqcmntstsylsktlfeitvplsqgpkpv | to A mutation |
| | (112-227)- | tisfanhtscrcmskldvyrqvhsiirrgshhhhhh | Double underlined: |
| | VEGFC_C137A_His | | GS linker |
| | | | Bold: his tag |
| 738 | >9 major mature | ahynteilksidnewrktqcmprevAidvgkefgvatntffkpp | Capital, bold A: C |
| | with mutation | cvsvyrcggccnseglqcmntstsylsktlfeitvplsqgpkpv | to A mutation |
| | (112-227)- | tisfanhtscrcmskldvyrqvhsiirr | |
| | VEGFC_C137A | | |
| 739 | Propeptide | FESGLDLSDAEPDAGEATAYASKDLEEQLRSVS | |
| | | SVDELMTVLYPEYWKMYKCQLRKGGWQHNRE | |
| | | QANLNSRTEETIKFAA | |
| 740 | Fragment of | TEETIKFAA | |
| | Propeptide | | |
| 741 | VEGF-C consensus | ahynteilksidnewrktqcmprevXidvgkefgvatntffkpp | wherein X is C or A |
| | | cvsvyrcggccnseglqcmntstsylsktlfeitvplsqgpkpv | |
| | | tisfanhtscrcmskldvyrqvhsiirr | |
| 742 | VEGF-C | ahynteilksidnewrktqcmprevcidvgkefgvatntffkpp | |
| | | cvsvyrcggccnseglqcmntstsylsktlfeitvplsqgpkpv | |
| | | tisfanhtscrcmskldvyrqvhsiirr | |
| 743 | VEGF-C with | ahynteilksidnewrktqcmprevAidvgkefgvatntffkpp | Capital, bold A: C |
| | mutation | cvsvyrcggccnseglqcmntstsylsktlfeitvplsqgpkpv | to A mutation |
| | | tisfanhtscrcmskldvyrqvhsiirr | |

In some embodiments, the VEGF-C is present in an effective amount, optionally in an amount of less than or about 1 mg, less than or about 10 mg, greater than or about 10 μg greater than or about 1 μg between about 1 μg and 1 mg, between about 10 μg and 1 mg, between about 1 μg and 10 mg, or between about 10 μg and 10 mg.

In some embodiments, the cell recruitment factor can induce, e.g., promote, migration of immune cells, e.g., T cells. In some embodiments, the cell recruitment factor can increase the expansion or proliferation of a population of immune cells, e.g., T cells. In some embodiments, the cell recruitment factor comprises IL-15 (for example, hetIL-15 (IL15/sIL-15Ra)) or a functional variant thereof. Without wishing to be bound by theory, it is believed that use of a cell recruitment factor, e.g., IL-15 (for example hetIL-15 (IL15/sIL-15Ra)) or a variant thereof, in combination with a cryogel induces immune cell expansion or proliferation, resulting in localized activation as well as promotion and enhancement of migration of immune cells, e.g., T cells, into the cryogel.

In some embodiments, the cell recruitment factor enhances immune cell, e.g., T cell, survival. In some embodiments, the cell recruitment factor comprises IL-7 or a functional variant thereof Without wishing to be bound by theory, it is believed that use of a cell recruitment factor, e.g., IL-7 or functional variant thereof, in combination with a cryogel enhances immune cell, e.g., T cell survival, and proliferation.

In some embodiments, one, two, three, or more cell recruitment factors are to be used in a composition or method described herein. In some embodiments, the cell recruitment factor comprises VEGF-C or a functional variant thereof; IL-15 (for example, hetIL-15 (IL15/sIL-15Ra)) or a functional variant thereof; IL-7 or a functional variant thereof; or a combination thereof. In some embodiments, the cell recruitment factor comprises VEGF-C or a functional variant thereof and IL-15 (for example, hetIL-15 (IL15/sIL-15Ra)) or a functional variant thereof In some embodiments, the cell recruitment factor comprises VEGF-C or a functional variant thereof and IL-7 or a functional variant. In some embodiments, the cell recruitment factor comprises VEGF-C or a functional variant thereof; IL-15 (for example, hetIL-15 (IL15/sIL-15Ra)) or a functional variant thereof; and IL-7 or a functional variant thereof. In some embodiments, the cell recruitment factor comprises IL-15 (for example, hetIL-15 (IL15/sIL-15Ra)) or a functional variant thereof; and IL-7 or a functional variant thereof.

Agent to Promote T Cell Function

In some embodiments, a composition or method described herein utilizes an agent that promotes immune cell, e.g. T cell, function. In some embodiments, the agent to promote T cell, function reduces T cell exhaustion and/or prevents T cell dysfunction.

In some embodiments, the agent to promote T cell function comprises an inhibitor of a Tet2 gene, e.g., a Tet2 inhibitor. While not wishing to be bound by the theory, disruption of a single allele of a Tet gene (e.g., a Tet1, Tet2, or Tet3) leads to decreased total levels of 5-hydroxymethylcytosine in association with enhanced proliferation, regulation of effector cytokine production and degranulation, and thereby increases CAR T cell proliferation and/or function. In some embodiments, the Tet2 inhibitor comprises: (1) a gene editing system targeted to one or more sites within the gene encoding Tet2, or its corresponding regulatory elements; (2) a nucleic acid (e.g., an siRNA or shRNA) that inhibits expression of Tet2; (3) a protein (e.g., a dominant negative, e.g., catalytically inactive) Tet2, or a binding partner of Tet2 (e.g., a dominant negative binding partner of Tet2); (4) a small molecule that inhibits expression and/or function of Tet2; (5) a nucleic acid encoding any of (1)-(3); or (6) any combination of (1)-(5). In some embodiments, the agent to promote T cell function comprises a Tet2 inhibitor as described in, e.g., WO2017/049166, WO2018/175733, and WO2019/210153, the contents of which are hereby incorporated by reference in their entirety.

In some embodiments, the agent to promote T cell function comprises an inhibitor of ZBTB32, e.g., a ZBTB32 inhibitor. Without wishing to be bound by theory, it is believed that in some embodiments, inhibition of ZBTB32 can enhance T cell-mediated anti-tumor response. In certain embodiments, inhalation of ZBTB32 enhances CART cell activity, e.g., cell expansion, cytokine production, persistence, resistance to exhaustion, and anti-tumor activity in vivo. In some embodiments, the ZBTB32 inhibitor comprises: (1) a gene editing system targeting the ZBTB32 gene or one or more components thereof, (2) a nucleic acid encoding one or more components of the gene editing system; or (3) a combination of (1) and (2). In an embodiment, the ZBTB32 inhibitor comprises: (1) a gene editing system targeting the ZBTB32 gene or one or more components thereof. In an embodiment, the ZBTB32 inhibitor comprises (2) a nucleic acid encoding one or more components of the gene editing system. In an embodiment, the ZBTB32 inhibitor comprises a combination of (1) and (2). In some embodiments, the agent to promote T cell function comprises a ZBTB32 inhibitor as described in PCT/US2021/037048, the contents of which are hereby incorporated by reference in their entirety.

Extended Release Agent

In some embodiments, a composition or method described herein utilizes an extended release agent. In some embodiments, the extended release agent can be used to provide extended release of a viral vector (e.g., a lentiviral vector, e.g., a lentiviral vector encoding a CAR), a cell activation agent, or both a viral vector and a cell activation agent. In some embodiments, the extended release agent is formulated for administration by injection. In some embodiments, the extended release agent comprises a particle, e.g., a silica particle, e.g., a mesoporous silica particle.

Surface Modified Mesoporous Silica Particles

In some embodiments, a composition or method described herein utilizes a mesoporous silica particle. Mesoporous silica particles comprise a porous body, for example, with hexagonal close-packed, cylinder-shaped, uniform pores. Mesoporous silica particles can be synthesized by using a rod-like micelle of a surfactant as a template, which is formed in water by dissolving and hydrolyzing a silica source such as alkoxysilane, sodium silicate solution, kanemite, silica fine particle in water or alcohol in the presence of acid or basic catalyst. See, e.g., US Pub. No. 2015-0072009 and Hoffmann et al., Angewandte Chemie International Edition, 45, 3216-3251, 2006. Many kinds of surfactants such as cationic, anionic, and nonionic surfactants have been examined as the surfactant and it has been known that generally, an alkyl trimethylammonium salt of cationic surfactant leads to a mesoporous silica having the greatest specific surface area and a pore volume. See, U.S. Publication No. 2013/0052117 and Katiyar et al. (Journal of Chromatography 1122 (1-2): 13-20).

The mesoporous silica particles may be provided in various forms, e.g., microspheres, irregular particles, rectangular rods, round nanorods. The mesoporous silica particles can have various predetermined shapes, including, e.g., a spheroid shape, an ellipsoid shape, a rod-like shape, or a curved cylindrical shape. In particular embodiments, the compositions and methods recited herein use mesoporous silica rods (MSR). Methods of assembling mesoporous silica to generate microrods are known in the art. See, Wang et al, Journal of Nanoparticle Research, 15: 1501, 2013. In some embodiments, mesoporous silica particles are synthesized by reacting tetraethyl orthosilicate with a template made of micellar rods. The result is a collection of mesoporous silica spheres or rods that are filled with a regular arrangement of pores. The template can then be removed by washing with a solvent adjusted to the proper pH. In this example, after removal of surfactant templates, the mesoporous silica particles are characterized by a uniform, ordered, and connected mesoporosity are prepared with a specific surface area of, for example, about 600 m²/g to about 1200 m²/g, particularly about 800 m²/g to about 1000 m²/g and especially about 850 m²/g to about 950 m²/g. In another embodiment, the mesoporous silica particles may be synthesized using a sol-gel method or a spray drying method. Tetraethyl orthosilicate is also used with an additional polymer monomer (as a template). In yet another embodiment, one or more tetraalkoxy-silanes and one or more (3-cyanopropyl)trialkoxy-silanes may be co-condensed to provide the mesoporous silicate particles as rods. See, US Publication Nos. 2013-0145488, 2012-0264599 and 2012-0256336, the content of which are incorporated by reference in their entireties.

The mesoporous silica particles (MSPs) (e.g., MSRs) may comprise pores, which may be ordered or randomly distributed, of between 2 to 100 nm in diameter, or 2-50 nm in diameter, e.g., pores of between 2-5 nm, 10⁻²⁰ nm, 10⁻³⁰ nm, 10⁻⁴⁰ nm, 20-30 nm, 30-50 nm, 30-40 nm, 40-50 nm. In some embodiments, the microrods comprise pores of approximately 5 nm, 6 nm, 7 nm, 8 nm, 9 nm, 10 nm, 11 nm, 12 nm, or more in diameter. The pore size may be altered depending on the type of application.

In some embodiments, the length of the MSRs is in the micrometer range, ranging from about 5 μm to about 500 μm. In one example, the MSRs comprise a length of 5-50 μm, e.g., 10-μm, 10⁻³⁰ μm, 10⁻⁴⁰ μm, 20-30 μm, 30-50 μm, 30-40 μm, 40-50 μm. In some embodiments, the MSRs comprise length of 50 m to 250 μm, e.g., about 60 μm, 70 μm, 80 μm, 90 μm, 100 am, 120 μm, 150 μm, 180 μm, 200 μm, 225 μm, or more. In some embodiments, the MSRs having a higher aspect ratio, e.g., with rods comprising a length of 50 m to 200 μm, particularly a length of 80 m to 120 μm, especially a length of about 100 m or more, are used. In yet another embodiment, the MSPs (e.g., MSRs) provide a high surface area for attachment and/or binding to target cells, e.g., T-cells. Methods of obtaining high surface area mesoporous silicates are known in the art. See, e.g., U.S. Pat. No. 8,883,308 and US Publication No. 2011-0253643, the entire contents of which are incorporated by reference herein. In some embodiments, the high surface area is due to the fibrous morphology of the nanoparticles, which makes it possible to obtain a high concentration of highly dispersed and easily accessible moieties on the surface. In certain embodiments, the high surface area MSPs (e.g., MSRs) have a surface area of at least about 100 m²/g, at least 150 m²/g, or at least 300 m²/g. In other embodiments, the high surface area MSPs (e.g., MSRs) have a surface area from about 100 m²/g to about 1000 m²/g, including all values or sub-ranges in between, e.g., 50 m²/g, 100 m²/g, 200 m²/g, 300 m²/g, 400 m²/g, 600 m²/g, 800 m²/g, 100-500 m²/g, 100-300 m²/g, 500-800 m²/g or 500-1000 m²/g.

In some embodiments, the mesoporous silica particles may include a surface modification. As used herein, “surface modification” means attaching or appending functional groups on to the surface of the MSPs (e.g., MSRs). In some embodiments, the functional groups are adsorbed or covalently bonded onto the surfaces lining the pores and/or nanochannels or the surface of the MSPs (e.g., MSRs). As used herein, the “functional group” defines a chemical moiety linked to the MSR. In some embodiments, the functional group is a —OH (hydroxyl), amine, carboxylic acid, phosphonate, halide, azide, alkyne, epoxide, sulfhydryl, disulfide, polyethyleneimine, hydrophobic moiety, or salts thereof. In some embodiments, the functional group (i.e. —OH (hydroxyl), amine, carboxylic acid, phosphonate, halide, azide, alkyne, epoxide, sulfhydryl, disulfide, polyethyleneimine, hydrophobic moiety, or salts thereof) may be separated from the silica surface by a linker. In some embodiments, the functional group is covalently bonded to the MSP or MSR surface via a C₁ to C₂₀ alkyl linker. In other embodiments, the functional group is covalently bonded to the MSP or MSR surface via a polyethyleneglycol linker. In particular embodiments, the polyethylene glycol linker has the formula (—O(CH₂—CH₂—)₁₋₂₅. In particular embodiments, the surface modification is a C₁ to C₂₀ alkyl perhaloalkyl or a C₁ to C₂₀ alkyl perfluoroalkyl.

A general structure of surface modifications may be as follows:

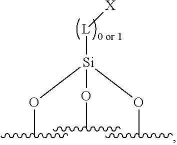

wherein L is a linker, and X is a functional group.

In some embodiments, L may be C₁ to C₂₀ alkyl group or a polyethylene glycol group, and X may be —OH (hydroxyl), primary, secondary, tertiary or quaternary amine, carboxylic acid, phosphonate, halide, azide, alkyne, epoxide, sulfhydryl, disulfide, polyethyleneimine, or hydrophobic moiety, or salts thereof.

As used herein, surface modification having a phosphonate (also known as phosphonate-modified nanoparticles) have at least one phosphonic acid (—P(O)(OH)₂) group or phosphinic acid (—P(O)(OH)R, where R is an C₁ to C₂₀ alkyl group). The phosphonic or phosphinic acid may be charged or uncharged, depending on the pH. At physiological pH, phosphonic acids and phosphinic acids are negatively charged, or anionic. Phosphonate modifications may be prepared, for example, by treating the silica body surface with a phosphonate bearing trialkylsiloxane compound or phosphonate-bearing trihydroxylsilyl compound, such as (trihydroxylsilyl)propyl methylphosphonate.

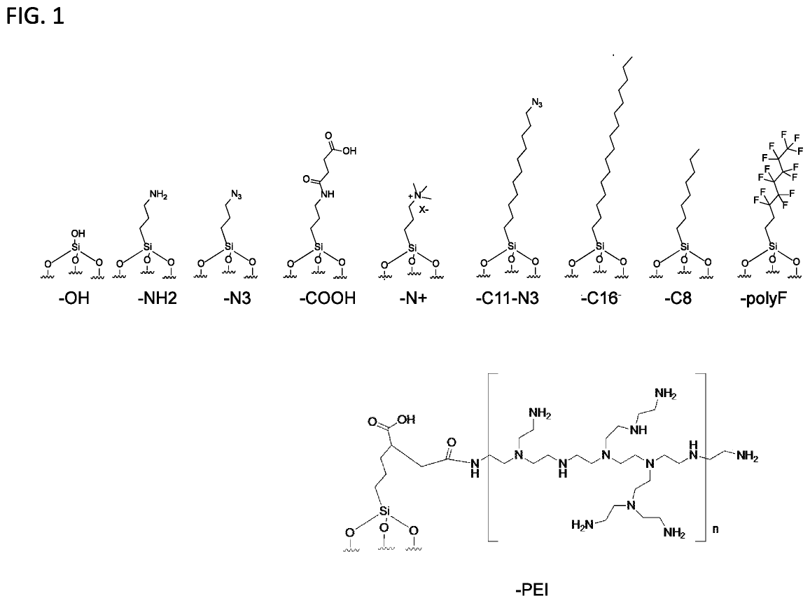

In some embodiments, the mesoporous silica particles (e.g., MSRs) are surface modified with a primary, secondary, tertiary, or quaternary amine. Secondary, tertiary, and quaternary amines may be substituted with C1 to C₂₀ alkyl groups and may be charged. In some embodiments, the amine group may be in the salt form. In some embodiments, the primary, secondary, tertiary, or quaternary amine may be separated from the MSP surface by a linker. In particular embodiments, the mesoporous silica particles are modified with polyethyleneimine. In specific embodiments, the polyethyleneimine is branched or unbranched. In alternate embodiments, the polyethyleneimine group has an average molecular weight in the range of about 1000 to 100,000 Daltons (Da), as measured by gel permeation chromatography (GPC). In some embodiments, the polyethyleneimine group has an average molecular weight of about 1,200 to 15,000 Da, about 1,500 to 12,000 Da, about 2,000 Da, about 3,000 Da, about 4,000 Da, about 5,000 Da, about 6,000 Da, about 7,000 Da, about 8,000 Da, about 9,000 Da, about 10,000 Da, or about 20,000 Da, as measured by gel permeation chromatography (GPC). Structures of various exemplary surface modified mesoporous silica particles are shown in FIG. 1.

The MSPs (e.g., MSRs) recited herein are prepared by methods known to those of skill in the art, as noted herein. Generally, MSPs with surface modification may be prepared by the following method.

In general, any reaction capable of reacting with the silyl hydroxide surface of the MSPs (e.g., MSRs) may be used to covalently modify the surface. For example, the surface of the MSP (e.g. MSR) may be treated with a trialkoxysilyl compound or trihydroxysilyl compound. In some embodiments mesoporous silica particles are suspended in a suitable reaction solvent. In some embodiments, the reaction solvent may be aqueous solvents or buffers of a pH from 0-14. Additional co-mixture of aqueous solutions with 1 or more organic solvents, including but not limited to tetrahydrofuran, 2-methyl tetrahydrofuran, ethyl acetate, toluene, triethylamine, dimethylformamide, dimethylacetamide, dimethylsulfoxide, methanol, ethanol, methylene chloride, or dichloroethane, may be used. In some embodiments, the suspended mesoporous silica particles are reacted with a trialkoxysilyl or trihydroxysilyl reagent having the desired functional group as described herein. Amine modifications may be prepared, for example, by treating the MSPs with an amine bearing trialkoxysilane compound, such as aminopropyltriethoxysilane, 3-(2-aminoethylamino)propyl-trimethoxysilane, or 3-trimethoxysilylpropyl ethylenediamine. In certain embodiments, the trialkoxysilyl is a trimethoxysilyl or triethoxysilyl group. In alternate embodiments, the trialkoxysilyl reagent is a trialkoxy alkylamine. In some embodiments, the trialkoxy alkylamine includes a primary, secondary, tertiary, or quaternary amine.

In certain embodiments, the trialkoxysilyl reagent includes a polyethyleneimine group. In specific embodiments, the polyethyleneimine is branched or unbranched. In alternate embodiments, the polyethyleneimine group has an average molecular weight of about 1000 to 20,000 Da, about 1,200 to 15,000 Da, about 1,500 to 12,000 Da, about 2,000 Da, about 3,000 Da, about 4,000 Da, about 5,000 Da, about 6,000 Da, about 7,000 Da, about 8,000 Da, about 9,000 Da, or about 10,000 Da, as measured by gel permeation chromatography (GPC). In some embodiments, the trialkoxysilyl reagent includes an C₁₋₂₀ alkylazide group. In certain embodiments, the trialkoxysilyl reagent includes an C₁₋₂₀ alkylcarboxylic acid group. In other embodiments, the trialkoxysilyl reagent includes a C₁₋₂₀ alkyl group.

A sulfhydryl modification on a MSP (e.g., MSR) may be prepared, for example, by treating the MSP with a sulfhydryl bearing trialkoxysilane compound, such as 3-mercaptopropyltriethoxysilane.

A disulfide modification on a MSP (e.g., MSR) may be prepared, for example, by treating the surface of the nanoparticle with a disulfide bearing trialkoxysilane compound, or by treating a sulfhydryl modified surface with 2,2′-dithiodipyridine or other disulfide. MSP (e.g., MSR) surface modifications to include a carboxylic acid group may be prepared, for example, by treating the surface with a carboxylic acid bearing trialkoxysilane compound, or by treating the MSP with a trialkoxysilane compound bearing a functional group that may be converted chemically into a carboxylic acid. For example, the MSP may be treated with 3-cyanopropyltriethoxysilane, followed by hydrolysis with sulfuric acid. MSP (e.g., MSR) surface modifications to include an epoxide will have at least one epoxide may be prepared, for example, by treating the MSP with an epoxide bearing trialkoxysilane compound, such as glycidoxypropyltriethoxysilane.

Surface modifications having a hydrophobic moiety will have at least one moiety intended to reduce the solubility in water, or increase the solubility in organic solvents. Examples of hydrophobic moieties include long chain alkyl groups (e.g., C₈-C₂₀ alkyl groups), fatty acid esters (e.g., C₁-C₂₂ alkyl acid esters), and aromatic rings having C₆-C₁₀ carbon atoms. In some embodiments, the reaction of the MSPs (e.g., MSRs) with the trialkoxysilyl reagent is carried out at ambient or room temperature. In other embodiments, the reaction is carried out at elevated temperatures. In further embodiments, the temperature of the reaction is from about 40° C. to about 120° C., about 50° C. to about 100° C., about 60° C. to about 80° C., about 70° C. to about 80° C., or about 50° C., about 55° C., about 60° C., about 65° C., about 70° C., about 75° C., about 80° C., about 85° C., about 90° C., about 95° C., or about 100° C.

Viral Vectors

In some embodiments, the compositions described herein can include an extended release agent, e.g., a mesoporous silica particle as described herein and a viral vector.

The viral vector can be any viral vector. Viral vector technology is well known in the art and is described, for example, in Sambrook et al., 2012, MOLECULAR CLONING: A LABORATORY MANUAL, volumes 1-4, Cold Spring Harbor Press, NY), and in other virology and molecular biology manuals. By way of example, the viral vector can be an adenovirus, a lentivirus, a retrovirus, an adeno-associated virus, or a herpesvirus. In some embodiments, the viral vector is a lentivirus vector or an adenovirus vector.

Vectors derived from retroviruses such as the lentivirus are suitable tools to achieve long-term gene transfer since they allow long-term, stable integration of a transgene and its propagation in daughter cells. Lentiviral vectors have the added advantage over vectors derived from onco-retroviruses such as murine leukemia viruses in that they can transduce non-proliferating cells, such as hepatocytes. They also have the added advantage of low immunogenicity. A retroviral vector may also be, e.g., a gammaretroviral vector. A gammaretroviral vector may include, e.g., a promoter, a packaging signal (W), a primer binding site (PBS), one or more (e.g., two) long terminal repeats (LTR), and a transgene of interest, e.g., a gene encoding a CAR. A gammaretroviral vector may lack viral structural gens such as gag, pol, and env. Exemplary gammaretroviral vectors include Murine Leukemia Virus (MLV), Spleen-Focus Forming Virus (SFFV), and Myeloproliferative Sarcoma Virus (MPSV), and vectors derived therefrom. Other gammaretroviral vectors are described, e.g., in Tobias Maetzig et al., “Gammaretroviral Vectors: Biology, Technology and Application” Viruses. 2011 June; 3(6): 677-713.

In another embodiment, the vector comprising the nucleic acid encoding the desired CAR of the invention is an adenoviral vector (A5/35). In another embodiment, the expression of nucleic acids encoding CARs can be accomplished using of transposons such as sleeping beauty, CRISPR, CAS9, and zinc finger nucleases. See June et al. 2009 Nature Reviews Immunology 9.10: 704-716, is incorporated herein by reference.

In some embodiments, the viral vector comprises an expression vector comprising a recombinant polynucleotide comprising an expression control sequence operatively linked to a nucleotide sequence to be expressed. In some embodiments, the nucleotide sequence expresses a chimeric antigen receptor (CAR), engineered TCR, cytokines, chemokines, shRNA to block an inhibitory molecule, or mRNA to induce expression of protein. In some embodiments, the protein is a CAR that comprises an antigen binding domain, a transmembrane domain, a costimulatory signaling region, and a signaling domain. In some embodiments, the signaling domain is a CD3 zeta signaling domain.

In some embodiments, the nucleotide sequence in the viral vector express a peptide engineered to target a tumor antigen. In some embodiments, the peptide targets a tumor antigen selected from the group consisting of: TSHR, CD19, CD123, CD22, CD30, CD171, CS-1, CLL-1, CD33, EGFRvIII, GD2, GD3, BCMA, Tn Ag, PSMA, ROR1, FLT3, FAP, TAG72, CD38, CD44v6, CEA, EPCAM, B7H3, KIT, IL-13Ra2, Mesothelin, IL-11Ra, PSCA, PRSS21, VEGFR2, LewisY, CD24, PDGFR-beta, SSEA-4, CD20, Folate receptor alpha, ERBB2 (Her2/neu), MUC1, EGFR, NCAM, Prostase, PAP, ELF2M, Ephrin B2, IGF-I receptor, CAIX, LMP2, gp100, bcr-abl, tyrosinase, EphA2, Fucosyl GM1, sLe, GM3, TGS5, HMWMAA, o-acetyl-GD2, Folate receptor beta, TEM1/CD248, TEM7R, CLDN6, GPRC5D, CXORF61, CD97, CD179a, ALK, Polysialic acid, PLAC1, GloboH, NY-BR-1, UPK2, HAVCR1, ADRB3, PANX3, GPR20, LY6K, OR51E2, TARP, WT1, NY-ESO-1, LAGE-1a, MAGE-A1, legumain, HPV E6,E7, MAGE A1, ETV6-AML, sperm protein 17, XAGE1, Tie 2, MAD-CT-1, MAD-CT-2, Fos-related antigen 1, p53, p53 mutant, prostein, survivin and telomerase, PCTA-1/Galectin 8, MelanA/MART1, Ras mutant, hTERT, sarcoma translocation breakpoints, ML-IAP, ERG (TMPRSS2 ETS fusion gene), NA17, PAX3, Androgen receptor, Cyclin B1, MYCN, RhoC, TRP-2, CYP1B1, BORIS, SART3, PAX5, OY-TES1, LCK, AKAP-4, SSX2, RAGE-1, human telomerase reverse transcriptase, RU1, RU2, intestinal carboxyl esterase, mut hsp70-2, CD79a, CD79b, CD72, LAIR1, FCAR, LILRA2, CD300LF, CLEC12A, BST2, EMR2, LY75, GPC3, FCRL5, IGLL1, and any combination thereof. In some embodiments, the peptide is a chimeric antigen receptor (CAR) or an engineered TCR. Such peptides are described in greater detail herein below in the section entitled General Description of Chimeric Antigen Receptor Technology.

In some embodiments, the nucleotide sequence in the vector expresses a protein engineered to target a tumor antigen. In some embodiments, the tumor antigen is chosen from one or more of: CD19; CD123; CD22; CD30; CD171; CS-1 (also referred to as CD2 subset 1, CRACC, SLAMF7, CD319, and 19A24); C-type lectin-like molecule-1 (CLL-1 or CLECL1); CD33; epidermal growth factor receptor variant III (EGFRvIII); ganglioside G2 (GD2); ganglioside GD3 (aNeu5Ac(2-8)aNeu5Ac(2-3)bDGalp(1-4)bDGlcp(1-1)Cer); TNF receptor family member B cell maturation (BCMA); Tn antigen ((Tn Ag) or (GalNAca-Ser/Thr)); prostate-specific membrane antigen (PSMA); Receptor tyrosine kinase-like orphan receptor 1 (ROR1); Fms-Like Tyrosine Kinase 3 (FLT3); Tumor-associated glycoprotein 72 (TAG72); CD38; CD44v6; Carcinoembryonic antigen (CEA); Epithelial cell adhesion molecule (EPCAM); B7H3 (CD276); KIT (CD 117); Interleukin-13 receptor subunit alpha-2 (IL-13Ra2 or CD213A2); Mesothelin; Interleukin 11 receptor alpha (IL-11Ra); prostate stem cell antigen (PSCA); Protease Serine 21 (Testisin or PRSS21); vascular endothelial growth factor receptor 2 (VEGFR2); Lewis(Y) antigen; CD24; Platelet-derived growth factor receptor beta (PDGFR-beta); Stage-specific embryonic antigen-4 (SSEA-4); CD20; Folate receptor alpha; Receptor tyrosine-protein kinase ERBB2 (Her2/neu); Mucin 1, cell surface associated (MUC1); epidermal growth factor receptor (EGFR); neural cell adhesion molecule (NCAM); Prostase; prostatic acid phosphatase (PAP); elongation factor 2 mutated (ELF2M); Ephrin B2; fibroblast activation protein alpha (FAP); insulin-like growth factor 1 receptor (IGF-I receptor), carbonic anhydrase IX (CAIX); Proteasome (Prosome, Macropain) Subunit, Beta Type, 9 (LMP2); glycoprotein 100 (gp100); oncogene fusion protein consisting of breakpoint cluster region (BCR) and Abelson murine leukemia viral oncogene homolog 1 (Abl) (bcr-abl); tyrosinase; ephrin type-A receptor 2 (EphA2); Fucosyl GM1; sialyl Lewis adhesion molecule (sLe); ganglioside GM3 (aNeu5Ac(2-3)bDGalp(1-4)bDGlcp(1-1)Cer); transglutaminase 5 (TGS5); high molecular weight-melanoma-associated antigen (HMWMAA); o-acetyl-GD2 ganglioside (OAcGD2); Folate receptor beta; tumor endothelial marker 1 (TEM1/CD248); tumor endothelial marker 7-related (TEM7R); claudin 6 (CLDN6); thyroid stimulating hormone receptor (TSHR); G protein-coupled receptor class C group 5, member D (GPRC5D); chromosome X open reading frame 61 (CXORF61); CD97; CD179a; anaplastic lymphoma kinase (ALK); Polysialic acid; placenta-specific 1 (PLAC1); hexasaccharide portion of globoH glycoceramide (GloboH); mammary gland differentiation antigen (NY-BR-1); uroplakin 2 (UPK2); Hepatitis A virus cellular receptor 1 (HAVCR1); adrenoceptor beta 3 (ADRB3); pannexin 3 (PANX3); G protein-coupled receptor 20 (GPR20); lymphocyte antigen 6 complex, locus K 9 (LY6K); Olfactory receptor 51E2 (OR51E2); TCR Gamma Alternate Reading Frame Protein (TARP); Wilms tumor protein (WT1); Cancer/testis antigen 1 (NY-ESO-1); Cancer/testis antigen 2 (LAGE-1a); Melanoma-associated antigen 1 (MAGE-A1); ETS translocation-variant gene 6, located on chromosome 12p (ETV6-AML); sperm protein 17 (SPA17); X Antigen Family, Member 1A (XAGE1); angiopoietin-binding cell surface receptor 2 (Tie 2); melanoma cancer testis antigen-1 (MAD-CT-1); melanoma cancer testis antigen-2 (MAD-CT-2); Fos-related antigen 1; tumor protein p53 (p53); p53 mutant; prostein; surviving; telomerase; prostate carcinoma tumor antigen-1 (PCTA-1 or Galectin 8), melanoma antigen recognized by T cells 1 (MelanA or MART1); Rat sarcoma (Ras) mutant; human Telomerase reverse transcriptase (hTERT); sarcoma translocation breakpoints; melanoma inhibitor of apoptosis (ML-IAP); ERG (transmembrane protease, serine 2 (TMPRSS2) ETS fusion gene); N-Acetyl glucosaminyl-transferase V (NA17); paired box protein Pax-3 (PAX3); Androgen receptor; Cyclin B1; v-myc avian myelocytomatosis viral oncogene neuroblastoma derived homolog (MYCN); Ras Homolog Family Member C (RhoC); Tyrosinase-related protein 2 (TRP-2); Cytochrome P450 1B1 (CYP1B1); CCCTC-Binding Factor (Zinc Finger Protein)-Like (BORIS or Brother of the Regulator of Imprinted Sites), Squamous Cell Carcinoma Antigen Recognized By T Cells 3 (SART3); Paired box protein Pax-5 (PAX5); proacrosin binding protein sp32 (OY-TES1); lymphocyte-specific protein tyrosine kinase (LCK); A kinase anchor protein 4 (AKAP-4); synovial sarcoma, X breakpoint 2 (SSX2); Receptor for Advanced Glycation End products (RAGE-1); renal ubiquitous 1 (RU1); renal ubiquitous 2 (RU2); legumain; human papilloma virus E6 (HPV E6); human papilloma virus E7 (HPV E7); intestinal carboxyl esterase; heat shock protein 70-2 mutated (mut hsp70-2); CD79a; CD79b; CD72; Leukocyte-associated immunoglobulin-like receptor 1 (LAIR1); Fc fragment of IgA receptor (FCAR or CD89); Leukocyte immunoglobulin-like receptor subfamily A member 2 (LILRA2); CD300 molecule-like family member f (CD300LF); C-type lectin domain family 12 member A (CLEC12A); bone marrow stromal cell antigen 2 (BST2); EGF-like module-containing mucin-like hormone receptor-like 2 (EMR2); lymphocyte antigen 75 (LY75); Glypican-3 (GPC3); Fc receptor-like 5 (FCRL5); and immunoglobulin lambda-like polypeptide 1 (IGLL1).

A CAR described herein can comprise an antigen binding domain (e.g., antibody or antibody fragment, TCR or TCR fragment) that binds to a tumor-supporting antigen (e.g., a tumor-supporting antigen as described herein). In some embodiments, the tumor-supporting antigen is an antigen present on a stromal cell or a myeloid-derived suppressor cell (MDSC). Stromal cells can secrete growth factors to promote cell division in the microenvironment. MDSC cells can inhibit T cell proliferation and activation. Without wishing to be bound by theory, in some embodiments, the CAR-expressing cells destroy the tumor-supporting cells, thereby indirectly inhibiting tumor growth or survival.

In some embodiments, the stromal cell antigen is chosen from one or more of: bone marrow stromal cell antigen 2 (BST2), fibroblast activation protein (FAP) and tenascin. In some embodiments, the FAP-specific antibody is, competes for binding with, or has the same CDRs as, sibrotuzumab. In embodiments, the MDSC antigen is chosen from one or more of: CD33, CD11b, C14, CD15, and CD66b. Accordingly, in some embodiments, the tumor-supporting antigen is chosen from one or more of: bone marrow stromal cell antigen 2 (BST2), fibroblast activation protein (FAP) or tenascin, CD33, CD11b, C14, CD15, and CD66b.

In some embodiments, the antigen binding domain of the encoded CAR molecule comprises an antibody, an antibody fragment, an scFv, a Fv, a Fab, a (Fab′)2, a single domain antibody (SDAB), a VH or VL domain, a camelid VHH domain or a bi-functional (e.g. bi-specific) hybrid antibody (e.g., Lanzavecchia et al., Eur. J. Immunol. 17, 105 (1987)).

In some instances, scFvs can be prepared according to method known in the art (see, for example, Bird et al., (1988) Science 242:423-426 and Huston et al., (1988) Proc. Natl. Acad. Sci. USA 85:5879-5883). ScFv molecules can be produced by linking VH and VL regions together using flexible polypeptide linkers. The scFv molecules comprise a linker (e.g., a Ser-Gly linker) with an optimized length and/or amino acid composition. The linker length can greatly affect how the variable regions of a scFv fold and interact. In fact, if a short polypeptide linker is employed (e.g., between 5-10 amino acids) intrachain folding is prevented. Interchain folding is also required to bring the two variable regions together to form a functional epitope binding site. For examples of linker orientation and size see, e.g., Hollinger et al. 1993 Proc Natl Acad. Sci. U.S.A. 90:6444-6448, U.S. Patent Application Publication Nos. 2005/0100543, 2005/0175606, 2007/0014794, and PCT publication Nos. WO2006/020258 and WO2007/024715, is incorporated herein by reference.

An scFv can comprise a linker of at least 1, 2, 3, 4, 5, 6, 7, 8, 9, 10, 11, 12, 13, 14, 15, 16, 17, 18, 19, 20, 25, 30, 35, 40, 45, 50, or more amino acid residues between its VL and VH regions. The linker sequence may comprise any naturally occurring amino acid. In some embodiments, the linker sequence comprises amino acids glycine and serine. In another embodiment, the linker sequence comprises sets of glycine and serine repeats such as (Gly₄Ser)n, where n is a positive integer equal to or greater than 1 (SEQ ID NO: 22). In some embodiments, the linker can be (Gly₄Ser)₄ (SEQ ID NO:29) or (Gly₄Ser)₃(SEQ ID NO:30). Variation in the linker length may retain or enhance activity, giving rise to superior efficacy in activity studies. In another aspect, the antigen binding domain is a T cell receptor (“TCR”), or a fragment thereof, for example, a single chain TCR (scTCR). Methods to make such TCRs are known in the art. See, e.g., Willemsen R A et al, Gene Therapy 7: 1369-1377 (2000); Zhang T et al, Cancer Gene Ther 11: 487-496 (2004); Aggen et al, Gene Ther. 19(4):365-74 (2012) (references are incorporated herein by its entirety). For example, scTCR can be engineered that contains the Vα and Vβ genes from a T cell clone linked by a linker (e.g., a flexible peptide). This approach is very useful to cancer associated target that itself is intracellular, however, a fragment of such antigen (peptide) is presented on the surface of the cancer cells by MHC.

In certain embodiments, the encoded antigen binding domain has a binding affinity KD of 10⁻⁴ M to 10⁻⁸ M.

In some embodiments, the encoded CAR molecule comprises an antigen binding domain that has a binding affinity KD of 10⁻⁴ M to 10⁻⁸ M, e.g., 10⁻⁵ M to 10⁻⁷ M, e.g., 10⁻⁶ M or 10⁻⁷ M, for the target antigen. In some embodiments, the antigen binding domain has a binding affinity that is at least five-fold, 10-fold, 20-fold, 30-fold, 50-fold, 100-fold or 1,000-fold less than a reference antibody, e.g., an antibody described herein. In some embodiments, the encoded antigen binding domain has a binding affinity at least 5-fold less than a reference antibody (e.g., an antibody from which the antigen binding domain is derived). In some aspects such antibody fragments are functional in that they provide a biological response that can include, but is not limited to, activation of an immune response, inhibition of signal-transduction origination from its target antigen, inhibition of kinase activity, and the like, as will be understood by a skilled artisan.

In some aspects, the antigen binding domain of the CAR is a scFv antibody fragment that is humanized compared to the murine sequence of the scFv from which it is derived.

In some aspects, the antigen binding domain of a CAR of the invention (e.g., a scFv) is encoded by a nucleic acid molecule whose sequence has been codon optimized for expression in a mammalian cell. In some aspects, entire CAR construct of the invention is encoded by a nucleic acid molecule whose entire sequence has been codon optimized for expression in a mammalian cell. Codon optimization refers to the discovery that the frequency of occurrence of synonymous codons (i.e., codons that code for the same amino acid) in coding DNA is biased in different species. Such codon degeneracy allows an identical polypeptide to be encoded by a variety of nucleotide sequences. A variety of codon optimization methods is known in the art, and include, e.g., methods disclosed in at least U.S. Pat. Nos. 5,786,464 and 6,114,148.

In some embodiments involving immune effector cells engineered to express a CAR molecule, e.g., as described herein, it is understood that the treatment method may further include any of the steps, aspects or features described below in the section relating to Chimeric Antigen Receptors.

The cells are preferably immune effector cells. In some embodiments, the cells are T cells. In some embodiments, the cells are NK cells. In embodiments, the invention relates to a population of cells of the invention, e.g., a population of immune effector cells of the invention. In embodiments, the population of cells of the invention comprises cells of the type indicated, and may comprise other types (e.g., a population of immune effector cells, e.g., T cells, engineered to express a CAR molecule, e.g., as described herein, may include T cells engineered to express a CAR molecule as well as T cells (or other cell types) that have not been engineered to express a CAR molecule). In embodiments, the population of cells used in the methods of the invention consists essentially of cells of the type indicated. In embodiments, the population of cells of the invention is substantially free of other cell types. In embodiments, the population of cells of the invention consists of the indicated cell type.

In any of the foregoing aspects and embodiments, the cells and/or population of cells are or include immune effector cells, e.g., the population of immune effector cells includes, e.g., consists of, T cells or NK cells. In embodiments the cells are T cells, e.g., CD8+ T cells, CD4+T cells, or a combination thereof. In embodiments the cells are NK cells.

In embodiments the cells are human cells. In embodiments, the cells are autologous, e.g., to the subject to be administered the cells. In embodiments, the cells are allogeneic, e.g., to the subject to be administered the cells.

In general, in the methods described herein, the compositions described herein may be administered in therapeutically effective amounts as described above via any of the usual and acceptable modes known in the art, either singly or in combination with one or more therapeutic agents. In particular embodiments, the compositions are administered by injection. In still particular embodiments, for in vivo administration, the compositions are administered subcutaneously to a subject in need thereof. In other embodiments, the compositions may be administered in the form of an implant at the desired site of action. The site of action may be determined by a person of skill in the art in accordance with the needs of the subject.

### CAR Targets

Described herein are viral vectors to transduce immune effector cells (e.g., T cells, NK cells) that are engineered to contain one or more CARs that direct the immune effector cells to undesired cells (e.g., cancer cells). This is achieved through an antigen binding domain on the CAR that is specific for a cancer associated antigen. There are two classes of cancer associated antigens (tumor antigens) that can be targeted by the CARs of the instant invention: (1) cancer associated antigens that are expressed on the surface of cancer cells; and (2) cancer associated antigens that itself is intracellular, however, a fragment of such antigen (peptide) is presented on the surface of the cancer cells by MHC (major histocompatibility complex).

In some embodiments, the tumor antigen is chosen from one or more of: CD19; CD123; CD22; CD30; CD171; CS-1 (also referred to as CD2 subset 1, CRACC, SLAMF7, CD319, and 19A24); C-type lectin-like molecule-1 (CLL-1 or CLECL1); CD33; epidermal growth factor receptor variant III (EGFRvIII); ganglioside G2 (GD2); ganglioside GD3 (aNeu5Ac(2-8)aNeu5Ac(2-3)bDGalp(1-4)bDGlcp(1-1)Cer); TNF receptor family member B cell maturation (BCMA); Tn antigen ((Tn Ag) or (GalNAca-Ser/Thr)); prostate-specific membrane antigen (PSMA); Receptor tyrosine kinase-like orphan receptor 1 (ROR1); Fms-Like Tyrosine Kinase 3 (FLT3); Tumor-associated glycoprotein 72 (TAG72); CD38; CD44v6; Carcinoembryonic antigen (CEA); Epithelial cell adhesion molecule (EPCAM); B7H3 (CD276); KIT (CD 117); Interleukin-13 receptor subunit alpha-2 (IL-13Ra2 or CD213A2); Mesothelin; Interleukin 11 receptor alpha (IL-11Ra); prostate stem cell antigen (PSCA); Protease Serine 21 (Testisin or PRSS21); vascular endothelial growth factor receptor 2 (VEGFR2); Lewis(Y) antigen; CD24; Platelet-derived growth factor receptor beta (PDGFR-beta); Stage-specific embryonic antigen-4 (SSEA-4); CD20; Folate receptor alpha; Receptor tyrosine-protein kinase ERBB2 (Her2/neu); Mucin 1, cell surface associated (MUC1); epidermal growth factor receptor (EGFR); neural cell adhesion molecule (NCAM); Prostase; prostatic acid phosphatase (PAP); elongation factor 2 mutated (ELF2M); Ephrin B2; fibroblast activation protein alpha (FAP); insulin-like growth factor 1 receptor (IGF-I receptor), carbonic anhydrase IX (CAIX); Proteasome (Prosome, Macropain) Subunit, Beta Type, 9 (LMP2); glycoprotein 100 (gp100); oncogene fusion protein consisting of breakpoint cluster region (BCR) and Abelson murine leukemia viral oncogene homolog 1 (Abl) (bcr-abl); tyrosinase; ephrin type-A receptor 2 (EphA2); Fucosyl GM1; sialyl Lewis adhesion molecule (sLe); ganglioside GM3 (aNeu5Ac(2-3)bDGalp(1-4)bDGlcp(1-1)Cer); transglutaminase 5 (TGS5); high molecular weight-melanoma-associated antigen (HMWMAA); o-acetyl-GD2 ganglioside (OAcGD2); Folate receptor beta; tumor endothelial marker 1 (TEM1/CD248); tumor endothelial marker 7-related (TEM7R); claudin 6 (CLDN6); thyroid stimulating hormone receptor (TSHR); G protein-coupled receptor class C group 5, member D (GPRC5D); chromosome X open reading frame 61 (CXORF61); CD97; CD179a; anaplastic lymphoma kinase (ALK); Polysialic acid; placenta-specific 1 (PLAC1); hexasaccharide portion of globoH glycoceramide (GloboH); mammary gland differentiation antigen (NY-BR-1); uroplakin 2 (UPK2); Hepatitis A virus cellular receptor 1 (HAVCR1); adrenoceptor beta 3 (ADRB3); pannexin 3 (PANX3); G protein-coupled receptor 20 (GPR20); lymphocyte antigen 6 complex, locus K 9 (LY6K); Olfactory receptor 51E2 (OR51E2); TCR Gamma Alternate Reading Frame Protein (TARP); Wilms tumor protein (WT1); Cancer/testis antigen 1 (NY-ESO-1); Cancer/testis antigen 2 (LAGE-1a); Melanoma-associated antigen 1 (MAGE-A1); ETS translocation-variant gene 6, located on chromosome 12p (ETV6-AML); sperm protein 17 (SPA17); X Antigen Family, Member 1A (XAGE1); angiopoietin-binding cell surface receptor 2 (Tie 2); melanoma cancer testis antigen-1 (MAD-CT-1); melanoma cancer testis antigen-2 (MAD-CT-2); Fos-related antigen 1; tumor protein p53 (p53); p53 mutant; prostein; surviving; telomerase; prostate carcinoma tumor antigen-1 (PCTA-1 or Galectin 8), melanoma antigen recognized by T cells 1 (MelanA or MART1); Rat sarcoma (Ras) mutant; human Telomerase reverse transcriptase (hTERT); sarcoma translocation breakpoints; melanoma inhibitor of apoptosis (ML-IAP); ERG (transmembrane protease, serine 2 (TMPRSS2) ETS fusion gene); N-Acetyl glucosaminyl-transferase V (NA17); paired box protein Pax-3 (PAX3); Androgen receptor; Cyclin B1; v-myc avian myelocytomatosis viral oncogene neuroblastoma derived homolog (MYCN); Ras Homolog Family Member C (RhoC); Tyrosinase-related protein 2 (TRP-2); Cytochrome P450 1B1 (CYP1B1); CCCTC-Binding Factor (Zinc Finger Protein)-Like (BORIS or Brother of the Regulator of Imprinted Sites), Squamous Cell Carcinoma Antigen Recognized By T Cells 3 (SART3); Paired box protein Pax-5 (PAX5); proacrosin binding protein sp32 (OY-TES1); lymphocyte-specific protein tyrosine kinase (LCK); A kinase anchor protein 4 (AKAP-4); synovial sarcoma, X breakpoint 2 (SSX2); Receptor for Advanced Glycation End products (RAGE-1); renal ubiquitous 1 (RU1); renal ubiquitous 2 (RU2); legumain; human papilloma virus E6 (HPV E6); human papilloma virus E7 (HPV E7); intestinal carboxyl esterase; heat shock protein 70-2 mutated (mut hsp70-2); CD79a; CD79b; CD72; Leukocyte-associated immunoglobulin-like receptor 1 (LAIR1); Fc fragment of IgA receptor (FCAR or CD89); Leukocyte immunoglobulin-like receptor subfamily A member 2 (LILRA2); CD300 molecule-like family member f (CD300LF); C-type lectin domain family 12 member A (CLEC12A); bone marrow stromal cell antigen 2 (BST2); EGF-like module-containing mucin-like hormone receptor-like 2 (EMR2); lymphocyte antigen 75 (LY75); Glypican-3 (GPC3); Fc receptor-like 5 (FCRL5); and immunoglobulin lambda-like polypeptide 1 (IGLL1).

CD19

An non-limiting exemplary tumor antigen is CD19. CARs that bind to CD19 are known in the art. For example, those disclosed in WO2012/079000 and WO2014/153270. Any known CD19 CAR, for example, the CD19 antigen binding domain of any known CD19 CAR, in the art can be used in accordance with the present disclosure. For example, LG-740; CD19 CAR described in the U.S. Pat. Nos. 8,399,645; 7,446,190; Xu et al., Leuk Lymphoma. 2013 54(2):255-260(2012); Cruz et al., Blood 122(17):2965-2973 (2013); Brentjens et al., Blood, 118(18):4817-4828 (2011); Kochenderfer et al., Blood 116(20):4099-102 (2010); Kochenderfer et al., Blood 122 (25):4129-39(2013); and 16th Annu Meet Am Soc Gen Cell Ther (ASGCT) (May 15-18, Salt Lake City) 2013, Abst 10.

Non-limiting exemplary CD19 CARs include CD19 CARs described herein or an anti-CD19 CAR described in Xu et al. Blood 123.24(2014):3750-9; Kochenderfer et al. Blood 122.25(2013):4129-39, Cruz et al. Blood 122.17(2013):2965-73, NCT00586391, NCT01087294, NCT02456350, NCT00840853, NCT02659943, NCT02650999, NCT02640209, NCT01747486, NCT02546739, NCT02656147, NCT02772198, NCT00709033, NCT02081937, NCT00924326, NCT02735083, NCT02794246, NCT02746952, NCT01593696, NCT02134262, NCT01853631, NCT02443831, NCT02277522, NCT02348216, NCT02614066, NCT02030834, NCT02624258, NCT02625480, NCT02030847, NCT02644655, NCT02349698, NCT02813837, NCT02050347, NCT01683279, NCT02529813, NCT02537977, NCT02799550, NCT02672501, NCT02819583, NCT02028455, NCT01840566, NCT01318317, NCT01864889, NCT02706405, NCT01475058, NCT01430390, NCT02146924, NCT02051257, NCT02431988, NCT01815749, NCT02153580, NCT01865617, NCT02208362, NCT02685670, NCT02535364, NCT02631044, NCT02728882, NCT02735291, NCT01860937, NCT02822326, NCT02737085, NCT02465983, NCT02132624, NCT02782351, NCT01493453, NCT02652910, NCT02247609, NCT01029366, NCT01626495, NCT02721407, NCT01044069, NCT00422383, NCT01680991, NCT02794961, or NCT02456207, each of which is incorporated herein by reference in its entirety.

In some embodiments, the CD19 CAR comprises the fusion polypeptide sequence provided as SEQ ID NO: 12 in WO2012/079000, which provides an scFv fragment of murine origin that specifically binds to human CD19.
In some embodiments, the CD19 CAR comprises an amino acid sequence provided as SEQ ID NO: 12 in WO2012/079000.

In some embodiments, the CD19 CAR comprises the amino acid sequence: digmtqttsslsaslgdrvtiscrasqdiskylnwyqqkpdgtvklliyhtsrlhsgvpsrfsgsgsgtdysltisnleqediatyfcqqgntl pytfgggtkleitggggsggggsggggsevklgesgpglvapsqslsvtctvsgvslpdygvswirqpprkglewlgviwgsettyyn salksrltiikdnsksqvflkmnslqtddtaiyycakhyyyggsyamdywgqgtsvtvsstttpaprpptpaptiasqplslrpeacrpa aggavhtrgldfacdiyiwaplagtcgvlllslvitlyckrgrkkllyifkqpfmrpvqttgeedgcscrfpeeeeggcelrvkfsrsadap aykqgqnglynelnlgrreeydvldkrrgrdpemggkprrknpqeglynelqkdkmaeayseigmkgerrrgkghdglyqglstat kdtydalhmqalppr (SEQ ID NO: 757), or a sequence substantially homologous thereto.

In some embodiments, the CD19 CAR comprises the amino acid sequence:

(SEQ ID NO: 758)
eivmtqspatlslspgeratlscrasqdiskylnwyqqkpgqaprlliyh
tsrlhsgiparfsgsgsgtdytltisslqpedfavyfcqqgntlpytfgq
gtkleikggggsggggsggggsqvqlqesgpglvkpsetlsltctvsgvs
lpdygvswirqppgkglewigviwgsettyyqsslksrvtiskdnsknqv
slklssvtaadtavyycakhyyyggsyamdywgqgtlvtvss

In some embodiments, the CD19 CAR is a humanized CD19 CAR comprising the amino acid sequence:

(SEQ ID NO: 759)
eivmtqspatlslspgeratlscrasqdiskylnwyqqkpgqaprlliyh
tsrlhsgiparfsgsgsgtdytltisslqpedfavyfcqqgntlpytfgq
gtkleikggggsggggsggggsqvqlqesgpglvkpsetlsltctvsgvs
lpdygvswirqppgkglewigviwgsettyyqsslksrvtiskdnsknqv
slklssvtaadtavyycakhyyyggsyamdywgqgtlvtvsstttpaprp
ptpaptiasqplslrpeacrpaaggavhtrgldfacdiyiwaplagtcgv
lllslvitlyckrgrkkllyifkqpfmrpvqttqeedgcscrfpeeeegg
celrvkfsrsadapaykqgqnqlynelnlgrreeydvldkrrgrdpemgg
kprrknpqeglynelqkdkmaeayseigmkgerrrgkghdglyqglstat
kdtydalhmqalppr

In some embodiments, CD19 CARs comprise a sequence, for example, a CDR, VH, VL, scFv, or full-CAR sequence, disclosed in Table 1 below, or a sequence having at least 80%, 85%, 90%, 95%, or 99% identity thereto.

TABLE 1
Amino acid sequences of exemplary anti-CD19 molecules

| SEQ ID | | |
|---|---|---|
| NO | Region | Sequence |
| CTL019 | | |
| 760 | HCDR1 | DYGVS |
| | (Kabat) | |
| 761 | HCDR2 | VIWGSETTYYNSALKS |
| | (Kabat) | |
| 762 | HCDR3 | HYYYGGSYAMDY |
| | (Kabat) | |
| 763 | LCDR1 | RASQDISKYLN |
| | (Kabat) | |
| 764 | LCDR2 | HTSRLHS |
| | (Kabat) | |
| 765 | LCDR3 | QQGNTLPYT |
| | (Kabat) | |
| 766 | CTL019 | MALPVTALLLPLALLLHAARPdiqmtqttsslsaslgdrvtiscrasqdis |
| | Full amino | kylnwyqqkpdgtvklliyhtsrlhsgvpsrfsgsgsgtdysltisnleqediatyfcqqg |
| | acid | ntlpytfgggtkleitggggsggggggggsevklqesgpglvapsqslsvtctvsgvsl |
| | sequence | pdygvswirqpprkglewlgviwgsettyynsalksrltiikdnsksqvflkmnslqtd |
| | | dtaiyycakhyyyggsyamdywgqgtsvtvsstttpaprpptpaptiasqplslrpeac |
| | | rpaaggavhtrgldfacdiyiwaplagtcgvlllslvitlyckrgrkkllyifkqpfmrpvq |
| | | ttqeedgccrfpeeeeggcelrvkfsrsadapaykqgqnqlynelnlgrreeydvldkr |
| | | rgrdpemggkprrknpqeglynelqkdkmaeayseigmkgerrrgkghdglyqgls |
| | | tatkdtydalhmqalppr |
| 685 | CTL019 | atggccttaccagtgaccgccttgctcctgccgctggccttgctgctccacgccgccaggc |
| | Full | cggacatccagatgacacagactacatcctccctgtctgcctctctgggagacagagtcac |
| | nucleotide | catcagttgcagggcaagtcaggacattagtaaatatttaaattggtatcagcagaaaccag |
| | sequence | atggaactgttaaactcctgatctaccatacatcaagattacactcaggagtcccatcaaggt |
| | | tcagtggcagtgggtctggaacagattattctctcaccattagcaacctggagcaagaagat |
| | | attgccacttacttttgccaacagggtaatacgcttccgtacacgttcggaggggggaccaa |
| | | gctggagatcacaggtggcggtggctcgggcggtggtgggtcgggtggcggcggatct |
| | | gaggtgaaactgcaggagtcaggacctggcctggtggcgccctcacagagcctgtccgt |
| | | cacatgcactgtctcaggggtctcattacccgactatggtgtaagctggattcgccagcctc |
| | | cacgaaagggtctggagtggctgggagtaatatggggtagtgaaaccacatactataattc |
| | | agctctcaaatccagactgaccatcatcaaggacaactccaagagccaagttttcttaaaaat |
| | | gaacagtctgcaaactgatgacacagccatttactactgtgccaaacattattactacggtgg |
| | | tagctatgctatggactactggggccaaggaacctcagtcaccgtctcctcaaccacgacg |
| | | ccagcgccgcgaccaccaacaccggcgcccaccatcgcgtcgcagcccctgtccctgc |
| | | gcccagaggcgtgccggccagcggggggggcgcagtgcacacgagggggctggac |
| | | ttcgcctgtgatatctacatctgggcgcccttggccgggacttgtggggtccttctcctgtcac |
| | | tggttatcaccctttactgcaaacggggcagaaagaaactcctgtatatattcaaacaaccat |
| | | ttatgagaccagtacaaactactcaagaggaagatggctgtagctgccgatttccagaaga |
| | | agaagaaggaggatgtgaactgagagtgaagttcagcaggagcgcagacgcccccgcg |
| | | tacaagcagggccagaaccagctctataacgagctcaatctaggacgaagagaggagta |
| | | cgatgttttggacaagagacgtggccgggaccctgagatggggggaaagccgagaagg |
| | | aagaaccctcaggaaggcctgtacaatgaactgcagaaagataagatggcggaggccta |
| | | cagtgagattgggatgaaaggcgagcgccggaggggcaaggggcacgatggcctttac |
| | | cagggtctcagtacagccaccaaggacacctacgacgcccttcacatgcaggccctgccc |
| | | cctcgc |
| 686 | CTL019 | Diqmtqttsslsaslgdrvtiscrasqdiskylnwyqqkpdgtvklliyhtsrlhsgvpsrf |
| | scFv | sgsgsgtdysltisnleqediatyfcqqgntlpytfgggtkleitggggsggggsggggse |
| | domain | vklqesgpglvapsqslsvtctvsgvslpdygvswirqpprkglewlgviwgsettyyn |
| | | salksrltiikdnsksqvflkmnslqtddtaiyycakhyyyggsyamdywgqgtsvtv |
| | | ss |
| Humanized | | |
| CAR2 | | |
| 760 | HCDR1 | DYGVS |
| | (Kabat) | |
| 687 | HCDR2 | VIWGSETTYYQSSLKS |
| | (Kabat) | |
| 762 | HCDR3 | HYYYGGSYAMDY |
| | (Kabat) | |
| 763 | LCDR1 | RASQDISKYLN |
| | (Kabat) | |
| 764 | LCDR2 | HTSRLHS |
| | (Kabat) | |
| 76— | LCDR3 | QQGNTLPYT |
| | (Kabat) | |
| 758 | CAR2 scFv | EIVMTQSPATLSLSPGERATLSCRASQDISKYLNWYQQKP |
| | domain - aa | GQAPRLLIYHTSRLHSGIPARFSGSGSGTDYTLTISSLQPED |
| | (Linker is | FAVYFCQQGNTLPYTFGQGTKLEIKGGGGSGGGGSGGGG |
| | underlined) | SQVQLQESGPGLVKPSETLSLTCTVSGVSLPDYGVSWIRQ |
| | | PPGKGLEWIGVIWGSETTYYQSSLKSRVTISKDNSKNQVS |
| | | LKLSSVTAADTAV- |
| | | YCAKHYYYGGSYAMDYWGQGTLVTVSS |
| 688 | CAR2 scFv | atggccctccctgtcaccgccctgctgcttccgctggctcttctgctccacgccgctcggcc |
| | domain - nt | cgaaattgtgatgacccagtcacccgccactcttagcctttcacccggtgagcgcgcaacc |
| | | ctgtcttgcagagcctcccaagacatctcaaaataccttaattggtatcaacagaagcccgg |
| | | acaggctcctcgccttctgatctaccacaccagccggctccattctggaatccctgccaggt |
| | | tcagcggtagcggatctgggaccgactacaccctcactatcagctcactgcagccagagg |
| | | acttcgctgtctatttctgtcagcaagggaacaccctgccctacacctttggacagggcacc |
| | | aagctcgagattaaaggtggaggtggcagcggaggaggtgggtccggcggtggaggaa |
| | | gccaggtccaactccaagaaagcggaccgggtcttgtgaagccatcagaaactctttcact |
| | | gacttgtactgtgagcggagtgtctctccccgattacggggtgtcttggatcagacagccac |
| | | cggggaagggtctggaatggattggagtgatttggggctctgagactacttactaccaatca |
| | | tccctcaagtcacgcgtcaccatctcaaaggacaactctaagaatcaggtgtcactgaaact |
| | | gtcatctgtgaccgcagccgacaccgccgtgtactattgcgctaagcattactattatggcg |
| | | ggagctacgcaatggattactggggacagggtactctggtcaccgt- |
| | | tccagccaccaccatcatcaccatcaccat |
| 689 | CAR 2 - | MALPVTALLLPLALLLHAARPEIVMTQSPATLSLSPGERA |
| | Full - aa | TLSCRASQDISKYLNWYQQKPGQAPRLLIYHTSRLHSGIP |
| | | ARFSGSGSGTDYTLTISSLQPEDFAVYFCQQGNTLPYTFG |
| | | QGTKLEIKGGGGSGGGGSGGGGSQVQLQESGPGLVKPSE |
| | | TLSLTCTVSGVSLPDYGVSWIRQPPGKGLEWIGVIWGSET |
| | | TYYQSSLKSRVTISKDNSKNQVSLKLSSVTAADTAVYYC |
| | | AKHYYYGGSYAMDYWGQGTLVTVSSTTTPAPRPPTPAP |
| | | TIASQPLSLRPEACRPAAGGAVHTRGLDFACDIYIWAPLA |
| | | GTCGVLLLSLVITLYCKRGRKKLLYIFKQPFMRPVQTTQE |
| | | EDGCSCRFPEEEEGGCELRVKFSRSADAPAYKQGQNQLY |
| | | NELNLGRREEYDVLDKRRGRDPEMGGKPRRKNPQEGLY |
| | | NELQKDKMAEAYSEIGMKGERR- |
| | | GKGHDGLYQGLSTATKDTYDALHMQALPPR |
| 690 | CAR 2 - | atggccctccctgtcaccgccctgctgcttccgctggctcttctgctccacgccgctcggcc |
| | Full - nt | cgaaattgtgatgacccagtcacccgccactcttagcctttcacccggtgagcgcgcaacc |
| | | ctgtcttgcagagcctcccaagacatctcaaaataccttaattggtatcaacagaagcccgg |
| | | acaggctcctcgccttctgatctaccacaccagccggctccattctggaatccctgccaggt |
| | | tcagcggtagcggatctgggaccgactacaccctcactatcagctcactgcagccagagg |
| | | acttcgctgtctatttctgtcagcaagggaacaccctgccctacacctttggacagggcacc |
| | | aagctcgagattaaaggtggaggtggcagcggaggaggtgggtccggcggtggaggaa |
| | | gccaggtccaactccaagaaagcggaccgggtcttgtgaagccatcagaaactctttcact |
| | | gacttgtactgtgagcggagtgtctctccccgattacggggtgtcttggatcagacagccac |
| | | cggggaagggtctggaatggattggagtgatttggggctctgagactacttactaccaatca |
| | | tccctcaagtcacgcgtcaccatctcaaaggacaactctaagaatcaggtgtcactgaaact |
| | | gtcatctgtgaccgcagccgacaccgccgtgtactattgcgctaagcattactattatggcg |
| | | ggagctacgcaatggattactggggacagggtactctggtcaccgtgtccagcaccactac |
| | | cccagcaccgaggccacccaccccggctcctaccatcgcctcccagcctctgtccctgcg |
| | | tccggaggcatgtagacccgcagctggtggggccgtgcatacccggggtcttgacttcgc |
| | | ctgcgatatctacatttgggcccctctggctggtacttgcggggtcctgctgctttcactcgtg |
| | | atcactctttactgtaagcgcggtcggaagaagctgctgtacatctttaagcaacccttcatg |
| | | aggcctgtgcagactactcaagaggaggacggctgttcatgccggttcccagaggagga |
| | | ggaaggcggctgcgaactgcgcgtgaaattcagccgcagcgcagatgctccagcctaca |
| | | agcaggggcagaaccagctctacaacgaactcaatcttggtcggagagaggagtacgac |
| | | gtgctggacaagcggagaggacgggacccagaaatgggcgggaagccgcgcagaaa |
| | | gaatccccaagagggcctgtacaacgagctccaaaaggataagatggcagaagcctata |
| | | gcgagattggtatgaaaggggaacgcagaagaggcaaaggccacgacggactgtacca |
| | | gggactcagcaccgccaccaaggacacctatgacgctcttcacatgcaggccctgccgcc |
| | | tcgg |
| 349 | CAR 2A- | MALPVTALLLPLALLLHAARPEIVMTQSPATLSLSPGERATLSCRASQ |
| | Full amino | DISKYLNWYQQKPGQAPRLLIYHTSRLHSGIPARFSGSGSGTDYTLTIS |
| | acid | SLQPEDFAVYFCQQGNTLPYTFGQGTKLEIKGGGGSGGGGSGGGGSQ |
| | sequence; | VQLQESGPGLVKPSETLSLTCTVSGVSLPDYGVSWIRQPPGKGLEWIG |
| | signal | VIWGSETTYYQSSLKSRVTISKDNSKNQVSLKLSSVTAADTAVYYCA |
| | peptide | KHYYYGGSYAMDYWGQGTLVTVSSTTTPAPRPPTPAPTIASQPLSLRP |
| | underlined | EACRPAAGGAVHTRGLDFACDIYIWAPLAGTCGVLLLSLVITLYCKR |
| | | GRKKLLYIFKQPFMRPVQTTQEEDGCSCRFPEEEEGGCELRVKFSRSA |
| | | DAPAYQQGQNQLYNELNLGRREEYDVLDKRRGRDPEMGGKPRRKN |
| | | PQEGLYNELQKDKMAEAYSEIGMKGERRRGKGHDGLYQGLSTATKD |
| | | TYDALHMQALPPR |
| 225 | CAR 2A - | EIVMTQSPATLSLSPGERATLSCRASQDISKYLNWYQQKPGQAPRLLI |
| | amino acid | YHTSRLHSGIPARFSGSGSGTDYTLTISSLQPEDFAVYFCQQGNTLPYT |
| | sequence; | FGQGTKLEIKGGGGSGGGGSGGGGSQVQLQESGPGLVKPSETLSLTC |
| | no signal | TVSGVSLPDYGVSWIRQPPGKGLEWIGVIWGSETTYYQSSLKSRVTIS |
| | peptide | KDNSKNQVSLKLSSVTAADTAVYYCAKHYYYGGSYAMDYWGQGT |
| | | LVTVSSTTTPAPRPPTPAPTIASQPLSLRPEACRPAAGGAVHTRGLDFA |
| | | CDIYIWAPLAGTCGVLLLSLVITLYCKRGRKKLLYIFKQPFMRPVQTT |
| | | QEEDGCSCRFPEEEEGGCELRVKFSRSADAPAYQQGQNQLYNELNLG |
| | | RREEYDVLDKRRGRDPEMGGKPRRKNPQEGLYNELQKDKMAEAYS |
| | | EIGMKGERRRGKGHDGLYQGLSTATKDTYDALHMQALPPR |
| 354 | CAR 2A | atggccctccctgtcaccgccctgctgcttccgctggctcttctgctccacgccgctcggcccgaaattgtgatg |
| | full nucleic | acccagtcacccgccactcttagcctttcacccggtgagcgcgcaaccctgtcttgcagagcctcccaagacat |
| | acid | ctcaaaataccttaattggtatcaacagaagcccggacaggctcctcgccttctgatctaccacaccagccggc |
| | sequence; | tccattctggaatccctgccaggttcagcggtagcggatctgggaccgactacaccctcactatcagctcactg |
| | signal | cagccagaggacttcgctgtctatttctgtcagcaagggaacaccctgccctacacctttggacagggcaccaa |
| | peptide and | gctcgagattaaaggtggaggtggcagcggaggaggtgggtccggcggtggaggaagccaggtccaactc |
| | stop codon | caagaaagcggaccgggtcttgtgaagccatcagaaactctttcactgacttgtactgtgagcggagtgtctctc |
| | underlined | cccgattacggggtgtcttggatcagacagccaccggggaagggtctggaatggattggagtgatttggggct |
| | | ctgagactacttactaccaatcatccctcaagtcacgcgtcaccatctcaaaggacaactctaagaatcaggtgt |
| | | cactgaaactgtcatctgtgaccgcagccgacaccgccgtgtactattgcgctaagcattactattatggcggga |
| | | gctacgcaatggattactggggacagggtactctggtcaccgtgtccagcaccactaccccagcaccgaggc |
| | | cacccaccccggctcctaccatcgcctcccagcctctgtccctgcgtccggaggcatgtagacccgcagctg |
| | | gtggggccgtgcatacccggggtcttgacttcgcctgcgatatctacatttgggcccctctggctggtacttgcg |
| | | gggtcctgctgctttcactcgtgatcactctttactgtaagcgcggtcggaagaagctgctgtacatctttaagca |
| | | acccttcatgaggcctgtgcagactactcaagaggaggacggctgttcatgccggttcccagaggaggagga |
| | | aggcggctgcgaactgcgcgtgaaattcagccgcagcgcagatgctccagcctaccagcaggggcagaac |
| | | cagctctacaacgaactcaatcttggtcggagagaggagtacgacgtgctggacaagcggagaggacggga |
| | | cccagaaatgggcgggaagccgcgcagaaagaatccccaagagggcctgtacaacgagctccaaaaggat |
| | | aagatggcagaagcctatagcgagattggtatgaaaggggaacgcagaagaggcaaaggccacgacggac |
| | | tgtaccagggactcagcaccgccaccaaggacacctatgacgctcttcacatgcaggccctgccgcctcggt |
| | | aa |
| 355 | CAR 2A | atggccctccctgtcaccgccctgctgcttccgctggctcttctgctccacgccgctcggcccgaaattgtgatg |
| | nucleic | acccagtcacccgccactcttagcctttcacccggtgagcgcgcaaccctgtcttgcagagcctcccaagacat |
| | acid | ctcaaaataccttaattggtatcaacagaagcccggacaggctcctcgccttctgatctaccacaccagccggc |
| | sequence; | tccattctggaatccctgccaggttcagcggtagcggatctgggaccgactacaccctcactatcagctcactg |
| | signal | cagccagaggacttcgctgtctatttctgtcagcaagggaacaccctgccctacacctttggacagggcaccaa |
| | peptide | gctcgagattaaaggtggaggtggcagcggaggaggtgggtccggcggtggaggaagccaggtccaactc |
| | underlined; | caagaaagcggaccgggtcttgtgaagccatcagaaactctttcactgacttgtactgtgagcggagtgtctctc |
| | no stop | cccgattacggggtgtcttggatcagacagccaccggggaagggtctggaatggattggagtgatttggggct |
| | codon | ctgagactacttactaccaatcatccctcaagtcacgcgtcaccatctcaaaggacaactctaagaatcaggtgt |
| | | cactgaaactgtcatctgtgaccgcagccgacaccgccgtgtactattgcgctaagcattactattatggcggga |
| | | gctacgcaatggattactggggacagggtactctggtcaccgtgtccagcaccactaccccagcaccgaggc |
| | | cacccaccccggctcctaccatcgcctcccagcctctgtccctgcgtccggaggcatgtagacccgcagctg |
| | | gtggggccgtgcatacccggggtcttgacttcgcctgcgatatctacatttgggcccctctggctggtacttgcg |
| | | gggtcctgctgctttcactcgtgatcactctttactgtaagcgcggtcggaagaagctgctgtacatctttaagca |
| | | acccttcatgaggcctgtgcagactactcaagaggaggacggctgttcatgccggttcccagaggaggagga |
| | | aggcggctgcgaactgcgcgtgaaattcagccgcagcgcagatgctccagcctaccagcaggggcagaac |
| | | cagctctacaacgaactcaatcttggtcggagagaggagtacgacgtgctggacaagcggagaggacggga |
| | | cccagaaatggggggaagccgcgcagaaagaatccccaagagggcctgtacaacgagctccaaaaggat |
| | | aagatggcagaagcctatagcgagattggtatgaaaggggaacgcagaagaggcaaaggccacgacggac |
| | | tgtaccagggactcagcaccgccaccaaggacacctatgacgctcttcacatgcaggccctgccgcctcgg |
| 356 | CAR 2A | gaaattgtgatgacccagtcacccgccactcttagcctttcacccggtgagcgcgcaaccctgtcttgcagagc |
| | nucleic | ctcccaagacatctcaaaataccttaattggtatcaacagaagcccggacaggctcctcgccttctgatctacca |
| | acid | caccagccggctccattctggaatccctgccaggttcagcggtagcggatctgggaccgactacaccctcact |
| | sequence; | atcagctcactgcagccagaggacttcgctgtctatttctgtcagcaagggaacaccctgccctacacctttgga |
| | no signal | cagggcaccaagctcgagattaaaggtggaggtggcagcggaggaggtgggtccggcggtggaggaagc |
| | peptide; | caggtccaactccaagaaagcggaccgggtcttgtgaagccatcagaaactctttcactgacttgtactgtgag |
| | stop codon | cggagtgtctctccccgattacggggtgtcttggatcagacagccaccggggaagggtctggaatggattgga |
| | underlined | gtgatttggggctctgagactacttactaccaatcatccctcaagtcacgcgtcaccatctcaaaggacaactcta |
| | | agaatcaggtgtcactgaaactgtcatctgtgaccgcagccgacaccgccgtgtactattgcgctaagcattact |
| | | attatggcgggagctacgcaatggattactggggacagggtactctggtcaccgtgtccagcaccactacccc |
| | | agcaccgaggccacccaccccggctcctaccatcgcctcccagcctctgtccctgcgtccggaggcatgtag |
| | | acccgcagctggtggggccgtgcatacccggggtcttgacttcgcctgcgatatctacatttgggcccctctgg |
| | | ctggtacttgcggggtcctgctgctttcactcgtgatcactctttactgtaagcgcggtcggaagaagctgctgta |
| | | catctttaagcaacccttcatgaggcctgtgcagactactcaagaggaggacggctgttcatgccggttcccag |
| | | aggaggaggaaggcggctgcgaactgcgcgtgaaattcagccgcagcgcagatgctccagcctaccagca |
| | | ggggcagaaccagctctacaacgaactcaatcttggtcggagagaggagtacgacgtgctggacaagcgga |
| | | gaggacgggacccagaaatgggcgggaagccgcgcagaaagaatccccaagagggcctgtacaacgag |
| | | ctccaaaaggataagatggcagaagcctatagcgagattggtatgaaaggggaacgcagaagaggcaaag |
| | | gccacgacggactgtaccagggactcagcaccgccaccaaggacacctatgacgctcttcacatgcaggcc |
| | | ctgccgcctcggtaa |
| 417 | CAR 2A | gaaattgtgatgacccagtcacccgccactcttagcctttcacccggtgagcgcgcaaccct |
| | nucleic | gtcttgcagagcctcccaagacatctcaaaataccttaattggtatcaacagaagcccggac |
| | acid | aggctcctcgccttctgatctaccacaccagccggctccattctggaatccctgccaggttc |
| | sequence; | agcggtagcggatctgggaccgactacaccctcactatcagctcactgcagccagaggac |
| | no signal | ttcgctgtctatttctgtcagcaagggaacaccctgccctacacctttggacagggcaccaa |
| | peptide; no | gctcgagattaaaggtggaggtggcagcggaggaggtgggtccggcggtggaggaagc |
| | stop codon | caggtccaactccaagaaagcggaccgggtcttgtgaagccatcagaaactctttcactga |
| | | cttgtactgtgagcggagtgtctctccccgattacggggtgtcttggatcagacagccaccg |
| | | gggaagggtctggaatggattggagtgatttggggctctgagactacttactaccaatcatc |
| | | cctcaagtcacgcgtcaccatctcaaaggacaactctaagaatcaggtgtcactgaaactgt |
| | | catctgtgaccgcagccgacaccgccgtgtactattgcgctaagcattactattatggcggg |
| | | agctacgcaatggattactggggacagggtactctggtcaccgtgtccagcaccactaccc |
| | | cagcaccgaggccacccaccccggctcctaccatcgcctcccagcctctgtccctgcgtc |
| | | cggaggcatgtagacccgcagctggtggggccgtgcatacccggggtcttgacttcgcct |
| | | gcgatatctacatttgggcccctctggctggtacttgcggggtcctgctgctttcactcgtgat |
| | | cactctttactgtaagcgcggtcggaagaagctgctgtacatctttaagcaacccttcatgag |
| | | gcctgtgcagactactcaagaggaggacggctgttcatgccggttcccagaggaggagg |
| | | aaggcggctgcgaactgcgcgtgaaattcagccgcagcgcagatgctccagcctaccag |
| | | caggggcagaaccagctctacaacgaactcaatcttggtcggagagaggagtacgacgt |
| | | gctggacaagcggagaggacgggacccagaaatgggcgggaagccgcgcagaaaga |
| | | atccccaagagggcctgtacaacgagctccaaaaggataagatggcagaagcctatagc |
| | | gagattggtatgaaaggggaacgcagaagaggcaaaggccacgacggactgtaccagg |
| | | gactcagcaccgccaccaaggacacctatgacgctcttcacatgcaggccctgccgcctc |
| | | gg |
| 250 | Anti-CD19 | QVQLQESGPGLVKPSETLSLTCTVSGVSLPDYGVSWIRQPPGKGLEWI |
| | VH | GVIWGSETTYYQSSLKSRVTISKDNSKNQVSLKLSSVTAADTAVYYC |
| | | AKHYYYGGSYAMDYWGQGTLVTVSS |
| 251 | Anti-CD19 | EIVMTQSPATLSLSPGERATLSCRASQDISKYLNWYQQKPGQAPRLLI |
| | VL | YHTSRLHSGIPARFSGSGSGTDYTLTISSLQPEDFAVYFCQQGNTLPYT |
| | | FGQGTKLEIK |
| 331 | VH | QVQLQESGPGLVKPSETLSLTCTVSGVSLPDYGVSWIRQPPGKCLEWI |
| | | GVIWGSETTYYQSSLKSRVTISKDNSKNQVSLKLSSVTAADTAVYYC |
| | | AKHYYYGGSYAMDYWGQGTLVTVSS |
| 332 | VL | EIVMTQSPATLSLSPGERATLSCRASQDISKYLNWYQQKPGQAPRLLI |
| | | YHTSRLHSGIPARFSGSGSGTDYTLTISSLQPEDFAVYFCQQGNTLPYT |
| | | FGCGTKLEIK |

BCMA

A non-limiting exemplary tumor antigen is BCMA. CARs that bind to BCMA are known in the art. For example, those disclosed WO2016/014565 or WO2019/241426. Any known BCMA CAR, for example, the BCMA antigen binding domain of any known BCMA CAR, in the art can be used in accordance with the present disclosure. For example, BCMA-1, BCMA-2, BCMA-3, BCMA-4, BCMA-5, BCMA-6, BCMA-7, BCMA-8, BCMA-9, BCMA-10, BCMA-11, BCMA-12, BCMA-13, BCMA-14, BCMA-15, 149362, 149363, 149364, 149365, 149366, 149367, 149368, 149369, BCMA_EBB-C1978-A4, BCMA_EBB-C1978-G1, BCMA_EBB-C1979-C1, BCMA_EBB-C1978-C7, BCMA_EBB-C1978-D10, BCMA_EBB-C1979-C12, BCMA_EBB-C1980-G4, BCMA_EBB-C1980-D2, BCMA_EBB-C1978-A10, BCMA_EBB-C1978-D4, BCMA_EBB-C1980-A2, BCMA_EBB-C1981-C3, BCMA_EBB-C1978-G4, A71D12.2, C11D5.3, C12A3.2, or C13F12.1, disclosed in WO2016/014565.

In some embodiments, the BCMA CAR comprises one or more CDRs, VH, VL, scFv, or full-length sequences of BCMA-1, BCMA-2, BCMA-3, BCMA-4, BCMA-5, BCMA-6, BCMA-7, BCMA-8, BCMA-9, BCMA-10, BCMA-11, BCMA-12, BCMA-13, BCMA-14, BCMA-15, 149362, 149363, 149364, 149365, 149366, 149367, 149368, 149369, BCMA_EBB-C1978-A4, BCMA_EBB-C1978-G1, BCMA_EBB-C1979-C1, BCMA_EBB-C1978-C7, BCMA_EBB-C1978-D10, BCMA_EBB-C1979-C12, BCMA_EBB-C1980-G4, BCMA_EBB-C1980-D2, BCMA_EBB-C1978-A10, BCMA_EBB-C1978-D4, BCMA_EBB-C1980-A2, BCMA_EBB-C1981-C3, BCMA_EBB-C1978-G4, A71D12.2, C11D5.3, C12A3.2, or C13F12.1 disclosed in WO2016/014565, or a sequence substantially (for example, 95-99%) identical thereto.

In some embodiments, a BCMA CAR comprises a sequence, for example, a CDR, VH, VL, scFv, or full-CAR sequence, disclosed in Tables 2-14, or a sequence having at least 80%, 85%, 90%, 95%, or 99% identity thereto.

TABLE 2
Amino acid and nucleic acid sequences of exemplary PALLAS-derived anti-
BCMA molecules

| SEQ | Name/ | |
|---|---|---|
| ID NO | Description | Sequence |
| R1B6 | | |
| SEQ ID | HCDR1 | SYAMS |
| NO: 44 | (Kabat) | |
| SEQ ID | HCDR2 | AISGSGGSTYYADSVKG |
| NO: 45 | (Kabat) | |
| SEQ ID | HCDR3 | REWVPYDVSWYFDY |
| NO: 46 | (Kabat) | |
| SEQ ID | HCDR1 | GFTFSSY |
| NO: 47 | (Chothia) | |
| SEQ ID | HCDR2 | SGSGGS |
| NO: 48 | (Chothia) | |
| SEQ ID | HCDR3 | REWVPYDVSWYFDY |
| NO: 46 | (Chothia) | |
| SEQ ID | HCDR1 | GFTFSSYA |
| NO: 49 | (IMGT) | |
| SEQ ID | HCDR2 | ISGSGGST |
| NO: 50 | (IMGT) | |
| SEQ ID | HCDR3 | ARREWVPYDVSWYFDY |
| NO: 51 | (IMGT) | |
| SEQ ID | VH | EVQLLESGGGLVQPGGSLRLSCAASGFTFSSYAMSWVRQAP |
| NO: 52 | | GKGLEWVSAISGSGGSTYYADSVKGRFTISRDNSKNTLYLQ |
| | | MNSLRAEDTAVYYCARREWVPYDVSWYFDYWGQGTLVT |
| | | VSS |
| SEQ ID | DNA VH | GAAGTGCAGTTGCTGGAGTCAGGCGGAGGACTGGTGCAG |
| NO: 53 | | CCCGGAGGATCGCTTCGCTTGAGCTGCGCAGCCTCAGGCT |
| | | TTACCTTCTCCTCCTACGCCATGTCCTGGGTCAGACAGGC |
| | | TCCCGGGAAGGGACTGGAATGGGTGTCCGCCATTAGCGG |
| | | TTCCGGCGGAAGCACTTACTATGCCGACTCTGTGAAGGG |
| | | CCGCTTCACTATCTCCCGGGACAACTCCAAGAACACCCTG |
| | | TATCTCCAAATGAATTCCCTGAGGGCCGAAGATACCGCG |
| | | GTGTACTACTGCGCTAGACGGGAGTGGGTGCCCTACGAT |
| | | GTCAGCTGGTACTTCGACTACTGGGGACAGGGCACTCTC |
| | | GTGACTGTGTCCTCC |
| SEQ ID | LCDR1 | RASQSISSYLN |
| NO: 54 | (Kabat) | |
| SEQ ID | LCDR2 | AASSLQS |
| NO: 55 | (Kabat) | |
| SEQ ID | LCDR3 | QQSYSTPLT |
| NO: 56 | (Kabat) | |
| SEQ ID | LCDR1 | SQSISSY |
| NO: 57 | (Chothia) | |
| SEQ ID | LCDR2 | AAS |
| NO: 58 | (Chothia) | |
| SEQ ID | LCDR3 | SYSTPL |
| NO: 59 | (Chothia) | |
| SEQ ID | LCDR1 | QSISSY |
| NO: 60 | (IMGT) | |
| SEQ ID | LCDR2 | AAS |
| NO: 58 | (IMGT) | |
| SEQ ID | LCDR3 | QQSYSTPLT |
| NO: 56 | (IMGT) | |
| SEQ ID | VL | DIQMTQSPSSLSASVGDRVTITCRASQSISSYLNWYQQKPGK |
| NO: 61 | | APKLLIYAASSLQSGVPSRFSGSGSGTDFTLTISSLQPEDFAT |
| | | YYCQQSYSTPLTFGQGTKVEIK |
| SEQ ID | DNA VL | GACATTCAAATGACTCAGTCCCCGTCCTCCCTCTCCGCCT |
| NO: 62 | | CCGTGGGAGATCGCGTCACGATCACGTGCAGGGCCAGCC |
| | | AGAGCATCTCCAGCTACCTGAACTGGTACCAGCAGAAGC |
| | | CAGGGAAGGCACCGAAGCTCCTGATCTACGCCGCTAGCT |
| | | CGCTGCAGTCCGGCGTCCCTTCACGGTTCTCGGGATCGGG |
| | | CTCAGGCACCGACTTCACCCTGACCATTAGCAGCCTGCAG |
| | | CCGGAGGACTTCGCGACATACTACTGTCAGCAGTCATACT |
| | | CCACCCCTCTGACCTTCGGCCAAGGGACCAAAGTGGAGA |
| | | TCAAG |
| SEQ ID | Linker | GGGGSGGGGSGGGGSGGGGS |
| NO: 63 | | |
| SEQ ID | scFv (VH- | EVQLLESGGGLVQPGGSLRLSCAASGFTFSSYAMSWVRQAP |
| NO: 64 | linker-VL) | GKGLEWVSAISGSGGSTYYADSVKGRFTISRDNSKNTLYLQ |
| | | MNSLRAEDTAVYYCARREWVPYDVSWYFDYWGQGTLVT |
| | | VSSGGGGSGGGGSGGGGSGGGGSDIQMTQSPSSLSASVGDR |
| | | VTITCRASQSISSYLNWYQQKPGKAPKLLIYAASSLQSGVPS |
| | | RFSGSGSGTDFTLTISSLQPEDFATYYCQQSYSTPLTFGQGTK |
| | | VEIK |
| SEQ ID | DNA scFv | GAAGTGCAGTTGCTGGAGTCAGGCGGAGGACTGGTGCAG |
| NO: 65 | | CCCGGAGGATCGCTTCGCTTGAGCTGCGCAGCCTCAGGCT |
| | | TTACCTTCTCCTCCTACGCCATGTCCTGGGTCAGACAGGC |
| | | TCCCGGGAAGGGACTGGAATGGGTGTCCGCCATTAGCGG |
| | | TTCCGGCGGAAGCACTTACTATGCCGACTCTGTGAAGGG |
| | | CCGCTTCACTATCTCCCGGGACAACTCCAAGAACACCCTG |
| | | TATCTCCAAATGAATTCCCTGAGGGCCGAAGATACCGCG |
| | | GTGTACTACTGCGCTAGACGGGAGTGGGTGCCCTACGAT |
| | | GTCAGCTGGTACTTCGACTACTGGGGACAGGGCACTCTC |
| | | GTGACTGTGTCCTCCGGTGGTGGTGGATCGGGGGGTGGT |
| | | GGTTCGGGCGGAGGAGGATCTGGAGGAGGAGGGTCGGA |
| | | CATTCAAATGACTCAGTCCCCGTCCTCCCTCTCCGCCTCC |
| | | GTGGGAGATCGCGTCACGATCACGTGCAGGGCCAGCCAG |
| | | AGCATCTCCAGCTACCTGAACTGGTACCAGCAGAAGCCA |
| | | GGGAAGGCACCGAAGCTCCTGATCTACGCCGCTAGCTCG |
| | | CTGCAGTCCGGCGTCCCTTCACGGTTCTCGGGATCGGGCT |
| | | CAGGCACCGACTTCACCCTGACCATTAGCAGCCTGCAGC |
| | | CGGAGGACTTCGCGACATACTACTGTCAGCAGTCATACTC |
| | | CACCCCTCTGACCTTCGGCCAAGGGACCAAAGTGGAGAT |
| | | CAAG |
| SEQ ID | Full CAR | EVQLLESGGGLVQPGGSLRLSCAASGFTFSSYAMSWVRQAP |
| NO: 66 | amino acid | GKGLEWVSAISGSGGSTYYADSVKGRFTISRDNSKNTLYLQ |
| | sequence | MNSLRAEDTAVYYCARREWVPYDVSWYFDYWGQGTLVT |
| | | VSSGGGGSGGGGSGGGGSGGGGSDIQMTQSPSSLSASVGDR |
| | | VTITCRASQSISSYLNWYQQKPGKAPKLLIYAASSLQSGVPS |
| | | RFSGSGSGTDFTLTISSLQPEDFATYYCQQSYSTPLTFGQGTK |
| | | VEIKTTTPAPRPPTPAPTIASQPLSLRPEACRPAAGGAVHTRG |
| | | LDFACDIYIWAPLAGTCGVLLLSLVITLYCKRGRKKLLYIFK |
| | | QPFMRPVQTTQEEDGCSCRFPEEEEGGCELRVKFSRSADAP |
| | | AYQQGQNQLYNELNLGRREEYDVLDKRRGRDPEMGGKPR |
| | | RKNPQEGLYNELQKDKMAEAYSEIGMKGERRRGKGHDGL |
| | | YQGLSTATKDTYDALHMQALPPR |
| SEQ ID | Full CAR | GAAGTGCAGTTGCTGGAGTCAGGCGGAGGACTGGTGCAG |
| NO: 67 | DNA | CCCGGAGGATCGCTTCGCTTGAGCTGCGCAGCCTCAGGCT |
| | sequence | TTACCTTCTCCTCCTACGCCATGTCCTGGGTCAGACAGGC |
| | | TCCCGGGAAGGGACTGGAATGGGTGTCCGCCATTAGCGG |
| | | TTCCGGCGGAAGCACTTACTATGCCGACTCTGTGAAGGG |
| | | CCGCTTCACTATCTCCCGGGACAACTCCAAGAACACCCTG |
| | | TATCTCCAAATGAATTCCCTGAGGGCCGAAGATACCGCG |
| | | GTGTACTACTGCGCTAGACGGGAGTGGGTGCCCTACGAT |
| | | GTCAGCTGGTACTTCGACTACTGGGGACAGGGCACTCTC |
| | | GTGACTGTGTCCTCCGGTGGTGGTGGATCGGGGGGTGGT |
| | | GGTTCGGGCGGAGGAGGATCTGGAGGAGGAGGGTCGGA |
| | | CATTCAAATGACTCAGTCCCCGTCCTCCCTCTCCGCCTCC |
| | | GTGGGAGATCGCGTCACGATCACGTGCAGGGCCAGCCAG |
| | | AGCATCTCCAGCTACCTGAACTGGTACCAGCAGAAGCCA |
| | | GGGAAGGCACCGAAGCTCCTGATCTACGCCGCTAGCTCG |
| | | CTGCAGTCCGGCGTCCCTTCACGGTTCTCGGGATCGGGCT |
| | | CAGGCACCGACTTCACCCTGACCATTAGCAGCCTGCAGC |
| | | CGGAGGACTTCGCGACATACTACTGTCAGCAGTCATACTC |
| | | CACCCCTCTGACCTTCGGCCAAGGGACCAAAGTGGAGAT |
| | | CAAGACCACTACCCCAGCACCGAGGCCACCCACCCCGGC |
| | | TCCTACCATCGCCTCCCAGCCTCTGTCCCTGCGTCCGGAG |
| | | GCATGTAGACCCGCAGCTGGTGGGGCCGTGCATACCCGG |
| | | GGTCTTGACTTCGCCTGCGATATCTACATTTGGGCCCCTC |
| | | TGGCTGGTACTTGCGGGGTCCTGCTGCTTTCACTCGTGAT |
| | | CACTCTTTACTGTAAGCGCGGTCGGAAGAAGCTGCTGTAC |
| | | ATCTTTAAGCAACCCTTCATGAGGCCTGTGCAGACTACTC |
| | | AAGAGGAGGACGGCTGTTCATGCCGGTTCCCAGAGGAGG |
| | | AGGAAGGCGGCTGCGAACTGCGCGTGAAATTCAGCCGCA |
| | | GCGCAGATGCTCCAGCCTACCAGCAGGGGCAGAACCAGC |
| | | TCTACAACGAACTCAATCTTGGTCGGAGAGAGGAGTACG |
| | | ACGTGCTGGACAAGCGGAGAGGACGGGACCCAGAAATG |
| | | GGCGGGAAGCCGCGCAGAAAGAATCCCCAAGAGGGCCT |
| | | GTACAACGAGCTCCAAAAGGATAAGATGGCAGAAGCCTA |
| | | TAGCGAGATTGGTATGAAAGGGGAACGCAGAAGAGGCA |
| | | AAGGCCACGACGGACTGTACCAGGGACTCAGCACCGCCA |
| | | CCAAGGACACCTATGACGCTCTTCACATGCAGGCCCTGCC |
| | | GCCTCGG |
| R1F2 | | |
| SEQ ID | HCDR1 | SYAMS |
| NO: 44 | (Kabat) | |
| SEQ ID | HCDR2 | AISGSGGSTYYADSVKG |
| NO: 45 | (Kabat) | |
| SEQ ID | HCDR3 | REWWYDDWYLDY |
| NO: 68 | (Kabat) | |
| SEQ ID | HCDR1 | GFTFSSY |
| NO: 47 | (Chothia) | |
| SEQ ID | HCDR2 | SGSGGS |
| NO: 48 | (Chothia) | |
| SEQ ID | HCDR3 | REWWYDDWYLDY |
| NO: 68 | (Chothia) | |
| SEQ ID | HCDR1 | GFTFSSYA |
| NO: 49 | (IMGT) | |
| SEQ ID | HCDR2 | ISGSGGST |
| NO: 50 | (IMGT) | |
| SEQ ID | HCDR3 | ARREWWYDDWYLDY |
| NO: 69 | (IMGT) | |
| SEQ ID | VH | EVQLLESGGGLVQPGGSLRLSCAASGFTFSSYAMSWVRQAP |
| NO: 70 | | GKGLEWVSAISGSGGSTYYADSVKGRFTISRDNSKNTLYLQ |
| | | MNSLRAEDTAVYYCARREWWYDDWYLDYWGQGTLVTVS |
| | | S |
| SEQ ID | DNA VH | GAAGTGCAGTTGCTGGAGTCAGGCGGAGGACTGGTGCAG |
| NO: 71 | | CCCGGAGGATCGCTTCGCTTGAGCTGCGCAGCCTCAGGCT |
| | | TTACCTTCTCCTCCTACGCCATGTCCTGGGTCAGACAGGC |
| | | TCCCGGGAAGGGACTGGAATGGGTGTCCGCCATTAGCGG |
| | | TTCCGGCGGAAGCACTTACTATGCCGACTCTGTGAAGGG |
| | | CCGCTTCACTATCTCCCGGGACAACTCCAAGAACACCCTG |
| | | TATCTCCAAATGAATTCCCTGAGGGCCGAAGATACCGCG |
| | | GTGTACTACTGCGCTAGACGGGAGTGGTGGTACGACGAT |
| | | TGGTACCTGGACTACTGGGGACAGGGCACTCTCGTGACT |
| | | GTGTCCTCC |
| SEQ ID | LCDR1 | RASQSISSYLN |
| NO: 54 | (Kabat) | |
| SEQ ID | LCDR2 | AASSLQS |
| NO: 55 | (Kabat) | |
| SEQ ID | LCDR3 | QQSYSTPLT |
| NO: 56 | (Kabat) | |
| SEQ ID | LCDR1 | SQSISSY |
| NO: 57 | (Chothia) | |
| SEQ ID | LCDR2 | AAS |
| NO: 58 | (Chothia) | |
| SEQ ID | LCDR3 | SYSTPL |
| NO: 59 | (Chothia) | |
| SEQ ID | LCDR1 | QSISSY |
| NO: 60 | (IMGT) | |
| SEQ ID | LCDR2 | AAS |
| NO: 58 | (IMGT) | |
| SEQ ID | LCDR3 | QQSYSTPLT |
| NO: 56 | (IMGT) | |
| SEQ ID | VL | DIQMTQSPSSLSASVGDRVTITCRASQSISSYLNWYQQKPGK |
| NO: 61 | | APKLLIYAASSLQSGVPSRFSGSGSGTDFTLTISSLQPEDFAT |
| | | YYCQQSYSTPLTFGQGTKVEIK |
| SEQ ID | DNA VL | GACATTCAAATGACTCAGTCCCCGTCCTCCCTCTCCGCCT |
| NO: 62 | | CCGTGGGAGATCGCGTCACGATCACGTGCAGGGCCAGCC |
| | | AGAGCATCTCCAGCTACCTGAACTGGTACCAGCAGAAGC |
| | | CAGGGAAGGCACCGAAGCTCCTGATCTACGCCGCTAGCT |
| | | CGCTGCAGTCCGGCGTCCCTTCACGGTTCTCGGGATCGGG |
| | | CTCAGGCACCGACTTCACCCTGACCATTAGCAGCCTGCAG |
| | | CCGGAGGACTTCGCGACATACTACTGTCAGCAGTCATACT |
| | | CCACCCCTCTGACCTTCGGCCAAGGGACCAAAGTGGAGA |
| | | TCAAG |
| SEQ ID | Linker | GGGGSGGGGSGGGGSGGGGS |
| NO: 63 | | |
| SEQ ID | scFv (VH- | EVQLLESGGGLVQPGGSLRLSCAASGFTFSSYAMSWVRQAP |
| NO: 72 | linker-VL) | GKGLEWVSAISGSGGSTYYADSVKGRFTISRDNSKNTLYLQ |
| | | MNSLRAEDTAVYYCARREWWYDDWYLDYWGQGTLVTVS |
| | | SGGGGSGGGGSGGGGSGGGGSDIQMTQSPSSLSASVGDRVT |
| | | ITCRASQSISSYLNWYQQKPGKAPKLLIYAASSLQSGVPSRF |
| | | SGSGSGTDFTLTISSLQPEDFATYYCQQSYSTPLTFGQGTKV |
| | | EIK |
| SEQ ID | DNA scFv | GAAGTGCAGTTGCTGGAGTCAGGCGGAGGACTGGTGCAG |
| NO: 73 | | CCCGGAGGATCGCTTCGCTTGAGCTGCGCAGCCTCAGGCT |
| | | TTACCTTCTCCTCCTACGCCATGTCCTGGGTCAGACAGGC |
| | | TCCCGGGAAGGGACTGGAATGGGTGTCCGCCATTAGCGG |
| | | TTCCGGCGGAAGCACTTACTATGCCGACTCTGTGAAGGG |
| | | CCGCTTCACTATCTCCCGGGACAACTCCAAGAACACCCTG |
| | | TATCTCCAAATGAATTCCCTGAGGGCCGAAGATACCGCG |
| | | GTGTACTACTGCGCTAGACGGGAGTGGTGGTACGACGAT |
| | | TGGTACCTGGACTACTGGGGACAGGGCACTCTCGTGACT |
| | | GTGTCCTCCGGTGGTGGTGGATCGGGGGGTGGTGGTTCG |
| | | GGCGGAGGAGGATCTGGAGGAGGAGGGTCGGACATTCA |
| | | AATGACTCAGTCCCCGTCCTCCCTCTCCGCCTCCGTGGGA |
| | | GATCGCGTCACGATCACGTGCAGGGCCAGCCAGAGCATC |
| | | TCCAGCTACCTGAACTGGTACCAGCAGAAGCCAGGGAAG |
| | | GCACCGAAGCTCCTGATCTACGCCGCTAGCTCGCTGCAGT |
| | | CCGGCGTCCCTTCACGGTTCTCGGGATCGGGCTCAGGCAC |
| | | CGACTTCACCCTGACCATTAGCAGCCTGCAGCCGGAGGA |
| | | CTTCGCGACATACTACTGTCAGCAGTCATACTCCACCCCT |
| | | CTGACCTTCGGCCAAGGGACCAAAGTGGAGATCAAG |
| SEQ ID | Full CAR | EVQLLESGGGLVQPGGSLRLSCAASGFTFSSYAMSWVRQAP |
| NO: 74 | amino acid | GKGLEWVSAISGSGGSTYYADSVKGRFTISRDNSKNTLYLQ |
| | sequence | MNSLRAEDTAVYYCARREWWYDDWYLDYWGQGTLVTVS |
| | | SGGGGSGGGGSGGGGSGGGGSDIQMTQSPSSLSASVGDRVT |
| | | ITCRASQSISSYLNWYQQKPGKAPKLLIYAASSLQSGVPSRF |
| | | SGSGSGTDFTLTISSLQPEDFATYYCQQSYSTPLTFGQGTKV |
| | | EIKTTTPAPRPPTPAPTIASQPLSLRPEACRPAAGGAVHTRGL |
| | | DFACDIYIWAPLAGTCGVLLLSLVITLYCKRGRKKLLYIFKQ |
| | | PFMRPVQTTQEEDGCSCRFPEEEEGGCELRVKFSRSADAPA |
| | | YQQGQNQLYNELNLGRREEYDVLDKRRGRDPEMGGKPRR |
| | | KNPQEGLYNELQKDKMAEAYSEIGMKGERRRGKGHDGLY |
| | | QGLSTATKDTYDALHMQALPPR |
| SEQ ID | Full CAR | GAAGTGCAGTTGCTGGAGTCAGGCGGAGGACTGGTGCAG |
| NO: 75 | DNA | CCCGGAGGATCGCTTCGCTTGAGCTGCGCAGCCTCAGGCT |
| | sequence | TTACCTTCTCCTCCTACGCCATGTCCTGGGTCAGACAGGC |
| | | TCCCGGGAAGGGACTGGAATGGGTGTCCGCCATTAGCGG |
| | | TTCCGGCGGAAGCACTTACTATGCCGACTCTGTGAAGGG |
| | | CCGCTTCACTATCTCCCGGGACAACTCCAAGAACACCCTG |
| | | TATCTCCAAATGAATTCCCTGAGGGCCGAAGATACCGCG |
| | | GTGTACTACTGCGCTAGACGGGAGTGGTGGTACGACGAT |
| | | TGGTACCTGGACTACTGGGGACAGGGCACTCTCGTGACT |
| | | GTGTCCTCCGGTGGTGGTGGATCGGGGGGTGGTGGTTCG |
| | | GGCGGAGGAGGATCTGGAGGAGGAGGGTCGGACATTCA |
| | | AATGACTCAGTCCCCGTCCTCCCTCTCCGCCTCCGTGGGA |
| | | GATCGCGTCACGATCACGTGCAGGGCCAGCCAGAGCATC |
| | | TCCAGCTACCTGAACTGGTACCAGCAGAAGCCAGGGAAG |
| | | GCACCGAAGCTCCTGATCTACGCCGCTAGCTCGCTGCAGT |
| | | CCGGCGTCCCTTCACGGTTCTCGGGATCGGGCTCAGGCAC |
| | | CGACTTCACCCTGACCATTAGCAGCCTGCAGCCGGAGGA |
| | | CTTCGCGACATACTACTGTCAGCAGTCATACTCCACCCCT |
| | | CTGACCTTCGGCCAAGGGACCAAAGTGGAGATCAAGACC |
| | | ACTACCCCAGCACCGAGGCCACCCACCCCGGCTCCTACC |
| | | ATCGCCTCCCAGCCTCTGTCCCTGCGTCCGGAGGCATGTA |
| | | GACCCGCAGCTGGTGGGGCCGTGCATACCCGGGGTCTTG |
| | | ACTTCGCCTGCGATATCTACATTTGGGCCCCTCTGGCTGG |
| | | TACTTGCGGGGTCCTGCTGCTTTCACTCGTGATCACTCTTT |
| | | ACTGTAAGCGCGGTCGGAAGAAGCTGCTGTACATCTTTA |
| | | AGCAACCCTTCATGAGGCCTGTGCAGACTACTCAAGAGG |
| | | AGGACGGCTGTTCATGCCGGTTCCCAGAGGAGGAGGAAG |
| | | GCGGCTGCGAACTGCGCGTGAAATTCAGCCGCAGCGCAG |
| | | ATGCTCCAGCCTACCAGCAGGGGCAGAACCAGCTCTACA |
| | | ACGAACTCAATCTTGGTCGGAGAGAGGAGTACGACGTGC |
| | | TGGACAAGCGGAGAGGACGGGACCCAGAAATGGGCGGG |
| | | AAGCCGCGCAGAAAGAATCCCCAAGAGGGCCTGTACAAC |
| | | GAGCTCCAAAAGGATAAGATGGCAGAAGCCTATAGCGAG |
| | | ATTGGTATGAAAGGGGAACGCAGAAGAGGCAAAGGCCA |
| | | CGACGGACTGTACCAGGGACTCAGCACCGCCACCAAGGA |
| | | CACCTATGACGCTCTTCACATGCAGGCCCTGCCGCCTCGG |
| R1G5 | | |
| SEQ ID | HCDR1 | SYAMS |
| NO: 44 | (Kabat) | |
| SEQ ID | HCDR2 | AISGSGGSTYYADSVKG |
| NO: 45 | (Kabat) | |
| SEQ ID | HCDR3 | REWWGESWLFDY |
| NO: 76 | (Kabat) | |
| SEQ ID | HCDR1 | GFTFSSY |
| NO: 47 | (Chothia) | |
| SEQ ID | HCDR2 | SGSGGS |
| NO: 48 | (Chothia) | |
| SEQ ID | HCDR3 | REWWGESWLFDY |
| NO: 76 | (Chothia) | |
| SEQ ID | HCDR1 | GFTFSSYA |
| NO: 49 | (IMGT) | |
| SEQ ID | HCDR2 | ISGSGGST |
| NO: 50 | (IMGT) | |
| SEQ ID | HCDR3 | ARREWWGESWLFDY |
| NO: 77 | (IMGT) | |
| SEQ ID | VH | EVQLLESGGGLVQPGGSLRLSCAASGFTFSSYAMSWVRQAP |
| NO: 78 | | GKGLEWVSAISGSGGSTYYADSVKGRFTISRDNSKNTLYLQ |
| | | MNSLRAEDTAVYYCARREWWGESWLFDYWGQGTLVTVSS |
| SEQ ID | DNA VH | GAAGTGCAGTTGCTGGAGTCAGGCGGAGGACTGGTGCAG |
| NO: 79 | | CCCGGAGGATCGCTTCGCTTGAGCTGCGCAGCCTCAGGCT |
| | | TTACCTTCTCCTCCTACGCCATGTCCTGGGTCAGACAGGC |
| | | TCCCGGGAAGGGACTGGAATGGGTGTCCGCCATTAGCGG |
| | | TTCCGGCGGAAGCACTTACTATGCCGACTCTGTGAAGGG |
| | | CCGCTTCACTATCTCCCGGGACAACTCCAAGAACACCCTG |
| | | TATCTCCAAATGAATTCCCTGAGGGCCGAAGATACCGCG |
| | | GTGTACTACTGCGCTAGACGGGAGTGGTGGGGAGAAAGC |
| | | TGGCTGTTCGACTACTGGGGACAGGGCACTCTCGTGACTG |
| | | TGTCCTCC |
| SEQ ID | LCDR1 | RASQSISSYLN |
| NO: 54 | (Kabat) | |
| SEQ ID | LCDR2 | AASSLQS |
| NO: 55 | (Kabat) | |
| SEQ ID | LCDR3 | QQSYSTPLT |
| NO: 56 | (Kabat) | |
| SEQ ID | LCDR1 | SQSISSY |
| NO: 57 | (Chothia) | |
| SEQ ID | LCDR2 | AAS |
| NO: 58 | (Chothia) | |
| SEQ ID | LCDR3 | SYSTPL |
| NO: 59 | (Chothia) | |
| SEQ ID | LCDR1 | QSISSY |
| NO: 60 | (IMGT) | |
| SEQ ID | LCDR2 | AAS |
| NO: 58 | (IMGT) | |
| SEQ ID | LCDR3 | QQSYSTPLT |
| NO: 56 | (IMGT) | |
| SEQ ID | VL | DIQMTQSPSSLSASVGDRVTITCRASQSISSYLNWYQQKPGK |
| NO: 61 | | APKLLIYAASSLQSGVPSRFSGSGSGTDFTLTISSLQPEDFAT |
| | | YYCQQSYSTPLTFGQGTKVEIK |
| SEQ ID | DNA VL | GACATTCAAATGACTCAGTCCCCGTCCTCCCTCTCCGCCT |
| NO: 62 | | CCGTGGGAGATCGCGTCACGATCACGTGCAGGGCCAGCC |
| | | AGAGCATCTCCAGCTACCTGAACTGGTACCAGCAGAAGC |
| | | CAGGGAAGGCACCGAAGCTCCTGATCTACGCCGCTAGCT |
| | | CGCTGCAGTCCGGCGTCCCTTCACGGTTCTCGGGATCGGG |
| | | CTCAGGCACCGACTTCACCCTGACCATTAGCAGCCTGCAG |
| | | CCGGAGGACTTCGCGACATACTACTGTCAGCAGTCATACT |
| | | CCACCCCTCTGACCTTCGGCCAAGGGACCAAAGTGGAGA |
| | | TCAAG |
| SEQ ID | Linker | GGGGSGGGGSGGGGSGGGGS |
| NO: 63 | | |
| SEQ ID | scFv (VH- | EVQLLESGGGLVQPGGSLRLSCAASGFTFSSYAMSWVRQAP |
| NO: 80 | linker-VL) | GKGLEWVSAISGSGGSTYYADSVKGRFTISRDNSKNTLYLQ |
| | | MNSLRAEDTAVYYCARREWWGESWLFDYWGQGTLVTVSS |
| | | GGGGSGGGGSGGGGSGGGGSDIQMTQSPSSLSASVGDRVTI |
| | | TCRASQSISSYLNWYQQKPGKAPKLLIYAASSLQSGVPSRFS |
| | | GSGSGTDFTLTISSLQPEDFATYYCQQSYSTPLTFGQGTKVEI |
| | | K |
| SEQ ID | DNA scFv | GAAGTGCAGTTGCTGGAGTCAGGCGGAGGACTGGTGCAG |
| NO: 81 | | CCCGGAGGATCGCTTCGCTTGAGCTGCGCAGCCTCAGGCT |
| | | TTACCTTCTCCTCCTACGCCATGTCCTGGGTCAGACAGGC |
| | | TCCCGGGAAGGGACTGGAATGGGTGTCCGCCATTAGCGG |
| | | TTCCGGCGGAAGCACTTACTATGCCGACTCTGTGAAGGG |
| | | CCGCTTCACTATCTCCCGGGACAACTCCAAGAACACCCTG |
| | | TATCTCCAAATGAATTCCCTGAGGGCCGAAGATACCGCG |
| | | GTGTACTACTGCGCTAGACGGGAGTGGTGGGGAGAAAGC |
| | | TGGCTGTTCGACTACTGGGGACAGGGCACTCTCGTGACTG |
| | | TGTCCTCCGGTGGTGGTGGATCGGGGGGTGGTGGTTCGG |
| | | GCGGAGGAGGATCTGGAGGAGGAGGGTCGGACATTCAA |
| | | ATGACTCAGTCCCCGTCCTCCCTCTCCGCCTCCGTGGGAG |
| | | ATCGCGTCACGATCACGTGCAGGGCCAGCCAGAGCATCT |
| | | CCAGCTACCTGAACTGGTACCAGCAGAAGCCAGGGAAGG |
| | | CACCGAAGCTCCTGATCTACGCCGCTAGCTCGCTGCAGTC |
| | | CGGCGTCCCTTCACGGTTCTCGGGATCGGGCTCAGGCACC |
| | | GACTTCACCCTGACCATTAGCAGCCTGCAGCCGGAGGAC |
| | | TTCGCGACATACTACTGTCAGCAGTCATACTCCACCCCTC |
| | | TGACCTTCGGCCAAGGGACCAAAGTGGAGATCAAG |
| SEQ ID | Full CAR | EVQLLESGGGLVQPGGSLRLSCAASGFTFSSYAMSWVRQAP |
| NO: 82 | amino acid | GKGLEWVSAISGSGGSTYYADSVKGRFTISRDNSKNTLYLQ |
| | sequence | MNSLRAEDTAVYYCARREWWGESWLFDYWGQGTLVTVSS |
| | | GGGGSGGGGSGGGGSGGGGSDIQMTQSPSSLSASVGDRVTI |
| | | TCRASQSISSYLNWYQQKPGKAPKLLIYAASSLQSGVPSRFS |
| | | GSGSGTDFTLTISSLQPEDFATYYCQQSYSTPLTFGQGTKVEI |
| | | KTTTPAPRPPTPAPTIASQPLSLRPEACRPAAGGAVHTRGLD |
| | | FACDIYIWAPLAGTCGVLLLSLVITLYCKRGRKKLLYIFKQP |
| | | FMRPVQTTQEEDGCSCRFPEEEEGGCELRVKFSRSADAPAY |
| | | QQGQNQLYNELNLGRREEYDVLDKRRGRDPEMGGKPRRK |
| | | NPQEGLYNELQKDKMAEAYSEIGMKGERRRGKGHDGLYQ |
| | | GLSTATKDTYDALHMQALPPR |
| SEQ ID | Full CAR | GAAGTGCAGTTGCTGGAGTCAGGCGGAGGACTGGTGCAG |
| NO: 83 | DNA | CCCGGAGGATCGCTTCGCTTGAGCTGCGCAGCCTCAGGCT |
| | sequence | TTACCTTCTCCTCCTACGCCATGTCCTGGGTCAGACAGGC |
| | | TCCCGGGAAGGGACTGGAATGGGTGTCCGCCATTAGCGG |
| | | TTCCGGCGGAAGCACTTACTATGCCGACTCTGTGAAGGG |
| | | CCGCTTCACTATCTCCCGGGACAACTCCAAGAACACCCTG |
| | | TATCTCCAAATGAATTCCCTGAGGGCCGAAGATACCGCG |
| | | GTGTACTACTGCGCTAGACGGGAGTGGTGGGGAGAAAGC |
| | | TGGCTGTTCGACTACTGGGGACAGGGCACTCTCGTGACTG |
| | | TGTCCTCCGGTGGTGGTGGATCGGGGGGTGGTGGTTCGG |
| | | GCGGAGGAGGATCTGGAGGAGGAGGGTCGGACATTCAA |
| | | ATGACTCAGTCCCCGTCCTCCCTCTCCGCCTCCGTGGGAG |
| | | ATCGCGTCACGATCACGTGCAGGGCCAGCCAGAGCATCT |
| | | CCAGCTACCTGAACTGGTACCAGCAGAAGCCAGGGAAGG |
| | | CACCGAAGCTCCTGATCTACGCCGCTAGCTCGCTGCAGTC |
| | | CGGCGTCCCTTCACGGTTCTCGGGATCGGGCTCAGGCACC |
| | | GACTTCACCCTGACCATTAGCAGCCTGCAGCCGGAGGAC |
| | | TTCGCGACATACTACTGTCAGCAGTCATACTCCACCCCTC |
| | | TGACCTTCGGCCAAGGGACCAAAGTGGAGATCAAGACCA |
| | | CTACCCCAGCACCGAGGCCACCCACCCCGGCTCCTACCA |
| | | TCGCCTCCCAGCCTCTGTCCCTGCGTCCGGAGGCATGTAG |
| | | ACCCGCAGCTGGTGGGGCCGTGCATACCCGGGGTCTTGA |
| | | CTTCGCCTGCGATATCTACATTTGGGCCCCTCTGGCTGGT |
| | | ACTTGCGGGGTCCTGCTGCTTTCACTCGTGATCACTCTTT |
| | | ACTGTAAGCGCGGTCGGAAGAAGCTGCTGTACATCTTTA |
| | | AGCAACCCTTCATGAGGCCTGTGCAGACTACTCAAGAGG |
| | | AGGACGGCTGTTCATGCCGGTTCCCAGAGGAGGAGGAAG |
| | | GCGGCTGCGAACTGCGCGTGAAATTCAGCCGCAGCGCAG |
| | | ATGCTCCAGCCTACCAGCAGGGGCAGAACCAGCTCTACA |
| | | ACGAACTCAATCTTGGTCGGAGAGAGGAGTACGACGTGC |
| | | TGGACAAGCGGAGAGGACGGGACCCAGAAATGGGCGGG |
| | | AAGCCGCGCAGAAAGAATCCCCAAGAGGGCCTGTACAAC |
| | | GAGCTCCAAAAGGATAAGATGGCAGAAGCCTATAGCGAG |
| | | ATTGGTATGAAAGGGGAACGCAGAAGAGGCAAAGGCCA |
| | | CGACGGACTGTACCAGGGACTCAGCACCGCCACCAAGGA |
| | | CACCTATGACGCTCTTCACATGCAGGCCCTGCCGCCTCGG |

TABLE 3
Kabat CDRs of exemplary PALLAS-derived anti-BCMA molecules

| Kabat | HCDR1 | HCDR2 | HCDR3 | LCDR1 | LCDR2 | LCDR3 |
|---|---|---|---|---|---|---|
| R1B6 | SYAMS | AISGSGGSTY | REWVPYDV | RASQSISS | AASSL | QQSYST |
| | (SEQ ID | YADSVKG | SWYFDY | YLN (SEQ | QS | PLT |
| | NO: 44) | (SEQ ID NO: | (SEQ ID NO: | ID NO: 54) | (SEQ | (SEQ ID |
| | | 45) | 46) | | ID NO: | NO: 56) |
| | | | | | 55 | |
| R1F2 | SYAMS | AISGSGGSTY | REWWYDD | RASQSISS | AASSL | QQSYST |
| | (SEQ ID | YADSVKG | WYLDY | YLN (SEQ | QS | PLT |
| | NO: 44) | (SEQ ID NO: | (SEQ ID NO: | ID NO: 54) | (SEQ | (SEQ ID |
| | | 45) | 68) | | ID NO: | NO: 56) |
| | | | | | 55) | |
| R1G5 | SYAMS | AISGSGGSTY | REWWGES | RASQSISS | AASSL | QQSYST |
| | (SEQ ID | YADSVKG | WLFDY | YLN (SEQ | QS | PLT |
| | NO: 44) | (SEQ ID NO: | (SEQ ID NO: | ID NO: 54) | (SEQ | (SEQ ID |
| | | 45) | 76) | | ID NO: | NO: 56) |
| | | | | | 55) | |
| Consensus | SYAMS | AISGSGGSTY | REWX₁X₂X₃ | RASQSISS | AASSL | QQSYST |
| | (SEQ ID | YADSVKG | X₄X₅X₆WX₇ | YLN (SEQ | QS | PLT |
| | NO: 44) | (SEQ ID NO: | X₈DY, | ID NO: 54) | (SEQ | (SEQ ID |
| | | 45) | wherein X₁ is | | ID NO: | NO: 56) |
| | | | absent or V; | | 55) | |
| | | | X₂ is absent | | | |
| | | | or P; X₃ is W | | | |
| | | | or Y; X₄ is G, | | | |
| | | | Y, or D; X₅ is | | | |
| | | | E, D, or V; X₆ | | | |
| | | | is S or D; X₇ | | | |
| | | | is L or Y; and | | | |
| | | | X₈ is F or L | | | |
| | | | (SEQ ID NO: | | | |
| | | | 84) | | | |

TABLE 4
Chothia CDRs of exemplary PALLAS-derived anti-BCMA molecules

| Chothia | HCDR1 | HCDR2 | HCDR3 | LCDR1 | LCDR2 | LCDR3 |
|---|---|---|---|---|---|---|
| R1B6 | GFTFSSY | SGSGGS | REWVPYDV | SQSISSY | AAS | SYSTPL |
| | (SEQ ID | (SEQ ID NO: | SWYFDY | (SEQ ID | (SEQ | (SEQ ID |
| | NO: 47) | 48) | (SEQ ID NO: | NO: 57) | ID NO: | NO: 59) |
| | | | 46) | | 58) | |
| R1F2 | GFTFSSY | SGSGGS | REWWYDD | SQSISSY | AAS | SYSTPL |
| | (SEQ ID | (SEQ ID NO: | WYLDY | (SEQ ID | (SEQ | (SEQ ID |
| | NO: 47) | 48) | (SEQ ID NO: | NO: 57) | ID NO: | NO: 59) |
| | | | 68) | | 58) | |
| R1G5 | GFTFSSY | SGSGGS | REWWGES | SQSISSY | AAS | SYSTPL |
| | (SEQ ID | (SEQ ID NO: | WLFDY | (SEQ ID | (SEQ | (SEQ ID |
| | NO: 47) | 48) | (SEQ ID NO: | NO: 57) | ID NO: | NO: 59) |
| | | | 76) | | 58) | |
| Consensus | GFTFSSY | SGSGGS | REWX₁X₂X₃ | SQSISSY | AAS | SYSTPL |
| | (SEQ ID | (SEQ ID NO: | X₄X₅X₆WX₇ | (SEQ ID | (SEQ | (SEQ ID |
| | NO: 47) | 48) | X₈DY, | NO: 57) | ID NO: | NO: 59) |
| | | | wherein X₁ is | | 58) | |
| | | | absent or V; | | | |
| | | | X₂ is absent | | | |
| | | | or P; X₃ is W | | | |
| | | | or Y; X₄ is G, | | | |
| | | | Y, or D; X₅ is | | | |
| | | | E, D, or V; X₆ | | | |
| | | | is S or D; X₇ | | | |
| | | | is L or Y; and | | | |
| | | | X₈ is F or L | | | |
| | | | (SEQ ID NO: | | | |
| | | | 84) | | | |

TABLE 5
IMGT CDRs of exemplary PALLAS-derived anti-BCMA molecules

| IMGT | HCDR1 | HCDR2 | HCDR3 | LCDR1 | LCDR2 | LCDR3 |
|---|---|---|---|---|---|---|
| R1B6 | GFTFSSY | ISGSGGST | ARREWVPY | QSISSY | AAS | QQSYST |
| | A (SEQ ID | (SEQ ID NO: | DVSWYFDY | (SEQ ID | (SEQ | PLT |
| | NO: 49) | 50) | (SEQ ID NO: | NO: 60) | ID NO: | (SEQ ID |
| | | | 51) | | 58) | NO: 56) |
| R1F2 | GFTFSSY | ISGSGGST | ARREWWY | QSISSY | AAS | QQSYST |
| | A (SEQ ID | (SEQ ID NO: | DDWYLDY | (SEQ ID | (SEQ | PLT |
| | NO: 49) | 50) | (SEQ ID NO: | NO: 60) | ID NO: | (SEQ ID |
| | | | 69) | | 58) | NO: 56) |
| R1G5 | GFTFSSY | ISGSGGST | ARREWWG | QSISSY | AAS | QQSYST |
| | A (SEQ ID | (SEQ ID NO: | ESWLFDY | (SEQ ID | (SEQ | PLT |
| | NO: 49) | 50) | (SEQ ID NO: | NO: 60) | ID NO: | (SEQ ID |
| | | | 77) | | 58) | NO: 56) |
| Consensus | GFTFSSY | ISGSGGST | ARREWX₁X₂ | QSISSY | AAS | QQSYST |
| | A (SEQ ID | (SEQ ID NO: | X₃X₄X₅X₆W | (SEQ ID | (SEQ | PLT |
| | NO: 49) | 50) | X₇X₈DY, | NO: 60) | ID NO: | (SEQ ID |
| | | | wherein X₁ is | | 58) | NO: 56) |
| | | | absent or V; | | | |
| | | | X₂ is absent | | | |
| | | | or P; X₃ is W | | | |
| | | | or Y; X₄ is G, | | | |
| | | | Y, or D; X₅ is | | | |
| | | | E, D, or V; X₆ | | | |
| | | | is S or D; X₇ | | | |
| | | | is L or Y; and | | | |
| | | | X₈ is F or L | | | |
| | | | (SEQ ID NO: | | | |
| | | | 85) | | | |

TABLE 6
Amino acid and nucleic acid sequences of exemplary B cell-derived anti-BCMA
molecules

| SEQ | Name/ | |
|---|---|---|
| ID NO | Description | Sequence |
| PI61 | | |
| SEQ ID | HCDR1 | SYGMH |
| NO: 86 | (Kabat) | |
| SEQ ID | HCDR2 | VISYDGSNKYYADSVKG |
| NO: 87 | (Kabat) | |
| SEQ ID | HCDR3 | SGYALHDDYYGLDV |
| NO: 88 | (Kabat) | |
| SEQ ID | HCDR1 | GFTFSSY |
| NO: 47 | (Chothia) | |
| SEQ ID | HCDR2 | SYDGSN |
| NO: 89 | (Chothia) | |
| SEQ ID | HCDR3 | SGYALHDDYYGLDV |
| NO: 88 | (Chothia) | |
| SEQ ID | HCDR1 | GFTFSSYG |
| NO: 90 | (IMGT) | |
| SEQ ID | HCDR2 | ISYDGSNK |
| NO: 91 | (IMGT) | |
| SEQ ID | HCDR3 | GGSGYALHDDYYGLDV |
| NO: 92 | (IMGT) | |
| SEQ ID | VH | QVQLQESGGGVVQPGRSLRLSCAASGFTFSSYGMHWVRQAP |
| NO: 93 | | GKGLEWVAVISYDGSNKYYADSVKGRFTISRDNSKNTLYLQ |
| | | MNSLRAEDTAVYYCGGSGYALHDDYYGLDVWGQGTLVTV |
| | | SS |
| SEQ ID | DNA VH | CAAGTGCAGCTGCAGGAATCCGGTGGCGGAGTCGTGCAG |
| NO: 94 | | CCTGGAAGGAGCCTGAGACTCTCATGCGCCGCGTCAGGGT |
| | | TCACCTTTTCCTCCTACGGGATGCATTGGGTCAGACAGGC |
| | | CCCCGGAAAGGGACTCGAATGGGTGGCTGTGATCAGCTAC |
| | | GACGGCTCCAACAAGTACTACGCCGACTCCGTGAAAGGCC |
| | | GGTTCACTATCTCCCGGGACAACTCCAAGAACACGCTGTA |
| | | TCTGCAAATGAATTCACTGCGCGCGGAGGATACCGCTGTG |
| | | TACTACTGCGGTGGCTCCGGTTACGCCCTGCACGATGACT |
| | | ATTACGGCCTTGACGTCTGGGGCCAGGGAACCCTCGTGAC |
| | | TGTGTCCAGC |
| SEQ ID | LCDR1 | TGTSSDVGGYNYVS |
| NO: 95 | (Kabat) | |
| SEQ ID | LCDR2 | DVSNRPS |
| NO: 96 | (Kabat) | |
| SEQ ID | LCDR3 | SSYTSSSTLYV |
| NO: 97 | (Kabat) | |
| SEQ ID | LCDR1 | TSSDVGGYNY |
| NO: 98 | (Chothia) | |
| SEQ ID | LCDR2 | DVS |
| NO: 99 | (Chothia) | |
| SEQ ID | LCDR3 | YTSSSTLY |
| NO: | (Chothia) | |
| 100 | | |
| SEQ ID | LCDR1 | SSDVGGYNY |
| NO: | (IMGT) | |
| 101 | | |
| SEQ ID | LCDR2 | DVS |
| NO: 99 | (IMGT) | |
| SEQ ID | LCDR3 | SSYTSSSTLYV |
| NO: 97 | (IMGT) | |
| SEQ ID | VL | QSALTQPASVSGSPGQSITISCTGTSSDVGGYNYVSWYQQHP |
| NO: | | GKAPKLMIYDVSNRPSGVSNRFSGSKSGNTASLTISGLQAED |
| 102 | | EADYYCSSYTSSSTLYVFGSGTKVTVL |
| SEQ ID | DNA VL | CAGAGCGCACTGACTCAGCCGGCATCCGTGTCCGGTAGCC |
| NO: | | CCGGACAGTCGATTACCATCTCCTGTACCGGCACCTCCTC |
| 103 | | CGACGTGGGAGGGTACAACTACGTGTCGTGGTACCAGCA |
| | | GCACCCAGGAAAGGCCCCTAAGTTGATGATCTACGATGTG |
| | | TCAAACCGCCCGTCTGGAGTCTCCAACCGGTTCTCCGGCT |
| | | CCAAGTCCGGCAACACCGCCAGCCTGACCATTAGCGGGCT |
| | | GCAAGCCGAGGATGAGGCCGACTACTACTGCTCGAGCTAC |
| | | ACATCCTCGAGCACCCTCTACGTGTTCGGCTCGGGGACTA |
| | | AGGTCACCGTGCTG |
| SEQ ID | Linker | GGGGSGGGGSGGGGS |
| NO: | | |
| 104 | | |
| SEQ ID | scFv (VH- | QVQLQESGGGVVQPGRSLRLSCAASGFTFSSYGMHWVRQAP |
| NO: | linker-VL) | GKGLEWVAVISYDGSNKYYADSVKGRFTISRDNSKNTLYLQ |
| 105 | | MNSLRAEDTAVYYCGGSGYALHDDYYGLDVWGQGTLVTV |
| | | SSGGGGSGGGGSGGGGSQSALTQPASVSGSPGQSITISCTGTS |
| | | SDVGGYNYVSWYQQHPGKAPKLMIYDVSNRPSGVSNRFSGS |
| | | KSGNTASLTISGLQAEDEADYYCSSYTSSSTLYVFGSGTKVT |
| | | VL |
| SEQ ID | DNA scFv | CAAGTGCAGCTGCAGGAATCCGGTGGCGGAGTCGTGCAG |
| NO: | | CCTGGAAGGAGCCTGAGACTCTCATGCGCCGCGTCAGGGT |
| 106 | | TCACCTTTTCCTCCTACGGGATGCATTGGGTCAGACAGGC |
| | | CCCCGGAAAGGGACTCGAATGGGTGGCTGTGATCAGCTAC |
| | | GACGGCTCCAACAAGTACTACGCCGACTCCGTGAAAGGCC |
| | | GGTTCACTATCTCCCGGGACAACTCCAAGAACACGCTGTA |
| | | TCTGCAAATGAATTCACTGCGCGCGGAGGATACCGCTGTG |
| | | TACTACTGCGGTGGCTCCGGTTACGCCCTGCACGATGACT |
| | | ATTACGGCCTTGACGTCTGGGGCCAGGGAACCCTCGTGAC |
| | | TGTGTCCAGCGGTGGAGGAGGTTCGGGCGGAGGAGGATC |
| | | AGGAGGGGGTGGATCGCAGAGCGCACTGACTCAGCCGGC |
| | | ATCCGTGTCCGGTAGCCCCGGACAGTCGATTACCATCTCC |
| | | TGTACCGGCACCTCCTCCGACGTGGGAGGGTACAACTACG |
| | | TGTCGTGGTACCAGCAGCACCCAGGAAAGGCCCCTAAGTT |
| | | GATGATCTACGATGTGTCAAACCGCCCGTCTGGAGTCTCC |
| | | AACCGGTTCTCCGGCTCCAAGTCCGGCAACACCGCCAGCC |
| | | TGACCATTAGCGGGCTGCAAGCCGAGGATGAGGCCGACT |
| | | ACTACTGCTCGAGCTACACATCCTCGAGCACCCTCTACGT |
| | | GTTCGGCTCGGGGACTAAGGTCACCGTGCTG |
| SEQ ID | Full CAR | QVQLQESGGGVVQPGRSLRLSCAASGFTFSSYGMHWVRQAP |
| NO: | amino acid | GKGLEWVAVISYDGSNKYYADSVKGRFTISRDNSKNTLYLQ |
| 107 | sequence | MNSLRAEDTAVYYCGGSGYALHDDYYGLDVWGQGTLVTV |
| | | SSGGGGSGGGGSGGGGSQSALTQPASVSGSPGQSITISCTGTS |
| | | SDVGGYNYVSWYQQHPGKAPKLMIYDVSNRPSGVSNRFSGS |
| | | KSGNTASLTISGLQAEDEADYYCSSYTSSSTLYVFGSGTKVT |
| | | VLTTTPAPRPPTPAPTIASQPLSLRPEACRPAAGGAVHTRGLD |
| | | FACDIYIWAPLAGTCGVLLLSLVITLYCKRGRKKLLYIFKQPF |
| | | MRPVQTTQEEDGCSCRFPEEEEGGCELRVKFSRSADAPAYQ |
| | | QGQNQLYNELNLGRREEYDVLDKRRGRDPEMGGKPRRKNP |
| | | QEGLYNELQKDKMAEAYSEIGMKGERRRGKGHDGLYQGLS |
| | | TATKDTYDALHMQALPPR |
| SEQ ID | Full CAR | CAAGTGCAGCTGCAGGAATCCGGTGGCGGAGTCGTGCAG |
| NO: | DNA | CCTGGAAGGAGCCTGAGACTCTCATGCGCCGCGTCAGGGT |
| 108 | sequence | TCACCTTTTCCTCCTACGGGATGCATTGGGTCAGACAGGC |
| | | CCCCGGAAAGGGACTCGAATGGGTGGCTGTGATCAGCTAC |
| | | GACGGCTCCAACAAGTACTACGCCGACTCCGTGAAAGGCC |
| | | GGTTCACTATCTCCCGGGACAACTCCAAGAACACGCTGTA |
| | | TCTGCAAATGAATTCACTGCGCGCGGAGGATACCGCTGTG |
| | | TACTACTGCGGTGGCTCCGGTTACGCCCTGCACGATGACT |
| | | ATTACGGCCTTGACGTCTGGGGCCAGGGAACCCTCGTGAC |
| | | TGTGTCCAGCGGTGGAGGAGGTTCGGGCGGAGGAGGATC |
| | | AGGAGGGGGTGGATCGCAGAGCGCACTGACTCAGCCGGC |
| | | ATCCGTGTCCGGTAGCCCCGGACAGTCGATTACCATCTCC |
| | | TGTACCGGCACCTCCTCCGACGTGGGAGGGTACAACTACG |
| | | TGTCGTGGTACCAGCAGCACCCAGGAAAGGCCCCTAAGTT |
| | | GATGATCTACGATGTGTCAAACCGCCCGTCTGGAGTCTCC |
| | | AACCGGTTCTCCGGCTCCAAGTCCGGCAACACCGCCAGCC |
| | | TGACCATTAGCGGGCTGCAAGCCGAGGATGAGGCCGACT |
| | | ACTACTGCTCGAGCTACACATCCTCGAGCACCCTCTACGT |
| | | GTTCGGCTCGGGGACTAAGGTCACCGTGCTGACCACTACC |
| | | CCAGCACCGAGGCCACCCACCCCGGCTCCTACCATCGCCT |
| | | CCCAGCCTCTGTCCCTGCGTCCGGAGGCATGTAGACCCGC |
| | | AGCTGGTGGGGCCGTGCATACCCGGGGTCTTGACTTCGCC |
| | | TGCGATATCTACATTTGGGCCCCTCTGGCTGGTACTTGCGG |
| | | GGTCCTGCTGCTTTCACTCGTGATCACTCTTTACTGTAAGC |
| | | GCGGTCGGAAGAAGCTGCTGTACATCTTTAAGCAACCCTT |
| | | CATGAGGCCTGTGCAGACTACTCAAGAGGAGGACGGCTG |
| | | TTCATGCCGGTTCCCAGAGGAGGAGGAAGGCGGCTGCGA |
| | | ACTGCGCGTGAAATTCAGCCGCAGCGCAGATGCTCCAGCC |
| | | TACCAGCAGGGGCAGAACCAGCTCTACAACGAACTCAAT |
| | | CTTGGTCGGAGAGAGGAGTACGACGTGCTGGACAAGCGG |
| | | AGAGGACGGGACCCAGAAATGGGCGGGAAGCCGCGCAGA |
| | | AAGAATCCCCAAGAGGGCCTGTACAACGAGCTCCAAAAG |
| | | GATAAGATGGCAGAAGCCTATAGCGAGATTGGTATGAAA |
| | | GGGGAACGCAGAAGAGGCAAAGGCCACGACGGACTGTAC |
| | | CAGGGACTCAGCACCGCCACcaaggacacctatgacgctcttcacatgcagg |
| | | ccctgccgcctcgg |
| B61-02 | | |
| SEQ ID | HCDR1 | SYGMH |
| NO: 86 | (Kabat) | |
| SEQ ID | HCDR2 | VISYKGSNKYYADSVKG |
| NO: | (Kabat) | |
| 109 | | |
| SEQ ID | HCDR3 | SGYALHDDYYGLDV |
| NO: 88 | (Kabat) | |
| SEQ ID | HCDR1 | GFTFSSY |
| NO: 47 | (Chothia) | |
| SEQ ID | HCDR2 | SYKGSN |
| NO: | (Chothia) | |
| 110 | | |
| SEQ ID | HCDR3 | SGYALHDDYYGLDV |
| NO: 88 | (Chothia) | |
| SEQ ID | HCDR1 | GFTFSSYG |
| NO: 90 | (IMGT) | |
| SEQ ID | HCDR2 | ISYKGSNK |
| NO: | (IMGT) | |
| 111 | | |
| SEQ ID | HCDR3 | GGSGYALHDDYYGLDV |
| NO: 92 | (IMGT) | |
| SEQ ID | VH | QVQLVESGGGVVQPGRSLRLSCAASGFTFSSYGMHWVRQAP |
| NO: | | GKGLEWVAVISYKGSNKYYADSVKGRFTISRDNSKNTLYLQ |
| 112 | | MNSLRAEDTAVYYCGGSGYALHDDYYGLDVWGQGTLVTV |
| | | SS |
| SEQ ID | DNA VH | CAAGTGCAGCTTGTCGAATCGGGAGGCGGAGTGGTGCAG |
| NO: | | CCTGGACGATCGCTCCGGCTCTCATGTGCCGCGAGCGGAT |
| 113 | | TCACCTTCTCGAGCTACGGCATGCACTGGGTCAGACAAGC |
| | | CCCAGGAAAGGGCCTGGAATGGGTGGCTGTCATCTCGTAC |
| | | AAGGGCTCAAACAAGTACTACGCCGACTCCGTGAAGGGC |
| | | CGGTTCACCATCTCCCGCGATAACTCCAAGAATACCCTCT |
| | | ATCTGCAAATGAACAGCCTGAGGGCCGAGGATACTGCAG |
| | | TGTACTACTGCGGGGGTTCAGGCTACGCGCTGCACGACGA |
| | | CTACTACGGATTGGACGTCTGGGGCCAAGGAACTCTTGTG |
| | | ACCGTGTCCTCT |
| SEQ ID | LCDR1 | TGTSSDVGGYNYVS |
| NO: 95 | (Kabat) | |
| SEQ ID | LCDR2 | EVSNRLR |
| NO: | (Kabat) | |
| 114 | | |
| SEQ ID | LCDR3 | SSYTSSSALYV |
| NO: | (Kabat) | |
| 115 | | |
| SEQ ID | LCDR1 | TSSDVGGYNY |
| NO: 98 | (Chothia) | |
| SEQ ID | LCDR2 | EVS |
| NO: | (Chothia) | |
| 116 | | |
| SEQ ID | LCDR3 | YTSSSALY |
| NO: | (Chothia) | |
| 117 | | |
| SEQ ID | LCDR1 | SSDVGGYNY |
| NO: | (IMGT) | |
| 101 | | |
| SEQ ID | LCDR2 | EVS |
| NO: | (IMGT) | |
| 116 | | |
| SEQ ID | LCDR3 | SSYTSSSALYV |
| NO: | (IMGT) | |
| 115 | | |
| SEQ ID | VL | QSALTQPASVSGSPGQSITISCTGTSSDVGGYNYVSWYQQHP |
| NO: | | GKAPKLMIYEVSNRLRGVSNRFSGSKSGNTASLTISGLQAED |
| 118 | | EADYYCSSYTSSSALYVFGSGTKVTVL |
| SEQ ID | DNA VL | CAGAGCGCGCTGACTCAGCCTGCCTCCGTGAGCGGTTCGC |
| NO: | | CGGGACAGTCCATTACCATTTCGTGCACCGGGACCTCCTC |
| 119 | | CGACGTGGGAGGCTACAACTACGTGTCCTGGTACCAGCAG |
| | | CATCCCGGAAAGGCCCCGAAGCTGATGATCTACGAAGTGT |
| | | CGAACAGACTGCGGGGAGTCTCCAACCGCTTTTCCGGGTC |
| | | CAAGTCCGGCAACACCGCCAGCCTGACCATCAGCGGGCTC |
| | | CAGGCAGAAGATGAGGCTGACTATTACTGCTCCTCCTACA |
| | | CGTCAAGCTCCGCCCTCTACGTGTTCGGGTCCGGGACCAA |
| | | AGTCACTGTGCTG |
| SEQ ID | Linker | GGGGSGGGGSGGGGSGGGGS |
| NO: 63 | | |
| SEQ ID | scFv (VH- | QVQLVESGGGVVQPGRSLRLSCAASGFTFSSYGMHWVRQAP |
| NO: | linker-VL) | GKGLEWVAVISYKGSNKYYADSVKGRFTISRDNSKNTLYLQ |
| 120 | | MNSLRAEDTAVYYCGGSGYALHDDYYGLDVWGQGTLVTV |
| | | SSGGGGSGGGGSGGGGSGGGGSQSALTQPASVSGSPGQSITI |
| | | SCTGTSSDVGGYNYVSWYQQHPGKAPKLMIYEVSNRLRGVS |
| | | NRFSGSKSGNTASLTISGLQAEDEADYYCSSYTSSSALYVFGS |
| | | GTKVTVL |
| SEQ ID | DNA scFv | CAAGTGCAGCTTGTCGAATCGGGAGGCGGAGTGGTGCAG |
| NO: | | CCTGGACGATCGCTCCGGCTCTCATGTGCCGCGAGCGGAT |
| 121 | | TCACCTTCTCGAGCTACGGCATGCACTGGGTCAGACAAGC |
| | | CCCAGGAAAGGGCCTGGAATGGGTGGCTGTCATCTCGTAC |
| | | AAGGGCTCAAACAAGTACTACGCCGACTCCGTGAAGGGC |
| | | CGGTTCACCATCTCCCGCGATAACTCCAAGAATACCCTCT |
| | | ATCTGCAAATGAACAGCCTGAGGGCCGAGGATACTGCAG |
| | | TGTACTACTGCGGGGGTTCAGGCTACGCGCTGCACGACGA |
| | | CTACTACGGATTGGACGTCTGGGGCCAAGGAACTCTTGTG |
| | | ACCGTGTCCTCTGGTGGAGGCGGATCAGGGGGTGGCGGAT |
| | | CTGGGGGTGGTGGTTCCGGGGGAGGAGGATCGCAGAGCG |
| | | CGCTGACTCAGCCTGCCTCCGTGAGCGGTTCGCCGGGACA |
| | | GTCCATTACCATTTCGTGCACCGGGACCTCCTCCGACGTG |
| | | GGAGGCTACAACTACGTGTCCTGGTACCAGCAGCATCCCG |
| | | GAAAGGCCCCGAAGCTGATGATCTACGAAGTGTCGAACA |
| | | GACTGCGGGGAGTCTCCAACCGCTTTTCCGGGTCCAAGTC |
| | | CGGCAACACCGCCAGCCTGACCATCAGCGGGCTCCAGGC |
| | | AGAAGATGAGGCTGACTATTACTGCTCCTCCTACACGTCA |
| | | AGCTCCGCCCTCTACGTGTTCGGGTCCGGGACCAAAGTCA |
| | | CTGTGCTG |
| SEQ ID | Full CAR | QVQLVESGGGVVQPGRSLRLSCAASGFTFSSYGMHWVRQAP |
| NO: | amino acid | GKGLEWVAVISYKGSNKYYADSVKGRFTISRDNSKNTLYLQ |
| 122 | sequence | MNSLRAEDTAVYYCGGSGYALHDDYYGLDVWGQGTLVTV |
| | | SSGGGGSGGGGSGGGGSGGGGSQSALTQPASVSGSPGQSITI |
| | | SCTGTSSDVGGYNYVSWYQQHPGKAPKLMIYEVSNRLRGVS |
| | | NRFSGSKSGNTASLTISGLQAEDEADYYCSSYTSSSALYVFGS |
| | | GTKVTVLTTTPAPRPPTPAPTIASQPLSLRPEACRPAAGGAVH |
| | | TRGLDFACDIYIWAPLAGTCGVLLLSLVITLYCKRGRKKLLYI |
| | | FKQPFMRPVQTTQEEDGCSCRFPEEEEGGCELRVKFSRSADA |
| | | PAYQQGQNQLYNELNLGRREEYDVLDKRRGRDPEMGGKPR |
| | | RKNPQEGLYNELQKDKMAEAYSEIGMKGERRRGKGHDGLY |
| | | QGLSTATKDTYDALHMQALPPR |
| SEQ ID | Full CAR | CAAGTGCAGCTTGTCGAATCGGGAGGCGGAGTGGTGCAG |
| NO: | DNA | CCTGGACGATCGCTCCGGCTCTCATGTGCCGCGAGCGGAT |
| 123 | sequence | TCACCTTCTCGAGCTACGGCATGCACTGGGTCAGACAAGC |
| | | CCCAGGAAAGGGCCTGGAATGGGTGGCTGTCATCTCGTAC |
| | | AAGGGCTCAAACAAGTACTACGCCGACTCCGTGAAGGGC |
| | | CGGTTCACCATCTCCCGCGATAACTCCAAGAATACCCTCT |
| | | ATCTGCAAATGAACAGCCTGAGGGCCGAGGATACTGCAG |
| | | TGTACTACTGCGGGGGTTCAGGCTACGCGCTGCACGACGA |
| | | CTACTACGGATTGGACGTCTGGGGCCAAGGAACTCTTGTG |
| | | ACCGTGTCCTCTGGTGGAGGCGGATCAGGGGGTGGCGGAT |
| | | CTGGGGGTGGTGGTTCCGGGGGAGGAGGATCGCAGAGCG |
| | | CGCTGACTCAGCCTGCCTCCGTGAGCGGTTCGCCGGGACA |
| | | GTCCATTACCATTTCGTGCACCGGGACCTCCTCCGACGTG |
| | | GGAGGCTACAACTACGTGTCCTGGTACCAGCAGCATCCCG |
| | | GAAAGGCCCCGAAGCTGATGATCTACGAAGTGTCGAACA |
| | | GACTGCGGGGAGTCTCCAACCGCTTTTCCGGGTCCAAGTC |
| | | CGGCAACACCGCCAGCCTGACCATCAGCGGGCTCCAGGC |
| | | AGAAGATGAGGCTGACTATTACTGCTCCTCCTACACGTCA |
| | | AGCTCCGCCCTCTACGTGTTCGGGTCCGGGACCAAAGTCA |
| | | CTGTGCTGACCACTACCCCAGCACCGAGGCCACCCACCCC |
| | | GGCTCCTACCATCGCCTCCCAGCCTCTGTCCCTGCGTCCGG |
| | | AGGCATGTAGACCCGCAGCTGGTGGGGCCGTGCATACCCG |
| | | GGGTCTTGACTTCGCCTGCGATATCTACATTTGGGCCCCTC |
| | | TGGCTGGTACTTGCGGGGTCCTGCTGCTTTCACTCGTGATC |
| | | ACTCTTTACTGTAAGCGCGGTCGGAAGAAGCTGCTGTACA |
| | | TCTTTAAGCAACCCTTCATGAGGCCTGTGCAGACTACTCA |
| | | AGAGGAGGACGGCTGTTCATGCCGGTTCCCAGAGGAGGA |
| | | GGAAGGCGGCTGCGAACTGCGCGTGAAATTCAGCCGCAG |
| | | CGCAGATGCTCCAGCCTACCAGCAGGGGCAGAACCAGCT |
| | | CTACAACGAACTCAATCTTGGTCGGAGAGAGGAGTACGA |
| | | CGTGCTGGACAAGCGGAGAGGACGGGACCCAGAAATGGG |
| | | CGGGAAGCCGCGCAGAAAGAATCCCCAAGAGGGCCTGTA |
| | | CAACGAGCTCCAAAAGGATAAGATGGCAGAAGCCTATAG |
| | | CGAGATTGGTATGAAAGGGGAACGCAGAAGAGGCAAAGG |
| | | CCACGACGGACTGTACCAGGGACTCAGCACCGCCACCAA |
| | | GGACACCTATGACGCTCTTCACATGCAGGCCCTGCCGCCT |
| | | CGG |
| B61-10 | | |
| SEQ ID | HCDR1 | SYGMH |
| NO: 86 | (Kabat) | |
| SEQ ID | HCDR2 | VISYKGSNKYYADSVKG |
| NO: | (Kabat) | |
| 109 | | |
| SEQ ID | HCDR3 | SGYALHDDYYGLDV |
| NO: 88 | (Kabat) | |
| SEQ ID | HCDR1 | GFTFSSY |
| NO: 47 | (Chothia) | |
| SEQ ID | HCDR2 | SYKGSN |
| NO: | (Chothia) | |
| 110 | | |
| SEQ ID | HCDR3 | SGYALHDDYYGLDV |
| NO: 88 | (Chothia) | |
| SEQ ID | HCDR1 | GFTFSSYG |
| NO: 90 | (IMGT) | |
| SEQ ID | HCDR2 | ISYKGSNK |
| NO: | (IMGT) | |
| 111 | | |
| SEQ ID | HCDR3 | GGSGYALHDDYYGLDV |
| NO: 92 | (IMGT) | |
| SEQ ID | VH | QVQLVESGGGVVQPGRSLRLSCAASGFTFSSYGMHWVRQAP |
| NO: | | GKGLEWVAVISYKGSNKYYADSVKGRFTISRDNSKNTLYLQ |
| 112 | | MNSLRAEDTAVYYCGGSGYALHDDYYGLDVWGQGTLVTV |
| | | SS |
| SEQ ID | DNA VH | CAAGTGCAGCTTGTCGAATCGGGAGGCGGAGTGGTGCAG |
| NO: | | CCTGGACGATCGCTCCGGCTCTCATGTGCCGCGAGCGGAT |
| 113 | | TCACCTTCTCGAGCTACGGCATGCACTGGGTCAGACAAGC |
| | | CCCAGGAAAGGGCCTGGAATGGGTGGCTGTCATCTCGTAC |
| | | AAGGGCTCAAACAAGTACTACGCCGACTCCGTGAAGGGC |
| | | CGGTTCACCATCTCCCGCGATAACTCCAAGAATACCCTCT |
| | | ATCTGCAAATGAACAGCCTGAGGGCCGAGGATACTGCAG |
| | | TGTACTACTGCGGGGGTTCAGGCTACGCGCTGCACGACGA |
| | | CTACTACGGATTGGACGTCTGGGGCCAAGGAACTCTTGTG |
| | | ACCGTGTCCTCT |
| SEQ ID | LCDR1 | TGTSSDVGGYNYVS |
| NO: 95 | (Kabat) | |
| SEQ ID | LCDR2 | EVSNRLR |
| NO: | (Kabat) | |
| 114 | | |
| SEQ ID | LCDR3 | SSYTSSSTLYV |
| NO: 97 | (Kabat) | |
| SEQ ID | LCDR1 | TSSDVGGYNY |
| NO: 98 | (Chothia) | |
| SEQ ID | LCDR2 | EVS |
| NO: | (Chothia) | |
| 116 | | |
| SEQ ID | LCDR3 | YTSSSTLY |
| NO: | (Chothia) | |
| 100 | | |
| SEQ ID | LCDR1 | SSDVGGYNY |
| NO: | (IMGT) | |
| 101 | | |
| SEQ ID | LCDR2 | EVS |
| NO: | (IMGT) | |
| 116 | | |
| SEQ ID | LCDR3 | SSYTSSSTLYV |
| NO: 97 | (IMGT) | |
| SEQ ID | VL | QSALTQPASVSGSPGQSITISCTGTSSDVGGYNYVSWYQQHP |
| NO: | | GKAPKLMIYEVSNRLRGVSNRFSGSKSGNTASLTISGLQAED |
| 124 | | EADYYCSSYTSSSTLYVFGSGTKVTVL |
| SEQ ID | DNA VL | CAGAGCGCGCTGACTCAGCCTGCCTCCGTGAGCGGTTCGC |
| NO: | | CGGGACAGTCCATTACCATTTCGTGCACCGGGACCTCCTC |
| 125 | | CGACGTGGGAGGCTACAACTACGTGTCCTGGTACCAGCAG |
| | | CATCCCGGAAAGGCCCCGAAGCTGATGATCTACGAAGTGT |
| | | CGAACAGACTGCGGGGAGTCTCCAACCGCTTTTCCGGGTC |
| | | CAAGTCCGGCAACACCGCCAGCCTGACCATCAGCGGGCTC |
| | | CAGGCAGAAGATGAGGCTGACTATTACTGCTCCTCCTACA |
| | | CGTCAAGCTCCACCCTCTACGTGTTCGGGTCCGGGACCAA |
| | | AGTCACTGTGCTG |
| SEQ ID | Linker | GGGGSGGGGSGGGGSGGGGS |
| NO: 63 | | |
| SEQ ID | scFv (VH- | QVQLVESGGGVVQPGRSLRLSCAASGFTFSSYGMHWVRQAP |
| NO: | linker-VL) | GKGLEWVAVISYKGSNKYYADSVKGRFTISRDNSKNTLYLQ |
| 126 | | MNSLRAEDTAVYYCGGSGYALHDDYYGLDVWGQGTLVTV |
| | | SSGGGGSGGGGSGGGGSGGGGSQSALTQPASVSGSPGQSITI |
| | | SCTGTSSDVGGYNYVSWYQQHPGKAPKLMIYEVSNRLRGVS |
| | | NRFSGSKSGNTASLTISGLQAEDEADYYCSSYTSSSTLYVFGS |
| | | GTKVTVL |
| SEQ ID | DNA scFv | CAAGTGCAGCTTGTCGAATCGGGAGGCGGAGTGGTGCAG |
| NO: | | CCTGGACGATCGCTCCGGCTCTCATGTGCCGCGAGCGGAT |
| 127 | | TCACCTTCTCGAGCTACGGCATGCACTGGGTCAGACAAGC |
| | | CCCAGGAAAGGGCCTGGAATGGGTGGCTGTCATCTCGTAC |
| | | AAGGGCTCAAACAAGTACTACGCCGACTCCGTGAAGGGC |
| | | CGGTTCACCATCTCCCGCGATAACTCCAAGAATACCCTCT |
| | | ATCTGCAAATGAACAGCCTGAGGGCCGAGGATACTGCAG |
| | | TGTACTACTGCGGGGGTTCAGGCTACGCGCTGCACGACGA |
| | | CTACTACGGATTGGACGTCTGGGGCCAAGGAACTCTTGTG |
| | | ACCGTGTCCTCTGGTGGAGGCGGATCAGGGGGTGGCGGAT |
| | | CTGGGGGTGGTGGTTCCGGGGGAGGAGGATCGCAGAGCG |
| | | CGCTGACTCAGCCTGCCTCCGTGAGCGGTTCGCCGGGACA |
| | | GTCCATTACCATTTCGTGCACCGGGACCTCCTCCGACGTG |
| | | GGAGGCTACAACTACGTGTCCTGGTACCAGCAGCATCCCG |
| | | GAAAGGCCCCGAAGCTGATGATCTACGAAGTGTCGAACA |
| | | GACTGCGGGGAGTCTCCAACCGCTTTTCCGGGTCCAAGTC |
| | | CGGCAACACCGCCAGCCTGACCATCAGCGGGCTCCAGGC |
| | | AGAAGATGAGGCTGACTATTACTGCTCCTCCTACACGTCA |
| | | AGCTCCACCCTCTACGTGTTCGGGTCCGGGACCAAAGTCA |
| | | CTGTGCTG |
| SEQ ID | Full CAR | QVQLVESGGGVVQPGRSLRLSCAASGFTFSSYGMHWVRQAP |
| NO: | amino acid | GKGLEWVAVISYKGSNKYYADSVKGRFTISRDNSKNTLYLQ |
| 128 | sequence | MNSLRAEDTAVYYCGGSGYALHDDYYGLDVWGQGTLVTV |
| | | SSGGGGSGGGGSGGGGSGGGGSQSALTQPASVSGSPGQSITI |
| | | SCTGTSSDVGGYNYVSWYQQHPGKAPKLMIYEVSNRLRGVS |
| | | NRFSGSKSGNTASLTISGLQAEDEADYYCSSYTSSSTLYVFGS |
| | | GTKVTVLTTTPAPRPPTPAPTIASQPLSLRPEACRPAAGGAVH |
| | | TRGLDFACDIYIWAPLAGTCGVLLLSLVITLYCKRGRKKLLYI |
| | | FKQPFMRPVQTTQEEDGCSCRFPEEEEGGCELRVKFSRSADA |
| | | PAYQQGQNQLYNELNLGRREEYDVLDKRRGRDPEMGGKPR |
| | | RKNPQEGLYNELQKDKMAEAYSEIGMKGERRRGKGHDGLY |
| | | QGLSTATKDTYDALHMQALPPR |
| SEQ ID | Full CAR | CAAGTGCAGCTTGTCGAATCGGGAGGCGGAGTGGTGCAG |
| NO: | DNA | CCTGGACGATCGCTCCGGCTCTCATGTGCCGCGAGCGGAT |
| 129 | sequence | TCACCTTCTCGAGCTACGGCATGCACTGGGTCAGACAAGC |
| | | CCCAGGAAAGGGCCTGGAATGGGTGGCTGTCATCTCGTAC |
| | | AAGGGCTCAAACAAGTACTACGCCGACTCCGTGAAGGGC |
| | | CGGTTCACCATCTCCCGCGATAACTCCAAGAATACCCTCT |
| | | ATCTGCAAATGAACAGCCTGAGGGCCGAGGATACTGCAG |
| | | TGTACTACTGCGGGGGTTCAGGCTACGCGCTGCACGACGA |
| | | CTACTACGGATTGGACGTCTGGGGCCAAGGAACTCTTGTG |
| | | ACCGTGTCCTCTGGTGGAGGCGGATCAGGGGGTGGCGGAT |
| | | CTGGGGGTGGTGGTTCCGGGGGAGGAGGATCGCAGAGCG |
| | | CGCTGACTCAGCCTGCCTCCGTGAGCGGTTCGCCGGGACA |
| | | GTCCATTACCATTTCGTGCACCGGGACCTCCTCCGACGTG |
| | | GGAGGCTACAACTACGTGTCCTGGTACCAGCAGCATCCCG |
| | | GAAAGGCCCCGAAGCTGATGATCTACGAAGTGTCGAACA |
| | | GACTGCGGGGAGTCTCCAACCGCTTTTCCGGGTCCAAGTC |
| | | CGGCAACACCGCCAGCCTGACCATCAGCGGGCTCCAGGC |
| | | AGAAGATGAGGCTGACTATTACTGCTCCTCCTACACGTCA |
| | | AGCTCCACCCTCTACGTGTTCGGGTCCGGGACCAAAGTCA |
| | | CTGTGCTGACCACTACCCCAGCACCGAGGCCACCCACCCC |
| | | GGCTCCTACCATCGCCTCCCAGCCTCTGTCCCTGCGTCCGG |
| | | AGGCATGTAGACCCGCAGCTGGTGGGGCCGTGCATACCCG |
| | | GGGTCTTGACTTCGCCTGCGATATCTACATTTGGGCCCCTC |
| | | TGGCTGGTACTTGCGGGGTCCTGCTGCTTTCACTCGTGATC |
| | | ACTCTTTACTGTAAGCGCGGTCGGAAGAAGCTGCTGTACA |
| | | TCTTTAAGCAACCCTTCATGAGGCCTGTGCAGACTACTCA |
| | | AGAGGAGGACGGCTGTTCATGCCGGTTCCCAGAGGAGGA |
| | | GGAAGGCGGCTGCGAACTGCGCGTGAAATTCAGCCGCAG |
| | | CGCAGATGCTCCAGCCTACCAGCAGGGGCAGAACCAGCT |
| | | CTACAACGAACTCAATCTTGGTCGGAGAGAGGAGTACGA |
| | | CGTGCTGGACAAGCGGAGAGGACGGGACCCAGAAATGGG |
| | | CGGGAAGCCGCGCAGAAAGAATCCCCAAGAGGGCCTGTA |
| | | CAACGAGCTCCAAAAGGATAAGATGGCAGAAGCCTATAG |
| | | CGAGATTGGTATGAAAGGGGAACGCAGAAGAGGCAAAGG |
| | | CCACGACGGACTGTACCAGGGACTCAGCACCGCCACCAA |
| | | GGACACCTATGACGCTCTTCACATGCAGGCCCTGCCGCCT |
| | | CGG |

TABLE 7
Kabat CDRs of exemplary B cell-derived anti-BCMA molecules

| Kabat | HCDR1 | HCDR2 | HCDR3 | LCDR1 | LCDR2 | LCDR3 |
|---|---|---|---|---|---|---|
| PI61 | SYGMH | VISYDGSN | SGYALHD | TGTSSD | DVSNRPS | SSYTSSS |
| | (SEQ ID | KYYADSV | DYYGLDV | VGGYNY | (SEQ ID NO: | TLYV |
| | NO: 86) | KG (SEQ | (SEQ ID | VS (SEQ | 96) | (SEQ ID |
| | | ID NO: 87) | NO: 88) | ID NO: | | NO: 97) |
| | | | | 95) | | |
| B61-02 | SYGMH | VISYKGSN | SGYALHD | TGTSSD | EVSNRLR | SSYTSSS |
| | (SEQ ID | KYYADSV | DYYGLDV | VGGYNY | (SEQ ID NO: | ALYV |
| | NO: 86) | KG (SEQ | (SEQ ID | VS (SEQ | 114) | (SEQ ID |
| | | ID NO: | NO: 88) | ID NO: | | NO: 115) |
| | | 109) | | 95) | | |
| B61-10 | SYGMH | VISYKGSN | SGYALHD | TGTSSD | EVSNRLR | SSYTSSS |
| | (SEQ ID | KYYADSV | DYYGLDV | VGGYNY | (SEQ ID NO: | TLYV |
| | NO: 86) | KG (SEQ | (SEQ ID | VS (SEQ | 114) | (SEQ ID |
| | | ID NO: | NO: 88) | ID NO: | | NO: 97) |
| | | 109) | | 95) | | |
| Consensus | SYGMH | VISYXGSN | SGYALHD | TGTSSD | X₁VSNRX₂X₃, | SSYTSSS |
| | (SEQ ID | KYYADSV | DYYGLDV | VGGYNY | wherein X₁ | XLYV, |
| | NO: 86) | KG, | (SEQ ID | VS (SEQ | is D or E; X₂ | wherein |
| | | wherein X | NO: 88) | ID NO: | is P or L; and | X is T or |
| | | is D or K | | 95) | X₃ is S or R | A (SEQ |
| | | (SEQ ID | | | (SEQ ID NO: | ID NO: |
| | | NO: 130) | | | 131) | 132) |

TABLE 8
Chothia CDRs of exemplary B cell-derived anti-BCMA molecules

| Chothia | HCDR1 | HCDR2 | HCDR3 | LCDR1 | LCDR2 | LCDR3 |
|---|---|---|---|---|---|---|
| PI61 | GFTFSS | SYDGSN | SGYALHDDY | TSSDVGG | DVS | YTSSSTL |
| | Y (SEQ | (SEQ ID | YGLDV (SEQ | YNY (SEQ | (SEQ ID | Y (SEQ ID |
| | ID NO: | NO: 89) | ID NO: 88) | ID NO: 98) | NO: 99) | NO: 100) |
| | 47) | | | | | |
| B61-02 | GFTFSS | SYKGSN | SGYALHDDY | TSSDVGG | EVS | YTSSSAL |
| | Y (SEQ | (SEQ ID | YGLDV (SEQ | YNY (SEQ | (SEQ ID | Y (SEQ ID |
| | ID NO: | NO: 110) | ID NO: 88) | ID NO: 98) | NO: 116) | NO: 117) |
| | 47) | | | | | |
| B61-10 | GFTFSS | SYKGSN | SGYALHDDY | TSSDVGG | EVS | YTSSSTL |
| | Y (SEQ | (SEQ ID | YGLDV (SEQ | YNY (SEQ | (SEQ ID | Y (SEQ ID |
| | ID NO: | NO: 110) | ID NO: 88) | ID NO: 98) | NO: 116) | NO: 100) |
| | 47) | | | | | |
| Consensus | GFTFSS | SYXGSN, | SGYALHDDY | TSSDVGG | XVS, | YTSSSXL |
| | Y (SEQ | wherein X | YGLDV (SEQ | YNY (SEQ | wherein X | Y, wherein |
| | ID NO: | is D or K | ID NO: 88) | ID NO: 98) | is D or E | X is T or A |
| | 47) | (SEQ ID | | | (SEQ ID | (SEQ ID |
| | | NO: 133) | | | NO: 134) | NO: 135) |

TABLE 9
IMGT CDRs of exemplary B cell-derived anti-BCMA molecules

| IMGT | HCDR1 | HCDR2 | HCDR3 | LCDR1 | LCDR2 | LCDR3 |
|---|---|---|---|---|---|---|
| PI61 | GFTFSSY | ISYDGSN | GGSGYALHD | SSDVGGY | DVS | SSYTSSST |
| | G (SEQ ID | K (SEQ | DYYGLDV | NY (SEQ | (SEQ ID | LYV (SEQ |
| | NO: 90) | ID NO: | (SEQ ID NO: | ID NO: | NO: 99) | ID NO: 97) |
| | | 91) | 92) | 101) | | |
| B61-02 | GFTFSSY | ISYKGSN | GGSGYALHD | SSDVGGY | EVS | SSYTSSSA |
| | G (SEQ ID | K (SEQ | DYYGLDV | NY (SEQ | (SEQ ID | LYV (SEQ |
| | NO: 90) | ID NO: | (SEQ ID NO: | ID NO: | NO: | ID NO: |
| | | 111) | 92) | 101) | 116) | 115) |
| B61-10 | GFTFSSY | ISYKGSN | GGSGYALHD | SSDVGGY | EVS | SSYTSSST |
| | G (SEQ ID | K (SEQ | DYYGLDV | NY (SEQ | (SEQ ID | LYV (SEQ |
| | NO: 90) | ID NO: | (SEQ ID NO: | ID NO: | NO: | ID NO: 97) |
| | | 111) | 92) | 101) | 116) | |
| Consensus | GFTFSSY | ISYXGSN | GGSGYALHD | SSDVGGY | XVS, | SSYTSSSX |
| | G (SEQ ID | K, | DYYGLDV | NY (SEQ | wherein | LYV, |
| | NO: 90) | wherein X | (SEQ ID NO: | ID NO: | X is D | wherein X |
| | | is D or K | 92) | 101) | or E | is T or A |
| | | (SEQ ID | | | (SEQ ID | (SEQ ID |
| | | NO: 136) | | | NO: | NO: 132) |
| | | | | | 134) | |

TABLE 14
Amino acid and nucleic acid sequences of
exemplary anti-BCMA molecules based on PI61

| Identification | Protein sequence | DNA sequence (5′-3′) |
|---|---|---|
| Signal peptide | MALPVTALLLPLALLLHA | Atggccctccctgtcaccgctctgttgctgccgcttgctct |
| | ARP (SEQ ID NO: 2) | gctgctccacgcagcgcgaccg (SEQ ID NO: |
| | | 252) |
| ScFv PI61 | QVQLQESGGGVVQPGRS | CaggtacaattgcaggagtctggaggcggtgtgGtgca |
| | LRLSCAASGFTFSSYGMH | acccggtcgcagcttgcgcctgagttgtGctgcgtctgg |
| | WVRQAPGKGLEWVAVIS | atttacattttcatcttacggaAtgcattgggtacgccagg |
| | YDGSNKYYADSVKGRFT | caccggggaaaggcCttgaatgggtggctgtaatttcat |
| | ISRDNSKNTLYLQMNSLR | acgatggtTccaacaaatactatgctgactcagtcaagg |
| | AEDTAVYYCGGSGYALH | gtCgatttacaattagtcgggacaactccaagaacAccc |
| | DDYYGLDVWGQGTLVT | tttatcttcaaatgaattcccttagagcaGaggatacggcg |
| | VSSGGGGSGGGGSGGGG | gtctattactgtggtggcagtGgttatgcacttcatgatgat |
| | SQSALTQPASVSGSPGQSI | tactatggcttgGatgtctgggggcaagggacgcttgtaa |
| | TISCTGTSSDVGGYNYVS | ctgtaTcctctggtggtggtggtagtggtgggggaggc |
| | WYQQHPGKAPKLMIYDV | TccggcggtggcggctctcaatctgctctgactCaacca |
| | SNRPSGVSNRFSGSKSGN | gcaagcgtatcagggtcaccgggacagAgtattaccat |
| | TASLTISGLQAEDEADYY | aagttgcacggggacctctagcGatgtaggggggtata |
| | CSSYTSSSTLYVFGSGTK | attatgtatcttggtatCaacaacaccccgggaaagcccc |
| | VTVL (SEQ ID NO: 105) | taaattgatgAtctacgacgtgagcaatcgacctagtggc |
| | | gtaTcaaatcgcttctctggtagcaagagtgggaatAcg |
| | | gcgtcccttactattagcggattgcaagcaGaagatgag |
| | | gccgattactactgcagctcctatActagctcttctacattg |
| | | tacgtctttgggagcggaacaaaagtaacagtactc |
| | | (SEQ ID NO: 253) |
| Transmembrane | TTTPAPRPPTPAPTIASQP | AcaacaacacctgccccgagaccgcctacaccaGccc |
| domain and | LSLRPEACRPAAGGAVHT | cgactattgccagccagcctctgagcctcAggcctgag |
| hinge | RGLDFACDIYIWAPLAGT | gcctgtaggcccgcagcgggcggcGcagttcatacac |
| | CGVLLLSLVITLYC (SEQ | ggggcttggatttcgcttgtGatatttatatttgggctccttt |
| | ID NO: 202) | ggcggggacaTgtggcgtgctgcttctgtcacttgttatta |
| | | cactgtactgt (SEQ ID NO: 254) |
| 4-1BB | KRGRKKLLYIFKQPFMRP | AaacgcgggcgaaaaaaattgctgtatatttttAagcag |
| | VQTTQEEDGCSCRFPEEE | ccatttatgaggcccgttcagacgacgCaggaggagga |
| | EGGCEL (SEQ ID NO: 14) | cggttgctcttgcaggttcccagaagaggaagaagggg |
| | | gctgtgaattg (SEQ ID NO: 255) |
| CD3zeta | RVKFSRSADAPAYQQGQ | CgggttaaattttcaagatccgcagacgctccaGcatac |
| | NQLYNELNLGRREEYDV | caacagggacaaaaccaactctataacGagctgaatctt |
| | LDKRRGRDPEMGGKPRR | ggaagaagggaggaatatgatGtgctggataaacggcg |
| | KNPQEGLYNELQKDKMA | cggtagagatccggagAtgggcggaaaaccaaggcg |
| | EAYSEIGMKGERRRGKG | aaaaaaccctcagGagggactctacaacgaactgcaga |
| | HDGLYQGLSTATKDTYD | aagacaaaAtggcggaggcttattccgaaataggcatg |
| | ALHMQALPPR (SEQ ID | aagGgcgagcggaggcgagggaaagggcacgacgg |
| | NO: 20) | aCtgtatcaaggcctctcaaccgcgactaaggatAcgta |
| | | cgacgccctgcacatgcaggccctgcctccgaga |
| | | (SEQ ID NO: 256) |
| PI61 full CAR | MALPVTALLLPLALLLHA | ATGGCCCTCCCTGTCACCGCTCTGT |
| construct | ARPQVQLQESGGGVVQP | TGCTGCCGCTTGCTCTGCTGCTCCA |
| | GRSLRLSCAASGFTFSSY | CGCAGCGCGACCGCAGGTACAATT |
| | GMHWVRQAPGKGLEWV | GCAGGAGTCTGGAGGCGGTGTGGT |
| | AVISYDGSNKYYADSVK | GCAACCCGGTCGCAGCTTGCGCCT |
| | GRFTISRDNSKNTLYLQM | GAGTTGTGCTGCGTCTGGATTTACA |
| | NSLRAEDTAVYYCGGSG | TTTTCATCTTACGGAATGCATTGGG |
| | YALHDDYYGLDVWGQG | TACGCCAGGCACCGGGGAAAGGCC |
| | TLVTVSSGGGGSGGGGS | TTGAATGGGTGGCTGTAATTTCATA |
| | GGGGSQSALTQPASVSGS | CGATGGTTCCAACAAATACTATGCT |
| | PGQSITISCTGTSSDVGGY | GACTCAGTCAAGGGTCGATTTACA |
| | NYVSWYQQHPGKAPKL | ATTAGTCGGGACAACTCCAAGAAC |
| | MIYDVSNRPSGVSNRFSG | ACCCTTTATCTTCAAATGAATTCCC |
| | SKSGNTASLTISGLQAED | TTAGAGCAGAGGATACGGCGGTCT |
| | EADYYCSSYTSSSTLYVF | ATTACTGTGGTGGCAGTGGTTATGC |
| | GSGTKVTVLTTTPAPRPP | ACTTCATGATGATTACTATGGCTTG |
| | TPAPTIASQPLSLRPEACR | GATGTCTGGGGGCAAGGGACGCTT |
| | PAAGGAVHTRGLDFACD | GTAACTGTATCCTCTGGTGGTGGTG |
| | IYIWAPLAGTCGVLLLSL | GTAGTGGTGGGGGAGGCTCCGGCG |
| | VITLYCKRGRKKLLYIFK | GTGGCGGCTCTCAATCTGCTCTGAC |
| | QPFMRPVQTTQEEDGCSC | TCAACCAGCAAGCGTATCAGGGTC |
| | RFPEEEEGGCELRVKFSR | ACCGGGACAGAGTATTACCATAAG |
| | SADAPAYQQGQNQLYNE | TTGCACGGGGACCTCTAGCGATGT |
| | LNLGRREEYDVLDKRRG | AGGGGGGTATAATTATGTATCTTGG |
| | RDPEMGGKPRRKNPQEG | TATCAACAACACCCCGGGAAAGCC |
| | LYNELQKDKMAEAYSEI | CCTAAATTGATGATCTACGACGTGA |
| | GMKGERRRGKGHDGLY | GCAATCGACCTAGTGGCGTATCAA |
| | QGLSTATKDTYDALHMQ | ATCGCTTCTCTGGTAGCAAGAGTGG |
| | ALPPR (SEQ ID NO: 257) | GAATACGGCGTCCCTTACTATTAGC |
| | | GGATTGCAAGCAGAAGATGAGGCC |
| | | GATTACTACTGCAGCTCCTATACTA |
| | | GCTCTTCTACATTGTACGTCTTTGG |
| | | GAGCGGAACAAAAGTAACAGTACT |
| | | CACAACAACACCTGCCCCGAGACC |
| | | GCCTACACCAGCCCCGACTATTGCC |
| | | AGCCAGCCTCTGAGCCTCAGGCCT |
| | | GAGGCCTGTAGGCCCGCAGCGGGC |
| | | GGCGCAGTTCATACACGGGGCTTG |
| | | GATTTCGCTTGTGATATTTATATTT |
| | | GGGCTCCTTTGGCGGGGACATGTG |
| | | GCGTGCTGCTTCTGTCACTTGTTAT |
| | | TACACTGTACTGTAAACGCGGGCG |
| | | AAAAAAATTGCTGTATATTTTTAAG |
| | | CAGCCATTTATGAGGCCCGTTCAGA |
| | | CGACGCAGGAGGAGGACGGTTGCT |
| | | CTTGCAGGTTCCCAGAAGAGGAAG |
| | | AAGGGGGCTGTGAATTGCGGGTTA |
| | | AATTTTCAAGATCCGCAGACGCTCC |
| | | AGCATACCAACAGGGACAAAACCA |
| | | ACTCTATAACGAGCTGAATCTTGGA |
| | | AGAAGGGAGGAATATGATGTGCTG |
| | | GATAAACGGCGCGGTAGAGATCCG |
| | | GAGATGGGCGGAAAACCAAGGCGA |
| | | AAAAACCCTCAGGAGGGACTCTAC |
| | | AACGAACTGCAGAAAGACAAAATG |
| | | GCGGAGGCTTATTCCGAAATAGGC |
| | | ATGAAGGGCGAGCGGAGGCGAGG |
| | | GAAAGGGCACGACGGACTGTATCA |
| | | AGGCCTCTCAACCGCGACTAAGGA |
| | | TACGTACGACGCCCTGCACATGCA |
| | | GGCCCTGCCTCCGAGA (SEQ ID NO: |
| | | 258) |
| PI61 mature | QVQLQESGGGVVQPGRS | |
| CAR protein | LRLSCAASGFTFSSYGMH | |
| | WVRQAPGKGLEWVAVIS | |
| | YDGSNKYYADSVKGRFT | |
| | ISRDNSKNTLYLQMNSLR | |
| | AEDTAVYYCGGSGYALH | |
| | DDYYGLDVWGQGTLVT | |
| | VSSGGGGSGGGGSGGGG | |
| | SQSALTQPASVSGSPGQSI | |
| | TISCTGTSSDVGGYNYVS | |
| | WYQQHPGKAPKLMIYDV | |
| | SNRPSGVSNRFSGSKSGN | |
| | TASLTISGLQAEDEADYY | |
| | CSSYTSSSTLYVFGSGTK | |
| | VTVLTTTPAPRPPTPAPTI | |
| | ASQPLSLRPEACRPAAGG | |
| | AVHTRGLDFACDIYIWAP | |
| | LAGTCGVLLLSLVITLYC | |
| | KRGRKKLLYIFKQPFMRP | |
| | VQTTQEEDGCSCRFPEEE | |
| | EGGCELRVKFSRSADAPA | |
| | YQQGQNQLYNELNLGRR | |
| | EEYDVLDKRRGRDPEMG | |
| | GKPRRKNPQEGLYNELQ | |
| | KDKMAEAYSEIGMKGER | |
| | RRGKGHDGLYQGLSTAT | |
| | KDTYDALHMQALPPR | |
| | (SEQ ID NO: 107) | |

TABLE 10
Amino acid and nucleic acid sequences of
exemplary hybridoma-derived anti-BCMA molecules

| SEQ | Name/ | |
|---|---|---|
| ID NO | Description | Sequence |
| Hy03 | | |
| SEQ ID | HCDR1 | GFWMS |
| NO: | (Kabat) | |
| 137 | | |
| SEQ ID | HCDR2 | NIKQDGSEKYYVDSVRG |
| NO: | (Kabat) | |
| 138 | | |
| SEQ ID | HCDR3 | ALDYYGMDV |
| NO: | (Kabat) | |
| 139 | | |
| SEQ ID | HCDR1 | GFTFSGF |
| NO: | (Chothia) | |
| 140 | | |
| SEQ ID | HCDR2 | KQDGSE |
| NO: | (Chothia) | |
| 141 | | |
| SEQ ID | HCDR3 | ALDYYGMDV |
| NO: | (Chothia) | |
| 139 | | |
| SEQ ID | HCDR1 | GFTFSGFW |
| NO: | (IMGT) | |
| 142 | | |
| SEQ ID | HCDR2 | IKQDGSEK |
| NO: | (IMGT) | |
| 143 | | |
| SEQ ID | HCDR3 | ARALDYYGMDV |
| NO: | (IMGT) | |
| 144 | | |
| SEQ ID | VH | EVQLVESGGGLVQPGGSLRLSCAASGFTFSGFWMSWVRQA |
| NO: | | PGKGLEWVANIKQDGSEKYYVDSVRGRFTISRDNAKNSLYL |
| 145 | | QMNSLRAEDTAVYYCARALDYYGMDVWGQGTTVTVSS |
| SEQ ID | DNA VH | GAAGTGCAACTGGTGGAGAGCGGTGGAGGGCTTGTCCAG |
| NO: | | CCCGGAGGATCGCTGCGGCTGTCCTGTGCTGCGTCCGGGT |
| 146 | | TCACCTTCTCCGGCTTCTGGATGTCCTGGGTCAGACAGGC |
| | | ACCGGGAAAGGGCCTCGAATGGGTGGCCAACATCAAGCA |
| | | GGATGGCTCCGAGAAGTACTACGTCGACTCCGTGAGAGG |
| | | CCGCTTCACCATCTCCCGGGACAACGCCAAGAACTCGCT |
| | | GTACCTCCAAATGAATAGCCTCAGGGCGGAAGATACTGC |
| | | TGTGTATTACTGCGCACGCGCCCTTGACTACTACGGCATG |
| | | GACGTCTGGGGCCAAGGGACCACTGTGACCGTGTCTAGC |
| SEQ ID | LCDR1 | RSSQSLLDSDDGNTYLD |
| NO: | (Kabat) | |
| 147 | | |
| SEQ ID | LCDR2 | TLSYRAS |
| NO: | (Kabat) | |
| 148 | | |
| SEQ ID | LCDR3 | TQRLEFPSIT |
| NO: | (Kabat) | |
| 149 | | |
| SEQ ID | LCDR1 | SQSLLDSDDGNTY |
| NO: | (Chothia) | |
| 150 | | |
| SEQ ID | LCDR2 | TLS |
| NO: | (Chothia) | |
| 151 | | |
| SEQ ID | LCDR3 | RLEFPSI |
| NO: | (Chothia) | |
| 152 | | |
| SEQ ID | LCDR1 | QSLLDSDDGNTY |
| NO: | (IMGT) | |
| 153 | | |
| SEQ ID | LCDR2 | TLS |
| NO: | (IMGT) | |
| 151 | | |
| SEQ ID | LCDR3 | TQRLEFPSIT |
| NO: | (IMGT) | |
| 149 | | |
| SEQ ID | VL | DIVMTQTPLSLPVTPGEPASISCRSSQSLLDSDDGNTYLDWY |
| NO: | | LQKPGQSPRLLIYTLSYRASGVPDRFSGSGSGTDFTLKISRVE |
| 154 | | AEDVGLYYCTQRLEFPSITFGQGTRLEIK |
| SEQ ID | DNA VL | GATATCGTGATGACCCAGACTCCCCTGTCCCTGCCTGTGA |
| NO: | | CTCCCGGAGAACCAGCCTCCATTTCCTGCCGGTCCTCCCA |
| 155 | | GTCCCTGCTGGACAGCGACGACGGCAACACTTACCTGGA |
| | | CTGGTACTTGCAGAAGCCGGGCCAATCGCCTCGCCTGCTG |
| | | ATCTATACCCTGTCATACCGGGCCTCAGGAGTGCCTGACC |
| | | GCTTCTCGGGATCAGGGAGCGGGACCGATTTCACCCTGA |
| | | AAATTTCCCGAGTGGAAGCCGAGGACGTCGGACTGTACT |
| | | ACTGCACCCAGCGCCTCGAATTCCCGTCGATTACGTTTGG |
| | | ACAGGGTACCCGGCTTGAGATCAAG |
| SEQ ID | Linker | GGGGSGGGGSGGGGSGGGGS |
| NO: 63 | | |
| SEQ ID | scFv (VH- | EVQLVESGGGLVQPGGSLRLSCAASGFTFSGFWMSWVRQA |
| NO: | linker-VL) | PGKGLEWVANIKQDGSEKYYVDSVRGRFTISRDNAKNSLYL |
| 156 | | QMNSLRAEDTAVYYCARALDYYGMDVWGQGTTVTVSSG |
| | | GGGSGGGGSGGGGSGGGGSDIVMTQTPLSLPVTPGEPASISC |
| | | RSSQSLLDSDDGNTYLDWYLQKPGQSPRLLIYTLSYRASGV |
| | | PDRFSGSGSGTDFTLKISRVEAEDVGLYYCTQRLEFPSITFGQ |
| | | GTRLEIK |
| SEQ ID | DNA scFv | GAAGTGCAACTGGTGGAGAGCGGTGGAGGGCTTGTCCAG |
| NO: | | CCCGGAGGATCGCTGCGGCTGTCCTGTGCTGCGTCCGGGT |
| 157 | | TCACCTTCTCCGGCTTCTGGATGTCCTGGGTCAGACAGGC |
| | | ACCGGGAAAGGGCCTCGAATGGGTGGCCAACATCAAGCA |
| | | GGATGGCTCCGAGAAGTACTACGTCGACTCCGTGAGAGG |
| | | CCGCTTCACCATCTCCCGGGACAACGCCAAGAACTCGCT |
| | | GTACCTCCAAATGAATAGCCTCAGGGCGGAAGATACTGC |
| | | TGTGTATTACTGCGCACGCGCCCTTGACTACTACGGCATG |
| | | GACGTCTGGGGCCAAGGGACCACTGTGACCGTGTCTAGC |
| | | GGAGGCGGAGGTTCAGGGGGCGGTGGATCAGGCGGAGG |
| | | AGGATCGGGGGGTGGTGGATCGGATATCGTGATGACCCA |
| | | GACTCCCCTGTCCCTGCCTGTGACTCCCGGAGAACCAGCC |
| | | TCCATTTCCTGCCGGTCCTCCCAGTCCCTGCTGGACAGCG |
| | | ACGACGGCAACACTTACCTGGACTGGTACTTGCAGAAGC |
| | | CGGGCCAATCGCCTCGCCTGCTGATCTATACCCTGTCATA |
| | | CCGGGCCTCAGGAGTGCCTGACCGCTTCTCGGGATCAGG |
| | | GAGCGGGACCGATTTCACCCTGAAAATTTCCCGAGTGGA |
| | | AGCCGAGGACGTCGGACTGTACTACTGCACCCAGCGCCT |
| | | CGAATTCCCGTCGATTACGTTTGGACAGGGTACCCGGCTT |
| | | GAGATCAAG |
| SEQ ID | Full CAR | EVQLVESGGGLVQPGGSLRLSCAASGFTFSGFWMSWVRQA |
| NO: | amino acid | PGKGLEWVANIKQDGSEKYYVDSVRGRFTISRDNAKNSLYL |
| 158 | sequence | QMNSLRAEDTAVYYCARALDYYGMDVWGQGTTVTVSSG |
| | | GGGSGGGGSGGGGSGGGGSDIVMTQTPLSLPVTPGEPASISC |
| | | RSSQSLLDSDDGNTYLDWYLQKPGQSPRLLIYTLSYRASGV |
| | | PDRFSGSGSGTDFTLKISRVEAEDVGLYYCTQRLEFPSITFGQ |
| | | GTRLEIKTTTPAPRPPTPAPTIASQPLSLRPEACRPAAGGAVH |
| | | TRGLDFACDIYIWAPLAGTCGVLLLSLVITLYCKRGRKKLLY |
| | | IFKQPFMRPVQTTQEEDGCSCRFPEEEEGGCELRVKFSRSAD |
| | | APAYQQGQNQLYNELNLGRREEYDVLDKRRGRDPEMGGK |
| | | PRRKNPQEGLYNELQKDKMAEAYSEIGMKGERRRGKGHD |
| | | GLYQGLSTATKDTYDALHMQALPPR |
| SEQ ID | Full CAR | GAAGTGCAACTGGTGGAGAGCGGTGGAGGGCTTGTCCAG |
| NO: | DNA | CCCGGAGGATCGCTGCGGCTGTCCTGTGCTGCGTCCGGGT |
| 159 | sequence | TCACCTTCTCCGGCTTCTGGATGTCCTGGGTCAGACAGGC |
| | | ACCGGGAAAGGGCCTCGAATGGGTGGCCAACATCAAGCA |
| | | GGATGGCTCCGAGAAGTACTACGTCGACTCCGTGAGAGG |
| | | CCGCTTCACCATCTCCCGGGACAACGCCAAGAACTCGCT |
| | | GTACCTCCAAATGAATAGCCTCAGGGCGGAAGATACTGC |
| | | TGTGTATTACTGCGCACGCGCCCTTGACTACTACGGCATG |
| | | GACGTCTGGGGCCAAGGGACCACTGTGACCGTGTCTAGC |
| | | GGAGGCGGAGGTTCAGGGGGCGGTGGATCAGGCGGAGG |
| | | AGGATCGGGGGGTGGTGGATCGGATATCGTGATGACCCA |
| | | GACTCCCCTGTCCCTGCCTGTGACTCCCGGAGAACCAGCC |
| | | TCCATTTCCTGCCGGTCCTCCCAGTCCCTGCTGGACAGCG |
| | | ACGACGGCAACACTTACCTGGACTGGTACTTGCAGAAGC |
| | | CGGGCCAATCGCCTCGCCTGCTGATCTATACCCTGTCATA |
| | | CCGGGCCTCAGGAGTGCCTGACCGCTTCTCGGGATCAGG |
| | | GAGCGGGACCGATTTCACCCTGAAAATTTCCCGAGTGGA |
| | | AGCCGAGGACGTCGGACTGTACTACTGCACCCAGCGCCT |
| | | CGAATTCCCGTCGATTACGTTTGGACAGGGTACCCGGCTT |
| | | GAGATCAAGACCACTACCCCAGCACCGAGGCCACCCACC |
| | | CCGGCTCCTACCATCGCCTCCCAGCCTCTGTCCCTGCGTC |
| | | CGGAGGCATGTAGACCCGCAGCTGGTGGGGCCGTGCATA |
| | | CCCGGGGTCTTGACTTCGCCTGCGATATCTACATTTGGGC |
| | | CCCTCTGGCTGGTACTTGCGGGGTCCTGCTGCTTTCACTC |
| | | GTGATCACTCTTTACTGTAAGCGCGGTCGGAAGAAGCTG |
| | | CTGTACATCTTTAAGCAACCCTTCATGAGGCCTGTGCAGA |
| | | CTACTCAAGAGGAGGACGGCTGTTCATGCCGGTTCCCAG |
| | | AGGAGGAGGAAGGCGGCTGCGAACTGCGCGTGAAATTCA |
| | | GCCGCAGCGCAGATGCTCCAGCCTACCAGCAGGGGCAGA |
| | | ACCAGCTCTACAACGAACTCAATCTTGGTCGGAGAGAGG |
| | | AGTACGACGTGCTGGACAAGCGGAGAGGACGGGACCCA |
| | | GAAATGGGCGGGAAGCCGCGCAGAAAGAATCCCCAAGA |
| | | GGGCCTGTACAACGAGCTCCAAAAGGATAAGATGGCAGA |
| | | AGCCTATAGCGAGATTGGTATGAAAGGGGAACGCAGAAG |
| | | AGGCAAAGGCCACGACGGACTGTACCAGGGACTCAGCAC |
| | | CGCCACCAAGGACACCTATGACGCTCTTCACATGCAGGC |
| | | CCTGCCGCCTCGG |
| Hy52 | | |
| SEQ ID | HCDR1 | SFRMN |
| NO: | (Kabat) | |
| 160 | | |
| SEQ ID | HCDR2 | SISSSSSYIYYADSVKG |
| NO: | (Kabat) | |
| 161 | | |
| SEQ ID | HCDR3 | WLSYYGMDV |
| NO: | (Kabat) | |
| 162 | | |
| SEQ ID | HCDR1 | GFTFSSF |
| NO: | (Chothia) | |
| 163 | | |
| SEQ ID | HCDR2 | SSSSSY |
| NO: | (Chothia) | |
| 164 | | |
| SEQ ID | HCDR3 | WLSYYGMDV |
| NO: | (Chothia) | |
| 162 | | |
| SEQ ID | HCDR1 | GFTFSSFR |
| NO: | (IMGT) | |
| 165 | | |
| SEQ ID | HCDR2 | ISSSSSYI |
| NO: | (IMGT) | |
| 166 | | |
| SEQ ID | HCDR3 | ARWLSYYGMDV |
| NO: | (IMGT) | |
| 167 | | |
| SEQ ID | VH | EVQLVESGGGLVKPGGSLRLSCAASGFTFSSFRMNWVRQAP |
| NO: | | GKGLEWVSSISSSSSYIYYADSVKGRFTISRDNAKNSLYLQM |
| 168 | | NSLRAEDTAVYYCARWLSYYGMDVWGQGTTVTVSS |
| SEQ ID | DNA VH | GAAGTGCAACTGGTGGAGAGCGGTGGAGGGCTTGTCAAG |
| NO: | | CCCGGAGGATCGCTGCGGCTGTCCTGTGCTGCGTCCGGGT |
| 169 | | TCACCTTCTCCTCGTTCCGCATGAACTGGGTCAGACAGGC |
| | | ACCGGGAAAGGGCCTCGAATGGGTGTCCTCAATCTCATC |
| | | GTCCTCGTCCTACATCTACTACGCCGACTCCGTGAAAGGC |
| | | CGCTTCACCATCTCCCGGGACAACGCCAAGAACTCGCTGT |
| | | ACCTCCAAATGAATAGCCTCAGGGCGGAAGATACTGCTG |
| | | TGTATTACTGCGCACGCTGGCTTTCCTACTACGGCATGGA |
| | | CGTCTGGGGCCAAGGGACCACTGTGACCGTGTCTAGC |
| SEQ ID | LCDR1 | RSSQSLLDSDDGNTYLD |
| NO: | (Kabat) | |
| 147 | | |
| SEQ ID | LCDR2 | TLSFRAS |
| NO: | (Kabat) | |
| 170 | | |
| SEQ ID | LCDR3 | MQRIGFPIT |
| NO: | (Kabat) | |
| 171 | | |
| SEQ ID | LCDR1 | SQSLLDSDDGNTY |
| NO: | (Chothia) | |
| 150 | | |
| SEQ ID | LCDR2 | TLS |
| NO: | (Chothia) | |
| 151 | | |
| SEQ ID | LCDR3 | RIGFPI |
| NO: | (Chothia) | |
| 172 | | |
| SEQ ID | LCDR1 | QSLLDSDDGNTY |
| NO: | (IMGT) | |
| 153 | | |
| SEQ ID | LCDR2 | TLS |
| NO: | (IMGT) | |
| 151 | | |
| SEQ ID | LCDR3 | MQRIGFPIT |
| NO: | (IMGT) | |
| 171 | | |
| SEQ ID | VL | DIVMTQTPLSLPVTPGEPASISCRSSQSLLDSDDGNTYLDWY |
| NO: | | LQKPGQSPQLLIYTLSFRASGVPDRFSGSGSGTDFTLKIRRVE |
| 173 | | AEDVGVYYCMQRIGFPITFGQGTRLEIK |
| SEQ ID | DNA VL | GATATCGTGATGACCCAGACTCCCCTGTCCCTGCCTGTGA |
| NO: | | CTCCCGGAGAACCAGCCTCCATTTCCTGCCGGTCCTCCCA |
| 174 | | GTCCCTGCTGGACAGCGACGACGGCAACACTTACCTGGA |
| | | CTGGTACTTGCAGAAGCCGGGCCAATCGCCTCAGCTGCT |
| | | GATCTATACCCTGTCATTCCGGGCCTCAGGAGTGCCTGAC |
| | | CGCTTCTCGGGATCAGGGAGCGGGACCGATTTCACCCTG |
| | | AAAATTAGGCGAGTGGAAGCCGAGGACGTCGGAGTGTAC |
| | | TACTGCATGCAGCGCATCGGCTTCCCGATTACGTTTGGAC |
| | | AGGGTACCCGGCTTGAGATCAAG |
| SEQ ID | Linker | GGGGSGGGGSGGGGSGGGGS |
| NO: 63 | | |
| SEQ ID | scFv (VH- | EVQLVESGGGLVKPGGSLRLSCAASGFTFSSFRMNWVRQAP |
| NO: | linker-VL) | GKGLEWVSSISSSSSYIYYADSVKGRFTISRDNAKNSLYLQM |
| 175 | | NSLRAEDTAVYYCARWLSYYGMDVWGQGTTVTVSSGGGG |
| | | SGGGGSGGGGSGGGGSDIVMTQTPLSLPVTPGEPASISCRSS |
| | | QSLLDSDDGNTYLDWYLQKPGQSPQLLIYTLSFRASGVPDR |
| | | FSGSGSGTDFTLKIRRVEAEDVGVYYCMQRIGFPITFGQGTR |
| | | LEIK |
| SEQ ID | DNA scFv | GAAGTGCAACTGGTGGAGAGCGGTGGAGGGCTTGTCAAG |
| NO: | | CCCGGAGGATCGCTGCGGCTGTCCTGTGCTGCGTCCGGGT |
| 176 | | TCACCTTCTCCTCGTTCCGCATGAACTGGGTCAGACAGGC |
| | | ACCGGGAAAGGGCCTCGAATGGGTGTCCTCAATCTCATC |
| | | GTCCTCGTCCTACATCTACTACGCCGACTCCGTGAAAGGC |
| | | CGCTTCACCATCTCCCGGGACAACGCCAAGAACTCGCTGT |
| | | ACCTCCAAATGAATAGCCTCAGGGCGGAAGATACTGCTG |
| | | TGTATTACTGCGCACGCTGGCTTTCCTACTACGGCATGGA |
| | | CGTCTGGGGCCAAGGGACCACTGTGACCGTGTCTAGCGG |
| | | AGGCGGAGGTTCAGGGGGCGGTGGATCAGGCGGAGGAG |
| | | GATCGGGGGGTGGTGGATCGGATATCGTGATGACCCAGA |
| | | CTCCCCTGTCCCTGCCTGTGACTCCCGGAGAACCAGCCTC |
| | | CATTTCCTGCCGGTCCTCCCAGTCCCTGCTGGACAGCGAC |
| | | GACGGCAACACTTACCTGGACTGGTACTTGCAGAAGCCG |
| | | GGCCAATCGCCTCAGCTGCTGATCTATACCCTGTCATTCC |
| | | GGGCCTCAGGAGTGCCTGACCGCTTCTCGGGATCAGGGA |
| | | GCGGGACCGATTTCACCCTGAAAATTAGGCGAGTGGAAG |
| | | CCGAGGACGTCGGAGTGTACTACTGCATGCAGCGCATCG |
| | | GCTTCCCGATTACGTTTGGACAGGGTACCCGGCTTGAGAT |
| | | CAAG |
| SEQ ID | Full CAR | EVQLVESGGGLVKPGGSLRLSCAASGFTFSSFRMNWVRQAP |
| NO: | amino acid | GKGLEWVSSISSSSSYIYYADSVKGRFTISRDNAKNSLYLQM |
| 177 | sequence | NSLRAEDTAVYYCARWLSYYGMDVWGQGTTVTVSSGGGG |
| | | SGGGGSGGGGSGGGGSDIVMTQTPLSLPVTPGEPASISCRSS |
| | | QSLLDSDDGNTYLDWYLQKPGQSPQLLIYTLSFRASGVPDR |
| | | FSGSGSGTDFTLKIRRVEAEDVGVYYCMQRIGFPITFGQGTR |
| | | LEIKTTTPAPRPPTPAPTIASQPLSLRPEACRPAAGGAVHTRG |
| | | LDFACDIYIWAPLAGTCGVLLLSLVITLYCKRGRKKLLYIFK |
| | | QPFMRPVQTTQEEDGCSCRFPEEEEGGCELRVKFSRSADAP |
| | | AYQQGQNQLYNELNLGRREEYDVLDKRRGRDPEMGGKPR |
| | | RKNPQEGLYNELQKDKMAEAYSEIGMKGERRRGKGHDGL |
| | | YQGLSTATKDTYDALHMQALPPR |
| SEQ ID | Full CAR | GAAGTGCAACTGGTGGAGAGCGGTGGAGGGCTTGTCAAG |
| NO: | DNA | CCCGGAGGATCGCTGCGGCTGTCCTGTGCTGCGTCCGGGT |
| 178 | sequence | TCACCTTCTCCTCGTTCCGCATGAACTGGGTCAGACAGGC |
| | | ACCGGGAAAGGGCCTCGAATGGGTGTCCTCAATCTCATC |
| | | GTCCTCGTCCTACATCTACTACGCCGACTCCGTGAAAGGC |
| | | CGCTTCACCATCTCCCGGGACAACGCCAAGAACTCGCTGT |
| | | ACCTCCAAATGAATAGCCTCAGGGCGGAAGATACTGCTG |
| | | TGTATTACTGCGCACGCTGGCTTTCCTACTACGGCATGGA |
| | | CGTCTGGGGCCAAGGGACCACTGTGACCGTGTCTAGCGG |
| | | AGGCGGAGGTTCAGGGGGCGGTGGATCAGGCGGAGGAG |
| | | GATCGGGGGGTGGTGGATCGGATATCGTGATGACCCAGA |
| | | CTCCCCTGTCCCTGCCTGTGACTCCCGGAGAACCAGCCTC |
| | | CATTTCCTGCCGGTCCTCCCAGTCCCTGCTGGACAGCGAC |
| | | GACGGCAACACTTACCTGGACTGGTACTTGCAGAAGCCG |
| | | GGCCAATCGCCTCAGCTGCTGATCTATACCCTGTCATTCC |
| | | GGGCCTCAGGAGTGCCTGACCGCTTCTCGGGATCAGGGA |
| | | GCGGGACCGATTTCACCCTGAAAATTAGGCGAGTGGAAG |
| | | CCGAGGACGTCGGAGTGTACTACTGCATGCAGCGCATCG |
| | | GCTTCCCGATTACGTTTGGACAGGGTACCCGGCTTGAGAT |
| | | CAAGACCACTACCCCAGCACCGAGGCCACCCACCCCGGC |
| | | TCCTACCATCGCCTCCCAGCCTCTGTCCCTGCGTCCGGAG |
| | | GCATGTAGACCCGCAGCTGGTGGGGCCGTGCATACCCGG |
| | | GGTCTTGACTTCGCCTGCGATATCTACATTTGGGCCCCTC |
| | | TGGCTGGTACTTGCGGGGTCCTGCTGCTTTCACTCGTGAT |
| | | CACTCTTTACTGTAAGCGCGGTCGGAAGAAGCTGCTGTAC |
| | | ATCTTTAAGCAACCCTTCATGAGGCCTGTGCAGACTACTC |
| | | AAGAGGAGGACGGCTGTTCATGCCGGTTCCCAGAGGAGG |
| | | AGGAAGGCGGCTGCGAACTGCGCGTGAAATTCAGCCGCA |
| | | GCGCAGATGCTCCAGCCTACCAGCAGGGGCAGAACCAGC |
| | | TCTACAACGAACTCAATCTTGGTCGGAGAGAGGAGTACG |
| | | ACGTGCTGGACAAGCGGAGAGGACGGGACCCAGAAATG |
| | | GGCGGGAAGCCGCGCAGAAAGAATCCCCAAGAGGGCCT |
| | | GTACAACGAGCTCCAAAAGGATAAGATGGCAGAAGCCTA |
| | | TAGCGAGATTGGTATGAAAGGGGAACGCAGAAGAGGCA |
| | | AAGGCCACGACGGACTGTACCAGGGACTCAGCACCGCCA |
| | | CCAAGGACACCTATGACGCTCTTCACATGCAGGCCCTGCC |
| | | GCCTCGG |

TABLE 11
Kabat CDRs of exemplary hybridoma-derived anti-BCMA molecules

| Kabat | HCDR1 | HCDR2 | HCDR3 | LCDR1 | LCDR2 | LCDR3 |
|---|---|---|---|---|---|---|
| Hy03 | GFWMS | NIKQDGSEK | ALDYYGM | RSSQSLLD | TLSYRA | TQRLEF |
| | (SEQ ID | YYVDSVRG | DV (SEQ ID | SDDGNTY | S (SEQ | PSIT |
| | NO: 137) | (SEQ ID NO: | NO: 139) | LD (SEQ | ID NO: | (SEQ ID |
| | | 138) | | ID NO: | 148) | NO: 149) |
| | | | | 147) | | |
| Hy52 | SFRMN | SISSSSSYIY | WLSYYGM | RSSQSLLD | TLSFRA | MQRIGF |
| | (SEQ ID | YADSVKG | DV (SEQ ID | SDDGNTY | S (SEQ | PIT (SEQ |
| | NO: 160) | (SEQ ID NO: | NO: 162) | LD (SEQ | ID NO: | ID NO: |
| | | 161) | | ID NO: | 170) | 171) |
| | | | | 147) | | |
| Consensus | X₁FX₂MX₃, | X₁IX₂X₃X₄X₅ | X₁LX₂YYG | RSSQSLLD | TLSXRAS, | X₁QRX₂X₃F |
| | wherein | SX₆X₇YYX₈ | MDV, | SDDGNTY | wherein | PX₄IT, |
| | X₁ is G or | DSVX₉G, | wherein X₁ | LD (SEQ | X is Y or | wherein |
| | S; X₂ is W | wherein X₁ is | is A or W; | ID NO: | F (SEQ | X₁ is T or |
| | or R; and | N or S; X₂ is | and X₂ is | 147) | ID NO: | M; X₂ is |
| | X₃ is S or | K or S; X₃ is | D or S | | 182) | L or I; X₃ |
| | N (SEQ | Q or S; X₄ is | (SEQ ID | | | is E or G; |
| | ID NO: | D or S; X₅ is | NO: 181) | | | and X₄ is |
| | 179) | G or S; X₆ is | | | | S or |
| | | E or Y; X₇ is | | | | absent |
| | | K or I; X₈ is | | | | (SEQ ID |
| | | V or A; and | | | | NO: 183) |
| | | X₉ is R or K | | | | |
| | | (SEQ ID NO: | | | | |
| | | 180) | | | | |

TABLE 12
Chothia CDRs of exemplary hybridoma-derived anti-BCMA molecules

| Chothia | HCDR1 | HCDR2 | HCDR3 | LCDR1 | LCDR2 | LCDR3 |
|---|---|---|---|---|---|---|
| Hy03 | GFTFSGF | KQDGSE | ALDYYGM | SQSLLDS | TLS | RLEFPSI |
| | (SEQ ID | (SEQ ID NO: | DV (SEQ ID | DDGNTY | (SEQ | (SEQ ID |
| | NO: 140) | 141) | NO: 139) | (SEQ ID | ID NO: | NO: 152) |
| | | | | NO: 150) | 151) | |
| Hy52 | GFTFSSF | SSSSSY (SEQ | WLSYYGM | SQSLLDS | TLS | RIGFPI |
| | (SEQ ID | ID NO: 164) | DV (SEQ ID | DDGNTY | (SEQ | (SEQ ID |
| | NO: 163) | | NO: 162) | (SEQ ID | ID NO: | NO: 172) |
| | | | | NO: 150) | 151) | |
| Consensus | GFTFSXF, | X₁X₂X₃X₄SX₅, | X₁LX₂YYG | SQSLLDS | TLS | RX₁X₂FP |
| | wherein X | wherein X₁ is | MDV, | DDGNTY | (SEQ | X₃I, |
| | is G or S | K or S; X₂ is Q | wherein X₁ is | (SEQ ID | ID NO: | wherein |
| | (SEQ ID | or S; X₃ is D or | A or W; and | NO: 150) | 151) | X₁ is L or |
| | NO: 184) | S; X₄ is G or S; | X₂ is D or S | | | I; X₂ is E |
| | | and X₅ is E or | (SEQ ID NO: | | | or G; and |
| | | Y (SEQ ID | 181) | | | X₃ is S or |
| | | NO: 185) | | | | absent |
| | | | | | | (SEQ ID |
| | | | | | | NO: 186) |

TABLE 13
IMGT CDRs of exemplary hybridoma-derived anti-BCMA molecules

| IMGT | HCDR1 | HCDR2 | HCDR3 | LCDR1 | LCDR2 | LCDR3 |
|---|---|---|---|---|---|---|
| Hy03 | GFTFSGF | IKQDGSEK | ARALDYY | QSLLDSD | TLS | TQRLEFP |
| | W (SEQ ID | (SEQ ID | GMDV | DGNTY | (SEQ ID | SIT (SEQ |
| | NO: 142) | NO: 143) | (SEQ ID | (SEQ ID | NO: | ID NO: |
| | | | NO: 144) | NO: 153) | 151) | 149) |
| Hy52 | GFTFSSF | ISSSSSYI | ARWLSYY | QSLLDSD | TLS | MQRIGFP |
| | R (SEQ ID | (SEQ ID | GMDV | DGNTY | (SEQ ID | IT (SEQ |
| | NO: 165) | NO: 166) | (SEQ ID | (SEQ ID | NO: | ID NO: |
| | | | NO: 167) | NO: 153) | 151) | 171) |
| Consensus | GFTFSX₁F | IX₁X₂X₃X₄S | ARX₁LX₂Y | QSLLDSD | TLS | X₁QRX₂X₃ |
| | X₂, | X₅X₆, | YGMDV, | DGNTY | (SEQ ID | FPX₄IT, |
| | wherein X₁ | wherein X₁ is | wherein X₁ | (SEQ ID | NO: | wherein X₁ |
| | is G or S; | K or S; X₂ is | is A or W; | NO: 153) | 151) | is T or M; |
| | and X₂ is | Q or S; X₃ is | and X₂ is D | | | X₂ is L or |
| | W or R | D or S; X₄ is | or S | | | I; X₃ is E |
| | (SEQ ID | G or S; X₅ is | (SEQ ID | | | or G; and |
| | NO: 187) | E or Y; and | NO: 189) | | | X₄ is S or |
| | | X₆ is K or I | | | | absent |
| | | (SEQ ID | | | | (SEQ ID |
| | | NO: 188) | | | | NO: 183) |

In some embodiments, BCMA CARs may be generated using the VH and VL sequences from WO2012/0163805 (the contents of which are hereby incorporated by reference in its entirety). In some embodiments, BCMA CARs may be generated using the CDRs, VHs, VLs, scFvs, or full-CAR sequences from WO2019/241426 (the contents of which are hereby incorporated by reference in its entirety).

Other Exemplary Targets

Further non-limiting exemplary tumor antigens include CD20, CD22, EGFR, CD123, and CLL-1.

CARs that bind to CD20 are known in the art. For example, those disclosed in WO2018/067992 or WO2016/164731, incorporated by reference herein. Any known CD20 CAR, for example, the CD20 antigen binding domain of any known CD20 CAR, in the art can be used in accordance with the present disclosure. Exemplary CD20-binding sequences or CD20 CAR sequences are disclosed in, for example, Tables 1-5 of WO2018/067992, incorporated by reference. In some embodiments, the CD20 CAR comprises a CDR, variable region, scFv, or full-length sequence of a CD20 CAR disclosed in WO2018/067992 or WO2016/164731, both incorporated by reference herein. In some embodiments, CD20 CARs comprise a sequence, for example, a CDR, VH, VL, scFv, or full-CAR sequence, disclosed in Table 23 below, or a sequence having at least 70%, 75%, 80%, 85%, 90%, 95%, 96%, 97%, 98%, or 99% identity thereto.

TABLE 23
Amino acid sequences of exemplary anti-CD20 molecules

| SEQ ID NO: | Region | Sequence |
|---|---|---|
| CD20-C3H2 | | |
| 769 | HCDR1 | NYNLH |
| | (Kabat) | |
| 770 | HCDR2 | AIYPGNYDTSYNQKFKG |
| | (Kabat) | |
| 771 | HCDR3 | VDFGHSRYWYFDV |
| | (Kabat) | |
| 772 | HCDR1 | GYTFTNY |
| | (Chothia) | |
| 773 | HCDR2 | YPGNYD |
| | (Chothia) | |
| 771 | HCDR3 | VDFGHSRYWYFDV |
| | (Chothia) | |
| 774 | HCDR1 | GYTFTNYN |
| | (IMGT) | |
| 775 | HCDR2 | IYPGNYDT |
| | (IMGT) | |
| 776 | HCDR3 | ARVDFGHSRYWYFDV |
| | (IMGT) | |
| 777 | HCDR1 | GYTFTNYNLH |
| | (Combined) | |
| 770 | HCDR2 | AIYPGNYDTSYNQKFKG |
| | (Combined) | |
| 771 | HCDR3 | VDFGHSRYWYFDV |
| | (Combined) | |
| 778 | VH | QVQLVQSGAEVKKPGASVKVSCKASGYTFTNYNLHWVRQAP |
| | | GQGLEWMGAIYPGNYDTSYNQKFKGRVTMTADKSTSTAYME |
| | | LSSLRSEDTAVYYCARVDFGHSRYWYFDVWGQGTTVTVSS |
| 779 | DNA VH | CAAGTCCAACTCGTCCAGTCCGGTGCAGAAGTCAAGAAACC |
| | | TGGAGCATCCGTGAAAGTGTCTTGCAAAGCCTCCGGCTACA |
| | | CCTTCACCAACTACAACCTCCATTGGGTCAGACAGGCCCCC |
| | | GGACAAGGACTCGAATGGATGGGAGCGATCTACCCGGGAA |
| | | ACTACGACACCAGCTACAACCAGAAGTTCAAGGGCCGCGTG |
| | | ACTATGACCGCCGATAAGAGCACCTCCACCGCCTACATGGA |
| | | ACTGTCCTCGCTGAGGTCCGAGGACACTGCGGTGTACTACT |
| | | GCGCCCGCGTGGACTTCGGACACTCACGGTATTGGTACTTC |
| | | GACGTCTGGGGACAGGGCACTACCGTGACCGTGTCGAGC |
| 780 | LCDR1 | RATSSVSSMN |
| | (Kabat) | |
| 781 | LCDR2 | ATSNLAS |
| | (Kabat) | |
| 782 | LCDR3 | QQWTFNPPT |
| | (Kabat) | |
| 783 | LCDR1 | TSSVSS |
| | (Chothia) | |
| 784 | LCDR2 | ATS |
| | (Chothia) | |
| 785 | LCDR3 | WTFNPP |
| | (Chothia) | |
| 786 | LCDR1 | SSVSS |
| | (IMGT) | |
| 784 | LCDR2 | ATS |
| | (IMGT) | |
| 782 | LCDR3 | QQWTFNPPT |
| | (IMGT) | |
| 780 | LCDR1 | RATSSVSSMN |
| | (Combined) | |
| 781 | LCDR2 | ATSNLAS |
| | (Combined) | |
| 782 | LCDR3 | QQWTFNPPT |
| | (Combined) | |
| 787 | VL | DIQLTQSPSFLSASVGDRVTITCRATSSVSSMNWYQQKPGKAP |
| | | KPLIHATSNLASGVPSRFSGSGSGTEYTLTISSLQPEDFATYYCQ |
| | | QWTFNPPTFGQGTKLEIK |
| 788 | DNA VL | GATATCCAGCTGACTCAGTCCCCGTCATTCCTGTCCGCCTCC |
| | | GTGGGAGACAGAGTGACCATCACCTGTCGGGCCACTTCCTC |
| | | CGTGTCAAGCATGAACTGGTATCAGCAGAAGCCCGGGAAGG |
| | | CCCCAAAGCCGCTGATTCACGCGACGTCCAACCTGGCTTCC |
| | | GGCGTGCCGAGCCGGTTCTCCGGCTCGGGGAGCGGGACTGA |
| | | GTACACCCTGACTATTTCCTCGCTTCAACCCGAGGACTTTGC |
| | | TACCTACTACTGCCAACAGTGGACCTTCAATCCTCCGACATT |
| | | CGGACAGGGTACCAAGTTGGAAATCAAG |
| 789 | scFv (VH- | QVQLVQSGAEVKKPGASVKVSCKASGYTFTNYNLHWVRQAP |
| | linker-VL) | GQGLEWMGAIYPGNYDTSYNQKFKGRVTMTADKSTSTAYME |
| | | LSSLRSEDTAVYYCARVDFGHSRYWYFDVWGQGTTVTVSSGG |
| | | GGSGGGGSGGGGSGGGGSDIQLTQSPSFLSASVGDRVTITCRA |
| | | TSSVSSMNWYQQKPGKAPKPLIHATSNLASGVPSRFSGSGSGT |
| | | EYTLTISSLQPEDFATYYCQQWTFNPPTFGQGTKLEIK |
| 790 | DNA scFv | caagtccaactcgtccagtccggtgcagaagtcaagaaacctggagcatccgtgaaagtgtcttgc |
| | (VH-linker- | aaagcctccggctacaccttcaccaactacaacctccattgggtcagacaggcccccggacaagg |
| | VL) | actcgaatggatgggagcgatctacccgggaaactacgacaccagctacaaccagaagttcaagg |
| | | gccgcgtgactatgaccgccgataagagcacctccaccgcctacatggaactgtcctcgctgaggt |
| | | ccgaggacactgcggtgtactactgcgcccgcgtggacttcggacactcacggtattggtacttcga |
| | | cgtctggggacagggcactaccgtgaccgtgtcgagcggcggaggaggttcgggagggggcgg |
| | | atcagggggcggcggcagcggtggagggggctcggatatccagctgactcagtccccgtcattcc |
| | | tgtccgcctccgtgggagacagagtgaccatcacctgtcgggccacttcctccgtgtcaagcatgaa |
| | | ctggtatcagcagaagcccgggaaggccccaaagccgctgattcacgcgacgtccaacctggctt |
| | | ccggcgtgccgagccggttctccggctcggggagcgggactgagtacaccctgactatttcctcgc |
| | | ttcaacccgaggactttgctacctactactgccaacagtggaccttcaatcctccgacattcggacag |
| | | ggtaccaagttggaaatcaag |
| 791 | Full CAR | MALPVTALLLPLALLLHAARPQVQLVQSGAEVKKPGASVKVS |
| | amino acid | CKASGYTFTNYNLHWVRQAPGQGLEWMGAIYPGNYDTSYNQ |
| | sequence | KFKGRVTMTADKSTSTAYMELSSLRSEDTAVYYCARVDFGHS |
| | | RYWYFDVWGQGTTVTVSSGGGGSGGGGSGGGGSGGGGSDIQ |
| | | LTQSPSFLSASVGDRVTITCRATSSVSSMNWYQQKPGKAPKPLI |
| | | HATSNLASGVPSRFSGSGSGTEYTLTISSLQPEDFATYYCQQWT |
| | | FNPPTFGQGTKLEIKTTTPAPRPPTPAPTIASQPLSLRPEACRPAA |
| | | GGAVHTRGLDFACDIYIWAPLAGTCGVLLLSLVITLYCKRGRK |
| | | KLLYIFKQPFMRPVQTTQEEDGCSCRFPEEEEGGCELRVKFSRS |
| | | ADAPAYQQGQNQLYNELNLGRREEYDVLDKRRGRDPEMGGK |
| | | PRRKNPQEGLYNELQKDKMAEAYSEIGMKGERRRGKGHDGL |
| | | YQGLSTATKDTYDALHMQALPPR |
| 792 | Full CAR | ATGGCCCTCCCTGTCACCGCCCTGCTGCTTCCGCTGGCTCTT |
| | nucleotide | CTGCTCCACGCCGCTCGGCCCCAAGTCCAACTCGTCCAGTCC |
| | sequence | GGTGCAGAAGTCAAGAAACCTGGAGCATCCGTGAAAGTGTC |
| | | TTGCAAAGCCTCCGGCTACACCTTCACCAACTACAACCTCCA |
| | | TTGGGTCAGACAGGCCCCCGGACAAGGACTCGAATGGATGG |
| | | GAGCGATCTACCCGGGAAACTACGACACCAGCTACAACCAG |
| | | AAGTTCAAGGGCCGCGTGACTATGACCGCCGATAAGAGCAC |
| | | CTCCACCGCCTACATGGAACTGTCCTCGCTGAGGTCCGAGG |
| | | ACACTGCGGTGTACTACTGCGCCCGCGTGGACTTCGGACAC |
| | | TCACGGTATTGGTACTTCGACGTCTGGGGACAGGGCACTAC |
| | | CGTGACCGTGTCGAGCGGCGGAGGAGGTTCGGGAGGGGGC |
| | | GGATCAGGGGGCGGCGGCAGCGGTGGAGGGGGCTCGGATA |
| | | TCCAGCTGACTCAGTCCCCGTCATTCCTGTCCGCCTCCGTGG |
| | | GAGACAGAGTGACCATCACCTGTCGGGCCACTTCCTCCGTG |
| | | TCAAGCATGAACTGGTATCAGCAGAAGCCCGGGAAGGCCCC |
| | | AAAGCCGCTGATTCACGCGACGTCCAACCTGGCTTCCGGCG |
| | | TGCCGAGCCGGTTCTCCGGCTCGGGGAGCGGGACTGAGTAC |
| | | ACCCTGACTATTTCCTCGCTTCAACCCGAGGACTTTGCTACC |
| | | TACTACTGCCAACAGTGGACCTTCAATCCTCCGACATTCGGA |
| | | CAGGGTACCAAGTTGGAAATCAAGACCACTACCCCAGCACC |
| | | GAGGCCACCCACCCCGGCTCCTACCATCGCCTCCCAGCCTCT |
| | | GTCCCTGCGTCCGGAGGCATGTAGACCCGCAGCTGGTGGGG |
| | | CCGTGCATACCCGGGGTCTTGACTTCGCCTGCGATATCTACA |
| | | TTTGGGCCCCTCTGGCTGGTACTTGCGGGGTCCTGCTGCTTT |
| | | CACTCGTGATCACTCTTTACTGTAAGCGCGGTCGGAAGAAG |
| | | CTGCTGTACATCTTTAAGCAACCCTTCATGAGGCCTGTGCAG |
| | | ACTACTCAAGAGGAGGACGGCTGTTCATGCCGGTTCCCAGA |
| | | GGAGGAGGAAGGCGGCTGCGAACTGCGCGTGAAATTCAGC |
| | | CGCAGCGCAGATGCTCCAGCCTACCAGCAGGGGCAGAACCA |
| | | GCTCTACAACGAACTCAATCTTGGTCGGAGAGAGGAGTACG |
| | | ACGTGCTGGACAAGCGGAGAGGACGGGACCCAGAAATGGG |
| | | CGGGAAGCCGCGCAGAAAGAATCCCCAAGAGGGCCTGTAC |
| | | AACGAGCTCCAAAAGGATAAGATGGCAGAAGCCTATAGCG |
| | | AGATTGGTATGAAAGGGGAACGCAGAAGAGGCAAAGGCCA |
| | | CGACGGACTGTACCAGGGACTCAGCACCGCCACCAAGGACA |
| | | CCTATGACGCTCTTCACATGCAGGCCCTGCCGCCTCGG |
| CD20-C5H1 | | |
| 793 | HCDR1 | SYNMH |
| | (Kabat) | |
| 794 | HCDR2 | AIYPGNGDTSYNPKFKG |
| | (Kabat) | |
| 795 | HCDR3 | SYFYGSSSWYFDV |
| | (Kabat) | |
| 523 | HCDR1 | GYTFTSY |
| | (Chothia) | |
| 796 | HCDR2 | YPGNGD |
| | (Chothia) | |
| 795 | HCDR3 | SYFYGSSSWYFDV |
| | (Chothia) | |
| 797 | HCDR1 | GYTFTSYN |
| | (IMGT) | |
| 798 | HCDR2 | IYPGNGDT |
| | (IMGT) | |
| 799 | HCDR3 | ARSYFYGSSSWYFDV |
| | (IMGT) | |
| 800 | HCDR1 | GYTFTSYNMH |
| | (Combined) | |
| 794 | HCDR2 | AIYPGNGDTSYNPKFKG |
| | (Combined) | |
| 795 | HCDR3 | SYFYGSSSWYFDV |
| | (Combined) | |
| 801 | VH | QVQLVQSGAEVKKPGASVKVSCKASGYTFTSYNMHWVRQAP |
| | | GQGLEWMGAIYPGNGDTSYNPKFKGRVTMTADKSTRTAYME |
| | | LSSLRSEDTAVYYCARSYFYGSSSWYFDVWGQGTTVTVSS |
| 802 | DNA VH | CAAGTGCAGCTCGTCCAGTCCGGTGCAGAAGTCAAGAAACC |
| | | CGGTGCTTCAGTGAAAGTGTCCTGCAAGGCCTCCGGTTACA |
| | | CCTTCACCTCCTACAACATGCACTGGGTCCGCCAAGCCCCG |
| | | GGCCAGGGACTCGAATGGATGGGAGCCATCTACCCTGGCAA |
| | | CGGGGACACCTCATACAACCCTAAGTTCAAGGGCAGAGTGA |
| | | CCATGACTGCGGACAAGTCCACTAGAACAGCGTACATGGAG |
| | | CTGAGCAGCCTGCGGTCCGAGGATACTGCCGTGTACTACTG |
| | | CGCCCGCTCCTACTTCTACGGAAGCTCGTCGTGGTACTTCGA |
| | | TGTCTGGGGACAGGGCACCACTGTGACTGTGTCCTCC |
| 803 | LCDR1 | RASSSVSSMH |
| | (Kabat) | |
| 781 | LCDR2 | ATSNLAS |
| | (Kabat) | |
| 804 | LCDR3 | QQWIFNPPT |
| | (Kabat) | |
| 805 | LCDR1 | SSSVSS |
| | (Chothia) | |
| 784 | LCDR2 | ATS |
| | (Chothia) | |
| 806 | LCDR3 | WIFNPP |
| | (Chothia) | |
| 786 | LCDR1 | SSVSS |
| | (IMGT) | |
| 784 | LCDR2 | ATS |
| | (IMGT) | |
| 804 | LCDR3 | QQWIFNPPT |
| | (IMGT) | |
| 803 | LCDR1 | RASSSVSSMH |
| | (Combined) | |
| 781 | LCDR2 | ATSNLAS |
| | (Combined) | |
| 804 | LCDR3 | QQWIFNPPT |
| | (Combined) | |
| 807 | VL | EIVLTQSPATLSLSPGERATLSCRASSSVSSMHWYQQKPGQAPR |
| | | PLIFATSNLASGIPARFSGSGSGTDYTLTISSLEPEDAAVYYCQQ |
| | | WIFNPPTFGGGTKVEIK |
| 808 | DNA VL | GAAATTGTGCTGACTCAGAGCCCCGCCACCCTGAGCTTGTC |
| | | CCCCGGGGAAAGGGCAACGCTGTCATGCCGCGCCTCGTCAT |
| | | CCGTGTCCTCCATGCATTGGTACCAGCAGAAGCCGGGACAG |
| | | GCCCCTCGGCCGCTGATCTTCGCCACCTCCAATCTCGCTTCC |
| | | GGCATTCCGGCCCGGTTCTCGGGAAGCGGGTCGGGGACCGA |
| | | CTATACCCTGACCATCTCTAGCCTTGAACCTGAGGACGCCGC |
| | | GGTGTACTATTGTCAACAGTGGATCTTTAACCCCCCAACCTT |
| | | CGGTGGAGGCACCAAAGTGGAGATTAAG |
| 809 | scFv (VH- | QVQLVQSGAEVKKPGASVKVSCKASGYTFTSYNMHWVRQAP |
| | linker-VL) | GQGLEWMGAIYPGNGDTSYNPKFKGRVTMTADKSTRTAYME |
| | | LSSLRSEDTAVYYCARSYFYGSSSWYFDVWGQGTTVTVSSGG |
| | | GGSGGGGSGGGGSGGGGSEIVLTQSPATLSLSPGERATLSCRAS |
| | | SSVSSMHWYQQKPGQAPRPLIFATSNLASGIPARFSGSGSGTDY |
| | | TLTISSLEPEDAAVYYCQQWIFNPPTFGGGTKVEIK |
| 810 | DNA scFv | Caagtgcagctcgtccagtccggtgcagaagtcaagaaacccggtgcttcagtgaaagtgtcctgc |
| | (VH-linker- | aaggcctccggttacaccttcacctcctacaacatgcactgggtccgccaagccccgggccaggg |
| | VL) | actcgaatggatgggagccatctaccctggcaacggggacacctcatacaaccctaagttcaaggg |
| | | cagagtgaccatgactgcggacaagtccactagaacagcgtacatggagctgagcagcctgcggt |
| | | ccgaggatactgccgtgtactactgcgcccgctcctacttctacggaagctcgtcgtggtacttcgat |
| | | gtctggggacagggcaccactgtgactgtgtcctccggtggcggaggctcgggcggaggcggaa |
| | | gcggcggcgggggatcgggaggaggagggtccgaaattgtgctgactcagagccccgccaccc |
| | | tgagcttgtcccccggggaaagggcaacgctgtcatgccgcgcctcgtcatccgtgtcctccatgc |
| | | attggtaccagcagaagccgggacaggcccctcggccgctgatcttcgccacctccaatctcgctt |
| | | ccggcattccggcccggttctcgggaagcgggtcggggaccgactataccctgaccatctctagcc |
| | | ttgaacctgaggacgccgcggtgtactattgtcaacagtggatctttaaccccccaaccttcggtgga |
| | | ggcaccaaagtggagattaag |
| 811 | Full CAR | MALPVTALLLPLALLLHAARPQVQLVQSGAEVKKPGASVKVS |
| | amino acid | CKASGYTFTSYNMHWVRQAPGQGLEWMGAIYPGNGDTSYNP |
| | sequence | KFKGRVTMTADKSTRTAYMELSSLRSEDTAVYYCARSYFYGS |
| | | SSWYFDVWGQGTTVTVSSGGGGSGGGGSGGGGSGGGGSEIVL |
| | | TQSPATLSLSPGERATLSCRASSSVSSMHWYQQKPGQAPRPLIF |
| | | ATSNLASGIPARFSGSGSGTDYTLTISSLEPEDAAVYYCQQWIF |
| | | NPPTFGGGTKVEIKTTTPAPRPPTPAPTIASQPLSLRPEACRPAA |
| | | GGAVHTRGLDFACDIYIWAPLAGTCGVLLLSLVITLYCKRGRK |
| | | KLLYIFKQPFMRPVQTTQEEDGCSCRFPEEEEGGCELRVKFSRS |
| | | ADAPAYQQGQNQLYNELNLGRREEYDVLDKRRGRDPEMGGK |
| | | PRRKNPQEGLYNELQKDKMAEAYSEIGMKGERRRGKGHDGL |
| | | YQGLSTATKDTYDALHMQALPPR |
| 812 | Full CAR | ATGGCCCTCCCTGTCACCGCCCTGCTGCTTCCGCTGGCTCTT |
| | nucleotide | CTGCTCCACGCCGCTCGGCCCCAAGTGCAGCTCGTCCAGTCC |
| | sequence | GGTGCAGAAGTCAAGAAACCCGGTGCTTCAGTGAAAGTGTC |
| | | CTGCAAGGCCTCCGGTTACACCTTCACCTCCTACAACATGCA |
| | | CTGGGTCCGCCAAGCCCCGGGCCAGGGACTCGAATGGATGG |
| | | GAGCCATCTACCCTGGCAACGGGGACACCTCATACAACCCT |
| | | AAGTTCAAGGGCAGAGTGACCATGACTGCGGACAAGTCCAC |
| | | TAGAACAGCGTACATGGAGCTGAGCAGCCTGCGGTCCGAGG |
| | | ATACTGCCGTGTACTACTGCGCCCGCTCCTACTTCTACGGAA |
| | | GCTCGTCGTGGTACTTCGATGTCTGGGGACAGGGCACCACT |
| | | GTGACTGTGTCCTCCGGTGGCGGAGGCTCGGGCGGAGGCGG |
| | | AAGCGGCGGCGGGGGATCGGGAGGAGGAGGGTCCGAAATT |
| | | GTGCTGACTCAGAGCCCCGCCACCCTGAGCTTGTCCCCCGG |
| | | GGAAAGGGCAACGCTGTCATGCCGCGCCTCGTCATCCGTGT |
| | | CCTCCATGCATTGGTACCAGCAGAAGCCGGGACAGGCCCCT |
| | | CGGCCGCTGATCTTCGCCACCTCCAATCTCGCTTCCGGCATT |
| | | CCGGCCCGGTTCTCGGGAAGCGGGTCGGGGACCGACTATAC |
| | | CCTGACCATCTCTAGCCTTGAACCTGAGGACGCCGCGGTGT |
| | | ACTATTGTCAACAGTGGATCTTTAACCCCCCAACCTTCGGTG |
| | | GAGGCACCAAAGTGGAGATTAAGACCACTACCCCAGCACCG |
| | | AGGCCACCCACCCCGGCTCCTACCATCGCCTCCCAGCCTCTG |
| | | TCCCTGCGTCCGGAGGCATGTAGACCCGCAGCTGGTGGGGC |
| | | CGTGCATACCCGGGGTCTTGACTTCGCCTGCGATATCTACAT |
| | | TTGGGCCCCTCTGGCTGGTACTTGCGGGGTCCTGCTGCTTTC |
| | | ACTCGTGATCACTCTTTACTGTAAGCGCGGTCGGAAGAAGC |
| | | TGCTGTACATCTTTAAGCAACCCTTCATGAGGCCTGTGCAGA |
| | | CTACTCAAGAGGAGGACGGCTGTTCATGCCGGTTCCCAGAG |
| | | GAGGAGGAAGGCGGCTGCGAACTGCGCGTGAAATTCAGCC |
| | | GCAGCGCAGATGCTCCAGCCTACCAGCAGGGGCAGAACCA |
| | | GCTCTACAACGAACTCAATCTTGGTCGGAGAGAGGAGTACG |
| | | ACGTGCTGGACAAGCGGAGAGGACGGGACCCAGAAATGGG |
| | | CGGGAAGCCGCGCAGAAAGAATCCCCAAGAGGGCCTGTAC |
| | | AACGAGCTCCAAAAGGATAAGATGGCAGAAGCCTATAGCG |
| | | AGATTGGTATGAAAGGGGAACGCAGAAGAGGCAAAGGCCA |
| | | CGACGGACTGTACCAGGGACTCAGCACCGCCACCAAGGACA |
| | | CCTATGACGCTCTTCACATGCAGGCCCTGCCGCCTCGG |
| CD20-C3H1 | | |
| 896 | HCDR1 | NYNLH |
| | (Kabat) | |
| 897 | HCDR2 | AIYPGNYDTSYNQKFKG |
| | (Kabat) | |
| 898 | HCDR3 | VDFGHSRYWYFDV |
| | (Kabat) | |
| 899 | HCDR1 | GYTFTNY |
| | (Chothia) | |
| 900 | HCDR2 | YPGNYD |
| | (Chothia) | |
| 901 | HCDR3 | VDFGHSRYWYFDV |
| | (Chothia) | |
| 903 | HCDR1 | GYTFTNYN |
| | (IMGT) | |
| 904 | HCDR2 | IYPGNYDT |
| | (IMGT) | |
| 905 | HCDR3 | ARVDFGHSRYWYFDV |
| | (IMGT) | |
| 906 | HCDR1 | GYTFTNYNLH |
| | (Combined) | |
| 907 | HCDR2 | AIYPGNYDTSYNQKFKG |
| | (Combined) | |
| 908 | HCDR3 | VDFGHSRYWYFDV |
| | (Combined) | |
| 909 | VH | QVQLVQSGAEVKKPGASVKVSCKASGYTFTNYNLHWVRQAP |
| | | GQGLEWMGAIYPGNYDTSYNQKFKGRVTMTADKSTSTAYME |
| | | LSSLRSEDTAVYYCARVDFGHSRYWYFDVWGQGTTVTVSS |
| 910 | LCDR1 | RATSSVSSMN |
| | (Kabat) | |
| 911 | LCDR2 | ATSNLAS |
| | (Kabat) | |
| 912 | LCDR3 | QQWTFNPPT |
| | (Kabat) | |
| 913 | LCDR1 | TSSVSS |
| | (Chothia) | |
| 914 | LCDR2 | ATS |
| | (Chothia) | |
| 915 | LCDR3 | WTFNPP |
| | (Chothia) | |
| 916 | LCDR1 | SSVSS |
| | (IMGT) | |
| 917 | LCDR2 | ATS |
| | (IMGT) | |
| 918 | LCDR3 | QQWTFNPPT |
| | (IMGT) | |
| 919 | LCDR1 | RATSSVSSMN |
| | (Combined) | |
| 920 | LCDR2 | ATSNLAS |
| | (Combined) | |
| 921 | LCDR3 | QQWTFNPPT |
| | (Combined) | |
| 922 | VL | EIVLTQSPATLSLSPGERATLSCRATSSVSSMNWYQQKPGQAPR |
| | | PLIHATSNLASGIPARFSGSGSGTDYTLTISSLEPEDAAVYYCQQ |
| | | WTFNPPTFGQGTKLEIK |
| 923 | scFv (VH- | QVQLVQSGAEVKKPGASVKVSCKASGYTFTNYNLHWVRQAP |
| | linker-VL) | GQGLEWMGAIYPGNYDTSYNQKFKGRVTMTADKSTSTAYME |
| | | LSSLRSEDTAVYYCARVDFGHSRYWYFDVWGQGTTVTVSSGG |
| | | GGSGGGGSGGGGSGGGGSEIVLTQSPATLSLSPGERATLSCRAT |
| | | SSVSSMNWYQQKPGQAPRPLIHATSNLASGIPARFSGSGSGTD |
| | | YTLTISSLEPEDAAVYYCQQWTFNPPTFGQGTKLEIK |
| 924 | Full CAR | MALPVTALLLPLALLLHAARPQVQLVQSGAEVKKPGASVKVS |
| | | CKASGYTFTNYNLHWVRQAPGQGLEWMGAIYPGNYDTSYNQ |
| | | KFKGRVTMTADKSTSTAYMELSSLRSEDTAVYYCARVDFGHS |
| | | RYWYFDVWGQGTTVTVSSGGGGSGGGGSGGGGSGGGGSEIV |
| | | LTQSPATLSLSPGERATLSCRATSSVSSMNWYQQKPGQAPRPLI |
| | | HATSNLASGIPARFSGSGSGTDYTLTISSLEPEDAAVYYCQQWT |
| | | FNPPTFGQGTKLEIKTTTPAPRPPTPAPTIASQPLSLRPEACRPAA |
| | | GGAVHTRGLDFACDIYIWAPLAGTCGVLLLSLVITLYCKRGRK |
| | | KLLYIFKQPFMRPVQTTQEEDGCSCRFPEEEEGGCELRVKFSRS |
| | | ADAPAYQQGQNQLYNELNLGRREEYDVLDKRRGRDPEMGGK |
| | | PRRKNPQEGLYNELQKDKMAEAYSEIGMKGERRRGKGHDGL |
| | | YQGLSTATKDTYDALHMQALPPR |
| CD20-3H3 | | |
| 925 | HCDR1 | NYNLH |
| | (Kabat) | |
| 926 | HCDR2 | AIYPGNYDTSYNQKFKG |
| | (Kabat) | |
| 927 | HCDR3 | VDFGHSRYWYFDV |
| | (Kabat) | |
| 928 | HCDR1 | GYTFTNY |
| | (Chothia) | |
| 929 | HCDR2 | YPGNYD |
| | (Chothia) | |
| 930 | HCDR3 | VDFGHSRYWYFDV |
| | (Chothia) | |
| 931 | HCDR1 | GYTFTNYN |
| | (IMGT) | |
| 932 | HCDR2 | IYPGNYDT |
| | (IMGT) | |
| 933 | HCDR3 | ARVDFGHSRYWYFDV |
| | (IMGT) | |
| 934 | VH | QVQLVQSGAEVKKPGSSVKVSCKASGYTFTNYNLHWVRQAP |
| | | GQGLEWMGAIYPGNYDTSYNQKFKGRVTITADKSTSTAYMEL |
| | | SSLRSEDTAVYYCARVDFGHSRYWYFDVWGQGTTVTVSS |
| 935 | LCDR1 | RATSSVSSMN |
| | (Kabat) | |
| 936 | LCDR2 | ATSNLAS |
| | (Kabat) | |
| 937 | LCDR3 | QQWTFNPPT |
| | (Kabat) | |
| 938 | LCDR1 | TSSVSS |
| | (Chothia) | |
| 939 | LCDR2 | ATS |
| | (Chothia) | |
| 940 | LCDR3 | WTFNPP |
| | (Chothia) | |
| 941 | LCDR1 | SSVSS |
| | (IMGT) | |
| 942 | LCDR2 | ATS |
| | (IMGT) | |
| 943 | LCDR3 | QQWTFNPPT |
| | (IMGT) | |
| 944 | LCDR1 | RATSSVSSMN |
| | (Combined) | |
| 945 | LCDR2 | ATSNLAS |
| | (Combined) | |
| 946 | LCDR3 | QQWTFNPPT |
| | (Combined) | |
| 947 | VL | EIVLTQSPATLSLSPGERATLSCRATSSVSSMNWYQQKPGQAPR |
| | | PLIHATSNLASGIPARFSGSGSGTDYTLTISSLEPEDAAVYYCQQ |
| | | WTFNPPTFGQGTKLEIK |
| 948 | scFv (VH- | QVQLVQSGAEVKKPGSSVKVSCKASGYTFTNYNLHWVRQAP |
| | linker-VL) | GQGLEWMGAIYPGNYDTSYNQKFKGRVTITADKSTSTAYMEL |
| | | SSLRSEDTAVYYCARVDFGHSRYWYFDVWGQGTTVTVSSGG |
| | | GGSGGGGSGGGGSGGGGSEIVLTQSPATLSLSPGERATLSCRAT |
| | | SSVSSMNWYQQKPGQAPRPLIHATSNLASGIPARFSGSGSGTD |
| | | YTLTISSLEPEDAAVYYCQQWTFNPPTFGQGTKLEIK |
| 949 | Full CAR | MALPVTALLLPLALLLHAARPQVQLVQSGAEVKKPGSSVKVS |
| | | CKASGYTFTNYNLHWVRQAPGQGLEWMGAIYPGNYDTSYNQ |
| | | KFKGRVTITADKSTSTAYMELSSLRSEDTAVYYCARVDFGHSR |
| | | YWYFDVWGQGTTVTVSSGGGGSGGGGSGGGGSGGGGSEIVL |
| | | TQSPATLSLSPGERATLSCRATSSVSSMNWYQQKPGQAPRPLIH |
| | | ATSNLASGIPARFSGSGSGTDYTLTISSLEPEDAAVYYCQQWTF |
| | | NPPTFGQGTKLEIKTTTPAPRPPTPAPTIASQPLSLRPEACRPAA |
| | | GGAVHTRGLDFACDIYIWAPLAGTCGVLLLSLVITLYCKRGRK |
| | | KLLYIFKQPFMRPVQTTQEEDGCSCRFPEEEEGGCELRVKFSRS |
| | | ADAPAYQQGQNQLYNELNLGRREEYDVLDKRRGRDPEMGGK |
| | | PRRKNPQEGLYNELQKDKMAEAYSEIGMKGERRRGKGHDGL |
| | | YQGLSTATKDTYDALHMQALPPR |
| CD20-C3H4 | | |
| 950 | HCDR1 | NYNLH |
| | (Kabat) | |
| 951 | HCDR2 | AIYPGNYDTSYNQKFKG |
| | (Kabat) | |
| 952 | HCDR3 | VDFGHSRYWYFDV |
| | (Kabat) | |
| 953 | HCDR1 | GYTFTNY |
| | (Chothia) | |
| 954 | HCDR2 | YPGNYD |
| | (Chothia) | |
| 955 | HCDR3 | VDFGHSRYWYFDV |
| | (Chothia) | |
| 956 | HCDR1 | GYTFTNYN |
| | (IMGT) | |
| 957 | HCDR2 | IYPGNYDT |
| | (IMGT) | |
| 958 | HCDR3 | ARVDFGHSRYWYFDV |
| | (IMGT) | |
| 959 | HCDR1 | GYTFTNYNLH |
| | (Combined) | |
| 960 | HCDR2 | AIYPGNYDTSYNQKFKG |
| | (Combined) | |
| 961 | HCDR3 | VDFGHSRYWYFDV |
| | (Combined) | |
| 962 | VH | QVQLVQSGAEVKKPGSSVKVSCKASGYTFTNYNLHWVRQAP |
| | | GQGLEWMGAIYPGNYDTSYNQKFKGRVTITADKSTSTAYMEL |
| | | SSLRSEDTAVYYCARVDFGHSRYWYFDVWGQGTTVTVSS |
| 963 | LCDR1 | RATSSVSSMN |
| | (Kabat) | |
| 964 | LCDR2 | ATSNLAS |
| | (Kabat) | |
| 965 | LCDR3 | QQWTFNPPT |
| | (Kabat) | |
| 966 | LCDR1 | TSSVSS |
| | (Chothia) | |
| 967 | LCDR2 | ATS |
| | (Chothia) | |
| 968 | LCDR3 | WTFNPP |
| | (Chothia) | |
| 969 | LCDR1 | SSVSS |
| | (IMGT) | |
| 970 | LCDR2 | ATS |
| | (IMGT) | |
| 971 | LCDR3 | QQWTFNPPT |
| | (IMGT) | |
| 972 | LCDR1 | RATSSVSSMN |
| | (Combined) | |
| 973 | LCDR2 | ATSNLAS |
| | (Combined) | |
| 974 | LCDR3 | QQWTFNPPT |
| | (Combined) | |
| 975 | VL | DIQLTQSPSFLSASVGDRVTITCRATSSVSSMNWYQQKPGKAP |
| | | KPLIHATSNLASGVPSRFSGSGSGTEYTLTISSLQPEDFATYYCQ |
| | | QWTFNPPTFGQGTKLEIK |
| 976 | scFv (VH- | QVQLVQSGAEVKKPGSSVKVSCKASGYTFTNYNLHWVRQAP |
| | linker-VL) | GQGLEWMGAIYPGNYDTSYNQKFKGRVTITADKSTSTAYMEL |
| | | SSLRSEDTAVYYCARVDFGHSRYWYFDVWGQGTTVTVSSGG |
| | | GGSGGGGSGGGGSGGGGSDIQLTQSPSFLSASVGDRVTITCRA |
| | | TSSVSSMNWYQQKPGKAPKPLIHATSNLASGVPSRFSGSGSGT |
| | | EYTLTISSLQPEDFATYYCQQWTFNPPTFGQGTKLEIK |
| 977 | Full CAR | MALPVTALLLPLALLLHAARPQVQLVQSGAEVKKPGSSVKVS |
| | | CKASGYTFTNYNLHWVRQAPGQGLEWMGAIYPGNYDTSYNQ |
| | | KFKGRVTITADKSTSTAYMELSSLRSEDTAVYYCARVDFGHSR |
| | | YWYFDVWGQGTTVTVSSGGGGSGGGGSGGGGSGGGGSDIQL |
| | | TQSPSFLSASVGDRVTITCRATSSVSSMNWYQQKPGKAPKPLIH |
| | | ATSNLASGVPSRFSGSGSGTEYTLTISSLQPEDFATYYCQQWTF |
| | | NPPTFGQGTKLEIKTTTPAPRPPTPAPTIASQPLSLRPEACRPAA |
| | | GGAVHTRGLDFACDIYIWAPLAGTCGVLLLSLVITLYCKRGRK |
| | | KLLYIFKQPFMRPVQTTQEEDGCSCRFPEEEEGGCELRVKFSRS |
| | | ADAPAYQQGQNQLYNELNLGRREEYDVLDKRRGRDPEMGGK |
| | | PRRKNPQEGLYNELQKDKMAEAYSEIGMKGERRRGKGHDGL |
| | | YQGLSTATKDTYDALHMQALPPR |
| CD20-C2H1 | | |
| 978 | HCDR1 | NYWMH |
| | (Kabat) | |
| 979 | HCDR2 | FITPTTGYPEYNQKFKD |
| | (Kabat) | |
| 980 | HCDR3 | RKVGKGVYYALDY |
| | (Kabat) | |
| 981 | HCDR1 | GYTFTNY |
| | (Chothia) | |
| 982 | HCDR2 | TPTTGY |
| | (Chothia) | |
| 983 | HCDR3 | RKVGKGVYYALDY |
| | (Chothia) | |
| 984 | HCDR1 | GYTFTNYW |
| | (IMGT) | |
| 985 | HCDR2 | ITPTTGYP |
| | (IMGT) | |
| 986 | HCDR3 | ARRKVGKGVYYALDY |
| | (IMGT) | |
| 987 | HCDR1 | GYTFTNYWMH |
| | (Combined) | |
| 988 | HCDR2 | FITPTTGYPEYNQKFKD |
| | (Combined) | |
| 989 | HCDR3 | RKVGKGVYYALDY |
| | (Combined) | |
| 990 | VH | QVQLVQSGAEVKKPGASVKVSCKASGYTFTNYWMHWVRQA |
| | | PGQGLEWMGFITPTTGYPEYNQKFKDRVTMTADKSTSTAYME |
| | | LSSLRSEDTAVYYCARRKVGKGVYYALDYWGQGTTVTVSS |
| 991 | LCDR1 | RASGNIHNYLA |
| | (Kabat) | |
| 992 | LCDR2 | NTKTLAD |
| | (Kabat) | |
| 993 | LCDR3 | QHFWSSPWT |
| | (Kabat) | |
| 994 | LCDR1 | SGNIHNY |
| | (Chothia) | |
| 995 | LCDR2 | NTK |
| | (Chothia) | |
| 996 | LCDR3 | FWSSPW |
| | (Chothia) | |
| 997 | LCDR1 | GNIHNY |
| | (IMGT) | |
| 998 | LCDR2 | NTK |
| | (IMGT) | |
| 999 | LCDR3 | QHFWSSPWT |
| | (IMGT) | |
| 1000 | LCDR1 | RASGNIHNYLA |
| | (Combined) | |
| 1001 | LCDR2 | NTKTLAD |
| | (Combined) | |
| 1002 | LCDR3 | QHFWSSPWT |
| | (Combined) | |
| 1003 | VL | DIQMTQSPSSLSASVGDRVTITCRASGNIHNYLAWYQQKPGKV |
| | | PKLLIYNTKTLADGVPSRFSGSGSGTDYTLTISSLQPEDVATYY |
| | | CQHFWSSPWTFGGGTKVEIK |
| 1004 | scFv (VH- | QVQLVQSGAEVKKPGASVKVSCKASGYTFTNYWMHWVRQA |
| | linker-VL) | PGQGLEWMGFITPTTGYPEYNQKFKDRVTMTADKSTSTAYME |
| | | LSSLRSEDTAVYYCARRKVGKGVYYALDYWGQGTTVTVSSG |
| | | GGGSGGGGSGGGGSGGGGSDIQMTQSPSSLSASVGDRVTITCR |
| | | ASGNIHNYLAWYQQKPGKVPKLLIYNTKTLADGVPSRFSGSGS |
| | | GTDYTLTISSLQPEDVATYYCQHFWSSPWTFGGGTKVEIK |
| 1005 | Full CAR | MALPVTALLLPLALLLHAARPQVQLVQSGAEVKKPGASVKVS |
| | | CKASGYTFTNYWMHWVRQAPGQGLEWMGFITPTTGYPEYNQ |
| | | KFKDRVTMTADKSTSTAYMELSSLRSEDTAVYYCARRKVGKG |
| | | VYYALDYWGQGTTVTVSSGGGGSGGGGSGGGGSGGGGSDIQ |
| | | MTQSPSSLSASVGDRVTITCRASGNIHNYLAWYQQKPGKVPKL |
| | | LIYNTKTLADGVPSRFSGSGSGTDYTLTISSLQPEDVATYYCQH |
| | | FWSSPWTFGGGTKVEIKTTTPAPRPPTPAPTIASQPLSLRPEACR |
| | | PAAGGAVHTRGLDFACDIYIWAPLAGTCGVLLLSLVITLYCKR |
| | | GRKKLLYIFKQPFMRPVQTTQEEDGCSCRFPEEEEGGCELRVK |
| | | FSRSADAPAYQQGQNQLYNELNLGRREEYDVLDKRRGRDPEM |
| | | GGKPRRKNPQEGLYNELQKDKMAEAYSEIGMKGERRRGKGH |
| | | DGLYQGLSTATKDTYDALHMQALPPR |
| CD20-C2H2 | | |
| 1006 | HCDR1 | NYWMH |
| | (Kabat) | |
| 1007 | HCDR2 | FITPTTGYPEYNQKFKD |
| | (Kabat) | |
| 1008 | HCDR3 | RKVGKGVYYALDY |
| | (Kabat) | |
| 1009 | HCDR1 | GYTFTNY |
| | (Chothia) | |
| 1010 | HCDR2 | TPTTGY |
| | (Chothia) | |
| 1011 | HCDR3 | RKVGKGVYYALDY |
| | (Chothia) | |
| 1012 | HCDR1 | GYTFTNYW |
| | (IMGT) | |
| 1013 | HCDR2 | ITPTTGYP |
| | (IMGT) | |
| 1014 | HCDR3 | ARRKVGKGVYYALDY |
| | (IMGT) | |
| 1015 | HCDR1 | GYTFTNYWMH |
| | (Combined) | |
| 1016 | HCDR2 | FITPTTGYPEYNQKFKD |
| | (Combined) | |
| 1017 | HCDR3 | RKVGKGVYYALDY |
| | (Combined) | |
| 1018 | VH | QVQLVQSGAEVKKPGSSVKVSCKASGYTFTNYWMHWVRQAP |
| | | GQGLEWMGFITPTTGYPEYNQKFKDRVTITADKSTSTAYMELS |
| | | SLRSEDTAVYYCARRKVGKGVYYALDYWGQGTTVTVSS |
| 1019 | LCDR1 | RASGNIHNYLA |
| | (Kabat) | |
| 1020 | LCDR2 | NTKTLAD |
| | (Kabat) | |
| 1021 | LCDR3 | QHFWSSPWT |
| | (Kabat) | |
| 1022 | LCDR1 | SGNIHNY |
| | (Chothia) | |
| 1023 | LCDR2 | NTK |
| | (Chothia) | |
| 1024 | LCDR3 | FWSSPW |
| | (Chothia) | |
| 1025 | LCDR1 | GNIHNY |
| | (IMGT) | |
| 1026 | LCDR2 | NTK |
| | (IMGT) | |
| 1027 | LCDR3 | QHFWSSPWT |
| | (IMGT) | |
| 1028 | LCDR1 | RASGNIHNYLA |
| | (Combined) | |
| 1029 | LCDR2 | NTKTLAD |
| | (Combined) | |
| 1030 | LCDR3 | QHFWSSPWT |
| | (Combined) | |
| 1031 | VL | DIQMTQSPSSLSASVGDRVTITCRASGNIHNYLAWYQQKPGKV |
| | | PKLLIYNTKTLADGVPSRFSGSGSGTDYTLTISSLQPEDVATYY |
| | | CQHFWSSPWTFGGGTKVEIK |
| 1032 | scFv (VH- | QVQLVQSGAEVKKPGSSVKVSCKASGYTFTNYWMHWVRQAP |
| | linker-VL) | GQGLEWMGFITPTTGYPEYNQKFKDRVTITADKSTSTAYMELS |
| | | SLRSEDTAVYYCARRKVGKGVYYALDYWGQGTTVTVSSGGG |
| | | GSGGGGSGGGGSGGGGSDIQMTQSPSSLSASVGDRVTITCRAS |
| | | GNIHNYLAWYQQKPGKVPKLLIYNTKTLADGVPSRFSGSGSGT |
| | | DYTLTISSLQPEDVATYYCQHFWSSPWTFGGGTKVEIK |
| 1033 | Full CAR | MALPVTALLLPLALLLHAARPQVQLVQSGAEVKKPGSSVKVS |
| | | CKASGYTFTNYWMHWVRQAPGQGLEWMGFITPTTGYPEYNQ |
| | | KFKDRVTITADKSTSTAYMELSSLRSEDTAVYYCARRKVGKG |
| | | VYYALDYWGQGTTVTVSSGGGGSGGGGSGGGGSGGGGSDIQ |
| | | MTQSPSSLSASVGDRVTITCRASGNIHNYLAWYQQKPGKVPKL |
| | | LIYNTKTLADGVPSRFSGSGSGTDYTLTISSLQPEDVATYYCQH |
| | | FWSSPWTFGGGTKVEIKTTTPAPRPPTPAPTIASQPLSLRPEACR |
| | | PAAGGAVHTRGLDFACDIYIWAPLAGTCGVLLLSLVITLYCKR |
| | | GRKKLLYIFKQPFMRPVQTTQEEDGCSCRFPEEEEGGCELRVK |
| | | FSRSADAPAYQQGQNQLYNELNLGRREEYDVLDKRRGRDPEM |
| | | GGKPRRKNPQEGLYNELQKDKMAEAYSEIGMKGERRRGKGH |
| | | DGLYQGLSTATKDTYDALHMQALPPR |
| CD20-C2H3 | | |
| 1034 | HCDR1 | NYWMH |
| | (Kabat) | |
| 1035 | HCDR2 | FITPTTGYPEYNQKFKD |
| | (Kabat) | |
| 1036 | HCDR3 | RKVGKGVYYALDY |
| | (Kabat) | |
| 1037 | HCDR1 | GYTFTNY |
| | (Chothia) | |
| 1038 | HCDR2 | TPTTGY |
| | (Chothia) | |
| 1039 | HCDR3 | RKVGKGVYYALDY |
| | (Chothia) | |
| 1040 | HCDR1 | GYTFTNYW |
| | (IMGT) | |
| 1041 | HCDR2 | ITPTTGYP |
| | (IMGT) | |
| 1042 | HCDR3 | ARRKVGKGVYYALDY |
| | (IMGT) | |
| 1043 | HCDR1 | GYTFTNYWMH |
| | (Combined) | |
| 1044 | HCDR2 | FITPTTGYPEYNQKFKD |
| | (Combined) | |
| 1045 | HCDR3 | RKVGKGVYYALDY |
| | (Combined) | |
| 1046 | VH | QVQLVQSGAEVKKPGASVKVSCKASGYTFTNYWMHWVRQA |
| | | PGQGLEWMGFITPTTGYPEYNQKFKDRVTMTADKSTSTAYME |
| | | LSSLRSEDTAVYYCARRKVGKGVYYALDYWGQGTTVTVSS |
| 1047 | LCDR1 | RASGNIHNYLA |
| | (Kabat) | |
| 1048 | LCDR2 | NTKTLAD |
| | (Kabat) | |
| 1049 | LCDR3 | QHFWSSPWT |
| | (Kabat) | |
| 1050 | LCDR1 | SGNIHNY |
| | (Chothia) | |
| 1051 | LCDR2 | NTK |
| | (Chothia) | |
| 1052 | LCDR3 | FWSSPW |
| | (Chothia) | |
| 1053 | LCDR1 | GNIHNY |
| | (IMGT) | |
| 1054 | LCDR2 | NTK |
| | (IMGT) | |
| 1055 | LCDR3 | QHFWSSPWT |
| | (IMGT) | |
| 1056 | LCDR1 | RASGNIHNYLA |
| | (Combined) | |
| 1057 | LCDR2 | NTKTLAD |
| | (Combined) | |
| 1058 | LCDR3 | QHFWSSPWT |
| | (Combined) | |
| 1059 | VL | AIRMTQSPFSLSASVGDRVTITCRASGNIHNYLAWYQQKPAKA |
| | | PKLFIYNTKTLADGVPSRFSGSGSGTDYTLTISSLQPEDFATYYC |
| | | QHFWSSPWTFGGGTKVEIK |
| 1060 | scFv (VH- | QVQLVQSGAEVKKPGASVKVSCKASGYTFTNYWMHWVRQA |
| | linker-VL) | PGQGLEWMGFITPTTGYPEYNQKFKDRVTMTADKSTSTAYME |
| | | LSSLRSEDTAVYYCARRKVGKGVYYALDYWGQGTTVTVSSG |
| | | GGGSGGGGSGGGGSGGGGSAIRMTQSPFSLSASVGDRVTITCR |
| | | ASGNIHNYLAWYQQKPAKAPKLFIYNTKTLADGVPSRFSGSGS |
| | | GTDYTLTISSLQPEDFATYYCQHFWSSPWTFGGGTKVEIK |
| 1061 | Full CAR | MALPVTALLLPLALLLHAARPQVQLVQSGAEVKKPGASVKVS |
| | | CKASGYTFTNYWMHWVRQAPGQGLEWMGFITPTTGYPEYNQ |
| | | KFKDRVTMTADKSTSTAYMELSSLRSEDTAVYYCARRKVGKG |
| | | VYYALDYWGQGTTVTVSSGGGGSGGGGSGGGGSGGGGSAIR |
| | | MTQSPFSLSASVGDRVTITCRASGNIHNYLAWYQQKPAKAPKL |
| | | FIYNTKTLADGVPSRFSGSGSGTDYTLTISSLQPEDFATYYCQH |
| | | FWSSPWTFGGGTKVEIKTTTPAPRPPTPAPTIASQPLSLRPEACR |
| | | PAAGGAVHTRGLDFACDIYIWAPLAGTCGVLLLSLVITLYCKR |
| | | GRKKLLYIFKQPFMRPVQTTQEEDGCSCRFPEEEEGGCELRVK |
| | | FSRSADAPAYQQGQNQLYNELNLGRREEYDVLDKRRGRDPEM |
| | | GGKPRRKNPQEGLYNELQKDKMAEAYSEIGMKGERRRGKGH |
| | | DGLYQGLSTATKDTYDALHMQALPPR |
| CD20-C2H4 | | |
| 1062 | HCDR1 | NYWMH |
| | (Kabat) | |
| 1063 | HCDR2 | FITPTTGYPEYNQKFKD |
| | (Kabat) | |
| 1064 | HCDR3 | RKVGKGVYYALDY |
| | (Kabat) | |
| 1065 | HCDR1 | GYTFTNY |
| | (Chothia) | |
| 1066 | HCDR2 | TPTTGY |
| | (Chothia) | |
| 1067 | HCDR3 | RKVGKGVYYALDY |
| | (Chothia) | |
| 1068 | HCDR1 | GYTFTNYW |
| | (IMGT | |
| 1069 | HCDR2 | ITPTTGYP |
| | (IMGT) | |
| 1070 | HCDR3 | ARRKVGKGVYYALDY |
| | (IMGT) | |
| 1071 | HCDR1 | GYTFTNYWMH |
| | (Combined) | |
| 1072 | HCDR2 | FITPTTGYPEYNQKFKD |
| | (Combined) | |
| 1073 | HCDR3 | RKVGKGVYYALDY |
| | (Combined) | |
| 1074 | VH | QVQLVQSGAEVKKPGSSVKVSCKASGYTFTNYWMHWVRQAP |
| | | GQGLEWMGFITPTTGYPEYNQKFKDRVTITADKSTSTAYMELS |
| | | SLRSEDTAVYYCARRKVGKGVYYALDYWGQGTTVTVSS |
| 1075 | LCDR1 | RASGNIHNYLA |
| | (Kabat) | |
| 1076 | LCDR2 | NTKTLAD |
| | (Kabat) | |
| 1077 | LCDR3 | QHFWSSPWT |
| | (Kabat) | |
| 1078 | LCDR1 | SGNIHNY |
| | (Chothia) | |
| 1079 | LCDR2 | NTK |
| | (Chothia) | |
| 1080 | LCDR3 | FWSSPW |
| | (Chothia) | |
| 1081 | LCDR1 | GNIHNY |
| | (IMGT) | |
| 1082 | LCDR2 | NTK |
| | (IMGT) | |
| 1083 | LCDR3 | QHFWSSPWT |
| | (IMGT) | |
| 1084 | LCDR1 | RASGNIHNYLA |
| | (Combined) | |
| 1085 | LCDR2 | NTKTLAD |
| | (Combined) | |
| 1086 | LCDR3 | QHFWSSPWT |
| | (Combined) | |
| 1087 | VL | AIRMTQSPFSLSASVGDRVTITCRASGNIHNYLAWYQQKPAKA |
| | | PKLFIYNTKTLADGVPSRFSGSGSGTDYTLTISSLQPEDFATYYC |
| | | QHFWSSPWTFGGGTKVEIK |
| 1088 | scFv (VH- | QVQLVQSGAEVKKPGSSVKVSCKASGYTFTNYWMHWVRQAP |
| | linker-VL) | GQGLEWMGFITPTTGYPEYNQKFKDRVTITADKSTSTAYMELS |
| | | SLRSEDTAVYYCARRKVGKGVYYALDYWGQGTTVTVSSGGG |
| | | GSGGGGSGGGGSGGGGSAIRMTQSPFSLSASVGDRVTITCRAS |
| | | GNIHNYLAWYQQKPAKAPKLFIYNTKTLADGVPSRFSGSGSGT |
| | | DYTLTISSLQPEDFATYYCQHFWSSPWTFGGGTKVEIK |
| 1089 | Full CAR | MALPVTALLLPLALLLHAARPQVQLVQSGAEVKKPGSSVKVS |
| | | CKASGYTFTNYWMHWVRQAPGQGLEWMGFITPTTGYPEYNQ |
| | | KFKDRVTITADKSTSTAYMELSSLRSEDTAVYYCARRKVGKG |
| | | VYYALDYWGQGTTVTVSSGGGGSGGGGSGGGGSGGGGSAIR |
| | | MTQSPFSLSASVGDRVTITCRASGNIHNYLAWYQQKPAKAPKL |
| | | FIYNTKTLADGVPSRFSGSGSGTDYTLTISSLQPEDFATYYCQH |
| | | FWSSPWTFGGGTKVEIKTTTPAPRPPTPAPTIASQPLSLRPEACR |
| | | PAAGGAVHTRGLDFACDIYIWAPLAGTCGVLLLSLVITLYCKR |
| | | GRKKLLYIFKQPFMRPVQTTQEEDGCSCRFPEEEEGGCELRVK |
| | | FSRSADAPAYQQGQNQLYNELNLGRREEYDVLDKRRGRDPEM |
| | | GGKPRRKNPQEGLYNELQKDKMAEAYSEIGMKGERRRGKGH |
| | | DGLYQGLSTATKDTYDALHMQALPPR |
| CD20-C5H2 | | |
| 1090 | HCDR1 | SYNMH |
| | (Kabat) | |
| 1091 | HCDR2 | AIYPGNGDTSYNPKFKG |
| | (Kabat) | |
| 1092 | HCDR3 | SYFYGSSSWYFDV |
| | (Kabat) | |
| 1093 | HCDR1 | GYTFTSY |
| | (Chothia) | |
| 1094 | HCDR2 | YPGNGD |
| | (Chothia) | |
| 1095 | HCDR3 | SYFYGSSSWYFDV |
| | (Chothia) | |
| 1096 | HCDR1 | GYTFTSYN |
| | (IMGT) | |
| 1097 | HCDR2 | TYPGNGDT |
| | (IMGT) | |
| 1098 | HCDR3 | ARSYFYGSSSWYFDV |
| | (IMGT) | |
| 1099 | HCDR1 | GYTFTSYNMH |
| | (Combined) | |
| 1100 | HCDR2 | AIYPGNGDTSYNPKFKG |
| | (Combined) | |
| 1101 | HCDR3 | SYFYGSSSWYFDV |
| | (Combined) | |
| 1102 | VH | QVQLVQSGAEVKKPGASVKVSCKASGYTFTSYNMHWVRQAP |
| | | GQGLEWMGAIYPGNGDTSYNPKFKGRVTMTADKSTRTAYME |
| | | LSSLRSEDTAVYYCARSYFYGSSSWYFDVWGQGTTVTVSS |
| 1103 | LCDR1 | RASSSVSSMH |
| | (Kabat) | |
| 1104 | LCDR2 | ATSNLAS |
| | (Kabat) | |
| 1105 | LCDR3 | QQWIFNPPT |
| | (Kabat) | |
| 1106 | LCDR1 | SSSVSS |
| | (Chothia) | |
| 1107 | LCDR2 | ATS |
| | (Chothia) | |
| 1108 | LCDR3 | WIFNPP |
| | (Chothia) | |
| 1109 | LCDR1 | SSVSS |
| | (IMGT) | |
| 1110 | LCDR2 | ATS |
| | (IMGT) | |
| 1111 | LCDR3 | QQWIFNPPT |
| | (IMGT) | |
| 1112 | LCDR1 | RASSSVSSMH |
| | (Combined) | |
| 1113 | LCDR2 | ATSNLAS |
| | (Combined) | |
| 1114 | LCDR3 | QQWIFNPPT |
| | (Combined) | |
| 1115 | VL | DIQLTQSPSFLSASVGDRVTITCRASSSVSSMHWYQQKPGKAP |
| | | KPLIFATSNLASGVPSRFSGSGSGTEYTLTISSLQPEDFATYYCQ |
| | | QWIFNPPTFGGGTKVEIK |
| 1116 | scFv (VH- | QVQLVQSGAEVKKPGASVKVSCKASGYTFTSYNMHWVRQAP |
| | linker-VL) | GQGLEWMGAIYPGNGDTSYNPKFKGRVTMTADKSTRTAYME |
| | | LSSLRSEDTAVYYCARSYFYGSSSWYFDVWGQGTTVTVSSGG |
| | | GGSGGGGSGGGGSGGGGSDIQLTQSPSFLSASVGDRVTITCRAS |
| | | SSVSSMHWYQQKPGKAPKPLIFATSNLASGVPSRFSGSGSGTE |
| | | YTLTISSLQPEDFATYYCQQWIFNPPTFGGGTKVEIK |
| 1117 | Full CAR | MALPVTALLLPLALLLHAARPQVQLVQSGAEVKKPGASVKVS |
| | | CKASGYTFTSYNMHWVRQAPGQGLEWMGAIYPGNGDTSYNP |
| | | KFKGRVTMTADKSTRTAYMELSSLRSEDTAVYYCARSYFYGS |
| | | SSWYFDVWGQGTTVTVSSGGGGSGGGGSGGGGSGGGGSDIQL |
| | | TQSPSFLSASVGDRVTITCRASSSVSSMHWYQQKPGKAPKPLIF |
| | | ATSNLASGVPSRFSGSGSGTEYTLTISSLQPEDFATYYCQQWIF |
| | | NPPTFGGGTKVEIKTTTPAPRPPTPAPTIASQPLSLRPEACRPAA |
| | | GGAVHTRGLDFACDIYIWAPLAGTCGVLLLSLVITLYCKRGRK |
| | | KLLYIFKQPFMRPVQTTQEEDGCSCRFPEEEEGGCELRVKFSRS |
| | | ADAPAYQQGQNQLYNELNLGRREEYDVLDKRRGRDPEMGGK |
| | | PRRKNPQEGLYNELQKDKMAEAYSEIGMKGERRRGKGHDGL |
| | | YQGLSTATKDTYDALHMQALPPR |
| CD20-C5H3 | | |
| 1118 | HCDR1 | SYNMH |
| | (Kabat) | |
| 1119 | HCDR2 | AIYPGNGDTSYNPKFKG |
| | (Kabat) | |
| 1120 | HCDR3 | SYFYGSSSWYFDV |
| | (Kabat) | |
| 1121 | HCDR1 | GYTFTSY |
| | (Chothia) | |
| 1122 | HCDR2 | YPGNGD |
| | (Chothia) | |
| 1123 | HCDR3 | SYFYGSSSWYFDV |
| | (Chothia) | |
| 1124 | HCDR1 | GYTFTSYN |
| | (IMGT) | |
| 1125 | HCDR2 | IYPGNGDT |
| | (IMGT) | |
| 1126 | HCDR3 | ARSYFYGSSSWYFDV |
| | (IMGT) | |
| 1127 | HCDR1 | GYTFTSYNMH |
| | (Combined) | |
| 1128 | HCDR2 | AIYPGNGDTSYNPKFKG |
| | (Combined) | |
| 1129 | HCDR3 | SYFYGSSSWYFDV |
| | (Combined) | |
| 1130 | VH | QVQLVQSGAEVKKPGSSVKVSCKASGYTFTSYNMHWVRQAP |
| | | GQGLEWMGAIYPGNGDTSYNPKFKGRVTITADKSTRTAYMEL |
| | | SSLRSEDTAVYYCARSYFYGSSSWYFDVWGQGTTVTVSS |
| 1131 | LCDR1 | RASSSVSSMH |
| | (Kabat) | |
| 1132 | LCDR2 | ATSNLAS |
| | (Kabat) | |
| 1133 | LCDR3 | QQWIFNPPT |
| | (Kabat) | |
| 1134 | LCDR1 | SSSVSS |
| | (Chothia) | |
| 1135 | LCDR2 | ATS |
| | (Chothia) | |
| 1136 | LCDR3 | WIFNPP |
| | (Chothia) | |
| 1137 | LCDR1 | SSVSS |
| | (IMGT) | |
| 1138 | LCDR2 | ATS |
| | (IMGT) | |
| 1139 | LCDR3 | QQWIFNPPT |
| | (IMGT) | |
| 1140 | LCDR1 | RASSSVSSMH |
| | (Combined) | |
| 1141 | LCDR2 | ATSNLAS |
| | (Combined) | |
| 1142 | LCDR3 | QQWIFNPPT |
| | (Combined) | |
| 1143 | VL | EIVLTQSPATLSLSPGERATLSCRASSSVSSMHWYQQKPGQAPR |
| | | PLIFATSNLASGIPARFSGSGSGTDYTLTISSLEPEDAAVYYCQQ |
| | | WIFNPPTFGGGTKVEIK |
| 1144 | scFv (VH- | QVQLVQSGAEVKKPGSSVKVSCKASGYTFTSYNMHWVRQAP |
| | linker-VL) | GQGLEWMGAIYPGNGDTSYNPKFKGRVTITADKSTRTAYMEL |
| | | SSLRSEDTAVYYCARSYFYGSSSWYFDVWGQGTTVTVSSGGG |
| | | GSGGGGSGGGGSGGGGSEIVLTQSPATLSLSPGERATLSCRASS |
| | | SVSSMHWYQQKPGQAPRPLIFATSNLASGIPARFSGSGSGTDYT |
| | | LTISSLEPEDAAVYYCQQWIFNPPTFGGGTKVEIK |
| 1145 | Full CAR | MALPVTALLLPLALLLHAARPQVQLVQSGAEVKKPGSSVKVS |
| | | CKASGYTFTSYNMHWVRQAPGQGLEWMGAIYPGNGDTSYNP |
| | | KFKGRVTITADKSTRTAYMELSSLRSEDTAVYYCARSYFYGSS |
| | | SWYFDVWGQGTTVTVSSGGGGSGGGGSGGGGSGGGGSEIVLT |
| | | QSPATLSLSPGERATLSCRASSSVSSMHWYQQKPGQAPRPLIFA |
| | | TSNLASGIPARFSGSGSGTDYTLTISSLEPEDAAVYYCQQWIFNP |
| | | PTFGGGTKVEIKTTTPAPRPPTPAPTIASQPLSLRPEACRPAAGG |
| | | AVHTRGLDFACDIYIWAPLAGTCGVLLLSLVITLYCKRGRKKL |
| | | LYIFKQPFMRPVQTTQEEDGCSCRFPEEEEGGCELRVKFSRSAD |
| | | APAYQQGQNQLYNELNLGRREEYDVLDKRRGRDPEMGGKPR |
| | | RKNPQEGLYNELQKDKMAEAYSEIGMKGERRRGKGHDGLYQ |
| | | GLSTATKDTYDALHMQALPPR |
| CD20-C5H4 | | |
| 1146 | HCDR1 | SYNMH |
| | (Kabat) | |
| 1147 | HCDR2 | AIYPGNGDTSYNPKFKG |
| | (Kabat) | |
| 1148 | HCDR3 | SYFYGSSSWYFDV |
| | (Kabat) | |
| 1149 | HCDR1 | GYTFTSY |
| | (Chothia) | |
| 1150 | HCDR2 | YPGNGD |
| | (Chothia) | |
| 1151 | HCDR3 | SYFYGSSSWYFDV |
| | (Chothia) | |
| 1152 | HCDR1 | GYTFTSYN |
| | (IMGT) | |
| 1153 | HCDR2 | IYPGNGDT |
| | (IMGT) | |
| 1154 | HCDR3 | ARSYFYGSSSWYFDV |
| | (IMGT) | |
| 1155 | HCDR1 | GYTFTSYNMH |
| | (Combined) | |
| 1156 | HCDR2 | AIYPGNGDTSYNPKFKG |
| | (Combined) | |
| 1157 | HCDR3 | SYFYGSSSWYFDV |
| | (Combined) | |
| 1158 | VH | QVQLVQSGAEVKKPGSSVKVSCKASGYTFTSYNMHWVRQAP |
| | | GQGLEWMGAIYPGNGDTSYNPKFKGRVTITADKSTRTAYMEL |
| | | SSLRSEDTAVYYCARSYFYGSSSWYFDVWGQGTTVTVSS |
| 1159 | LCDR1 | RASSSVSSMH |
| | (Kabat) | |
| 1160 | LCDR2 | ATSNLAS |
| | (Kabat) | |
| 1161 | LCDR3 | QQWIFNPPT |
| | (Kabat) | |
| 1162 | LCDR1 | SSSVSS |
| | (Chothia) | |
| 1163 | LCDR2 | ATS |
| | (Chothia) | |
| 1164 | LCDR3 | WIFNPP |
| | (Chothia) | |
| 1165 | LCDR1 | SSVSS |
| | (IMGT) | |
| 1166 | LCDR2 | ATS |
| | (IMGT) | |
| 1167 | LCDR3 | QQWIFNPPT |
| | (IMGT) | |
| 1168 | LCDR1 | RASSSVSSMH |
| | (Combined) | |
| 1169 | LCDR2 | ATSNLAS |
| | (Combined) | |
| 1170 | LCDR3 | QQWIFNPPT |
| | (Combined) | |
| 1171 | VL | DIQLTQSPSFLSASVGDRVTITCRASSSVSSMHWYQQKPGKAP |
| | | KPLIFATSNLASGVPSRFSGSGSGTEYTLTISSLQPEDFATYYCQ |
| | | QWIFNPPTFGGGTKVEIK |
| 1172 | scFv (VH- | QVQLVQSGAEVKKPGSSVKVSCKASGYTFTSYNMHWVRQAP |
| | linker-VL) | GQGLEWMGAIYPGNGDTSYNPKFKGRVTITADKSTRTAYMEL |
| | | SSLRSEDTAVYYCARSYFYGSSSWYFDVWGQGTTVTVSSGGG |
| | | GSGGGGSGGGGSGGGGSDIQLTQSPSFLSASVGDRVTITCRASS |
| | | SVSSMHWYQQKPGKAPKPLIFATSNLASGVPSRFSGSGSGTEY |
| | | TLTISSLQPEDFATYYCQQWIFNPPTFGGGTKVEIK |
| 1173 | Full CAR | MALPVTALLLPLALLLHAARPQVQLVQSGAEVKKPGSSVKVS |
| | | CKASGYTFTSYNMHWVRQAPGQGLEWMGAIYPGNGDTSYNP |
| | | KFKGRVTITADKSTRTAYMELSSLRSEDTAVYYCARSYFYGSS |
| | | SWYFDVWGQGTTVTVSSGGGGSGGGGSGGGGSGGGGSDIQL |
| | | TQSPSFLSASVGDRVTITCRASSSVSSMHWYQQKPGKAPKPLIF |
| | | ATSNLASGVPSRFSGSGSGTEYTLTISSLQPEDFATYYCQQWIF |
| | | NPPTFGGGTKVEIKTTTPAPRPPTPAPTIASQPLSLRPEACRPAA |
| | | GGAVHTRGLDFACDIYIWAPLAGTCGVLLLSLVITLYCKRGRK |
| | | KLLYIFKQPFMRPVQTTQEEDGCSCRFPEEEEGGCELRVKFSRS |
| | | ADAPAYQQGQNQLYNELNLGRREEYDVLDKRRGRDPEMGGK |
| | | PRRKNPQEGLYNELQKDKMAEAYSEIGMKGERRRGKGHDGL |
| | | YQGLSTATKDTYDALHMQALPPR |
| CD20-C3 | | |
| 1174 | VH | QVQLQQPGAELVKPGASVKMSCKASGYTFTNYNLHWVKQTP |
| | | GQGLEWIGAIYPGNYDTSYNQKFKGKATLTADKSSSTAYMLL |
| | | SSLTSEDSAVYFCARVDFGHSRYWYFDVWGAGTTVTVSS |
| 1175 | VL | QIVLSQSPAILSASPGEKVTMTCRATSSVSSMNWYQQKPGSFPR |
| | | PWIHATSNLASGVPARFSGSGSGTSYSLTISRVEAEDAATYYCQ |
| | | QWTFNPPTFGAGAKLELK |
| CD20-C5 | | |
| 1176 | VH | QVQLQQPGAELVKPGASVKMSCKASGYTFTSYNMHWVKQTP |
| | | GQGLEWIGAIYPGNGDTSYNPKFKGKATLTADKSSRTAYIHLS |
| | | SLTSEDSVVYYCARSYFYGSSSWYFDVWGAGTTVTVSS |
| 1177 | VL | QIILSQSPAILSASPGEKVTLTCRASSSVSSMHWYQQKPGSSPKP |
| | | WIFATSNLASGVPARFTGSGSGTSYSLTISRVEAEDAATYYCQQ |
| | | WIFNPPTFGGGTSLEIK |
| CD20-C2 | | |
| 1178 | VH | QVHLQQSGAELAKPGASVKMSCKASGYTFTNYWMHWVKQR |
| | | PGQGLEWIGFITPTTGYPEYNQKFKDKATLTADKSSSTAYMQL |
| | | SSLTSEDSAVYYCARRKVGKGVYYALDYWGQGTSVTVSS |
| 1179 | VL | DILMTQSPASLSASVGETVTITCRASGNIHNYLAWYQQKQGNS |
| | | PQLLVYNTKTLADGVPSRFSGSGSGTQYSLKINSLQTEDFGTYY |
| | | CQHFWSSPWTFGGGTKLEIK |

CARs that bind to CD22 are known in the art. For example, those disclosed in WO2018/067992 or WO2016/164731. Any known CD22 CAR, for example, the CD22 antigen binding domain of any known CD22 CAR, in the art can be used in accordance with the present disclosure.

Exemplary CD22-binding sequences or CD22 CAR sequences are disclosed in, for example, Tables 6A, 6B, 7A, 7B, 7C, 8A, 8B, 9A, 9B, 10A, and 10B of WO2016164731 and Tables 6-10 of WO2018067992. In some embodiments, the CD22 CAR sequences comprise a CDR, variable region, scFv or full-length sequence of a CD22 CAR disclosed in WO2018067992 or WO2016164731.

In embodiments, the CAR comprises an antigen binding domain that binds to CD22 (CD22 CAR). In some embodiments, the antigen binding domain targets human CD22. In some embodiments, the antigen binding domain includes a single chain Fv sequence as described herein.

The sequences of human CD22 CAR are provided below. In some embodiments, a human CD22 CAR is CAR22-65.

Human CD22 CAR scFv sequence
(SEQ ID NO: 753)
EVQLQQSGPGLVKPSQTLSLTCAISGDSMLSNSDTWNWIRQSPSRGLEWL
GRTYHRSTWYDDYASSVRGRVSINVDTSKNQYSLQLNAVTPEDTGVYYCA
RVRLQDGNSWSDAFDVWGQGTMVTVSSGGGGSGGGGSGGGGSQSALTQPA
SASGSPGQSVTISCTGTSSDVGGYNYVSWYQQHPGKAPKLMIYDVSNRPS
GVSNRFSGSKSGNTASLTISGLQAEDEADYYCSSYTSSSTLYVFGTGTQL
TVL
Human CD22 CAR heavy chain variable region
(SEQ ID NO: 754)
EVQLQQSGPGLVKPSQTLSLTCAISGDSMLSNSDTWNWIRQSPSRGLEWL
GRTYHRSTWYDDYASSVRGRVSINVDTSKNQYSLQLNAVTPEDTGVYYCA
RVRLQDGNSWSDAFDVWGQGTMVTVSS
Human CD22 CAR light chain variable region
(SEQ ID NO 755)
QSALTQPASASGSPGQSVTISCTGTSSDVGGYNYVSWYQQHPGKAPKLMI
YDVSNRPSGVSNRFSGSKSGNTASLTISGLQAEDEADYYCSSYTSSSTLY
VFGTGTQLTVL

In some embodiments, CD22 CARs comprise a sequence, for example, a CDR, VH, VL, scFv, or full-CAR sequence, disclosed in Tables 15-16 and Table 24 below, or a sequence having at least 70%, 75%, 80%, 85%, 90%, 95%, 96%, 97%, 98%, or 99% identity thereto.

TABLE 15
Heavy Chain Variable Domain CDRs of CD22 CAR
(CAR22-65)

| | | SEQ | | SEQ | | SEQ |
|---|---|---|---|---|---|---|
| | | ID | | ID | | ID |
| Candidate | HCDR1 | NO: | HCDR2 | NO: | HCDR3 | NO: |
| CAR22-65 | GDSMLSNS | 744 | RTYHRST | 746 | VRLQDGN | 748 |
| Combined | DTWN | | WYDDYAS | | SWSDAFD | |
| | | | SVRG | | V | |
| CAR22-65 | SNSDTWN | 745 | RTYHRST | 747 | VRLQDGN | 749 |
| Kabat | | | WYDDYAS | | SWSDAFD | |
| | | | SVRG | | V | |
| CD22-65 | GDSMLSNS | 1180 | YHRSTWY | 1181 | VRLQDGN | 1182 |
| Chothia | D | | | | SWSDAFD | |
| | | | | | V | |
| CD22-65 | GDSMLSNS | 1183 | TYHRSTW | 1184 | ARVRLQD | 1185 |
| IMGT | DT | | YD | | GNSWSDA | |
| | | | | | FDV | |

TABLE 16
Light Chain Variable Domain CDRs of CD22 CAR
(CAR22-65). The LC CDR sequences in this table
have the same sequence under the Kabat or
combined definitions.

| | | SEQ | | SEQ | | SEQ |
|---|---|---|---|---|---|---|
| | | ID | | ID | | ID |
| Candidate | LCDR1 | NO: | LCDR2 | NO: | LCDR3 | NO: |
| CAR22-65 | TGTSSDVG | 750 | DVSNRPS | 751 | SSYTSSSTL | 752 |
| Combined | GYNYVS | | | | YV | |
| CD22-65 | TGTSSDVG | 1186 | DVSNRPS | 1189 | SSYTSSSTL | 1192 |
| Kabat | GYNYVS | | | | YV | |
| CD22-65 | TSSDVGGY | 1187 | DVS | 1190 | YTSSSTLY | 1193 |
| Chothia | NY | | | | | |
| CD22-65 | SSDVGGY | 1188 | DVS | 1191 | SSYTSSSTL | 1194 |
| IMGT | NY | | | | YV | |

TABLE 24
Amino acid sequences of exemplary anti-CD22 molecules

| | | |
|---|---|---|
| SEQ ID NO: | Region | Sequence |

CD22-65s

| | | |
|---|---|---|
| 1195 | scFv (VH- | EVQLQQSGPGLVKPSQTLSLTCAISGDSMLSNSDTWNWIRQSP |
| | linker-VL) | SRGLEWLGRTYHRSTWYDDYASSVRGRVSINVDTSKNQYSL |
| | (linker | QLNAVTPEDTGVYYCARVRLQDGNSWSDAFDVWGQGTMVT |
| | shown by | VSSGGGGSQSALTQPASASGSPGQSVTISCTGTSSDVGGYNYV |
| | italics and | SWYQQHPGKAPKLMIYDVSNRPSGVSNRFSGSKSGNTASLTIS |
| | underline) | GLQAEDEADYYCSSYTSSSTLYVFGTGTQLTVL |

CD22-65ss

| | | |
|---|---|---|
| 1196 | scFv (VH - | EVQLQQSGPGLVKPSQTLSLTCAISGDSMLSNSDTWNWIRQSP |
| | VL) | SRGLEWLGRTYHRSTWYDDYASSVRGRVSINVDTSKNQYSL |
| | (no linker | QLNAVTPEDTGVYYCARVRLQDGNSWSDAFDVWGQGTMVT |
| | between | VSSQSALTQPASASGSPGQSVTISCTGTSSDVGGYNYVSWYQ |
| | VH-VL) | QHPGKAPKLMIYDVSNRPSGVSNRFSGSKSGNTASLTISGLQA |
| | | EDEADYYCSSYTSSSTLYVFGTGTQLTVL |

CD22-65sKD

| | | |
|---|---|---|
| 1197 | VH | EVQLQQSGPGLVKPSQTLSLTCAISGDSMLSNSDTWNWIRKSP |
| | | SRGLEWLGRTYHRSTWYDDYASSVRGRVSINVDTSKNQYSL |
| | | QLNAVTPEDTGVYYCARVRLQDGNSWSDAFDVWGQGTMVT |
| | | VSS |
| 1198 | VL | QSALTQPASASGSPGQSVTISCTGTSSDVGGYNYVSWYQDHP |
| | | GKAPKLMIYDVSNRPSGVSNRFSGSKSGNTASLTISGLQAEDE |
| | | ADYYCSSYTSSSTLYVFGTGTQLTVL |
| 1199 | scFv (VH- | EVQLQQSGPGLVKPSQTLSLTCAISGDSMLSNSDTWNWIRKSP |
| | linker-VL) | SRGLEWLGRTYHRSTWYDDYASSVRGRVSINVDTSKNQYSL |
| | | QLNAVTPEDTGVYYCARVRLQDGNSWSDAFDVWGQGTMVT |
| | | VSSGGGGSQSALTQPASASGSPGQSVTISCTGTSSDVGGYNYV |
| | | SWYQDHPGKAPKLMIYDVSNRPSGVSNRFSGSKSGNTASLTIS |
| | | GLQAEDEADYYCSSYTSSSTLYVFGTGTQLTVL |

CD22-64

| | | |
|---|---|---|
| 1200 | HC CDR1 | SNSDTWN |
| | (Kabat) | |
| 1201 | HC CDR2 | RTYHRSTWYDDYASSVRG |
| | (Kabat) | |
| 1202 | HC CDR3 | VRLQDGNSWSDAFDV |
| | (Kabat) | |
| 1203 | HC CDR1 | GDSVLSNSD |
| | (Chothia) | |
| 1204 | HC CDR2 | YHRSTWY |
| | (Chothia) | |
| 1205 | HC CDR3 | VRLQDGNSWSDAFDV |
| | (Chothia) | |
| 1206 | HC CDR1 | GDSVLSNSDT |
| | (IMGT) | |
| 1207 | HC CDR2 | TYHRSTWYD |
| | (IMGT) | |
| 1208 | HC CDR3 | ARVRLQDGNSWSDAFDV |
| | (IMGT) | |
| 1209 | HC CDR1 | GDSVLSNSDTWN |
| | (Combined) | |
| 1210 | HC CDR2 | RTYHRSTWYDDYASSVRG |
| | (Combined) | |
| 1211 | HC CDR3 | VRLQDGNSWSDAFDV |
| | (Combined) | |
| 1212 | VH | EVQLQQSGPGLVKPSQTLPLTCAISGDSVLSNSDTWNWIRQSP |
| | | SRGLEWLGRTYHRSTWYDDYASSVRGRVSINVDTSKNQYSL |
| | | QLNAVTPEDTGVYYCARVRLQDGNSWSDAFDVWGQGTMVT |
| | | VSS |
| 1213 | LC CDR1 | TGTSSDVGGYNYVS |
| | (Kabat) | |
| 1214 | LC CDR2 | DVSNRPS |
| | (Kabat) | |
| 1215 | LC CDR3 | SSYTSSSTLYV |
| | (Kabat) | |
| 1216 | LC CDR1 | TSSDVGGYNY |
| | (Chothia) | |
| 1217 | LC CDR2 | DVS |
| | (Chothia) | |
| 1218 | LC CDR3 | YTSSSTLY |
| | (Chothia) | |
| 1219 | LC CDR1 | SSDVGGYNY |
| | (IMGT) | |
| 1220 | LC CDR2 | DVS |
| | (IMGT) | |
| 1221 | LC CDR3 | SSYTSSSTLYV |
| | (IMGT) | |
| 1222 | LC CDR1 | TGTSSDVGGYNYVS |
| | (Combined) | |
| 1223 | LC CDR2 | DVSNRPS |
| | (Combined) | |
| 1224 | LC CDR3 | SSYTSSSTLYV |
| | (Combined) | |
| 1225 | VL | QSALTQPASASGSPGQSVTISCTGTSSDVGGYNYVSWYQQHP |
| | | GKAPKLMIYDVSNRPSGVSNRFSGSKSGNTASLTISGLQAEDE |
| | | ADYYCSSYTSSSTLYVFGTGTQLTVL |
| 1226 | scFv (VH- | EVQLQQSGPGLVKPSQTLPLTCAISGDSVLSNSDTWNWIRQSP |
| | linker-VL) | SRGLEWLGRTYHRSTWYDDYASSVRGRVSINVDTSKNQYSL |
| | | QLNAVTPEDTGVYYCARVRLQDGNSWSDAFDVWGQGTMVT |
| | | VSSGGGGSGGGGSGGGGPQSALTQPASASGSPGQSVTISCTGT |
| | | SSDVGGYNYVSWYQQHPGKAPKLMIYDVSNRPSGVSNRFSG |
| | | SKSGNTASLTISGLQAEDEADYYCSSYTSSSTLYVFGTGTQLT |
| | | VL |

CD22-57

| | | |
|---|---|---|
| 1227 | HC CDR1 | NNNAAWN |
| | (Kabat) | |
| 1228 | HC CDR2 | RTYHRSTWYNDYVGSVKS |
| | (Kabat) | |
| 1229 | HC CDR3 | ETDYGDYGAFDI |
| | (Kabat) | |
| 1230 | HC CDR1 | GDSVSNNNA |
| | (Chothia) | |
| 1231 | HC CDR2 | YHRSTWY |
| | (Chothia) | |
| 1232 | HC CDR3 | ETDYGDYGAFDI |
| | (Chothia) | |
| 1233 | HC CDR1 | GDSVSNNNAA |
| | (IMGT) | |
| 1234 | HC CDR2 | TYHRSTWYN |
| | (IMGT) | |
| 1235 | HC CDR3 | ARETDYGDYGAFDI |
| | (IMGT) | |
| 1236 | HC CDR1 | GDSVSNNNAAWN |
| | (Combined) | |
| 1237 | HC CDR2 | RTYHRSTWYNDYVGSVKS |
| | (Combined) | |
| 1238 | HC CDR3 | ETDYGDYGAFDI |
| | (Combined) | |
| 1239 | VH | EVQLQQSGPGLVKPSQTLSLTCAISGDSVSNNNAAWNWIRQS |
| | | PSRGLEWLGRTYHRSTWYNDYVGSVKSRITINPDTSKNQFSL |
| | | QLNSVTPEDTAVYYCARETDYGDYGAFDIWGQGTTVTVSS |
| 1240 | LC CDR1 | TGSRNDIGAYESVS |
| | (Kabat) | |
| 1241 | LC CDR2 | GVNNRPS |
| | (Kabat) | |
| 1242 | LC CDR3 | SSHTTTSTLYV |
| | (Kabat) | |
| 1243 | LC CDR1 | SRNDIGAYES |
| | (Chothia) | |
| 1244 | LC CDR2 | GVN |
| | (Chothia) | |
| 1245 | LC CDR3 | HTTTSTLY |
| | (Chothia) | |
| 1246 | LC CDR1 | RNDIGAYES |
| | (IMGT) | |
| 1247 | LC CDR2 | GVN |
| | (IMGT) | |
| 1248 | LC CDR3 | SSHTTTSTLYV |
| | (IMGT) | |
| 1249 | LC CDR1 | TGSRNDIGAYESVS |
| | (Combined) | |
| 1250 | LC CDR2 | GVNNRPS |
| | (Combined) | |
| 1251 | LC CDR3 | SSHTTTSTLYV |
| | (Combined) | |
| 1252 | VL | QSALTQPASVSGSPGQSITISCTGSRNDIGAYESVSWYQQHPG |
| | | NAPKLIIHGVNNRPSGVFDRFSVSQSGNTASLTISGLQAEDEAD |
| | | YYCSSHTTTSTLYVFGTGTKVTVL |
| 1253 | scFv (VH- | EVQLQQSGPGLVKPSQTLSLTCAISGDSVSNNNAAWNWIRQS |
| | linker-VL) | PSRGLEWLGRTYHRSTWYNDYVGSVKSRITINPDTSKNQFSL |
| | | QLNSVTPEDTAVYYCARETDYGDYGAFDIWGQGTTVTVSSG |
| | | GGGSGGGGSGGGGSQSALTQPASVSGSPGQSITISCTGSRNDIG |
| | | AYESVSWYQQHPGNAPKLIIHGVNNRPSGVFDRFSVSQSGNT |
| | | ASLTISGLQAEDEADYYCSSHTTTSTLYVFGTGTKVTVL |

CD22-58

| | | |
|---|---|---|
| 1254 | HC CDR1 | SNSAAWN |
| | (Kabat) | |
| 1255 | HC CDR2 | RTFYRSKWYNDYAVSVKG |
| | (Kabat) | |
| 1256 | HC CDR3 | GDYYYGLDV |
| | (Kabat) | |
| 1257 | HC CDR1 | GDSVSSNSA |
| | (Chothia) | |
| 1258 | HC CDR2 | FYRSKWY |
| | (Chothia) | |
| 1259 | HC CDR3 | GDYYYGLDV |
| | (Chothia) | |
| 1260 | HC CDR1 | GDSVSSNSAA |
| | (IMGT) | |
| 1261 | HC CDR2 | TFYRSKWYN |
| | (IMGT) | |
| 1262 | HC CDR3 | AGGDYYYGLDV |
| | (IMGT) | |
| 1263 | HC CDR1 | GDSVSSNSAAWN |
| | (Combined) | |
| 1264 | HC CDR2 | RTFYRSKWYNDYAVSVKG |
| | (Combined) | |
| 1265 | HC CDR3 | GDYYYGLDV |
| | (Combined) | |
| 1266 | VH | EVQLQQSGPGLVNPSQTLSITCAISGDSVSSNSAAWNWIRQSP |
| | | SRGLEWLGRTFYRSKWYNDYAVSVKGRITISPDTSKNQFSLQ |
| | | LNSVTPEDTAVYYCAGGDYYYGLDVWGQGTTVTVSS |
| 1267 | LC CDR1 | TGSSSDVGGYNSVS |
| | (Kabat) | |
| 1268 | LC CDR2 | EVINRPS |
| | (Kabat) | |
| 1269 | LC CDR3 | SSYTSSSTYV |
| | (Kabat) | |
| 1270 | LC CDR1 | SSSDVGGYNS |
| | (Chothia) | |
| 1271 | LC CDR2 | EVI |
| | (Chothia) | |
| 1272 | LC CDR3 | YTSSSTY |
| | (Chothia) | |
| 1273 | LC CDR1 | SSDVGGYNS |
| | (IMGT) | |
| 1274 | LC CDR2 | EVI |
| | (IMGT) | |
| 1275 | LC CDR3 | SSYTSSSTYV |
| | (IMGT) | |
| 1276 | LC CDR1 | TGSSSDVGGYNSVS |
| | (Combined) | |
| 1277 | LC CDR2 | EVINRPS |
| | (Combined) | |
| 1278 | LC CDR3 | SSYTSSSTYV |
| | (Combined) | |
| 1279 | VL | QSALTQPASVSGSPGQSITISCTGSSSDVGGYNSVSWYQQHPG |
| | | KAPKLMIYEVINRPSGVSHRFSGSKSGNTASLTISGLQAEDEA |
| | | DYYCSSYTSSSTYVFGTGTKVTVL |
| 1280 | scFv (VH- | EVQLQQSGPGLVNPSQTLSITCAISGDSVSSNSAAWNWIRQSP |
| | linker-VL) | SRGLEWLGRTFYRSKWYNDYAVSVKGRITISPDTSKNQFSLQ |
| | | LNSVTPEDTAVYYCAGGDYYYGLDVWGQGTTVTVSSGGGGS |
| | | GGGGSGGGGSQSALTQPASVSGSPGQSITISCTGSSSDVGGYN |
| | | SVSWYQQHPGKAPKLMIYEVINRPSGVSHRFSGSKSGNTASLT |
| | | ISGLQAEDEADYYCSSYTSSSTYVFGTGTKVTVL |

CD22-59

| | | |
|---|---|---|
| 1281 | HC CDR1 | SNSDTWN |
| | (Kabat) | |
| 1282 | HC CDR2 | RTYHRSTWYDDYASSVRG |
| | (Kabat) | |
| 1283 | HC CDR3 | DRLQDGNSWSDAFDV |
| | (Kabat) | |
| 1284 | HC CDR1 | GDSVLSNSD |
| | (Chothia) | |
| 1285 | HC CDR2 | YHRSTWY |
| | (Chothia) | |
| 1286 | HC CDR3 | DRLQDGNSWSDAFDV |
| | (Chothia) | |
| 1287 | HC CDR1 | GDSVLSNSDT |
| | (IMGT) | |
| 1288 | HC CDR2 | TYHRSTWYD |
| | (IMGT) | |
| 1289 | HC CDR3 | ARDRLQDGNSWSDAFDV |
| | (IMGT) | |
| 1290 | HC CDR1 | GDSVLSNSDTWN |
| | (Combined) | |
| 1291 | HC CDR2 | RTYHRSTWYDDYASSVRG |
| | (Combined) | |
| 1292 | HC CDR3 | DRLQDGNSWSDAFDV |
| | (Combined) | |
| 1293 | VH | EVQLQQSGPGLVKPSQTLSLTCAISGDSVLSNSDTWNWIRQSP |
| | | SRGLEWLGRTYHRSTWYDDYASSVRGRVSINVDTSKNQYSL |
| | | QLNAVTPEDTGVYYCARDRLQDGNSWSDAFDVWGQGTMVT |
| | | VSS |
| 1294 | LC CDR1 | TGSSSDIGGFNYVS |
| | (Kabat) | |
| 1295 | LC CDR2 | EVTNRPS |
| | (Kabat) | |
| 1296 | LC CDR3 | SSYASGSPLYV |
| | (Kabat) | |
| 1297 | LC CDR1 | SSSDIGGFNY |
| | (Chothia) | |
| 1298 | LC CDR2 | EVT |
| | (Chothia) | |
| 1299 | LC CDR3 | YASGSPLY |
| | (Chothia) | |
| 1300 | LC CDR1 | SSDIGGFNY |
| | (IMGT) | |
| 1301 | LC CDR2 | EVT |
| | (IMGT) | |
| 1302 | LC CDR3 | SSYASGSPLYV |
| | (IMGT) | |
| 1303 | LC CDR1 | TGSSSDIGGFNYVS |
| | (Combined) | |
| 1304 | LC CDR2 | EVTNRPS |
| | (Combined) | |
| 1305 | LC CDR3 | SSYASGSPLYV |
| | (Combined) | |
| 1306 | VL | QSALTQPASVSGSPGQSITISCTGSSSDIGGFNYVSWYQQHAGE |
| | | APKLMIYEVTNRPSGVSDRFSGSKSDNTASLTISGLQAEDEAD |
| | | YYCSSYASGSPLYVFGTGTKVTVL |
| 1307 | scFv (VH- | EVQLQQSGPGLVKPSQTLSLTCAISGDSVLSNSDTWNWIRQSP |
| | linker-VL) | SRGLEWLGRTYHRSTWYDDYASSVRGRVSINVDTSKNQYSL |
| | | QLNAVTPEDTGVYYCARDRLQDGNSWSDAFDVWGQGTMVT |
| | | VSSGGGGSGGGGSGGGGSQSALTQPASVSGSPGQSITISCTGSS |
| | | SDIGGFNYVSWYQQHAGEAPKLMIYEVTNRPSGVSDRFSGSK |
| | | SDNTASLTISGLQAEDEADYYCSSYASGSPLYVFGTGTKVTVL |

CD22-60

| | | |
|---|---|---|
| 1308 | HC CDR1 | SNSDTWN |
| | (Kabat) | |
| 1309 | HC CDR2 | RTYHRSTWYDDYASSVRG |
| | (Kabat) | |
| 1310 | HC CDR3 | DRLQDGNSWSDAFDV |
| | (Kabat) | |
| 1311 | HC CDR1 | GDSVLSNSD |
| | (Chothia) | |
| 1312 | HC CDR2 | YHRSTWY |
| | (Chothia) | |
| 1313 | HC CDR3 | DRLQDGNSWSDAFDV |
| | (Chothia) | |
| 1314 | HC CDR1 | GDSVLSNSDT |
| | (IMGT) | |
| 1315 | HC CDR2 | TYHRSTWYD |
| | (IMGT) | |
| 1316 | HC CDR3 | ARDRLQDGNSWSDAFDV |
| | (IMGT) | |
| 1317 | HC CDR1 | GDSVLSNSDTWN |
| | (Combined) | |
| 1318 | HC CDR2 | RTYHRSTWYDDYASSVRG |
| | (Combined) | |
| 1319 | HC CDR3 | DRLQDGNSWSDAFDV |
| | (Combined) | |
| 1320 | VH | EVQLQQSGPGLVKPSQTLSLTCAISGDSVLSNSDTWNWIRQSP |
| | | SRGLEWLGRTYHRSTWYDDYASSVRGRVSINVDTSKNQYSL |
| | | QLNAVTPEDTGVYYCARDRLQDGNSWSDAFDVWGQGTMVT |
| | | VSS |
| 1321 | LC CDR1 | TGTSSDIGGYNYVS |
| | (Kabat) | |
| 1322 | LC CDR2 | EVSNRPS |
| | (Kabat) | |
| 1323 | LC CDR3 | SSYTSSSTLYV |
| | (Kabat) | |
| 1324 | LC CDR1 | TSSDIGGYNY |
| | (Chothia) | |
| 1325 | LC CDR2 | EVS |
| | (Chothia) | |
| 1326 | LC CDR3 | YTSSSTLY |
| | (Chothia) | |
| 1327 | LC CDR1 | SSDIGGYNY |
| | (IMGT) | |
| 1328 | LC CDR2 | EVS |
| | (IMGT) | |
| 1329 | LC CDR3 | SSYTSSSTLYV |
| | (IMGT) | |
| 1330 | LC CDR1 | TGTSSDIGGYNYVS |
| | (Combined) | |
| 1331 | LC CDR2 | EVSNRPS |
| | (Combined) | |
| 1332 | LC CDR3 | SSYTSSSTLYV |
| | (Combined) | |
| 1333 | VL | QSALTQPASVSGSPGQSITFSCTGTSSDIGGYNYVSWYQQHPG |
| | | KAPKLMIYEVSNRPSGVSNRFSGTKSGNTASLTISGLQAEDEA |
| | | DYYCSSYTSSSTLYVFGTGTKLTVL |
| 1334 | scFv (VH- | EVQLQQSGPGLVKPSQTLSLTCAISGDSVLSNSDTWNWIRQSP |
| | linker-VL) | SRGLEWLGRTYHRSTWYDDYASSVRGRVSINVDTSKNQYSL |
| | | QLNAVTPEDTGVYYCARDRLQDGNSWSDAFDVWGQGTMVT |
| | | VSSGGGGSGGGGSGSGGSQSALTQPASVSGSPGQSITFSCTGT |
| | | SSDIGGYNYVSWYQQHPGKAPKLMIYEVSNRPSGVSNRFSGT |
| | | KSGNTASLTISGLQAEDEADYYCSSYTSSSTLYVFGTGTKLTV |
| | | L |

CD22-61

| | | |
|---|---|---|
| 1335 | HC CDR1 | SNSDTWN |
| | (Kabat) | |
| 1336 | HC CDR2 | RTYHRSTWYDDYASSVRG |
| | (Kabat) | |
| 1337 | HC CDR3 | DRLQDGNSWSDAFDV |
| | (Kabat) | |
| 1338 | HC CDR1 | GDSVLSNSD |
| | (Chothia) | |
| 1339 | HC CDR2 | YHRSTWY |
| | (Chothia) | |
| 1340 | HC CDR3 | DRLQDGNSWSDAFDV |
| | (Chothia) | |
| 1341 | HC CDR1 | GDSVLSNSDT |
| | (IMGT) | |
| 1342 | HC CDR2 | TYHRSTWYD |
| | (IMGT) | |
| 1343 | HC CDR3 | ARDRLQDGNSWSDAFDV |
| | (IMGT) | |
| 1344 | HC CDR1 | GDSVLSNSDTWN |
| | (Combined) | |
| 1345 | HC CDR2 | RTYHRSTWYDDYASSVRG |
| | (Combined) | |
| 1346 | HC CDR3 | DRLQDGNSWSDAFDV |
| | (Combined) | |
| 1347 | VH | QVQLQESGPGLVKPSQTLSLTCAISGDSVLSNSDTWNWIRQSP |
| | | SRGLEWLGRTYHRSTWYDDYASSVRGRVSINVDTSKNQYSL |
| | | QLNAVTPEDTGVYYCARDRLQDGNSWSDAFDVWGQGTMVT |
| | | VSS |
| 1348 | LC CDR1 | TGTSSDVGGYNYVS |
| | (Kabat) | |
| 1349 | LC CDR2 | EVSNRPS |
| | (Kabat) | |
| 1350 | LC CDR3 | SSYTSSSTLYV |
| | (Kabat) | |
| 1351 | LC CDR1 | TSSDVGGYNY |
| | (Chothia) | |
| 1352 | LC CDR2 | EVS |
| | (Chothia) | |
| 1353 | LC CDR3 | YTSSSTLY |
| | (Chothia) | |
| 1354 | LC CDR1 | SSDVGGYNY |
| | (IMGT) | |
| 1355 | LC CDR2 | EVS |
| | (IMGT) | |
| 1356 | LC CDR3 | SSYTSSSTLYV |
| | (IMGT) | |
| 1357 | LC CDR1 | TGTSSDVGGYNYVS |
| | (Combined) | |
| 1358 | LC CDR2 | EVSNRPS |
| | (Combined) | |
| 1359 | LC CDR3 | SSYTSSSTLYV |
| | (Combined) | |
| 1360 | VL | QSALTQPASVSGSPGQSITISCTGTSSDVGGYNYVSWYQQHPG |
| | | KAPKLMIYEVSNRPSGVSNRFSGSKSGNTASLTISGLQAEDEA |
| | | DYYCSSYTSSSTLYVFGTGTKVTVL |
| 1361 | scFv (VH- | QVQLQESGPGLVKPSQTLSLTCAISGDSVLSNSDTWNWIRQSP |
| | linker-VL) | SRGLEWLGRTYHRSTWYDDYASSVRGRVSINVDTSKNQYSL |
| | | QLNAVTPEDTGVYYCARDRLQDGNSWSDAFDVWGQGTMVT |
| | | VSSGGGGSGGGGSGSGGSQSALTQPASVSGSPGQSITISCTGTS |
| | | SDVGGYNYVSWYQQHPGKAPKLMIYEVSNRPSGVSNRFSGS |
| | | KSGNTASLTISGLQAEDEADYYCSSYTSSSTLYVFGTGTKVTV |
| | | L |

CD22-62

| | | |
|---|---|---|
| 1362 | HC CDR1 | SNSDTWN |
| | (Kabat) | |
| 1363 | HC CDR2 | RTYHRSTWYDDYASSVRG |
| | (Kabat) | |
| 1364 | HC CDR3 | DRLQDGNSWSDAFDV |
| | (Kabat) | |
| 1365 | HC CDR1 | GDSVLSNSD |
| | (Chothia) | |
| 1366 | HC CDR2 | YHRSTWY |
| | (Chothia) | |
| 1367 | HC CDR3 | DRLQDGNSWSDAFDV |
| | (Chothia) | |
| 1368 | HC CDR1 | GDSVLSNSDT |
| | (IMGT) | |
| 1369 | HC CDR2 | TYHRSTWYD |
| | (IMGT) | |
| 1370 | HC CDR3 | ARDRLQDGNSWSDAFDV |
| | (IMGT) | |
| 1371 | HC CDR1 | GDSVLSNSDTWN |
| | (Combined) | |
| 1372 | HC CDR2 | RTYHRSTWYDDYASSVRG |
| | (Combined) | |
| 1373 | HC CDR3 | DRLQDGNSWSDAFDV |
| | (Combined) | |
| 1374 | VH | EVQLQQSGPGLVKPSQTLSLTCAISGDSVLSNSDTWNWIRQSP |
| | | SRGLEWLGRTYHRSTWYDDYASSVRGRVSINVDTSKNQYSL |
| | | QLNAVTPEDTGVYYCARDRLQDGNSWSDAFDVWGQGTMVT |
| | | VSS |
| 1375 | LC CDR1 | TGTSSDVGGYNYVS |
| | (Kabat) | |
| 1376 | LC CDR2 | DVSNRPS |
| | (Kabat) | |
| 1377 | LC CDR3 | SSYTSSSTLYV |
| | (Kabat) | |
| 1378 | LC CDR1 | TSSDVGGYNY |
| | (Chothia) | |
| 1379 | LC CDR2 | DVS |
| | (Chothia) | |
| 1380 | LC CDR3 | YTSSSTLY |
| | (Chothia) | |
| 1381 | LC CDR1 | SSDVGGYNY |
| | (IMGT) | |
| 1382 | LC CDR2 | DVS |
| | (IMGT) | |
| 1383 | LC CDR3 | SSYTSSSTLYV |
| | (IMGT) | |
| 1384 | LC CDR1 | TGTSSDVGGYNYVS |
| | (Combined) | |
| 1385 | LC CDR2 | DVSNRPS |
| | (Combined) | |
| 1386 | LC CDR3 | SSYTSSSTLYV |
| | (Combined) | |
| 1387 | VL | QSALTQPASVSGSPGQSITISCTGTSSDVGGYNYVSWYQQHPG |
| | | KAPKLMIYDVSNRPSGVSNRFSGSKSGNTASLTISGLQAEDEA |
| | | DYYCSSYTSSSTLYVFGTGTKVTVL |
| 1388 | scFv (VH- | EVQLQQSGPGLVKPSQTLSLTCAISGDSVLSNSDTWNWIRQSP |
| | linker-VL) | SRGLEWLGRTYHRSTWYDDYASSVRGRVSINVDTSKNQYSL |
| | | QLNAVTPEDTGVYYCARDRLQDGNSWSDAFDVWGQGTMVT |
| | | VSSGGGGSGGGGSGGGGSQSALTQPASVSGSPGQSITISCTGTS |
| | | SDVGGYNYVSWYQQHPGKAPKLMIYDVSNRPSGVSNRFSGS |
| | | KSGNTASLTISGLQAEDEADYYCSSYTSSSTLYVFGTGTKVTV |
| | | L |

CD22-63

| | | |
|---|---|---|
| 1389 | HC CDR1 | SNSDTWN |
| | (Kabat) | |
| 1390 | HC CDR2 | RTYHRSTWYDDYASSVRG |
| | (Kabat) | |
| 1391 | HC CDR3 | DRLQDGNSWSDAFDV |
| | (Kabat) | |
| 1392 | HC CDR1 | GDSVLSNSD |
| | (Chothia) | |
| 1393 | HC CDR2 | YHRSTWY |
| | (Chothia) | |
| 1394 | HC CDR3 | DRLQDGNSWSDAFDV |
| | (Chothia) | |
| 1395 | HC CDR1 | GDSVLSNSDT |
| | (IMGT) | |
| 1396 | HC CDR2 | TYHRSTWYD |
| | (IMGT) | |
| 1397 | HC CDR3 | ARDRLQDGNSWSDAFDV |
| | (IMGT) | |
| 1398 | HC CDR1 | GDSVLSNSDTWN |
| | (Combined) | |
| 1399 | HC CDR2 | RTYHRSTWYDDYASSVRG |
| | (Combined) | |
| 1400 | HC CDR3 | DRLQDGNSWSDAFDV |
| | (Combined) | |
| 1401 | VH | EVQLQQSGPGLVKPSQTLSLTCAISGDSVLSNSDTWNWIRQSP |
| | | SRGLEWLGRTYHRSTWYDDYASSVRGRVSINVDTSKNQYSL |
| | | QLNAVTPEDTGVYYCARDRLQDGNSWSDAFDVWGQGTMVT |
| | | VSS |
| 1402 | LC CDR1 | TGTSSDVGGYNYVS |
| | (Kabat) | |
| 1403 | LC CDR2 | EVSNRPS |
| | (Kabat) | |
| 1404 | LC CDR3 | SSYTSSSTLYI |
| | (Kabat) | |
| 1405 | LC CDR1 | TSSDVGGYNY |
| | (Chothia) | |
| 1406 | LC CDR2 | EVS |
| | (Chothia) | |
| 1407 | LC CDR3 | YTSSSTLY |
| | (Chothia) | |
| 1408 | LC CDR1 | SSDVGGYNY |
| | (IMGT) | |
| 1409 | LC CDR2 | EVS |
| | (IMGT) | |
| 1410 | LC CDR3 | SSYTSSSTLYI |
| | (IMGT) | |
| 1411 | LC CDR1 | TGTSSDVGGYNYVS |
| | (Combined) | |
| 1412 | LC CDR2 | EVSNRPS |
| | (Combined) | |
| 1413 | LC CDR3 | SSYTSSSTLYI |
| | (Combined) | |
| 1414 | VL | QSALTQPASVSGSPGQSITISCTGTSSDVGGYNYVSWYQQHPG |
| | | KAPKLMIYEVSNRPSGVSNRFSGSKSGNTASLTISGLQAEDEA |
| | | DYYCSSYTSSSTLYIFGTGTKVTVL |
| 1415 | scFv (VH- | EVQLQQSGPGLVKPSQTLSLTCAISGDSVLSNSDTWNWIRQSP |
| | linker-VL) | SRGLEWLGRTYHRSTWYDDYASSVRGRVSINVDTSKNQYSL |
| | | QLNAVTPEDTGVYYCARDRLQDGNSWSDAFDVWGQGTMVT |
| | | VSSGGGGSGGGGSGGGGSQSALTQPASVSGSPGQSITISCTGTS |
| | | SDVGGYNYVSWYQQHPGKAPKLMIYEVSNRPSGVSNRFSGS |
| | | KSGNTASLTISGLQAEDEADYYCSSYTSSSTLYIFGTGTKVTVL |

CARs that bind to EGFR are known in the art. For example, those disclosed in WO2014/130657, incorporated by reference herein. Any known EGFR CAR, for example, the EGFR antigen binding domain of any known EGFR CAR, in the art can be used in accordance with the present disclosure. Exemplary EGFRvIII CARs can include a CDR, a variable region, an scFv, or a full-length CAR sequence disclosed in WO2014/130657, for example, Table 2 of WO2014/130657, incorporated herein by reference.

CARs that bind to CD123 are known in the art. For example, those disclosed in WO2014/130635 or WO2016/028896. Any known CD123 CAR, for example, the CD123 antigen binding domain of any known CD123 CAR, in the art can be used in accordance with the present disclosure. For example, CAR1 to CAR8 disclosed in WO2014/130635; or CAR123-1 to CAR123-4 and hzCAR123-1 to hzCAR123-32, disclosed in WO2016/028896. The amino acid and nucleotide sequences encoding the CD123 CAR molecules and antigen binding domains (for example, including one, two, three VH CDRs; and one, two, three VL CDRs according to Kabat or Chothia), are specified in WO 2014/130635 and WO2016/028896. CARs that bind to CLL-1 are known in the art. For example, those disclosed in US2016/0051651A1, incorporated herein by reference. Any known CLL-1 CAR, for example, the CLL-1 antigen binding domain of any known CLL-1 CAR, in the art can be used in accordance with the present disclosure.

In some embodiments, the CAR comprises a CLL-1 CAR or antigen binding domain according to Table 2 of WO2016/014535, incorporated herein by reference. The amino acid and nucleotide sequences encoding the CLL-1 CAR molecules and antigen binding domains (for example, including one, two, three VH CDRs; and one, two, three VL CDRs according to Kabat or Chothia), are specified in WO2016/014535.

CARs that bind to CD33 are known in the art. For example, those disclosed in US2016/0096892A1 and WO2016/014576, incorporated by reference herein. Any known CD33 CAR, for example, the CD33 antigen binding domain of any known CD33 CAR, in the art can be used in accordance with the present disclosure. For example, CAR33-1 to CAR33-9 disclosed in WO2016/014576.

In some embodiments, the CAR comprises a CD33 CAR or antigen binding domain according to Table 2 or 9 of WO2016/014576, incorporated herein by reference. The amino acid and nucleotide sequences encoding the CD33 CAR molecules and antigen binding domains (for example, including one, two, three VH CDRs; and one, two, three VL CDRs according to Kabat or Chothia), are specified in WO2016/014576.

CARs that bind to mesothelin are known in the art. For example, those disclosed in WO2015090230 and WO2017112741, for example, Tables 2, 3, 4, and 5 of WO2017112741, incorporated herein by reference, that bind human mesothelin. Any known mesothelin CAR, for example, the mesothelin antigen binding domain of any known mesothelin CAR, in the art can be used in accordance with the present disclosure.

CARs that bind to GFR ALPHA-4 are known in the art. For example, those disclosed in WO2016/025880. Any known GFR ALPHA-4 CAR, for example, the GFR ALPHA-4 antigen binding domain of any known GFR ALPHA-4 CAR, in the art can be used in accordance with the present disclosure. The amino acid and nucleotide sequences encoding the GFR ALPHA-4 CAR molecules and antigen binding domains (for example, including one, two, three VH CDRs; and one, two, three VL CDRs according to Kabat or Chothia), are specified in WO2016/025880.

Antigen Binding Domain Structures

In some embodiments, the antigen binding domain of the encoded CAR molecule comprises an antibody, an antibody fragment, an scFv, a Fv, a Fab, a (Fab′)2, a single domain antibody (SDAB), a VH or VL domain, a camelid VHH domain or a bi-functional (e.g. bi-specific) hybrid antibody (e.g., Lanzavecchia et al., Eur. J. Immunol. 17, 105 (1987)). In some instances, scFvs can be prepared according to method known in the art (see, for example, Bird et al., (1988) Science 242:423-426 and Huston et al., (1988) Proc. Natl. Acad. Sci. USA 85:5879-5883). ScFv molecules can be produced by linking VH and VL regions together using flexible polypeptide linkers. The scFv molecules comprise a linker (e.g., a Ser-Gly linker) with an optimized length and/or amino acid composition. The linker length can greatly affect how the variable regions of a scFv fold and interact. In fact, if a short polypeptide linker is employed (e.g., between 5-10 amino acids) intrachain folding is prevented. Interchain folding is also required to bring the two variable regions together to form a functional epitope binding site. For examples of linker orientation and size see, e.g., Hollinger et al. 1993 Proc Natl Acad. Sci. U.S.A. 90:6444-6448, U.S. Patent Application Publication Nos. 2005/0100543, 2005/0175606, 2007/0014794, and PCT publication Nos. WO2006/020258 and WO2007/024715, is incorporated herein by reference.

An scFv can comprise a linker of at least 1, 2, 3, 4, 5, 6, 7, 8, 9, 10, 11, 12, 13, 14, 15, 16, 17, 18, 19, 20, 25, 30, 35, 40, 45, 50, or more amino acid residues between its VL and VH regions. The linker sequence may comprise any naturally occurring amino acid. In some embodiments, the linker sequence comprises amino acids glycine and serine. In another embodiment, the linker sequence comprises sets of glycine and serine repeats such as (Gly4Ser)n, where n is a positive integer equal to or greater than 1 (SEQ ID NO:22). In some embodiments, the linker can be (Gly4Ser)4 (SEQ ID NO:29) or (Gly4Ser)3(SEQ ID NO:30). Variation in the linker length may retain or enhance activity, giving rise to superior efficacy in activity studies.

In another aspect, the antigen binding domain is a T cell receptor (“TCR”), or a fragment thereof, for example, a single chain TCR (scTCR). Methods to make such TCRs are known in the art. See, e.g., Willemsen R A et al, Gene Therapy 7: 1369-1377 (2000); Zhang T et al, Cancer Gene Ther 11: 487-496 (2004); Aggen et al, Gene Ther. 19(4):365-74 (2012) (references are incorporated herein by its entirety). For example, scTCR can be engineered that contains the Vα and Vβ genes from a T cell clone linked by a linker (e.g., a flexible peptide). This approach is very useful to cancer associated target that itself is intracellular, however, a fragment of such antigen (peptide) is presented on the surface of the cancer cells by MHC.

In certain embodiments, the encoded antigen binding domain has a binding affinity KD of 10⁻⁴ M to 10⁻⁸ M.

In some embodiments, the encoded CAR molecule comprises an antigen binding domain that has a binding affinity KD of 10⁻⁴ M to 10⁻⁸ M, e.g., 10⁻⁵ M to 10⁻⁷ M, e.g., 10⁻⁶ M or 10⁻⁷ M, for the target antigen. In some embodiments, the antigen binding domain has a binding affinity that is at least five-fold, 10-fold, 20-fold, 30-fold, 50-fold, 100-fold or 1,000-fold less than a reference antibody, e.g., an antibody described herein. In some embodiments, the encoded antigen binding domain has a binding affinity at least 5-fold less than a reference antibody (e.g., an antibody from which the antigen binding domain is derived). In some aspects such antibody fragments are functional in that they provide a biological response that can include, but is not limited to, activation of an immune response, inhibition of signal-transduction origination from its target antigen, inhibition of kinase activity, and the like, as will be understood by a skilled artisan.

In some aspects, the antigen binding domain of the CAR is a scFv antibody fragment that is humanized compared to the murine sequence of the scFv from which it is derived.

In some aspects, the antigen binding domain of a CAR described herein (e.g., a scFv) is encoded by a nucleic acid molecule whose sequence has been codon optimized for expression in a mammalian cell. In some aspects, entire CAR construct of the invention is encoded by a nucleic acid molecule whose entire sequence has been codon optimized for expression in a mammalian cell. Codon optimization refers to the discovery that the frequency of occurrence of synonymous codons (i.e., codons that code for the same amino acid) in coding DNA is biased in different species. Such codon degeneracy allows an identical polypeptide to be encoded by a variety of nucleotide sequences. A variety of codon optimization methods is known in the art, and include, e.g., methods disclosed in at least U.S. Pat. Nos. 5,786,464 and 6,114,148.

Specific antigen antibody pairs are known in the art. Non-limiting exemplary embodiments of antigen antibody pairs and components thereof are provided herein above in the section titled Targets and below.

CD19

In some embodiments, the antigen binding domain binds to CD19 and has the same or a similar binding specificity as the FMC63 scFv fragment described in Nicholson et al. Mol. Immun. 34 (16-17): 1157-1165 (1997). In some embodiments, the antigen binding domain binds to CD19 and includes the scFv fragment described in Nicholson et al. Mol. Immun. 34 (16-17): 1157-1165 (1997).

In some embodiments, the antigen binding domain (for example, a humanized antigen binding domain) binds to CD19 and comprises a sequence from Table 3 of WO2014/153270, incorporated herein by reference. WO2014/153270 also describes methods of assaying the binding and efficacy of various CAR constructs.

Humanization of murine CD19 antibody is desired for the clinical setting, where the mouse-specific residues may induce a human-anti-mouse antigen (HAMA) response in patients who receive CART19 treatment, i.e., treatment with T cells transduced with the CAR19 construct. The production, characterization, and efficacy of humanized CD19 CAR sequences is described in International Application WO2014/153270 which is herein incorporated by reference in its entirety, including Examples 1-5 (p. 115-159).

In some embodiments, the antigen binding domain comprises the parental murine scFv sequence of the CAR19 construct provided in WO2012/079000 (incorporated herein by reference). In some embodiments, the antigen binding domain binds CD19 and comprises a scFv described in WO2012/079000.

BCMA

Exemplary antigen binding domains that bind BCMA are disclosed in WO2012/0163805, WO 2017/021450, WO 2017/011804, WO 2017/025038, WO 2016/090327, WO 2016/130598, WO 2016/210293, WO 2016/090320, WO 2016/014789, WO 2016/094304, WO 2016/154055, WO 2015/166073, WO 2015/188119, WO 2015/158671, U.S. Pat. Nos. 9,243,058, 8,920,776, 9,273,141, 7,083,785, 9,034,324, US 2007/0049735, US 2015/0284467, US 2015/0051266, US 2015/0344844, US 2016/0131655, US 2016/0297884, US 2016/0297885, US 2017/0051308, US 2017/0051252, US 2017/0051252, WO 2016/020332, WO 2016/087531, WO 2016/079177, WO 2015/172800, WO 2017/008169, U.S. Pat. No. 9,340,621, US 2013/0273055, US 2016/0176973, US 2015/0368351, US 2017/0051068, US 2016/0368988, and US 2015/0232557, herein incorporated by reference in their entirety. In some embodiments, the antigen binding domain of one or more of the BCMA antigen binding domains disclosed therein.

In some embodiments, the antigen binding domain comprises a human antibody or a human antibody fragment that binds BCMA. In some embodiments, the antigen binding domain comprises one or more (for example, all three) LC CDR1, LC CDR2, and LC CDR3 of a human anti-BCMA binding domain described herein (for example, in Tables 2-14), and/or one or more (for example, all three) HC CDR1, HC CDR2, and HC CDR3 of a human anti-BCMA binding domain described herein (for example, in Tables 2-14). In some embodiments, the human anti-BCMA binding domain comprises a human VL described herein (for example, in Tables 2, 6, and 10) and/or a human VH described herein (for example, in Tables 2, 6, and 10). In some embodiments, the antigen binding domain is a scFv comprising a VL and a VH of an amino acid sequence of Tables 2, 6, and 10. In some embodiments, the antigen binding domain (for example, an scFv) comprises: a VL comprising an amino acid sequence having at least one, two or three modifications (for example, substitutions, for example, conservative substitutions) but not more than 30, 20 or 10 modifications (for example, substitutions, for example, conservative substitutions) of an amino acid sequence provided in Tables 2, 6, and 10, or a sequence with 95-99% identity with an amino acid sequence of Tables 2, 6, and 10; and/or a VH comprising an amino acid sequence having at least one, two or three modifications (for example, substitutions, for example, conservative substitutions) but not more than 30, 20 or 10 modifications (for example, substitutions, for example, conservative substitutions) of an amino acid sequence provided in Tables 2, 6, and 10, or a sequence with 95-99% identity to an amino acid sequence of Tables 2, 6, and 10.

In certain embodiments, the antigen binding domain described herein includes: (1) one, two, or three light chain (LC) CDRs chosen from:
(i) a LC CDR1 of SEQ ID NO: 54, LC CDR2 of SEQ ID NO: 55 and LC CDR3 of SEQ ID NO: 56; and/or
(2) one, two, or three heavy chain (HC) CDRs from one of the following:
   (i) a HC CDR1 of SEQ ID NO: 44, HC CDR2 of SEQ ID NO: 45 and HC CDR3 of SEQ ID NO: 84; (ii) a HC CDR1 of SEQ ID NO: 44, HC CDR2 of SEQ ID NO: 45 and HC CDR3 of SEQ ID NO: 46; (iii) a HC CDR1 of SEQ ID NO: 44, HC CDR2 of SEQ ID NO: 45 and HC CDR3 of SEQ ID NO: 68; or (iv) a HC CDR1 of SEQ ID NO: 44, HC CDR2 of SEQ ID NO: 45 and HC CDR3 of SEQ ID NO: 76.

In certain embodiments, the antigen binding domain described herein includes:
(1) one, two, or three light chain (LC) CDRs from one of the following: (i) a LC CDR1 of SEQ ID NO: 95, LC CDR2 of SEQ ID NO: 131 and LC CDR3 of SEQ ID NO: 132; (ii) a LC CDR1 of SEQ ID NO: 95, LC CDR2 of SEQ ID NO: 96 and LC CDR3 of SEQ ID NO: 97; (iii) a LC CDR1 of SEQ ID NO: 95, LC CDR2 of SEQ ID NO: 114 and LC CDR3 of SEQ ID NO: 115; or (iv) a LC CDR1 of SEQ ID NO: 95, LC CDR2 of SEQ ID NO: 114 and LC CDR3 of SEQ ID NO: 97; and/or
(2) one, two, or three heavy chain (HC) CDRs from one of the following: (i) a HC CDR1 of SEQ ID NO: 86, HC CDR2 of SEQ ID NO: 130 and HC CDR3 of SEQ ID NO: 88; (ii) a HC CDR1 of SEQ ID NO: 86, HC CDR2 of SEQ ID NO: 87 and HC CDR3 of SEQ ID NO: 88; or (iii) a HC CDR1 of SEQ ID NO: 86, HC CDR2 of SEQ ID NO: 109 and HC CDR3 of SEQ ID NO: 88.

In certain embodiments, the antigen binding domain described herein includes:
(1) one, two, or three light chain (LC) CDRs from one of the following: (i) a LC CDR1 of SEQ ID NO: 147, LC CDR2 of SEQ ID NO: 182 and LC CDR3 of SEQ ID NO: 183; (ii) a LC CDR1 of SEQ ID NO: 147, LC CDR2 of SEQ ID NO: 148 and LC CDR3 of SEQ ID NO: 149; or (iii) a LC CDR1 of SEQ ID NO: 147, LC CDR2 of SEQ ID NO: 170 and LC CDR3 of SEQ ID NO: 171; and/or
(2) one, two, or three heavy chain (HC) CDRs from one of the following: (i) a HC CDR1 of SEQ ID NO: 179, HC CDR2 of SEQ ID NO: 180 and HC CDR3 of SEQ ID NO: 181; (ii) a HC CDR1 of SEQ ID NO: 137, HC CDR2 of SEQ ID NO: 138 and HC CDR3 of SEQ ID NO: 139; or (iii) a HC CDR1 of SEQ ID NO: 160, HC CDR2 of SEQ ID NO: 161 and HC CDR3 of SEQ ID NO: 162.

In some embodiments, the HC CDR1, HC CDR2, HC CDR3, LC CDR1, LC CDR2, and LC CDR3 comprise the amino acid sequences of SEQ ID NOs: 44, 45, 84, 54, 55, and 56, respectively. In some embodiments, the HC CDR1, HC CDR2, HC CDR3, LC CDR1, LC CDR2, and LC CDR3 comprise the amino acid sequences of SEQ ID NOs: 44, 45, 46, 54, 55, and 56, respectively. In some embodiments, the HC CDR1, HC CDR2, HC CDR3, LC CDR1, LC CDR2, and LC CDR3 comprise the amino acid sequences of SEQ ID NOs: 44, 45, 68, 54, 55, and 56, respectively. In some embodiments, the HC CDR1, HC CDR2, HC CDR3, LC CDR1, LC CDR2, and LC CDR3 comprise the amino acid sequences of SEQ ID NOs: 44, 45, 76, 54, 55, and 56, respectively.

In some embodiments, the HC CDR1, HC CDR2, HC CDR3, LC CDR1, LC CDR2, and LC CDR3 comprise the amino acid sequences of SEQ ID NOs: 47, 48, 84, 57, 58, and 59, respectively. In some embodiments, the HC CDR1, HC CDR2, HC CDR3, LC CDR1, LC CDR2, and LC CDR3 comprise the amino acid sequences of SEQ ID NOs: 47, 48, 46, 57, 58, and 59, respectively. In some embodiments, the HC CDR1, HC CDR2, HC CDR3, LC CDR1, LC CDR2, and LC CDR3 comprise the amino acid sequences of SEQ ID NOs: 47, 48, 68, 57, 58, and 59, respectively. In some embodiments, the HC CDR1, HC CDR2, HC CDR3, LC CDR1, LC CDR2, and LC CDR3 comprise the amino acid sequences of SEQ ID NOs: 47, 48, 76, 57, 58, and 59, respectively.

In some embodiments, the HC CDR1, HC CDR2, HC CDR3, LC CDR1, LC CDR2, and LC CDR3 comprise the amino acid sequences of SEQ ID NOs: 49, 50, 85, 60, 58, and 56, respectively. In some embodiments, the HC CDR1, HC CDR2, HC CDR3, LC CDR1, LC CDR2, and LC CDR3 comprise the amino acid sequences of SEQ ID NOs: 49, 50, 51, 60, 58, and 56, respectively. In some embodiments, the HC CDR1, HC CDR2, HC CDR3, LC CDR1, LC CDR2, and LC CDR3 comprise the amino acid sequences of SEQ ID NOs: 49, 50, 69, 60, 58, and 56, respectively. In some embodiments, the HC CDR1, HC CDR2, HC CDR3, LC CDR1, LC CDR2, and LC CDR3 comprise the amino acid sequences of SEQ ID NOs: 49, 50, 77, 60, 58, and 56, respectively.

Other Exemplary Targets

Exemplary antigen binding domains that bind CD20 are described in WO2016/164731 and WO2018/067992, incorporated herein by reference. In some embodiments, the antigen binding domain of one or more of the CD20 antigen binding domains disclosed therein. Exemplary antigen binding domains that bind CD22 are described in WO2016/164731 and WO2018/067992, incorporated herein by reference.

In some embodiments, the antigen binding domain comprises a HC CDR1, a HC CDR2, and a HC CDR3 of any heavy chain binding domain amino acid sequences listed in Table 15. In embodiments, the antigen binding domain further comprises a LC CDR1, a LC CDR2, and a LC CDR3. In embodiments, the antigen binding domain comprises a LC CDR1, a LC CDR2, and a LC CDR3 amino acid sequences listed in Table 16.

In some embodiments, the antigen binding domain comprises one, two or all of LC CDR1, LC CDR2, and LC CDR3 of any light chain binding domain amino acid sequences listed in Table 16, and one, two or all of HC CDR1, HC CDR2, and HC CDR3 of any heavy chain binding domain amino acid sequences listed in Table 15.

Exemplary antigen binding domains that bind EGFRvIII are described in in WO2014/130657.

Exemplary antigen binding domains that bind CD123 are described in WO 2014/130635 and WO2016/028896, incorporated herein by reference.

In some embodiments, the antigen binding domain comprises a sequence from Tables 1-2 of WO2014/130635, incorporated herein by reference.

In some embodiments, the antigen binding domain comprises a sequence from Tables 2, 6, and 9 of WO2016/028896, incorporated herein by reference.

Exemplary antigen binding domains that bind CLL-1 are disclosed in WO2016/014535, incorporated herein by reference.

In some embodiments, the antigen binding domain comprises one, two three (for example, all three) heavy chain CDRs, HC CDR1, HC CDR2 and HC CDR3, from an antibody described herein (for example, an antibody described in WO2015/142675, US-2015-0283178-A1, US-2016-0046724-A1, US2014/0322212A1, US2016/0068601A1, US2016/0051651A1, US2016/0096892A1, US2014/0322275A1, or WO2015/090230, incorporated herein by reference), and/or one, two, three (for example, all three) light chain CDRs, LC CDR1, LC CDR2 and LC CDR3, from an antibody described herein (for example, an antibody described in WO2015/142675, US-2015-0283178-A1, US-2016-0046724-A1, US2014/0322212A1, US2016/0068601A1, US2016/0051651A1, US2016/0096892A1, US2014/0322275A1, or WO2015/090230, incorporated herein by reference). In some embodiments, the antigen binding domain comprises a heavy chain variable region and/or a variable light chain region of an antibody listed above.

In embodiments, the antigen binding domain is an antigen binding domain described in WO2015/142675, US-2015-0283178-A1, US-2016-0046724-A1, US2014/0322212A1, US2016/0068601A1, US2016/0051651A1, US2016/0096892A1, US2014/0322275A1, or WO2015/090230, incorporated herein by reference.

Exemplary target antigens that can be targeted using the CAR-expressing cells, include, but are not limited to, CD19, CD123, EGFRvIII, CD33, mesothelin, BCMA, and GFR ALPHA-4, among others, as described in, for example, WO2014/153270, WO 2014/130635, WO2016/028896, WO 2014/130657, WO2016/014576, WO 2015/090230, WO2016/014565, WO2016/014535, and WO2016/025880, each of which is herein incorporated by reference in its entirety.

In some embodiments, the antigen binding domain of any of the CARs described herein (for example, any of CD19, CD123, EGFRvIII, CD33, mesothelin, BCMA, and GFR ALPHA-4) comprises one, two three (for example, all three) heavy chain CDRs, HC CDR1, HC CDR2 and HC CDR3, from an antibody listed above, and/or one, two, three (for example, all three) light chain CDRs, LC CDR1, LC CDR2 and LC CDR3, from an antigen binding domain listed above. In some embodiments, the antigen binding domain comprises a heavy chain variable region and/or a variable light chain region of an antibody listed or described above.

In some embodiments, the antigen binding domain comprises one, two three (for example, all three) heavy chain CDRs, HC CDR1, HC CDR2 and HC CDR3, from an antibody listed above, and/or one, two, three (for example, all three) light chain CDRs, LC CDR1, LC CDR2 and LC CDR3, from an antibody listed above. In some embodiments, the antigen binding domain comprises a heavy chain variable region and/or a variable light chain region of an antibody listed or described above.

Bispecific CARs

In certain embodiments, the antigen binding domain is a bi- or multi-specific molecule (e.g., a multispecific antibody molecule). In some embodiments a multispecific antibody molecule is a bispecific antibody molecule. A bispecific antibody has specificity for no more than two antigens. A bispecific antibody molecule is characterized by a first immunoglobulin variable domain sequence which has binding specificity for a first epitope and a second immunoglobulin variable domain sequence that has binding specificity for a second epitope. In some embodiments the first and second epitopes are on the same antigen, e.g., the same protein (or subunit of a multimeric protein). In some embodiments the first and second epitopes overlap. In some embodiments the first and second epitopes do not overlap. In some embodiments the first and second epitopes are on different antigens, e.g., different proteins (or different subunits of a multimeric protein). In some embodiments a bispecific antibody molecule comprises a heavy chain variable domain sequence and a light chain variable domain sequence which have binding specificity for a first epitope and a heavy chain variable domain sequence and a light chain variable domain sequence which have binding specificity for a second epitope. In some embodiments a bispecific antibody molecule comprises a half antibody having binding specificity for a first epitope and a half antibody having binding specificity for a second epitope. In some embodiments a bispecific antibody molecule comprises a half antibody, or fragment thereof, having binding specificity for a first epitope and a half antibody, or fragment thereof, having binding specificity for a second epitope. In some embodiments a bispecific antibody molecule comprises a scFv, or fragment thereof, have binding specificity for a first epitope and a scFv, or fragment thereof, have binding specificity for a second epitope.

In some embodiments, the antibody molecule is a multi-specific (e.g., a bispecific or a trispecific) antibody molecule. Such molecules include bispecific fusion proteins, e.g., an expression construct containing two scFvs with a hydrophilic helical peptide linker between them and a full constant region, as described in, e.g., U.S. Pat. No. 5,637,481; minibody constructs with linked VL and VH chains further connected with peptide spacers to an antibody hinge region and CH3 region, which can be dimerized to form bispecific/multivalent molecules, as described in, e.g., U.S. Pat. No. 5,837,821; String of VH domains (or VL domains in family members) connected by peptide linkages with crosslinkable groups at the C-terminus further associated with VL domains to form a series of FVs (or scFvs), as described in, e.g., U.S. Pat. No. 5,864,019; and single chain binding polypeptides with both a VH and a VL domain linked through a peptide linker are combined into multivalent structures through non-covalent or chemical crosslinking to form, e.g., homobivalent, heterobivalent, trivalent, and tetravalent structures using both scFV or diabody type format, as described in, e.g., U.S. Pat. No. 5,869,620. The contents of the above-referenced applications are incorporated herein by reference in their entireties.

Within each antibody or antibody fragment (e.g., scFv) of a bispecific antibody molecule, the VH can be upstream or downstream of the VL. In some embodiments, the upstream antibody or antibody fragment (e.g., scFv) is arranged with its VH (VH1) upstream of its VL (VL1) and the downstream antibody or antibody fragment (e.g., scFv) is arranged with its VL (VL2) upstream of its VH (VH2), such that the overall bispecific antibody molecule has the arrangement VH1-VL1-VL2-VH2. In other embodiments, the upstream antibody or antibody fragment (e.g., scFv) is arranged with its VL (VL1) upstream of its VH (VH1) and the downstream antibody or antibody fragment (e.g., scFv) is arranged with its VH (VH2) upstream of its VL (VL2), such that the overall bispecific antibody molecule has the arrangement VL1-VH1-VH2-VL2. Optionally, a linker is disposed between the two antibodies or antibody fragments (e.g., scFvs), e.g., between VL1 and VL2 if the construct is arranged as VH1-VL1-VL2-VH2, or between VH1 and VH2 if the construct is arranged as VL1-VH1-VH2-VL2. The linker may be a linker as described herein, e.g., a (Gly4-Ser)n linker, wherein n is 1, 2, 3, 4, 5, or 6, preferably 4 (SEQ ID NO: 691). In general, the linker between the two scFvs should be long enough to avoid mispairing between the domains of the two scFvs. Optionally, a linker is disposed between the VL and VH of the first scFv. Optionally, a linker is disposed between the VL and VH of the second scFv. In constructs that have multiple linkers, any two or more of the linkers can be the same or different. Accordingly, in some embodiments, a bispecific CAR comprises VLs, VHs, and optionally one or more linkers in an arrangement as described herein.

Transmembrane Domains

With respect to the transmembrane domain, in various embodiments, a chimeric molecule as described herein (e.g., a CAR) can be designed to comprise a transmembrane domain that is attached to the extracellular domain of the chimeric molecule. A transmembrane domain can include one or more additional amino acids adjacent to the transmembrane region, e.g., one or more amino acid associated with the extracellular region of the protein from which the transmembrane was derived (e.g., 1, 2, 3, 4, 5, 6, 7, 8, 9, 10 up to 15 amino acids of the extracellular region) and/or one or more additional amino acids associated with the intracellular region of the protein from which the transmembrane protein is derived (e.g., 1, 2, 3, 4, 5, 6, 7, 8, 9, 10 up to 15 amino acids of the intracellular region). In some aspects, the transmembrane domain is one that is associated with one of the other domains of the chimeric protein (e.g., CAR) e.g., in some embodiments, the transmembrane domain may be from the same protein that the signaling domain, costimulatory domain or the hinge domain is derived from. In another aspect, the transmembrane domain is not derived from the same protein that any other domain of the chimeric protein (e.g., CAR) is derived from. In some instances, the transmembrane domain can be selected or modified by amino acid substitution to avoid binding of such domains to the transmembrane domains of the same or different surface membrane proteins, e.g., to minimize interactions with other members of the receptor complex. In some aspects, the transmembrane domain is capable of homodimerization with another CAR on the cell surface of a CAR-expressing cell. In a different aspect, the amino acid sequence of the transmembrane domain may be modified or substituted so as to minimize interactions with the binding domains of the native binding partner present in the same CAR-expressing cell.

The transmembrane domain may be derived either from a natural or from a recombinant source. Where the source is natural, the domain may be derived from any membrane-bound or transmembrane protein. In some aspects the transmembrane domain is capable of signaling to the intracellular domain(s) whenever the CAR has bound to a target. A transmembrane domain of particular use in this invention may include at least the transmembrane region(s) of e.g., the alpha, beta or zeta chain of the T-cell receptor, CD28, CD27, CD3 epsilon, CD45, CD4, CD5, CD8, CD9, CD16, CD22, CD33, CD37, CD64, CD80, CD86, CD134, CD137, CD154. In some embodiments, a transmembrane domain may include at least the transmembrane region(s) of, e.g., KIRDS2, OX40, CD2, CD27, LFA-1 (CD11a, CD18), ICOS (CD278), 4-1BB (CD137), GITR, CD40, BAFFR, HVEM (LIGHTR), SLAMF7, NKp80 (KLRF1), NKp44, NKp30, NKp46, CD160, CD19, IL2R beta, IL2R gamma, IL7R a, ITGA1, VLA1, CD49a, ITGA4, IA4, CD49D, ITGA6, VLA-6, CD49f, ITGAD, CD11 d, ITGAE, CD103, ITGAL, CD11 a, LFA-1, ITGAM, CD11b, ITGAX, CD11 c, ITGB1, CD29, ITGB2, CD18, LFA-1, ITGB7, TNFR2, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), SLAMF6 (NTB-A, Ly108), SLAM (SLAMF1, CD150, IPO-3), BLAME (SLAMF8), SELPLG (CD162), LTBR, PAG/Cbp, NKG2D, or NKG2C.

In some instances, the transmembrane domain can be attached to the extracellular region of the CAR, e.g., the antigen binding domain of the CAR, via a hinge, e.g., a hinge from a human protein. For example, in some embodiments, the hinge can be a human Ig (immunoglobulin) hinge (e.g., an IgG4 hinge, an IgD hinge), a GS linker (e.g., a GS linker described herein), a KIR2DS2 hinge or a CD8a hinge. In some embodiments, the hinge or spacer comprises (e.g., consists of) the amino acid sequence of SEQ ID NO:4. In some aspects, the transmembrane domain comprises (e.g., consists of) a transmembrane domain of SEQ ID NO: 12.

In some embodiments, the encoded transmembrane domain comprises an amino acid sequence of a CD8 transmembrane domain having at least one, two or three modifications but not more than 20, 10 or 5 modifications of an amino acid sequence of SEQ ID NO: 12, or a sequence with 95-99% identity to an amino acid sequence of SEQ ID NO: 12. In some embodiments, the encoded transmembrane domain comprises the sequence of SEQ ID NO: 12.

In other embodiments, the nucleic acid molecule encoding the CAR comprises a nucleotide sequence of a CD8 transmembrane domain, e.g., comprising the sequence of SEQ ID NO: 13, or a sequence with 95-99% identity thereof.

In some embodiments, the encoded antigen binding domain is connected to the transmembrane domain by a hinge region. In some embodiments, the encoded hinge region comprises the amino acid sequence of a CD8 hinge, e.g., SEQ ID NO: 4; or the amino acid sequence of an IgG4 hinge, e.g., SEQ ID NO: 6, or a sequence with 95-99% identity to SEQ ID NO:4 or 6. In other embodiments, the nucleic acid sequence encoding the hinge region comprises a sequence of SEQ ID NO: 5 or SEQ ID NO: 7, corresponding to a CD8 hinge or an IgG4 hinge, respectively, or a sequence with 95-99% identity to SEQ ID NO:5 or 7.

In some aspects, the hinge or spacer comprises an IgG4 hinge. For example, in some embodiments, the hinge or spacer comprises a hinge of the amino acid sequence ESKYGPPCPPCPAPEFLGGPSVFLFPPKPKDTLMISRTPEVTCVVVDVSQEDPEVQFNWY VDGVEVHNAKTKPREEQFNSTYRVVSVLTVLHQDWLNGKEYKCKVSNKGLPSSIEKTIS KAKGQPREPQVYTLPPSQEEMTKNQVSLTCLVKGFYPSDIAVEWESNGQPENNYKTTPP VLDSDGSFFLYSRLTVDKSRWQEGNVFSCSVMHEALHNHYTQKSLSLSLGKM (SEQ ID NO:6). In some embodiments, the hinge or spacer comprises a hinge encoded by a nucleotide sequence of

(SEQ ID NO: 7)
GAGAGCAAGTACGGCCCTCCCTGCCCCCCTTGCCCTGCCCCCGAGTTCCT
GGGCGGACCCAGCGTGTTCCTGTTCCCCCCCAAGCCCAAGGACACCCTGA
TGATCAGCCGGACCCCCGAGGTGACCTGTGTGGTGGTGGACGTGTCCCAG
GAGGACCCCGAGGTCCAGTTCAACTGGTACGTGGACGGCGTGGAGGTGCA
CAACGCCAAGACCAAGCCCCGGGAGGAGCAGTTCAATAGCACCTACCGGG
TGGTGTCCGTGCTGACCGTGCTGCACCAGGACTGGCTGAACGGCAAGGAA
TACAAGTGTAAGGTGTCCAACAAGGGCCTGCCCAGCAGCATCGAGAAAAC
CATCAGCAAGGCCAAGGGCCAGCCTCGGGAGCCCCAGGTGTACACCCTGC
CCCCTAGCCAAGAGGAGATGACCAAGAACCAGGTGTCCCTGACCTGCCTG
GTGAAGGGCTTCTACCCCAGCGACATCGCCGTGGAGTGGGAGAGCAACGG
CCAGCCCGAGAACAACTACAAGACCACCCCCCCTGTGCTGGACAGCGACG
GCAGCTTCTTCCTGTACAGCCGGCTGACCGTGGACAAGAGCCGGTGGCAG
GAGGGCAACGTCTTTAGCTGCTCCGTGATGCACGAGGCCCTGCACAACCA
CTACACCCAGAAGAGCCTGAGCCTGTCCCTGGGCAAGATG.

In some aspects, the hinge or spacer comprises an IgD hinge. For example, in some embodiments, the hinge or spacer comprises a hinge of the amino acid sequence RWPESPKAQASSVPTAQPQAEGSLAKATTAPATTRNTGRGGEEKKKEKEKEEQEERETK TPECPSHTQPLGVYLLTPAVQDLWLRDKATFTCFVVGSDLKDAHLTWEVAGKVPTGGV EEGLLERHSNGSQSQHSRLTLPRSLWNAGTSVTCTLNHPSLPPQRLMALREPAAQAPVK LSLNLLASSDPPEAASWLLCEVSGFSPPNILLMWLEDQREVNTSGFAPARPPPQPGSTTF WAWSVLRVPAPPSPQPATYTCVVSHEDSRTLLNASRSLEVSYVTDH (SEQ ID NO:8). In some embodiments, the hinge or spacer comprises a hinge encoded by a nucleotide sequence of

(SEQ ID NO: 9)
AGGTGGCCCGAAAGTCCCAAGGCCCAGGCATCTAGTGTTCCTACTGCACA
GCCCCAGGCAGAAGGCAGCCTAGCCAAAGCTACTACTGCACCTGCCACTA
CGCGCAATACTGGCCGTGGCGGGGAGGAGAAGAAAAAGGAGAAAGAGAAA
GAAGAACAGGAAGAGAGGGAGACCAAGACCCCTGAATGTCCATCCCATAC
CCAGCCGCTGGGCGTCTATCTCTTGACTCCCGCAGTACAGGACTTGTGGC
TTAGAGATAAGGCCACCTTTACATGTTTCGTCGTGGGCTCTGACCTGAAG
GATGCCCATTTGACTTGGGAGGTTGCCGGAAAGGTACCCACAGGGGGGGT
TGAGGAAGGGTTGCTGGAGCGCCATTCCAATGGCTCTCAGAGCCAGCACT
CAAGACTCACCCTTCCGAGATCCCTGTGGAACGCCGGGACCTCTGTCACA
TGTACTCTAAATCATCCTAGCCTGCCCCCACAGCGTCTGATGGCCCTTAG
AGAGCCAGCCGCCCAGGCACCAGTTAAGCTTAGCCTGAATCTGCTCGCCA
GTAGTGATCCCCCAGAGGCCGCCAGCTGGCTCTTATGCGAAGTGTCCGGC
TTTAGCCCGCCCAACATCTTGCTCATGTGGCTGGAGGACCAGCGAGAAGT
GAACACCAGCGGCTTCGCTCCAGCCCGGCCCCCACCCCAGCCGGGTTCTA
CCACATTCTGGGCCTGGAGTGTCTTAAGGGTCCCAGCACCACCTAGCCCC
CAGCCAGCCACATACACCTGTGTTGTGTCCCATGAAGATAGCAGGACCCT
GCTAAATGCTTCTAGGAGTCTGGAGGTTTCCTACGTGACTGACCATT

In some aspects, the transmembrane domain may be recombinant, in which case it will comprise predominantly hydrophobic residues such as leucine and valine. In some aspects a triplet of phenylalanine, tryptophan and valine can be found at each end of a recombinant transmembrane domain.

Optionally, a short oligo- or polypeptide linker, between 2 and 10 amino acids in length may form the linkage between the transmembrane domain and the cytoplasmic region of the CAR. A glycine-serine doublet provides a particularly suitable linker. For example, in some aspects, the linker comprises the amino acid sequence of GGGGSGGGGS (SEQ ID NO:10). In some embodiments, the linker is encoded by a nucleotide sequence of GGTGGCGGAGGTTCTGGAGGTGGAGGTTCC (SEQ ID NO: 11). In some embodiments, the linker comprises the amino acid sequence of GGGGS (SEQ ID NO: 877). In some embodiments the linker is encoded by a nucleotide sequence of SEQ ID NO: 876).

In some aspects, the hinge or spacer comprises a KIR2DS2 hinge.

Signaling Domains

In embodiments of the invention having an intracellular signaling domain, such a domain can contain, e.g., one or more of a primary signaling domain and/or a costimulatory signaling domain. In some embodiments, the intracellular signaling domain comprises a sequence encoding a primary signaling domain. In some embodiments, the intracellular signaling domain comprises a costimulatory signaling domain. In some embodiments, the intracellular signaling domain comprises a primary signaling domain and a costimulatory signaling domain.

The intracellular signaling sequences within the cytoplasmic portion of the CAR of the invention may be linked to each other in a random or specified order. Optionally, a short oligo-or polypeptide linker, for example, between 2 and 10 amino acids (e.g., 2, 3, 4, 5, 6, 7, 8, 9, or 10 amino acids) in length may form the linkage between intracellular signaling sequences. In some embodiments, a glycine-serine doublet can be used as a suitable linker. In some embodiments, a single amino acid, e.g., an alanine, a glycine, can be used as a suitable linker.

In some aspects, the intracellular signaling domain is designed to comprise two or more, e.g., 2, 3, 4, 5, or more, costimulatory signaling domains. In some embodiments, the two or more, e.g., 2, 3, 4, 5, or more, costimulatory signaling domains, are separated by a linker molecule, e.g., a linker molecule described herein. In some embodiments, the intracellular signaling domain comprises two costimulatory signaling domains. In some embodiments, the linker molecule is a glycine residue. In some embodiments, the linker is an alanine residue.

Primary Signaling Domains

A primary signaling domain regulates primary activation of the TCR complex either in a stimulatory way, or in an inhibitory way. Primary intracellular signaling domains that act in a stimulatory manner may contain signaling motifs which are known as immunoreceptor tyrosine-based activation motifs or ITAMs. In CARs such domains are used for the same purpose. Examples of ITAM containing primary intracellular signaling domains that are of particular use in the invention include those of CD3 zeta, common FcR gamma (FCERIG), Fc gamma RIIa, FcR beta (Fc Epsilon Rib), CD3 gamma, CD3 delta, CD3 epsilon, CD79a, CD79b, DAP10, and DAP12. In some embodiments, a CAR of the invention comprises an intracellular signaling domain, e.g., a primary signaling domain of CD3-zeta.

In some embodiments, the encoded primary signaling domain comprises a functional signaling domain of CD3 zeta. The encoded CD3 zeta primary signaling domain can comprise an amino acid sequence having at least one, two or three modifications but not more than 20, 10 or 5 modifications of an amino acid sequence of SEQ ID NO: 18 or SEQ ID NO: 20, or a sequence with 95-99% identity to an amino acid sequence of SEQ ID NO: 18 or SEQ ID NO: 20. In some embodiments, the encoded primary signaling domain comprises a sequence of SEQ ID NO:18 or SEQ ID NO: 20. In other embodiments, the nucleic acid sequence encoding the primary signaling domain comprises a sequence of SEQ ID NO:19 or SEQ ID NO: 21, or a sequence with 95-99% identity thereof.

Costimulatory Signaling Domains

In some embodiments, the encoded intracellular signaling domain comprises a costimulatory signaling domain. For example, the intracellular signaling domain can comprise a primary signaling domain and a costimulatory signaling domain. In some embodiments, the encoded costimulatory signaling domain comprises a functional signaling domain of a protein chosen from one or more of CD27, CD28, 4-1BB (CD137), OX40, CD30, CD40, PD-1, ICOS, lymphocyte function-associated antigen-1 (LFA-1), CD2, CD7, LIGHT, NKG2C, B7-H3, a ligand that specifically binds with CD83, CDS, ICAM-1, GITR, BAFFR, HVEM (LIGHTR), SLAMF7, NKp80 (KLRF1), CD160, CD19, CD4, CD8alpha, CD8beta, IL2R beta, IL2R gamma, IL7R alpha, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, TNFR2, TRANCE/RANKL, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, NKp44, NKp30, NKp46, and NKG2D.

In some embodiments, the encoded costimulatory signaling domain comprises an amino acid sequence having at least one, two or three modifications but not more than 20, 10 or 5 modifications of an amino acid sequence of SEQ ID NO:14 or SEQ ID NO: 16, or a sequence with 95-99% identity to an amino acid sequence of SEQ ID NO:14 or SEQ ID NO: 16. In some embodiments, the encoded costimulatory signaling domain comprises a sequence of SEQ ID NO: 14 or SEQ ID NO: 16. In other embodiments, the nucleic acid sequence encoding the costimulatory signaling domain comprises a sequence of SEQ ID NO:15 or SEQ ID NO: 17, or a sequence with 95-99% identity thereof.

In other embodiments, the encoded intracellular domain comprises the sequence of SEQ ID NO: 14 or SEQ ID NO: 16, and the sequence of SEQ ID NO: 18 or SEQ ID NO: 20, wherein the sequences comprising the intracellular signaling domain are expressed in the same frame and as a single polypeptide chain.

In some embodiments, the nucleic acid sequence encoding the intracellular signaling domain comprises a sequence of SEQ ID NO:15 or SEQ ID NO: 17, or a sequence with 95-99% identity thereof, and a sequence of SEQ ID NO:19 or SEQ ID NO:21, or a sequence with 95-99% identity thereof.

In some embodiments, the nucleic acid molecule further encodes a leader sequence. In some embodiments, the leader sequence comprises the sequence of SEQ ID NO: 2.

In some aspects, the intracellular signaling domain is designed to comprise the signaling domain of CD3-zeta and the signaling domain of CD28. In some aspects, the intracellular signaling domain is designed to comprise the signaling domain of CD3-zeta and the signaling domain of 4-1BB. In some aspects, the signaling domain of 4-1BB is a signaling domain of SEQ ID NO: 14. In some aspects, the signaling domain of CD3-zeta is a signaling domain of SEQ ID NO: 18.

In some aspects, the intracellular signaling domain is designed to comprise the signaling domain of CD3-zeta and the signaling domain of CD27. In some aspects, the signaling domain of CD27 comprises an amino acid sequence of QRRKYRSNKGESPVEPAEPCRYSCPREEEGSTIPIQEDYRKPEPACSP (SEQ ID NO:16). In some aspects, the signaling domain of CD27 is encoded by a nucleic acid sequence of

(SEQ ID NO: 17)
AGGAGTAAGAGGAGCAGGCTCCTGCACAGTGACTACATGAACATGACTCC
CCGCCGCCCCGGGCCCACCCGCAAGCATTACCAGCCCTATGCCCCACCAC
GCGACTTCGCAGCCTATCGCTCC

Inhibitory Domains

In some embodiments, the vector comprises a nucleic acid sequence that encodes a CAR, e.g., a CAR described herein, and a nucleic acid sequence that encodes an inhibitory molecule comprising: an inhKIR cytoplasmic domain; a transmembrane domain, e.g., a KIR transmembrane domain; and an inhibitor cytoplasmic domain, e.g., an ITIM domain, e.g., an inhKIR ITIM domain. In some embodiments the inhibitory molecule is a naturally occurring inhKIR, or a sequence sharing at least 50, 60, 70, 80, 85, 90, 95, or 99% homology with, or that differs by no more than 1, 2, 3, 4, 5, 6, 7, 8, 9, 10, 15, or 20 residues from, a naturally occurring inhKIR.

In some embodiments, the nucleic acid sequence that encodes an inhibitory molecule comprises: a SLAM family cytoplasmic domain; a transmembrane domain, e.g., a SLAM family transmembrane domain; and an inhibitor cytoplasmic domain, e.g., a SLAM family domain, e.g., an SLAM family ITIM domain. In some embodiments the inhibitory molecule is a naturally occurring SLAM family member, or a sequence sharing at least 50, 60, 70, 80, 85, 90, 95, or 99% homology with, or that differs by no more than 1, 2, 3, 4, 5, 6, 7, 8, 9, 10, 15, or 20 residues from, a naturally occurring SLAM family member.

In some embodiments, the vector is an in vitro transcribed vector, e.g., a vector that transcribes RNA of a nucleic acid molecule described herein. In some embodiments, the nucleic acid sequence in the vector further comprises a poly(A) tail, e.g., a poly A tail. In some embodiments, the nucleic acid sequence in the vector further comprises a 3′UTR, e.g., a 3′ UTR described herein, e.g., comprising at least one repeat of a 3′UTR derived from human beta-globulin. In some embodiments, the nucleic acid sequence in the vector further comprises promoter, e.g., a T2A promoter.

Promoters

In some embodiments, the vector further comprises a promoter. In some embodiments, the promoter is chosen from an EF-1 promoter, a CMV IE gene promoter, an EF-1a promoter, an ubiquitin C promoter, or a phosphoglycerate kinase (PGK) promoter. In some embodiments, the promoter is an EF-1 promoter. In some embodiments, the EF-1 promoter comprises a sequence of SEQ ID NO: 1.

In some aspects of the present invention, immune effector cells, e.g., T cells, can be obtained from a unit of blood collected from a subject using any number of techniques known to the skilled artisan, such as Ficoll™ separation. In some aspects, cells from the circulating blood of an individual are obtained by apheresis. The apheresis product typically contains lymphocytes, including T cells, monocytes, granulocytes, B cells, other nucleated white blood cells, red blood cells, and platelets. In some aspects, the cells collected by apheresis may be washed to remove the plasma fraction and, optionally, to suspend the cells in a buffer or medium for subsequent processing steps. In some embodiments, the cells are washed with phosphate buffered saline (PBS). In an alternative embodiment, the wash solution lacks calcium and may lack magnesium or may lack many if not all divalent cations.

TABLE 17
Sequences of various components of CAR (aa - amino acids, na - nucleic acids
that encodes the corresponding protein)

| SEQ | | |
|---|---|---|
| ID | | |
| NO | description | Sequence |
| 1 | EF-1 | CGTGAGGCTCCGGTGCCCGTCAGTGGGCAGAGCGCACATCG |
| | promoter | CCCACAGTCCCCGAGAAGTTGGGGGGAGGGGTCGGCAATTG |
| | | AACCGGTGCCTAGAGAAGGTGGCGCGGGGTAAACTGGGAAA |
| | | GTGATGTCGTGTACTGGCTCCGCCTTTTTCCCGAGGGTGGGG |
| | | GAGAACCGTATATAAGTGCAGTAGTCGCCGTGAACGTTCTTT |
| | | TTCGCAACGGGTTTGCCGCCAGAACACAGGTAAGTGCCGTGT |
| | | GTGGTTCCCGCGGGCCTGGCCTCTTTACGGGTTATGGCCCTT |
| | | GCGTGCCTTGAATTACTTCCACCTGGCTGCAGTACGTGATTC |
| | | TTGATCCCGAGCTTCGGGTTGGAAGTGGGTGGGAGAGTTCG |
| | | AGGCCTTGCGCTTAAGGAGCCCCTTCGCCTCGTGCTTGAGTT |
| | | GAGGCCTGGCCTGGGCGCTGGGGCCGCCGCGTGCGAATCTG |
| | | GTGGCACCTTCGCGCCTGTCTCGCTGCTTTCGATAAGTCTCTA |
| | | GCCATTTAAAATTTTTGATGACCTGCTGCGACGCTTTTTTTCT |
| | | GGCAAGATAGTCTTGTAAATGCGGGCCAAGATCTGCACACT |
| | | GGTATTTCGGTTTTTGGGGCCGCGGGCGGCGACGGGGCCCGT |
| | | GCGTCCCAGCGCACATGTTCGGCGAGGCGGGGCCTGCGAGC |
| | | GCGGCCACCGAGAATCGGACGGGGGTAGTCTCAAGCTGGCC |
| | | GGCCTGCTCTGGTGCCTGGCCTCGCGCCGCCGTGTATCGCCC |
| | | CGCCCTGGGCGGCAAGGCTGGCCCGGTCGGCACCAGTTGCG |
| | | TGAGCGGAAAGATGGCCGCTTCCCGGCCCTGCTGCAGGGAG |
| | | CTCAAAATGGAGGACGCGGCGCTCGGGAGAGCGGGCGGGTG |
| | | AGTCACCCACACAAAGGAAAAGGGCCTTTCCGTCCTCAGCC |
| | | GTCGCTTCATGTGACTCCACGGAGTACCGGGCGCCGTCCAGG |
| | | CACCTCGATTAGTTCTCGAGCTTTTGGAGTACGTCGTCTTTAG |
| | | GTTGGGGGGAGGGGTTTTATGCGATGGAGTTTCCCCACACTG |
| | | AGTGGGTGGAGACTGAAGTTAGGCCAGCTTGGCACTTGATG |
| | | TAATTCTCCTTGGAATTTGCCCTTTTTGAGTTTGGATCTTGGT |
| | | TCATTCTCAAGCCTCAGACAGTGGTTCAAAGTTTTTTTCTTCC |
| | | ATTTCAGGTGTCGTGA |
| 2 | Leader (aa) | MALPVTALLLPLALLLHAARP |
| 3 | Leader (na) | ATGGCCCTGCCTGTGACAGCCCTGCTGCTGCCTCTGGCTCTG |
| | | CTGCTGCATGCCGCTAGACCC |
| 4 | CD 8 hinge | TTTPAPRPPTPAPTIASQPLSLRPEACRPAAGGAVHTRGLDFACD |
| | (aa) | |
| 5 | CD8 hinge | ACCACGACGCCAGCGCCGCGACCACCAACACCGGCGCCCAC |
| | (na) | CATCGCGTCGCAGCCCCTGTCCCTGCGCCCAGAGGCGTGCCG |
| | | GCCAGCGGCGGGGGGCGCAGTGCACACGAGGGGGCTGGACT |
| | | TCGCCTGTGAT |
| 6 | Ig4 hinge | ESKYGPPCPPCPAPEFLGGPSVFLFPPKPKDTLMISRTPEVTCVV |
| | (aa) | VDVSQEDPEVQFNWYVDGVEVHNAKTKPREEQFNSTYRVVSV |
| | | LTVLHQDWLNGKEYKCKVSNKGLPSSIEKTISKAKGQPREPQV |
| | | YTLPPSQEEMTKNQVSLTCLVKGFYPSDIAVEWESNGQPENNY |
| | | KTTPPVLDSDGSFFLYSRLTVDKSRWQEGNVFSCSVMHEALHN |
| | | HYTQKSLSLSLGKM |
| 7 | Ig4 hinge | GAGAGCAAGTACGGCCCTCCCTGCCCCCCTTGCCCTGCCCCC |
| | (na) | GAGTTCCTGGGCGGACCCAGCGTGTTCCTGTTCCCCCCCAAG |
| | | CCCAAGGACACCCTGATGATCAGCCGGACCCCCGAGGTGAC |
| | | CTGTGTGGTGGTGGACGTGTCCCAGGAGGACCCCGAGGTCC |
| | | AGTTCAACTGGTACGTGGACGGCGTGGAGGTGCACAACGCC |
| | | AAGACCAAGCCCCGGGAGGAGCAGTTCAATAGCACCTACCG |
| | | GGTGGTGTCCGTGCTGACCGTGCTGCACCAGGACTGGCTGAA |
| | | CGGCAAGGAATACAAGTGTAAGGTGTCCAACAAGGGCCTGC |
| | | CCAGCAGCATCGAGAAAACCATCAGCAAGGCCAAGGGCCAG |
| | | CCTCGGGAGCCCCAGGTGTACACCCTGCCCCCTAGCCAAGA |
| | | GGAGATGACCAAGAACCAGGTGTCCCTGACCTGCCTGGTGA |
| | | AGGGCTTCTACCCCAGCGACATCGCCGTGGAGTGGGAGAGC |
| | | AACGGCCAGCCCGAGAACAACTACAAGACCACCCCCCCTGT |
| | | GCTGGACAGCGACGGCAGCTTCTTCCTGTACAGCCGGCTGAC |
| | | CGTGGACAAGAGCCGGTGGCAGGAGGGCAACGTCTTTAGCT |
| | | GCTCCGTGATGCACGAGGCCCTGCACAACCACTACACCCAG |
| | | AAGAGCCTGAGCCTGTCCCTGGGCAAGATG |
| 8 | IgD hinge | RWPESPKAQASSVPTAQPQAEGSLAKATTAPATTRNTGRGGEE |
| | (aa) | KKKEKEKEEQEERETKTPECPSHTQPLGVYLLTPAVQDLWLRD |
| | | KATFTCFVVGSDLKDAHLTWEVAGKVPTGGVEEGLLERHSNG |
| | | SQSQHSRLTLPRSLWNAGTSVTCTLNHPSLPPQRLMALREPAA |
| | | QAPVKLSLNLLASSDPPEAASWLLCEVSGFSPPNILLMWLEDQR |
| | | EVNTSGFAPARPPPQPGSTTFWAWSVLRVPAPPSPQPATYTCVV |
| | | SHEDSRTLLNASRSLEVSYVTDH |
| 9 | IgD hinge | AGGTGGCCCGAAAGTCCCAAGGCCCAGGCATCTAGTGTTCCT |
| | (na) | ACTGCACAGCCCCAGGCAGAAGGCAGCCTAGCCAAAGCTAC |
| | | TACTGCACCTGCCACTACGCGCAATACTGGCCGTGGCGGGG |
| | | AGGAGAAGAAAAAGGAGAAAGAGAAAGAAGAACAGGAAG |
| | | AGAGGGAGACCAAGACCCCTGAATGTCCATCCCATACCCAG |
| | | CCGCTGGGCGTCTATCTCTTGACTCCCGCAGTACAGGACTTG |
| | | TGGCTTAGAGATAAGGCCACCTTTACATGTTTCGTCGTGGGC |
| | | TCTGACCTGAAGGATGCCCATTTGACTTGGGAGGTTGCCGGA |
| | | AAGGTACCCACAGGGGGGGTTGAGGAAGGGTTGCTGGAGCG |
| | | CCATTCCAATGGCTCTCAGAGCCAGCACTCAAGACTCACCCT |
| | | TCCGAGATCCCTGTGGAACGCCGGGACCTCTGTCACATGTAC |
| | | TCTAAATCATCCTAGCCTGCCCCCACAGCGTCTGATGGCCCT |
| | | TAGAGAGCCAGCCGCCCAGGCACCAGTTAAGCTTAGCCTGA |
| | | ATCTGCTCGCCAGTAGTGATCCCCCAGAGGCCGCCAGCTGGC |
| | | TCTTATGCGAAGTGTCCGGCTTTAGCCCGCCCAACATCTTGC |
| | | TCATGTGGCTGGAGGACCAGCGAGAAGTGAACACCAGCGGC |
| | | TTCGCTCCAGCCCGGCCCCCACCCCAGCCGGGTTCTACCACA |
| | | TTCTGGGCCTGGAGTGTCTTAAGGGTCCCAGCACCACCTAGC |
| | | CCCCAGCCAGCCACATACACCTGTGTTGTGTCCCATGAAGAT |
| | | AGCAGGACCCTGCTAAATGCTTCTAGGAGTCTGGAGGTTTCC |
| | | TACGTGACTGACCATT |
| 10 | GS | GGGGSGGGGS |
| | hinge/linker | |
| | (aa) | |
| 11 | GS | GGTGGCGGAGGTTCTGGAGGTGGAGGTTCC |
| | hinge/linker | |
| | (na) | |
| 12 | CD8 TM | IYIWAPLAGTCGVLLLSLVITLYC |
| | (aa) | |
| 13 | CD8 TM | ATCTACATCTGGGCGCCCTTGGCCGGGACTTGTGGGGTCCTT |
| | (na) | CTCCTGTCACTGGTTATCACCCTTTACTGC |
| 14 | 4-1BB | KRGRKKLLYIFKQPFMRPVQTTQEEDGCSCRFPEEEEGGCEL |
| | intracellular | |
| | domain (aa) | |
| 15 | 4-1BB | AAACGGGGCAGAAAGAAACTCCTGTATATATTCAAACAACC |
| | intracellular | ATTTATGAGACCAGTACAAACTACTCAAGAGGAAGATGGCT |
| | domain (na) | GTAGCTGCCGATTTCCAGAAGAAGAAGAAGGAGGATGTGAA |
| | | CTG |
| 16 | CD27 (aa) | QRRKYRSNKGESPVEPAEPCRYSCPREEEGSTIPIQEDYRKPEPA |
| | | CSP |
| 17 | CD27 (na) | AGGAGTAAGAGGAGCAGGCTCCTGCACAGTGACTACATGAA |
| | | CATGACTCCCCGCCGCCCCGGGCCCACCCGCAAGCATTACCA |
| | | GCCCTATGCCCCACCACGCGACTTCGCAGCCTATCGCTCC |
| 18 | CD3-zeta | RVKFSRSADAPAYKQGQNQLYNELNLGRREEYDVLDKRRGRD |
| | (aa) | PEMGGKPRRKNPQEGLYNELQKDKMAEAYSEIGMKGERRRGK |
| | | GHDGLYQGLSTATKDTYDALHMQALPPR |
| 19 | CD3-zeta | AGAGTGAAGTTCAGCAGGAGCGCAGACGCCCCCGCGTACAA |
| | (na) | GCAGGGCCAGAACCAGCTCTATAACGAGCTCAATCTAGGAC |
| | | GAAGAGAGGAGTACGATGTTTTGGACAAGAGACGTGGCCGG |
| | | GACCCTGAGATGGGGGGAAAGCCGAGAAGGAAGAACCCTC |
| | | AGGAAGGCCTGTACAATGAACTGCAGAAAGATAAGATGGCG |
| | | GAGGCCTACAGTGAGATTGGGATGAAAGGCGAGCGCCGGAG |
| | | GGGCAAGGGGCACGATGGCCTTTACCAGGGTCTCAGTACAG |
| | | CCACCAAGGACACCTACGACGCCCTTCACATGCAGGCCCTG |
| | | CCCCCTCGC |
| 20 | CD3-zeta | RVKFSRSADAPAYQQGQNQLYNELNLGRREEYDVLDKRRGRD |
| | (aa) | PEMGGKPRRKNPQEGLYNELQKDKMAEAYSEIGMKGERRRGK |
| | | GHDGLYQGLSTATKDTYDALHMQALPPR |
| 21 | CD3-zeta | AGAGTGAAGTTCAGCAGGAGCGCAGACGCCCCCGCGTACCA |
| | (na) | GCAGGGCCAG |
| | | AACCAGCTCTATAACGAGCTCAATCTAGGACGAAGAGAGGA |
| | | GTACGATGTTT |
| | | TGGACAAGAGACGTGGCCGGGACCCTGAGATGGGGGGAAA |
| | | GCCGAGAAGGA |
| | | AGAACCCTCAGGAAGGCCTGTACAATGAACTGCAGAAAGAT |
| | | AAGATGGCGG |
| | | AGGCCTACAGTGAGATTGGGATGAAAGGCGAGCGCCGGAGG |
| | | GGCAAGGGGC |
| | | ACGATGGCCTTTACCAGGGTCTCAGTACAGCCACCAAGGAC |
| | | ACCTACGACGC |
| | | CCTTCACATGCAGGCCCTGCCCCCTCGC |
| 22 | linker | GGGGS |
| 23 | linker | GGTGGCGGAGGTTCTGGAGGTGGAGGTTCC |
| 24 | PD-1 | Pgwfldspdrpwnpptfspallvvtegdnatftcsfsntsesfvlnwyrmspsnqtdklaafpedr |
| | extracellular | sqpgqdcrfrvtqlpngrdfhmsvvrarrndsgtylcgaislapkaqikeslraelrvterraevpta |
| | domain (aa) | hpspsprpagqfqtlv |
| 25 | PD-1 | Cccggatggtttctggactctccggatcgcccgtggaatcccccaaccttctcaccggcactcttggt |
| | extracellular | tgtgactgagggcgataatgcgaccttcacgtgctcgttctccaacacctccgaatcattcgtgctgaa |
| | domain (na) | ctggtaccgcatgagcccgtcaaaccagaccgacaagctcgccgcgtttccggaagatcggtcgca |
| | | accgggacaggattgtcggttccgcgtgactcaactgccgaatggcagagacttccacatgagcgt |
| | | ggtccgcgctaggcgaaacgactccgggacctacctgtgcggagccatctcgctggcgcctaagg |
| | | cccaaatcaaagagagcttgagggccgaactgagagtgaccgagcgcagagctgaggtgccaact |
| | | gcacatccatccccatcgcctcggcctgcggggcagtttcagaccctggtc |
| 26 | PD-1 CAR | Malpvtalllplalllhaarppgwfldspdrpwnpptfspallvvtegdnatftcsfsntsesfvlnw |
| | (aa) with | yrmspsnqtdklaafpedrsqpgqdcrfrvtqlpngrdfhmsvvrarrndsgtylcgaislapka |
| | signal | qikeslraelrvterraevptahpspsprpagqfqtlvtttpaprpptpaptiasqplslrpeacrpaag |
| | | gavhtrgldfacdiyiwaplagtcgvlllslvitlyckrgrkkllyifkqpfmrpvqttqeedgcscrf |
| | | peeeeggcelrvkfsrsadapaykqgqnqlynelnlgrreeydvldkrrgrdpemggkprrknp |
| | | geglynelqkdkmaeayseigmkgerrrgkghdglyqglstatkdtydalhmqalppr |
| 27 | PD-1 CAR | Atggccctccctgtcactgccctgcttctccccctegcactcctgctccacgccgctagaccacccg |
| | (na) | gatggtttctggactctccggatcgcccgtggaatcccccaaccttctcaccggcactcttggttgtga |
| | | ctgagggcgataatgcgaccttcacgtgctcgttctccaacacctccgaatcattcgtgctgaactggt |
| | | accgcatgagcccgtcaaaccagaccgacaagctcgccgcgtttccggaagatcggtcgcaaccg |
| | | ggacaggattgtcggttccgcgtgactcaactgccgaatggcagagacttccacatgagcgtggtcc |
| | | gogctaggcgaaacgactccgggacctacctgtgcggagccatctcgctggcgcctaaggcccaa |
| | | atcaaagagagcttgagggccgaactgagagtgaccgagcgcagagctgaggtgccaactgcac |
| | | atccatccccatcgcctcggcctgcggggcagtttcagaccctggtcacgaccactccggcgccgc |
| | | gcccaccgactccggccccaactatcgcgagccagcccctgtcgctgaggccggaagcatgccgc |
| | | cctgccgccggaggtgctgtgcatacccggggattggacttcgcatgcgacatctacatttgggctcc |
| | | tctcgccggaacttgtggcgtgctccttctgtccctggtcatcaccctgtactgcaagcggggtcgga |
| | | aaaagcttctgtacattttcaagcagcccttcatgaggcccgtgcaaaccacccaggaggaggacgg |
| | | ttgctcctgccggttccccgaagaggaagaaggaggttgcgagctgcgcgtgaagttctcccggag |
| | | cgccgacgcccccgcctataagcagggccagaaccagctgtacaacgaactgaacctgggacgg |
| | | cgggaagagtacgatgtgctggacaagcggcgcggccgggaccccgaaatgggcgggaagcct |
| | | agaagaaagaaccctcaggaaggcctgtataacgagctgcagaaggacaagatggccgaggcct |
| | | actccgaaattgggatgaagggagagcggcggaggggaaaggggcacgacggcctgtaccaag |
| | | gactgtccaccgccaccaaggacacatacgatgccctgcacatgcaggcccttccccctcgc |
| 28 | linker | (Gly-Gly-Gly-Ser)n, where n = 1-10 |
| 29 | linker | (Gly4 Ser)4 |
| 30 | linker | (Gly4 Ser)3 |
| 31 | linker | (Gly3Ser) |
| 39 | PD1 CAR | Pgwfldspdrpwnpptfspallvvtegdnatftcsfsntsesfvlnwyrmspsnqtdklaafpedr |
| | (aa) | sqpgqdcrfrvtqlpngrdfhmsvvrarrndsgtylcgaislapkaqikeslraelrvterraevpta |
| | | hpspsprpagqfqtlvtttpaprpptpaptiasqplslrpeacrpaaggavhtrgldfacdiyiwapl |
| | | agtcgvlllslvitlyckrgrkkllyifkqpfmrpvqttqeedgcscrfpeeeeggcelrvkfsrsada |
| | | paykqgqnqlynelnlgrreeydvldkrrgrdpemggkprrknpqeglynelqkdkmaeays |
| | | eigmkgerrrgkghdglyqglstatkdtydalhmqalppr |

In Vitro CAR-T Manufacture

While methods contemplated herein relate to in vivo transduction of cells, the challenges of in vitro manufacture are also appreciated.

In some embodiments, cells transduced the viral vector as described herein, are expanded, e.g., by a method described herein. In some embodiments, the cells are expanded in culture for a period of several hours (e.g., about 2, 3, 4, 5, 6, 7, 8, 9, 10, 15, 18, 21 hours) to about 14 days (e.g., 1, 2, 3, 4, 5, 6, 7, 8, 9, 10, 11, 12, 13 or 14 days). In some embodiments, the cells are expanded for a period of 4 to 9 days. In some embodiments, the cells are expanded for a period of 8 days or less, e.g., 7, 6 or 5 days. In some embodiments, the cells are expanded in culture for 5 days, and the resulting cells are more potent than the same cells expanded in culture for 9 days under the same culture conditions. Potency can be defined, e.g., by various T cell functions, e.g. proliferation, target cell killing, cytokine production, activation, migration, or combinations thereof. In some embodiments, the cells are expanded for 5 days show at least a one, two, three, or four-fold increase in cells doublings upon antigen stimulation as compared to the same cells expanded in culture for 9 days under the same culture conditions. In some embodiments, the cells are expanded in culture for 5 days, and the resulting cells exhibit higher proinflammatory cytokine production, e.g., IFN-7 and/or GM-CSF levels, as compared to the same cells expanded in culture for 9 days under the same culture conditions. In some embodiments, the cells expanded for 5 days show at least a one, two, three, four, five, ten-fold or more increase in μg/ml of proinflammatory cytokine production, e.g., IFN-7 and/or GM-CSF levels, as compared to the same cells expanded in culture for 9 days under the same culture conditions.

Initial activation steps in the absence of calcium can lead to magnified activation. As those of ordinary skill in the art would readily appreciate a washing step may be accomplished by methods known to those in the art, such as by using a semi-automated “flow-through” centrifuge (for example, the Cobe 2991 cell processor, the Baxter CytoMate, or the Haemonetics Cell Saver 5) according to the manufacturer's instructions. After washing, the cells may be resuspended in a variety of biocompatible buffers, such as, for example, Ca-free, Mg-free PBS, PlasmaLyte A, or other saline solution with or without buffer. Alternatively, the undesirable components of the apheresis sample may be removed, and the cells directly resuspended in culture media.

It is recognized that the in vitro methods of the application can utilize culture media conditions comprising 5% or less, for example 2%, human AB serum, and employ known culture media conditions and compositions, for example those described in Smith et al., “Ex vivo expansion of human T cells for adoptive immunotherapy using the novel Xeno-free CTS Immune Cell Serum Replacement” Clinical & Translational Immunology (2015) 4, e31; doi:10.1038/cti.2014.31.

In some aspects, T cells are isolated from peripheral blood lymphocytes by lysing the red blood cells and depleting the monocytes, for example, by centrifugation through a PERCOLL™ gradient or by counterflow centrifugal elutriation. The isolated T cells may be further used in the methods described herein.

The methods described herein can include, e.g., selection of a specific subpopulation of immune effector cells, e.g., T cells, that are a T regulatory cell-depleted population, CD25+ depleted cells, using, e.g., a negative selection technique, e.g., described herein. Preferably, the population of T regulatory depleted cells contains less than 30%, 25%, 20%, 15%, 10%, 5%, 4%, 3%, 2%, 1% of CD25+ cells.

In some embodiments, T regulatory cells, e.g., CD25+ T cells, are removed from the population using an anti-CD25 antibody, or fragment thereof, or a CD25-binding ligand, IL-2. In some embodiments, the anti-CD25 antibody, or fragment thereof, or CD25-binding ligand is conjugated to a substrate, e.g., a bead, or is otherwise coated on a substrate, e.g., a bead. In some embodiments, the anti-CD25 antibody, or fragment thereof, is conjugated to a substrate as described herein.

In some embodiments, the T regulatory cells, e.g., CD25+ T cells, are removed from the population using CD25 depletion reagent from Miltenyi™. In some embodiments, the ratio of cells to CD25 depletion reagent is 1×10⁷ cells to 20 μL, or 1×10⁷ cells to 15 μL, or 1×10⁷ cells to 10 μL, or 1×10⁷ cells to 5 μL, or 1×10⁷ cells to 2.5 μL, or 1×10⁷ cells to 1.25 μL. In some embodiments, e.g., for T regulatory cells, e.g., CD25+ depletion, greater than 500 million cells/ml is used. In a further aspect, a concentration of cells of 600, 700, 800, or 900 million cells/ml is used.

In some embodiments, the population of immune effector cells to be depleted includes about 6×10⁹ CD25+ T cells. In other aspects, the population of immune effector cells to be depleted include about 1×10⁹ to 1×10¹⁰ CD25+ T cell, and any integer value in between. In some embodiments, the resulting population T regulatory depleted cells has 2×10⁹ T regulatory cells, e.g., CD25+ cells, or less (e.g., 1×10⁹, 5×10⁸, 1×10⁸, 5×10⁷, 1×10⁷, or less CD25+ cells).

In some embodiments, the T regulatory cells, e.g., CD25+ cells, are removed from the population using the CliniMAC system with a depletion tubing set, such as, e.g., tubing 162-01. In some embodiments, the CliniMAC system is run on a depletion setting such as, e.g., DEPLETION2.1.

Without wishing to be bound by a particular theory, decreasing the level of negative regulators of immune cells (e.g., decreasing the number of unwanted immune cells, e.g., T_{REG}cells), in a subject prior to apheresis or during manufacturing of a CAR-expressing cell product can reduce the risk of subject relapse. For example, methods of depleting T_{REG} cells are known in the art. Methods of decreasing T_{REG} cells include, but are not limited to, cyclophosphamide, anti-GITR antibody (an anti-GITR antibody described herein), CD25-depletion, and combinations thereof.

In some embodiments, the manufacturing methods comprise reducing the number of (e.g., depleting) TREG cells prior to manufacturing of the CAR-expressing cell. For example, manufacturing methods comprise contacting the sample, e.g., the apheresis sample, with an anti-GITR antibody and/or an anti-CD25 antibody (or fragment thereof, or a CD25-binding ligand), e.g., to deplete T_{REG} cells prior to manufacturing of the CAR-expressing cell (e.g., T cell, NK cell) product.

In some embodiments, a subject is pre-treated with one or more therapies that reduce T_{REG} cells prior to collection of cells for CAR-expressing cell product manufacturing, thereby reducing the risk of subject relapse to CAR-expressing cell treatment. In some embodiments, methods of decreasing T_{REG} cells include, but are not limited to, administration to the subject of one or more of cyclophosphamide, anti-GITR antibody, CD25-depletion, or a combination thereof Administration of one or more of cyclophosphamide, anti-GITR antibody, CD25-depletion, or a combination thereof, can occur before, during or after an infusion of the CAR-expressing cell product.

In some embodiments, a subject is pre-treated with cyclophosphamide prior to collection of cells for CAR-expressing cell product manufacturing, thereby reducing the risk of subject relapse to CAR-expressing cell treatment. In some embodiments, a subject is pre-treated with an anti-GITR antibody prior to collection of cells for CAR-expressing cell product manufacturing, thereby reducing the risk of subject relapse to CAR-expressing cell treatment.

In some embodiments, the population of cells to be removed are neither the regulatory T cells or tumor cells, but cells that otherwise negatively affect the expansion and/or function of CART cells, e.g. cells expressing CD14, CD11b, CD33, CD15, or other markers expressed by potentially immune suppressive cells. In some embodiments, such cells are envisioned to be removed concurrently with regulatory T cells and/or tumor cells, or following said depletion, or in another order.

The methods described herein can include more than one selection step, e.g., more than one depletion step. Enrichment of a T cell population by negative selection can be accomplished, e.g., with a combination of antibodies directed to surface markers unique to the negatively selected cells. One method is cell sorting and/or selection via negative magnetic immunoadherence or flow cytometry that uses a cocktail of monoclonal antibodies directed to cell surface markers present on the cells negatively selected. For example, to enrich for CD4+ cells by negative selection, a monoclonal antibody cocktail can include antibodies to CD14, CD20, CD11b, CD16, HLA-DR, and CD8.

The methods described herein can further include removing cells from the population which express a tumor antigen, e.g., a tumor antigen that does not comprise CD25, e.g., CD19, CD30, CD38, CD123, CD20, CD14 or CD11b, to thereby provide a population of T regulatory depleted, e.g., CD25+ depleted, and tumor antigen depleted cells that are suitable for expression of a CAR, e.g., a CAR described herein. In some embodiments, tumor antigen expressing cells are removed simultaneously with the T regulatory, e.g., CD25+ cells. For example, an anti-CD25 antibody, or fragment thereof, and an anti-tumor antigen antibody, or fragment thereof, can be attached to the same substrate, e.g., bead, which can be used to remove the cells or an anti-CD25 antibody, or fragment thereof, or the anti-tumor antigen antibody, or fragment thereof, can be attached to separate beads, a mixture of which can be used to remove the cells. In other embodiments, the removal of T regulatory cells, e.g., CD25+ cells, and the removal of the tumor antigen expressing cells is sequential, and can occur, e.g., in either order.

Also provided are methods that include removing cells from the population which express a check point inhibitor, e.g., a check point inhibitor described herein, e.g., one or more of PD1+ cells, LAG3+ cells, and TIM3+ cells, to thereby provide a population of T regulatory depleted, e.g., CD25+ depleted cells, and check point inhibitor depleted cells, e.g., PD1+, LAG3+ and/or TIM3+ depleted cells. Exemplary check point inhibitors include B7-H1, B7-1, CD160, P1H, 2B4, PD1, TIM3, CEACAM (e.g., CEACAM-1, CEACAM-3 and/or CEACAM-5), LAG3, TIGIT, CTLA-4, BTLA, and LAIR1. In some embodiments, check point inhibitor expressing cells are removed simultaneously with the T regulatory, e.g., CD25+ cells. For example, an anti-CD25 antibody, or fragment thereof, and an anti-check point inhibitor antibody, or fragment thereof, can be attached to the same bead which can be used to remove the cells, or an anti-CD25 antibody, or fragment thereof, and the anti-check point inhibitor antibody, or fragment there, can be attached to separate beads, a mixture of which can be used to remove the cells. In other embodiments, the removal of T regulatory cells, e.g., CD25+ cells, and the removal of the check point inhibitor expressing cells is sequential, and can occur, e.g., in either order.

Methods described herein can include a positive selection step. For example, T cells can isolated by incubation with anti-CD3/anti-CD28 (e.g., 3×28)-conjugated beads, such as DYNABEADS® M-450 CD3/CD28 T, for a time period sufficient for positive selection of the desired T cells. In some embodiments, the time period is about 30 minutes. In a further embodiment, the time period ranges from 30 minutes to 36 hours or longer and all integer values there between. In a further embodiment, the time period is at least 1, 2, 3, 4, 5, or 6 hours. In yet another embodiment, the time period is 10 to 24 hours, e.g., 24 hours. Longer incubation times may be used to isolate T cells in any situation where there are few T cells as compared to other cell types, such in isolating tumor infiltrating lymphocytes (TIL) from tumor tissue or from immunocompromised individuals. Further, use of longer incubation times can increase the efficiency of capture of CD8+ T cells. Thus, by simply shortening or lengthening the time T cells are allowed to bind to the CD3/CD28 beads and/or by increasing or decreasing the ratio of beads to T cells (as described further herein), subpopulations of T cells can be preferentially selected for or against at culture initiation or at other time points during the process. Additionally, by increasing or decreasing the ratio of anti-CD3 and/or anti-CD28 antibodies on the beads or other surface, subpopulations of T cells can be preferentially selected for or against at culture initiation or at other desired time points. In some embodiments, a T cell population can be selected that expresses one or more of IFN-γ, TNFα, IL-17A, IL-2, IL-3, IL-4, GM-CSF, IL-10, IL-13, granzyme B, and perforin, or other appropriate molecules, e.g., other cytokines. Methods for screening for cell expression can be determined, e.g., by the methods described in PCT Publication No.: WO 2013/126712.

For isolation of a desired population of cells by positive or negative selection, the concentration of cells and surface (e.g., particles such as beads) can be varied. In some aspects, it may be desirable to significantly decrease the volume in which beads and cells are mixed together (e.g., increase the concentration of cells), to ensure maximum contact of cells and beads. For example, in some aspects, a concentration of 10 billion cells/ml, 9 billion/ml, 8 billion/ml, 7 billion/ml, 6 billion/ml, or 5 billion/ml is used. In some aspects, a concentration of 1 billion cells/ml is used. In yet some aspects, a concentration of cells from 75, 80, 85, 90, 95, or 100 million cells/ml is used. In further aspects, concentrations of 125 or 150 million cells/ml can be used.

Using high concentrations can result in increased cell yield, cell activation, and cell expansion. Further, use of high cell concentrations allows more efficient capture of cells that may weakly express target antigens of interest, such as CD28-negative T cells, or from samples where there are many tumor cells present (e.g., leukemic blood, tumor tissue, etc.). Such populations of cells may have therapeutic value and would be desirable to obtain. For example, using high concentration of cells allows more efficient selection of CD8+ T cells that normally have weaker CD28 expression.

In some embodiments, it may be desirable to use lower concentrations of cells. By significantly diluting the mixture of T cells and surface (e.g., particles such as beads), interactions between the particles and cells are minimized. This selects for cells that express high amounts of desired antigens to be bound to the particles. For example, CD4+ T cells express higher levels of CD28 and are more efficiently captured than CD8+ T cells in dilute concentrations. In some aspects, the concentration of cells used is 5×10⁶/ml. In other aspects, the concentration used can be from about 1×10⁵/ml to 1×10⁶/ml, and any integer value in between.

In other aspects, the cells may be incubated on a rotator for varying lengths of time at varying speeds at either 2-10° C. or at room temperature.

T cells for stimulation can also be frozen after a washing step. Wishing not to be bound by theory, the freeze and subsequent thaw step provide a more uniform product by removing granulocytes and to some extent monocytes in the cell population. After the washing step that removes plasma and platelets, the cells may be suspended in a freezing solution. While many freezing solutions and parameters are known in the art and will be useful in this context, one method involves using PBS containing 20% DMSO and 8% human serum albumin, or culture media containing 10% Dextran 40 and 5% Dextrose, 20% Human Serum Albumin and 7.5% DMSO, or 31.25% Plasmalyte-A, 31.25% Dextrose 5%, 0.45% NaCl, 10% Dextran 40 and 5% Dextrose, 20% Human Serum Albumin, and 7.5% DMSO or other suitable cell freezing media containing for example, Hespan and PlasmaLyte A, the cells then are frozen to −80° C. at a rate of 1° per minute and stored in the vapor phase of a liquid nitrogen storage tank. Other methods of controlled freezing may be used as well as uncontrolled freezing immediately at −20° C. or in liquid nitrogen.

In some aspects, cryopreserved cells are thawed and washed as described herein and allowed to rest for one hour at room temperature prior to activation using the methods of the present invention.

Also contemplated in the context of the invention is the collection of blood samples or apheresis product from a subject at a time period prior to when the expanded cells as described herein might be needed. As such, the source of the cells to be expanded can be collected at any time point necessary, and desired cells, such as T cells, can be isolated and frozen for later use in immune effector cell therapy for any number of diseases or conditions that would benefit from immune effector cell therapy, such as those described herein. In some aspects a blood sample or an apheresis is taken from a generally healthy subject. In some aspects, a blood sample or an apheresis is taken from a generally healthy subject who is at risk of developing a disease, but who has not yet developed a disease, and the cells of interest are isolated and frozen for later use. In some aspects, the T cells may be expanded, frozen, and used at a later time. In some aspects, samples are collected from a patient shortly after diagnosis of a particular disease as described herein but prior to any treatments. In a further aspect, the cells are isolated from a blood sample or an apheresis from a subject prior to any number of relevant treatment modalities, including but not limited to treatment with agents such as natalizumab, efalizumab, antiviral agents, chemotherapy, radiation, immunosuppressive agents, such as cyclosporin, azathioprine, methotrexate, mycophenolate, and FK506, antibodies, or other immunoablative agents such as CAMPATH, anti-CD3 antibodies, cytoxan, fludarabine, cyclosporin, FK506, rapamycin, mycophenolic acid, steroids, FR901228, and irradiation.

In a further aspect of the present invention, T cells are obtained from a patient directly following treatment that leaves the subject with functional T cells. In this regard, it has been observed that following certain cancer treatments, in particular treatments with drugs that damage the immune system, shortly after treatment during the period when patients would normally be recovering from the treatment, the quality of T cells obtained may be optimal or improved for their ability to expand ex vivo. Likewise, following ex vivo manipulation using the methods described herein, these cells may be in a preferred state for enhanced engraftment and in vivo expansion. Thus, it is contemplated within the context of the present invention to collect blood cells, including T cells, dendritic cells, or other cells of the hematopoietic lineage, during this recovery phase. Further, in some aspects, mobilization (for example, mobilization with GM-CSF) and conditioning regimens can be used to create a condition in a subject wherein repopulation, recirculation, regeneration, and/or expansion of particular cell types is favored, especially during a defined window of time following therapy. Illustrative cell types include T cells, B cells, dendritic cells, and other cells of the immune system.

In some embodiments, a T cell population is diaglycerol kinase (DGK)-deficient. DGK-deficient cells include cells that do not express DGK RNA or protein or have reduced or inhibited DGK activity. DGK-deficient cells can be generated by genetic approaches, e.g., administering RNA-interfering agents, e.g., siRNA, shRNA, miRNA, to reduce or prevent DGK expression. Alternatively, DGK-deficient cells can be generated by treatment with DGK inhibitors described herein.

In some embodiments, a T cell population is Ikaros-deficient. Ikaros-deficient cells include cells that do not express Ikaros RNA or protein, or have reduced or inhibited Ikaros activity, Ikaros-deficient cells can be generated by genetic approaches, e.g., administering RNA-interfering agents, e.g., siRNA, shRNA, miRNA, to reduce or prevent Ikaros expression. Alternatively, Ikaros-deficient cells can be generated by treatment with Ikaros inhibitors, e.g., lenalidomide.

In embodiments, a T cell population is DGK-deficient and Ikaros-deficient, e.g., does not express DGK and Ikaros, or has reduced or inhibited DGK and Ikaros activity. Such DGK and Ikaros-deficient cells can be generated by any of the methods described herein.

In some embodiments, the NK cells are obtained from the subject. In another embodiment, the NK cells are an NK cell line, e.g., NK-92 cell line (Conkwest).

In a particular exemplary aspect, subjects may undergo leukapheresis, wherein leukocytes are collected, enriched, or depleted ex vivo to select and/or isolate the cells of interest, e.g., T cells. These T cell isolates may be expanded by methods described herein. Subjects in need thereof may subsequently undergo standard treatment with high dose chemotherapy followed by peripheral blood stem cell transplantation. In some aspects, following or concurrent with the transplant, subjects receive an infusion of the expanded CAR T cells as prepared by the methods of the present invention. In an additional aspect, expanded cells are administered before or following surgery.

Additional Expressed Agents

Co-Expression of an Agent that Enhances CAR Activity

In the embodiments contemplated herein, it is appreciated that additional agents may be encoded in the vectors described herein above. Accordingly, these agents are described below in relation to the CAR-expressing cell.

In another embodiment, a CAR-expressing immune effector cell described herein can further express another agent, e.g., an agent which enhances the activity of a CAR-expressing cell. For example, in some embodiments, the agent can be an agent which inhibits an inhibitory molecule. Examples of inhibitory molecules include PD-1, PD-L1, CTLA-4, TIM-3, CEACAM (e.g., CEACAM-1, CEACAM-3 and/or CEACAM-5), LAG-3, VISTA, BTLA, TIGIT, LAIR1, CD160, 2B4 and TGFR beta, e.g., as described herein. In some embodiments, the agent that inhibits an inhibitory molecule comprises a first polypeptide, e.g., an inhibitory molecule, associated with a second polypeptide that provides a positive signal to the cell, e.g., an intracellular signaling domain described herein. In some embodiments, the agent comprises a first polypeptide, e.g., of an inhibitory molecule such as PD-1, PD-L1, CTLA-4, TIM-3, CEACAM (e.g., CEACAM-1, CEACAM-3 and/or CEACAM-5), LAG-3, VISTA, BTLA, TIGIT, LAIR1, CD160, 2B4, or TGFR beta, or a fragment of any of these, and a second polypeptide which is an intracellular signaling domain described herein (e.g., comprising a costimulatory domain (e.g., 41BB, CD27 or CD28, e.g., as described herein) and/or a primary signaling domain (e.g., a CD3 zeta signaling domain described herein). In some embodiments, the agent comprises a first polypeptide of PD-1 or a fragment thereof, and a second polypeptide of an intracellular signaling domain described herein (e.g., a CD28, CD27, OX40 or 4-IBB signaling domain described herein and/or a CD3 zeta signaling domain described herein).

Co-Expression of a Second CAR

In some embodiments, the CAR-expressing cell described herein can further comprise a second CAR, for example, a second CAR that includes a different antigen binding domain, for example, to the same target (for example, CD19) or a different target (for example, a target other than CD19, for example, a target described herein).

In some embodiments, the CAR-expressing cell described herein, e.g., the CAR-expressing cell manufactured using a method described herein, comprises (i) a first nucleic acid molecule encoding a first CAR that binds to BCMA and (ii) a second nucleic acid molecule encoding a second CAR that binds to CD19. In some embodiments, the first CAR comprises an anti-BCMA binding domain, a first transmembrane domain, and a first intracellular signaling domain, wherein the anti-BCMA binding domain comprises a heavy chain variable region (VH) comprising a heavy chain complementary determining region 1 (HC CDR1), a heavy chain complementary determining region 2 (HC CDR2), and a heavy chain complementary determining region 3 (HC CDR3), and a light chain variable region (VL) comprising a light chain complementary determining region 1 (LC CDR1), a light chain complementary determining region 2 (LC CDR2), and a light chain complementary determining region 3 (LC CDR3), wherein the HC CDR1, HC CDR2, HC CDR3, LC CDR1, LC CDR2, and LC CDR3 comprise the amino acid sequences of SEQ ID NOs: 86, 87, 88, 95, 96, and 97, respectively. In some embodiments, the second CAR comprises an anti-CD19 binding domain, a second transmembrane domain, and a second intracellular signaling domain, wherein the anti-CD19 binding domain comprises a VH comprising a HC CDR1, a HC CDR2, and a HC CDR3, and a VL comprising a LC CDR1, a LC CDR2, and a LC CDR3, wherein the HC CDR1, HC CDR2, HC CDR3, LC CDR1, LC CDR2, and LC CDR3 comprise the amino acid sequences of SEQ ID NOs: 760, 687, 762, 763, 764, and 765, respectively. In some embodiments, (i) the VH and VL of the anti-BCMA binding domain comprise the amino acid sequences of SEQ ID NOs: 93 and 102, respectively. In some embodiments, the VH and VL of the anti-CD19 binding domain comprise the amino acid sequences of SEQ ID NOs: 250A and 251A, respectively. In some embodiments, the anti-BCMA binding domain comprises the amino acid sequence of SEQ ID NO: 105. In some embodiments, the anti-CD19 binding domain comprises the amino acid sequence of SEQ ID NO: 758. In some embodiments, the first CAR comprises the amino acid sequence of SEQ ID NO: 107. In some embodiments, the second CAR comprise the amino acid sequence of SEQ ID NO: 225.

In some embodiments, the CAR-expressing cell described herein, e.g., the CAR-expressing cell manufactured using a method described herein, comprises (i) a first nucleic acid molecule encoding a first CAR that binds to CD22 and (ii) a second nucleic acid molecule encoding a second CAR that binds to CD19. In some embodiments, the CD22 CAR comprises a CD22 antigen binding domain, and a first transmembrane domain; a first co-stimulatory signaling domain; and/or a first primary signaling domain. In some embodiments, the CD19 CAR comprises a CD19 antigen binding domain, and a second transmembrane domain; a second co-stimulatory signaling domain; and/or a second primary signaling domain.

In some embodiments, the CD22 antigen binding domain comprises one or more (e.g., all three) light chain complementarity determining region 1 (LC CDR1), light chain complementarity determining region 2 (LC CDR2), and light chain complementarity determining region 3 (LC CDR3) of a CD22 binding domain described herein, e.g., in Tables 15, 16, 30, 31, or 32; and/or one or more (e.g., all three) heavy chain complementarity determining region 1 (HC CDR1), heavy chain complementarity determining region 2 (HC CDR2), and heavy chain complementarity determining region 3 (HC CDR3) of a CD22 binding domain described herein, e.g., in Tables 15, 16, 30, 31 or 32. In an embodiment, the CD22 antigen binding domain comprises a LC CDR1, LC CDR2 and LC CDR3 of a CD22 binding domain described herein, e.g., in Table 15, 16, 30, 31 or 32; and/or a HC CDR1, HC CDR2 and HC CDR3 of a CD22 binding domain described herein, e.g., in Tables 15, 16, 30, 31 or 32. In some embodiments, the CD19 antigen binding domain comprises: one or more (e.g., all three) LC CDR1, LC CDR2, and LC CDR3 of a CD19 binding domain described herein, e.g., in Tables 1, 30, 31, or 32; and/or one or more (e.g., all three) HC CDR1, HC CDR2, and HC CDR3 of a CD19 binding domain described herein, e.g., in Tables 1, 30, 31, and 32. In some embodiments, the CD19 antigen binding domain comprises a LC CDR1, LC CDR2 and LC CDR3 of a CD19 binding domain described herein, e.g., in Tables 1, 30, 31, and 32; and/or a HC CDR1, HC CDR2 and HC CDR3 of a CD19 binding domain described herein, e.g., in Tables 1, 30, 31, and 32.

In some embodiment, the CD22 antigen binding domain (e.g., an scFv) comprises a light chain variable (VL) region of a CD22 binding domain described herein, e.g., in Tables 30 or 32; and/or a heavy chain variable (VH) region of a CD22 binding domain described herein, e.g., in Tables 30 or 32. In some embodiments, the CD22 antigen binding domain comprises a VL region comprising an amino acid sequence having at least one, two or three modifications (e.g., substitutions) but not more than 30, 20 or 10 modifications (e.g., substitutions) of a CD22 VL region sequence provided in Table 30 or 32. In some embodiments, the CD22 antigen binding domain comprises a VL region comprising an amino acid sequence provided in Table 30 or 32, or a sequence having at least about 80%, 85%, 90%, 92%, 95%, 97%, 98%, or 99% sequence identity to any of the aforesaid sequences. In some embodiments, the CD22 antigen binding domain comprises a VH region comprising an amino acid sequence having at least one, two or three modifications (e.g., substitutions) but not more than 30, 20 or 10 modifications (e.g., substitutions) of a CD22 VH region sequence provided in Table 30 or 32. In some embodiments, the CD22 antigen binding domain comprises a VH region comprising the amino acid sequence of a CD22 VH region sequence provided in Table 30 or 32, or a sequence having at least about 80%, 85%, 90%, 92%, 95%, 97%, 98%, or 99% sequence identity to any of the aforesaid sequences. In some embodiments, the CD19 antigen binding domain (e.g., an scFv) comprises a VL region of a CD19 binding domain described herein, e.g., in Tables 1, 30, or 32; and/or a VH region of a CD19 binding domain described herein, e.g., in Tables 1, 30, or 32. In some embodiments, the CD19 antigen binding domain comprises a VL region comprising an amino acid sequence having at least one, two or three modifications (e.g., substitutions) but not more than 30, 20 or 10 modifications (e.g., substitutions) of a CD19 VL region sequence provided in Tables 1, 30, or 32. In some embodiments, the CD19 antigen binding domain comprises a VL region comprising the amino acid sequence of a CD19 VL region sequence provided in Tables 1, 30, or 32, or a sequence having at least about 80%, 85%, 90%, 92%, 95%, 97%, 98%, or 99% sequence identity to any of the aforesaid sequences. In some embodiments, the CD19 antigen binding domain comprises a VH region comprising an amino acid sequence having at least one, two or three modifications (e.g., substitutions) but not more than 30, 20 or 10 modifications (e.g., substitutions) of a CD19 VH region sequence provided in Tables 1, 30, or 32. In some embodiments, the CD19 antigen binding domain comprises a VH region comprising the amino acid sequence of a CD19 VH region sequence provided in Tables 1, 30, or 32, or a sequence having at least about 80%, 85%, 90%, 92%, 95%, 97%, 98%, or 99% sequence identity to any of the aforesaid sequences.

In some embodiments, the CD22 antigen binding comprises an scFv comprising an amino acid sequence having at least one, two or three modifications (e.g., substitutions) but not more than 30, 20 or 10 modifications (e.g., substitutions) of a CD22 scFv sequence provided in Table 30 or 32. In some embodiments, the CD22 antigen binding comprises an scFv comprising an amino acid sequence of a CD22 scFv sequence provided in Table 30 or 32, or a sequence having at least about 80%, 85%, 90%, 92%, 95%, 97%, 98%, or 99% sequence identity to any of the aforesaid sequences. In some embodiments, the CD19 antigen binding domain comprises an scFv comprising an amino acid sequence having at least one, two or three modifications (e.g., substitutions) but not more than 30, 20 or 10 modifications (e.g., substitutions) of a CD19 scFv sequence provided in Tables 1, 30, or 32. In some embodiments, the CD19 antigen binding domain comprises an scFv comprising the amino acid sequence of a CD19 scFv sequence provided in Tables 1, 30, or 32, or a sequence having at least about 80%, 85%, 90%, 92%, 95%, 97%, 98%, or 99% sequence identity to any of the aforesaid sequences.

In some embodiments, the CD22 CAR molecule and/or the CD19 CAR molecule comprises an additional component, e.g., a signal peptide, a hinge, a transmembrane domain, a co-stimulatory signaling domain and/or a first primary signaling domain, a P2A site, and/or a linker, comprising an amino acid sequence provided in Table 33, or a sequence having at least about 70%, 75%, 80%, 85%, 90%, 92%, 95%, 97%, 98%, or 99% sequence identity to any of the aforesaid sequences; or is encoded by a nucleotide sequence provided in Table 33, or a sequence having at least about 70%, 75%, 80%, 85%, 90%, 92%, 95%, 97%, 98%, or 99% sequence identity to any of the aforesaid sequences.

Exemplary nucleotide and amino acid sequences of a CAR molecule, e.g., a dual CAR molecule comprising (i) a first CAR that binds to CD22 and (ii) a second CAR that binds to CD19 disclosed herein, is provided in Table 30.

TABLE 30
Dual and tandem CD19-CD22 CAR sequences

| | | |
|---|---|---|
| | SEQ ID | |
| Identifier | NO | Sequence |

Tandem CD19-CD22 CARS

| | | |
|---|---|---|
| CG#c171 | 813 | atggccctccctgtcaccgccctgctgcttccgctggctcttctgctccacgccgctcggcccg |
| | | aaattgtgatgacccagtcacccgccactcttagcctttcacccggtgagcgcgcaaccctgtc |
| | | ttgcagagcctcccaagacatctcaaaataccttaattggtatcaacagaagcccggacaggc |
| | | tcctcgccttctgatctaccacaccagccggctccattctggaatccctgccaggttcagcggt |
| | | agcggatctgggaccgactacaccctcactatcagctcactgcagccagaggacttcgctgtc |
| | | tatttctgtcagcaagggaacaccctgccctacacctttggacagggcaccaagctcgagatt |
| | | aaaggtggaggtggcagcggaggaggtgggtccggcggtggaggaagccaggtccaact |
| | | ccaagaaagcggaccgggtcttgtgaagccatcagaaactctttcactgacttgtactgtgagc |
| | | ggagtgtctctccccgattacggggtgtcttggatcagacagccaccggggaagggtctgga |
| | | atggattggagtgatttggggctctgagactacttactaccaatcatccctcaagtcacgcgtca |
| | | ccatctcaaaggacaactctaagaatcaggtgtcactgaaactgtcatctgtgaccgcagccg |
| | | acaccgccgtgtactattgcgctaagcattactattatggcgggagctacgcaatggattactg |
| | | gggacagggtactctggtcaccgtgtccagcttggcagaagccgccgcgaaagaagtgcag |
| | | cttcaacaatcaggaccaggactcgtcaaaccatcacagaccctctccctcacatgtgccatct |
| | | ccggggactccatgttgagcaattccgacacttggaattggattagacaaagcccgtcccggg |
| | | gtctggaatggttgggacgcacctaccaccggtctacttggtacgacgactacgcgtcatccg |
| | | tgcggggaagagtgtccatcaacgtggacacctccaagaaccagtacagcctgcagcttaat |
| | | gccgtgactcctgaggatacgggcgtctactactgcgcccgcgtccgcctgcaagacggga |
| | | acagctggagcgatgcattcgatgtctggggccagggaactatggtcaccgtgtcgtctggg |
| | | ggcggtggatcgggtggcgggggttcggggggggcggctctcagtccgctcttacccaac |
| | | cggcctcagcctcggggagccccggccagagcgtgaccatttcctgcaccggcacttcatcc |
| | | gacgtgggcggctacaactacgtgtcctggtaccaacagcacccgggaaaggcccccaag |
| | | ctcatgatctacgacgtgtccaacaggccctcgggagtgtccaaccggttctcgggttcgaaa |
| | | tcgggaaacacagccagcctgaccatcagcggactgcaggctgaagatgaagccgactact |
| | | actgctcctcctacacctcgtcatccacgctctacgtgttcggcactggaactcagctgactgtg |
| | | ctgaccactaccccagcaccgaggccacccaccccggctcctaccatcgcctcccagcctct |
| | | gtccctgcgtccggaggcatgtagacccgcagctggtggggccgtgcatacccggggtctt |
| | | gacttcgcctgcgatatctacatttgggcccctctggctggtacttgcggggtcctgctgctttca |
| | | ctcgtgatcactctttactgtaagcgcggtcggaagaagctgctgtacatctttaagcaaccctt |
| | | catgaggcctgtgcagactactcaagaggaggacggctgttcatgccggttcccagaggag |
| | | gaggaaggcggctgcgaactgcgcgtgaaattcagccgcagcgcagatgctccagcctac |
| | | cagcaggggcagaaccagctctacaacgaactcaatcttggtcggagagaggagtacgacg |
| | | tgctggacaagcggagaggacgggacccagaaatgggcgggaagccgcgcagaaagaa |
| | | tccccaagagggcctgtacaacgagctccaaaaggataagatggcagaagcctatagcgag |
| | | attggtatgaaaggggaacgcagaagaggcaaaggccacgacggactgtaccagggactc |
| | | agcaccgccaccaaggacacctatgacgctcttcacatgcaggccctgccgcctcgg |
| | 814 | MALPVTALLLPLALLLHAARPEIVMTQSPATLSLSPGERAT |
| | | LSCRASQDISKYLNWYQQKPGQAPRLLIYHTSRLHSGIPAR |
| | | FSGSGSGTDYTLTISSLQPEDFAVYFCQQGNTLPYTFGQGT |
| | | KLEIKGGGGSGGGGSGGGGSQVQLQESGPGLVKPSETLSLT |
| | | CTVSGVSLPDYGVSWIRQPPGKGLEWIGVIWGSETTYYQSS |
| | | LKSRVTISKDNSKNQVSLKLSSVTAADTAVYYCAKHYYYG |
| | | GSYAMDYWGQGTLVTVSSLAEAAAKEVQLQQSGPGLVKP |
| | | SQTLSLTCAISGDSMLSNSDTWNWIRQSPSRGLEWLGRTY |
| | | HRSTWYDDYASSVRGRVSINVDTSKNQYSLQLNAVTPEDT |
| | | GVYYCARVRLQDGNSWSDAFDVWGQGTMVTVSSGGGGS |
| | | GGGGSGGGGSQSALTQPASASGSPGQSVTISCTGTSSDVGG |
| | | YNYVSWYQQHPGKAPKLMIYDVSNRPSGVSNRFSGSKSG |
| | | NTASLTISGLQAEDEADYYCSSYTSSSTLYVFGTGTQLTVL |
| | | TTTPAPRPPTPAPTIASQPLSLRPEACRPAAGGAVHTRGLDF |
| | | ACDIYIWAPLAGTCGVLLLSLVITLYCKRGRKKLLYIFKQPF |
| | | MRPVQTTQEEDGCSCRFPEEEEGGCELRVKFSRSADAPAY |
| | | QQGQNQLYNELNLGRREEYDVLDKRRGRDPEMGGKPRRK |
| | | NPQEGLYNELQKDKMAEAYSEIGMKGERRRGKGHDGLYQ |
| | | GLSTATKDTYDALHMQALPPR |
| CG#c182 | 815 | atggccctccctgtcaccgccctgctgcttccgctggctcttctgctccacgccgctcggcccg |
| | | aagtgcagcttcaacaatcaggaccaggactcgtcaaaccatcacagaccctctccctcacat |
| | | gtgccatctccggggactccatgttgagcaattccgacacttggaattggattagacaaagccc |
| | | gtcccggggtctggaatggttgggacgcacctaccaccggtctacttggtacgacgactacg |
| | | cgtcatccgtgcggggaagagtgtccatcaacgtggacacctccaagaaccagtacagcctg |
| | | cagcttaatgccgtgactcctgaggatacgggcgtctactactgcgcccgcgtccgcctgcaa |
| | | gacgggaacagctggagcgatgcattcgatgtctggggccagggaactatggtcaccgtgtc |
| | | gtctgggggcggtggatcgggtggcgggggttcggggggggcggctctcagtccgctctt |
| | | acccaaccggcctcagcctcggggagccccggccagagcgtgaccatttcctgcaccggc |
| | | acttcatccgacgtgggcggctacaactacgtgtcctggtaccaacagcacccgggaaaggc |
| | | ccccaagctcatgatctacgacgtgtccaacaggccctcgggagtgtccaaccggttctcgg |
| | | gttcgaaatcgggaaacacagccagcctgaccatcagcggactgcaggctgaagatgaagc |
| | | cgactactactgctcctcctacacctcgtcatccacgctctacgtgttcggcactggaactcagc |
| | | tgactgtgctgggagggggagggagtgaaattgtgatgacccagtcacccgccactcttagc |
| | | ctttcacccggtgagcgcgcaaccctgtcttgcagagcctcccaagacatctcaaaatacctta |
| | | attggtatcaacagaagcccggacaggctcctcgccttctgatctaccacaccagccggctcc |
| | | attctggaatccctgccaggttcagcggtagcggatctgggaccgactacaccctcactatca |
| | | gctcactgcagccagaggacttcgctgtctatttctgtcagcaagggaacaccctgccctaca |
| | | cctttggacagggcaccaagctcgagattaaaggtggaggtggcagcggaggaggtgggt |
| | | ccggcggtggaggaagccaggtccaactccaagaaagcggaccgggtcttgtgaagccat |
| | | cagaaactctttcactgacttgtactgtgagcggagtgtctctccccgattacggggtgtcttgg |
| | | atcagacagccaccggggaagggtctggaatggattggagtgatttggggctctgagactac |
| | | ttactaccaatcatccctcaagtcacgcgtcaccatctcaaaggacaactctaagaatcaggtg |
| | | tcactgaaactgtcatctgtgaccgcagccgacaccgccgtgtactattgcgctaagcattact |
| | | attatggcgggagctacgcaatggattactggggacagggtactctggtcaccgtgtccagca |
| | | ccactaccccagcaccgaggccacccaccccggctcctaccatcgcctcccagcctctgtcc |
| | | ctgcgtccggaggcatgtagacccgcagctggtggggccgtgcatacccggggtcttgactt |
| | | cgcctgcgatatctacatttgggcccctctggctggtacttgcggggtcctgctgctttcactcgt |
| | | gatcactctttactgtaagcgcggtcggaagaagctgctgtacatctttaagcaacccttcatga |
| | | ggcctgtgcagactactcaagaggaggacggctgttcatgccggttcccagaggaggagga |
| | | aggcggctgcgaactgcgcgtgaaattcagccgcagcgcagatgctccagcctaccagcag |
| | | gggcagaaccagctctacaacgaactcaatcttggtcggagagaggagtacgacgtgctgg |
| | | acaagcggagaggacgggacccagaaatgggcgggaagccgcgcagaaagaatcccca |
| | | agagggcctgtacaacgagctccaaaaggataagatggcagaagcctatagcgagattggt |
| | | atgaaaggggaacgcagaagaggcaaaggccacgacggactgtaccagggactcagcac |
| | | cgccaccaaggacacctatgacgctcttcacatgcaggccctgccgcctcgg |
| | 816 | MALPVTALLLPLALLLHAARPEVQLQQSGPGLVKPSQTLSL |
| | | TCAISGDSMLSNSDTWNWIRQSPSRGLEWLGRTYHRSTWY |
| | | DDYASSVRGRVSINVDTSKNQYSLQLNAVTPEDTGVYYCA |
| | | RVRLQDGNSWSDAFDVWGQGTMVTVSSGGGGSGGGGSG |
| | | GGGSQSALTQPASASGSPGQSVTISCTGTSSDVGGYNYVS |
| | | WYQQHPGKAPKLMIYDVSNRPSGVSNRFSGSKSGNTASLT |
| | | ISGLQAEDEADYYCSSYTSSSTLYVFGTGTQLTVLGGGGSE |
| | | IVMTQSPATLSLSPGERATLSCRASQDISKYLNWYQQKPGQ |
| | | APRLLIYHTSRLHSGIPARFSGSGSGTDYTLTISSLQPEDFAV |
| | | YFCQQGNTLPYTFGQGTKLEIKGGGGSGGGGSGGGGSQVQ |
| | | LQESGPGLVKPSETLSLTCTVSGVSLPDYGVSWIRQPPGKG |
| | | LEWIGVIWGSETTYYQSSLKSRVTISKDNSKNQVSLKLSSV |
| | | TAADTAVYYCAKHYYYGGSYAMDYWGQGTLVTVSSTTT |
| | | PAPRPPTPAPTIASQPLSLRPEACRPAAGGAVHTRGLDFAC |
| | | DIYIWAPLAGTCGVLLLSLVITLYCKRGRKKLLYIFKQPFM |
| | | RPVQTTQEEDGCSCRFPEEEEGGCELRVKFSRSADAPAYQQ |
| | | GQNQLYNELNLGRREEYDVLDKRRGRDPEMGGKPRRKNP |
| | | QEGLYNELQKDKMAEAYSEIGMKGERRRGKGHDGLYQGL |
| | | STATKDTYDALHMQALPPR |
| CG#c188 | 817 | atggccctccctgtcaccgccctgctgcttccgctggctcttctgctccacgccgctcggcccc |
| | | agtccgctcttacccaaccggcctcagcctcggggagccccggccagagcgtgaccatttcc |
| | | tgcaccggcacttcatccgacgtgggcggctacaactacgtgtcctggtaccaacagcaccc |
| | | gggaaaggcccccaagctcatgatctacgacgtgtccaacaggccctcgggagtgtccaac |
| | | cggttctcgggttcgaaatcgggaaacacagccagcctgaccatcagcggactgcaggctg |
| | | aagatgaagccgactactactgctcctcctacacctcgtcatccacgctctacgtgttcggcact |
| | | ggaactcagctgactgtgctgggcggaggaggctccgaagtgcagcttcaacaatcaggac |
| | | caggactcgtcaaaccatcacagaccctctccctcacatgtgccatctccggggactccatgtt |
| | | gagcaattccgacacttggaattggattagacaaagcccgtcccggggtctggaatggttggg |
| | | acgcacctaccaccggtctacttggtacgacgactacgcgtcatccgtgcggggaagagtgt |
| | | ccatcaacgtggacacctccaagaaccagtacagcctgcagcttaatgccgtgactcctgag |
| | | gatacgggcgtctactactgcgcccgcgtccgcctgcaagacgggaacagctggagcgatg |
| | | cattcgatgtctggggccagggaactatggtcaccgtgtcgtctggagggggagggagtgaa |
| | | attgtgatgacccagtcacccgccactcttagcctttcacccggtgagcgcgcaaccctgtctt |
| | | gcagagcctcccaagacatctcaaaataccttaattggtatcaacagaagcccggacaggctc |
| | | ctcgccttctgatctaccacaccagccggctccattctggaatccctgccaggttcagcggtag |
| | | cggatctgggaccgactacaccctcactatcagctcactgcagccagaggacttcgctgtcta |
| | | tttctgtcagcaagggaacaccctgccctacacctttggacagggcaccaagctcgagattaa |
| | | aggtggaggtggcagcggaggaggtgggtccggcggtggaggaagccaggtccaactcc |
| | | aagaaagcggaccgggtcttgtgaagccatcagaaactctttcactgacttgtactgtgagcg |
| | | gagtgtctctccccgattacggggtgtcttggatcagacagccaccggggaagggtctggaa |
| | | tggattggagtgatttggggctctgagactacttactaccaatcatccctcaagtcacgcgtcac |
| | | catctcaaaggacaactctaagaatcaggtgtcactgaaactgtcatctgtgaccgcagccga |
| | | caccgccgtgtactattgcgctaagcattactattatggcgggagctacgcaatggattactgg |
| | | ggacagggtactctggtcaccgtgtccagcaccactaccccagcaccgaggccacccaccc |
| | | cggctcctaccatcgcctcccagcctctgtccctgcgtccggaggcatgtagacccgcagct |
| | | ggtggggccgtgcatacccggggtcttgacttcgcctgcgatatctacatttgggcccctctgg |
| | | ctggtacttgcggggtcctgctgctttcactcgtgatcactctttactgtaagcgcggtcggaag |
| | | aagctgctgtacatctttaagcaacccttcatgaggcctgtgcagactactcaagaggaggac |
| | | ggctgttcatgccggttcccagaggaggaggaaggcggctgcgaactgcgcgtgaaattca |
| | | gccgcagcgcagatgctccagcctaccagcaggggcagaaccagctctacaacgaactca |
| | | atcttggtcggagagaggagtacgacgtgctggacaagcggagaggacgggacccagaaa |
| | | tgggcgggaagccgcgcagaaagaatccccaagagggcctgtacaacgagctccaaaag |
| | | gataagatggcagaagcctatagcgagattggtatgaaaggggaacgcagaagaggcaaa |
| | | ggccacgacggactgtaccagggactcagcaccgccaccaaggacacctatgacgctcttc |
| | | acatgcaggccctgccgcctcgg |
| | 818 | MALPVTALLLPLALLLHAARPQSALTQPASASGSPGQSVTI |
| | | SCTGTSSDVGGYNYVSWYQQHPGKAPKLMIYDVSNRPSG |
| | | VSNRFSGSKSGNTASLTISGLQAEDEADYYCSSYTSSSTLY |
| | | VFGTGTQLTVLGGGGSEVQLQQSGPGLVKPSQTLSLTCAIS |
| | | GDSMLSNSDTWNWIRQSPSRGLEWLGRTYHRSTWYDDYA |
| | | SSVRGRVSINVDTSKNQYSLQLNAVTPEDTGVYYCARVRL |
| | | QDGNSWSDAFDVWGQGTMVTVSSGGGGSEIVMTQSPATL |
| | | SLSPGERATLSCRASQDISKYLNWYQQKPGQAPRLLIYHTS |
| | | RLHSGIPARFSGSGSGTDYTLTISSLQPEDFAVYFCQQGNTL |
| | | PYTFGQGTKLEIKGGGGSGGGGSGGGGSQVQLQESGPGLV |
| | | KPSETLSLTCTVSGVSLPDYGVSWIRQPPGKGLEWIGVIWG |
| | | SETTYYQSSLKSRVTISKDNSKNQVSLKLSSVTAADTAVYY |
| | | CAKHYYYGGSYAMDYWGQGTLVTVSSTTTPAPRPPTPAPT |
| | | IASQPLSLRPEACRPAAGGAVHTRGLDFACDIYIWAPLAGT |
| | | CGVLLLSLVITLYCKRGRKKLLYIFKQPFMRPVQTTQEEDG |
| | | CSCRFPEEEEGGCELRVKFSRSADAPAYQQGQNQLYNELN |
| | | LGRREEYDVLDKRRGRDPEMGGKPRRKNPQEGLYNELQK |
| | | DKMAEAYSEIGMKGERRRGKGHDGLYQGLSTATKDTYDA |
| | | LHMQALPPR |
| CG#c224 | 819 | atggccctgcccgtgactgcgctcctgcttccgttggccctgctcctgcatgccgccagacctc |
| | | agtccgctctgactcagccggcctcagcttcggggtcccctggtcaaagcgtcactatttcctg |
| | | taccggaacctcatcagacgtgggcggctacaattacgtgtcctggtaccaacagcaccccg |
| | | gaaaggctcctaagcttatgatctacgacgtgtccaaccggccgtcaggagtgtccaacagat |
| | | tctccggctccaagagcggaaacactgccagcttgaccattagcggcttgcaggccgagga |
| | | cgaagccgactactactgctctagctacacatcctcgtctaccctctacgtgtttggaacgggg |
| | | acccagctgactgtgctcgggggtggaggatcagaggtgcaactccagcagtccggtcctg |
| | | gcctcgtgaaaccgtcccaaaccctgtccctgacttgcgccatctcgggcgactccatgctgt |
| | | ccaattccgacacctggaactggattagacaatcgcctagccggggactcgaatggctgggc |
| | | cggacctaccaccggtccacgtggtatgacgactacgcaagctccgtccggggaagggtgt |
| | | ccattaacgtcgatacctccaagaaccagtacagccttcagctgaacgctgtgacccccgag |
| | | gataccggcgtctactactgtgcaagagtgcgattgcaggatggaaactcgtggtcggacgc |
| | | attcgatgtctggggacagggaactatggtgaccgtgtcctcgggcggaggcgggagcgga |
| | | ggaggaggctctggcggaggaggaagcgagattgtcatgactcagtccccggccacactct |
| | | ccctgtcacccggagaaagagcaaccctgagctgcagggcgtcccaggacatctcgaagta |
| | | cctgaactggtaccagcagaagcctggacaagcaccccgcctcctgatctaccacacctcgc |
| | | ggctgcattcgggaatccccgccagattctcagggagcggatcaggaaccgactacaccct |
| | | gactatctcgagcctgcaaccagaggatttcgccgtgtacttctgccagcaaggaaacaccct |
| | | gccctacacctttggacagggaaccaagctcgagattaaggggggtggtggatcgggaggg |
| | | ggtggatcaggaggaggcggctcacaagtccagctgcaagaatccggtccgggacttgtga |
| | | agccgtccgaaaccctgtcactgacttgcactgtgtccggggtgtcattgcccgactacggcg |
| | | tgagctggattcggcagccccctggaaagggattggaatggatcggcgtgatctggggttcg |
| | | gaaactacctactatcagtcctcactgaagtcccgcgtgaccatcagcaaggataattccaaaa |
| | | accaagtgtctctgaagctctccagcgtcactgccgccgatactgccgtgtactactgcgcca |
| | | agcactactattacggcggttcgtacgccatggactactggggccaagggacactcgtgacc |
| | | gtgtcatccaccactaccccagcaccgaggccacccaccccggctcctaccatcgcctccca |
| | | gcctctgtccctgcgtccggaggcatgtagacccgcagctggtggggccgtgcatacccgg |
| | | ggtcttgacttcgcctgcgatatctacatttgggcccctctggctggtacttgcggggtcctgct |
| | | gctttcactcgtgatcactctttactgtaagcgcggtcggaagaagctgctgtacatctttaagca |
| | | acccttcatgaggcctgtgcagactactcaagaggaggacggctgttcatgccggttcccaga |
| | | ggaggaggaaggcggctgcgaactgcgcgtgaaattcagccgcagcgcagatgctccagc |
| | | ctaccagcaggggcagaaccagctctacaacgaactcaatcttggtcggagagaggagtac |
| | | gacgtgctggacaagcggagaggacgggacccagaaatgggcgggaagccgcgcagaa |
| | | agaatccccaagagggcctgtacaacgagctccaaaaggataagatggcagaagcctatag |
| | | cgagattggtatgaaaggggaacgcagaagaggcaaaggccacgacggactgtaccagg |
| | | gactcagcaccgccaccaaggacacctatgacgctcttcacatgcaggccctgccgcctcgg |
| | 820 | MALPVTALLLPLALLLHAARPQSALTQPASASGSPGQSVTI |
| | | SCTGTSSDVGGYNYVSWYQQHPGKAPKLMIYDVSNRPSG |
| | | VSNRFSGSKSGNTASLTISGLQAEDEADYYCSSYTSSSTLY |
| | | VFGTGTQLTVLGGGGSEVQLQQSGPGLVKPSQTLSLTCAIS |
| | | GDSMLSNSDTWNWIRQSPSRGLEWLGRTYHRSTWYDDYA |
| | | SSVRGRVSINVDTSKNQYSLQLNAVTPEDTGVYYCARVRL |
| | | QDGNSWSDAFDVWGQGTMVTVSSGGGGSGGGGSGGGGS |
| | | EIVMTQSPATLSLSPGERATLSCRASQDISKYLNWYQQKPG |
| | | QAPRLLIYHTSRLHSGIPARFSGSGSGTDYTLTISSLQPEDFA |
| | | VYFCQQGNTLPYTFGQGTKLEIKGGGGSGGGGSGGGGSQV |
| | | QLQESGPGLVKPSETLSLTCTVSGVSLPDYGVSWIRQPPGK |
| | | GLEWIGVIWGSETTYYQSSLKSRVTISKDNSKNQVSLKLSS |
| | | VTAADTAVYYCAKHYYYGGSYAMDYWGQGTLVTVSSTT |
| | | TPAPRPPTPAPTIASQPLSLRPEACRPAAGGAVHTRGLDFAC |
| | | DIYIWAPLAGTCGVLLLSLVITLYCKRGRKKLLYIFKQPFM |
| | | RPVQTTQEEDGCSCRFPEEEEGGCELRVKFSRSADAPAYQQ |
| | | GQNQLYNELNLGRREEYDVLDKRRGRDPEMGGKPRRKNP |
| | | QEGLYNELQKDKMAEAYSEIGMKGERRRGKGHDGLYQGL |
| | | STATKDTYDALHMQALPPR |
| CG#c227 | 821 | atggccctgcccgtgactgcgctcctgcttccgttggccctgctcctgcatgccgccagacctc |
| | | agtccgctctgactcagccggcctcagcttcggggtcccctggtcaaagcgtcactatttcctg |
| | | taccggaacctcatcagacgtgggcggctacaattacgtgtcctggtaccaacagcaccccg |
| | | gaaaggctcctaagcttatgatctacgacgtgtccaaccggccgtcaggagtgtccaacagat |
| | | tctccggctccaagagcggaaacactgccagcttgaccattagcggcttgcaggccgagga |
| | | cgaagccgactactactgctctagctacacatcctcgtctaccctctacgtgtttggaacgggg |
| | | acccagctgactgtgctcgggggtggaggatcagaggtgcaactccagcagtccggtcctg |
| | | gcctcgtgaaaccgtcccaaaccctgtccctgacttgcgccatctcgggcgactccatgctgt |
| | | ccaattccgacacctggaactggattagacaatcgcctagccggggactcgaatggctgggc |
| | | cggacctaccaccggtccacgtggtatgacgactacgcaagctccgtccggggaagggtgt |
| | | ccattaacgtcgatacctccaagaaccagtacagccttcagctgaacgctgtgacccccgag |
| | | gataccggcgtctactactgtgcaagagtgcgattgcaggatggaaactcgtggtcggacgc |
| | | attcgatgtctggggacagggaactatggtcactgtgtcctccggcggtggaggctcggggg |
| | | ggggcggctcaggaggaggcggctcacaagtccagctgcaagaatccggtccgggacttg |
| | | tgaagccgtccgaaaccctgtcactgacttgcactgtgtccggggtgtcattgcccgactacg |
| | | gcgtgagctggattcggcagccccctggaaagggattggaatggatcggcgtgatctggggt |
| | | tcggaaactacctactatcagtcctcactgaagtcccgcgtgaccatcagcaaggataattcca |
| | | aaaaccaagtgtctctgaagctctccagcgtcactgccgccgatactgccgtgtactactgcg |
| | | ccaagcactactattacggcggttcgtacgccatggactactggggacaaggcactcttgtga |
| | | ctgtgtcaagcggcggtggagggagcggtgggggcggttcaggaggaggcggatcagag |
| | | atcgtgatgacccaatccccagccaccctgtccctcagccctggagaaagagccaccctgag |
| | | ctgccgggcctcccaggatatcagcaagtacttgaactggtaccaacaaaagccggggcag |
| | | gcgccccggctcctgatctaccacacctcgcgcctccactcaggtatccccgccagattctca |
| | | gggagcggctccggtactgactacaccctgactatttcctcactgcagccagaggactttgcc |
| | | gtgtacttctgccagcagggaaacactctgccgtacaccttcgggcagggaacgaagcttga |
| | | aattaagaccactaccccagcaccgaggccacccaccccggctcctaccatcgcctcccag |
| | | cctctgtccctgcgtccggaggcatgtagacccgcagctggtggggccgtgcatacccggg |
| | | gtcttgacttcgcctgcgatatctacatttgggcccctctggctggtacttgcggggtcctgctg |
| | | ctttcactcgtgatcactctttactgtaagcgcggtcggaagaagctgctgtacatctttaagcaa |
| | | cccttcatgaggcctgtgcagactactcaagaggaggacggctgttcatgccggttcccaga |
| | | ggaggaggaaggcggctgcgaactgcgcgtgaaattcagccgcagcgcagatgctccagc |
| | | ctaccagcaggggcagaaccagctctacaacgaactcaatcttggtcggagagaggagtac |
| | | gacgtgctggacaagcggagaggacgggacccagaaatgggcgggaagccgcgcagaa |
| | | agaatccccaagagggcctgtacaacgagctccaaaaggataagatggcagaagcctatag |
| | | cgagattggtatgaaaggggaacgcagaagaggcaaaggccacgacggactgtaccagg |
| | | gactcagcaccgccaccaaggacacctatgacgctcttcacatgcaggccctgccgcctcgg |
| | 822 | MALPVTALLLPLALLLHAARPQSALTQPASASGSPGQSVTI |
| | | SCTGTSSDVGGYNYVSWYQQHPGKAPKLMIYDVSNRPSG |
| | | VSNRFSGSKSGNTASLTISGLQAEDEADYYCSSYTSSSTLY |
| | | VFGTGTQLTVLGGGGSEVQLQQSGPGLVKPSQTLSLTCAIS |
| | | GDSMLSNSDTWNWIRQSPSRGLEWLGRTYHRSTWYDDYA |
| | | SSVRGRVSINVDTSKNQYSLQLNAVTPEDTGVYYCARVRL |
| | | QDGNSWSDAFDVWGQGTMVTVSSGGGGSGGGGSGGGGS |
| | | QVQLQESGPGLVKPSETLSLTCTVSGVSLPDYGVSWIRQPP |
| | | GKGLEWIGVIWGSETTYYQSSLKSRVTISKDNSKNQVSLKL |
| | | SSVTAADTAVYYCAKHYYYGGSYAMDYWGQGTLVTVSS |
| | | GGGGSGGGGSGGGGSEIVMTQSPATLSLSPGERATLSCRAS |
| | | QDISKYLNWYQQKPGQAPRLLIYHTSRLHSGIPARFSGSGS |
| | | GTDYTLTISSLQPEDFAVYFCQQGNTLPYTFGQGTKLEIKTT |
| | | TPAPRPPTPAPTIASQPLSLRPEACRPAAGGAVHTRGLDFAC |
| | | DIYIWAPLAGTCGVLLLSLVITLYCKRGRKKLLYIFKQPFM |
| | | RPVQTTQEEDGCSCRFPEEEEGGCELRVKFSRSADAPAYQQ |
| | | GQNQLYNELNLGRREEYDVLDKRRGRDPEMGGKPRRKNP |
| | | QEGLYNELQKDKMAEAYSEIGMKGERRRGKGHDGLYQGL |
| | | STATKDTYDALHMQALPPR |

Dual CD19-CD22 CARS
CG#c201

| | | |
|---|---|---|
| Full length | 823 | atggccctccctgtcaccgccctgctgcttccgctggctcttctgctccacgccgctcggcccg |
| CD19-CD22 | | aagtgcagctgcagcagtcagggcctggcctggtcaagccgtcgcagaccctctccctgac |
| Dual CAR | | atgcgccattagcggggactccatgctgagcaactcggacacctggaactggattcggcagt |
| nucleic acid | | ccccttcccggggactcgagtggctcggacgcacctaccatcggagcacttggtacgacga |
| | | ctacgcctcctccgtgagaggtcgcgtgtcgatcaacgtggatacctcgaagaaccagtatag |
| | | cttgcaactgaacgccgtgacccctgaggataccggagtgtactattgtgcgagagtcaggct |
| | | gcaagacggaaactcctggtccgacgcatttgatgtctggggacagggtactatggtcacggt |
| | | gtcatctggaggcggaggatcgcaaagcgccctgactcagccggcttcggctagcggttca |
| | | ccggggcagtccgtgactatctcctgcaccgggacttcctccgacgtgggaggctacaattac |
| | | gtgtcctggtaccagcaacaccccggcaaagccccaaagctgatgatctacgacgtcagcaa |
| | | cagacccagcggagtgtccaaccggttcagcggctccaagtccggcaacaccgcctccctg |
| | | accatcagcgggcttcaggccgaagatgaggcggattactactgctcctcgtacacctcaag |
| | | ctcaactctgtacgtgttcggcaccggtactcagctcaccgtgctgaccactaccccagcacc |
| | | gaggccacccaccccggctcctaccatcgcctcccagcctctgtccctgcgtccggaggcat |
| | | gtagacccgcagctggtggggccgtgcatacccggggtcttgacttcgcctgcgatatctaca |
| | | tttgggcccctctggctggtacttgcggggtcctgctgctttcactcgtgatcactctttactgtaa |
| | | gcgcggtcggaagaagctgctgtacatctttaagcaacccttcatgaggcctgtgcagactac |
| | | tcaagaggaggacggctgttcatgccggttcccagaggaggaggaaggcggctgcgaact |
| | | gcgcgtgaaattcagccgcagcgcagatgctccagcctaccagcaggggcagaaccagct |
| | | ctacaacgaactcaatcttggtcggagagaggagtacgacgtgctggacaagcggagagga |
| | | cgggacccagaaatgggcgggaagccgcgcagaaagaatccccaagagggcctgtacaa |
| | | cgagctccaaaaggataagatggcagaagcctatagcgagattggtatgaaaggggaacgc |
| | | agaagaggcaaaggccacgacggactgtaccagggactcagcaccgccaccaaggacac |
| | | ctatgacgctcttcacatgcaggccctgccgcctcggggaagcggagctactaacttcagcct |
| | | gctgaagcaggctggagacgtggaggagaaccctggacctatggccttaccagtgaccgcc |
| | | ttgctcctgccgctggccttgctgctccacgccgccaggccggaaattgtgatgacccagtca |
| | | cccgccactcttagcctttcacccggtgagcgcgcaaccctgtcttgcagagcctcccaagac |
| | | atctcaaaataccttaattggtatcaacagaagcccggacaggctcctcgccttctgatctacca |
| | | caccagccggctccattctggaatccctgccaggttcagcggtagcggatctgggaccgact |
| | | acaccctcactatcagctcactgcagccagaggacttcgctgtctatttctgtcagcaagggaa |
| | | caccctgccctacacctttggacagggcaccaagctcgagattaaaggtggaggtggcagc |
| | | ggaggaggtgggtccggcggtggaggaagccaggtccaactccaagaaagcggaccgg |
| | | gtcttgtgaagccatcagaaactctttcactgacttgtactgtgagcggagtgtctctccccgatt |
| | | acggggtgtcttggatcagacagccaccggggaagggtctggaatggattggagtgatttgg |
| | | ggctctgagactacttactaccaatcatccctcaagtcacgcgtcaccatctcaaaggacaact |
| | | ctaagaatcaggtgtcactgaaactgtcatctgtgaccgcagccgacaccgccgtgtactattg |
| | | cgctaagcattactattatggcgggagctacgcaatggattactggggacagggtactctggt |
| | | caccgtgtccagcaccacgacgccagcgccgcgaccaccaacaccggcgcccaccatcg |
| | | cgtcgcagcccctgtccctgcgcccagaggcgtgccggccagcggcggggggcgcagtg |
| | | cacacgagggggctggacttcgcctgtgatatctacatctgggcgcccttggccgggacttgt |
| | | ggggtccttctcctgtcactggttatcaccctttactgcaaacggggcagaaagaaactcctgt |
| | | atatattcaaacaaccatttatgagaccagtacaaactactcaagaggaagatggctgtagctg |
| | | ccgatttccagaagaagaagaaggaggatgtgaactgagagtgaagttcagcaggagcgca |
| | | gacgcccccgcgtaccagcagggccagaaccagctctataacgagctcaatctaggacgaa |
| | | gagaggagtacgatgttttggacaagagacgtggccgggaccctgagatggggggaaagc |
| | | cgagaaggaagaaccctcaggaaggcctgtacaatgaactgcagaaagataagatggcgg |
| | | aggcctacagtgagattgggatgaaaggcgagcgccggaggggcaaggggcacgatggc |
| | | ctttaccagggtctcagtacagccaccaaggacacctacgacgcccttcacatgcaggccct |
| | | gccccctcgc |
| Full length | 824 | MALPVTALLLPLALLLHAARPEVQLQQSGPGLVKPSQTLSL |
| CD19-CD22 | | TCAISGDSMLSNSDTWNWIRQSPSRGLEWLGRTYHRSTWY |
| Dual CAR | | DDYASSVRGRVSINVDTSKNQYSLQLNAVTPEDTGVYYCA |
| amino acid | | RVRLQDGNSWSDAFDVWGQGTMVTVSSGGGGSQSALTQP |
| | | ASASGSPGQSVTISCTGTSSDVGGYNYVSWYQQHPGKAPK |
| | | LMIYDVSNRPSGVSNRFSGSKSGNTASLTISGLQAEDEADY |
| | | YCSSYTSSSTLYVFGTGTQLTVLTTTPAPRPPTPAPTIASQPL |
| | | SLRPEACRPAAGGAVHTRGLDFACDIYIWAPLAGTCGVLL |
| | | LSLVITLYCKRGRKKLLYIFKQPFMRPVQTTQEEDGCSCRF |
| | | PEEEEGGCELRVKFSRSADAPAYQQGQNQLYNELNLGRRE |
| | | EYDVLDKRRGRDPEMGGKPRRKNPQEGLYNELQKDKMA |
| | | EAYSEIGMKGERRRGKGHDGLYQGLSTATKDTYDALHMQ |
| | | ALPPRGSGATNFSLLKQAGDVEENPGPMALPVTALLLPLAL |
| | | LLHAARPEIVMTQSPATLSLSPGERATLSCRASQDISKYLN |
| | | WYQQKPGQAPRLLIYHTSRLHSGIPARFSGSGSGTDYTLTIS |
| | | SLOPEDFAVYFCQQGNTLPYTFGQGTKLEIKGGGGSGGGG |
| | | SGGGGSQVQLQESGPGLVKPSETLSLTCTVSGVSLPDYGVS |
| | | WIRQPPGKGLEWIGVIWGSETTYYQSSLKSRVTISKDNSKN |
| | | QVSLKLSSVTAADTAVYYCAKHYYYGGSYAMDYWGQGT |
| | | LVTVSSTTTPAPRPPTPAPTIASQPLSLRPEACRPAAGGAVH |
| | | TRGLDFACDIYIWAPLAGTCGVLLLSLVITLYCKRGRKKLL |
| | | YIFKQPFMRPVQTTQEEDGCSCRFPEEEEGGCELRVKFSRS |
| | | ADAPAYQQGQNQLYNELNLGRREEYDVLDKRRGRDPEM |
| | | GGKPRRKNPQEGLYNELQKDKMAEAYSEIGMKGERRRGK |
| | | GHDGLYQGLSTATKDTYDALHMQALPPR |
| CD22 CAR | 825 | MALPVTALLLPLALLLHAARPEVQLQQSGPGLVKPSQTLSL |
| (with P2A | | TCAISGDSMLSNSDTWNWIRQSPSRGLEWLGRTYHRSTWY |
| site) | | DDYASSVRGRVSINVDTSKNQYSLQLNAVTPEDTGVYYCA |
| | | RVRLQDGNSWSDAFDVWGQGTMVTVSSGGGGSQSALTQP |
| | | ASASGSPGQSVTISCTGTSSDVGGYNYVSWYQQHPGKAPK |
| | | LMIYDVSNRPSGVSNRFSGSKSGNTASLTISGLQAEDEADY |
| | | YCSSYTSSSTLYVFGTGTQLTVLTTTPAPRPPTPAPTIASQPL |
| | | SLRPEACRPAAGGAVHTRGLDFACDIYIWAPLAGTCGVLL |
| | | LSLVITLYCKRGRKKLLYIFKQPFMRPVQTTQEEDGCSCRF |
| | | PEEEEGGCELRVKFSRSADAPAYQQGQNQLYNELNLGRRE |
| | | EYDVLDKRRGRDPEMGGKPRRKNPQEGLYNELQKDKMA |
| | | EAYSEIGMKGERRRGKGHDGLYQGLSTATKDTYDALHMQ |
| | | ALPPRGSGATNFSLLKQAGDVEENPG |
| CD19 CAR | 826 | PMALPVTALLLPLALLLHAARPEIVMTQSPATLSLSPGERA |
| | | TLSCRASQDISKYLNWYQQKPGQAPRLLIYHTSRLHSGIPA |
| | | RFSGSGSGTDYTLTISSLQPEDFAVYFCQQGNTLPYTFGQG |
| | | TKLEIKGGGGSGGGGSGGGGSQVQLQESGPGLVKPSETLSL |
| | | TCTVSGVSLPDYGVSWIRQPPGKGLEWIGVIWGSETTYYQS |
| | | SLKSRVTISKDNSKNQVSLKLSSVTAADTAVYYCAKHYYY |
| | | GGSYAMDYWGQGTLVTVSSTTTPAPRPPTPAPTIASQPLSL |
| | | RPEACRPAAGGAVHTRGLDFACDIYIWAPLAGTCGVLLLS |
| | | LVITLYCKRGRKKLLYIFKQPFMRPVQTTQEEDGCSCRFPE |
| | | EEEGGCELRVKFSRSADAPAYQQGQNQLYNELNLGRREEY |
| | | DVLDKRRGRDPEMGGKPRRKNPQEGLYNELQKDKMAEA |
| | | YSEIGMKGERRRGKGHDGLYQGLSTATKDTYDALHMQAL |
| | | PPR |

CG#c203

| | | |
|---|---|---|
| Full length | 827 | atggccttaccagtgaccgccttgctcctgccgctggccttgctgctccacgccgccaggccg |
| CD19-CD22 | | gaaattgtgatgacccagtcacccgccactcttagcctttcacccggtgagcgcgcaaccctg |
| Dual CAR | | tcttgcagagcctcccaagacatctcaaaataccttaattggtatcaacagaagcccggacag |
| nucleic acid | | gctcctcgccttctgatctaccacaccagccggctccattctggaatccctgccaggttcagcg |
| | | gtagcggatctgggaccgactacaccctcactatcagctcactgcagccagaggacttcgct |
| | | gtctatttctgtcagcaagggaacaccctgccctacacctttggacagggcaccaagctcgag |
| | | attaaaggtggaggtggcagcggaggaggtgggtccggcggtggaggaagccaggtcca |
| | | actccaagaaagcggaccgggtcttgtgaagccatcagaaactctttcactgacttgtactgtg |
| | | agcggagtgtctctccccgattacggggtgtcttggatcagacagccaccggggaagggtct |
| | | ggaatggattggagtgatttggggctctgagactacttactaccaatcatccctcaagtcacgc |
| | | gtcaccatctcaaaggacaactctaagaatcaggtgtcactgaaactgtcatctgtgaccgcag |
| | | ccgacaccgccgtgtactattgcgctaagcattactattatggcgggagctacgcaatggatta |
| | | ctggggacagggtactctggtcaccgtgtccagcaccacgacgccagcgccgcgaccacc |
| | | aacaccggcgcccaccatcgcgtcgcagcccctgtccctgcgcccagaggcgtgccggcc |
| | | agcggcggggggcgcagtgcacacgagggggctggacttcgcctgtgatatctacatctgg |
| | | gcgcccttggccgggacttgtggggtccttctcctgtcactggttatcaccctttactgcaaacg |
| | | gggcagaaagaaactcctgtatatattcaaacaaccatttatgagaccagtacaaactactcaa |
| | | gaggaagatggctgtagctgccgatttccagaagaagaagaaggaggatgtgaactgagag |
| | | tgaagttcagcaggagcgcagacgcccccgcgtaccagcagggccagaaccagctctata |
| | | acgagctcaatctaggacgaagagaggagtacgatgttttggacaagagacgtggccggga |
| | | ccctgagatggggggaaagccgagaaggaagaaccctcaggaaggcctgtacaatgaact |
| | | gcagaaagataagatggcggaggcctacagtgagattgggatgaaaggcgagcgccgga |
| | | ggggcaaggggcacgatggcctttaccagggtctcagtacagccaccaaggacacctacga |
| | | cgcccttcacatgcaggccctgccccctcgcggaagcggagctactaacttcagcctgctga |
| | | agcaggctggagacgtggaggagaaccctggacctatggccctccctgtcaccgccctgct |
| | | gcttccgctggctcttctgctccacgccgctcggcccgaagtgcagctgcagcagtcagggc |
| | | ctggcctggtcaagccgtcgcagaccctctccctgacatgcgccattagcggggactccatg |
| | | ctgagcaactcggacacctggaactggattcggcagtccccttcccggggactcgagtggct |
| | | cggacgcacctaccatcggagcacttggtacgacgactacgcctcctccgtgagaggtcgc |
| | | gtgtcgatcaacgtggatacctcgaagaaccagtatagcttgcaactgaacgccgtgacccct |
| | | gaggataccggagtgtactattgtgcgagagtcaggctgcaagacggaaactcctggtccga |
| | | cgcatttgatgtctggggacagggtactatggtcacggtgtcatctggaggcggaggatcgca |
| | | aagcgccctgactcagccggcttcggctagcggttcaccggggcagtccgtgactatctcct |
| | | gcaccgggacttcctccgacgtgggaggctacaattacgtgtcctggtaccagcaacacccc |
| | | ggcaaagccccaaagctgatgatctacgacgtcagcaacagacccagcggagtgtccaacc |
| | | ggttcagcggctccaagtccggcaacaccgcctccctgaccatcagcgggcttcaggccga |
| | | agatgaggcggattactactgctcctcgtacacctcaagctcaactctgtacgtgttcggcacc |
| | | ggtactcagctcaccgtgctgaccactaccccagcaccgaggccacccaccccggctccta |
| | | ccatcgcctcccagcctctgtccctgcgtccggaggcatgtagacccgcagctggtggggcc |
| | | gtgcatacccggggtcttgacttcgcctgcgatatctacatttgggcccctctggctggtacttg |
| | | cggggtcctgctgctttcactcgtgatcactctttactgtaagcgcggtcggaagaagctgctgt |
| | | acatctttaagcaacccttcatgaggcctgtgcagactactcaagaggaggacggctgttcatg |
| | | ccggttcccagaggaggaggaaggggctgcgaactgcgcgtgaaattcagccgcagcgc |
| | | agatgctccagcctaccagcaggggcagaaccagctctacaacgaactcaatcttggtcgga |
| | | gagaggagtacgacgtgctggacaagcggagaggacgggacccagaaatgggcgggaa |
| | | gccgcgcagaaagaatccccaagagggcctgtacaacgagctccaaaaggataagatggc |
| | | agaagcctatagcgagattggtatgaaaggggaacgcagaagaggcaaaggccacgacg |
| | | gactgtaccagggactcagcaccgccaccaaggacacctatgacgctcttcacatgcaggcc |
| | | ctgccgcctcgg |
| Full length | 828 | MALPVTALLLPLALLLHAARPEIVMTQSPATLSLSPGERAT |
| CD19-CD22 | | LSCRASQDISKYLNWYQQKPGQAPRLLIYHTSRLHSGIPAR |
| Dual CAR | | FSGSGSGTDYTLTISSLQPEDFAVYFCQQGNTLPYTFGQGT |
| amino acid | | KLEIKGGGGSGGGGSGGGGSQVQLQESGPGLVKPSETLSLT |
| | | CTVSGVSLPDYGVSWIRQPPGKGLEWIGVIWGSETTYYQSS |
| | | LKSRVTISKDNSKNQVSLKLSSVTAADTAVYYCAKHYYYG |
| | | GSYAMDYWGQGTLVTVSSTTTPAPRPPTPAPTIASQPLSLR |
| | | PEACRPAAGGAVHTRGLDFACDIYIWAPLAGTCGVLLLSL |
| | | VITLYCKRGRKKLLYIFKQPFMRPVQTTQEEDGCSCRFPEE |
| | | EEGGCELRVKFSRSADAPAYQQGQNQLYNELNLGRREEY |
| | | DVLDKRRGRDPEMGGKPRRKNPQEGLYNELQKDKMAEA |
| | | YSEIGMKGERRRGKGHDGLYQGLSTATKDTYDALHMQAL |
| | | PPRGSGATNFSLLKQAGDVEENPGPMALPVTALLLPLALLL |
| | | HAARPEVQLQQSGPGLVKPSQTLSLTCAISGDSMLSNSDT |
| | | WNWIRQSPSRGLEWLGRTYHRSTWYDDYASSVRGRVSIN |
| | | VDTSKNQYSLQLNAVTPEDTGVYYCARVRLQDGNSWSDA |
| | | FDVWGQGTMVTVSSGGGGSQSALTQPASASGSPGQSVTIS |
| | | CTGTSSDVGGYNYVSWYQQHPGKAPKLMIYDVSNRPSGV |
| | | SNRFSGSKSGNTASLTISGLQAEDEADYYCSSYTSSSTLYVF |
| | | GTGTQLTVLTTTPAPRPPTPAPTIASQPLSLRPEACRPAAGG |
| | | AVHTRGLDFACDIYIWAPLAGTCGVLLLSLVITLYCKRGRK |
| | | KLLYIFKQPFMRPVQTTQEEDGCSCRFPEEEEGGCELRVKF |
| | | SRSADAPAYQQGQNQLYNELNLGRREEYDVLDKRRGRDP |
| | | EMGGKPRRKNPQEGLYNELQKDKMAEAYSEIGMKGERRR |
| | | GKGHDGLYQGLSTATKDTYDALHMQALPPR |
| CD19 CAR | 829 | MALPVTALLLPLALLLHAARPEIVMTQSPATLSLSPGERAT |
| (with P2A | | LSCRASQDISKYLNWYQQKPGQAPRLLIYHTSRLHSGIPAR |
| site) | | FSGSGSGTDYTLTISSLQPEDFAVYFCQQGNTLPYTFGQGT |
| | | KLEIKGGGGSGGGGSGGGGSQVQLQESGPGLVKPSETLSLT |
| | | CTVSGVSLPDYGVSWIRQPPGKGLEWIGVIWGSETTYYQSS |
| | | LKSRVTISKDNSKNQVSLKLSSVTAADTAVYYCAKHYYYG |
| | | GSYAMDYWGQGTLVTVSSTTTPAPRPPTPAPTIASQPLSLR |
| | | PEACRPAAGGAVHTRGLDFACDIYIWAPLAGTCGVLLLSL |
| | | VITLYCKRGRKKLLYIFKQPFMRPVQTTQEEDGCSCRFPEE |
| | | EEGGCELRVKFSRSADAPAYQQGQNQLYNELNLGRREEY |
| | | DVLDKRRGRDPEMGGKPRRKNPQEGLYNELQKDKMAEA |
| | | YSEIGMKGERRRGKGHDGLYQGLSTATKDTYDALHMQAL |
| | | PPRGSGATNFSLLKQAGDVEENPG |
| CD22 CAR | 830 | PMALPVTALLLPLALLLHAARPEVQLQQSGPGLVKPSQTLS |
| | | LTCAISGDSMLSNSDTWNWIRQSPSRGLEWLGRTYHRSTW |
| | | YDDYASSVRGRVSINVDTSKNQYSLQLNAVTPEDTGVYYC |
| | | ARVRLQDGNSWSDAFDVWGQGTMVTVSSGGGGSQSALT |
| | | QPASASGSPGQSVTISCTGTSSDVGGYNYVSWYQQHPGKA |
| | | PKLMIYDVSNRPSGVSNRFSGSKSGNTASLTISGLQAEDEA |
| | | DYYCSSYTSSSTLYVFGTGTQLTVLTTTPAPRPPTPAPTIAS |
| | | QPLSLRPEACRPAAGGAVHTRGLDFACDIYIWAPLAGTCG |
| | | VLLLSLVITLYCKRGRKKLLYIFKQPFMRPVQTTQEEDGCS |
| | | CRFPEEEEGGCELRVKFSRSADAPAYQQGQNQLYNELNLG |
| | | RREEYDVLDKRRGRDPEMGGKPRRKNPQEGLYNELQKDK |
| | | MAEAYSEIGMKGERRRGKGHDGLYQGLSTATKDTYDALH |
| | | MQALPPR |

CG#c230

| | | |
|---|---|---|
| Full length | 831 | atggcacttcccgtcaccgccctgctgctcccactcgccctccttctgcacgccgcccgcccc |
| CD19-CD22 | | gaagtgcagctgcagcagtcaggaccgggcctggtcaaaccttcgcagactctgtccctgac |
| Dual CAR | | ttgcgctataagcggggactccatgctgagcaattcggacacttggaactggattcgccaaag |
| nucleic acid | | ccccagccggggtctggaatggctgggaaggacctaccatcgctctacttggtacgacgact |
| | | acgccagctccgtgcgaggacgcgtgtccatcaacgtggacacctccaagaaccagtactc |
| | | gcttcaactcaacgcagtgacccctgaagataccggagtctactattgcgcccgcgtgcggct |
| | | ccaggacgggaactcctggtcggacgctttcgatgtctggggacagggcactatggtcaccg |
| | | tcagctccggcggcggcggtagccaatcggcgctgacacagccggcttccgcctcgggatc |
| | | gcctggacagtcggtgaccatctcgtgcactggaacctcctccgacgtgggcggctacaatta |
| | | tgtgtcatggtaccagcagcacccgggaaaggcccctaagctgatgatctacgacgtgtcca |
| | | atagacctagcggggtgtcaaacagattctccggatccaaatccggaaacactgcctccctga |
| | | ccatttccggactgcaggccgaggacgaagccgattactactgctcctcttacacctcctcatc |
| | | caccctctacgtgtttgggactgggacccagctgaccgtcctcactaccaccccggccccgc |
| | | ggccccctacaccggcaccgactattgccagccagcctctctcgctgcggccggaggcctg |
| | | ccgcccagccgccggcggagccgtgcacacccgcggtctggacttcgcgtgcgatatctac |
| | | atctgggctccgctggccgggacttgtggcgtgctgctgctgtctctggtcatcacactgtact |
| | | gcaagcgcggaagaaagaagctgctctacatcttcaagcaacccttcatgcggcctgtgcag |
| | | accacccaggaagaggatggctgctcctgccggttcccggaggaagaagagggcggatgc |
| | | gaactgcgcgtgaagttcagccgaagcgccgacgccccggcctaccagcagggccagaa |
| | | ccaactgtacaacgaactcaacctgggtcggagagaagagtacgacgtgctggacaaaaga |
| | | cgcggcagggaccccgagatgggcggaaagcctcgccgcaagaacccgcaggagggcc |
| | | tctacaacgagctgcagaaggacaagatggccgaagcctactcagagatcggcatgaaggg |
| | | ggagcggaggcgcgggaagggccacgacggtttgtaccaaggactttccactgcgaccaa |
| | | ggacacctacgatgccctccatatgcaagccctgccgccccggggttccggagctaccaact |
| | | tctcgctgttgaagcaggccggagatgtcgaggaaaacccgggacctatggccctgccagt |
| | | gaccgcgctcctgctgcccctggctctgctgcttcacgcggcccggcctgagattgtgatgac |
| | | tcagagcccggcgaccctgtccctgtcccccggggagagagcaaccctgtcgtgccgggc |
| | | ctcccaagacatctcaaagtacctcaattggtatcagcagaagccaggacaggctccacggtt |
| | | gctgatctaccacacttcgagactgcactcaggaatccccgcgcggttttccggttccggctcc |
| | | gggaccgactacaccctgaccatcagctcgctccagcctgaggatttcgcagtgtacttctgtc |
| | | agcaaggaaacacccttccatacaccttcggacagggtaccaagctggaaatcaagggagg |
| | | aggaggatctgggggcggtggttccggaggcggtggaagccaagtgcagctccaggaaa |
| | | gcggacccgggctggtcaagccgagcgaaaccctctcactgacttgtactgtgtccggagtg |
| | | tccctgcctgactatggagtgtcctggatccgacagccccccggaaagggtctggagtggatt |
| | | ggggtcatctggggctccgaaactacctactaccagagcagcctcaagagccgggtcaccat |
| | | ttcaaaggataactccaagaatcaagtgtccctgaagctgtcctcagtgacagccgcagacac |
| | | cgccgtgtactactgcgccaagcactactactacggaggctcctacgcaatggactactggg |
| | | gacaaggcactttggtcactgtgtcaagcaccaccacccctgcgcctcggcctcctaccccg |
| | | gctcccactatcgcgagccagccgctgagcctgcggcctgaggcttgccgaccggccgctg |
| | | gcggcgccgtgcatactcggggcctcgactttgcctgtgacatctacatctgggcccccctgg |
| | | ccggaacgtgcggagtgctgctgctgtcgctggtcattaccctgtattgcaaacgcggaagga |
| | | agaagctgttgtacattttcaagcagcccttcatgcgcccggtgcaaactactcaggaggaag |
| | | atggctgttcctgtcggttccccgaagaggaagaaggcggctgcgagttgagggtcaagttct |
| | | cccggtccgccgatgctcccgcctaccaacaggggcagaaccagctttataacgaactgaac |
| | | ctgggcaggagggaggaatatgatgtgttggataagcgccggggccgggacccagaaatg |
| | | gggggaaagcccagaagaaagaaccctcaagagggactttacaacgaattgcagaaagac |
| | | aaaatggccgaggcctactccgagattgggatgaagggcgaaagacggagaggaaaggg |
| | | gcacgacgggctctaccagggactcagcaccgccaccaaagatacctacgacgccctgcat |
| | | atgcaggcgctgccgccgcgc |
| Full length | 824 | MALPVTALLLPLALLLHAARPEVQLQQSGPGLVKPSQTLSL |
| CD19-CD22 | | TCAISGDSMLSNSDTWNWIRQSPSRGLEWLGRTYHRSTWY |
| Dual CAR | | DDYASSVRGRVSINVDTSKNQYSLQLNAVTPEDTGVYYCA |
| amino acid | | RVRLQDGNSWSDAFDVWGQGTMVTVSSGGGGSQSALTQP |
| | | ASASGSPGQSVTISCTGTSSDVGGYNYVSWYQQHPGKAPK |
| | | LMIYDVSNRPSGVSNRFSGSKSGNTASLTISGLQAEDEADY |
| | | YCSSYTSSSTLYVFGTGTQLTVLTTTPAPRPPTPAPTIASQPL |
| | | SLRPEACRPAAGGAVHTRGLDFACDIYIWAPLAGTCGVLL |
| | | LSLVITLYCKRGRKKLLYIFKQPFMRPVQTTQEEDGCSCRF |
| | | PEEEEGGCELRVKFSRSADAPAYQQGQNQLYNELNLGRRE |
| | | EYDVLDKRRGRDPEMGGKPRRKNPQEGLYNELQKDKMA |
| | | EAYSEIGMKGERRRGKGHDGLYQGLSTATKDTYDALHMQ |
| | | ALPPRGSGATNFSLLKQAGDVEENPGPMALPVTALLLPLAL |
| | | LLHAARPEIVMTQSPATLSLSPGERATLSCRASQDISKYLN |
| | | WYQQKPGQAPRLLIYHTSRLHSGIPARFSGSGSGTDYTLTIS |
| | | SLOPEDFAVYFCQQGNTLPYTFGQGTKLEIKGGGGSGGGG |
| | | SGGGGSQVQLQESGPGLVKPSETLSLTCTVSGVSLPDYGVS |
| | | WIRQPPGKGLEWIGVIWGSETTYYQSSLKSRVTISKDNSKN |
| | | QVSLKLSSVTAADTAVYYCAKHYYYGGSYAMDYWGQGT |
| | | LVTVSSTTTPAPRPPTPAPTIASQPLSLRPEACRPAAGGAVH |
| | | TRGLDFACDIYIWAPLAGTCGVLLLSLVITLYCKRGRKKLL |
| | | YIFKQPFMRPVQTTQEEDGCSCRFPEEEEGGCELRVKFSRS |
| | | ADAPAYQQGQNQLYNELNLGRREEYDVLDKRRGRDPEM |
| | | GGKPRRKNPQEGLYNELQKDKMAEAYSEIGMKGERRRGK |
| | | GHDGLYQGLSTATKDTYDALHMQALPPR |
| CD22 CAR | 825 | MALPVTALLLPLALLLHAARPEVQLQQSGPGLVKPSQTLSL |
| (with P2A | | TCAISGDSMLSNSDTWNWIRQSPSRGLEWLGRTYHRSTWY |
| site) | | DDYASSVRGRVSINVDTSKNQYSLQLNAVTPEDTGVYYCA |
| | | RVRLQDGNSWSDAFDVWGQGTMVTVSSGGGGSQSALTQP |
| | | ASASGSPGQSVTISCTGTSSDVGGYNYVSWYQQHPGKAPK |
| | | LMIYDVSNRPSGVSNRFSGSKSGNTASLTISGLQAEDEADY |
| | | YCSSYTSSSTLYVFGTGTQLTVLTTTPAPRPPTPAPTIASQPL |
| | | SLRPEACRPAAGGAVHTRGLDFACDIYIWAPLAGTCGVLL |
| | | LSLVITLYCKRGRKKLLYIFKQPFMRPVQTTQEEDGCSCRF |
| | | PEEEEGGCELRVKFSRSADAPAYQQGQNQLYNELNLGRRE |
| | | EYDVLDKRRGRDPEMGGKPRRKNPQEGLYNELQKDKMA |
| | | EAYSEIGMKGERRRGKGHDGLYQGLSTATKDTYDALHMQ |
| | | ALPPRGSGATNFSLLKQAGDVEENPG |
| CD19 CAR | 826 | PMALPVTALLLPLALLLHAARPEIVMTQSPATLSLSPGERA |
| | | TLSCRASQDISKYLNWYQQKPGQAPRLLIYHTSRLHSGIPA |
| | | RFSGSGSGTDYTLTISSLQPEDFAVYFCQQGNTLPYTFGQG |
| | | TKLEIKGGGGSGGGGSGGGGSQVQLQESGPGLVKPSETLSL |
| | | TCTVSGVSLPDYGVSWIRQPPGKGLEWIGVIWGSETTYYQS |
| | | SLKSRVTISKDNSKNQVSLKLSSVTAADTAVYYCAKHYYY |
| | | GGSYAMDYWGQGTLVTVSSTTTPAPRPPTPAPTIASQPLSL |
| | | RPEACRPAAGGAVHTRGLDFACDIYIWAPLAGTCGVLLLS |
| | | LVITLYCKRGRKKLLYIFKQPFMRPVQTTQEEDGCSCRFPE |
| | | EEEGGCELRVKFSRSADAPAYQQGQNQLYNELNLGRREEY |
| | | DVLDKRRGRDPEMGGKPRRKNPQEGLYNELQKDKMAEA |
| | | YSEIGMKGERRRGKGHDGLYQGLSTATKDTYDALHMQAL |
| | | PPR |

CD22 and CD19 CDRs of a dual CAR of the disclosure (e.g., a dual CAR molecule comprising (i) a first CAR that binds to CD22 and (ii) a second CAR that binds to CD 19) are provided in Table 31.

TABLE 31
CD22 and CD19 CDR sequences

| | | |
|---|---|---|
| | SEQ ID | |
| Identifier | NO | Sequence |

CD22 CDRs

| | | |
|---|---|---|
| HCDR1 | 745 | SNSDTWN |
| (Kabat) | | |
| HCDR2 | 746 | RTYHRSTWYDDYASSVRG |
| (Kabat) | | |
| HCDR3 | 748 | VRLQDGNSWSDAFDV |
| (Kabat) | | |
| HCDR1 | 832 | GDSMLSNSD |
| (Chothia) | | |
| HCDR2 | 833 | YHRSTWY |
| (Chothia) | | |
| HCDR3 | 748 | VRLQDGNSWSDAFDV |
| (Chothia) | | |
| HCDR1 | 834 | GDSMLSNSDT |
| (IMGT) | | |
| HCDR2 | 835 | TYHRSTWYD |
| (IMGT) | | |
| HCDR3 | 836 | ARVRLQDGNSWSDAFDV |
| (IMGT) | | |
| LCDR1 | 95 | TGTSSDVGGYNYVS |
| (Kabat) | | |
| LCDR2 | 96 | DVSNRPS |
| (Kabat) | | |
| LCDR3 | 97 | SSYTSSSTLYV |
| (Kabat) | | |
| LCDR1 | 98 | TSSDVGGYNY |
| (Chothia) | | |
| LCDR2 | 99 | DVS |
| (Chothia) | | |
| LCDR3 | 100 | YTSSSTLY |
| (Chothia) | | |
| LCDR1 | 101 | SSDVGGYNY |
| (IMGT) | | |
| LCDR2 | 99 | DVS |
| (IMGT) | | |
| LCDR3 | 97 | SSYTSSSTLYV |
| (IMGT) | | |

CD19 CDRs

| | | |
|---|---|---|
| HCDR1 | 837 | GVSLPDYGVS |
| (Kabat) | | |
| HCDR2 | 838 | VIWGSETTYYSSSLKS |
| (Kabat) | 687 | VIWGSETTYYQSSLKS |
| | 839 | VIWGSETTYYNSSLKS |
| HCDR3 | 762 | HYYYGGSYAMDY |
| (Kabat) | | |
| LCDR1 | 763 | RASQDISKYLN |
| LCDR2 | 764 | HTSRLHS |
| LCDR3 | 300 | QQGNTLPYT |

Table 32 provides nucleotide and amino acid sequence for CD19 and CD22 binding domains of a dual CAR or a tandem CAR disclosed herein, e.g., a dual CAR or a tandem CAR comprising (i) a first CAR that binds to CD22 and (ii) a second CAR that binds to CD 19.

TABLE 32
CD19 and CD22 binding domains

| | SEQ ID | |
|---|---|---|
| Identifier | NO | Sequence |
| scFv | 840 | gaaattgtgatgacccagtcacccgccactcttagcctttcacccggtgagcgcgcaaccctgt |
| CAR19 in | | cttgcagagcctcccaagacatctcaaaataccttaattggtatcaacagaagcccggacaggc |
| c201, c203 | | tcctcgccttctgatctaccacaccagccggctccattctggaatccctgccaggttcagcggta |
| and tandem | | gcggatctgggaccgactacaccctcactatcagctcactgcagccagaggacttcgctgtcta |
| CARs c171, | | tttctgtcagcaagggaacaccctgccctacacctttggacagggcaccaagctcgagattaaa |
| c182, c188 | | ggtggaggtggcagcggaggaggtgggtccggcggtggaggaagccaggtccaactccaa |
| | | gaaagcggaccgggtcttgtgaagccatcagaaactctttcactgacttgtactgtgagcggag |
| | | tgtctctccccgattacggggtgtcttggatcagacagccaccggggaagggtctggaatggat |
| | | tggagtgatttggggctctgagactacttactaccaatcatccctcaagtcacgcgtcaccatctc |
| | | aaaggacaactctaagaatcaggtgtcactgaaactgtcatctgtgaccgcagccgacaccgc |
| | | cgtgtactattgcgctaagcattactattatggcgggagctacgcaatggattactggggacagg |
| | | gtactctggtcaccgtgtccagc |
| | 758 | EIVMTQSPATLSLSPGERATLSCRASQDISKYLNWYQQKPG |
| | | QAPRLLIYHTSRLHSGIPARFSGSGSGTDYTLTISSLQPEDFA |
| | | VYFCQQGNTLPYTFGQGTKLEIKGGGGSGGGGSGGGGSQV |
| | | QLQESGPGLVKPSETLSLTCTVSGVSLPDYGVSWIRQPPGKG |
| | | LEWIGVIWGSETTYYQSSLKSRVTISKDNSKNQVSLKLSSVT |
| | | AADTAVYYCAKHYYYGGSYAMDYWGQGTLVTVSS |
| scFv | 841 | gagattgtcatgactcagtccccggccacactctccctgtcacccggagaaagagcaaccctg |
| CAR19 in | | agctgcagggcgtcccaggacatctcgaagtacctgaactggtaccagcagaagcctggaca |
| c224 | | agcaccccgcctcctgatctaccacacctcgcggctgcattcgggaatccccgccagattctca |
| | | gggagcggatcaggaaccgactacaccctgactatctcgagcctgcaaccagaggatttcgc |
| | | cgtgtacttctgccagcaaggaaacaccctgccctacacctttggacagggaaccaagctcga |
| | | gattaaggggggtggtggatcgggagggggtggatcaggaggaggcggctcacaagtcca |
| | | gctgcaagaatccggtccgggacttgtgaagccgtccgaaaccctgtcactgacttgcactgtg |
| | | tccggggtgtcattgcccgactacggcgtgagctggattcggcagccccctggaaagggattg |
| | | gaatggatcggcgtgatctggggttcggaaactacctactatcagtcctcactgaagtcccgcg |
| | | tgaccatcagcaaggataattccaaaaaccaagtgtctctgaagctctccagcgtcactgccgc |
| | | cgatactgccgtgtactactgcgccaagcactactattacggcggttcgtacgccatggactact |
| | | ggggccaagggacactcgtgaccgtgtcatcc |
| | 758 | EIVMTQSPATLSLSPGERATLSCRASQDISKYLNWYQQKPG |
| | | QAPRLLIYHTSRLHSGIPARFSGSGSGTDYTLTISSLQPEDFA |
| | | VYFCQQGNTLPYTFGQGTKLEIKGGGGSGGGGSGGGGSQV |
| | | QLQESGPGLVKPSETLSLTCTVSGVSLPDYGVSWIRQPPGKG |
| | | LEWIGVIWGSETTYYQSSLKSRVTISKDNSKNQVSLKLSSVT |
| | | AADTAVYYCAKHYYYGGSYAMDYWGQGTLVTVSS |
| scFv | 842 | caagtccagctgcaagaatccggtccgggacttgtgaagccgtccgaaaccctgtcactgactt |
| CAR19 in | | gcactgtgtccggggtgtcattgcccgactacggcgtgagctggattcggcagccccctggaa |
| c227 | | agggattggaatggatcggcgtgatctggggttcggaaactacctactatcagtcctcactgaa |
| | | gtcccgcgtgaccatcagcaaggataattccaaaaaccaagtgtctctgaagctctccagcgtc |
| | | actgccgccgatactgccgtgtactactgcgccaagcactactattacggcggttcgtacgccat |
| | | ggactactggggacaaggcactcttgtgactgtgtcaagcggcggtggagggagcggtggg |
| | | ggcggttcaggaggaggcggatcagagatcgtgatgacccaatccccagccaccctgtccct |
| | | cagccctggagaaagagccaccctgagctgccgggcctcccaggatatcagcaagtacttga |
| | | actggtaccaacaaaagccggggcaggcgccccggctcctgatctaccacacctcgcgcctc |
| | | cactcaggtatccccgccagattctcagggagcggctccggtactgactacaccctgactatttc |
| | | ctcactgcagccagaggactttgccgtgtacttctgccagcagggaaacactctgccgtacacc |
| | | ttcgggcagggaacgaagcttgaaattaag |
| | 843 | QVQLQESGPGLVKPSETLSLTCTVSGVSLPDYGVSWIRQPPG |
| | | KGLEWIGVIWGSETTYYQSSLKSRVTISKDNSKNQVSLKLSS |
| | | VTAADTAVYYCAKHYYYGGSYAMDYWGQGTLVTVSSGG |
| | | GGSGGGGSGGGGSEIVMTQSPATLSLSPGERATLSCRASQDI |
| | | SKYLNWYQQKPGQAPRLLIYHTSRLHSGIPARFSGSGSGTD |
| | | YTLTISSLQPEDFAVYFCQQGNTLPYTFGQGTKLEIK |
| scFv | 844 | gagattgtgatgactcagagcccggcgaccctgtccctgtcccccggggagagagcaaccct |
| CAR19 in | | gtcgtgccgggcctcccaagacatctcaaagtacctcaattggtatcagcagaagccaggaca |
| c230 | | ggctccacggttgctgatctaccacacttcgagactgcactcaggaatccccgcgcggttttcc |
| | | ggttccggctccgggaccgactacaccctgaccatcagctcgctccagcctgaggatttcgca |
| | | gtgtacttctgtcagcaaggaaacacccttccatacaccttcggacagggtaccaagctggaaa |
| | | tcaagggaggaggaggatctgggggcggtggttccggaggcggtggaagccaagtgcagc |
| | | tccaggaaagcggacccgggctggtcaagccgagcgaaaccctctcactgacttgtactgtgt |
| | | ccggagtgtccctgcctgactatggagtgtcctggatccgacagccccccggaaagggtctgg |
| | | agtggattggggtcatctggggctccgaaactacctactaccagagcagcctcaagagccgg |
| | | gtcaccatttcaaaggataactccaagaatcaagtgtccctgaagctgtcctcagtgacagccg |
| | | cagacaccgccgtgtactactgcgccaagcactactactacggaggctcctacgcaatggact |
| | | actggggacaaggcactttggtcactgtgtcaagc |
| | 758 | EIVMTQSPATLSLSPGERATLSCRASQDISKYLNWYQQKPG |
| | | QAPRLLIYHTSRLHSGIPARFSGSGSGTDYTLTISSLQPEDFA |
| | | VYFCQQGNTLPYTFGQGTKLEIKGGGGSGGGGSGGGGSQV |
| | | QLQESGPGLVKPSETLSLTCTVSGVSLPDYGVSWIRQPPGKG |
| | | LEWIGVIWGSETTYYQSSLKSRVTISKDNSKNQVSLKLSSVT |
| | | AADTAVYYCAKHYYYGGSYAMDYWGQGTLVTVSS |
| ScFVCAR2 | 845 | gaagtgcagctgcagcagtcagggcctggcctggtcaagccgtcgcagaccctctccctgac |
| 2 in c201 | | atgcgccattagcggggactccatgctgagcaactcggacacctggaactggattcggcagtc |
| and c203 | | cccttcccggggactcgagtggctcggacgcacctaccatcggagcacttggtacgacgacta |
| | | cgcctcctccgtgagaggtcgcgtgtcgatcaacgtggatacctcgaagaaccagtatagcttg |
| | | caactgaacgccgtgacccctgaggataccggagtgtactattgtgcgagagtcaggctgcaa |
| | | gacggaaactcctggtccgacgcatttgatgtctggggacagggtactatggtcacggtgtcat |
| | | ctggaggcggaggatcgcaaagcgccctgactcagccggcttcggctagcggttcaccggg |
| | | gcagtccgtgactatctcctgcaccgggacttcctccgacgtgggaggctacaattacgtgtcct |
| | | ggtaccagcaacaccccggcaaagccccaaagctgatgatctacgacgtcagcaacagacc |
| | | cagcggagtgtccaaccggttcagcggctccaagtccggcaacaccgcctccctgaccatca |
| | | gcgggcttcaggccgaagatgaggcggattactactgctcctcgtacacctcaagctcaactct |
| | | gtacgtgttcggcaccggtactcagctcaccgtgctg |
| | 846 | EVQLQQSGPGLVKPSQTLSLTCAISGDSMLSNSDTWNWIRQ |
| | | SPSRGLEWLGRTYHRSTWYDDYASSVRGRVSINVDTSKNQ |
| | | YSLQLNAVTPEDTGVYYCARVRLQDGNSWSDAFDVWGQG |
| | | TMVTVSSGGGGSQSALTQPASASGSPGQSVTISCTGTSSDVG |
| | | GYNYVSWYQQHPGKAPKLMIYDVSNRPSGVSNRFSGSKSG |
| | | NTASLTISGLQAEDEADYYCSSYTSSSTLYVFGTGTQLTVL |
| ScFVCAR2 | 847 | gaagtgcagctgcagcagtcaggaccgggcctggtcaaaccttcgcagactctgtccctgact |
| 2 in 230 | | tgcgctataagcggggactccatgctgagcaattcggacacttggaactggattcgccaaagc |
| | | cccagccggggtctggaatggctgggaaggacctaccatcgctctacttggtacgacgactac |
| | | gccagctccgtgcgaggacgcgtgtccatcaacgtggacacctccaagaaccagtactcgctt |
| | | caactcaacgcagtgacccctgaagataccggagtctactattgcgcccgcgtgcggctccag |
| | | gacgggaactcctggtcggacgctttcgatgtctggggacagggcactatggtcaccgtcagc |
| | | tccggcggcggcggtagccaatcggcgctgacacagccggcttccgcctcgggatcgcctg |
| | | gacagtcggtgaccatctcgtgcactggaacctcctccgacgtgggcggctacaattatgtgtc |
| | | atggtaccagcagcacccgggaaaggcccctaagctgatgatctacgacgtgtccaatagacc |
| | | tagcggggtgtcaaacagattctccggatccaaatccggaaacactgcctccctgaccatttcc |
| | | ggactgcaggccgaggacgaagccgattactactgctcctcttacacctcctcatccaccctcta |
| | | cgtgtttgggactgggacccagctgaccgtcctc |
| | 846 | EVQLQQSGPGLVKPSQTLSLTCAISGDSMLSNSDTWNWIRQ |
| | | SPSRGLEWLGRTYHRSTWYDDYASSVRGRVSINVDTSKNQ |
| | | YSLQLNAVTPEDTGVYYCARVRLQDGNSWSDAFDVWGQG |
| | | TMVTVSSGGGGSQSALTQPASASGSPGQSVTISCTGTSSDVG |
| | | GYNYVSWYQQHPGKAPKLMIYDVSNRPSGVSNRFSGSKSG |
| | | NTASLTISGLQAEDEADYYCSSYTSSSTLYVFGTGTQLTVL |
| ScFVCAR2 | 848 | gaagtgcagcttcaacaatcaggaccaggactcgtcaaaccatcacagaccctctccctcacat |
| 2 in 171, | | gtgccatctccggggactccatgttgagcaattccgacacttggaattggattagacaaagccc |
| c182 | | gtcccggggtctggaatggttgggacgcacctaccaccggtctacttggtacgacgactacgc |
| | | gtcatccgtgcggggaagagtgtccatcaacgtggacacctccaagaaccagtacagcctgc |
| | | agcttaatgccgtgactcctgaggatacgggcgtctactactgcgcccgcgtccgcctgcaag |
| | | acgggaacagctggagcgatgcattcgatgtctggggccagggaactatggtcaccgtgtcgt |
| | | ctgggggcggtggatcgggtggcgggggttcggggggcggcggctctcagtccgctcttac |
| | | ccaaccggcctcagcctcggggagccccggccagagcgtgaccatttcctgcaccggcactt |
| | | catccgacgtgggcggctacaactacgtgtcctggtaccaacagcacccgggaaaggccccc |
| | | aagctcatgatctacgacgtgtccaacaggccctcgggagtgtccaaccggttctcgggttcga |
| | | aatcgggaaacacagccagcctgaccatcagcggactgcaggctgaagatgaagccgacta |
| | | ctactgctcctcctacacctcgtcatccacgctctacgtgttcggcactggaactcagctgactgt |
| | | gctg |
| | 753 | EVQLQQSGPGLVKPSQTLSLTCAISGDSMLSNSDTWNWIRQ |
| | | SPSRGLEWLGRTYHRSTWYDDYASSVRGRVSINVDTSKNQ |
| | | YSLQLNAVTPEDTGVYYCARVRLQDGNSWSDAFDVWGQG |
| | | TMVTVSSGGGGSGGGGSGGGGSQSALTQPASASGSPGQSV |
| | | TISCTGTSSDVGGYNYVSWYQQHPGKAPKLMIYDVSNRPSG |
| | | VSNRFSGSKSGNTASLTISGLQAEDEADYYCSSYTSSSTLYV |
| | | FGTGTQLTVL |
| ScFVCAR2 | 849 | cagtccgctcttacccaaccggcctcagcctcggggagccccggccagagcgtgaccatttcc |
| 2 in c188 | | tgcaccggcacttcatccgacgtgggcggctacaactacgtgtcctggtaccaacagcacccg |
| | | ggaaaggcccccaagctcatgatctacgacgtgtccaacaggccctcgggagtgtccaaccg |
| | | gttctcgggttcgaaatcgggaaacacagccagcctgaccatcagcggactgcaggctgaag |
| | | atgaagccgactactactgctcctcctacacctcgtcatccacgctctacgtgttcggcactgga |
| | | actcagctgactgtgctgggcggaggaggctccgaagtgcagcttcaacaatcaggaccagg |
| | | actcgtcaaaccatcacagaccctctccctcacatgtgccatctccggggactccatgttgagca |
| | | attccgacacttggaattggattagacaaagcccgtcccggggtctggaatggttgggacgca |
| | | cctaccaccggtctacttggtacgacgactacgcgtcatccgtgcggggaagagtgtccatcaa |
| | | cgtggacacctccaagaaccagtacagcctgcagcttaatgccgtgactcctgaggatacggg |
| | | cgtctactactgcgcccgcgtccgcctgcaagacgggaacagctggagcgatgcattcgatgt |
| | | ctggggccagggaactatggtcaccgtgtcgtct |
| | 850 | QSALTQPASASGSPGQSVTISCTGTSSDVGGYNYVSWYQQH |
| | | PGKAPKLMIYDVSNRPSGVSNRFSGSKSGNTASLTISGLQAE |
| | | DEADYYCSSYTSSSTLYVFGTGTQLTVLGGGGSEVQLQQSG |
| | | PGLVKPSQTLSLTCAISGDSMLSNSDTWNWIRQSPSRGLEW |
| | | LGRTYHRSTWYDDYASSVRGRVSINVDTSKNQYSLQLNAV |
| | | TPEDTGVYYCARVRLQDGNSWSDAFDVWGQGTMVTVSS |
| ScFVCAR2 | 851 | cagtccgctctgactcagccggcctcagcttcggggtcccctggtcaaagcgtcactatttcctg |
| 2 in c224 | | taccggaacctcatcagacgtgggcggctacaattacgtgtcctggtaccaacagcaccccgg |
| | | aaaggctcctaagcttatgatctacgacgtgtccaaccggccgtcaggagtgtccaacagattct |
| | | ccggctccaagagcggaaacactgccagcttgaccattagcggcttgcaggccgaggacga |
| | | agccgactactactgctctagctacacatcctcgtctaccctctacgtgtttggaacggggaccc |
| | | agctgactgtgctcgggggtggaggatcagaggtgcaactccagcagtccggtcctggcctc |
| | | gtgaaaccgtcccaaaccctgtccctgacttgcgccatctcgggcgactccatgctgtccaattc |
| | | cgacacctggaactggattagacaatcgcctagccggggactcgaatggctgggccggacct |
| | | accaccggtccacgtggtatgacgactacgcaagctccgtccggggaagggtgtccattaacg |
| | | tcgatacctccaagaaccagtacagccttcagctgaacgctgtgacccccgaggataccggcg |
| | | tctactactgtgcaagagtgcgattgcaggatggaaactcgtggtcggacgcattcgatgtctg |
| | | gggacagggaactatggtgaccgtgtcctcg |
| | 850 | QSALTQPASASGSPGQSVTISCTGTSSDVGGYNYVSWYQQH |
| | | PGKAPKLMIYDVSNRPSGVSNRFSGSKSGNTASLTISGLQAE |
| | | DEADYYCSSYTSSSTLYVFGTGTQLTVLGGGGSEVQLQQSG |
| | | PGLVKPSQTLSLTCAISGDSMLSNSDTWNWIRQSPSRGLEW |
| | | LGRTYHRSTWYDDYASSVRGRVSINVDTSKNQYSLQLNAV |
| | | TPEDTGVYYCARVRLQDGNSWSDAFDVWGQGTMVTVSS |
| ScFVCAR2 | 852 | cagtccgctctgactcagccggcctcagcttcggggtcccctggtcaaagcgtcactatttcctg |
| 2 in c227 | | taccggaacctcatcagacgtgggcggctacaattacgtgtcctggtaccaacagcaccccgg |
| | | aaaggctcctaagcttatgatctacgacgtgtccaaccggccgtcaggagtgtccaacagattct |
| | | ccggctccaagagcggaaacactgccagcttgaccattagcggcttgcaggccgaggacga |
| | | agccgactactactgctctagctacacatcctcgtctaccctctacgtgtttggaacggggaccc |
| | | agctgactgtgctcgggggtggaggatcagaggtgcaactccagcagtccggtcctggcctc |
| | | gtgaaaccgtcccaaaccctgtccctgacttgcgccatctcgggcgactccatgctgtccaattc |
| | | cgacacctggaactggattagacaatcgcctagccggggactcgaatggctgggccggacct |
| | | accaccggtccacgtggtatgacgactacgcaagctccgtccggggaagggtgtccattaacg |
| | | tcgatacctccaagaaccagtacagccttcagctgaacgctgtgacccccgaggataccggcg |
| | | tctactactgtgcaagagtgcgattgcaggatggaaactcgtggtcggacgcattcgatgtctg |
| | | gggacagggaactatggtcactgtgtcctcc |
| | 850 | QSALTQPASASGSPGQSVTISCTGTSSDVGGYNYVSWYQQH |
| | | PGKAPKLMIYDVSNRPSGVSNRFSGSKSGNTASLTISGLQAE |
| | | DEADYYCSSYTSSSTLYVFGTGTQLTVLGGGGSEVQLQQSG |
| | | PGLVKPSQTLSLTCAISGDSMLSNSDTWNWIRQSPSRGLEW |
| | | LGRTYHRSTWYDDYASSVRGRVSINVDTSKNQYSLQLNAV |
| | | TPEDTGVYYCARVRLQDGNSWSDAFDVWGQGTMVTVSS |

Table 33 provides nucleotide and amino acid sequences for additional CAR components, e.g., signal peptide, linkers and P2A sites, that can be used in a CAR molecule, e.g., a dual CAR molecule described herein (for example, a dual CAR molecule comprising (i) a first CAR that binds to CD22 and (ii) a second CAR that binds to CD19).

TABLE 33
Additional CAR components

| | SEQ | |
|---|---|---|
| Identifier | ID NO | Sequence |
| Signal peptide for | 853 | atggccctccctgtcaccgccctgctgcttccgctggctcttctgctccacgccgctcggc |
| CAR22 in c201, | | cc |
| c203, and tandem | 2 | MALPVTALLLPLALLLHAARP |
| CARs c171, | | |
| c182, c188 | | |
| Signal peptide | 854 | atggccctgcccgtgactgcgctcctgcttccgttggccctgctcctgcatgccgccagac |
| in tandem CARs | | ct |
| c224, c227 | 2 | MALPVTALLLPLALLLHAARP |
| Signal peptide | 855 | atggccttaccagtgaccgccttgctcctgccgctggccttgctgctccacgccgccagg |
| CAR19 in c201 | | ccg |
| and c203 | 2 | MALPVTALLLPLALLLHAARP |
| Signal peptide | 856 | atggcacttcccgtcaccgccctgctgctcccactcgccctccttctgcacgccgcccgcc |
| CAR22 in c230 | | cc |
| | 2 | MALPVTALLLPLALLLHAARP |
| Signal peptide | 857 | atggccctgccagtgaccgcgctcctgctgcccctggctctgctgcttcacgcggcccgg |
| CAR19 in c230 | | cct |
| | 2 | MALPVTALLLPLALLLHAARP |
| CD8 hinge and | 858 | accactaccccagcaccgaggccacccaccccggctcctaccatcgcctcccagcctct |
| transmembrane | | gtccctgcgtccggaggcatgtagacccgcagctggtggggccgtgcatacccggggt |
| CAR22 in c201 | | cttgacttcgcctgcgatatctacatttgggcccctctggctggtacttgcggggtcctgctg |
| and c203, and in | | ctttcactcgtgatcactctttactgt |
| tandem CARs | 202 | TTTPAPRPPTPAPTIASQPLSLRPEACRPAAGGAVHTRGL |
| c171, c182, c188, | | DFACDIYIWAPLAGTCGVLLLSLVITLYC |
| c224, c227 | | |
| CD8 hinge and | 859 | actaccaccccggccccgcggccccctacaccggcaccgactattgccagccagcctct |
| transmembrane | | ctcgctgcggccggaggcctgccgcccagccgccggcggagccgtgcacacccgcg |
| CAR22 | | gtctggacttcgcgtgcgatatctacatctgggctccgctggccgggacttgtggcgtgct |
| In c230 | | gctgctgtctctggtcatcacactgtactgc |
| | 202 | TTTPAPRPPTPAPTIASQPLSLRPEACRPAAGGAVHTRGL |
| | | DFACDIYIWAPLAGTCGVLLLSLVITLYC |
| CD8 hinge and | 860 | accacgacgccagcgccgcgaccaccaacaccggcgcccaccatcgcgtcgcagccc |
| transmembrane | | ctgtccctgcgcccagaggcgtgccggccagcggcggggggcgcagtgcacacgag |
| CAR19 in c201 | | ggggctggacttcgcctgtgatatctacatctgggcgcccttggccgggacttgtggggtc |
| and c203 | | cttctcctgtcactggttatcaccctttactgc |
| | 202 | TTTPAPRPPTPAPTIASQPLSLRPEACRPAAGGAVHTRGL |
| | | DFACDIYIWAPLAGTCGVLLLSLVITLYC |
| CD8 hinge and | 861 | accaccacccctgcgcctcggcctcctaccccggctcccactatcgcgagccagccgct |
| transmembrane | | gagcctgcggcctgaggcttgccgaccggccgctggcggcgccgtgcatactcgggg |
| CAR19 | | cctcgactttgcctgtgacatctacatctgggcccccctggccggaacgtgcggagtgct |
| In c230 | | gctgctgtcgctggtcattaccctgtattgc |
| | 202 | TTTPAPRPPTPAPTIASQPLSLRPEACRPAAGGAVHTRGL |
| | | DFACDIYIWAPLAGTCGVLLLSLVITLYC |
| 4-1BB | 862 | aagcgcggtcggaagaagctgctgtacatctttaagcaacccttcatgaggcctgtgcag |
| CAR22 in c201 | | actactcaagaggaggacggctgttcatgccggttcccagaggaggaggaaggcggct |
| and c203, and | | gcgaactg |
| tandem CARs | 14 | KRGRKKLLYIFKQPFMRPVQTTQEEDGCSCRFPEEEEGG |
| c171, c182, c188, | | CEL |
| c224, c227 | | |
| 4-1BB | 15 | aaacggggcagaaagaaactcctgtatatattcaaacaaccatttatgagaccagtacaaa |
| CAR19 in c201 | | ctactcaagaggaagatggctgtagctgccgatttccagaagaagaagaaggaggatgt |
| and c203 | | gaactg |
| | 14 | KRGRKKLLYIFKQPFMRPVQTTQEEDGCSCRFPEEEEGG |
| | | CEL |
| 4-1BB | 863 | aagcgcggaagaaagaagctgctctacatcttcaagcaacccttcatgcggcctgtgcag |
| CAR22 in c230 | | accacccaggaagaggatggctgctcctgccggttcccggaggaagaagagggcgga |
| | | tgcgaactg |
| | 14 | KRGRKKLLYIFKQPFMRPVQTTQEEDGCSCRFPEEEEGG |
| | | CEL |
| 4-1BB | 864 | aaacgcggaaggaagaagctgttgtacattttcaagcagcccttcatgcgcccggtgcaa |
| CAR19 in c230 | | actactcaggaggaagatggctgttcctgtcggttccccgaagaggaagaaggcggctg |
| | | cgagttg |
| | 14 | KRGRKKLLYIFKQPFMRPVQTTQEEDGCSCRFPEEEEGG |
| | | CEL |
| CD3zeta | 865 | cgcgtgaaattcagccgcagcgcagatgctccagcctaccagcaggggcagaaccagc |
| CAR22 in c201 | | tctacaacgaactcaatcttggtcggagagaggagtacgacgtgctggacaagcggaga |
| and c203, and | | ggacgggacccagaaatgggcgggaagccgcgcagaaagaatccccaagagggcct |
| tandem CARs | | gtacaacgagctccaaaaggataagatggcagaagcctatagcgagattggtatgaaag |
| 171, c182, c188, | | gggaacgcagaagaggcaaaggccacgacggactgtaccagggactcagcaccgcc |
| c224, c227 | | accaaggacacctatgacgctcttcacatgcaggccctgccgcctcgg |
| | 20 | RVKFSRSADAPAYQQGQNQLYNELNLGRREEYDVLDKR |
| | | RGRDPEMGGKPRRKNPQEGLYNELQKDKMAEAYSEIG |
| | | MKGERRRGKGHDGLYQGLSTATKDTYDALHMQALPPR |
| CD3zeta | 21 | agagtgaagttcagcaggagcgcagacgcccccgcgtaccagcagggccagaaccag |
| CAR19 in c201 | | ctctataacgagctcaatctaggacgaagagaggagtacgatgttttggacaagagacgt |
| and c203 | | ggccgggaccctgagatggggggaaagccgagaaggaagaaccctcaggaaggcct |
| | | gtacaatgaactgcagaaagataagatggcggaggcctacagtgagattgggatgaaag |
| | | gcgagcgccggaggggcaaggggcacgatggcctttaccagggtctcagtacagcca |
| | | ccaaggacacctacgacgcccttcacatgcaggccctgccccctcgc |
| | 20 | RVKFSRSADAPAYQQGQNQLYNELNLGRREEYDVLDKR |
| | | RGRDPEMGGKPRRKNPQEGLYNELQKDKMAEAYSEIG |
| | | MKGERRRGKGHDGLYQGLSTATKDTYDALHMQALPPR |
| CD3zeta | 866 | cgcgtgaagttcagccgaagcgccgacgccccggcctaccagcagggccagaaccaa |
| CAR22 in c230 | | ctgtacaacgaactcaacctgggtcggagagaagagtacgacgtgctggacaaaagac |
| | | gcggcagggaccccgagatgggcggaaagcctcgccgcaagaacccgcaggaggg |
| | | cctctacaacgagctgcagaaggacaagatggccgaagcctactcagagatcggcatg |
| | | aagggggagcggaggcgcgggaagggccacgacggtttgtaccaaggactttccact |
| | | gcgaccaaggacacctacgatgccctccatatgcaagccctgccgccccgg |
| | 20 | RVKFSRSADAPAYQQGQNQLYNELNLGRREEYDVLDKR |
| | | RGRDPEMGGKPRRKNPQEGLYNELQKDKMAEAYSEIG |
| | | MKGERRRGKGHDGLYQGLSTATKDTYDALHMQALPPR |
| CD3zeta | 867 | agggtcaagttctcccggtccgccgatgctcccgcctaccaacaggggcagaaccagct |
| CAR19 in c230 | | ttataacgaactgaacctgggcaggagggaggaatatgatgtgttggataagcgccggg |
| | | gccgggacccagaaatggggggaaagcccagaagaaagaaccctcaagagggacttt |
| | | acaacgaattgcagaaagacaaaatggccgaggcctactccgagattgggatgaaggg |
| | | cgaaagacggagaggaaaggggcacgacgggctctaccagggactcagcaccgcca |
| | | ccaaagatacctacgacgccctgcatatgcaggcgctgccgccgcgc |
| | 20 | RVKFSRSADAPAYQQGQNQLYNELNLGRREEYDVLDKR |
| | | RGRDPEMGGKPRRKNPQEGLYNELQKDKMAEAYSEIG |
| | | MKGERRRGKGHDGLYQGLSTATKDTYDALHMQALPPR |
| Linker between | 868 | ttggcagaagccgccgcgaaa |
| scFVs in c171 | 869 | LAEAAAK |
| Linker between | 870 | ggtggaggtggcagcggaggaggtgggtccggcggtggaggaagc |
| scFVs in | | |
| c182, c188 | 104 | GGGGSGGGGSGGGGS |
| Linker between | 871 | ggcggaggcgggagcggaggaggaggctctggcggaggaggaagc |
| scFVs in c224 | 104 | GGGGSGGGGSGGGGS |
| Linker between | 872 | ggcggtggaggctcgggggggggcggctcaggaggaggcggctca |
| scFVs in c227 | 104 | GGGGSGGGGSGGGGS |
| P2A in c201, | 873 | ggaagcggagctactaacttcagcctgctgaagcaggctggagacgtggaggagaacc |
| c203 | | ctggacct |
| | 874 | GSGATNFSLLKQAGDVEENPGP |
| P2A in c230 | 875 | ggttccggagctaccaacttctcgctgttgaagcaggccggagatgtcgaggaaaaccc |
| | | gggacct |
| | 874 | GSGATNFSLLKQAGDVEENPGP |
| Gly4Ser linker | 876 | Ggtggaggtggcagc |
| | 877 | GGGGS |

In some embodiments, the CAR-expressing immune effector cell described herein can further comprise a second CAR, e.g., a second CAR that includes a different antigen binding domain, e.g., to the same target (e.g., a target described above) or a different target. In some embodiments, the second CAR includes an antigen binding domain to a target expressed on the same cancer cell type as the target of the first CAR. In some embodiments, the CAR-expressing immune effector cell comprises a first CAR that targets a first antigen and includes an intracellular signaling domain having a costimulatory signaling domain but not a primary signaling domain, and a second CAR that targets a second, different, antigen and includes an intracellular signaling domain having a primary signaling domain but not a costimulatory signaling domain. While not wishing to be bound by theory, placement of a costimulatory signaling domain, e.g., 4-1BB, CD28, CD27, or OX-40, onto the first CAR, and the primary signaling domain, e.g., CD3 zeta, on the second CAR can limit the CAR activity to cells where both targets are expressed. In some embodiments, the CAR expressing immune effector cell comprises a first CAR that includes an antigen binding domain that targets, e.g., a target described above, a transmembrane domain and a costimulatory domain and a second CAR that targets an antigen other than antigen targeted by the first CAR (e.g., an antigen expressed on the same cancer cell type as the first target) and includes an antigen binding domain, a transmembrane domain and a primary signaling domain. In another embodiment, the CAR expressing immune effector cell comprises a first CAR that includes an antigen binding domain that targets, e.g., a target described above, a transmembrane domain and a primary signaling domain and a second CAR that targets an antigen other than antigen targeted by the first CAR (e.g., an antigen expressed on the same cancer cell type as the first target) and includes an antigen binding domain to the antigen, a transmembrane domain and a costimulatory signaling domain.

In some embodiments, the CAR-expressing immune effector cell comprises a CAR described herein, e.g., a CAR to a target described above, and an inhibitory CAR. In some embodiments, the inhibitory CAR comprises an antigen binding domain that binds an antigen found on normal cells but not cancer cells, e.g., normal cells that also express the target. In some embodiments, the inhibitory CAR comprises the antigen binding domain, a transmembrane domain and an intracellular domain of an inhibitory molecule. For example, the intracellular domain of the inhibitory CAR can be an intracellular domain of PD1, PD-L1, CTLA-4, TIM-3, CEACAM (e.g., CEACAM-1, CEACAM-3 and/or CEACAM-5), LAG-3, VISTA, BTLA, TIGIT, LAIR1, CD160, 2B4, or TGFR beta.

In some embodiments, an immune effector cell (e.g., T cell, NK cell) comprises a first CAR comprising an antigen binding domain that binds to a tumor antigen as described herein, and a second CAR comprising a PD1 extracellular domain or a fragment thereof.

In some embodiments, the cell further comprises an inhibitory molecule as described above.

In some embodiments, the second CAR in the cell is an inhibitory CAR, wherein the inhibitory CAR comprises an antigen binding domain, a transmembrane domain, and an intracellular domain of an inhibitory molecule. The inhibitory molecule can be chosen from one or more of: PD1, PD-L1, CTLA-4, TIM-3, LAG-3, VISTA, BTLA, TIGIT, LAIR1, CD160, 2B4, TGFR beta, CEACAM-1, CEACAM-3, and CEACAM-5. In some embodiments, the second CAR molecule comprises the extracellular domain of PD1 or a fragment thereof.

In embodiments, the second CAR molecule in the cell further comprises an intracellular signaling domain comprising a primary signaling domain and/or an intracellular signaling domain.

In other embodiments, the intracellular signaling domain in the cell comprises a primary signaling domain comprising the functional domain of CD3 zeta and a costimulatory signaling domain comprising the functional domain of 4-1BB.

In some embodiments, the antigen binding domain of the first CAR molecule comprises a scFv and the antigen binding domain of the second CAR molecule does not comprise a scFv. For example, the antigen binding domain of the first CAR molecule comprises a scFv and the antigen binding domain of the second CAR molecule comprises a camelid VHH domain.

Conformation of CARs

In the embodiments contemplated herein, it is appreciated that the conformation of one or more CARs could be modulated by the vectors described herein above. Accordingly, these conformations are described below in relation to the CAR-expressing cell.

Split CAR

In some embodiments, the CAR-expressing cell uses a split CAR. The split CAR approach is described in more detail in publications WO2014/055442 and WO2014/055657. Briefly, a split CAR system comprises a cell expressing a first CAR having a first antigen binding domain and a costimulatory domain (e.g., 41B), and the cell also expresses a second CAR having a second antigen binding domain and an intracellular signaling domain (e.g., CD3 zeta). When the cell encounters the first antigen, the costimulatory domain is activated, and the cell proliferates. When the cell encounters the second antigen, the intracellular signaling domain is activated and cell-killing activity begins. Thus, the CAR-expressing cell is only fully activated in the presence of both antigens.

Multiple CAR

In some aspects, the CAR-expressing cell described herein can further comprise a second CAR, e.g., a second CAR that includes a different antigen binding domain, e.g., to the same target or a different target (e.g., a target other than a cancer associated antigen described herein or a different cancer associated antigen described herein). In some embodiments, the second CAR includes an antigen binding domain to a target expressed the same cancer cell type as the cancer associated antigen. In some embodiments, the CAR-expressing cell comprises a first CAR that targets a first antigen and includes an intracellular signaling domain having a costimulatory signaling domain but not a primary signaling domain, and a second CAR that targets a second, different, antigen and includes an intracellular signaling domain having a primary signaling domain but not a costimulatory signaling domain. While not wishing to be bound by theory, placement of a costimulatory signaling domain, e.g., 4-1BB, CD28, CD27 or OX-40, onto the first CAR, and the primary signaling domain, e.g., CD3 zeta, on the second CAR can limit the CAR activity to cells where both targets are expressed. In some embodiments, the CAR expressing cell comprises a first cancer associated antigen CAR that includes an antigen binding domain that binds a target antigen described herein, a transmembrane domain and a costimulatory domain and a second CAR that targets a different target antigen (e.g., an antigen expressed on that same cancer cell type as the first target antigen) and includes an antigen binding domain, a transmembrane domain and a primary signaling domain. In another embodiment, the CAR expressing cell comprises a first CAR that includes an antigen binding domain that binds a target antigen described herein, a transmembrane domain and a primary signaling domain and a second CAR that targets an antigen other than the first target antigen (e.g., an antigen expressed on the same cancer cell type as the first target antigen) and includes an antigen binding domain to the antigen, a transmembrane domain and a costimulatory signaling domain.

In some embodiments, the claimed invention comprises a first and second CAR, wherein the antigen binding domain of one of said first CAR said second CAR does not comprise a variable light domain and a variable heavy domain. In some embodiments, the antigen binding domain of one of said first CAR said second CAR is an scFv, and the other is not an scFv. In some embodiments, the antigen binding domain of one of said first CAR said second CAR comprises a single VH domain, e.g., a camelid, shark, or lamprey single VH domain, or a single VH domain derived from a human or mouse sequence. In some embodiments, the antigen binding domain of one of said first CAR said second CAR comprises a nanobody. In some embodiments, the antigen binding domain of one of said first CAR said second CAR comprises a camelid VHH domain.

Once the methods described herein are performed, various assays can be used to evaluate the activity of, for e.g., the ability to expand T cells following antigen stimulation, sustain T cell expansion in the absence of re-stimulation, and anti-cancer activities in appropriate in vitro and animal models. Assays to evaluate the effects of a CAR of the present invention are known to those of skill in the art and generally described below.

Western blot analysis of CAR expression in primary T cells can be used to detect the presence of monomers and dimers. See, e.g., Milone et al., Molecular Therapy 17(8): 1453-1464 (2009). Very briefly, T cells (1:1 mixture of CD4⁺ and CD8⁺ T cells) expressing the CARs are expanded in vitro for more than 10 days followed by lysis and SDS-PAGE under reducing conditions. CARs containing the full length TCR-ζ cytoplasmic domain and the endogenous TCR-ζ chain are detected by western blotting using an antibody to the TCR-ζ chain. The same T cell subsets are used for SDS-PAGE analysis under non-reducing conditions to permit evaluation of covalent dimer formation.

In vitro expansion of CAR⁺ T cells following antigen stimulation can be measured by flow cytometry.

Sustained CAR⁺ T cell expansion in the absence of re-stimulation can also be measured. See, e.g., Milone et al., Molecular Therapy 17(8): 1453-1464 (2009). Briefly, mean T cell volume (fl) is measured on day 8 of culture using a Coulter Multisizer III particle counter, a Nexcelom Cellometer Vision or Millipore Scepter, following stimulation with αCD3/αCD28 coated magnetic beads on day 0, and transduction with the indicated CAR on day 1.

Animal models can also be used to measure a CART activity. For example, xenograft model using human a cancer associated antigen described herein-specific CAR⁺ T cells to treat a primary human pre-B ALL in immunodeficient mice can be used. See, e.g., Milone et al., Molecular Therapy 17(8): 1453-1464 (2009).

Dose dependent CAR treatment response can be evaluated. See, e.g., Milone et al., Molecular Therapy 17(8): 1453-1464 (2009). For example, peripheral blood is obtained 35-70 days after establishing leukemia in mice injected on day 21 with CAR T cells, an equivalent number of mock-transduced T cells, or no T cells. Mice from each group are randomly bled for determination of peripheral blood a cancer associate antigen as described herein⁺ ALL blast counts and then killed on days 35 and 49. The remaining animals are evaluated on days 57 and 70.

Assessment of cell proliferation and cytokine production has been previously described, e.g., at Milone et al., Molecular Therapy 17(8): 1453-1464 (2009).

Cytotoxicity can be assessed by a standard 51Cr-release assay. See, e.g., Milone et al., Molecular Therapy 17(8): 1453-1464 (2009

Imaging technologies can be used to evaluate specific trafficking and proliferation of CARs in tumor-bearing animal models. Such assays have been described, for example, in Barrett et al., Human Gene Therapy 22:1575-1586 (2011).

Other assays, including those described in the Example section herein as well as those that are known in the art can also be used to evaluate the CARs described herein.

### Anti-CD28 Antibody Molecules

In some embodiments, the anti-CD28 antibody, e.g., an anti-CD28 antibody to be used in a multispecific binding molecule described herein, comprises at least one antigen-binding region, e.g., a variable region or an antigen-binding fragment thereof, from anti-CD28 (2) as described in Table 19. In some embodiments, the anti-CD28 antibody molecule comprises one or two variable regions from anti-CD28 (2), as described in Table 19. In some embodiments, the anti-CD28 antibody comprises a heavy chain variable region (VH) comprising a heavy chain complementarity determining region 1 (HCDR1), a HCDR2, and a HCDR3, and a light chain variable region (VL) comprising a light chain complementarity determining region 1 (LCDR1), a LCDR2, and a LCDR3, wherein the HCDR1, HCDR2, HCDR3, LCDR1, LCDR2, and LCDR3 comprise the amino acid sequences of SEQ ID NOs: 538, 539, 540, 530, 531, and 532, respectively; the HCDR1, HCDR2, HCDR3, LCDR1, LCDR2, and LCDR3 comprise the amino acid sequences of SEQ ID NOs: 541, 539, 540, 530, 531, and 532, respectively; the HCDR1, HCDR2, HCDR3, LCDR1, LCDR2, and LCDR3 comprise the amino acid sequences of SEQ ID NOs: 542, 543, 540, 533, 534, and 535, respectively; or the HCDR1, HCDR2, HCDR3, LCDR1, LCDR2, and LCDR3 comprise the amino acid sequences of SEQ ID NOs: 544, 545, 546, 536, 534, and 532, respectively.

In some embodiments, the anti-CD28 antibody molecule comprises a VH comprising the amino acid sequence of SEQ ID NO: 547 or 548, or a sequence having at least about 80%, 85%, 90%, 92%, 95%, 97%, 98%, or 99% sequence identity to SEQ ID NO: 547 or 548. In some embodiments, the anti-CD28 antibody comprises a VL comprising the amino acid sequence of SEQ ID NO: 537, or a sequence having at least about 80%, 85%, 90%, 92%, 95%, 97%, 98%, or 99% sequence identity thereto. In some embodiments, the anti-CD28 antibody comprises a VH and a VL comprising the amino acid sequences of SEQ ID NOs: 547 and 537, respectively, or a sequence having at least about 80%, 85%, 90%, 92%, 95%, 97%, 98%, or 99% sequence identity to any of the aforesaid sequences. In some embodiments, the anti-CD28 antibody comprises a VH and a VL comprising the amino acid sequences of SEQ ID NOs: 548 and 537, respectively, or a sequence having at least about 80%, 85%, 90%, 92%, 95%, 97%, 98%, or 99% sequence identity to any of the aforesaid sequences.

It is understood that an anti-CD28 antibody described herein can be used in the context of a multispecific binding molecule, e.g., with an additional binding domain, e.g., an anti-CD3 binding domain described herein. It is also understood that anti-CD28 antibody described herein can be used in other contexts, e.g., as a monospecific antibody.

Cell Activation Agents

In some embodiments, the cell activation agent is a T cell stimulating compound, an anti-idiotype antibody to a CAR antigen binding domain, and/or a tumor antigen. In some embodiments, the T-cell stimulating compound is IL-2, IL-15, anti-CD2 mAb, anti-CD3 mAb, anti-CD28 mAb, neo-antigen peptides, peptides from shared antigens such as TRP2, gp100, tumor cell lysate, CD19, CD20, CD22, ROR1, mesothelin, CD33/IL3Ra, c-Met, PSMA, Glycolipid F77, EGFRvIII, GD-2, NY-ESO-1 TCR, and/or MAGE A3 TCR. In some embodiments, the cell activation agent comprises a CD3/TCR complex and/or an agent that stimulates a costimulatory molecule and/or growth factor receptor, optionally, wherein the cell activation agent is a multispecific binding molecule comprising an agent that stimulates a CD3/TCR complex and an agent that stimulates a costimulatory molecule and/or growth factor receptor.

In some embodiments, the agent that stimulates a CD3/TCR complex is an agent that stimulates CD3. In some embodiments, the agent that stimulates CD3 comprises one or more of a CD3 or TCR antigen binding domain, such as but not limited to an anti-CD3 or anti-TCR antibody or an antibody fragment comprising one or more CDRs, heavy chain, and/or light chain thereof.

Anti-CD3 antibody sequences and methods of making such antibodies are known in the art. Non-limiting examples of anti-CD3 antibody sequences, along with the relevant CDR, heavy chain, and light chain sequences are provided in Table 19. In some embodiments, the anti-CD3 binding domain comprises a VH and a VL comprising the amino acid sequences of SEQ ID NOs: 437 and 427, respectively. In some embodiments, the anti-CD3 binding domain comprises a VH and a VL comprising the amino acid sequences of SEQ ID NOs: 456 and 445, respectively. In some embodiments, the anti-CD3 binding domain comprises a VH and a VL comprising the amino acid sequences of SEQ ID NOs: 457 and 446, respectively. In some embodiments, the anti-CD3 binding domain comprises a VH and a VL comprising the amino acid sequences of SEQ ID NOs: 475 and 467, respectively. In some embodiments, the anti-CD3 binding domain comprises a VH and a VL comprising the amino acid sequences of SEQ ID NOs: 476 and 468, respectively. In some embodiments, the anti-CD3 binding domain comprises a VH and a VL comprising the amino acid sequences of SEQ ID NOs: 494 and 484, respectively

Anti-TCR antibody sequences and methods of making such antibodies are known in the art. Non-limiting examples of anti-TCR antibody sequences, along with the relevant CDR, heavy chain, and light chain sequences are provided in Table 19.

In some embodiments, the agent that stimulates a costimulatory molecule and/or growth factor receptor is an agent that stimulates CD28, ICOS, CD27, CD25, 4-1BB, TL6RA, IL6RB, or CD2. In some embodiments, the agent that stimulates a costimulatory molecule and/or growth factor receptor comprises one or more of a CD28, ICOS, CD27, CD25, 4-1BB, IL6RB, and/or CD2 antigen binding domain, such as but not limited to an anti-CD28, anti-ICOS, anti-CD27, anti-CD25, anti-4-1BB, anti-TL6RA, anti-IL6RB, or anti-CD2 antibody or an antibody fragment comprising one or more CDRs, heavy chain, and/or light chain thereof.

Anti-CD28 antibody sequences and methods of making such antibodies are known in the art. Non-limiting examples of anti-CD28 antibody sequences, along with the relevant CDR, VH, VL, HC and LC sequences are provided in Table 19.

Anti-ICOS antibody sequences and methods of making such antibodies are known in the art. Non-limiting examples of anti-ICOS antibody sequences, along with the relevant CDR, VH, VL, and LC sequences are provided in Table 19.

Anti-CD27 antibody sequences and methods of making such antibodies are known in the art. Non-limiting examples of anti-CD27 antibody sequences, along with the relevant CDR, VH, and VL sequences are provided in Table 19.

Anti-CD25 antibody sequences and methods of making such antibodies are known in the art. Non-limiting examples of anti-CD25 antibody sequences, along with the relevant CDR, VH, VL, HC, and LC sequences are provided in Table 19.

Anti-4-1BB antibody sequences and methods of making such antibodies are known in the art. Non-limiting examples of anti-4-IBB antibody sequences, along with the relevant CDR, VH, and VL sequences are provided in Table 19.

Anti-IL6RA antibody sequences and methods of making such antibodies are known in the art. Non-limiting examples of IL6RA antibody sequences, along with the relevant CDR, VH, and VL sequences are provided in Table 19.

Anti-IL6RB antibody sequences and methods of making such antibodies are known in the art. Non-limiting examples of IL6RB antibody sequences, along with the relevant CDR, VH, and VL sequences are provided in Table 19.

Anti-CD2 antibody sequences and methods of making such antibodies are known in the art. Non-limiting examples of anti-CD2 antibody sequences, along with the relevant CDR, VH, VL, HC and LC sequences are provided in Table 19.

In some embodiments, an antibody molecule described herein comprises a CDR, VH, VL, HC, and/or LC disclosed in Table 19, or sequence having at least 70%, 75%, 80%, 85%, 90%, 95%, 96%, 97%, 98%, or 99% identity thereto.

TABLE 19
Exemplary antibody, CDR, heavy chain variable
region (VH), light chain variable region (VL),
heavy chain (HC), and light chain (LC),
sequences by target antigen

| SEQ ID NO | Description | Amino acid sequence |
|---|---|---|
| ANTI-CD3 (1) | | |
| SEQ ID NO: 420 | LCDR1 | SASSSVSYMN |
| (Combined) | | |
| SEQ ID NO: 421 | LCDR2 | DTSKLAS |
| (Combined) | | |
| SEQ ID NO: 422 | LCDR3 | QQWSSNPFT |
| (Combined) | | |
| SEQ ID NO: 420 | LCDR1 | SASSSVSYMN |
| (Kabat) | | |
| SEQ ID NO: 421 | LCDR2 | DTSKLAS |
| (Kabat) | | |
| SEQ ID NO: 422 | LCDR3 | QQWSSNPFT |
| (Kabat) | | |
| SEQ ID NO: 423 | LCDR1 | SSSVSY |
| (Chothia) | | |
| SEQ ID NO: 424 | LCDR2 | DTS |
| (Chothia) | | |
| SEQ ID NO: 425 | LCDR3 | WSSNPF |
| (Chothia) | | |
| SEQ ID NO: 426 | LCDR1 | SSVSY |
| (IMGT) | | |
| SEQ ID NO: 424 | LCDR2 | DTS |
| (IMGT) | | |
| SEQ ID NO: 422 | LCDR3 | QQWSSNPFT |
| (IMGT) | | |
| SEQ ID NO: 427 | VL | DIQMTQSPSSLSASVGDRVTITCSASSSVSYM |
| | | NWYQQTPGKAPKRWIYDTSKLASGVPSRFSG |
| | | SGSGTDYTFTISSLQPEDIATYYCQQWSSNPFT |
| | | FGQGTKLQIT |
| SEQ ID NO: 428 | HCDR1 | GYTFTRYTMH |
| (Combined) | | |
| SEQ ID NO: 429 | HCDR2 | YINPSRGYTNYNQKVKD |
| (Combined) | | |
| SEQ ID NO: 430 | HCDR3 | YYDDHYSLDY |
| (Combined) | | |
| SEQ ID NO: 431 | HCDR1 | RYTMH |
| (Kabat) | | |
| SEQ ID NO: 429 | HCDR2 | YINPSRGYTNYNQKVKD |
| (Kabat) | | |
| SEQ ID NO: 430 | HCDR3 | YYDDHYSLDY |
| (Kabat) | | |
| SEQ ID NO: 432 | HCDR1 | GYTFTRY |
| (Chothia) | | |
| SEQ ID NO: 433 | HCDR2 | NPSRGY |
| (Chothia) | | |
| SEQ ID NO: 430 | HCDR3 | YYDDHYSLDY |
| (Chothia) | | |
| SEQ ID NO: 434 | HCDR1 | GYTFTRYT |
| (IMGT) | | |
| SEQ ID NO: 435 | HCDR2 | INPSRGYT |
| (IMGT) | | |
| SEQ ID NO: 436 | HCDR3 | ARYYDDHYSLDY |
| (IMGT) | | |
| SEQ ID NO: 437 | VH | QVQLVQSGGGVVQPGRSLRLSCKASGYTFTR |
| | | YTMHWVRQAPGKGLEWIGYINPSRGYTNYN |
| | | QKVKDRFTISRDNSKNTAFLQMDSLRPEDTG |
| | | VYFCARYYDDHYSLDYWGQGTPVTVSS |
| ANTI-CD3 (2) | | |
| SEQ ID NO: 438 | LCDR1 | GSSTGAVTSGNYPN |
| (Combined) | | |
| SEQ ID NO: 439 | LCDR2 | GTKFLAP |
| (Combined) | | |
| SEQ ID NO: 440 | LCDR3 | VLWYSNRWV |
| (Combined) | | |
| SEQ ID NO: 438 | LCDR1 | GSSTGAVTSGNYPN |
| (Kabat) | | |
| SEQ ID NO: 439 | LCDR2 | GTKFLAP |
| (Kabat) | | |
| SEQ ID NO: 440 | LCDR3 | VLWYSNRWV |
| (Kabat) | | |
| SEQ ID NO: 441 | LCDR1 | STGAVTSGNY |
| (Chothia) | | |
| SEQ ID NO: 442 | LCDR2 | GTK |
| (Chothia) | | |
| SEQ ID NO: 443 | LCDR3 | WYSNRW |
| (Chothia) | | |
| SEQ ID NO: 444 | LCDR1 | TGAVTSGNY |
| (IMGT) | | |
| SEQ ID NO: 442 | LCDR2 | GTK |
| (IMGT) | | |
| SEQ ID NO: 440 | LCDR3 | VLWYSNRWV |
| (IMGT) | | |
| SEQ ID NO: 445 | VL | QTVVTQEPSLTVSPGGTVTLTCGSSTGAVTSG |
| | | NYPNWVQQKPGQAPRGLIGGTKFLAPGTPAR |
| | | FSGSLLGGKAALTLSGVQPEDEAEYYCVLWY |
| | | SNRWVFGGGTKLTVL |
| SEQ ID NO: 446 | VL | QTVVTQEPSLTVSPGGTVTLTCGSSTGAVTSG |
| | | NYPNWVQQKPGQAPRGLIGGTKFLAPGTPAR |
| | | FSGSLLGGKAALTLSGVQPEDEAEYYCVLWY |
| | | SNRWVFGCGTKLTVL |
| SEQ ID NO: 447 | HCDR1 | GFTFNKYAMN |
| (Combined) | | |
| SEQ ID NO: 448 | HCDR2 | RIRSKYNNYATYYADSVKD |
| (Combined) | | |
| SEQ ID NO: 449 | HCDR3 | HGNFGNSYISYWAY |
| (Combined) | | |
| SEQ ID NO: 450 | HCDR1 | KYAMN |
| (Kabat) | | |
| SEQ ID NO: 448 | HCDR2 | RIRSKYNNYATYYADSVKD |
| (Kabat) | | |
| SEQ ID NO: 449 | HCDR3 | HGNFGNSYISYWAY |
| (Kabat) | | |
| SEQ ID NO: 451 | HCDR1 | GFTFNKY |
| (Chothia) | | |
| SEQ ID NO: 452 | HCDR2 | RSKYNNYA |
| (Chothia) | | |
| SEQ ID NO: 449 | HCDR3 | HGNFGNSYISYWAY |
| (Chothia) | | |
| SEQ ID NO: 453 | HCDR1 | GFTFNKYA |
| (IMGT) | | |
| SEQ ID NO: 454 | HCDR2 | IRSKYNNYAT |
| (IMGT) | | |
| SEQ ID NO: 455 | HCDR3 | VRHGNFGNSYISYWAY |
| (IMGT) | | |
| SEQ ID NO: 456 | VH | EVQLVESGGGLVQPGGSLKLSCAASGFTFNK |
| | | YAMNWVRQAPGKGLEWVARIRSKYNNYAT |
| | | YYADSVKDRFTISRDDSKNTAYLQMNNLKTE |
| | | DTAVYYCVRHGNFGNSYISYWAYWGQGTLV |
| | | TVSS |
| SEQ ID NO: 457 | VH | EVQLVESGGGLVQPGGSLKLSCAASGFTFNK |
| | | YAMNWVRQAPGKCLEWVARIRSKYNNYAT |
| | | YYADSVKDRFTISRDDSKNTAYLQMNNLKTE |
| | | DTAVYYCVRHGNFGNSYISYWAYWGQGTLV |
| | | TVSS |
| ANTI-CD3 (3) | | |
| SEQ ID NO: 458 | LCDR1 | RSSTGAVTTSNYAN |
| (Combined) | | |
| SEQ ID NO: 459 | LCDR2 | GTNKRAP |
| (Combined) | | |
| SEQ ID NO: 460 | LCDR3 | ALWYSNLWV |
| (Combined) | | |
| SEQ ID NO: 458 | LCDR1 | RSSTGAVTTSNYAN |
| (Kabat) | | |
| SEQ ID NO: 459 | LCDR2 | GTNKRAP |
| (Kabat) | | |
| SEQ ID NO: 460 | LCDR3 | ALWYSNLWV |
| (Kabat) | | |
| SEQ ID NO: 461 | LCDR1 | STGAVTTSNY |
| (Chothia) | | |
| SEQ ID NO: 462 | LCDR2 | GTN |
| (Chothia) | | |
| SEQ ID NO: 463 | LCDR3 | WYSNLW |
| (Chothia) | | |
| SEQ ID NO: 464 | LCDR1 | TGAVTTSNY |
| (IMGT) | | |
| SEQ ID NO: 465 | LCDR2 | GGT |
| (IMGT) | | |
| SEQ ID NO: 466 | LCDR3 | ALWYSNL |
| (IMGT) | | |
| SEQ ID NO: 467 | VL | QAVVTQEPSLTVSPGGTVTLTCRSSTGAVTTS |
| | | NYANWVQQKPGQAPRGLIGGTNKRAPWTPA |
| | | RFSGSLLGDKAALTLSGAQPEDEAEYFCALW |
| | | YSNLWVFGGGTKLTVL |
| SEQ ID NO: 468 | VL | QAVVTQEPSLTVSPGGTVTLTCRSSTGAVTTS |
| | | NYANWVQQKPGQAPRGLIGGTNKRAPWTPA |
| | | RFSGSLLGDKAALTLSGAQPEDEAEYFCALW |
| | | YSNLWVFGCGTKLTVL |
| SEQ ID NO: 469 | HCDR1 | GFTFNTYAMN |
| (Combined) | | |
| SEQ ID NO: 448 | HCDR2 | RIRSKYNNYATYYADSVKD |
| (Combined) | | |
| SEQ ID NO: 470 | HCDR3 | HGNFGNSYVSWFAY |
| (Combined) | | |
| SEQ ID NO: 471 | HCDR1 | TYAMN |
| (Kabat) | | |
| SEQ ID NO: 448 | HCDR2 | RIRSKYNNYATYYADSVKD |
| (Kabat) | | |
| SEQ ID NO: 470 | HCDR3 | HGNFGNSYVSWFAY |
| (Kabat) | | |
| SEQ ID NO: 472 | HCDR1 | GFTFNTY |
| (Chothia) | | |
| SEQ ID NO: 452 | HCDR2 | RSKYNNYA |
| (Chothia) | | |
| SEQ ID NO: 470 | HCDR3 | HGNFGNSYVSWFAY |
| (Chothia) | | |
| SEQ ID NO: 473 | HCDR1 | GFTFNTYA |
| (IMGT) | | |
| SEQ ID NO: 454 | HCDR2 | IRSKYNNYAT |
| (IMGT) | | |
| SEQ ID NO: 474 | HCDR3 | VRHGNFGNSYVSWFAY |
| (IMGT) | | |
| SEQ ID NO: 475 | VH | EVQLVESGGGLVQPGGSLKLSCAASGFTFNT |
| | | YAMNWVRQASGKGLEWVGRIRSKYNNYAT |
| | | YYADSVKDRFTISRDDSKSTLYLQMNSLKTE |
| | | DTAVYYCVRHGNFGNSYVSWFAYWGQGTL |
| | | VTVSS |
| SEQ ID NO: 476 | VH | EVQLVESGGGLVQPGGSLKLSCAASGFTFNT |
| | | YAMNWVRQASGKCLEWVGRIRSKYNNYAT |
| | | YYADSVKDRFTISRDDSKSTLYLQMNSLKTE |
| | | DTAVYYCVRHGNFGNSYVSWFAYWGQGTL |
| | | VTVSS |
| ANTI-CD3 (4) | | |
| SEQ ID NO: 477 | LCDR1 | RSSQSLVRSEGTTYFN |
| (Combined) | | |
| SEQ ID NO: 478 | LCDR2 | RVSNRFS |
| (Combined) | | |
| SEQ ID NO: 479 | LCDR3 | LQSSHFPWT |
| (Combined) | | |
| SEQ ID NO: 477 | LCDR1 | RSSQSLVRSEGTTYFN |
| (Kabat) | | |
| SEQ ID NO: 478 | LCDR2 | RVSNRFS |
| (Kabat) | | |
| SEQ ID NO: 479 | LCDR3 | LQSSHFPWT |
| (Kabat) | | |
| SEQ ID NO: 480 | LCDR1 | SQSLVRSEGTTY |
| (Chothia) | | |
| SEQ ID NO: 481 | LCDR2 | RVS |
| (Chothia) | | |
| SEQ ID NO: 482 | LCDR3 | SSHFPW |
| (Chothia) | | |
| SEQ ID NO: 483 | LCDR1 | QSLVRSEGTTY |
| (IMGT) | | |
| SEQ ID NO: 481 | LCDR2 | RVS |
| (IMGT) | | |
| SEQ ID NO: 479 | LCDR3 | LQSSHFPWT |
| (IMGT) | | |
| SEQ ID NO: 484 | VL | DILVTQTPVSLPVSLGGHVSISCRSSQSLVRSE |
| | | GTTYFNWYLQKPGQSPQLLIYRVSNRFSGVP |
| | | DRFSGSGSGTDFTLKISRVEPEDLGVYYCLQS |
| | | SHFPWTFGGGTKLELK |
| SEQ ID NO: 485 | HCDR1 | GFTFSKQGMH |
| (Combined) | | |
| SEQ ID NO: 486 | HCDR2 | MIYYDSSKMYYADTVKG |
| (Combined) | | |
| SEQ ID NO: 487 | HCDR3 | FWWDLDFDH |
| (Combined) | | |
| SEQ ID NO: 488 | HCDR1 | KQGMH |
| (Kabat) | | |
| SEQ ID NO: 486 | HCDR2 | MIYYDSSKMYYADTVKG |
| (Kabat) | | |
| SEQ ID NO: 487 | HCDR3 | FWWDLDFDH |
| (Kabat) | | |
| SEQ ID NO: 489 | HCDR1 | GFTFSKQ |
| (Chothia) | | |
| SEQ ID NO: 490 | HCDR2 | YYDSSK |
| (Chothia) | | |
| SEQ ID NO: 487 | HCDR3 | FWWDLDFDH |
| (Chothia) | | |
| SEQ ID NO: 491 | HCDR1 | GFTFSKQG |
| (IMGT) | | |
| SEQ ID NO: 492 | HCDR2 | IYYDSSKM |
| (IMGT) | | |
| SEQ ID NO: 493 | HCDR3 | ASFWWDLDFDH |
| (IMGT) | | |
| SEQ ID NO: 494 | VH | EVKLVESGGDLVQPGDSLTLSCVASGFTFSKQ |
| | | GMHWIRQAPKKGLEWIAMIYYDSSKMYYAD |
| | | TVKGRFTISRDNSKNTLYLEMNSLRSEDTAM |
| | | YYCASFWWDLDFDHWGQGVMVTVSS |
| ANTI-TCR | | |
| SEQ ID NO: 495 | LCDR1 | SATSSVSYMH |
| (Combined) | | |
| SEQ ID NO: 421 | LCDR2 | DTSKLAS |
| (Combined) | | |
| SEQ ID NO: 496 | LCDR3 | QQWSSNPLT |
| (Combined) | | |
| SEQ ID NO: 495 | LCDR1 | SATSSVSYMH |
| (Kabat) | | |
| SEQ ID NO: 421 | LCDR2 | DTSKLAS |
| (Kabat) | | |
| SEQ ID NO: 496 | LCDR3 | QQWSSNPLT |
| (Kabat) | | |
| SEQ ID NO: 497 | LCDR1 | TSSVSY |
| (Chothia) | | |
| SEQ ID NO: 424 | LCDR2 | DTS |
| (Chothia) | | |
| SEQ ID NO: 498 | LCDR3 | WSSNPL |
| (Chothia) | | |
| SEQ ID NO: 426 | LCDR1 | SSVSY |
| (IMGT) | | |
| SEQ ID NO: 424 | LCDR2 | DTS |
| (IMGT) | | |
| SEQ ID NO: 496 | LCDR3 | QQWSSNPLT |
| (IMGT) | | |
| SEQ ID NO: 499 | VL | QIVLTQSPAIMSASPGEKVTMTCSATSSVSYM |
| | | HWYQQKSGTSPKRWIYDTSKLASGVPARFSG |
| | | SGSGTSYSLTISSMEAEDAATYYCQQWSSNPL |
| | | TFGAGTKLELK |
| SEQ ID NO: 500 | HCDR1 | GYKFTSYVMH |
| (Combined) | | |
| SEQ ID NO: 501 | HCDR2 | YINPYNDVTKYNEKFKG |
| (Combined) | | |
| SEQ ID NO: 502 | HCDR3 | GSYYDYDGFVY |
| (Combined) | | |
| SEQ ID NO: 503 | HCDR1 | SYVMH |
| (Kabat) | | |
| SEQ ID NO: 501 | HCDR2 | YINPYNDVTKYNEKFKG |
| (Kabat) | | |
| SEQ ID NO: 502 | HCDR3 | GSYYDYDGFVY |
| (Kabat) | | |
| SEQ ID NO: 504 | HCDR1 | GYKFTSY |
| (Chothia) | | |
| SEQ ID NO: 505 | HCDR2 | NPYNDV |
| (Chothia) | | |
| SEQ ID NO: 502 | HCDR3 | GSYYDYDGFVY |
| (Chothia) | | |
| SEQ ID NO: 506 | HCDR1 | GYKFTSYV |
| (IMGT) | | |
| SEQ ID NO: 507 | HCDR2 | INPYNDVT |
| (IMGT) | | |
| SEQ ID NO: 508 | HCDR3 | ARGSYYDYDGFVY |
| (IMGT) | | |
| SEQ ID NO: 509 | VH | EVQLQQSGPELVKPGASVKMSCKASGYKFTS |
| | | YVMHWVKQKPGQGLEWIGYINPYNDVTKYN |
| | | EKFKGKATLTSDKSSSTAYMELSSLTSEDSAV |
| | | HYCARGSYYDYDGFVYWGQGTLVTVSA |
| ANTI-CD28 (1) | | |
| SEQ ID NO: 510 | LCDR1 | HASQNIYVWLN |
| (Combined) | | |
| SEQ ID NO: 511 | LCDR2 | KASNLHT |
| (Combined) | | |
| SEQ ID NO: 512 | LCDR3 | QQGQTYPYT |
| (Combined) | | |
| SEQ ID NO: 510 | LCDR1 | HASQNIYVWLN |
| (Kabat) | | |
| SEQ ID NO: 511 | LCDR2 | KASNLHT |
| (Kabat) | | |
| SEQ ID NO: 512 | LCDR3 | QQGQTYPYT |
| (Kabat) | | |
| SEQ ID NO: 513 | LCDR1 | SQNIYVW |
| (Chothia) | | |
| SEQ ID NO: 514 | LCDR2 | KAS |
| (Chothia) | | |
| SEQ ID NO: 515 | LCDR3 | GQTYPY |
| (Chothia) | | |
| SEQ ID NO: 516 | LCDR1 | QNIYVW |
| (IMGT) | | |
| SEQ ID NO: 514 | LCDR2 | KAS |
| (IMGT) | | |
| SEQ ID NO: 512 | LCDR3 | QQGQTYPYT |
| (IMGT) | | |
| SEQ ID NO: 517 | VL | DIQMTQSPSSLSASVGDRVTITCHASQNIYVW |
| | | LNWYQQKPGKAPKLLIYKASNLHTGVPSRFS |
| | | GSGSGTDFTLTISSLQPEDFATYYCQQGQTYP |
| | | YTFGGGTKVEIK |
| SEQ ID NO: 518 | LC | DIQMTQSPSSLSASVGDRVTITCHASQNIYVW |
| | | LNWYQQKPGKAPKLLIYKASNLHTGVPSRFS |
| | | GSGSGTDFTLTISSLQPEDFATYYCQQGQTYP |
| | | YTFGGGTKVEIKRTVAAPSVFIFPPSDEQLKSG |
| | | TASVVCLLNNFYPREAKVQWKVDNALQSGN |
| | | SQESVTEQDSKDSTYSLSSTLTLSKADYEKHK |
| | | VYACEVTHQGLSSPVTKSFNRGEC |
| SEQ ID NO: 519 | HCDR1 | GYTFTSYYIH |
| (Combined) | | |
| SEQ ID NO: 520 | HCDR2 | CIYPGNVNTNYNEKFKD |
| (Combined) | | |
| SEQ ID NO: 521 | HCDR3 | SHYGLDWNFDV |
| (Combined) | | |
| SEQ ID NO: 522 | HCDR1 | SYYIH |
| (Kabat) | | |
| SEQ ID NO: 520 | HCDR2 | CIYPGNVNTNYNEKFKD |
| (Kabat) | | |
| SEQ ID NO: 521 | HCDR3 | SHYGLDWNFDV |
| (Kabat) | | |
| SEQ ID NO: 523 | HCDR1 | GYTFTSY |
| (Chothia) | | |
| SEQ ID NO: 524 | HCDR2 | YPGNVN |
| (Chothia) | | |
| SEQ ID NO: 521 | HCDR3 | SHYGLDWNFDV |
| (Chothia) | | |
| SEQ ID NO: 525 | HCDR1 | GYTFTSYY |
| (IMGT) | | |
| SEQ ID NO: 526 | HCDR2 | IYPGNVNT |
| (IMGT) | | |
| SEQ ID NO: 527 | HCDR3 | TRSHYGLDWNFDV |
| (IMGT) | | |
| SEQ ID NO: 528 | VH | QVQLVQSGAEVKKPGASVKVSCKASGYTFTS |
| | | YYIHWVRQAPGQGLEWIGCIYPGNVNTNYNE |
| | | KFKDRATLTVDTSISTAYMELSRLRSDDTAV |
| | | YFCTRSHYGLDWNFDVWGQGTTVTVSS |
| SEQ ID NO: 529 | HC | QVQLVQSGAEVKKPGASVKVSCKASGYTFTS |
| | | YYIHWVRQAPGQGLEWIGCIYPGNVNTNYNE |
| | | KFKDRATLTVDTSISTAYMELSRLRSDDTAV |
| | | YFCTRSHYGLDWNFDVWGQGTTVTVSSAST |
| | | KGPSVFPLAPSSKSTSGGTAALGCLVKDYFPE |
| | | PVTVSWNSGALTSGVHTFPAVLQSSGLYSLSS |
| | | VVTVPSSSLGTQTYICNVNHKPSNTKVDKRV |
| | | EPKSC |
| ANTI-CD28 (2) | | |
| SEQ ID NO: 530 | LCDR1 | SGSSSNIVSNYVN |
| (Combined) | | |
| SEQ ID NO: 531 | LCDR2 | DNNKRPS |
| (Combined) | | |
| SEQ ID NO: 532 | LCDR3 | QSYAIGSYSVV |
| (Combined) | | |
| SEQ ID NO: 530 | LCDR1 | SGSSSNIVSNYVN |
| (Kabat) | | |
| SEQ ID NO: 531 | LCDR2 | DNNKRPS |
| (Kabat) | | |
| SEQ ID NO: 532 | LCDR3 | QSYAIGSYSVV |
| (Kabat) | | |
| SEQ ID NO: 533 | LCDR1 | SSSNIVSNY |
| (Chothia) | | |
| SEQ ID NO: 534 | LCDR2 | DNN |
| (Chothia) | | |
| SEQ ID NO: 535 | LCDR3 | YAIGSYSV |
| (Chothia) | | |
| SEQ ID NO: 536 | LCDR1 | SSNIVSNY |
| (IMGT) | | |
| SEQ ID NO: 534 | LCDR2 | DNN |
| (IMGT) | | |
| SEQ ID NO: 532 | LCDR3 | QSYAIGSYSVV |
| (IMGT) | | |
| SEQ ID NO: 537 | VL | DIVLTQPPSVSGAPGQRVTISCSGSSSNIVSNY |
| | | VNWYQQLPGTAPKLLIYDNNKRPSGVPDRFS |
| | | GSKSGTSASLAITGLQSEDEADYYCQSYAIGS |
| | | YSVVFGGGTKLTVL |
| SEQ ID NO: 538 | HCDR1 | GFTFSTYGMS |
| (Combined) | | |
| SEQ ID NO: 539 | HCDR2 | SIFYTGSSTYYADSVKG |
| (Combined) | | |
| SEQ ID NO: 540 | HCDR3 | IGYAGDSKYAI |
| (Combined) | | |
| SEQ ID NO: 541 | HCDR1 | TYGMS |
| (Kabat) | | |
| SEQ ID NO: 539 | HCDR2 | SIFYTGSSTYYADSVKG |
| (Kabat) | | |
| SEQ ID NO: 540 | HCDR3 | IGYAGDSKYAI |
| (Kabat) | | |
| SEQ ID NO: 542 | HCDR1 | GFTFSTY |
| (Chothia) | | |
| SEQ ID NO: 543 | HCDR2 | FYTGSS |
| (Chothia) | | |
| SEQ ID NO: 540 | HCDR3 | IGYAGDSKYAI |
| (Chothia) | | |
| SEQ ID NO: 544 | HCDR1 | GFTFSTYG |
| (IMGT) | | |
| SEQ ID NO: 545 | HCDR2 | IFYTGSST |
| (IMGT) | | |
| SEQ ID NO: 546 | HCDR3 | ARIGYAGDSKYAI |
| (IMGT) | | |
| SEQ ID NO: 547 | VH | QVQLVESGGGLVQPGGSLRLSCAASGFTFST |
| | | YGMSWVRQAPGKGLEWVSSIFYTGSSTYYA |
| | | DSVKGRFTISRDNSKNTLYLQMNSLRAEDTA |
| | | VYYCARIGYAGDSKYAIWGQGTLVTVSS |
| SEQ ID NO: 548 | VH | EVQLVESGGGLVQPGGSLRLSCAASGFTFSTY |
| | | GMSWVRQAPGKGLEWVSSIFYTGSSTYYADS |
| | | VKGRFTISRDNSKNTLYLQMNSLRAEDTAVY |
| | | YCARIGYAGDSKYAIWGQGTLVTVSS |
| ANTI-ICOS | | |
| SEQ ID NO: 549 | LCDR1 | KSSQSLLSGSFNYLT |
| (Combined) | | |
| SEQ ID NO: 550 | LCDR2 | YASTRHT |
| (Combined) | | |
| SEQ ID NO: 551 | LCDR3 | HHHYNAPPT |
| (Combined) | | |
| SEQ ID NO: 549 | LCDR1 | KSSQSLLSGSFNYLT |
| (Kabat) | | |
| SEQ ID NO: 550 | LCDR2 | YASTRHT |
| (Kabat) | | |
| SEQ ID NO: 551 | LCDR3 | HHHYNAPPT |
| (Kabat) | | |
| SEQ ID NO: 552 | LCDR1 | SQSLLSGSFNY |
| (Chothia) | | |
| SEQ ID NO: 553 | LCDR2 | YAS |
| (Chothia) | | |
| SEQ ID NO: 554 | LCDR3 | HYNAPP |
| (Chothia) | | |
| SEQ ID NO: 555 | LCDR1 | QSLLSGSFNY |
| (IMGT) | | |
| SEQ ID NO: 553 | LCDR2 | YAS |
| (IMGT) | | |
| SEQ ID NO: 551 | LCDR3 | HHHYNAPPT |
| (IMGT) | | |
| SEQ ID NO: 556 | VL | DIVMTQSPDSLAVSLGERATINCKSSQSLLSGS |
| | | FNYLTWYQQKPGQPPKLLIFYASTRHTGVPD |
| | | RFSGSGSGTDFTLTISSLQAEDVAVYYCHHHY |
| | | NAPPTFGPGTKVDIK |
| SEQ ID NO: 557 | LC | DIVMTQSPDSLAVSLGERATINCKSSQSLLSGS |
| | | FNYLTWYQQKPGQPPKLLIFYASTRHTGVPD |
| | | RFSGSGSGTDFTLTISSLQAEDVAVYYCHHHY |
| | | NAPPTFGPGTKVDIKRTVAAPSVFIFPPSDEQL |
| | | KSGTASVVCLLNNFYPREAKVQWKVDNALQ |
| | | SGNSQESVTEQDSKDSTYSLSSTLTLSKADYE |
| | | KHKVYACEVTHQGLSSPVTKSFNRGEC |
| SEQ ID NO: 558 | HCDR1 | GFTFSDYWMD |
| (Combined) | | |
| SEQ ID NO: 559 | HCDR2 | NIDEDGSITEYSPFVKG |
| (Combined) | | |
| SEQ ID NO: 560 | HCDR3 | WGRFGFDS |
| (Combined) | | |
| SEQ ID NO: 561 | HCDR1 | DYWMD |
| (Kabat) | | |
| SEQ ID NO: 559 | HCDR2 | NIDEDGSITEYSPFVKG |
| (Kabat) | | |
| SEQ ID NO: 560 | HCDR3 | WGRFGFDS |
| (Kabat) | | |
| SEQ ID NO: 562 | HCDR1 | GFTFSDY |
| (Chothia) | | |
| SEQ ID NO: 563 | HCDR2 | DEDGSI |
| (Chothia) | | |
| SEQ ID NO: 560 | HCDR3 | WGRFGFDS |
| (Chothia) | | |
| SEQ ID NO: 564 | HCDR1 | GFTFSDYW |
| (IMGT) | | |
| SEQ ID NO: 565 | HCDR2 | IDEDGSIT |
| (IMGT) | | |
| SEQ ID NO: 566 | HCDR3 | TRWGRFGFDS |
| (IMGT) | | |
| SEQ ID NO: 567 | VH | EVQLVESGGGLVQPGGSLRLSCAASGFTFSD |
| | | YWMDWVRQAPGKGLVWVSNIDEDGSITEYS |
| | | PFVKGRFTISRDNAKNTLYLQMNSLRAEDTA |
| | | VYYCTRWGRFGFDSWGQGTLVTVSS |
| ANTI-CD27 | | |
| SEQ ID NO: 568 | LCDR1 | RASQGISRWLA |
| (Combined) | | |
| SEQ ID NO: 55 | LCDR2 | AASSLQS |
| (Combined) | | |
| SEQ ID NO: 569 | LCDR3 | QQYNTYPRT |
| (Combined) | | |
| SEQ ID NO: 568 | LCDR1 | RASQGISRWLA |
| (Kabat) | | |
| SEQ ID NO: 55 | LCDR2 | AASSLQS |
| (Kabat) | | |
| SEQ ID NO: 569 | LCDR3 | QQYNTYPRT |
| (Kabat) | | |
| SEQ ID NO: 570 | LCDR1 | SQGISRW |
| (Chothia) | | |
| SEQ ID NO: 58 | LCDR2 | AAS |
| (Chothia) | | |
| SEQ ID NO: 571 | LCDR3 | YNTYPR |
| (Chothia) | | |
| SEQ ID NO: 572 | LCDR1 | QGISRW |
| (IMGT) | | |
| SEQ ID NO: 58 | LCDR2 | AAS |
| (IMGT) | | |
| SEQ ID NO: 569 | LCDR3 | QQYNTYPRT |
| (IMGT) | | |
| SEQ ID NO: 573 | VL | DIQMTQSPSSLSASVGDRVTITCRASQGISRW |
| | | LAWYQQKPEKAPKSLIYAASSLQSGVPSRFSG |
| | | SGSGTDFTLTISSLQPEDFATYYCQQYNTYPR |
| | | TFGQGTKVEIK |
| SEQ ID NO: 574 | HCDR1 | GFTFSSYDMH |
| (Combined) | | |
| SEQ ID NO: 575 | HCDR2 | VIWYDGSNKYYADSVKG |
| (Combined) | | |
| SEQ ID NO: 576 | HCDR3 | GSGNWGFFDY |
| (Combined) | | |
| SEQ ID NO: 577 | HCDR1 | SYDMH |
| (Kabat) | | |
| SEQ ID NO: 575 | HCDR2 | VIWYDGSNKYYADSVKG |
| (Kabat) | | |
| SEQ ID NO: 576 | HCDR3 | GSGNWGFFDY |
| (Kabat) | | |
| SEQ ID NO: 47 | HCDR1 | GFTFSSY |
| (Chothia) | | |
| SEQ ID NO: 578 | HCDR2 | WYDGSN |
| (Chothia) | | |
| SEQ ID NO: 576 | HCDR3 | GSGNWGFFDY |
| (Chothia) | | |
| SEQ ID NO: 579 | HCDR1 | GFTFSSYD |
| (IMGT) | | |
| SEQ ID NO: 580 | HCDR2 | IWYDGSNK |
| (IMGT) | | |
| SEQ ID NO: 581 | HCDR3 | ARGSGNWGFFDY |
| (IMGT) | | |
| SEQ ID NO: 582 | VH | QVQLVESGGGVVQPGRSLRLSCAASGFTFSS |
| | | YDMHWVRQAPGGLEWVAVIWYDGSNKYYA |
| | | DSVKGRFTISRDNSKNTLYLQMNSLRAEDTA |
| | | VYYCARGSGNWGFFDYWGQGTLVTVSS |
| ANTI-CD25 (1) | | |
| SEQ ID NO: 583 | LCDR1 | SASSSRSYMQ |
| (Combined) | | |
| SEQ ID NO: 421 | LCDR2 | DTSKLAS |
| (Combined) | | |
| SEQ ID NO: 584 | LCDR3 | HQRSSYT |
| (Combined) | | |
| SEQ ID NO: 583 | LCDR1 | SASSSRSYMQ |
| (Kabat) | | |
| SEQ ID NO: 421 | LCDR2 | DTSKLAS |
| (Kabat) | | |
| SEQ ID NO: 584 | LCDR3 | HQRSSYT |
| (Kabat) | | |
| SEQ ID NO: 585 | LCDR1 | SSSRSY |
| (Chothia) | | |
| SEQ ID NO: 424 | LCDR2 | DTS |
| (Chothia) | | |
| SEQ ID NO: 586 | LCDR3 | RSSY |
| (Chothia) | | |
| SEQ ID NO: 587 | LCDR1 | SSRSY |
| (IMGT) | | |
| SEQ ID NO: 424 | LCDR2 | DTS |
| (IMGT) | | |
| SEQ ID NO: 584 | LCDR3 | HQRSSYT |
| (IMGT) | | |
| SEQ ID NO: 588 | VL | QIVSTQSPAIMSASPGEKVTMTCSASSSRSYM |
| | | QWYQQKPGTSPKRWIYDTSKLASGVPARFSG |
| | | SGSGTSYSLTISSMEAEDAATYYCHQRSSYTF |
| | | GGGTKLEIK |
| SEQ ID NO: 589 | HCDR1 | GYSFTRYWMH |
| (Combined) | | |
| SEQ ID NO: 590 | HCDR2 | AIYPGNSDTSYNQKFEG |
| (Combined) | | |
| SEQ ID NO: 591 | HCDR3 | DYGYYFDF |
| (Combined) | | |
| SEQ ID NO: 592 | HCDR1 | RYWMH |
| (Kabat) | | |
| SEQ ID NO: 590 | HCDR2 | AIYPGNSDTSYNQKFEG |
| (Kabat) | | |
| SEQ ID NO: 591 | HCDR3 | DYGYYFDF |
| (Kabat) | | |
| SEQ ID NO: 593 | HCDR1 | GYSFTRY |
| (Chothia) | | |
| SEQ ID NO: 594 | HCDR2 | YPGNSD |
| (Chothia) | | |
| SEQ ID NO: 591 | HCDR3 | DYGYYFDF |
| (Chothia) | | |
| SEQ ID NO: 595 | HCDR1 | GYSFTRYW |
| (IMGT) | | |
| SEQ ID NO: 596 | HCDR2 | IYPGNSDT |
| (IMGT) | | |
| SEQ ID NO: 597 | HCDR3 | SRDYGYYFDF |
| (IMGT) | | |
| SEQ ID NO: 598 | VH | EVQLQQSGTVLARPGASVKMSCKASGYSFTR |
| | | YWMHWIKQRPGQGLEWIGAIYPGNSDTSYN |
| | | QKFEGKAKLTAVTSASTAYMELSSLTHEDSA |
| | | VYYCSRDYGYYFDFWGQGTTLTVSS |
| ANTI-CD25 (2) | | |
| SEQ ID NO: 599 | LCDR1 | KASQSVDYDGDSYMN |
| (Combined) | | |
| SEQ ID NO: 600 | LCDR2 | AASNLES |
| (Combined) | | |
| SEQ ID NO: 601 | LCDR3 | QQSNEDPYT |
| (Combined) | | |
| SEQ ID NO: 599 | LCDR1 | KASQSVDYDGDSYMN |
| (Kabat) | | |
| SEQ ID NO: 600 | LCDR2 | AASNLES |
| (Kabat) | | |
| SEQ ID NO: 601 | LCDR3 | QQSNEDPYT |
| (Kabat) | | |
| SEQ ID NO: 602 | LCDR1 | SQSVDYDGDSY |
| (Chothia) | | |
| SEQ ID NO: 58 | LCDR2 | AAS |
| (Chothia) | | |
| SEQ ID NO: 603 | LCDR3 | SNEDPY |
| (Chothia) | | |
| SEQ ID NO: 604 | LCDR1 | QSVDYDGDSY |
| (IMGT) | | |
| SEQ ID NO: 58 | LCDR2 | AAS |
| (IMGT) | | |
| SEQ ID NO: 601 | LCDR3 | QQSNEDPYT |
| (IMGT) | | |
| SEQ ID NO: 605 | VL | DIVLTQSPLSLPVTLGQPASISCKASQSVDYDG |
| | | DSYMNWYQQRPGQSPRLLIYAASNLESGVPD |
| | | RFSGSGSGTDFTLKISRVEAEDVGVYYCQQSN |
| | | EDPYTFGGGTKVEIK |
| SEQ ID NO: 606 | LC | DIVLTQSPLSLPVTLGQPASISCKASQSVDYDG |
| | | DSYMNWYQQRPGQSPRLLIYAASNLESGVPD |
| | | RFSGSGSGTDFTLKISRVEAEDVGVYYCQQSN |
| | | EDPYTFGGGTKVEIKRTVAAPSVFIFPPSDEQL |
| | | KSGTASVVCLLNNFYPREAKVQWKVDNALQ |
| | | SGNSQESVTEQDSKDSTYSLSSTLTLSKADYE |
| | | KHKVYACEVTHQGLSSPVTKSFNRGEC |
| SEQ ID NO: 607 | HCDR1 | GYAFTNYLIE |
| (Combined) | | |
| SEQ ID NO: 608 | HCDR2 | VINPGSGGTNYNEKFKG |
| (Combined) | | |
| SEQ ID NO: 609 | HCDR3 | WRGDGYYAYFDV |
| (Combined) | | |
| SEQ ID NO: 610 | HCDR1 | NYLIE |
| (Kabat) | | |
| SEQ ID NO: 608 | HCDR2 | VINPGSGGTNYNEKFKG |
| (Kabat) | | |
| SEQ ID NO: 609 | HCDR3 | WRGDGYYAYFDV |
| (Kabat) | | |
| SEQ ID NO: 611 | HCDR1 | GYAFTNY |
| (Chothia) | | |
| SEQ ID NO: 612 | HCDR2 | NPGSGG |
| (Chothia) | | |
| SEQ ID NO: 609 | HCDR3 | WRGDGYYAYFDV |
| (Chothia) | | |
| SEQ ID NO: 613 | HCDR1 | GYAFTNYL |
| (IMGT) | | |
| SEQ ID NO: 614 | HCDR2 | INPGSGGT |
| (IMGT) | | |
| SEQ ID NO: 615 | HCDR3 | ARWRGDGYYAYFDV |
| (IMGT) | | |
| SEQ ID NO: 616 | VH | EVQLVQSGAEVKKPGESLKISCKGSGYAFTN |
| | | YLIEWVRQMPGKGLEWMGVINPGSGGTNYN |
| | | EKFKGQVTISADKSISTAYLQWSSLKASDTA |
| | | MYYCARWRGDGYYAYFDVWGQGTTVTVSS |
| SEQ ID NO: 617 | HC | EVQLVQSGAEVKKPGESLKISCKGSGYAFTN |
| | | YLIEWVRQMPGKGLEWMGVINPGSGGTNYN |
| | | EKFKGQVTISADKSISTAYLQWSSLKASDTA |
| | | MYYCARWRGDGYYAYFDVWGQGTTVTVSS |
| | | ASTKGPSVFPLAPSSKSTSGGTAALGCLVKDY |
| | | FPEPVTVSWNSGALTSGVHTFPAVLQSSGLYS |
| | | LSSVVTVPSSSLGTQTYICNVNHKPSNTKVDK |
| | | RVEPKSC |
| ANTI-4-1BB | | |
| SEQ ID NO: 618 | LCDR1 | RASQSVSSYLA |
| (Combined) | | |
| SEQ ID NO: 619 | LCDR2 | DASNRAT |
| (Combined) | | |
| SEQ ID NO: 620 | LCDR3 | QQRSNWPPALT |
| (Combined) | | |
| SEQ ID NO: 618 | LCDR1 | RASQSVSSYLA |
| (Kabat) | | |
| SEQ ID NO: 619 | LCDR2 | DASNRAT |
| (Kabat) | | |
| SEQ ID NO: 620 | LCDR3 | QQRSNWPPALT |
| (Kabat) | | |
| SEQ ID NO: 621 | LCDR1 | SQSVSSY |
| (Chothia) | | |
| SEQ ID NO: 622 | LCDR2 | DAS |
| (Chothia) | | |
| SEQ ID NO: 623 | LCDR3 | RSNWPPAL |
| (Chothia) | | |
| SEQ ID NO: 624 | LCDR1 | QSVSSY |
| (IMGT) | | |
| SEQ ID NO: 622 | LCDR2 | DAS |
| (IMGT) | | |
| SEQ ID NO: 620 | LCDR3 | QQRSNWPPALT |
| (IMGT) | | |
| SEQ ID NO: 625 | VL | EIVLTQSPATLSLSPGERATLSCRASQSVSSYL |
| | | AWYQQKPGQAPRLLIYDASNRATGIPARFSG |
| | | SGSGTDFTLTISSLEPEDFAVYYCQQRSNWPP |
| | | ALTFCGGTKVEIK |
| SEQ ID NO: 626 | HCDR1 | GGSFSGYYWS |
| (Combined) | | |
| SEQ ID NO: 627 | HCDR2 | EINHGGYVTYNPSLES |
| (Combined) | | |
| SEQ ID NO: 628 | HCDR3 | DYGPGNYDWYFDL |
| (Combined) | | |
| SEQ ID NO: 629 | HCDR1 | GYYWS |
| (Kabat) | | |
| SEQ ID NO: 627 | HCDR2 | EINHGGYVTYNPSLES |
| (Kabat) | | |
| SEQ ID NO: 628 | HCDR3 | DYGPGNYDWYFDL |
| (Kabat) | | |
| SEQ ID NO: 630 | HCDR1 | GGSFSGY |
| (Chothia) | | |
| SEQ ID NO: 631 | HCDR2 | NHGGY |
| (Chothia) | | |
| SEQ ID NO: 628 | HCDR3 | DYGPGNYDWYFDL |
| (Chothia) | | |
| SEQ ID NO: 632 | HCDR1 | GGSFSGYY |
| (IMGT) | | |
| SEQ ID NO: 633 | HCDR2 | INHGGYV |
| (IMGT) | | |
| SEQ ID NO: 634 | HCDR3 | ARDYGPGNYDWYFDL |
| (IMGT) | | |
| SEQ ID NO: 635 | VH | QVQLQQWGAGLLKPSETLSLTCAVYGGSFSG |
| | | YYWSWIRQSPEKGLEWIGEINHGGYVTYNPS |
| | | LESRVTISVDTSKNQFSLKLSSVTAADTAVYY |
| | | CARDYGPGNYDWYFDLWGRGTLVTVSS |
| ANTI-IL6RA | | |
| SEQ ID NO: 636 | LCDR1 | RASQDISSYLN |
| (Combined) | | |
| SEQ ID NO: 637 | LCDR2 | YTSRLHS |
| (Combined) | | |
| SEQ ID NO: 300 | LCDR3 | QQGNTLPYT |
| (Combined) | | |
| SEQ ID NO: 636 | LCDR1 | RASQDISSYLN |
| (Kabat) | | |
| SEQ ID NO: 637 | LCDR2 | YTSRLHS |
| (Kabat) | | |
| SEQ ID NO: 300 | LCDR3 | QQGNTLPYT |
| (Kabat) | | |
| SEQ ID NO: 638 | LCDR1 | SQDISSY |
| (Chothia) | | |
| SEQ ID NO: 639 | LCDR2 | YTS |
| (Chothia) | | |
| SEQ ID NO: 640 | LCDR3 | GNTLPY |
| (Chothia) | | |
| SEQ ID NO: 641 | LCDR1 | QDISSY |
| (IMGT) | | |
| SEQ ID NO: 639 | LCDR2 | YTS |
| (IMGT) | | |
| SEQ ID NO: 300 | LCDR3 | QQGNTLPYT |
| (IMGT) | | |
| SEQ ID NO: 642 | VL | DIQMTQSPSSLSASVGDRVTITCRASQDISSYL |
| | | NWYQQKPGKAPKLLIYYTSRLHSGVPSRFSG |
| | | SGSGTDFTFTISSLQPEDIATYYCQQGNTLPYT |
| | | FGQGTKVEIK |
| SEQ ID NO: 643 | HCDR1 | GYSITSDHAWS |
| (Combined) | | |
| SEQ ID NO: 644 | HCDR2 | YISYSGITTYNPSLKS |
| (Combined) | | |
| SEQ ID NO: 645 | HCDR3 | SLARTTAMDY |
| (Combined) | | |
| SEQ ID NO: 646 | HCDR1 | SDHAWS |
| (Kabat) | | |
| SEQ ID NO: 644 | HCDR2 | YISYSGITTYNPSLKS |
| (Kabat) | | |
| SEQ ID NO: 645 | HCDR3 | SLARTTAMDY |
| (Kabat) | | |
| SEQ ID NO: 647 | HCDR1 | GYSITSDH |
| (Chothia) | | |
| SEQ ID NO: 648 | HCDR2 | SYSGI |
| (Chothia) | | |
| SEQ ID NO: 645 | HCDR3 | SLARTTAMDY |
| (Chothia) | | |
| SEQ ID NO: 649 | HCDR1 | GYSITSDHA |
| (IMGT) | | |
| SEQ ID NO: 650 | HCDR2 | ISYSGIT |
| (IMGT) | | |
| SEQ ID NO: 651 | HCDR3 | ARSLARTTAMDY |
| (IMGT) | | |
| SEQ ID NO: 652 | VH | QVQLQESGPGLVRPSQTLSLTCTVSGYSITSD |
| | | HAWSWVRQPPGRGLEWIGYISYSGITTYNPSL |
| | | KSRVTMLRDTSKNQFSLRLSSVTAADTAVYY |
| | | CARSLARTTAMDYWGQGSLVTVSS |
| ANTI-IL6RB | | |
| SEQ ID NO: 54 | LCDR1 | RASQSISSYLN |
| (Combined) | | |
| SEQ ID NO: 55 | LCDR2 | AASSLQS |
| (Combined) | | |
| SEQ ID NO: 653 | LCDR3 | QQSYSTPPIT |
| (Combined) | | |
| SEQ ID NO: 54 | LCDR1 | RASQSISSYLN |
| (Kabat) | | |
| SEQ ID NO: 55 | LCDR2 | AASSLQS |
| (Kabat) | | |
| SEQ ID NO: 653 | LCDR3 | QQSYSTPPIT |
| (Kabat) | | |
| SEQ ID NO: 57 | LCDR1 | SQSISSY |
| (Chothia) | | |
| SEQ ID NO: 58 | LCDR2 | AAS |
| (Chothia) | | |
| SEQ ID NO: 654 | LCDR3 | SYSTPPI |
| (Chothia) | | |
| SEQ ID NO: 60 | LCDR1 | QSISSY |
| (IMGT) | | |
| SEQ ID NO: 58 | LCDR2 | AAS |
| (IMGT) | | |
| SEQ ID NO: 653 | LCDR3 | QQSYSTPPIT |
| (IMGT) | | |
| SEQ ID NO: 655 | VL | DIQMTQSPSSLSASVGDRVTITCRASQSISSYL |
| | | NWYQQKPGKAPKLLIYAASSLQSGVPSRFSG |
| | | SGSGTDFTLTISSLQPEDFATYYCQQSYSTPPI |
| | | TFGQGTRLEIK |
| SEQ ID NO: 656 | HCDR1 | GFTFSDYYMT |
| (Combined) | | |
| SEQ ID NO: 657 | HCDR2 | YISSSGTNKYNADSVKG |
| (Combined) | | |
| SEQ ID NO: 658 | HCDR3 | DPPWGMDV |
| (Combined) | | |
| SEQ ID NO: 659 | HCDR1 | DYYMT |
| (Kabat) | | |
| SEQ ID NO: 657 | HCDR2 | YISSSGTNKYNADSVKG |
| (Kabat) | | |
| SEQ ID NO: 658 | HCDR3 | DPPWGMDV |
| (Kabat) | | |
| SEQ ID NO: 562 | HCDR1 | GFTFSDY |
| (Chothia) | | |
| SEQ ID NO: 660 | HCDR2 | SSSGTN |
| (Chothia) | | |
| SEQ ID NO: 658 | HCDR3 | DPPWGMDV |
| (Chothia) | | |
| SEQ ID NO: 661 | HCDR1 | GFTFSDYY |
| (IMGT) | | |
| SEQ ID NO: 662 | HCDR2 | ISSSGTNK |
| (IMGT) | | |
| SEQ ID NO: 663 | HCDR3 | VRDPPWGMDV |
| (IMGT) | | |
| SEQ ID NO: 664 | VH | QVQLVESGGGLVKPGGSLRLSCAASGFTFSD |
| | | YYMTWIRQTPGKGLDWVSYISSSGTNKYNA |
| | | DSVKGRFTISRDNAKNSLYLQMNSLRAEDTA |
| | | VYYCVRDPPWGMDVWGQGTTVTVSS |
| ANTI-CD2 (1) | | |
| SEQ ID NO: 665 | LCDR1 | RSSQSLLHSSGNTYLN |
| (Combined) | | |
| SEQ ID NO: 666 | LCDR2 | LVSKLES |
| (Combined) | | |
| SEQ ID NO: 667 | LCDR3 | MQFTHYPYT |
| (Combined) | | |
| SEQ ID NO: 665 | LCDR1 | RSSQSLLHSSGNTYLN |
| (Kabat) | | |
| SEQ ID NO: 666 | LCDR2 | LVSKLES |
| (Kabat) | | |
| SEQ ID NO: 667 | LCDR3 | MQFTHYPYT |
| (Kabat) | | |
| SEQ ID NO: 668 | LCDR1 | SQSLLHSSGNTY |
| (Chothia) | | |
| SEQ ID NO: 669 | LCDR2 | LVS |
| (Chothia) | | |
| SEQ ID NO: 670 | LCDR3 | FTHYPY |
| (Chothia) | | |
| SEQ ID NO: 671 | LCDR1 | QSLLHSSGNTY |
| (IMGT) | | |
| SEQ ID NO: 669 | LCDR2 | LVS |
| (IMGT) | | |
| SEQ ID NO: 667 | LCDR3 | MQFTHYPYT |
| (IMGT) | | |
| SEQ ID NO: 672 | VL | DVVMTQSPPSLLVTLGQPASISCRSSQSLLHSS |
| | | GNTYLNWLLQRPGQSPQPLIYLVSKLESGVPD |
| | | RFSGSGSGTDFTLKISGVEAEDVGVYYCMQF |
| | | THYPYTFGQGTKLEIK |
| SEQ ID NO: 673 | LC | DVVMTQSPPSLLVTLGQPASISCRSSQSLLHSS |
| | | GNTYLNWLLQRPGQSPQPLIYLVSKLESGVPD |
| | | RFSGSGSGTDFTLKISGVEAEDVGVYYCMQF |
| | | THYPYTFGQGTKLEIKRTVAAPSVFIFPPSDEQ |
| | | LKSGTASVVCLLNNFYPREAKVQWKVDNAL |
| | | QSGNSQESVTEQDSKDSTYSLSSTLTLSKADY |
| | | EKHKVYACEVTHQGLSSPVTKSFNRGEC |
| SEQ ID NO: 674 | HCDR1 | GYIFTEYYMY |
| (Combined) | | |
| SEQ ID NO: 675 | HCDR2 | RIDPEDGSIDYVEKFKK |
| (Combined) | | |
| SEQ ID NO: 676 | HCDR3 | GKFNYRFAY |
| (Combined) | | |
| SEQ ID NO: 677 | HCDR1 | EYYMY |
| (Kabat) | | |
| SEQ ID NO: 675 | HCDR2 | RIDPEDGSIDYVEKFKK |
| (Kabat) | | |
| SEQ ID NO: 676 | HCDR3 | GKFNYRFAY |
| (Kabat) | | |
| SEQ ID NO: 678 | HCDR1 | GYIFTEY |
| (Chothia) | | |
| SEQ ID NO: 679 | HCDR2 | DPEDGS |
| (Chothia) | | |
| SEQ ID NO: 676 | HCDR3 | GKFNYRFAY |
| (Chothia) | | |
| SEQ ID NO: 680 | HCDR1 | GYIFTEYY |
| (IMGT) | | |
| SEQ ID NO: 681 | HCDR2 | IDPEDGSI |
| (IMGT) | | |
| SEQ ID NO: 682 | HCDR3 | ARGKFNYRFAY |
| (IMGT) | | |
| SEQ ID NO: 683 | VH | QVQLVQSGAEVQRPGASVKVSCKASGYIFTE |
| | | YYMYWVRQAPGQGLELVGRIDPEDGSIDYVE |
| | | KFKKKVTLTADTSSSTAYMELSSLTSDDTAV |
| | | YYCARGKFNYRFAYWGQGTLVTVSS |
| SEQ ID NO: 684 | HC | QVQLVQSGAEVQRPGASVKVSCKASGYIFTE |
| | | YYMYWVRQAPGQGLELVGRIDPEDGSIDYVE |
| | | KFKKKVTLTADTSSSTAYMELSSLTSDDTAV |
| | | YYCARGKFNYRFAYWGQGTLVTVSSASTKG |
| | | PSVFPLAPSSKSTSGGTAALGCLVKDYFPEPV |
| | | TVSWNSGALTSGVHTFPAVLQSSGLYSLSSV |
| | | VTVPSSSLGTQTYICNVNHKPSNTKVDKKVEP |
| | | KSC |

In some embodiments, the agent that stimulates a CD3/TCR complex and the agent that stimulates a costimulatory molecule and/or growth factor receptor are comprised in a multispecific binding molecule. Thus, contemplated herein are multispecific binding molecules comprising an agent that stimulates a CD3/TCR complex and an agent that stimulates a costimulatory molecule and/or growth factor receptor, such as but not limited to a multispecific binding molecule comprising a CD3 antigen binding domain and one or more of a CD28, ICOS, CD27, CD25, 4-1BB, IL6RA, IL6RB, and/or CD2 antigen binding domain. Non-limiting examples of such binding domains, as noted above, are provided above, for example in Table 19 and the publications incorporated by reference herein.

In some embodiments, the multispecific binding molecule comprises a CD3 antigen binding domain and a CD28 or CD2 antigen binding domain. In some embodiments, the CD3 antigen binding domain is an anti-CD3 antibody, optionally the anti-CD3 (1), anti-CD3 (2), anti-CD3 (3), or anti-CD3 (4) provided in Table 19, or an antibody fragment comprising one or more CDRs, VH, and/or VL thereof In some embodiments, the CD28 antigen binding domain is an anti-CD28 antibody, optionally the anti-CD28 (1) or anti-CD28 (2) provided in Table 19, or an antibody fragment comprising one or more CDRs, VH heavy chain, VL, and/or light chain thereof In some embodiments, the CD2 antigen binding domain is an anti-CD2 antibody, optionally the anti-CD2 (1) provided in Table 19, or an antibody fragment comprising one or more CDRs, VH, heavy chain, VL, and/or light chain thereof.

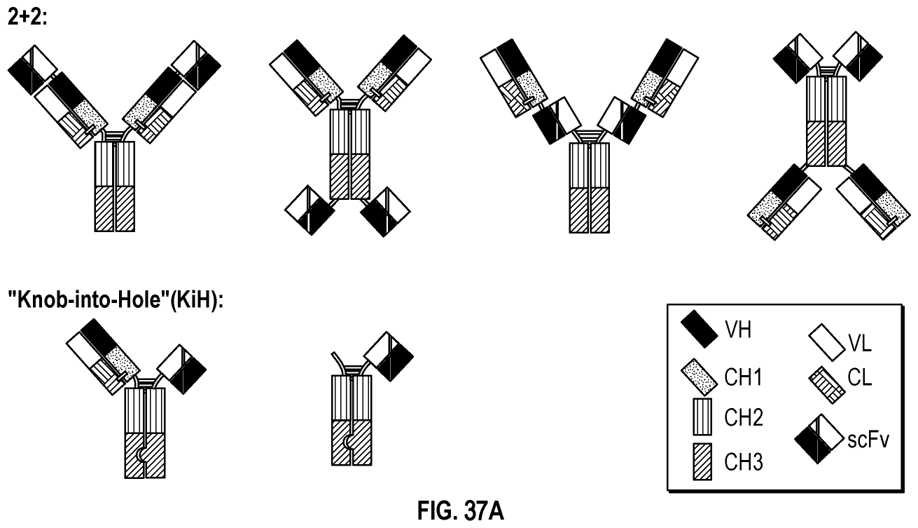

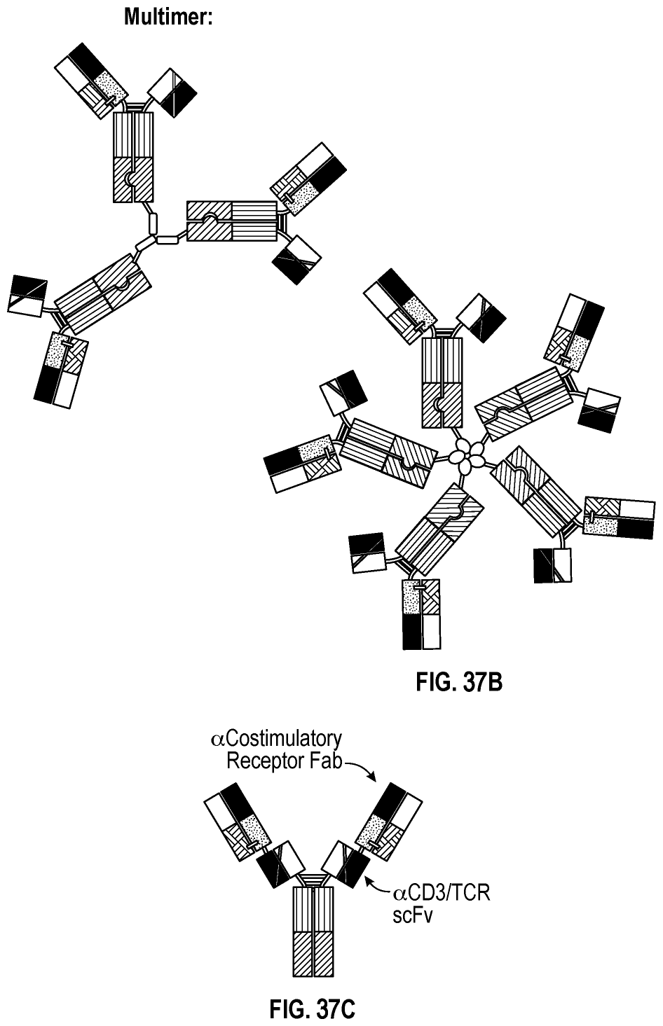

In some embodiments, the multispecific binding molecules comprise one or more heavy and/or light chains. Non-limiting exemplary heavy and light chain sequences that may be comprised in these multispecific binding molecules are provided in Table 20 below. Non-limiting exemplary combinations thereof are suggested in Table 20 based on the categorization of the recited heavy and/or light chains as within a Construct. This Construct organization provides examples of configurations of heavy and/or light chains but that further combinations and permutations thereof are also possible. Non-limiting examples of the format of any of these Constructs is provided in FIG. 37A-B, 48A-B, 49A-B, and 50A-B.

In some embodiments, the multispecific binding molecule comprises one or more heavy and/or light chain sequences disclosed in Table 20, or a sequence having at least 70%, 75%, 80%, 85%, 90%, 95%, or 99% identity thereto.

TABLE 20
Exemplary Fc, heavy chain (HC), and light chain (LC) sequences

| SEQ ID | | |
|---|---|---|
| NO: | Chain | Amino acid sequence |
| 701 | Wild type Fc | DKTHTCPPCPAPELLGGPSVFLFPPKPKDTLMISRTPEVT |
| | | CVVVDVSHEDPEVKFNWYVDGVEVHNAKTKPREEQYN |
| | | STYRVVSVLTVLHQDWLNGKEYKCKVSNKALPAPIEKTI |
| | | SKAKGQPREPQVYTLPPSREEMTKNQVSLTCLVKGFYPS |
| | | DIAVEWESNGQPENNYKTTPPVLDSDGSFFLYSKLTVDK |
| | | SRWQQGNVFSCSVMHEALHNHYTQKSLSLSPGK |
| 702 | Fc DANAPA | DKTHTCPPCPAPELLGGPSVFLFPPKPKDTLMISRTPEVT |
| | | CVVVAVSHEDPEVKFNWYVDGVEVHNAKTKPREEQYA |
| | | STYRVVSVLTVLHQDWLNGKEYKCKVSNKALAAPIEKT |
| | | ISKAKGQPREPQVYTLPPSREEMTKNQVSLTCLVKGFYP |
| | | SDIAVEWESNGQPENNYKTTPPVLDSDGSFFLYSKLTVD |
| | | KSRWQQGNVFSCSVMHEALHNHYTQKSLSLSPGK |
| 879 | Human IgG1 | apeaaggpsvflfppkpkdtlmisrtpevtcvvvdvshedpevkfnwyvdgvevhn |
| | Fc variant | aktkpreeqynstyrvvsvltvlhqdwlngkeykckvsnkalaapiektiskakgq |
| | L234A/L235A/ | prepqvytlppsrdeltknqvsltclvkgfypsdiavewesngqpennykttppvl |
| | P329A | dsdgsfflyskltvdksrwqqgnvfscsvmhealhnhytqkslslspgk |
| | (LALAPA) | |
| 880 | Human IgG1 | apeaagapsvflfppkpkdtlmisrtpevtcvvvdvshedpevkfnwyvdgvevhn |
| | Fc variant | aktkpreeqynstyrvvsvltvlhqdwlngkeykckvsnkalpapiektiskakgq |
| | L234A/L235A/ | prepqvytlppsrdeltknqvsltclvkgfypsdiavewesngqpennykttppvl |
| | G237A | dsdgsfflyskltvdksrwqqgnvfscsvmhealhnhytqkslslspgk |
| | (LALAGA) | |
| 881 | Human IgG1 | apeaaggpsvflfppkpkdtlmisrtpevtcvvvdvshedpevkfnwyvdgvevhn |
| | Fc variant | aktkpreeqynstyrvvsvltvlhqdwlngkeykckvsnkalgapiektiskakgq |
| | L234A/L235A/ | prepqvytlppsrdeltknqvsltclvkgfypsdiavewesngqpennykttppvl |
| | P329G | dsdgsfflyskltvdksrwqqgnvfscsvmhealhnhytqkslslspgk |
| | (LALAPG) | |
| 882 | Fc LALAPG | DKTHTCPPCPAPEAAGGPSVFLFPPKPKDTLMISRTPEVT |
| | (L234A/ | CVVVDVSHEDPEVKFNWYVDGVEVHNAKTKPREEQYN |
| | L235A/P329G) | STYRVVSVLTVLHQDWLNGKEYKCKVSNKALGAPIEKT |
| | | ISKAKGQPREPQVYTLPPSRREEMKNQVSLTCLVKGFYP |
| | | SDIAVEWESNGQPENNYKTTPPVLDSDGSFFLYSKLTVD |
| | | KSRWQQGNVFSCSVMHEALHNHYTQKSLSLSPGK |
| 883 | Human IgG1 | apellggpsvflfppkpkdtlmisrtpevtcvvvavshedpevkfnwyvdgvevhn |
| | Fc variant | aktkpreeqynstyrvvsvltvlhqdwlngkeykckvsnkalaapiektiskakgq |
| | D265A/P329A | prepqvytlppsrdeltknqvsltclvkgfypsdiavewesngqpennykttppvl |
| | (DAPA) | dsdgsfflyskltvdksrwqqgnvfscsvmhealhnhytqkslslspgk |
| 884 | Fc DAPA | DKTHTCPPCPAPELLGGPSVFLFPPKPKDTLMISRTPEVT |
| | (D265A/ | CVVVAVSHEDPEVKFNWYVDGVEVHNAKTKPREEQYN |
| | P329A) | STYRVVSVLTVLHQDWLNGKEYKCKVSNKALAAPIEKT |
| | | ISKAKGQPREPQVYTLPPSREEMTKNQVSLTCLVKGFYP |
| | | SDIAVEWESNGQPENNYKTTPPVLDSDGSFFLYSKLTVD |
| | | KSRWQQGNVFSCSVMHEALHNHYTQKSLSLSPGK |
| 885 | Human IgG1 | apeaaggpsvflfppkpkdtlmisrtpevtcvvvdvkhedpevkfnwyvdgvevhn |
| | Fc variant | aktkpreeqynstyrvvsvltvlhqdwlngkeykckvsnkalaapiektiskakgq |
| | L234A/L235A/ | prepqvytlppsrdeltknqvsltclvkgfypsdiavewesngqpennykttppvl |
| | S267K/P329A | dsdgsfflyskltvdksrwqqgnvfscsvmhealhnhytqkslslspgk |
| | (LALASKPA) | |
| 886 | Fc | DKTHTCPPCPAPEAAGGPSVFLFPPKPKDTLMISRTPEVT |
| | LALASKPA | CVVVDVKHEDPEVKFNWYVDGVEVHNAKTKPREEQYN |
| | (L234A/ | STYRVVSVLTVLHQDWLNGKEYKCKVSNKALAAPIEKT |
| | L235A/S267K/ | ISKAKGQPREPQVYTLPPSREEMTKNQVSLTCLVKGFYP |
| | P329A) | SDIAVEWESNGQPENNYKTTPPVLDSDGSFFLYSKLTVD |
| | | KSRWQQGNVFSCSVMHEALHNHYTQKSLSLSPGK |
| 887 | Human IgG1 | apellggpsvflfppkpkdtlmisrtpevtcvvvavkhedpevkfnwyvdgvevhn |
| | Fc variant | aktkpreeqynstyrvvsvltvlhqdwlngkeykckvsnkalaapiektiskakgq |
| | D265A/P329A/ | prepqvytlppsrdeltknqvsltclvkgfypsdiavewesngqpennykttppvl |
| | S267K | dsdgsfflyskltvdksrwqqgnvfscsvmhealhnhytqkslslspgk |
| | (DAPASK) | |
| 888 | Human IgG1 | apellgapsvflfppkpkdtlmisrtpevtcvvvavshedpevkfnwyvdgvevhn |
| | Fc variant | aktkpreegynstyrvvsvltvlhqdwlngkeykckvsnkalaapiektiskakgq |
| | G237A/D265A/ | prepqvytlppsrdeltknqvsltclvkgfypsdiavewesngqpennykttppvl |
| | P329A | dsdgsfflyskltvdksrwqqgnvfscsvmhealhnhytqkslslspgk |
| | (GADAPA) | |
| 889 | Human IgG1 | apellgapsvflfppkpkdtlmisrtpevtcvvvavkhedpevkfnwyvdgvevhn |
| | Fc variant | aktkpreeqynstyrvvsvltvlhqdwlngkeykckvsnkalaapiektiskakgq |
| | G237A/D265A/ | prepqvytlppsrdeltknqvsltclvkgfypsdiavewesngqpennykttppvl |
| | P329A/S267K | dsdgsfflyskltvdksrwqqgnvfscsvmhealhnhytqkslslspgk |
| | (GADAPASK) | |
| 890 | Fc | DKTHTCPPCPAPELLGAPSVFLFPPKPKDTLMISRTPEVT |
| | GADAPASK | CVVVAVKHEDPEVKFNWYVDGVEVHNAKTKPREEQYN |
| | (G237A/ | STYRVVSVLTVLHQDWLNGKEYKCKVSNKALAAPIEKT |
| | D265A/ | ISKAKGQPREPQVYTLPPSRREEMKNQVSLTCLVKGFYP |
| | P329A/S267K) | SDIAVEWESNGQPENNYKTTPPVLDSDGSFFLYSKLTVD |
| | | KSRWQQGNVFSCSVMHEALHNHYTQKSLSLSPGK |
| 891 | Human IgG1 | apellggpsvflfppkpkdtlmisrtpevtcvvvavshedpevkfnwyvdgvevhn |
| | Fc variant | aktkpreeqyastyrvvsvltvlhqdwlngkeykckvsnkalaapiektiskakgq |
| | D265A/N297A/ | prepqvytlppsrdeltknqvsltclvkgfypsdiavewesngqpennykttppvl |
| | P329A | dsdgsfflyskltvdksrwqqgnvfscsvmhealhnhytqkslslspgk |
| | (DANAPA) | |
| 673 | NEG2042 LC | DVVMTQSPPSLLVTLGQPASISCRSSQSLLHSSGNTYLN |
| | | WLLQRPGQSPQPLIYLVSKLESGVPDRFSGSGSGTDFTLK |
| | | ISGVEAEDVGVYYCMQFTHYPYTFGQGTKLEIKRTVAAP |
| | | SVFIFPPSDEQLKSGTASVVCLLNNFYPREAKVQWKVDN |
| | | ALQSGNSQESVTEQDSKDSTYSLSSTLTLSKADYEKHKV |
| | | YACEVTHQGLSSPVTKSFNRGEC |
| 703 | NEG2042 HC | QVQLVQSGAEVQRPGASVKVSCKASGYIFTEYYMYWV |
| | | RQAPGQGLELVGRIDPEDGSIDYVEKFKKKVTLTADTSS |
| | | STAYMELSSLTSDDTAVYYCARGKFNYRFAYWGQGTL |
| | | VTVSSASTKGPSVFPLAPSSKSTSGGTAALGCLVKDYFPE |
| | | PVTVSWNSGALTSGVHTFPAVLQSSGLYSLSSVVTVPSS |
| | | SLGTQTYICNVNHKPSNTKVDKRVEPKSCDKTHTCPPCP |
| | | APELLGGPSVFLFPPKPKDTLMISRTPEVTCVVVAVSHED |
| | | PEVKFNWYVDGVEVHNAKTKPREEQYASTYRVVSVLT |
| | | VLHQDWLNGKEYKCKVSNKALAAPIEKTISKAKGQPRE |
| | | PQVYTLPPSREEMTKNQVSLTCLVKGFYPSDIAVEWESN |
| | | GQPENNYKTTPPVLDSDGSFFLYSKLTVDKSRWQQGNV |
| | | FSCSVMHEALHNHYTQKSLSLSPGGGGSGGGGSGGGGS |
| | | GGGGSQVQLVQSGGGVVQPGRSLRLSCKASGYTFTRYT |
| | | MHWVRQAPGKGLEWIGYINPSRGYTNYNQKVKDRFTIS |
| | | RDNSKNTAFLQMDSLRPEDTGVYFCARYYDDHYSLDY |
| | | WGQGTPVTVSSGGGGSGGGGSGGGGSGGGGSDIQMTQ |
| | | SPSSLSASVGDRVTITCSASSSVSYMNWYQQTPGKAPKR |
| | | WIYDTSKLASGVPSRFSGSGSGTDYTFTISSLQPEDIATY |
| | | YCQQWSSNPFTFGQGTKLQIT |
| 518 | NEG2043 LC | DIQMTQSPSSLSASVGDRVTITCHASQNIYVWLNWYQQ |
| | | KPGKAPKLLIYKASNLHTGVPSRFSGSGSGTDFTLTISSL |
| | | QPEDFATYYCQQGQTYPYTFGGGTKVEIKRTVAAPSVFI |
| | | FPPSDEQLKSGTASVVCLLNNFYPREAKVQWKVDNALQ |
| | | SGNSQESVTEQDSKDSTYSLSSTLTLSKADYEKHKVYAC |
| | | EVTHQGLSSPVTKSFNRGEC |
| 704 | NEG2043 HC | QVQLVQSGAEVKKPGASVKVSCKASGYTFTSYYIHWVR |
| | | QAPGQGLEWIGCIYPGNVNTNYNEKFKDRATLTVDTSIS |
| | | TAYMELSRLRSDDTAVYFCTRSHYGLDWNFDVWGQGT |
| | | TVTVSSASTKGPSVFPLAPSSKSTSGGTAALGCLVKDYFP |
| | | EPVTVSWNSGALTSGVHTFPAVLQSSGLYSLSSVVTVPS |
| | | SSLGTQTYICNVNHKPSNTKVDKRVEPKSCDKTHTCPPC |
| | | PAPELLGGPSVFLFPPKPKDTLMISRTPEVTCVVVAVSHE |
| | | DPEVKFNWYVDGVEVHNAKTKPREEQYASTYRVVSVL |
| | | TVLHQDWLNGKEYKCKVSNKALAAPIEKTISKAKGQPR |
| | | EPQVYTLPPSREEMTKNQVSLTCLVKGFYPSDIAVEWES |
| | | NGQPENNYKTTPPVLDSDGSFFLYSKLTVDKSRWQQGN |
| | | VFSCSVMHEALHNHYTQKSLSLSPGGGGSGGGGSGGGG |
| | | SGGGGSQVQLVQSGGGVVQPGRSLRLSCKASGYTFTRY |
| | | TMHWVRQAPGKGLEWIGYINPSRGYTNYNQKVKDRFTI |
| | | SRDNSKNTAFLQMDSLRPEDTGVYFCARYYDDHYSLDY |
| | | WGQGTPVTVSSGGGGSGGGGSGGGGSGGGGSDIQMTQ |
| | | SPSSLSASVGDRVTITCSASSSVSYMNWYQQTPGKAPKR |
| | | WIYDTSKLASGVPSRFSGSGSGTDYTFTISSLQPEDIATY |
| | | YCQQWSSNPFTFGQGTKLQIT |
| 705 | NEG2044 LC | DIVLTQPPSVSGAPGQRVTISCSGSSSNIVSNYVNWYQQL |
| | | PGTAPKLLIYDNNKRPSGVPDRFSGSKSGTSASLAITGLQ |
| | | SEDEADYYCQSYAIGSYSVVFGGGTKLTVLGQGTKVEIK |
| | | RTVAAPSVFIFPPSDEQLKSGTASVVCLLNNFYPREAKV |
| | | QWKVDNALQSGNSQESVTEQDSKDSTYSLSSTLTLSKA |
| | | DYEKHKVYACEVTHQGLSSPVTKSFNRGEC |
| 706 | NEG2044 HC | QVQLVESGGGLVQPGGSLRLSCAASGFTFSTYGMSWVR |
| | | QAPGKGLEWVSSIFYTGSSTYYADSVKGRFTISRDNSKN |
| | | TLYLQMNSLRAEDTAVYYCARIGYAGDSKYAIWGQGTL |
| | | VTVSSASTKGPSVFPLAPSSKSTSGGTAALGCLVKDYFPE |
| | | PVTVSWNSGALTSGVHTFPAVLQSSGLYSLSSVVTVPSS |
| | | SLGTQTYICNVNHKPSNTKVDKRVEPKSCDKTHTCPPCP |
| | | APELLGGPSVFLFPPKPKDTLMISRTPEVTCVVVAVSHED |
| | | PEVKFNWYVDGVEVHNAKTKPREEQYASTYRVVSVLT |
| | | VLHQDWLNGKEYKCKVSNKALAAPIEKTISKAKGQPRE |
| | | PQVYTLPPSREEMTKNQVSLTCLVKGFYPSDIAVEWESN |
| | | GQPENNYKTTPPVLDSDGSFFLYSKLTVDKSRWQQGNV |
| | | FSCSVMHEALHNHYTQKSLSLSPGGGGSGGGGSGGGGS |
| | | GGGGSQVQLVQSGGGVVQPGRSLRLSCKASGYTFTRYT |
| | | MHWVRQAPGKGLEWIGYINPSRGYTNYNQKVKDRFTIS |
| | | RDNSKNTAFLQMDSLRPEDTGVYFCARYYDDHYSLDY |
| | | WGQGTPVTVSSGGGGSGGGGSGGGGSGGGGSDIQMTQ |
| | | SPSSLSASVGDRVTITCSASSSVSYMNWYQQTPGKAPKR |
| | | WIYDTSKLASGVPSRFSGSGSGTDYTFTISSLQPEDIATY |
| | | YCQQWSSNPFTFGQGTKLQIT |
| 673 | NEG2050 LC | DVVMTQSPPSLLVTLGQPASISCRSSQSLLHSSGNTYLN |
| | | WLLQRPGQSPQPLIYLVSKLESGVPDRFSGSGSGTDFTLK |
| | | ISGVEAEDVGVYYCMQFTHYPYTFGQGTKLEIKRTVAAP |
| | | SVFIFPPSDEQLKSGTASVVCLLNNFYPREAKVQWKVDN |
| | | ALQSGNSQESVTEQDSKDSTYSLSSTLTLSKADYEKHKV |
| | | YACEVTHQGLSSPVTKSFNRGEC |
| 707 | NEG2050 HC | QVQLVQSGAEVQRPGASVKVSCKASGYIFTEYYMYWV |
| | | RQAPGQGLELVGRIDPEDGSIDYVEKFKKKVTLTADTSS |
| | | STAYMELSSLTSDDTAVYYCARGKFNYRFAYWGQGTL |
| | | VTVSSASTKGPSVFPLAPSSKSTSGGTAALGCLVKDYFPE |
| | | PVTVSWNSGALTSGVHTFPAVLQSSGLYSLSSVVTVPSS |
| | | SLGTQTYICNVNHKPSNTKVDKRVEPKSCDKTHTCPPCP |
| | | APELLGGPSVFLFPPKPKDTLMISRTPEVTCVVVAVSHED |
| | | PEVKFNWYVDGVEVHNAKTKPREEQYASTYRVVSVLT |
| | | VLHQDWLNGKEYKCKVSNKALAAPIEKTISKAKGQPRE |
| | | PQVYTLPPSREEMTKNQVSLTCLVKGFYPSDIAVEWESN |
| | | GQPENNYKTTPPVLDSDGSFFLYSKLTVDKSRWQQGNV |
| | | FSCSVMHEALHNHYTQKSLSLSPGGGGSGGGGSGGGGS |
| | | GGGGSEVQLVESGGGLVQPGGSLKLSCAASGFTFNKYA |
| | | MNWVRQAPGKCLEWVARIRSKYNNYATYYADSVKDRF |
| | | TISRDDSKNTAYLQMNNLKTEDTAVYYCVRHGNFGNSY |
| | | ISYWAYWGQGTLVTVSSGGGGSGGGGSGGGGSGGGGS |
| | | QTVVTQEPSLTVSPGGTVTLTCGSSTGAVTSGNYPNWV |
| | | QQKPGQAPRGLIGGTKFLAPGTPARFSGSLLGGKAALTL |
| | | SGVQPEDEAEYYCVLWYSNRWVFGCGTKLTVL |
| 673 | NEG2051 LC | DVVMTQSPPSLLVTLGQPASISCRSSQSLLHSSGNTYLN |
| | | WLLQRPGQSPQPLIYLVSKLESGVPDRFSGSGSGTDFTLK |
| | | ISGVEAEDVGVYYCMQFTHYPYTFGQGTKLEIKRTVAAP |
| | | SVFIFPPSDEQLKSGTASVVCLLNNFYPREAKVQWKVDN |
| | | ALQSGNSQESVTEQDSKDSTYSLSSTLTLSKADYEKHKV |
| | | YACEVTHQGLSSPVTKSFNRGEC |
| 708 | NEG2051 HC | QVQLVQSGAEVQRPGASVKVSCKASGYIFTEYYMYWV |
| | | RQAPGQGLELVGRIDPEDGSIDYVEKFKKKVTLTADTSS |
| | | STAYMELSSLTSDDTAVYYCARGKFNYRFAYWGQGTL |
| | | VTVSSASTKGPSVFPLAPSSKSTSGGTAALGCLVKDYFPE |
| | | PVTVSWNSGALTSGVHTFPAVLQSSGLYSLSSVVTVPSS |
| | | SLGTQTYICNVNHKPSNTKVDKRVEPKSCDKTHTCPPCP |
| | | APELLGGPSVFLFPPKPKDTLMISRTPEVTCVVVAVSHED |
| | | PEVKFNWYVDGVEVHNAKTKPREEQYASTYRVVSVLT |
| | | VLHQDWLNGKEYKCKVSNKALAAPIEKTISKAKGQPRE |
| | | PQVYTLPPSREEMTKNQVSLTCLVKGFYPSDIAVEWESN |
| | | GQPENNYKTTPPVLDSDGSFFLYSKLTVDKSRWQQGNV |
| | | FSCSVMHEALHNHYTQKSLSLSPGGGGSGGGGSGGGGS |
| | | GGGGSEVQLVESGGGLVQPGGSLKLSCAASGFTFNTYA |
| | | MNWVRQASGKCLEWVGRIRSKYNNYATYYADSVKDRF |
| | | TISRDDSKSTLYLQMNSLKTEDTAVYYCVRHGNFGNSY |
| | | VSWFAYWGQGTLVTVSSGGGGSGGGGSGGGGSQAVVT |
| | | QEPSLTVSPGGTVTLTCRSSTGAVTTSNYANWVQQKPG |
| | | QAPRGLIGGTNKRAPWTPARFSGSLLGDKAALTLSGAQP |
| | | EDEAEYFCALWYSNLWVFGCGTKLTVL |
| 518 | NEG2052 LC | DIQMTQSPSSLSASVGDRVTITCHASQNIYVWLNWYQQ |
| | | KPGKAPKLLIYKASNLHTGVPSRFSGSGSGTDFTLTISSL |
| | | QPEDFATYYCQQGQTYPYTFGGGTKVEIKRTVAAPSVFI |
| | | FPPSDEQLKSGTASVVCLLNNFYPREAKVQWKVDNALQ |
| | | SGNSQESVTEQDSKDSTYSLSSTLTLSKADYEKHKVYAC |
| | | EVTHQGLSSPVTKSFNRGEC |
| 709 | NEG2052 HC | QVQLVQSGAEVKKPGASVKVSCKASGYTFTSYYIHWVR |
| | | QAPGQGLEWIGCIYPGNVNTNYNEKFKDRATLTVDTSIS |
| | | TAYMELSRLRSDDTAVYFCTRSHYGLDWNFDVWGQGT |
| | | TVTVSSASTKGPSVFPLAPSSKSTSGGTAALGCLVKDYFP |
| | | EPVTVSWNSGALTSGVHTFPAVLQSSGLYSLSSVVTVPS |
| | | SSLGTQTYICNVNHKPSNTKVDKRVEPKSCDKTHTCPPC |
| | | PAPELLGGPSVFLFPPKPKDTLMISRTPEVTCVVVAVSHE |
| | | DPEVKFNWYVDGVEVHNAKTKPREEQYASTYRVVSVL |
| | | TVLHQDWLNGKEYKCKVSNKALAAPIEKTISKAKGQPR |
| | | EPQVYTLPPSREEMTKNQVSLTCLVKGFYPSDIAVEWES |
| | | NGQPENNYKTTPPVLDSDGSFFLYSKLTVDKSRWQQGN |
| | | VFSCSVMHEALHNHYTQKSLSLSPGGGGSGGGGSGGGG |
| | | SGGGGSEVQLVESGGGLVQPGGSLKLSCAASGFTFNKY |
| | | AMNWVRQAPGKCLEWVARIRSKYNNYATYYADSVKD |
| | | RFTISRDDSKNTAYLQMNNLKTEDTAVYYCVRHGNFGN |
| | | SYISYWAYWGQGTLVTVSSGGGGSGGGGSGGGGSGGG |
| | | GSQTVVTQEPSLTVSPGGTVTLTCGSSTGAVTSGNYPNW |
| | | VQQKPGQAPRGLIGGTKFLAPGTPARFSGSLLGGKAALT |
| | | LSGVQPEDEAEYYCVLWYSNRWVFGCGTKLTVL |
| 518 | NEG2053 LC | DIQMTQSPSSLSASVGDRVTITCHASQNIYVWLNWYQQ |
| | | KPGKAPKLLIYKASNLHTGVPSRFSGSGSGTDFTLTISSL |
| | | QPEDFATYYCQQGQTYPYTFGGGTKVEIKRTVAAPSVFI |
| | | FPPSDEQLKSGTASVVCLLNNFYPREAKVQWKVDNALQ |
| | | SGNSQESVTEQDSKDSTYSLSSTLTLSKADYEKHKVYAC |
| | | EVTHQGLSSPVTKSFNRGEC |
| 710 | NEG2053 HC | QVQLVQSGAEVKKPGASVKVSCKASGYTFTSYYIHWVR |
| | | QAPGQGLEWIGCIYPGNVNTNYNEKFKDRATLTVDTSIS |
| | | TAYMELSRLRSDDTAVYFCTRSHYGLDWNFDVWGQGT |
| | | TVTVSSASTKGPSVFPLAPSSKSTSGGTAALGCLVKDYFP |
| | | EPVTVSWNSGALTSGVHTFPAVLQSSGLYSLSSVVTVPS |
| | | SSLGTQTYICNVNHKPSNTKVDKRVEPKSCDKTHTCPPC |
| | | PAPELLGGPSVFLFPPKPKDTLMISRTPEVTCVVVAVSHE |
| | | DPEVKFNWYVDGVEVHNAKTKPREEQYASTYRVVSVL |
| | | TVLHQDWLNGKEYKCKVSNKALAAPIEKTISKAKGQPR |
| | | EPQVYTLPPSREEMTKNQVSLTCLVKGFYPSDIAVEWES |
| | | NGQPENNYKTTPPVLDSDGSFFLYSKLTVDKSRWQQGN |
| | | VFSCSVMHEALHNHYTQKSLSLSPGGGGSGGGGSGGGG |
| | | SGGGGSEVQLVESGGGLVQPGGSLKLSCAASGFTFNTYA |
| | | MNWVRQASGKCLEWVGRIRSKYNNYATYYADSVKDRF |
| | | TISRDDSKSTLYLQMNSLKTEDTAVYYCVRHGNFGNSY |
| | | VSWFAYWGQGTLVTVSSGGGGSGGGGSGGGGSQAVVT |
| | | QEPSLTVSPGGTVTLTCRSSTGAVTTSNYANWVQQKPG |
| | | QAPRGLIGGTNKRAPWTPARFSGSLLGDKAALTLSGAQP |
| | | EDEAEYFCALWYSNLWVFGCGTKLTVL |
| 673 | Construct 1 | DVVMTQSPPSLLVTLGQPASISCRSSQSLLHSSGNTYLN |
| | Light Chain | WLLQRPGQSPQPLIYLVSKLESGVPDRFSGSGSGTDFTLK |
| | | ISGVEAEDVGVYYCMQFTHYPYTFGQGTKLEIKRTVAAP |
| | | SVFIFPPSDEQLKSGTASVVCLLNNFYPREAKVQWKVDN |
| | | ALQSGNSQESVTEQDSKDSTYSLSSTLTLSKADYEKHKV |
| | | YACEVTHQGLSSPVTKSFNRGEC |
| 711 | Construct 1 | QVQLVQSGGGVVQPGRSLRLSCKASGYTFTRYTMHWV |
| | Heavy Chain | RQAPGKGLEWIGYINPSRGYTNYNQKVKDRFTISRDNSK |
| | | NTAFLQMDSLRPEDTGVYFCARYYDDHYSLDYWGQGT |
| | | PVTVSSGGGGSGGGGSGGGGSGGGGSDIQMTQSPSSLSA |
| | | SVGDRVTITCSASSSVSYMNWYQQTPGKAPKRWIYDTS |
| | | KLASGVPSRFSGSGSGTDYTFTISSLQPEDIATYYCQQWS |
| | | SNPFTFGQGTKLQITGGGGSGGGGSGGGGSGGGGSQVQ |
| | | LVQSGAEVQRPGASVKVSCKASGYIFTEYYMYWVRQAP |
| | | GQGLELVGRIDPEDGSIDYVEKFKKKVTLTADTSSSTAY |
| | | MELSSLTSDDTAVYYCARGKFNYRFAYWGQGTLVTVSS |
| | | ASTKGPSVFPLAPSSKSTSGGTAALGCLVKDYFPEPVTVS |
| | | WNSGALTSGVHTFPAVLQSSGLYSLSSVVTVPSSSLGTQ |
| | | TYICNVNHKPSNTKVDKRVEPKSCDKTHTCPPCPAPELL |
| | | GGPSVFLFPPKPKDTLMISRTPEVTCVVVDVSHEDPEVKF |
| | | NWYVDGVEVHNAKTKPREEQYNSTYRVVSVLTVLHQD |
| | | WLNGKEYKCKVSNKALPAPIEKTISKAKGQPREPQVYTL |
| | | PPSREEMTKNQVSLTCLVKGFYPSDIAVEWESNGQPENN |
| | | YKTTPPVLDSDGSFFLYSKLTVDKSRWQQGNVFSCSVM |
| | | HEALHNHYTQKSLSLSPGK |
| 518 | Construct 2 | DIQMTQSPSSLSASVGDRVTITCHASQNIYVWLNWYQQ |
| | Light Chain | KPGKAPKLLIYKASNLHTGVPSRFSGSGSGTDFTLTISSL |
| | | QPEDFATYYCQQGQTYPYTFGGGTKVEIKRTVAAPSVFI |
| | | FPPSDEQLKSGTASVVCLLNNFYPREAKVQWKVDNALQ |
| | | SGNSQESVTEQDSKDSTYSLSSTLTLSKADYEKHKVYAC |
| | | EVTHQGLSSPVTKSFNRGEC |
| 712 | Construct 2 | QVQLVQSGGGVVQPGRSLRLSCKASGYTFTRYTMHWV |
| | Heavy Chain | RQAPGKGLEWIGYINPSRGYTNYNQKVKDRFTISRDNSK |
| | | NTAFLQMDSLRPEDTGVYFCARYYDDHYSLDYWGQGT |
| | | PVTVSSGGGGSGGGGSGGGGSGGGGSDIQMTQSPSSLSA |
| | | SVGDRVTITCSASSSVSYMNWYQQTPGKAPKRWIYDTS |
| | | KLASGVPSRFSGSGSGTDYTFTISSLQPEDIATYYCQQWS |
| | | SNPFTFGQGTKLQITGGGGSGGGGSGGGGSGGGGSQVQ |
| | | LVQSGAEVKKPGASVKVSCKASGYTFTSYYIHWVRQAP |
| | | GQGLEWIGCIYPGNVNTNYNEKFKDRATLTVDTSISTAY |
| | | MELSRLRSDDTAVYFCTRSHYGLDWNFDVWGQGTTVT |
| | | VSSASTKGPSVFPLAPSSKSTSGGTAALGCLVKDYFPEPV |
| | | TVSWNSGALTSGVHTFPAVLQSSGLYSLSSVVTVPSSSL |
| | | GTQTYICNVNHKPSNTKVDKRVEPKSCDKTHTCPPCPAP |
| | | ELLGGPSVFLFPPKPKDTLMISRTPEVTCVVVDVSHEDPE |
| | | VKFNWYVDGVEVHNAKTKPREEQYNSTYRVVSVLTVL |
| | | HQDWLNGKEYKCKVSNKALPAPIEKTISKAKGQPREPQ |
| | | VYTLPPSREEMTKNQVSLTCLVKGFYPSDIAVEWESNGQ |
| | | PENNYKTTPPVLDSDGSFFLYSKLTVDKSRWQQGNVFSC |
| | | SVMHEALHNHYTQKSLSLSPGK |
| 673 | Construct 3 | DVVMTQSPPSLLVTLGQPASISCRSSQSLLHSSGNTYLN |
| | Light Chain | WLLQRPGQSPQPLIYLVSKLESGVPDRFSGSGSGTDFTLK |
| | | ISGVEAEDVGVYYCMQFTHYPYTFGQGTKLEIKRTVAAP |
| | | SVFIFPPSDEQLKSGTASVVCLLNNFYPREAKVQWKVDN |
| | | ALQSGNSQESVTEQDSKDSTYSLSSTLTLSKADYEKHKV |
| | | YACEVTHQGLSSPVTKSFNRGEC |
| 713 | Construct 3 | QVQLVQSGAEVQRPGASVKVSCKASGYIFTEYYMYWV |
| | Heavy Chain | RQAPGQGLELVGRIDPEDGSIDYVEKFKKKVTLTADTSS |
| | | STAYMELSSLTSDDTAVYYCARGKFNYRFAYWGQGTL |
| | | VTVSSASTKGPSVFPLAPSSKSTSGGTAALGCLVKDYFPE |
| | | PVTVSWNSGALTSGVHTFPAVLQSSGLYSLSSVVTVPSS |
| | | SLGTQTYICNVNHKPSNTKVDKRVEPKSCDKTHTCPPCP |
| | | APELLGGPSVFLFPPKPKDTLMISRTPEVTCVVVDVSHED |
| | | PEVKFNWYVDGVEVHNAKTKPREEQYNSTYRVVSVLT |
| | | VLHQDWLNGKEYKCKVSNKALPAPIEKTISKAKGQPREP |
| | | QVYTLPPSREEMTKNQVSLTCLVKGFYPSDIAVEWESNG |
| | | QPENNYKTTPPVLDSDGSFFLYSKLTVDKSRWQQGNVF |
| | | SCSVMHEALHNHYTQKSLSLSPGGGGSGGGGSGGGGSG |
| | | GGGSQVQLVQSGGGVVQPGRSLRLSCKASGYTFTRYTM |
| | | HWVRQAPGKGLEWIGYINPSRGYTNYNQKVKDRFTISR |
| | | DNSKNTAFLQMDSLRPEDTGVYFCARYYDDHYSLDYW |
| | | GQGTPVTVSSGGGGSGGGGSGGGGSGGGGSDIQMTQSP |
| | | SSLSASVGDRVTITCSASSSVSYMNWYQQTPGKAPKRWI |
| | | YDTSKLASGVPSRFSGSGSGTDYTFTISSLQPEDIATYYC |
| | | QQWSSNPFTFGQGTKLQIT |
| 518 | Construct 4 | DIQMTQSPSSLSASVGDRVTITCHASQNIYVWLNWYQQ |
| | Light Chain | KPGKAPKLLIYKASNLHTGVPSRFSGSGSGTDFTLTISSL |
| | | QPEDFATYYCQQGQTYPYTFGGGTKVEIKRTVAAPSVFI |
| | | FPPSDEQLKSGTASVVCLLNNFYPREAKVQWKVDNALQ |
| | | SGNSQESVTEQDSKDSTYSLSSTLTLSKADYEKHKVYAC |
| | | EVTHQGLSSPVTKSFNRGEC |
| 714 | Construct 4 | QVQLVQSGAEVKKPGASVKVSCKASGYTFTSYYIHWVR |
| | Heavy Chain | QAPGQGLEWIGCIYPGNVNTNYNEKFKDRATLTVDTSIS |
| | | TAYMELSRLRSDDTAVYFCTRSHYGLDWNFDVWGQGT |
| | | TVTVSSASTKGPSVFPLAPSSKSTSGGTAALGCLVKDYFP |
| | | EPVTVSWNSGALTSGVHTFPAVLQSSGLYSLSSVVTVPS |
| | | SSLGTQTYICNVNHKPSNTKVDKRVEPKSCDKTHTCPPC |
| | | PAPELLGGPSVFLFPPKPKDTLMISRTPEVTCVVVDVSHE |
| | | DPEVKFNWYVDGVEVHNAKTKPREEQYNSTYRVVSVL |
| | | TVLHQDWLNGKEYKCKVSNKALPAPIEKTISKAKGQPR |
| | | EPQVYTLPPSREEMTKNQVSLTCLVKGFYPSDIAVEWES |
| | | NGQPENNYKTTPPVLDSDGSFFLYSKLTVDKSRWQQGN |
| | | VFSCSVMHEALHNHYTQKSLSLSPGGGGSGGGGSGGGG |
| | | SGGGGSQVQLVQSGGGVVQPGRSLRLSCKASGYTFTRY |
| | | TMHWVRQAPGKGLEWIGYINPSRGYTNYNQKVKDRFTI |
| | | SRDNSKNTAFLQMDSLRPEDTGVYFCARYYDDHYSLDY |
| | | WGQGTPVTVSSGGGGSGGGGSGGGGSGGGGSDIQMTQ |
| | | SPSSLSASVGDRVTITCSASSSVSYMNWYQQTPGKAPKR |
| | | WIYDTSKLASGVPSRFSGSGSGTDYTFTISSLQPEDIATY |
| | | YCQQWSSNPFTFGQGTKLQIT |
| 673 | Construct 5 | DVVMTQSPPSLLVTLGQPASISCRSSQSLLHSSGNTYLN |
| | Light Chain | WLLQRPGQSPQPLIYLVSKLESGVPDRFSGSGSGTDFTLK |
| | | ISGVEAEDVGVYYCMQFTHYPYTFGQGTKLEIKRTVAAP |
| | | SVFIFPPSDEQLKSGTASVVCLLNNFYPREAKVQWKVDN |
| | | ALQSGNSQESVTEQDSKDSTYSLSSTLTLSKADYEKHKV |
| | | YACEVTHQGLSSPVTKSFNRGEC |
| 715 | Construct 5 | QVQLVQSGAEVQRPGASVKVSCKASGYIFTEYYMYWV |
| | Heavy Chain | RQAPGQGLELVGRIDPEDGSIDYVEKFKKKVTLTADTSS |
| | | STAYMELSSLTSDDTAVYYCARGKFNYRFAYWGQGTL |
| | | VTVSSASTKGPSVFPLAPSSKSTSGGTAALGCLVKDYFPE |
| | | PVTVSWNSGALTSGVHTFPAVLQSSGLYSLSSVVTVPSS |
| | | SLGTQTYICNVNHKPSNTKVDKRVEPKSCGGGGSGGGG |
| | | SQVQLVQSGGGVVQPGRSLRLSCKASGYTFTRYTMHW |
| | | VRQAPGKGLEWIGYINPSRGYTNYNQKVKDRFTISRDNS |
| | | KNTAFLQMDSLRPEDTGVYFCARYYDDHYSLDYWGQG |
| | | TPVTVSSGGGGSGGGGSGGGGSGGGGSDIQMTQSPSSLS |
| | | ASVGDRVTITCSASSSVSYMNWYQQTPGKAPKRWIYDT |
| | | SKLASGVPSRFSGSGSGTDYTFTISSLQPEDIATYYCQQW |
| | | SSNPFTFGQGTKLQITGGGGSGGGGSGGGGSGGGGSDKT |
| | | HTCPPCPAPELLGGPSVFLFPPKPKDTLMISRTPEVTCVV |
| | | VDVSHEDPEVKFNWYVDGVEVHNAKTKPREEQYNSTY |
| | | RVVSVLTVLHQDWLNGKEYKCKVSNKALPAPIEKTISK |
| | | AKGQPREPQVYTLPPSREEMTKNQVSLTCLVKGFYPSDI |
| | | AVEWESNGQPENNYKTTPPVLDSDGSFFLYSKLTVDKSR |
| | | WQQGNVFSCSVMHEALHNHYTQKSLSLSPGK |
| 518 | Construct 6 | DIQMTQSPSSLSASVGDRVTITCHASQNIYVWLNWYQQ |
| | Light Chain | KPGKAPKLLIYKASNLHTGVPSRFSGSGSGTDFTLTISSL |
| | | QPEDFATYYCQQGQTYPYTFGGGTKVEIKRTVAAPSVFI |
| | | FPPSDEQLKSGTASVVCLLNNFYPREAKVQWKVDNALQ |
| | | SGNSQESVTEQDSKDSTYSLSSTLTLSKADYEKHKVYAC |
| | | EVTHQGLSSPVTKSFNRGEC |
| 716 | Construct 6 | QVQLVQSGAEVKKPGASVKVSCKASGYTFTSYYIHWVR |
| | Heavy Chain | QAPGQGLEWIGCIYPGNVNTNYNEKFKDRATLTVDTSIS |
| | | TAYMELSRLRSDDTAVYFCTRSHYGLDWNFDVWGQGT |
| | | TVTVSSASTKGPSVFPLAPSSKSTSGGTAALGCLVKDYFP |
| | | EPVTVSWNSGALTSGVHTFPAVLQSSGLYSLSSVVTVPS |
| | | SSLGTQTYICNVNHKPSNTKVDKRVEPKSCGGGGSGGG |
| | | GSQVQLVQSGGGVVQPGRSLRLSCKASGYTFTRYTMH |
| | | WVRQAPGKGLEWIGYINPSRGYTNYNQKVKDRFTISRD |
| | | NSKNTAFLQMDSLRPEDTGVYFCARYYDDHYSLDYWG |
| | | QGTPVTVSSGGGGSGGGGSGGGGSGGGGSDIQMTQSPS |
| | | SLSASVGDRVTITCSASSSVSYMNWYQQTPGKAPKRWI |
| | | YDTSKLASGVPSRFSGSGSGTDYTFTISSLQPEDIATYYC |
| | | QQWSSNPFTFGQGTKLQITGGGGSGGGGSGGGGSGGGG |
| | | SDKTHTCPPCPAPELLGGPSVFLFPPKPKDTLMISRTPEVT |
| | | CVVVDVSHEDPEVKFNWYVDGVEVHNAKTKPREEQYN |
| | | STYRVVSVLTVLHQDWLNGKEYKCKVSNKALPAPIEKTI |
| | | SKAKGQPREPQVYTLPPSREEMTKNQVSLTCLVKGFYPS |
| | | DIAVEWESNGQPENNYKTTPPVLDSDGSFFLYSKLTVDK |
| | | SRWQQGNVFSCSVMHEALHNHYTQKSLSLSPGK |
| 673 | Construct 7 | DVVMTQSPPSLLVTLGQPASISCRSSQSLLHSSGNTYLN |
| | Light Chain | WLLQRPGQSPQPLIYLVSKLESGVPDRFSGSGSGTDFTLK |
| | | ISGVEAEDVGVYYCMQFTHYPYTFGQGTKLEIKRTVAAP |
| | | SVFIFPPSDEQLKSGTASVVCLLNNFYPREAKVQWKVDN |
| | | ALQSGNSQESVTEQDSKDSTYSLSSTLTLSKADYEKHKV |
| | | YACEVTHQGLSSPVTKSFNRGEC |
| 717 | Construct 7 | QVQLVQSGGGVVQPGRSLRLSCKASGYTFTRYTMHWV |
| | Heavy Chain | RQAPGKGLEWIGYINPSRGYTNYNQKVKDRFTISRDNSK |
| | | NTAFLQMDSLRPEDTGVYFCARYYDDHYSLDYWGQGT |
| | | PVTVSSGGGGSGGGGSGGGGSGGGGSDIQMTQSPSSLSA |
| | | SVGDRVTITCSASSSVSYMNWYQQTPGKAPKRWIYDTS |
| | | KLASGVPSRFSGSGSGTDYTFTISSLQPEDIATYYCQQWS |
| | | SNPFTFGQGTKLQITGGGGSDKTHTCPPCPAPELLGGPSV |
| | | FLFPPKPKDTLMISRTPEVTCVVVDVSHEDPEVKFNWYV |
| | | DGVEVHNAKTKPREEQYNSTYRVVSVLTVLHQDWLNG |
| | | KEYKCKVSNKALPAPIEKTISKAKGQPREPQVYTLPPSRE |
| | | EMTKNQVSLTCLVKGFYPSDIAVEWESNGQPENNYKTT |
| | | PPVLDSDGSFFLYSKLTVDKSRWQQGNVFSCSVMHEAL |
| | | HNHYTQKSLSLSPGGGGSGGGGSGGGGSGGGGSQVQLV |
| | | QSGAEVQRPGASVKVSCKASGYIFTEYYMYWVRQAPG |
| | | QGLELVGRIDPEDGSIDYVEKFKKKVTLTADTSSSTAYM |
| | | ELSSLTSDDTAVYYCARGKFNYRFAYWGQGTLVTVSSA |
| | | STKGPSVFPLAPSSKSTSGGTAALGCLVKDYFPEPVTVS |
| | | WNSGALTSGVHTFPAVLQSSGLYSLSSVVTVPSSSLGTQ |
| | | TYICNVNHKPSNTKVDKRVEPKSC |
| 518 | Construct 8 | DIQMTQSPSSLSASVGDRVTITCHASQNIYVWLNWYQQ |
| | Light Chain | KPGKAPKLLIYKASNLHTGVPSRFSGSGSGTDFTLTISSL |
| | | QPEDFATYYCQQGQTYPYTFGGGTKVEIKRTVAAPSVFI |
| | | FPPSDEQLKSGTASVVCLLNNFYPREAKVQWKVDNALQ |
| | | SGNSQESVTEQDSKDSTYSLSSTLTLSKADYEKHKVYAC |
| | | EVTHQGLSSPVTKSFNRGEC |
| 718 | Construct 8 | QVQLVQSGGGVVQPGRSLRLSCKASGYTFTRYTMHWV |
| | Heavy Chain | RQAPGKGLEWIGYINPSRGYTNYNQKVKDRFTISRDNSK |
| | | NTAFLQMDSLRPEDTGVYFCARYYDDHYSLDYWGQGT |
| | | PVTVSSGGGGSGGGGSGGGGSGGGGSDIQMTQSPSSLSA |
| | | SVGDRVTITCSASSSVSYMNWYQQTPGKAPKRWIYDTS |
| | | KLASGVPSRFSGSGSGTDYTFTISSLQPEDIATYYCQQWS |
| | | SNPFTFGQGTKLQITGGGGSDKTHTCPPCPAPELLGGPSV |
| | | FLFPPKPKDTLMISRTPEVTCVVVDVSHEDPEVKFNWYV |
| | | DGVEVHNAKTKPREEQYNSTYRVVSVLTVLHQDWLNG |
| | | KEYKCKVSNKALPAPIEKTISKAKGQPREPQVYTLPPSRE |
| | | EMTKNQVSLTCLVKGFYPSDIAVEWESNGQPENNYKTT |
| | | PPVLDSDGSFFLYSKLTVDKSRWQQGNVFSCSVMHEAL |
| | | HNHYTQKSLSLSPGGGGSGGGGSGGGGSGGGGSQVQLV |
| | | QSGAEVKKPGASVKVSCKASGYTFTSYYIHWVRQAPGQ |
| | | GLEWIGCIYPGNVNTNYNEKFKDRATLTVDTSISTAYME |
| | | LSRLRSDDTAVYFCTRSHYGLDWNFDVWGQGTTVTVSS |
| | | ASTKGPSVFPLAPSSKSTSGGTAALGCLVKDYFPEPVTVS |
| | | WNSGALTSGVHTFPAVLQSSGLYSLSSVVTVPSSSLGTQ |
| | | TYICNVNHKPSNTKVDKRVEPKSC |
| 673 | Construct 9 | DVVMTQSPPSLLVTLGQPASISCRSSQSLLHSSGNTYLN |
| | Light Chain | WLLQRPGQSPQPLIYLVSKLESGVPDRFSGSGSGTDFTLK |
| | | ISGVEAEDVGVYYCMQFTHYPYTFGQGTKLEIKRTVAAP |
| | | SVFIFPPSDEQLKSGTASVVCLLNNFYPREAKVQWKVDN |
| | | ALQSGNSQESVTEQDSKDSTYSLSSTLTLSKADYEKHKV |
| | | YACEVTHQGLSSPVTKSFNRGEC |
| 719 | Construct 9 | QVQLVQSGAEVQRPGASVKVSCKASGYIFTEYYMYWV |
| | Heavy Chain 1 | RQAPGQGLELVGRIDPEDGSIDYVEKFKKKVTLTADTSS |
| | | STAYMELSSLTSDDTAVYYCARGKFNYRFAYWGQGTL |
| | | VTVSSASTKGPSVFPLAPSSKSTSGGTAALGCLVKDYFPE |
| | | PVTVSWNSGALTSGVHTFPAVLQSSGLYSLSSVVTVPSS |
| | | SLGTQTYICNVNHKPSNTKVDKRVEPKSCDKTHTCPPCP |
| | | APELLGGPSVFLFPPKPKDTLMISRTPEVTCVVVDVSHED |
| | | PEVKFNWYVDGVEVHNAKTKPREEQYNSTYRVVSVLT |
| | | VLHQDWLNGKEYKCKVSNKALPAPIEKTISKAKGQPREP |
| | | QVCTLPPSREEMTKNQVSLSCAVKGFYPSDIAVEWESNG |
| | | QPENNYKTTPPVLDSDGSFFLVSKLTVDKSRWQQGNVF |
| | | SCSVMHEALHNRFTQKSLSLSPGK |
| 720 | Construct 9 | QVQLVQSGGGVVQPGRSLRLSCKASGYTFTRYTMHWV |
| | Heavy Chain 2 | RQAPGKGLEWIGYINPSRGYTNYNQKVKDRFTISRDNSK |
| | | NTAFLQMDSLRPEDTGVYFCARYYDDHYSLDYWGQGT |
| | | PVTVSSGGGGSGGGGSGGGGSGGGGSDIQMTQSPSSLSA |
| | | SVGDRVTITCSASSSVSYMNWYQQTPGKAPKRWIYDTS |
| | | KLASGVPSRFSGSGSGTDYTFTISSLQPEDIATYYCQQWS |
| | | SNPFTFGQGTKLQITGGGGSDKTHTCPPCPAPELLGGPSV |
| | | FLFPPKPKDTLMISRTPEVTCVVVDVSHEDPEVKFNWYV |
| | | DGVEVHNAKTKPREEQYNSTYRVVSVLTVLHQDWLNG |
| | | KEYKCKVSNKALPAPIEKTISKAKGQPREPQVYTLPPCRE |
| | | EMTKNQVSLWCLVKGFYPSDIAVEWESNGQPENNYKTT |
| | | PPVLDSDGSFFLYSKLTVDKSRWQQGNVFSCSVMHEAL |
| | | HNHYTQKSLSLSPGK |
| 518 | Construct 10 | DIQMTQSPSSLSASVGDRVTITCHASQNIYVWLNWYQQ |
| | Light Chain | KPGKAPKLLIYKASNLHTGVPSRFSGSGSGTDFTLTISSL |
| | | QPEDFATYYCQQGQTYPYTFGGGTKVEIKRTVAAPSVFI |
| | | FPPSDEQLKSGTASVVCLLNNFYPREAKVQWKVDNALQ |
| | | SGNSQESVTEQDSKDSTYSLSSTLTLSKADYEKHKVYAC |
| | | EVTHQGLSSPVTKSFNRGEC |
| 721 | Construct 10 | QVQLVQSGAEVKKPGASVKVSCKASGYTFTSYYIHWVR |
| | Heavy Chain 1 | QAPGQGLEWIGCIYPGNVNTNYNEKFKDRATLTVDTSIS |
| | | TAYMELSRLRSDDTAVYFCTRSHYGLDWNFDVWGQGT |
| | | TVTVSSASTKGPSVFPLAPSSKSTSGGTAALGCLVKDYFP |
| | | EPVTVSWNSGALTSGVHTFPAVLQSSGLYSLSSVVTVPS |
| | | SSLGTQTYICNVNHKPSNTKVDKRVEPKSCDKTHTCPPC |
| | | PAPELLGGPSVFLFPPKPKDTLMISRTPEVTCVVVDVSHE |
| | | DPEVKFNWYVDGVEVHNAKTKPREEQYNSTYRVVSVL |
| | | TVLHQDWLNGKEYKCKVSNKALPAPIEKTISKAKGQPR |
| | | EPQVCTLPPSREEMTKNQVSLSCAVKGFYPSDIAVEWES |
| | | NGQPENNYKTTPPVLDSDGSFFLVSKLTVDKSRWQQGN |
| | | VFSCSVMHEALHNRFTQKSLSLSPGK |
| 720 | Construct 10 | QVQLVQSGGGVVQPGRSLRLSCKASGYTFTRYTMHWV |
| | Heavy Chain 2 | RQAPGKGLEWIGYINPSRGYTNYNQKVKDRFTISRDNSK |
| | | NTAFLQMDSLRPEDTGVYFCARYYDDHYSLDYWGQGT |
| | | PVTVSSGGGGSGGGGSGGGGSGGGGSDIQMTQSPSSLSA |
| | | SVGDRVTITCSASSSVSYMNWYQQTPGKAPKRWIYDTS |
| | | KLASGVPSRFSGSGSGTDYTFTISSLQPEDIATYYCQQWS |
| | | SNPFTFGQGTKLQITGGGGSDKTHTCPPCPAPELLGGPSV |
| | | FLFPPKPKDTLMISRTPEVTCVVVDVSHEDPEVKFNWYV |
| | | DGVEVHNAKTKPREEQYNSTYRVVSVLTVLHQDWLNG |
| | | KEYKCKVSNKALPAPIEKTISKAKGQPREPQVYTLPPCRE |
| | | EMTKNQVSLWCLVKGFYPSDIAVEWESNGQPENNYKTT |
| | | PPVLDSDGSFFLYSKLTVDKSRWQQGNVFSCSVMHEAL |
| | | HNHYTQKSLSLSPGK |
| 722 | Construct 11 | DKTHTCPPCPAPELLGGPSVFLFPPKPKDTLMISRTPEVT |
| | Heavy Chain 1 | CVVVDVSHEDPEVKFNWYVDGVEVHNAKTKPREEQYN |
| | | STYRVVSVLTVLHQDWLNGKEYKCKVSNKALPAPIEKTI |
| | | SKAKGQPREPQVCTLPPSREEMTKNQVSLSCAVKGFYPS |
| | | DIAVEWESNGQPENNYKTTPPVLDSDGSFFLVSKLTVDK |
| | | SRWQQGNVFSCSVMHEALHNRFTQKSLSLSPGK |
| 720 | Construct 11 | QVQLVQSGGGVVQPGRSLRLSCKASGYTFTRYTMHWV |
| | Heavy Chain 2 | RQAPGKGLEWIGYINPSRGYTNYNQKVKDRFTISRDNSK |
| | | NTAFLQMDSLRPEDTGVYFCARYYDDHYSLDYWGQGT |
| | | PVTVSSGGGGSGGGGSGGGGSGGGGSDIQMTQSPSSLSA |
| | | SVGDRVTITCSASSSVSYMNWYQQTPGKAPKRWIYDTS |
| | | KLASGVPSRFSGSGSGTDYTFTISSLQPEDIATYYCQQWS |
| | | SNPFTFGQGTKLQITGGGGSDKTHTCPPCPAPELLGGPSV |
| | | FLFPPKPKDTLMISRTPEVTCVVVDVSHEDPEVKFNWYV |
| | | DGVEVHNAKTKPREEQYNSTYRVVSVLTVLHQDWLNG |
| | | KEYKCKVSNKALPAPIEKTISKAKGQPREPQVYTLPPCRE |
| | | EMTKNQVSLWCLVKGFYPSDIAVEWESNGQPENNYKTT |
| | | PPVLDSDGSFFLYSKLTVDKSRWQQGNVFSCSVMHEAL |
| | | HNHYTQKSLSLSPGK |
| 673 | Construct 12 | DVVMTQSPPSLLVTLGQPASISCRSSQSLLHSSGNTYLN |
| | Light Chain | WLLQRPGQSPQPLIYLVSKLESGVPDRFSGSGSGTDFTLK |
| | | ISGVEAEDVGVYYCMQFTHYPYTFGQGTKLEIKRTVAAP |
| | | SVFIFPPSDEQLKSGTASVVCLLNNFYPREAKVQWKVDN |
| | | ALQSGNSQESVTEQDSKDSTYSLSSTLTLSKADYEKHKV |
| | | YACEVTHQGLSSPVTKSFNRGEC |
| 719 | Construct 12 | QVQLVQSGAEVQRPGASVKVSCKASGYIFTEYYMYWV |
| | Heavy Chain 1 | RQAPGQGLELVGRIDPEDGSIDYVEKFKKKVTLTADTSS |
| | | STAYMELSSLTSDDTAVYYCARGKFNYRFAYWGQGTL |
| | | VTVSSASTKGPSVFPLAPSSKSTSGGTAALGCLVKDYFPE |
| | | PVTVSWNSGALTSGVHTFPAVLQSSGLYSLSSVVTVPSS |
| | | SLGTQTYICNVNHKPSNTKVDKRVEPKSCDKTHTCPPCP |
| | | APELLGGPSVFLFPPKPKDTLMISRTPEVTCVVVDVSHED |
| | | PEVKFNWYVDGVEVHNAKTKPREEQYNSTYRVVSVLT |
| | | VLHQDWLNGKEYKCKVSNKALPAPIEKTISKAKGQPREP |
| | | QVCTLPPSREEMTKNQVSLSCAVKGFYPSDIAVEWESNG |
| | | QPENNYKTTPPVLDSDGSFFLVSKLTVDKSRWQQGNVF |
| | | SCSVMHEALHNRFTQKSLSLSPGK |
| 723 | Construct 12 | QVQLVQSGGGVVQPGRSLRLSCKASGYTFTRYTMHWV |
| | Heavy Chain 2 | RQAPGKGLEWIGYINPSRGYTNYNQKVKDRFTISRDNSK |
| | | NTAFLQMDSLRPEDTGVYFCARYYDDHYSLDYWGQGT |
| | | PVTVSSGGGGSGGGGSGGGGSGGGGSDIQMTQSPSSLSA |
| | | SVGDRVTITCSASSSVSYMNWYQQTPGKAPKRWIYDTS |
| | | KLASGVPSRFSGSGSGTDYTFTISSLQPEDIATYYCQQWS |
| | | SNPFTFGQGTKLQITGGGGSDKTHTCPPCPAPELLGGPSV |
| | | FLFPPKPKDTLMISRTPEVTCVVVDVSHEDPEVKFNWYV |
| | | DGVEVHNAKTKPREEQYNSTYRVVSVLTVLHQDWLNG |
| | | KEYKCKVSNKALPAPIEKTISKAKGQPREPQVYTLPPCRE |
| | | EMTKNQVSLWCLVKGFYPSDIAVEWESNGQPENNYKTT |
| | | PPVLDSDGSFFLYSKLTVDKSRWQQGNVFSCSVMHEAL |
| | | HNHYTQKSLSLSPGGGGGSGGGGSGGGGSEEDPCACES |
| | | LVKFQAKVEGLLQALTRKLEAVSKRLAILENTVV |
| 518 | Construct 13 | DIQMTQSPSSLSASVGDRVTITCHASQNIYVWLNWYQQ |
| | Light Chain | KPGKAPKLLIYKASNLHTGVPSRFSGSGSGTDFTLTISSL |
| | | QPEDFATYYCQQGQTYPYTFGGGTKVEIKRTVAAPSVFI |
| | | FPPSDEQLKSGTASVVCLLNNFYPREAKVQWKVDNALQ |
| | | SGNSQESVTEQDSKDSTYSLSSTLTLSKADYEKHKVYAC |
| | | EVTHQGLSSPVTKSFNRGEC |
| 721 | Construct 13 | QVQLVQSGAEVKKPGASVKVSCKASGYTFTSYYIHWVR |
| | Heavy Chain 1 | QAPGQGLEWIGCIYPGNVNTNYNEKFKDRATLTVDTSIS |
| | | TAYMELSRLRSDDTAVYFCTRSHYGLDWNFDVWGQGT |
| | | TVTVSSASTKGPSVFPLAPSSKSTSGGTAALGCLVKDYFP |
| | | EPVTVSWNSGALTSGVHTFPAVLQSSGLYSLSSVVTVPS |
| | | SSLGTQTYICNVNHKPSNTKVDKRVEPKSCDKTHTCPPC |
| | | PAPELLGGPSVFLFPPKPKDTLMISRTPEVTCVVVDVSHE |
| | | DPEVKFNWYVDGVEVHNAKTKPREEQYNSTYRVVSVL |
| | | TVLHQDWLNGKEYKCKVSNKALPAPIEKTISKAKGQPR |
| | | EPQVCTLPPSREEMTKNQVSLSCAVKGFYPSDIAVEWES |
| | | NGQPENNYKTTPPVLDSDGSFFLVSKLTVDKSRWQQGN |
| | | VFSCSVMHEALHNRFTQKSLSLSPGK |
| 723 | Construct 13 | QVQLVQSGGGVVQPGRSLRLSCKASGYTFTRYTMHWV |
| | Heavy Chain 2 | RQAPGKGLEWIGYINPSRGYTNYNQKVKDRFTISRDNSK |
| | | NTAFLQMDSLRPEDTGVYFCARYYDDHYSLDYWGQGT |
| | | PVTVSSGGGGSGGGGSGGGGSGGGGSDIQMTQSPSSLSA |
| | | SVGDRVTITCSASSSVSYMNWYQQTPGKAPKRWIYDTS |
| | | KLASGVPSRFSGSGSGTDYTFTISSLQPEDIATYYCQQWS |
| | | SNPFTFGQGTKLQITGGGGSDKTHTCPPCPAPELLGGPSV |
| | | FLFPPKPKDTLMISRTPEVTCVVVDVSHEDPEVKFNWYV |
| | | DGVEVHNAKTKPREEQYNSTYRVVSVLTVLHQDWLNG |
| | | KEYKCKVSNKALPAPIEKTISKAKGQPREPQVYTLPPCRE |
| | | EMTKNQVSLWCLVKGFYPSDIAVEWESNGQPENNYKTT |
| | | PPVLDSDGSFFLYSKLTVDKSRWQQGNVFSCSVMHEAL |
| | | HNHYTQKSLSLSPGGGGGSGGGGSGGGGSEEDPCACES |
| | | LVKFQAKVEGLLQALTRKLEAVSKRLAILENTVV |
| 722 | Construct 14 | DKTHTCPPCPAPELLGGPSVFLFPPKPKDTLMISRTPEVT |
| | Heavy Chain 1 | CVVVDVSHEDPEVKFNWYVDGVEVHNAKTKPREEQYN |
| | | STYRVVSVLTVLHQDWLNGKEYKCKVSNKALPAPIEKTI |
| | | SKAKGQPREPQVCTLPPSREEMTKNQVSLSCAVKGFYPS |
| | | DIAVEWESNGQPENNYKTTPPVLDSDGSFFLVSKLTVDK |
| | | SRWQQGNVFSCSVMHEALHNRFTQKSLSLSPGK |
| 723 | Construct 14 | QVQLVQSGGGVVQPGRSLRLSCKASGYTFTRYTMHWV |
| | Heavy Chain 2 | RQAPGKGLEWIGYINPSRGYTNYNQKVKDRFTISRDNSK |
| | | NTAFLQMDSLRPEDTGVYFCARYYDDHYSLDYWGQGT |
| | | PVTVSSGGGGSGGGGSGGGGSGGGGSDIQMTQSPSSLSA |
| | | SVGDRVTITCSASSSVSYMNWYQQTPGKAPKRWIYDTS |
| | | KLASGVPSRFSGSGSGTDYTFTISSLQPEDIATYYCQQWS |
| | | SNPFTFGQGTKLQITGGGGSDKTHTCPPCPAPELLGGPSV |
| | | FLFPPKPKDTLMISRTPEVTCVVVDVSHEDPEVKFNWYV |
| | | DGVEVHNAKTKPREEQYNSTYRVVSVLTVLHQDWLNG |
| | | KEYKCKVSNKALPAPIEKTISKAKGQPREPQVYTLPPCRE |
| | | EMTKNQVSLWCLVKGFYPSDIAVEWESNGQPENNYKTT |
| | | PPVLDSDGSFFLYSKLTVDKSRWQQGNVFSCSVMHEAL |
| | | HNHYTQKSLSLSPGGGGGSGGGGSGGGGSEEDPCACES |
| | | LVKFQAKVEGLLQALTRKLEAVSKRLAILENTVV |
| 673 | Construct 15 | DVVMTQSPPSLLVTLGQPASISCRSSQSLLHSSGNTYLN |
| | Light Chain | WLLQRPGQSPQPLIYLVSKLESGVPDRFSGSGSGTDFTLK |
| | | ISGVEAEDVGVYYCMQFTHYPYTFGQGTKLEIKRTVAAP |
| | | SVFIFPPSDEQLKSGTASVVCLLNNFYPREAKVQWKVDN |
| | | ALQSGNSQESVTEQDSKDSTYSLSSTLTLSKADYEKHKV |
| | | YACEVTHQGLSSPVTKSFNRGEC |
| 719 | Construct 15 | QVQLVQSGAEVQRPGASVKVSCKASGYIFTEYYMYWV |
| | Heavy Chain 1 | RQAPGQGLELVGRIDPEDGSIDYVEKFKKKVTLTADTSS |
| | | STAYMELSSLTSDDTAVYYCARGKFNYRFAYWGQGTL |
| | | VTVSSASTKGPSVFPLAPSSKSTSGGTAALGCLVKDYFPE |
| | | PVTVSWNSGALTSGVHTFPAVLQSSGLYSLSSVVTVPSS |
| | | SLGTQTYICNVNHKPSNTKVDKRVEPKSCDKTHTCPPCP |
| | | APELLGGPSVFLFPPKPKDTLMISRTPEVTCVVVDVSHED |
| | | PEVKFNWYVDGVEVHNAKTKPREEQYNSTYRVVSVLT |
| | | VLHQDWLNGKEYKCKVSNKALPAPIEKTISKAKGQPREP |
| | | QVCTLPPSREEMTKNQVSLSCAVKGFYPSDIAVEWESNG |
| | | QPENNYKTTPPVLDSDGSFFLVSKLTVDKSRWQQGNVF |
| | | SCSVMHEALHNRFTQKSLSLSPGK |
| 724 | Construct 15 | QVQLVQSGGGVVQPGRSLRLSCKASGYTFTRYTMHWV |
| | Heavy Chain 2 | RQAPGKGLEWIGYINPSRGYTNYNQKVKDRFTISRDNSK |
| | | NTAFLQMDSLRPEDTGVYFCARYYDDHYSLDYWGQGT |
| | | PVTVSSGGGGSGGGGSGGGGSGGGGSDIQMTQSPSSLSA |
| | | SVGDRVTITCSASSSVSYMNWYQQTPGKAPKRWIYDTS |
| | | KLASGVPSRFSGSGSGTDYTFTISSLQPEDIATYYCQQWS |
| | | SNPFTFGQGTKLQITGGGGSDKTHTCPPCPAPELLGGPSV |
| | | FLFPPKPKDTLMISRTPEVTCVVVDVSHEDPEVKFNWYV |
| | | DGVEVHNAKTKPREEQYNSTYRVVSVLTVLHQDWLNG |
| | | KEYKCKVSNKALPAPIEKTISKAKGQPREPQVYTLPPCRE |
| | | EMTKNQVSLWCLVKGFYPSDIAVEWESNGQPENNYKTT |
| | | PPVLDSDGSFFLYSKLTVDKSRWQQGNVFSCSVMHEAL |
| | | HNHYTQKSLSLSPGGGGGSDLGPQMLRELQETNAALQD |
| | | VRELLRQQVREITFLKNTVMECDACGMQQ |
| 518 | Construct 16 | DIQMTQSPSSLSASVGDRVTITCHASQNIYVWLNWYQQ |
| | Light Chain | KPGKAPKLLIYKASNLHTGVPSRFSGSGSGTDFTLTISSL |
| | | QPEDFATYYCQQGQTYPYTFGGGTKVEIKRTVAAPSVFI |
| | | FPPSDEQLKSGTASVVCLLNNFYPREAKVQWKVDNALQ |
| | | SGNSQESVTEQDSKDSTYSLSSTLTLSKADYEKHKVYAC |
| | | EVTHQGLSSPVTKSFNRGEC |
| 721 | Construct 16 | QVQLVQSGAEVKKPGASVKVSCKASGYTFTSYYIHWVR |
| | Heavy Chain 1 | QAPGQGLEWIGCIYPGNVNTNYNEKFKDRATLTVDTSIS |
| | | TAYMELSRLRSDDTAVYFCTRSHYGLDWNFDVWGQGT |
| | | TVTVSSASTKGPSVFPLAPSSKSTSGGTAALGCLVKDYFP |
| | | EPVTVSWNSGALTSGVHTFPAVLQSSGLYSLSSVVTVPS |
| | | SSLGTQTYICNVNHKPSNTKVDKRVEPKSCDKTHTCPPC |
| | | PAPELLGGPSVFLFPPKPKDTLMISRTPEVTCVVVDVSHE |
| | | DPEVKFNWYVDGVEVHNAKTKPREEQYNSTYRVVSVL |
| | | TVLHQDWLNGKEYKCKVSNKALPAPIEKTISKAKGQPR |
| | | EPQVCTLPPSREEMTKNQVSLSCAVKGFYPSDIAVEWES |
| | | NGQPENNYKTTPPVLDSDGSFFLVSKLTVDKSRWQQGN |
| | | VFSCSVMHEALHNRFTQKSLSLSPGK |
| 724 | Construct 16 | QVQLVQSGGGVVQPGRSLRLSCKASGYTFTRYTMHWV |
| | Heavy Chain 2 | RQAPGKGLEWIGYINPSRGYTNYNQKVKDRFTISRDNSK |
| | | NTAFLQMDSLRPEDTGVYFCARYYDDHYSLDYWGQGT |
| | | PVTVSSGGGGSGGGGSGGGGSGGGGSDIQMTQSPSSLSA |
| | | SVGDRVTITCSASSSVSYMNWYQQTPGKAPKRWIYDTS |
| | | KLASGVPSRFSGSGSGTDYTFTISSLQPEDIATYYCQQWS |
| | | SNPFTFGQGTKLQITGGGGSDKTHTCPPCPAPELLGGPSV |
| | | FLFPPKPKDTLMISRTPEVTCVVVDVSHEDPEVKFNWYV |
| | | DGVEVHNAKTKPREEQYNSTYRVVSVLTVLHQDWLNG |
| | | KEYKCKVSNKALPAPIEKTISKAKGQPREPQVYTLPPCRE |
| | | EMTKNQVSLWCLVKGFYPSDIAVEWESNGQPENNYKTT |
| | | PPVLDSDGSFFLYSKLTVDKSRWQQGNVFSCSVMHEAL |
| | | HNHYTQKSLSLSPGGGGGSDLGPQMLRELQETNAALQD |
| | | VRELLRQQVREITFLKNTVMECDACGMQQ |
| 722 | Construct 17 | DKTHTCPPCPAPELLGGPSVFLFPPKPKDTLMISRTPEVT |
| | Heavy Chain 1 | CVVVDVSHEDPEVKFNWYVDGVEVHNAKTKPREEQYN |
| | | STYRVVSVLTVLHQDWLNGKEYKCKVSNKALPAPIEKTI |
| | | SKAKGQPREPQVCTLPPSREEMTKNQVSLSCAVKGFYPS |
| | | DIAVEWESNGQPENNYKTTPPVLDSDGSFFLVSKLTVDK |
| | | SRWQQGNVFSCSVMHEALHNRFTQKSLSLSPGK |
| 724 | Construct 17 | QVQLVQSGGGVVQPGRSLRLSCKASGYTFTRYTMHWV |
| | Heavy Chain 2 | RQAPGKGLEWIGYINPSRGYTNYNQKVKDRFTISRDNSK |
| | | NTAFLQMDSLRPEDTGVYFCARYYDDHYSLDYWGQGT |
| | | PVTVSSGGGGSGGGGSGGGGSGGGGSDIQMTQSPSSLSA |
| | | SVGDRVTITCSASSSVSYMNWYQQTPGKAPKRWIYDTS |
| | | KLASGVPSRFSGSGSGTDYTFTISSLQPEDIATYYCQQWS |
| | | SNPFTFGQGTKLQITGGGGSDKTHTCPPCPAPELLGGPSV |
| | | FLFPPKPKDTLMISRTPEVTCVVVDVSHEDPEVKFNWYV |
| | | DGVEVHNAKTKPREEQYNSTYRVVSVLTVLHQDWLNG |
| | | KEYKCKVSNKALPAPIEKTISKAKGQPREPQVYTLPPCRE |
| | | EMTKNQVSLWCLVKGFYPSDIAVEWESNGQPENNYKTT |
| | | PPVLDSDGSFFLYSKLTVDKSRWQQGNVFSCSVMHEAL |
| | | HNHYTQKSLSLSPGGGGGSDLGPQMLRELQETNAALQD |
| | | VRELLRQQVREITFLKNTVMECDACGMQQ |
| SEQ ID | Anti-CD3 | QVQLVESGGGLVQPGGSLRLSCAASGFTFSTYGMSWVR |
| NO: 726 | (4)/anti-CD28 | QAPGKGLEWVSSIFYTGSSTYYADSVKGRFTISRDNSKN |
| | (2) bispecific | TLYLQMNSLRAEDTAVYYCARIGYAGDSKYAIWGQGTL |
| | heavy chain | VTVSSASTKGPSVFPLAPSSKSTSGGTAALGCLVKDYFPE |
| | | PVTVSWNSGALTSGVHTFPAVLQSSGLYSLSSVVTVPSS |
| | | SLGTQTYICNVNHKPSNTKVDKRVEPKSCDKTHTCPPCP |
| | | APEAAGGPSVFLFPPKPKDTLMISRTPEVTCVVVDVKHE |
| | | DPEVKFNWYVDGVEVHNAKTKPREEQYNSTYRVVSVL |
| | | TVLHQDWLNGKEYKCKVSNKALAAPIEKTISKAKGQPR |
| | | EPQVYTLPPSREEMTKNQVSLTCLVKGFYPSDIAVEWES |
| | | NGQPENNYKTTPPVLDSDGSFFLYSKLTVDKSRWQQGN |
| | | VFSCSVMHEALHNHYTQKSLSLSPGGGGGSGGGGSGGG |
| | | GSGGGGSEVKLVESGGDLVQPGDSLTLSCVASGFTFSKQ |
| | | GMHWIRQAPKKGLEWIAMIYYDSSKMYYADTVKGRFTI |
| | | SRDNSKNTLYLEMNSLRSEDTAMYYCASFWWDLDFDH |
| | | WGQGVMVTVSSGGGGSGGGGSGGGGSGGGGSDILVTQ |
| | | TPVSLPVSLGGHVSISCRSSQSLVRSEGTTYFNWYLQKPG |
| | | QSPQLLIYRVSNRFSGVPDRFSGSGSGTDFTLKISRVEPED |
| | | LGVYYCLQSSHFPWTFGGGTKLELK |
| SEQ ID | Anti-CD3 | EVQLVESGGGLVQPGGSLRLSCAASGFTFSTYGMSWVR |
| NO: 1416 | (4)/anti-CD28 | QAPGKGLEWVSSIFYTGSSTYYADSVKGRFTISRDNSKN |
| | (2) bispecific | TLYLQMNSLRAEDTAVYYCARIGYAGDSKYAIWGQGTL |
| | alternative | VTVSSASTKGPSVFPLAPSSKSTSGGTAALGCLVKDYFPE |
| | heavy chain | PVTVSWNSGALTSGVHTFPAVLQSSGLYSLSSVVTVPSS |
| | | SLGTQTYICNVNHKPSNTKVDKRVEPKSCDKTHTCPPCP |
| | | APEAAGGPSVFLFPPKPKDTLMISRTPEVTCVVVDVKHE |
| | | DPEVKFNWYVDGVEVHNAKTKPREEQYNSTYRVVSVL |
| | | TVLHQDWLNGKEYKCKVSNKALAAPIEKTISKAKGQPR |
| | | EPQVYTLPPSREEMTKNQVSLTCLVKGFYPSDIAVEWES |
| | | NGQPENNYKTTPPVLDSDGSFFLYSKLTVDKSRWQQGN |
| | | VFSCSVMHEALHNHYTQKSLSLSPGGGGGSGGGGSGGG |
| | | GSGGGGSEVKLVESGGDLVQPGDSLTLSCVASGFTFSKQ |
| | | GMHWIRQAPKKGLEWIAMIYYDSSKMYYADTVKGRFTI |
| | | SRDNSKNTLYLEMNSLRSEDTAMYYCASFWWDLDFDH |
| | | WGQGVMVTVSSGGGGSGGGGSGGGGSGGGGSDILVTQ |
| | | TPVSLPVSLGGHVSISCRSSQSLVRSEGTTYFNWYLQKPG |
| | | QSPQLLIYRVSNRFSGVPDRFSGSGSGTDFTLKISRVEPED |
| | | LGVYYCLQSSHFPWTFGGGTKLELK |
| SEQ ID | Anti-CD3 | DIVLTQPPSVSGAPGQRVTISCSGSSSNIVSNYVNWYQQL |
| NO: 728 | (4)/anti-CD28 | PGTAPKLLIYDNNKRPSGVPDRFSGSKSGTSASLAITGLQ |
| | (2) bispecific | SEDEADYYCQSYAIGSYSVVFGGGTKLTVLGQGTKVEIK |
| | light chain 1 | RTVAAPSVFIFPPSDEQLKSGTASVVCLLNNFYPREAKV |
| | | QWKVDNALQSGNSQESVTEQDSKDSTYSLSSTLTLSKA |
| | | DYEKHKVYACEVTHQGLSSPVTKSFNRGEC |
| SEQ ID | Anti-CD3 | DIVLTQPPSVSGAPGQRVTISCSGSSSNIVSNYVNWYQQL |
| NO: 730 | (4)/anti-CD28 | PGTAPKLLIYDNNKRPSGVPDRFSGSKSGTSASLAITGLQ |
| | (2) bispecific | SEDEADYYCQSYAIGSYSVVFGGGTKLTVLGQPKAAPSV |
| | light chain 2 | TLFPPSSEELQANKATLVCLISDFYPGAVTVAWKADSSP |
| | | VKAGVETTTPSKQSNNKYAASSYLSLTPEQWKSHRSYS |
| | | CQVTHEGSTVEKTVAPTECS |
| 892 | Anti-CD3 | DIVLTQPPSVSGAPGQRVTISCSGSSSNIVSNYVNWYQQL |
| | (2)/anti-CD28 | PGTAPKLLIYDNNKRPSGVPDRFSGSKSGTSASLAITGLQ |
| | (2) light chain | SEDEADYYCQSYAIGSYSVVFGGGTKLTVLGQGTKVEIK |
| | | RTVAAPSVFIFPPSDEQLKSGTASVVCLLNNFYPREAKV |
| | | QWKVDNALQSGNSQESVTEQDSKDSTYSLSSTLTLSKA |
| | | DYEKHKVYACEVTHQGLSSPVTKSFNRGEC |
| 893 | Anti-CD3 | QVQLVESGGGLVQPGGSLRLSCAASGFTFSTYGMSWVR |
| | (2)/anti-CD28 | QAPGKGLEWVSSIFYTGSSTYYADSVKGRFTISRDNSKN |
| | (2) heavy chain | TLYLQMNSLRAEDTAVYYCARIGYAGDSKYAIWGQGTL |
| | | VTVSSASTKGPSVFPLAPSSKSTSGGTAALGCLVKDYFPE |
| | | PVTVSWNSGALTSGVHTFPAVLQSSGLYSLSSVVTVPSS |
| | | SLGTQTYICNVNHKPSNTKVDKRVEPKSCDKTHTCPPCP |
| | | APEAAGGPSVFLFPPKPKDTLMISRTPEVTCVVVDVKHE |
| | | DPEVKFNWYVDGVEVHNAKTKPREEQYNSTYRVVSVL |
| | | TVLHQDWLNGKEYKCKVSNKALAAPIEKTISKAKGQPR |
| | | EPQVYTLPPSREEMTKNQVSLTCLVKGFYPSDIAVEWES |
| | | NGQPENNYKTTPPVLDSDGSFFLYSKLTVDKSRWQQGN |
| | | VFSCSVMHEALHNHYTQKSLSLSPGGGGGSGGGGSGGG |
| | | GSGGGGSEVQLVESGGGLVQPGGSLKLSCAASGFTFNK |
| | | YAMNWVRQAPGKCLEWVARIRSKYNNYATYYADSVK |
| | | DRFTISRDDSKNTAYLQMNNLKTEDTAVYYCVRHGNFG |
| | | NSYISYWAYWGQGTLVTVSSGGGGSGGGGSGGGGSGG |
| | | GGSQTVVTQEPSLTVSPGGTVTLTCGSSTGAVTSGNYPN |
| | | WVQQKPGQAPRGLIGGTKFLAPGTPARFSGSLLGGKAA |
| | | LTLSGVQPEDEAEYYCVLWYSNRWVFGCGTKLTVL |
| SEQ ID | Anti-CD3 | EVQLVESGGGLVQPGGSLRLSCAASGFTFSTYGMSWVR |
| NO: 1417 | (2)/anti-CD28 | QAPGKGLEWVSSIFYTGSSTYYADSVKGRFTISRDNSKN |
| | (2) alternative | TLYLQMNSLRAEDTAVYYCARIGYAGDSKYAIWGQGTL |
| | heavy chain | VTVSSASTKGPSVFPLAPSSKSTSGGTAALGCLVKDYFPE |
| | | PVTVSWNSGALTSGVHTFPAVLQSSGLYSLSSVVTVPSS |
| | | SLGTQTYICNVNHKPSNTKVDKRVEPKSCDKTHTCPPCP |
| | | APEAAGGPSVFLFPPKPKDTLMISRTPEVTCVVVDVKHE |
| | | DPEVKFNWYVDGVEVHNAKTKPREEQYNSTYRVVSVL |
| | | TVLHQDWLNGKEYKCKVSNKALAAPIEKTISKAKGQPR |
| | | EPQVYTLPPSREEMTKNQVSLTCLVKGFYPSDIAVEWES |
| | | NGQPENNYKTTPPVLDSDGSFFLYSKLTVDKSRWQQGN |
| | | VFSCSVMHEALHNHYTQKSLSLSPGGGGGSGGGGSGGG |
| | | GSGGGGSEVQLVESGGGLVQPGGSLKLSCAASGFTFNK |
| | | YAMNWVRQAPGKCLEWVARIRSKYNNYATYYADSVK |
| | | DRFTISRDDSKNTAYLQMNNLKTEDTAVYYCVRHGNFG |
| | | NSYISYWAYWGQGTLVTVSSGGGGSGGGGSGGGGSGG |
| | | GGSQTVVTQEPSLTVSPGGTVTLTCGSSTGAVTSGNYPN |
| | | WVQQKPGQAPRGLIGGTKFLAPGTPARFSGSLLGGKAA |
| | | LTLSGVQPEDEAEYYCVLWYSNRWVFGCGTKLTVL |
| 894 | Anti-CD3 | DIQMTQSPSSLSASVGDRVTITCHASQNIYVWLNWYQQ |
| | (4)/anti-CD28 | KPGKAPKLLIYKASNLHTGVPSRFSGSGSGTDFTLTISSL |
| | (1) light chain | QPEDFATYYCQQGQTYPYTFGGGTKVEIKRTVAAPSVFI |
| | | FPPSDEQLKSGTASVVCLLNNFYPREAKVQWKVDNALQ |
| | | SGNSQESVTEQDSKDSTYSLSSTLTLSKADYEKHKVYAC |
| | | EVTHQGLSSPVTKSFNRGEC |
| 895 | Anti-CD3 | QVQLVQSGAEVKKPGASVKVSCKASGYTFTSYYIHWVR |
| | (4)/anti-CD28 | QAPGQGLEWIGCIYPGNVNTNYNEKFKDRATLTVDTSIS |
| | (1) heavy chain | TAYMELSRLRSDDTAVYFCTRSHYGLDWNFDVWGQGT |
| | | TVTVSSASTKGPSVFPLAPSSKSTSGGTAALGCLVKDYFP |
| | | EPVTVSWNSGALTSGVHTFPAVLQSSGLYSLSSVVTVPS |
| | | SSLGTQTYICNVNHKPSNTKVDKRVEPKSCDKTHTCPPC |
| | | PAPEAAGGPSVFLFPPKPKDTLMISRTPEVTCVVVDVKH |
| | | EDPEVKFNWYVDGVEVHNAKTKPREEQYNSTYRVVSV |
| | | LTVLHQDWLNGKEYKCKVSNKALAAPIEKTISKAKGQP |
| | | REPQVYTLPPSREEMTKNQVSLTCLVKGFYPSDIAVEWE |
| | | SNGQPENNYKTTPPVLDSDGSFFLYSKLTVDKSRWQQG |
| | | NVFSCSVMHEALHNHYTQKSLSLSPGGGSEVKLVESGG |
| | | DLVQPGDSLTLSCVASGFTFSKQGMHWIRQAPKKGLEW |
| | | IAMIYYDSSKMYYADTVKGRFTISRDNSKNTLYLEMNSL |
| | | RSEDTAMYYCASFWWDLDFDHWGQGVMVTVSSGGGG |
| | | SGGGGSGGGGSGGGGSDILVTQTPVSLPVSLGGHVSISC |
| | | RSSQSLVRSEGTTYFNWYLQKPGQSPQLLIYRVSNRFSG |
| | | VPDRFSGSGSGTDFTLKISRVEPEDLGVYYCLQSSHFPWT |
| | | FGGGTKLELK |

In some embodiments, the multispecific binding molecule comprises a bispecific antibody. In some embodiments, the bispecific antibody is configured in any one of the schema provided in FIGS. 37A-37B, FIG. 48A-48B, FIG. 49A-49C,and FIG. 50A-50B. In some embodiments, the bispecific antibody is monovalent or bivalent. In some embodiments, the bispecific antibody comprises an Fc region. In some embodiments, the Fc region of the bispecific antibody is silenced.

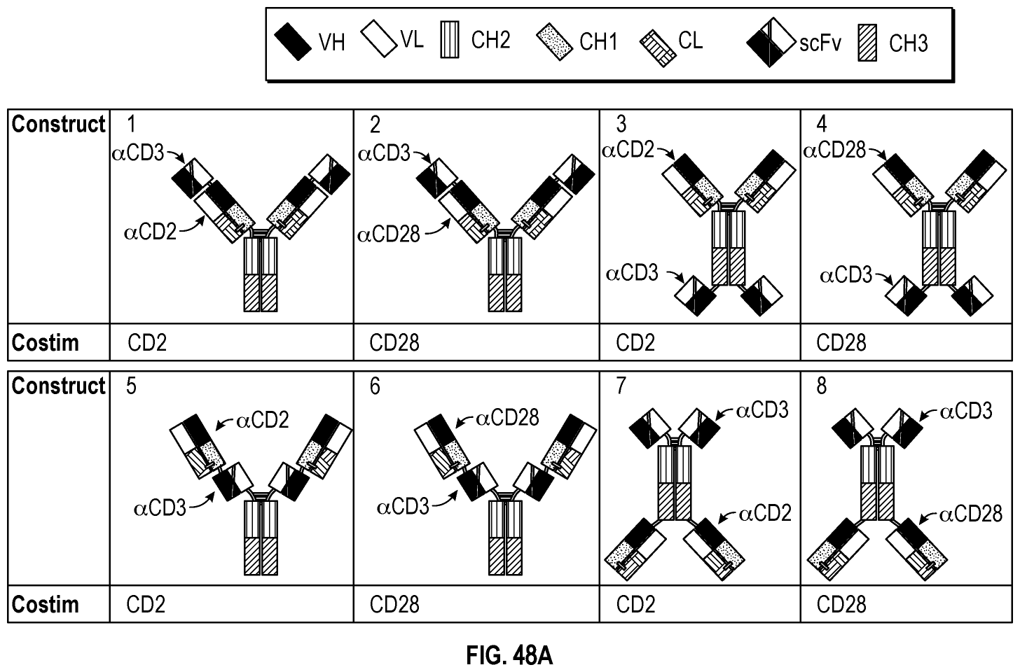

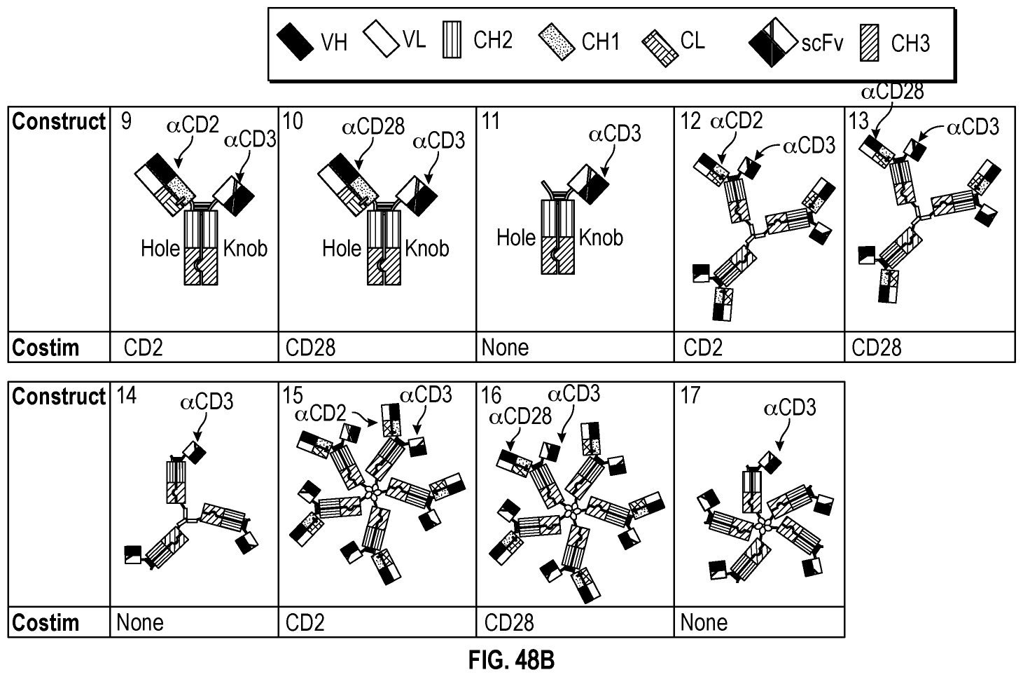

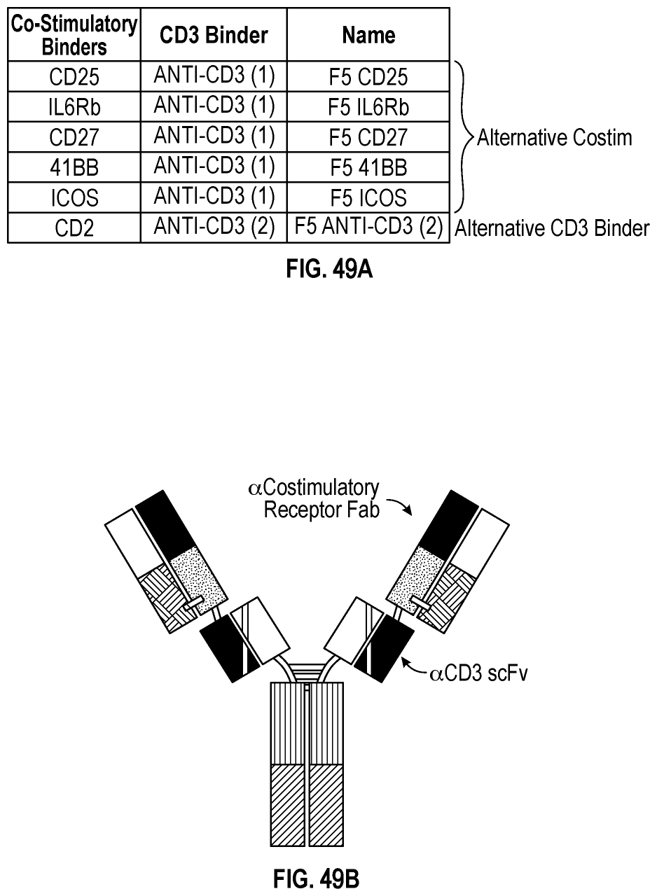

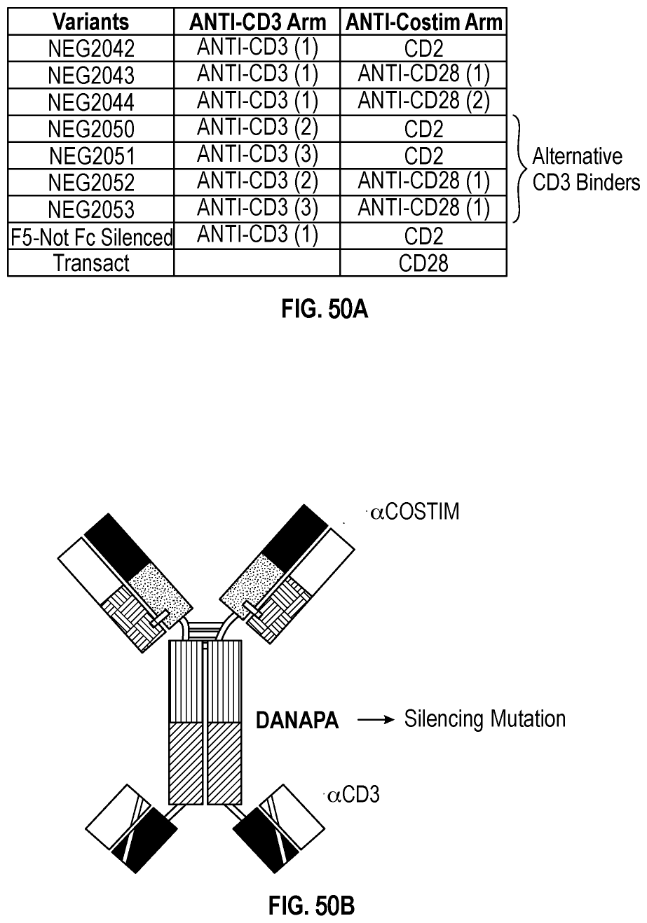

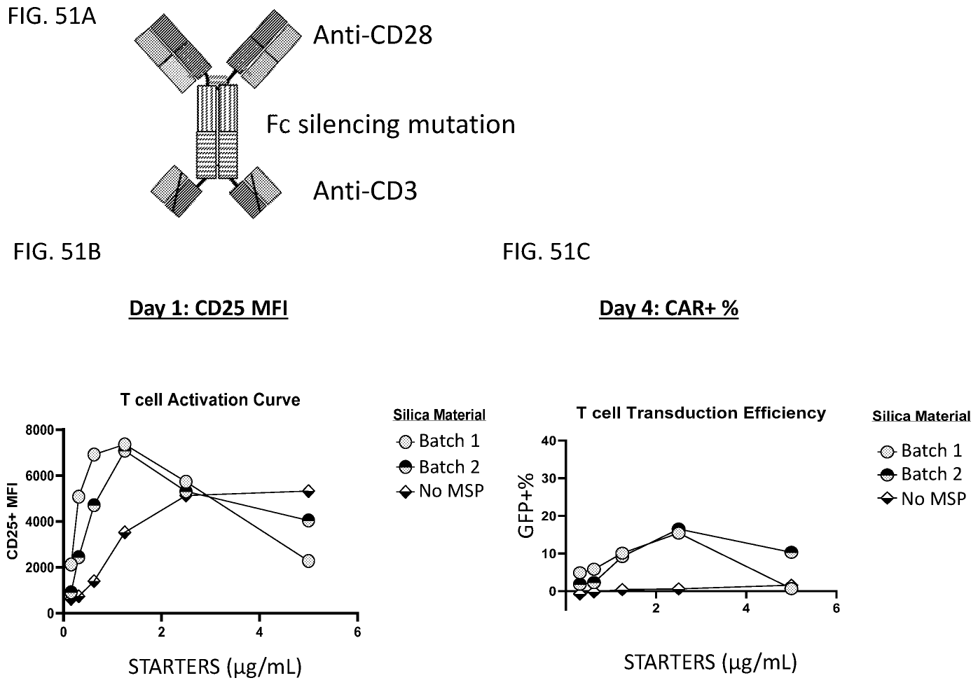

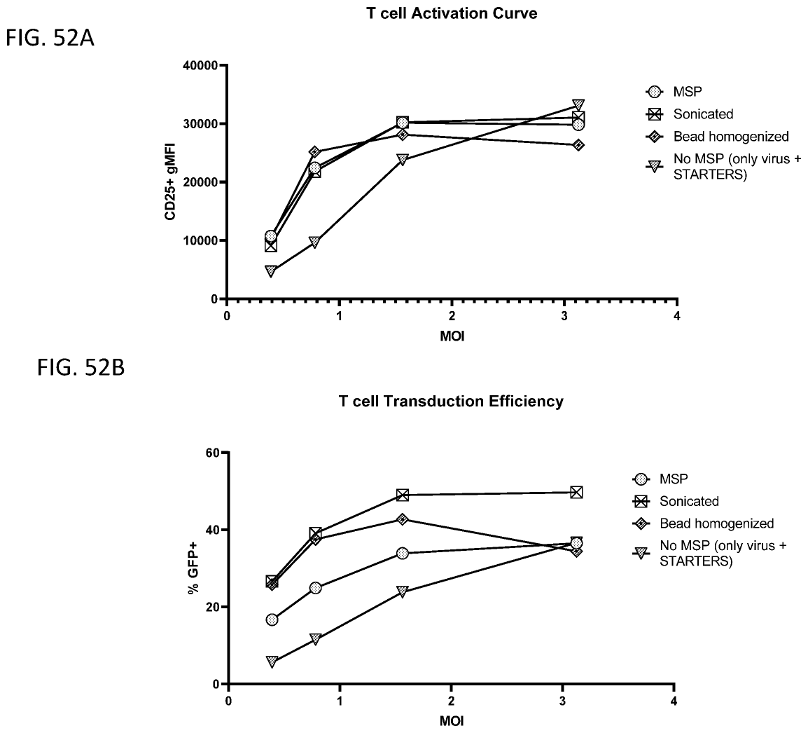

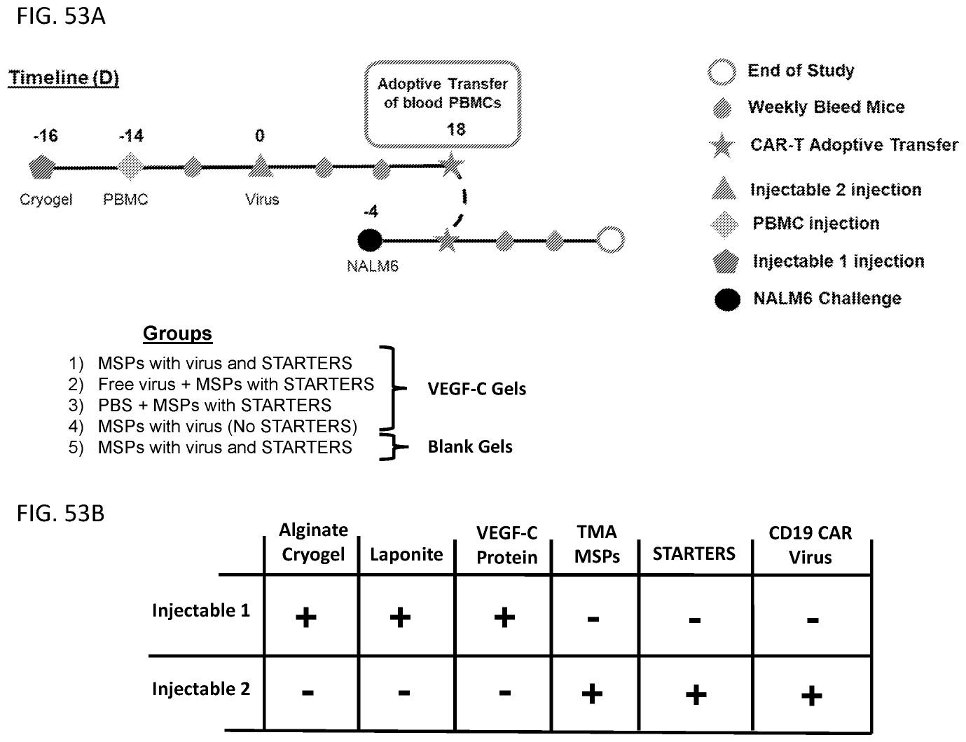

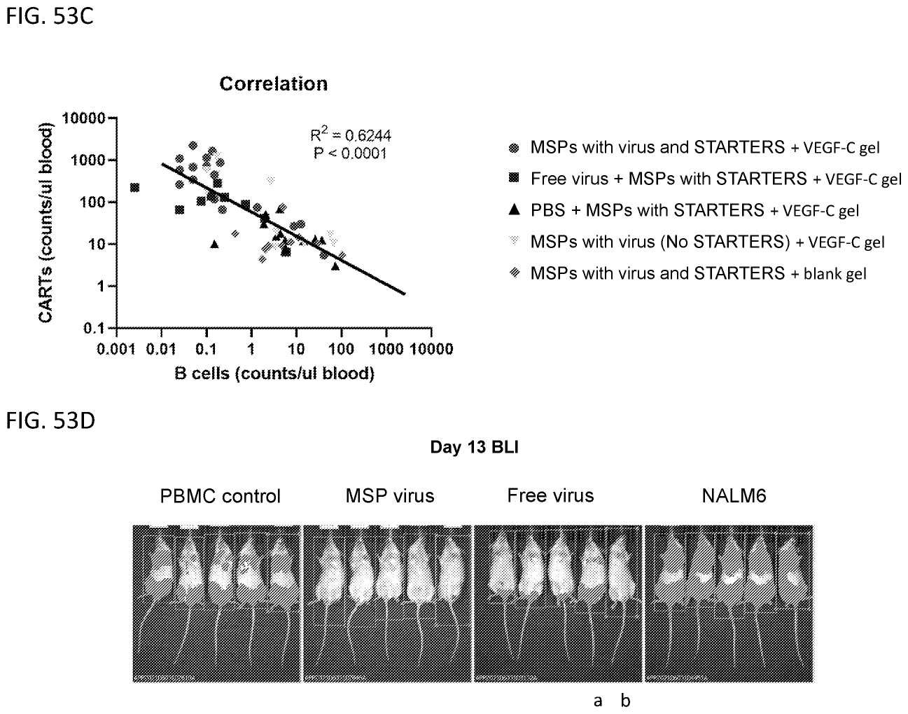

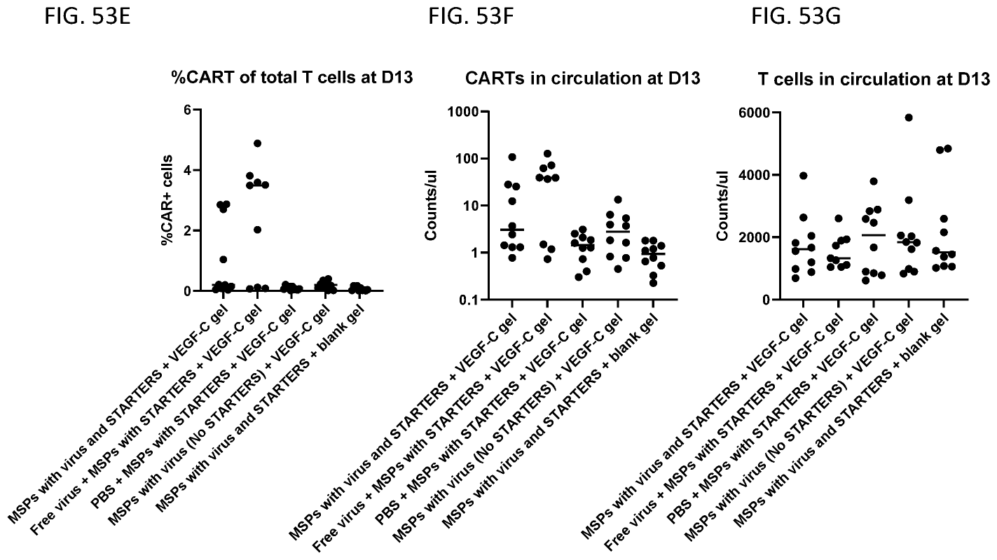

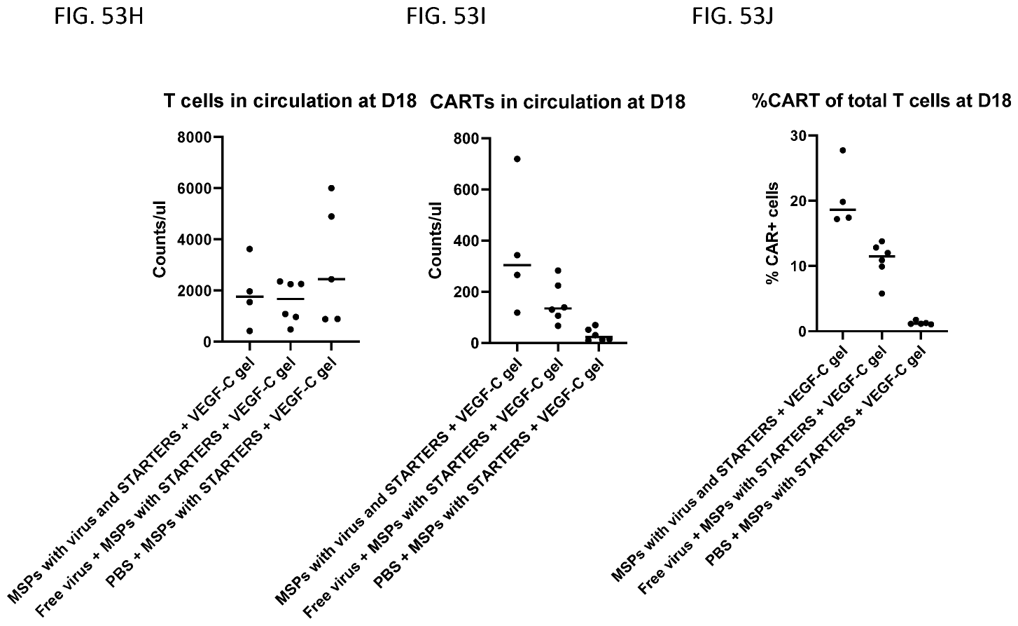

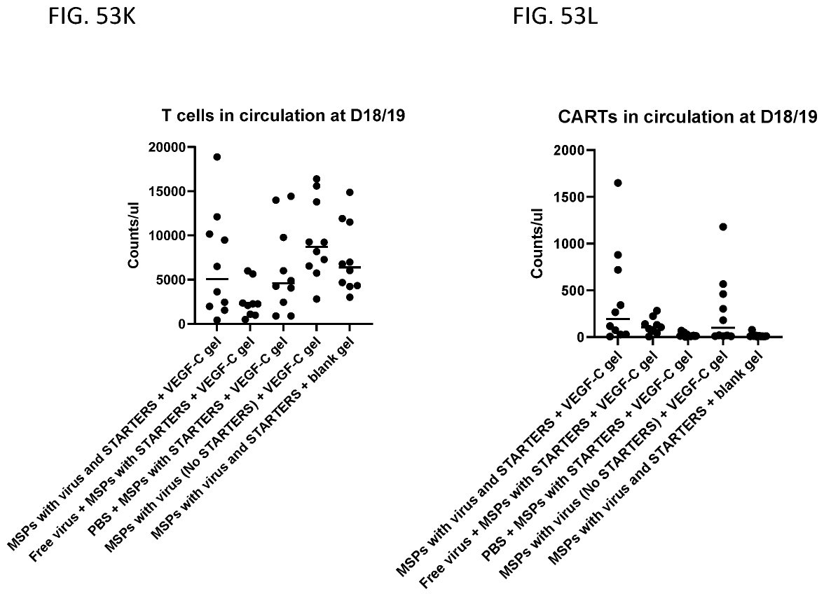

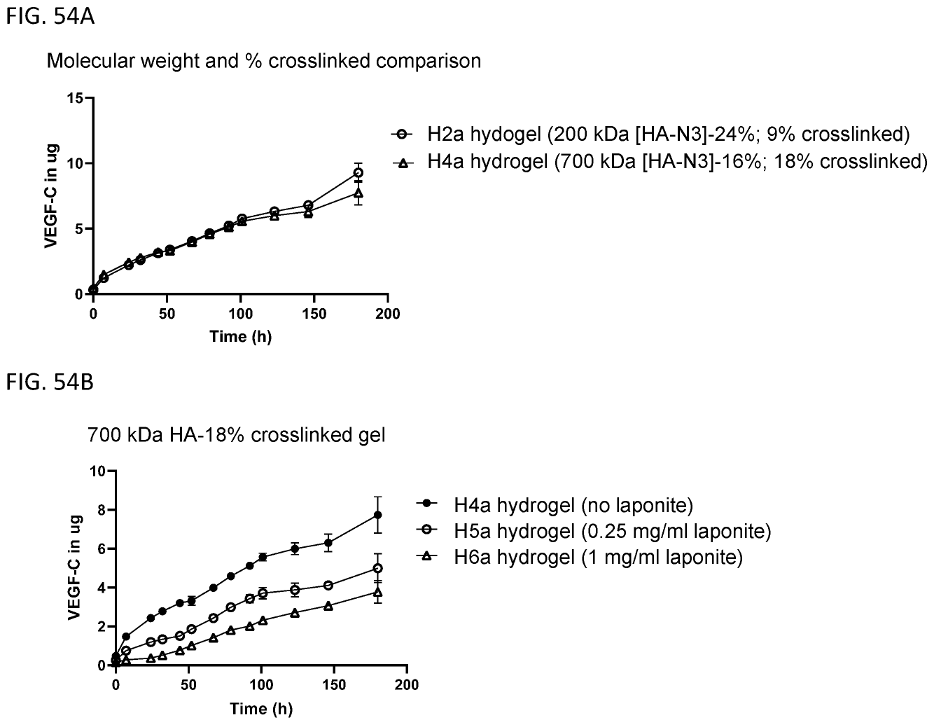

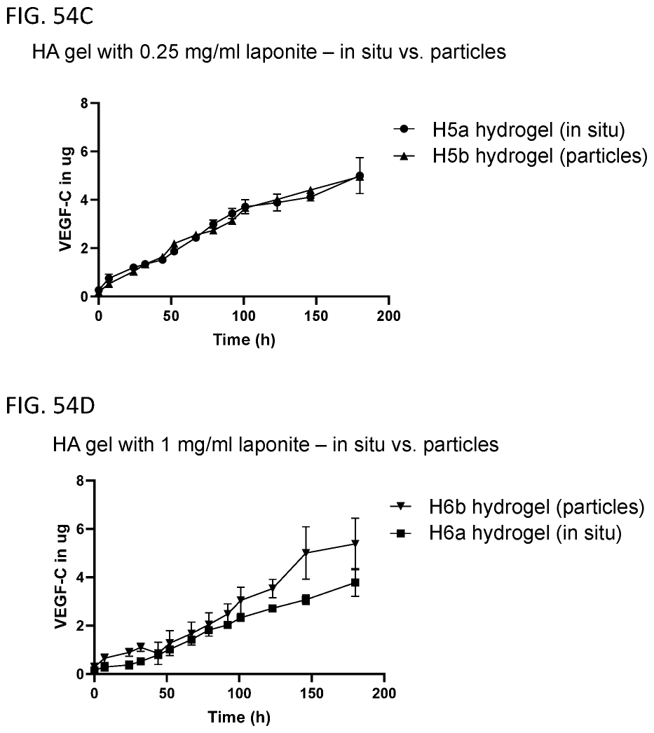

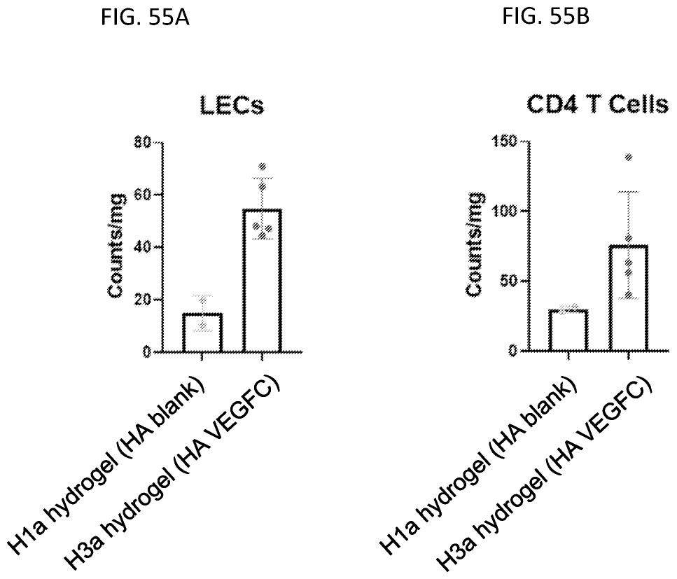

In some embodiments, the multispecific binding molecule comprises a plurality of bispecific antibodies. In some embodiments, one or more of the plurality of bispecific antibodies is monovalent. In some embodiments, one or more of the plurality of bispecific antibodies comprises an Fc region. In some embodiments, the Fc region of the one or more of the plurality of bispecific antibodies is silenced. In some embodiments, one or more of the plurality of bispecific antibodies are conjugated together into a multimer. In some embodiments, the multimer is configured in any one of the multispecific schema provided in FIG. 37B and FIG. 48B.

In some embodiments, a multispecific binding molecule described herein comprises an Fc region, e.g., wherein the Fc region is Fc silent. In some embodiments, the Fc region comprises a mutation at one or more of (e.g., all of) D265, N297, and P329, numbered according to the Eu numbering system. In some embodiments, the Fc region comprises the mutations D265A, N297A, and P329A (DANAPA), numbered according to the Eu numbering system.

In some embodiments, a multispecific binding molecule described herein comprises a first binding domain and a second binding domain. For instance, the first binding domain may be an anti-CD3 binding domain and the second binding domain may be a costimulatory molecule binding domain, or the first binding domain may be a costimulatory molecule binding domain and the second binding domain may be an anti-CD3 binding domain. In some embodiments, the costimulatory molecule binding domain binds to CD2, CD28, CD25, CD27, IL6Rb, ICOS, or 41BB. In some embodiments, the costimulatory molecule binding domain activates CD2, CD28, CD25, CD27, IL6Rb, ICOS, or 41BB. In some embodiments, a multispecific binding molecule described herein comprises an Fc region that is mutated to have reduced binding to Fc receptor or reduced ADCC, ADCP, or CDC activity, e.g., an Fc region comprising the mutations D265A, N297A, and P329A (DANAPA), numbered according to the Eu numbering system.

In some embodiments, the first binding domain (e.g., an scFv) is N-terminal of the VH of the second binding domain (e.g., a Fab fragment), e.g., linked via a peptide linker. In some embodiments, the multispecific binding molecule further comprises one or more of (e.g., all of) a CH1, CH2, and CH3, e.g., in order from N-terminal to C-terminal. In some embodiments, a polypeptide of the multispecific binding molecule comprises the following sequences, from N-terminal to C-terminal: VH of the first binding domain, first peptide linker (e.g., a (G4S)4 linker (SEQ ID NO: 29)), VL of first binding domain, second peptide linker (e.g., a (G4S)4 linker (SEQ ID NO: 29)), VH of the second binding domain, CH1, CH2, and CH3. In some embodiments, a polypeptide of the multispecific binding molecule comprises the following sequences: from N-terminal to C-terminal: VL of the second binding domain and CL. In some embodiments, the multispecific binding molecule comprises an Fc region that is mutated to have reduced binding to Fc receptor or reduced ADCC, ADCP, or CDC activity, e.g., an Fc region comprising the mutations D265A, N297A, and P329A (DANAPA), numbered according to the Eu numbering system. In some embodiments, the first binding fragment comprises an anti-CD3 binding domain, e.g., an anti-CD3 scFv, e.g., comprising an anti-CD3 sequence disclosed in Table 19. In some embodiments, the second binding domain comprises a costimulatory molecule binding domain, e.g., an anti-CD2 binding domain, e.g., an anti-CD2 Fab, e.g., comprising an anti-CD2 sequence disclosed in Table 19. In some embodiments, the second binding domain comprises a costimulatory molecule binding domain, e.g., an anti-CD28 binding domain, e.g., an anti-CD28 Fab, e.g., comprising an anti-CD28 sequence disclosed in Table 19. In some embodiments, the first binding fragment comprises a costimulatory molecule binding domain, e.g., an anti-CD2 or anti-CD28 binding domain, e.g., an anti-CD2 or anti-CD28 scFv, e.g., comprising an anti-CD2 or anti-CD28 sequence disclosed in Table 19. In some embodiments, the second binding domain comprises an anti-CD3 binding domain, e.g., an anti-CD3 Fab, e.g., comprising an anti-CD3 sequence disclosed in Table 19. Examples of such multispecific binding molecules are depicted as the top left construct in FIG. 37A; Construct 1 or Construct 2 in FIG. 48A; and Construct 1 or Construct 2 in Table 20.

In some embodiments, the first binding domain (e.g., a Fab fragment) is N-terminal to a second binding domain (e.g., an scFv), e.g., wherein an Fc region is situated between the first and second binding domain. In some embodiments, the Fc region is mutated to have reduced binding to Fc receptor or reduced ADCC, ADCP, or CDC activity, e.g., an Fc region comprising the mutations D265A, N297A, and P329A (DANAPA), numbered according to the Eu numbering system. In some embodiments the Fc region is comprises the mutations L234A, L235A, S267K, and P329A (LALASKPA), numbered according to the Eu numbering system. In some embodiments, the Fc region comprises the mutations L234A, L235A, and P329G (LALAPG), numbered according to the Eu numbering system. In some embodiments, the Fc region comprises the mutations G237A, D265A, P329A, and S267K (GADAPASK), numbered according to the Eu numbering system. In some embodiments, the multispecific binding molecule further comprises one or more of (e.g., all of) a CH1, CH2, and CH3, e.g., in order from N-terminal to C-terminal. In some embodiments, a polypeptide of the multispecific binding molecule comprises the following sequences, from N-terminal to C-terminal: VH of the first binding domain, CH1, CH2, CH3, first peptide linker (e.g., a (G4S)4 linker (SEQ ID NO: 29)), VH of second binding domain, second peptide linker (e.g., a (G4S)4 linker (SEQ ID NO: 29)), and VL of the second binding domain. In some embodiments, a polypeptide of the multispecific binding molecule comprises the following sequences: from N-terminal to C-terminal: VL of the first binding domain and CL. In some embodiments, the first binding domain comprises a costimulatory molecule binding domain, e.g., an anti-CD2 binding domain, e.g., an anti-CD2 Fab, e.g., comprising an anti-CD2 sequence disclosed in Table 19. In some embodiments, the first binding domain comprises a costimulatory molecule binding domain, e.g., an anti-CD28 binding domain, e.g., an anti-CD28 Fab, e.g., comprising an anti-CD28 sequence disclosed in Table 19, e.g., anti-CD28 (1) or anti-CD28 (2). In some embodiments, the second binding domain comprises an anti-CD3 binding domain, e.g., an anti-CD3 scFv, e.g., comprising an anti-CD3 sequence disclosed in Table 19, e.g., anti-CD3 (1), anti-CD3 (2), anti-CD3 (3), or anti-CD3 (4). In some embodiments, the first binding domain comprises an anti-CD3 binding domain, e.g., an anti-CD3 Fab, e.g., comprising an anti-CD3 sequence disclosed in Table 19, e.g., anti-CD3 (1), anti-CD3 (2), anti-CD3 (3), or anti-CD3 (4). In some embodiments, the second binding domain comprises a costimulatory molecule binding domain, e.g., an anti-CD2 or anti-CD28 binding domain, e.g., an anti-CD2 or anti-CD28 scFv, e.g., comprising an anti-CD2 or anti-CD28 sequence disclosed in Table 19. Examples of such multispecific binding molecules are depicted as the second construct from the left in the top row of FIG. 37A; Construct 3 or Construct 4 in FIG. 48A; and Construct 3 or Construct 4 in Table 20. In some embodiments, the first binding domain (e.g., a Fab fragment) is N terminal to a second binding domain (e.g., a scFv), e.g., via a peptide linker. In some embodiments, the multispecific binding molecule further comprises one or more of (e.g., all of) a CH1, CH2, and CH3, e.g., in order from N-terminal to C-terminal. In some embodiments, a polypeptide of the multispecific binding molecule comprises the following sequences, from N-terminal to C-terminal: VH of the first binding domain, CH1, first peptide linker (e.g., a (G4S)2 linker (SEQ ID NO: 767)), VH of the second binding domain, second peptide linker (e.g., a (G4S)4 linker (SEQ ID NO: 29)), VL of the second binding domain, third peptide linker (e.g., a (G4S)4 linker (SEQ ID NO: 29)), CH2, and CH3. In some embodiments, a polypeptide of the multispecific binding molecule comprises the following sequences: from N-terminal to C-terminal: VL of the first binding domain and CL. In some embodiments, the multispecific binding molecule comprises an Fc region that is mutated to have reduced binding to Fc receptor or reduced ADCC, ADCP, or CDC activity, e.g., an Fc region comprising the mutations D265A, N297A, and P329A (DANAPA), numbered according to the Eu numbering system. In some embodiments, the first binding domain comprises a costimulatory molecule binding domain, e.g., an anti-CD2 binding domain, e.g., an anti-CD2 Fab, e.g., comprising an anti-CD2 sequence disclosed in Table 19. In some embodiments, the first binding domain comprises a costimulatory molecule binding domain, e.g., an anti-CD28 binding domain, e.g., an anti-CD28 Fab, e.g., comprising an anti-CD28 sequence disclosed in Table 19, e.g., anti-CD28 (1) or anti-CD28 (2). In some embodiments, the first binding domain comprises a costimulatory molecule binding domain, e.g., an anti-CD25 binding domain (for example, an anti-CD25 Fab), an anti-CD27 binding domain (for example, an anti-CD27 Fab), an anti-IL6Rb binding domain (for example, an anti-TL6Rb Fab), an anti-ICOS binding domain (for example, an anti-ICOS Fab), or an anti-41BB binding domain (for example, an anti-41BB Fab). In some embodiments, the second binding domain comprises an anti-CD3 binding domain, e.g., an anti-CD3 scFv, e.g., comprising an anti-CD3 sequence disclosed in Table 19, e.g., anti-CD3 (1), anti-CD3 (2), anti-CD3 (3), or anti-CD3 (4). In some embodiments, the first binding domain comprises an anti-CD3 binding domain, e.g., an anti-CD3 Fab, e.g., comprising an anti-CD3 sequence disclosed in Table 19, e.g., anti-CD3 (1), anti-CD3 (2), anti-CD3 (3), or anti-CD3 (4). In some embodiments, the second binding domain comprises a costimulatory molecule binding domain, e.g., an anti-CD2 binding domain (for example, an anti-CD2 scFv), an anti-CD28 binding domain (for example, an anti-CD28 scFv), an anti-CD25 binding domain (for example, an anti-CD25 scFv), an anti-CD27 binding domain (for example, an anti-CD27 scFv), an anti-IL6Rb binding domain (for example, an anti-TL6Rb scFv), an anti-ICOS binding domain (for example, an anti-ICOS scFv), or an anti-41BB binding domain (for example, an anti-41BB scFv). Examples of such multispecific binding molecules are depicted as the third construct from the left in the top row of FIG. 37A; Construct 5 or Construct 6 in FIG. 48A; and Construct 5 or Construct 6 in Table 20.

In some embodiments, the first binding domain (e.g., an scFv) is N-terminal to a second binding domain (e.g., a Fab fragment), e.g., wherein an Fc region is situated between the first and second binding domain. In some embodiments, the Fc region is mutated to have reduced binding to Fc receptor or reduced ADCC, ADCP, or CDC activity, e.g., an Fc region comprising the mutations D265A, N297A, and P329A (DANAPA), numbered according to the Eu numbering system. In some embodiments, the multispecific binding molecule further comprises one or more of (e.g., all of) a CH2, CH3, and CH1, e.g., in order from N-terminal to C-terminal. In some embodiments, a polypeptide of the multispecific binding molecule comprises the following sequences, from N-terminal to C-terminal: VH of the first binding domain, first peptide linker (e.g., a (G4S)4 linker) (SEQ ID NO: 29), VL of the first binding domain, second peptide linker (e.g., a (G4S) linker (SEQ ID NO: 768)), CH2, CH3, third peptide linker (e.g., a (G4S)4 linker (SEQ ID NO: 29)), VH of the second binding domain, and CH1. In some embodiments, a polypeptide of the multispecific binding molecule comprises the following sequences: from N-terminal to C-terminal: VL of the second binding domain and CL. In some embodiments, the first binding domain comprises an anti-CD3 binding domain, e.g., an anti-CD3 scFv, e.g., comprising an anti-CD3 sequence disclosed in Table 19. In some embodiments, the second binding domain comprises a costimulatory molecule binding domain, e.g., an anti-CD2 binding domain, e.g., an anti-CD2 Fab, e.g., comprising an anti-CD2 sequence disclosed in Table 19. In some embodiments, the second binding domain comprises a costimulatory molecule binding domain, e.g., an anti-CD28 binding domain, e.g., an anti-CD28 Fab, e.g., comprising an anti-CD28 sequence disclosed in Table 19. In some embodiments, the first binding domain comprises a costimulatory molecule binding domain, e.g., an anti-CD2 or anti-CD28 binding domain, e.g., an anti-CD2 or anti-CD28 scFv, e.g., comprising an anti-CD2 or anti-CD28 sequence disclosed in Table 19. In some embodiments, the second binding domain comprises an anti-CD3 binding domain, e.g., an anti-CD3 Fab, e.g., comprising an anti-CD3 sequence disclosed in Table 19. Examples of such multispecific binding molecules are depicted as the rightmost construct in the top row of FIG. 37A; Construct 7 or Construct 8 in FIG. 48A; and Construct 7 or Construct 8 in Table 20.

In some embodiments, the first binding domain (e.g., a Fab fragment) is situated N terminal to a first Fc region. In some embodiments, the multispecific binding molecule comprises one or more of (e.g., all of) a first CH1, a first CH2, and a first CH3, e.g., in order from N-terminal to C-terminal. In some embodiments, the second binding domain (e.g., an scFv) is situated N terminal to a second Fc region, e.g., in a second polypeptide chain. In some embodiments, the multispecific binding molecule comprises, e.g., in the second polypeptide chain, one or more of (e.g., both of) a second CH2 and a second CH3, e.g., in order from N-terminal to C-terminal. In some embodiments, the multispecific binding molecule comprises a heterodimeric antibody molecule, such as for instance, wherein the first and second Fc regions comprise knob-into-hole mutations. In some embodiments, the first Fc region binds the second Fc region more strongly than the first Fc region binds another copy of the first Fc region. In some embodiments, a first polypeptide of the multispecific binding molecule comprises the following sequences, from N-terminal to C-terminal: VH of the first binding domain, a first CH1, a first CH2, and a first CH3. In some embodiments, a second polypeptide of the multispecific binding molecule comprises the following sequences, from N-terminal to C-terminal: VH of the second binding domain, a first peptide linker (e.g., a (G4S) linker (SEQ ID NO: 768)), VL of the second binding domain, a second CH2, and a second CH3. In some embodiments, a third polypeptide of the multispecific binding molecule comprises the following sequences: from N-terminal to C-terminal: VL of the first binding domain and CL. In some embodiments, the second polypeptide of the multispecific binding molecule further comprises a homomultimerization domain, e.g., a Matrilin1 protein or the coiled-coil domain of cartilage oligomeric matrix protein (COMPcc), C-terminal to the second CH3, e.g., via a peptide linker (e.g., a (G4S)4 linker (SEQ ID NO: 29), a (G4S) linker (SEQ ID NO: 768), or a (G4S)3 linker (SEQ ID NO: 30)). In some embodiments, the multispecific binding molecule comprises two, three, four, or five copies of the first binding domain and the same number of copies of the second binding domain, e.g., as depicted in FIG. 37B. In some embodiments, the first binding domain comprises a costimulatory molecule binding domain, e.g., an anti-CD2 binding domain (for example, an anti-CD2 Fab). In some embodiments, the first binding domain comprises a costimulatory molecule binding domain, e.g., an anti-CD28 binding domain (for example, an anti-CD28 Fab). In some embodiments, the second binding domain comprises an anti-CD3 binding domain, e.g., an anti-CD3 scFv. Examples of such multispecific binding molecules are depicted as the leftmost construct in the bottom row of FIG. 37A; constructs in FIG. 37B, Construct 9, Construct 10, Construct 12, Construct 13, Construct 15, and Construct 16 in FIG. 48B; and Construct 9, Construct 10, Construct 12, Construct 13, Construct 15, and Construct 16 in Table 20.

In some embodiments, a binding molecule described herein comprises a binding domain. In some embodiments the binding domain (e.g., an scFv) is situated N terminal to an Fc region. In some embodiments, the binding molecule comprises a heterodimeric antibody molecule, such as for instance, wherein the first and second Fc regions comprise knob-into-hole mutations. In some embodiments, the first Fc region binds the second Fc region more strongly than the first Fc region binds another copy of the first Fc region. In some embodiments, the binding molecule comprises one or more of (e.g., all of) a CH2 and a CH3, e.g., in order from N-terminal to C-terminal. In some embodiments, a second polypeptide of the binding molecule comprises the following sequences, from N-terminal to C-terminal: VH of the binding domain, first peptide linker (e.g., a (G4S)4 linker (SEQ ID NO: 29)), VL of the binding domain, second peptide linker (e.g., (G4S)4 linker (SEQ ID NO: 29) or (G4S) linker (SEQ ID NO: 768)), CH2, and CH3. In some embodiments, the second polypeptide of the binding molecule further comprises a homomultimerization domain, e.g., a Matrilin1 protein or the coiled-coil domain of cartilage oligomeric matrix protein (COMPcc), C-terminal to the second CH3, e.g., via a peptide linker (e.g., (G4S)4 linker (SEQ ID NO: 29), (GS4)3 linker (SEQ ID NO: 878), or (G4S) linker (SEQ ID NO: 768)). In some embodiments, the binding molecule comprises two, three, four, or five copies of the binding, e.g., as depicted in FIG. 37B. In some embodiments, the binding domain comprises an anti-CD3 binding domain, e.g., an anti-CD3 scFv. In some embodiments, a costimulatory molecule binding domain is absent. Examples of such binding molecules are depicted as the rightmost construct in the bottom row of FIG. 37A; Construct 11, Construct 14, and Construct 17 in FIG. 48B; and Construct 11, Construct 14, and Construct 17 in Table 20.

In some embodiments, the multispecific binding protein comprises an anti-CD28 binding domain, e.g., an anti-CD28 Fab, e.g., comprising an anti-CD28 (2) sequence of Table 19 (or a sequence having at least 70%, 75%, 80%, 85%, 90%, 95%, 96%, 97%, 98%, or 99% sequence identity thereto), and an anti-CD3 scFv, e.g., comprising an anti-CD3 (4) sequence of Table 19 (or a sequence having at least 70%, 75%, 80%, 85%, 90%, 95%, 96%, 97%, 98%, or 99% sequence identity thereto). In some embodiments, the multispecific binding protein comprises an Fc region, wherein the anti-CD28 Fab is fused to the Fc region, which is further fused to the anti-CD3 scFv. In some embodiments, the Fc region comprises L234A, L235A, S267K, and P329A mutations (LALASKPA), numbered according to the Eu numbering system. In some embodiments, the multispecific binding protein comprises a heavy chain comprising the amino acid sequence of SEQ ID NO: 726 or 1416, or an amino acid sequence at least 70%, 75%, 80%, 85%, 90%, 95%, 96%, 97%, 98%, or 99% identical to SEQ ID NO: 726 or 1416. In some embodiments, the multispecific binding protein comprises a light chain comprising the amino acid sequence of SEQ ID NO: 728 or 730, or an amino acid sequence at least 70%, 75%, 80%, 85%, 90%, 95%, 96%, 97%, 98%, or 99% identical to SEQ ID NO: 728 or 730. In some embodiments, the multispecific binding protein comprises a heavy chain comprising the amino acid sequence of SEQ ID NO: 726 or 1416, or an amino acid sequence at least 70%, 75%, 80%, 85%, 90%, 95%, 96%, 97%, 98%, or 99% identical to SEQ ID NO: 726 or 1416, and a light chain comprising the amino acid sequence of SEQ ID NO: 728 or 730, or an amino acid sequence at least 70%, 75%, 80%, 85%, 90%, 95%, 96%, 97%, 98%, or 99% identical to SEQ ID NO: 728 or 730. In some embodiments, the multispecific binding protein comprises a heavy chain comprising the amino acid sequence of SEQ ID NO: 726, or an amino acid sequence at least 70%, 75%, 80%, 85%, 90%, 95%, 96%, 97%, 98%, or 99% identical thereto, and a light chain comprising the amino acid sequence of SEQ ID NO: 728, or an amino acid sequence at least 70%, 75%, 80%, 85%, 90%, 95%, 96%, 97%, 98%, or 99% identical thereto. In some embodiments, the multispecific binding protein comprises a heavy chain comprising the amino acid sequence of SEQ ID NO: 726, or an amino acid sequence at least 70%, 75%, 80%, 85%, 90%, 95%, 96%, 97%, 98%, or 99% identical thereto, and a light chain comprising the amino acid sequence of SEQ ID NO: 730, or an amino acid sequence at least 70%, 75%, 80%, 85%, 90%, 95%, 96%, 97%, 98%, or 99% identical thereto. In some embodiments, the multispecific binding protein comprises a heavy chain comprising the amino acid sequence of SEQ ID NO: 1416, or an amino acid sequence at least 70%, 75%, 80%, 85%, 90%, 95%, 96%, 97%, 98%, or 99% identical thereto, and a light chain comprising the amino acid sequence of SEQ ID NO: 728, or an amino acid sequence at least 70%, 75%, 80%, 85%, 90%, 95%, 96%, 97%, 98%, or 99% identical thereto. In some embodiments, the multispecific binding protein comprises a heavy chain comprising the amino acid sequence of SEQ ID NO: 1416, or an amino acid sequence at least 70%, 75%, 80%, 85%, 90%, 95%, 96%, 97%, 98%, or 99% identical thereto, and a light chain comprising the amino acid sequence of SEQ ID NO: 730, or an amino acid sequence at least 70%, 75%, 80%, 85%, 90%, 95%, 96%, 97%, 98%, or 99% identical thereto.

In some embodiments, the multispecific binding protein comprises an anti-CD28 binding domain, e.g., an anti-CD28 Fab, e.g., comprising an anti-CD28 (2) sequence of Table 19 (or a sequence having at least 70%, 75%, 80%, 85%, 90%, 95%, 96%, 97%, 98%, or 99% sequence identity thereto), and an anti-CD3 scFv, e.g., comprising an anti-CD3 (2) sequence of Table 19 (or a sequence having at least 70%, 75%, 80%, 85%, 90%, 95%, 96%, 97%, 98%, or 99% sequence identity thereto). In some embodiments, the multispecific binding protein comprises an Fc region, wherein the anti-CD28 Fab is fused to the Fc region, which is further fused to the anti-CD3 scFv. In some embodiments, the Fc region comprises L234A, L235A, S267K, and P329A mutations (LALASKPA), numbered according to the Eu numbering system, numbered according to the Eu numbering system. In some embodiments, the multispecific binding protein comprises a heavy chain comprising the amino acid sequence of SEQ ID NO: 893 or 1417, or an amino acid sequence at least 70%, 75%, 80%, 85%, 90%, 95%, 96%, 97%, 98%, or 99% identical to SEQ ID NO: 893 or 1417. In some embodiments, the multispecific binding protein comprises a light chain comprising the amino acid sequence of SEQ ID NO: 892, or an amino acid sequence at least 70%, 75%, 80%, 85%, 90%, 95%, 96%, 97%, 98%, or 99% identical thereto. In some embodiments, the multispecific binding protein comprises a heavy chain comprising the amino acid sequence of SEQ ID NO: 893, or an amino acid sequence at least 70%, 75%, 80%, 85%, 90%, 95%, 96%, 97%, 98%, or 99% identical thereto, and a light chain comprising the amino acid sequence of SEQ ID NO: 892, or an amino acid sequence at least 70%, 75%, 80%, 85%, 90%, 95%, 96%, 97%, 98%, or 99% identical thereto. In some embodiments, the multispecific binding protein comprises a heavy chain comprising the amino acid sequence of SEQ ID NO: 1417, or an amino acid sequence at least 70%, 75%, 80%, 85%, 90%, 95%, 96%, 97%, 98%, or 99% identical thereto, and a light chain comprising the amino acid sequence of SEQ ID NO: 892, or an amino acid sequence at least 70%, 75%, 80%, 85%, 90%, 95%, 96%, 97%, 98%, or 99% identical thereto.

In some embodiments, the multispecific binding protein comprises an anti-CD28 binding domain, e.g., an anti-CD28 Fab, e.g., comprising an anti-CD28 (1) sequence of Table 19 (or a sequence having at least 70%, 75%, 80%, 85%, 90%, 95%, 96%, 97%, 98%, or 99% sequence identity thereto) and an anti-CD3 scFv, e.g., comprising an anti-CD3 (4) sequence of Table 19 (or a sequence having at least 70%, 75%, 80%, 85%, 90%, 95%, 96%, 97%, 98%, or 99% sequence identity thereto). In some embodiments, the multispecific binding protein comprises an Fc region, wherein the anti-CD28 Fab is fused to the Fc region, which is further fused to the anti-CD3 scFv. In some embodiments, the Fc region comprises L234A, L235A, S267K, and P329A mutations (LALASKPA), numbered according to the Eu numbering system. In some embodiments, the multispecific binding protein comprises a heavy chain comprising the amino acid sequence of SEQ ID NO: 895, or an amino acid sequence at least 70%, 75%, 80%, 85%, 90%, 95%, 96%, 97%, 98%, or 99% identical thereto. In some embodiments, the multispecific binding protein comprises a light chain comprising the amino acid sequence of SEQ ID NO: 894, or an amino acid sequence at least 70%, 75%, 80%, 85%, 90%, 95%, 96%, 97%, 98%, or 99% identical thereto. In some embodiments, the multispecific binding protein comprises a heavy chain comprising the amino acid sequence of SEQ ID NO: 895, or an amino acid sequence at least 70%, 75%, 80%, 85%, 90%, 95%, 96%, 97%, 98%, or 99% identical thereto, and a light chain comprising the amino acid sequence of SEQ ID NO: 894, or an amino acid sequence at least 70%, 75%, 80%, 85%, 90%, 95%, 96%, 97%, 98%, or 99% identical thereto.

It is understood that in many of the embodiments herein, a multispecific binding molecule comprises two or more polypeptide chains that are covalently linked to each other, e.g., via a disulfide bridge. However, in some embodiments, the two or more polypeptide chains of the multispecific binding molecule may be noncovalently bound to each other.

It is also understood that a Fab fragment may be present as part of a larger protein, for instance, a Fab fragment may be fused with CH2 and CH3 and thus be part of full length antibody.

The multispecific binding molecule comprising an agent that stimulates a CD3/TCR complex and an agent that stimulates a costimulatory molecule and/or growth factor receptor disclosed herein is contemplated for use as a cell activation agent disclosed herein.

### Fc Variants

In some embodiments, a multispecific binding molecule described herein comprises an Fc region, e.g., as described herein. In some embodiments, the Fc region is a wild type Fc region, e.g., a wild type human Fc region. In some embodiments, the Fc region comprises a variant, e.g., an Fc region comprising an addition, substitution, or deletion of at least one amino acid residue in the Fc region which results in, e.g., reduced or ablated affinity for at least one Fc receptor. In some embodiments, the multispecific binding molecule comprises the amino acid sequence of an Fc region provided in Table 20, or a sequence having at least 70%, 75%, 80%, 85%, 90%, 95%, 96%, 97%, 98%, or 99% sequence identity thereto.

In some embodiments, the Fc region of an antibody interacts with a number of receptors or ligands including Fc Receptors (e.g., FcγRI, FcγRIIA, FcγRIIIA), the complement protein CIq, and other molecules such as proteins A and G. These interactions promote a variety of effector functions and downstream signaling events including: antibody dependent cell-mediated cytotoxicity (ADCC), Antibody-dependent cellular phagocytosis (ADCP) and complement dependent cytotoxicity (CDC). Non-limiting exemplary Fc regions featuring these and other silencing modifications disclosed herein are provided in Table 20 above.

In some embodiments, a multispecific binding molecule described herein comprising a variant Fc region has reduced, e.g., ablated, affinity for an Fc receptor, e.g., an Fc receptor described herein. In some embodiments, the reduced affinity is compared to an otherwise similar antibody with a wild type Fc region.

In some embodiments, a multispecific binding molecule described herein comprising a variant Fc region has one or more of the following properties: (1) reduced effector function (e.g., reduced ADCC, ADCP and/or CDC); (2) reduced binding to one or more Fc receptors; and/or (3) reduced binding to C1q complement. In some embodiments, the reduction in any one, or all of properties (1)-(3) is compared to an otherwise similar antibody with a wildtype Fc region. Exemplary Fc region variants are provided in Table 34 and also disclosed in Saunders 0, (2019) Frontiers in Immunology; vol 10, article1296, the entire contents of which is hereby incorporated by reference.

In some embodiments, a multispecific binding molecule described herein comprises any one or all, or any combination of Fc region variants, e.g., mutations, disclosed in Table 34. In some embodiments, the Fc region of a multispecific binding molecule described herein is silenced. In some embodiments, the Fc region of a multispecific binding protein described herein is silenced by a combination of amino acid substitutions selected from the group consisting of LALA, DAPA, DANAPA, LALADANAPS, LALAGA, LALASKPA, DAPASK, GADAPA, GADAPASK, LALAPG, and LALAPA (numbered according to the Eu numbering system).

In some embodiments, a multispecific binding molecule described herein comprises any one or all, or any combination of a mutant comprising a L234, e.g., L234A and/or L235, e.g., L234A mutation (LALA) in the IgG1 Fc amino acid sequence, numbered according to the Eu numbering system; D265, e.g., D265A and/or P329, e.g., P329A (DAPA), numbered according to the Eu numbering system; N297, e.g., N297A, numbered according to the Eu numbering system; DANAPA (D265A, N297A, and P329A), numbered according to the Eu numbering system; and/or L234, e.g. L234A, L235, e.g., L235A, D265, e.g., D265A, N297, e.g., N297A, and P331, e.g., P331S (LALADANAPS), numbered according to the Eu numbering system. In some embodiments, a multispecific binding molecule described herein comprises a human IgG1 Fc variant of a wild-type human IgG1 Fc region, wherein the Fc variant comprises any one or all of: an L234 (e.g., L234A), L235 (e.g., L235A), and/or G237 (e.g., G237A) mutation (LALAGA), numbered according to the Eu numbering system; an L234 (e.g., L234A), L235 (e.g., L235A), S267 (e.g., S267K), and/or P329 (e.g., P329A) mutation (LALASKPA), numbered according to the Eu numbering system; a D265 (e.g., D265A), P329 (e.g., P329A), and/or S267 (e.g., S267K) mutation (DAPASK), numbered according to the Eu numbering system; a G237 (e.g., G237A), D265 (e.g., D265A), and/or P329 (e.g., P329A) mutation (GADAPA), numbered according to the Eu numbering system; a G237 (e.g., G237A), D265 (e.g., D265A), P329 (e.g., P329A), and/or S267 (e.g., S267K) mutation (GADAPASK), numbered according to the Eu numbering system; a L234 (e.g., L234A), L235 (e.g., L235A), and/or P329 (e.g., P329G) mutation (LALAPG), numbered according to the Eu numbering system; or a L234 (e.g., L234A), L235 (e.g., L235A), and/or P329 (e.g., P329A) mutation (LALAPA), wherein the amino acid residues are numbered according to the Eu numbering system.

In some embodiments, the Fc region of a multispecific binding protein described herein comprises a mutation that results in reduced binding to an Fc receptor or reduced ADCC, ADCP, or CDC activity, e.g., an Fc region comprising: a D265 (e.g., D265A), N297 (e.g., N297A), and P329 (e.g., P329A) mutation (DANAPA), numbered according to the Eu numbering system; an L234 (e.g., L234A), L235 (e.g., L235A), and G237 (G237A) mutation (LALAGA), numbered according to the Eu numbering system; an L234 (L234A), L235 (e.g., L235A), S267 (e.g., S267K), and P329 (e.g., P329A) mutation (LALASKPA), numbered according to the Eu numbering system; a D265 (e.g., D265A), P329 (e.g., P329A), and S267 (e.g., S267K) mutation (DAPASK), numbered according to the Eu numbering system; a G237 (e.g., G237A), a D265 (e.g., D265A), and P329 (P329A) mutation (GADAPA), numbered according to the Eu numbering system; a G237 (e.g., G237A), D265 (e.g., D265A), P329 (e.g., P329A), and S267 (e.g., S267K) mutation (GADAPASK), numbered according to the Eu numbering system; an L234 (e.g., L234A), L235 (e.g., L235A), and P329 (e.g., P329G) mutation (LALAPG), numbered according to the Eu numbering system; or an L234 (e.g., L234A), L235 (e.g., L235A), and P329 (e.g., P329A) mutation (LALAPA), numbered according to the Eu numbering system.

It is understood that the terms “LALA,” “DAPA,” “DANAPA,” “LALADANAPS,” “LALAGA”, “LALASKPA”, “DAPASK”, “GADAPA”, “GADAPASK”, “LALAPG”, and “LALAPA” represent shorthand terminology for the different combinations of substitutions described herein rather than contiguous amino acid sequences.

TABLE 34
Exemplary Fc modifications

| Modification or mutation | Altered effector function |
|---|---|
| Leu235Glu | ADCC; |
| Leu234Ala/Leu235Ala (LALA) | ADCC; ADCP; CDC |
| Ser228Pro/Leu235Glu | |
| Leu234Ala/Leu235Ala/Pro329Gly | ADCP |
| Pro331Ser/Leu234Glu/Leu235Phe | CDC |
| Asp265Ala | ADCC, ADCP |
| Gly237Ala | ADCP |
| Glu318Ala | ADCP |
| Glu233Pro | |
| Gly236Arg/Leu328Arg | ADCC |
| His268Gln/Val309Leu/Ala330Ser/Pro331Ser | ADCC; ADCP; CDC |
| Val234Ala/Gly237Ala/Pro238Ser/ | ADCC; ADCP; CDC |
| His268Ala/Val309Leu/Ala330Ser/Pro331Ser | |
| Leu234Ala/L235Ala/Gly237Ala/P238Ser/ | ADCC; CDC |
| His268Ala/Ala330Ser/Pro331Ser | |
| Ala330Leu | CDC |
| Asp270Ala | CDC |
| Lys322Ala | CDC |
| Pro329Ala | CDC |
| Pro331Ala | CDC |
| Val264Ala | CDC |
| High mannose glycosylation | CDC |
| Phe241Ala | CDC |
| Asn297Ala or Gly or Gln | ADCC; ADCP; CDC |
| S228P/Phe234Ala/Leu235Ala | ADCC; CDC |

Compositions of Mesoporous Silica Particles, Viral Vectors, and Cell Activation Agents

Also described herein is a composition comprising an extended release agent, e.g., a population of mesoporous silica particles, and a viral vector. Also described herein is a composition, comprising a first population of mesoporous silica particles and a viral vector. In some embodiments, the MSPs (e.g., MSRs) further comprise a plurality of functional groups adsorbed or covalently bonded onto the surfaces lining the pores and/or nanochannels or the surface. In some embodiments, the functional group is a —OH (hydroxyl), amine, carboxylic acid, phosphonate, halide, azide, alkyne, epoxide, sulfhydryl, disulfide, polyethyleneimine, hydrophobic moiety or salts thereof In some embodiments, the functional group (i.e. —OH (hydroxyl), amine, carboxylic acid, phosphonate, halide, azide, alkyne, epoxide, sulfhydryl, disulfide, polyethyleneimine, hydrophobic moiety, or salts thereof) may be directly attached to the surface of the MSP. In some embodiments, the functional group is covalently bonded to the MSP (e.g., MSR) surface via a C₁ to C₂₀ alkyl linker. In other embodiments, the functional group is covalently bonded to the MSP surface via a polyethyleneglycol linker. In particular embodiments, the polyethylene glycol linker has the formula (—O(CH2-CH₂—)₁₋₂₅. In particular embodiments, the surface modification is a C₁ to C₂₀ alkyl perhaloalkyl or a C₁ to C₂₀ alkyl perfluoroalkyl.

In some embodiments, the MSPs (e.g., MSRs) are surface modified with a primary, secondary, tertiary, or quaternary amine. In particular embodiments, the mesoporous silica rods are modified with polyethyleneimine. In specific embodiments, the polyethyleneimine is branched or unbranched. In alternate embodiments, the polyethyleneimine group has an average molecular weight in the range of about 1000 to 20,000 Daltons (Da), as measured by gel permeation chromatography (GPC). In some embodiments, the polyethyleneimine group has an average molecular weight of about 1,200 to 15,000 Da, about 1,500 to 12,000 Da, about 2,000 Da, about 3,000 Da, about 4,000 Da, about 5,000 Da, about 6,000 Da, about 7,000 Da, about 8,000 Da, about 9,000 Da, or about 10,000 Da, as measured by gel permeation chromatography (GPC).

In some embodiments, the viral vector is conjugated to the mesoporous silica particles. In some embodiments, the viral vector is electrostatically or covalently conjugated to the mesoporous silica particles. In some embodiments, the electrostatic conjugation between the mesoporous silica particles and the viral vector is due to oppositely surface-charged viral vectors and mesoporous silica particles. For example, and without being bound by theory, mesoporous silica particles that are surface modified by polyethyleneimine or primary, secondary, tertiary, or quaternary ammonium groups that are positively charged can be conjugated to negatively surface-charged viral vectors. Thus, in some embodiments, the viral vector is negatively charged, and the mesoporous silica particles are positively charged. In some embodiments, the covalent conjugation between the mesoporous silica particles and the viral vector is achieved by methods known to those of skill in the art, either via linkers or without linkers. For example, and without limitation, the linkers may be polyethylene glycol, alkyl groups, polymers, polyamide linkages, etc.

In some aspects, provided herein includes pharmaceutical compositions comprising mesoporous silica particles as described herein, formulated for use in the manufacture of a population of immune effector cells, e.g., T lymphocyte cells. In some embodiments, the T lymphocyte cells are transduced with a CAR. In some embodiments, the MSPs are conjugated to a viral vector as described herein. In some embodiments, the MSPs are conjugated to a cell activation agent. IN some embodiments a cell activation agent is absorbed on the MSPs. In some embodiments, the MSPs for use in in the manufacture of a population of immune effector cells, e.g., T lymphocyte cells, may be surface modified as described herein.

In some embodiments, the composition is suitable for use as an injectable composition comprising mesoporous silica particles, a viral vector, and, optionally, a cell activation agent, wherein the viral vector comprises an expression vector comprising a recombinant polynucleotide comprising an expression control sequence operatively linked to a nucleotide sequence to be expressed. In some embodiments, the viral vector is conjugated to the mesoporous silica particles as described herein. In some embodiments, the cell activation agent is absorbed on or conjugated to the mesoporous silica particles as described herein. Adsorption to the MSP (e.g., MSR) surface is as commonly understood as a molecule adhering to the surface.

Without wishing to be bound by theory, in some embodiments, an MSP-virus composition, e.g., a composition comprising an MSP and a viral vector, e.g., a viral vector encoding a CAR, limits viral vector drainage to the draining lymph node, decreasing potential off-site transduction, compared to an otherwise similar composition lacking an MSP.

In compositions of mesoporous silica particles described herein, the MSPs (e.g., MSRs) may be present in a concentration of 0.01 to 1000 μg/ml. In alternative embodiments, the concentration of MSPs or MSRs in the compositions described herein may be 0.1 to 500 μg/ml, 0.5 to 100 μg/ml, 1 to 90 μg/ml, 1 to 80 μg/ml, 1 to 70 μg/ml, 1 to 60 μg/ml, 1 to 50 μg/ml, or 1 to 40 μg/ml.

In particular embodiments, the MSPs (e.g., MSRs) may be present in a concentration of about 1 μg/ml, 10 μg/ml, 20 μg/ml, 30 μg/ml, 40 μg/ml, 50 μg/ml, 60 μg/ml, 70 μg/ml, 80 μg/ml, 90 μg/ml, 100 μg/ml, 110 μg/ml, 120 μg/ml, 130 μg/ml, 140 μg/ml, or 150 μg/ml.

In general, compositions described herein may be administered in therapeutically effective amounts as described above via any of the usual and acceptable modes known in the art, either singly or in combination with one or more therapeutic agents.

Injectable compositions may be aqueous isotonic suspensions. The compositions may be sterilized and/or contain adjuvants, such as preserving, stabilizing, wetting or emulsifying agents, solution promoters, salts for regulating the osmotic pressure and/or buffers. In addition, they may also contain other therapeutically effective substances.

Pharmaceutical compositions of the present invention may be administered in a manner appropriate to the disease to be treated (or prevented). The quantity and frequency of administration will be determined by such factors as the condition of the patient, and the type and severity of the patient's disease, although appropriate dosages may be determined by clinical trials.

In some embodiments, the pharmaceutical composition is substantially free of, e.g., there are no detectable levels of a contaminant, e.g., selected from the group consisting of endotoxin, mycoplasma, replication competent lentivirus (RCL), p24, VSV-G nucleic acid, HIV gag, residual anti-CD3/anti-CD28 coated beads, mouse antibodies, pooled human serum, bovine serum albumin, bovine serum, culture media components, vector packaging cell or plasmid components, a bacterium and a fungus. In some embodiments, the bacterium is at least one selected from the group consisting of Alcaligenes faecalis, Candida albicans, Escherichia coli, Haemophilus influenza, Neisseria meningitides, Pseudomonas aeruginosa, Staphylococcus aureus, Streptococcus pneumonia, and Streptococcus pyogenes group A.

The pharmaceutical composition (or formulation) for application may be packaged in a variety of ways depending upon the method used for administering the compositions described herein. Generally, an article for distribution includes a container having deposited therein the pharmaceutical formulation in an appropriate form. Suitable containers are well-known to those skilled in the art and include materials such as bottles (plastic and glass), sachets, ampoules, plastic bags, metal cylinders, and the like. The container may also include a tamper-proof assemblage to prevent indiscreet access to the contents of the package. In addition, the container has deposited thereon a label that describes the contents of the container. The label may also include appropriate warnings.

In some embodiments, the compositions described herein further include a cell activation agent. In some embodiments, cell activation agent is a T cell stimulating compound, an anti-idiotype antibody to a CAR antigen binding domain, and/or tumor antigen. In some embodiments, the cell activation agent is conjugated to or adsorbed on the first population of mesoporous silica particles. In additional or alternative embodiments, the T cell stimulating compound or tumor antigen is conjugated to or adsorbed on to a second population of mesoporous silica particles. In further embodiments, the T-cell stimulating compound or tumor antigen is IL-2, IL-15, GM-CSF, anti-CD2 mAb, anti-CD3 mAb, anti-CD28 mAb, neo-antigen peptides, peptides from shared antigens such as TRP2, gp100, tumor cell lysate, CD19, CD20, CD22, ROR1, mesothelin, CD33/IL3Ra, c-Met, PSMA, Glycolipid F77, EGFRvIII, GD-2, NY-ESO-1 TCR, and/or MAGE A3 TCR. In some embodiments, the cell activation agent comprises a CD3/TCR complex and/or an agent that stimulates a costimulatory molecule and/or growth factor receptor, optionally, wherein the cell activation agent is a multispecific binding molecule comprising an agent that stimulates a CD3/TCR complex and an agent that stimulates a costimulatory molecule and/or growth factor receptor

In embodiments where the cell activation agent is conjugated to the second population of mesoporous silica particles, the T cell stimulating compound or tumor antigen may be conjugated to a lipid bilayer on the surface of the second population of mesoporous silica particles. Methods of making lipid bilayers on the mesoporous silica particles are known. See e.g., International Appl. Publ. No. WO 2018/013797. Briefly, liposomes containing predefined amounts of a label such as biotin are used to coat the MSPs. The labels may then be used to affix to the T-cell stimulating compound using a complementary label, e.g., streptavidin. Lipids used to make liposomes are known to those of skill in the art and include, without limitation, vesicle-forming lipids having two hydrocarbon chains, typically acyl chains, and a polar head group. Included in this class are the phospholipids, such as phosphatidylcholine (PC), phosphatidylethanolamine (PE), phosphatidic acid (PA), phosphatidylinositol (PI), and sphingomyelin (SM), where the two hydrocarbon chains are typically between about 14-22 carbon atoms in length and have varying degrees of unsaturation. In some embodiments, the lipid is a relatively unsaturated phospholipid (having one, two or three double bonds in the hydrocarbon chain). In some embodiments, the lipid is a phosphatidylcholine. Phosphatidylcholine is a phospholipid that incorporates choline as a headgroup and combines a glycerophosphoric acid with two fatty acids. In some embodiments, the phosphatidylcholine is a palmitoyl phosphatidylcholine or a oleoyl phosphatidylcholine or a 1-palmitoyl,2-oleoyl-phosphatidylcholine. More than one type of lipid may be used in preparing the liposome composition. The selection of lipids and proportions can be varied to achieve a desired degree of fluidity or rigidity, and/or to control stability. Where more than one type of lipid is used in preparing the liposome composition, a suitable amount of the relatively unsaturated lipid (such as PC) should be used in order to form stable liposomes. In some embodiments, at least 45-50 mol % of the lipids used in the formulation are PC. The liposomes may also include lipids derivatized with a hydrophilic polymer such as polyethylene glycol (PEG). Suitable hydrophilic polymers include polyvinylpyrrolidone, polyvinylmethylether, polymethyloxazoline, polyethyloxazoline, polyhydroxypropyloxazoline, polyhydroxypropylmethacrylamide, polymethacrylamide, polydimethylacrylamide, polyhydroxypropylmethacrylate, polyhydroxyethylacrylate, hydroxymethylcellulose, hydroxyethylcellulose, polyethyleneglycol, polyaspartamide, and hydrophilic peptide sequences. Methods of preparing lipids derivatized with hydrophilic polymers are known (see e.g. U.S. Pat. No. 5,395,619, which is incorporated herein by reference).

In some embodiments, the first population or second population of mesoporous silica particles further includes a cytokine. The cytokine may be, without limitation, IL-1, IL-2, IL-4, IL-5, IL-7, IL-10, IL-12, IL-15, IL-17, IL-21, or transforming growth factor beta (TGF-β), or an agonist thereof, a mimetic thereof, a variant thereof, a functional fragment thereof, or a combination thereof. In particular embodiments, the cytokine is conjugated to or adsorbed on the first or second population of mesoporous silica particles. In embodiments, where the cytokine is adsorbed on the second population of mesoporous silica particles, the second population of MSPs (e.g., MSRs) may be further covered by a lipid bilayer, as described above.

Methods

Aspects disclosed herein relate to a method of transducing cells in vivo comprising administering a biomaterial comprising a cell recruitment factor; an extended release agent, e.g., a first population of mesoporous silica particles; a viral vector; and, optionally, a cell activation agent to a subject. In some embodiments, the components are administered simultaneously or sequentially. In some embodiments, the biomaterial comprising the cell recruitment factor is administered first. In some embodiments, the first population of mesoporous silica rods, the viral vector, and, optionally, the cell activation agent is administered simultaneously and, optionally, after the biomaterial.

Aspects disclosed herein relate to a method of transducing cells in vivo comprising administering a biomaterial and a cell recruitment factor; an extended release agent, e.g., a first population of mesoporous silica particles; a viral vector; and, optionally, a cell activation agent to a subject. In some embodiments, the components are administered simultaneously or sequentially. In some embodiments, the biomaterial and the cell recruitment factor are administered first. In some embodiments, the first population of mesoporous silica rods, the viral vector, and, optionally, the cell activation agent is administered simultaneously and, optionally, after the biomaterial and the cell recruitment factor.

In some embodiments, the method further comprises: contacting T lymphocytes with a composition comprising a first population of mesoporous silica particles (e.g., MSRs), a viral vector, and, optionally, a cell activation agent; wherein the viral vector comprises an expression vector comprising the recombinant polynucleotide comprising an expression control sequence operatively linked to a nucleotide sequence to be expressed.

In some embodiments of the presently described methods, the methods result in an increase in the proportion of T lymphocytes in the population. In some embodiments, method comprises delivering a viral vector to a desired site of action in a subject.

Further aspects relate to a method of treating a subject having a disease, disorder, or condition comprising administering a biomaterial comprising a cell recruitment factor; an extended release agent, e.g., a first population of mesoporous silica particles; a viral vector; and, optionally, a cell activation agent to a subject. In some embodiments, the components are administered simultaneously or sequentially. In some embodiments, the biomaterial comprising the cell recruitment factor is administered first. In some embodiments, the first population of mesoporous silica rods, the viral vector, and, optionally, the cell activation agent is administered simultaneously and, optionally, after the biomaterial.

Further aspects relate to a method of treating a subject having a disease, disorder, or condition comprising administering a biomaterial and a cell recruitment factor; an extended release agent, e.g., a first population of mesoporous silica particles; a viral vector; and, optionally, a cell activation agent to a subject. In some embodiments, the components are administered simultaneously or sequentially. In some embodiments, the biomaterial and the cell recruitment factor are administered first. In some embodiments, the first population of mesoporous silica rods, the viral vector, and, optionally, the cell activation agent is administered simultaneously and, optionally, after the biomaterial and the cell recruitment factor.

In some embodiments, the subject has cancer. In some embodiments, the subject has cancer expressing one or more tumor antigen selected from the group consisting of: TSHR, CD19, CD123, CD22, CD30, CD171, CS-1, CLL-1, CD33, EGFRvIII, GD2, GD3, BCMA, Tn Ag, PSMA, ROR1, FLT3, FAP, TAG72, CD38, CD44v6, CEA, EPCAM, B7H3, KIT, IL-13Ra2, Mesothelin, IL-11Ra, PSCA, PRSS21, VEGFR2, LewisY, CD24, PDGFR-beta, SSEA-4, CD20, Folate receptor alpha, ERBB2 (Her2/neu), MUC1, EGFR, NCAM, Prostase, PAP, ELF2M, Ephrin B2, IGF-I receptor, CAIX, LMP2, gp100, bcr-abl, tyrosinase, EphA2, Fucosyl GM1, sLe, GM3, TGS5, HMWMAA, o-acetyl-GD2, Folate receptor beta, TEM1/CD248, TEM7R, CLDN6, GPRC5D, CXORF61, CD97, CD179a, ALK, Polysialic acid, PLAC1, GloboH, NY-BR-1, UPK2, HAVCR1, ADRB3, PANX3, GPR20, LY6K, OR51E2, TARP, WT1, NY-ESO-1, LAGE-1a, MAGE-A1, legumain, HPV E6,E7, MAGE A1, ETV6-AML, sperm protein 17, XAGE1, Tie 2, MAD-CT-1, MAD-CT-2, Fos-related antigen 1, p53, p53 mutant, prostein, survivin and telomerase, PCTA-1/Galectin 8, MelanA/MART1, Ras mutant, hTERT, sarcoma translocation breakpoints, ML-IAP, ERG (TMPRSS2 ETS fusion gene), NA17, PAX3, Androgen receptor, Cyclin B1, MYCN, RhoC, TRP-2, CYP1B1, BORIS, SART3, PAX5, OY-TES1, LCK, AKAP-4, SSX2, RAGE-1, human telomerase reverse transcriptase, RU1, RU2, intestinal carboxyl esterase, mut hsp70-2, CD79a, CD79b, CD72, LAIR1, FCAR, LILRA2, CD300LF, CLEC12A, BST2, EMR2, LY75, GPC3, FCRL5, IGLL1, and any combination thereof.

In some embodiments, the method further comprises: administering to a subject a composition comprising a first population of mesoporous silica particles and a viral vector; wherein the viral vector comprises an expression vector comprising a recombinant polynucleotide comprising an expression control sequence operatively linked to a nucleotide sequence that expresses a chimeric antigen receptor (CAR) that is engineered to target the tumor antigen.

In some embodiments of the method, the composition further comprises a T cell stimulating compound or tumor antigen conjugated to or adsorbed on the first population of mesoporous silica particles or a second population of mesoporous silica particles, or both populations of MSPs (e.g., MSRs). Alternatively, the method includes administering a second extended release agent, e.g., a second population of mesoporous silica particles in combination with, e.g., simultaneously or shortly after, administration of the first population of MSPs (e.g., MSRs). Alternatively, the second population of MSPs (e.g., MSRs) may be administered after a prolonged period of time after administration of the first population of MSPs.

In some embodiments, the extended release agent comprises a first population of MSPs and the second extended release agent comprises a second population of MSPs.

In some embodiments, the method comprises administering a cell activation agent, wherein the cell activation agent is conjugated to or adsorbed on the first or second population of mesoporous silica particles.

In some embodiments, the second population of MSPs (e.g., MSRs) is administered to the subject simultaneously (e.g., administered on the same day) with or shortly after administration (e.g., administered 1 day, 2 days, 3 days, 4 days, 5 days, 6 days, or 7 days after administration) of the first population of MSPs. In other embodiments, the cytokine is administered to the subject after a prolonged period of time (e.g., e.g., at least 2 weeks, 3 weeks, 4 weeks, 6 weeks, 8 weeks, 10 weeks, or more) after administration of the first population of MSPs.

In some embodiments of any of the aforesaid methods or uses, the disease, disorder, or condition is associated with a tumor antigen, e.g., a tumor antigen described herein, selected from a proliferative disease such as a cancer or malignancy or a precancerous condition such as a myelodysplasia, a myelodysplastic syndrome or a preleukemia, or is a non-cancer related indication associated with expression of a tumor antigen described herein. In some embodiments, the disease is a cancer described herein, e.g., a cancer described herein as being associated with a target described herein. In some embodiments, the disease is a hematologic cancer. In some embodiments, the hematologic cancer is leukemia. In some embodiments, the cancer is selected from the group consisting of one or more acute leukemias including but not limited to B-cell acute lymphoid leukemia (“BALL”), T-cell acute lymphoid leukemia (“TALL”), acute lymphoid leukemia (ALL); one or more chronic leukemias including but not limited to chronic myelogenous leukemia (CML), chronic lymphocytic leukemia (CLL); additional hematologic cancers or hematologic conditions including, but not limited to B cell prolymphocytic leukemia, blastic plasmacytoid dendritic cell neoplasm, Burkitt©lymphoma, diffuse large B cell lymphoma, follicular lymphoma, hairy cell leukemia, small cell- or a large cell-follicular lymphoma, malignant lymphoproliferative conditions, MALT lymphoma, mantle cell lymphoma, Marginal zone lymphoma, multiple myeloma, myelodysplasia and myelodysplastic syndrome, non-Hodgkin lymphoma, Hodgkin lymphoma, plasmablastic lymphoma, plasmacytoid dendritic cell neoplasm, Waldenstrom macroglobulinemia, and “preleukemia” which are a diverse collection of hematological conditions united by ineffective production (or dysplasia) of myeloid blood cells, and to disease associated with expression of a tumor antigen described herein include, but not limited to, atypical and/or non-classical cancers, malignancies, precancerous conditions or proliferative diseases expressing a tumor antigen as described herein; and any combination thereof. In another embodiment, the disease associated with a tumor antigen described herein is a solid tumor.

In embodiments, the cancer is selected from the group consisting of colon cancer, rectal cancer, renal-cell carcinoma, liver cancer, non-small cell carcinoma of the lung, cancer of the small intestine, cancer of the esophagus, melanoma, bone cancer, pancreatic cancer, skin cancer, cancer of the head or neck, cutaneous or intraocular malignant melanoma, uterine cancer, ovarian cancer, rectal cancer, cancer of the anal region, stomach cancer, testicular cancer, uterine cancer, carcinoma of the fallopian tubes, carcinoma of the endometrium, carcinoma of the cervix, carcinoma of the vagina, carcinoma of the vulva, Hodgkins Disease, non-Hodgkins lymphoma, cancer of the endocrine system, cancer of the thyroid gland, cancer of the parathyroid gland, cancer of the adrenal gland, sarcoma of soft tissue, cancer of the urethra, cancer of the penis, solid tumors of childhood, cancer of the bladder, cancer of the kidney or ureter, carcinoma of the renal pelvis, neoplasm of the central nervous system (CNS), primary CNS lymphoma, tumor angiogenesis, spinal axis tumor, brain stem glioma, pituitary adenoma, Kaposis sarcoma, epidermoid cancer, squamous cell cancer, T-cell lymphoma, environmentally induced cancers, combinations of said cancers, and metastatic lesions of said cancers.

In some embodiments, a cancer that can be treated with CAR-expressing cell of the present invention is multiple myeloma. Generally, myeloma cells are thought to be negative for a cancer associate antigen as described herein expression by flow cytometry. Thus, in some embodiments, a CD19 CAR, e.g., as described herein, may be used to target myeloma cells. In some embodiments, cars of the present invention therapy can be used in combination with one or more additional therapies, e.g., lenalidomide treatment.

In various aspects, the immune effector cells (e.g., T cells, NK cells) generated by the methods described herein and administered to the patient, or their progeny, persist in the patient for at least four months, five months, six months, seven months, eight months, nine months, ten months, eleven months, twelve months, thirteen months, fourteen month, fifteen months, sixteen months, seventeen months, eighteen months, nineteen months, twenty months, twenty-one months, twenty-two months, twenty-three months, two years, three years, four years, or five years after administration of the T cell or NK cell to the patient.

The invention also includes a type of cellular therapy where immune effector cells (e.g., T cells, NK cells) are modified, e.g., by in vitro or in vivo transcribed RNA, to transiently express a chimeric antigen receptor (CAR). The resultant cells are able to kill tumor cells in the subject or patient. Thus, in various aspects, the immune effector cells (e.g., T cells, NK cells) are present for less than one month, e.g., three weeks, two weeks, one week, after administration of the compositions as described herein.

Without wishing to be bound by any particular theory, the anti-tumor immunity response elicited by the CAR-modified immune effector cells (e.g., T cells, NK cells) may be an active or a passive immune response, or alternatively may be due to a direct vs indirect immune response. In some aspects, the CAR transduced immune effector cells (e.g., T cells, NK cells) exhibit specific proinflammatory cytokine secretion and potent cytolytic activity in response to human cancer cells expressing the a cancer associate antigen as described herein, resist soluble a cancer associate antigen as described herein inhibition, mediate bystander killing and mediate regression of an established human tumor. For example, antigen-less tumor cells within a heterogeneous field of a cancer associate antigen as described herein-expressing tumor may be susceptible to indirect destruction by a cancer associate antigen as described herein-redirected immune effector cells (e.g., T cells, NK cells) that has previously reacted against adjacent antigen-positive cancer cells.

In some aspects, the fully human CAR-modified immune effector cells (e.g., T cells, NK cells) of the invention may be a type of vaccine for ex vivo immunization and/or in vivo therapy in a mammal. In some aspects, the mammal is a human.

In some aspects the CAR-expressing cells of the inventions may be used to treat a proliferative disease such as a cancer or malignancy or is a precancerous condition such as a myelodysplasia, a myelodysplastic syndrome or a preleukemia. Further a disease associated with a cancer associate antigen as described herein expression include, but not limited to, e.g., atypical and/or non-classical cancers, malignancies, precancerous conditions or proliferative diseases expressing a cancer associated antigen as described herein. Non-cancer related indications associated with expression of a cancer associate antigen as described herein include, but are not limited to, e.g., autoimmune disease, (e.g., lupus), inflammatory disorders (allergy and asthma) and transplantation.

In some aspects the CAR-expressing cells of the inventions may be used to treat an autoimmune disease, an inflammatory disease, or transplant. Exemplary autoimmune diseases include but are not limited to Addison's disease, agammaglobulinemia, alopecia areata, amyloidosis, ankylosing spondylitis, anti-GBM/anti-TBM nephritis, antiphospholipid syndrome (APS), autoimmune hepatitis, autoimmune inner ear disease (AIED), axonal & neuronal neuropathy (AMAN), Behcet's disease, Bullous pemphigoid, Castleman disease (CD), Celiac disease, Chagas disease, chronic inflammatory demyelinating polyneuropathy (CIDP), chronic recurrent multifocal osteomyelitis (CRMO), Churg-Strauss, Cicatricial pemphigoid/benign mucosal pemphigoid, Cogan's syndrome, Cold agglutinin disease, Congenital heart block, Coxsackie myocarditis, CREST syndrome, Crohn's disease, dermatitis herpetiformis, dermatomyositis, Devic's disease (neuromyelitis optica), Discoid lupus, Dressler's syndrome, endometriosis, eosinophilic esophagitis (EoE), eosinophilic fasciitis, erythema nodosum, essential mixed cryoglobulinemia, Evans syndrome, fibromyalgia, fibrosing alveolitis, giant cell arteritis (temporal arteritis), giant cell myocarditis, Glomerulonephritis, Goodpasture's syndrome, Granulomatosis with Polyangiitis, Graves' disease, Guillain-Barre syndrome, Hashimoto's thyroiditis, hemolytic anemia, Henoch-Schonlein purpura (HSP), herpes gestationis or pemphigoid gestationis (PG), hypogammalglobulinemia, IgA nephropathy, IgG4-related sclerosing disease, inclusion body myositis (IBM), interstitial cystitis (IC), juvenile arthritis, juvenile diabetes (Type 1 diabetes), juvenile myositis (JM), Kawasaki disease, Lambert-Eaton syndrome, leukocytoclastic vasculitis, Lichen planus, Lichen sclerosus, Ligneous conjunctivitis, linear IgA disease (LAD), lupus, Lyme disease chronic, Meniere's disease, microscopic polyangiitis (MPA), mixed connective tissue disease (MCTD), Mooren's ulcer, Mucha-Habermann disease, multiple sclerosis (MS), Myasthenia gravis, Myositis, Narcolepsy, Neuromyelitis optica, neutropenia, ocular cicatricial pemphigoid, optic neuritis, palindromic rheumatism (PR), PANDAS (Pediatric Autoimmune Neuropsychiatric Disorders Associated with Streptococcus), paraneoplastic cerebellar degeneration (PCD), Paroxysmal nocturnal hemoglobinuria (PNH), Parry Romberg syndrome, Pars planitis (peripheral uveitis), Parsonnage-Turner syndrome, Pemphigus, peripheral neuropathy, Perivenous encephalomyelitis, pernicious anemia (PA), POEMS syndrome (polyneuropathy, organomegaly, endocrinopathy, monoclonal gammopathy, skin changes), polyarteritis nodosa, polymyalgia rheumatica, polymyositis, postmyocardial infarction syndrome, postpericardiotomy syndrome, primary biliary cirrhosis, primary sclerosing cholangitis, progesterone dermatitis, psoriasis, psoriatic arthritis, pure red cell aplasia (PRCA), pyoderma gangrenosum, Raynaud's phenomenon, Reactive Arthritis, Reflex sympathetic dystrophy, Reiter's syndrome, relapsing polychondritis, restless legs syndrome (RLS), retroperitoneal fibrosis, rheumatic fever, rheumatoid arthritis (RA), sarcoidosis, Schmidt syndrome, scleritis, scleroderma, Sjogren's syndrome, sperm & testicular autoimmunity, Stiff person syndrome (SPS), subacute bacterial endocarditis (SBE), Susac's syndrome, sympathetic ophthalmia (SO), Takayasu's arteritis, temporal arteritis/Giant cell arteritis, thrombocytopenic purpura (TTP), Tolosa-Hunt syndrome (THS), transverse myelitis, type 1 diabetes, ulcerative colitis (UC), undifferentiated connective tissue disease (UCTD), uveitis, vasculitis, vitiligo, or Wegener's granulomatosis (Granulomatosis with Polyangiitis (GPA)). In some embodiments, the CAR binds to a B cell antigen, e.g., CD 19, CD20, CD22, CD123, FcRn5, FcRn2, BCMA, CS-1 and CD138.

The CAR-modified immune effector cells (e.g., T cells, NK cells) of the present invention may be administered either alone, or as a pharmaceutical composition in combination with diluents and/or with other components such as IL-2 or other cytokines or cell populations.

Hematologic Cancer

Hematological cancer conditions are the types of cancer such as leukemia, lymphoma, and malignant lymphoproliferative conditions that affect blood, bone marrow and the lymphatic system.

Leukemia can be classified as acute leukemia and chronic leukemia. Acute leukemia can be further classified as acute myelogenous leukemia (AML) and acute lymphoid leukemia (ALL). Chronic leukemia includes chronic myelogenous leukemia (CML) and chronic lymphoid leukemia (CLL). Other related conditions include myelodysplastic syndromes (MDS, formerly known as “preleukemia”) which are a diverse collection of hematological conditions united by ineffective production (or dysplasia) of myeloid blood cells and risk of transformation to AML. Lymphoma is a group of blood cell tumors that develop from lymphocytes. Exemplary lymphomas include non-Hodgkin lymphoma and Hodgkin lymphoma.

The present invention also provides methods for inhibiting the proliferation or reducing a cancer associated antigen as described herein, the methods comprising contacting a population of cells comprising a cancer associated antigen as described herein with a composition comprising a mesoporous silica particles and a viral vector. In a specific aspect, the MSPs are surface modified as described herein. In other embodiments, the viral vector comprises an expression vector comprising a recombinant polynucleotide comprising an expression control sequence operatively linked to a nucleotide sequence to be expressed. Exemplary nucleotide sequences express a chimeric antigen receptor (CAR), engineered TCR, cytokines, chemokines, shRNA to block an inhibitory molecule, or mRNA to induce expression of a protein. In some aspects, a CAR-expressing T cell or NK cell of the invention reduces the quantity, number, amount or percentage of cells and/or cancer cells by at least 25%, at least 30%, at least 40%, at least 50%, at least 65%, at least 75%, at least 85%, at least 95%, or at least 99% in a subject with or animal model for myeloid leukemia or another cancer associated with a cancer associated antigen as described herein-expressing cells relative to a negative control. In some aspects, the subject is a human.

### Combination Therapies

Administered “in combination”, as used herein, means that two (or more) different treatments are delivered to the subject during the course of the subject [symbol] affliction with the disorder, e.g., the two or more treatments are delivered after the subject has been diagnosed with the disorder and before the disorder has been cured or eliminated or treatment has ceased for other reasons. In some embodiments, the delivery of one treatment is still occurring when the delivery of the second begins, so that there is overlap in terms of administration. This is sometimes referred to herein as “simultaneous” or “concurrent delivery”. In other embodiments, the delivery of one treatment ends before the delivery of the other treatment begins. In some embodiments of either case, the treatment is more effective because of combined administration. For example, the second treatment is more effective, e.g., an equivalent effect is seen with less of the second treatment, or the second treatment reduces symptoms to a greater extent, than would be seen if the second treatment were administered in the absence of the first treatment, or the analogous situation is seen with the first treatment. In some embodiments, delivery is such that the reduction in a symptom, or other parameter related to the disorder is greater than what would be observed with one treatment delivered in the absence of the other. The effect of the two treatments can be partially additive, wholly additive, or greater than additive. The delivery can be such that an effect of the first treatment delivered is still detectable when the second is delivered.

In some embodiments, the methods or uses are carried out in combination with an agent that increases the efficacy of the immune effector cell, e.g., an agent as described herein. In some embodiments of the methods or uses described herein, the mesoporous silica rod composition is administered in combination with an agent that increases the efficacy of the immune effector cell, e.g., one or more of a protein phosphatase inhibitor, a kinase inhibitor, a cytokine, an inhibitor of an immune inhibitory molecule; or an agent that decreases the level or activity of a T_{REG} cell.

In some embodiments of the methods or uses described herein, the protein phosphatase inhibitor is a SHP-1 inhibitor and/or an SHP-2 inhibitor.

In other embodiments of the methods or uses described herein, kinase inhibitor is chosen from one or more of a CDK4 inhibitor, a CDK4/6 inhibitor (e.g., palbociclib), a BTK inhibitor (e.g., ibrutinib or RN-486), an mTOR inhibitor (e.g., rapamycin or everolimus (RAD001), an MNK inhibitor, or a dual P13K/mTOR inhibitor. In some embodiments, the BTK inhibitor does not reduce or inhibit the kinase activity of interleukin-2-inducible kinase (ITK).

In other embodiments of the methods or uses described herein, the agent that inhibits the immune inhibitory molecule comprises an antibody or antibody fragment, an inhibitory nucleic acid, a clustered regularly interspaced short palindromic repeats (CRISPR), a transcription-activator like effector nuclease (TALEN), or a zinc finger endonuclease (ZFN) that inhibits the expression of the inhibitory molecule.

In other embodiments of the methods or uses described herein, the agent that decreases the level or activity of the TREG cells is chosen from cyclophosphamide, anti-GITR antibody, CD25-depletion, or a combination thereof.

In some embodiments of the methods or uses described herein, the immune inhibitory molecule is selected from the group consisting of PD1, PD-L1, CTLA-4, TIM-3, LAG-3, VISTA, BTLA, TIGIT, LAIR1, CD160, 2B4, TGFR beta, CEACAM-1, CEACAM-3, and CEACAM-5.

In other embodiments, the agent that inhibits the inhibitory molecule comprises a first polypeptide comprising an inhibitory molecule or a fragment thereof and a second polypeptide that provides a positive signal to the cell, and wherein the first and second polypeptides are expressed on the CAR-containing immune cells, wherein (i) the first polypeptide comprises PD1, PD-L1, CTLA-4, TIM-3, LAG3, VISTA, BTLA, TIGIT, LAIR1, CD160, 2B4, TGFR beta, CEACAM-1, CEACAM-3, and CEACAM-5 or a fragment thereof, and/or (ii) the second polypeptide comprises an intracellular signaling domain comprising a primary signaling domain and/or a costimulatory signaling domain. In some embodiments, the primary signaling domain comprises a functional domain of CD3 zeta; and/or the costimulatory signaling domain comprises a functional domain of a protein selected from 41BB, CD27, and CD28. In other embodiments, cytokine is chosen from IL-7, IL-15, or IL-21, or combinations thereof.

In other embodiments, the immune effector cell comprising the CAR molecule and a second, e.g., any of the combination therapies disclosed herein (e.g., the agent that that increases the efficacy of the immune effector cell) are administered substantially simultaneously or sequentially.

In other embodiments, the immune cell comprising the CAR molecule is administered in combination with a molecule that targets GITR and/or modulates GITR function. In some embodiments, the molecule targeting GITR and/or modulating GITR function is administered prior to the CAR-expressing cell or population of cells, or prior to apheresis.

In some embodiments, lymphocyte infusion, for example allogeneic lymphocyte infusion, is used in the treatment of the cancer, wherein the lymphocyte infusion comprises at least one CAR-expressing cell of the present invention. In some embodiments, autologous lymphocyte infusion is used in the treatment of the cancer, wherein the autologous lymphocyte infusion comprises at least one CAR-expressing cell described herein.

In some embodiments, the cell is a T cell and the T cell is diaglycerol kinase (DGK) deficient. In some embodiments, the cell is a T cell and the T cell is Ikaros deficient. In some embodiments, the cell is a T cell and the T cell is both DGK and Ikaros deficient.

In embodiments of any of the aforesaid methods or uses, there may be a further administration of an agent that treats the disease associated with expression of the tumor antigen, e.g., any of the second or third therapies disclosed herein. Additional exemplary combinations include one or more of the following.

In another embodiment, there may be a further administration of another agent, e.g., a kinase inhibitor and/or checkpoint inhibitor described herein. For example, there may be a further administration of an agent which enhances the activity of a CAR-expressing cell.

For example, in some embodiments, the agent that enhances the activity of a CAR-expressing cell can be an agent which inhibits an inhibitory molecule (e.g., an immune inhibitor molecule). Examples of inhibitory molecules include PD1, PD-L1, CTLA-4, TIM-3, CEACAM (e.g., CEACAM-1, CEACAM-3 and/or CEACAM-5), LAG-3, VISTA, BTLA, TIGIT, LAIR1, CD160, 2B4, and TGFR beta.

In some embodiments, the agent that inhibits the inhibitory molecule is an inhibitory nucleic acid is a dsRNA, a siRNA, or a shRNA. In embodiments, the inhibitory nucleic acid is linked to the nucleic acid that encodes a component of the CAR molecule. For example, the inhibitory molecule can be expressed on the CAR-expressing cell.

In another embodiment, the agent which inhibits an inhibitory molecule, e.g., is a molecule described herein, e.g., an agent that comprises a first polypeptide, e.g., an inhibitory molecule, associated with a second polypeptide that provides a positive signal to the cell, e.g., an intracellular signaling domain described herein. In some embodiments, the agent comprises a first polypeptide, e.g., of an inhibitory molecule such as PD-1, PD-L1, CTLA-4, TIM-3, CEACAM (e.g., CEACAM-1, CEACAM-3 and/or CEACAM-5), LAG-3, VISTA, BTLA, TIGIT, LAIR1, CD160, 2B4, or TGFR beta, or a fragment of any of these (e.g., at least a portion of the extracellular domain of any of these), and a second polypeptide which is an intracellular signaling domain described herein (e.g., comprising a costimulatory domain (e.g., 41BB, CD27 or CD28, e.g., as described herein) and/or a primary signaling domain (e.g., a CD3 zeta signaling domain described herein). In some embodiments, the agent comprises a first polypeptide of PD1 or a fragment thereof (e.g., at least a portion of the extracellular domain of PD1), and a second polypeptide of an intracellular signaling domain described herein (e.g., a CD28 signaling domain described herein and/or a CD3 zeta signaling domain described herein).

In some embodiments, the CAR-expressing immune effector cell of the present invention, e.g., T cell or NK cell, is administered to a subject that has received a previous stem cell transplantation, e.g., autologous stem cell transplantation.

In some embodiments, the CAR-expressing immune effector cell of the present invention, e.g., T cell or NK cells, is administered to a subject that has received a previous dose of melphalan.

In some embodiments, the cell expressing a CAR molecule, e.g., a CAR molecule described herein, is administered in combination with an agent that increases the efficacy of a cell expressing a CAR molecule, e.g., an agent described herein.

In some embodiments, the cell expressing a CAR molecule, e.g., a CAR molecule described herein, is administered in combination with an agent that ameliorates one or more side effect associated with administration of a cell expressing a CAR molecule, e.g., an agent described herein.

In some embodiments, the cell expressing a CAR molecule, e.g., a CAR molecule described herein, is administered in combination with an agent that treats the disease associated with a cancer associated antigen as described herein, e.g., an agent described herein.

In some embodiments, a cell expressing two or more CAR molecules, e.g., as described herein, is administered to a subject in need thereof to treat cancer. In some embodiments, a population of cells including a CAR expressing cell, e.g., as described herein, is administered to a subject in need thereof to treat cancer.

In some embodiments of the methods or uses described herein, the CAR molecule is administered in combination with another agent. In some embodiments, the agent can be a kinase inhibitor, e.g., a CDK4/6 inhibitor, a BTK inhibitor, an mTOR inhibitor, a MNK inhibitor, or a dual PI3K/mTOR inhibitor, and combinations thereof. In some embodiments, the kinase inhibitor is a CDK4 inhibitor, e.g., a CDK4 inhibitor described herein, e.g., a CD4/6 inhibitor, such as, e.g., 6-Acetyl-8-cyclopentyl-5-methyl-2-(5-piperazin-1-yl-pyridin-2-ylamino)-8H-pyrido[2,3-d]pyrimidin-7-one, hydrochloride (also referred to as palbociclib or PD0332991). In some embodiments, the kinase inhibitor is a BTK inhibitor, e.g., a BTK inhibitor described herein, such as, e.g., ibrutinib. In some embodiments, the kinase inhibitor is an mTOR inhibitor, e.g., an mTOR inhibitor described herein, such as, e.g., rapamycin, a rapamycin analog, OSI-027. The mTOR inhibitor can be, e.g., an mTORC1 inhibitor and/or an mTORC2 inhibitor, e.g., an mTORC1 inhibitor and/or mTORC2 inhibitor described herein. In some embodiments, the kinase inhibitor is a MNK inhibitor, e.g., a MNK inhibitor described herein, such as, e.g., 4-amino-5-(4-fluoroanilino)-pyrazolo [3,4-d] pyrimidine. The MNK inhibitor can be, e.g., a MNK1a, MNK1b, MNK2a and/or MNK2b inhibitor. The dual PI3K/mTOR inhibitor can be, e.g., PF-04695102.

In some embodiments of the methods or uses described herein, the kinase inhibitor is a CDK4 inhibitor selected from aloisine A; flavopiridol or HMR-1275, 2-(2-chlorophenyl)-5,7-dihydroxy-8-[(3S,4R)-3-hydroxy-1-methyl-4-piperidinyl]-4-chromenone; crizotinib (PF-02341066; 2-(2-Chlorophenyl)-5,7-dihydroxy-8-[(2R,3S)-2-(hydroxymethyl)-1-methyl-3-pyrrolidinyl]-4H-1-benzopyran-4-one, hydrochloride (P276-00); 1-methyl-5-[[2-[5-(trifluoromethyl)-1H-imidazol-2-yl]-4-pyridinyl]oxy]-N-[4-(trifluoromethyl)phenyl]-1H-benzimidazol-2-amine (RAF265); indisulam (E7070); roscovitine (CYC202); palbociclib (PD0332991); dinaciclib (SCH727965); N-[5-[[(5-tert-butyloxazol-2-yl)methyl]thio]thiazol-2-yl]piperidine-4-carboxamide (BMS 387032); 4-[[9-chloro-7-(2,6-difluorophenyl)-5H-pyrimido[5,4-d][2]benzazepin-2-yl]amino]-benzoic acid (MLN8054); 5-[3-(4,6-difluoro-1H-benzimidazol-2-yl)-1H-indazol-5-yl]-N-ethyl-4-methyl-3-pyridinemethanamine (AG-024322); 4-(2,6-dichlorobenzoylamino)-1H-pyrazole-3-carboxylic acid N-(piperidin-4-yl)amide (AT7519); 4-[2-methyl-1-(1-methylethyl)-1H-imidazol-5-yl]-N-[4-(methylsulfonyl)phenyl]-2-pyrimidinamine (AZD5438); and XL281 (BMS908662).

In some embodiments of the methods or uses described herein, the kinase inhibitor is a CDK4 inhibitor, e.g., palbociclib (PD0332991), and the palbociclib is administered at a dose of about 50 mg, 60 mg, 70 mg, 75 mg, 80 mg, 90 mg, 100 mg, 105 mg, 110 mg, 115 mg, 120 mg, 125 mg, 130 mg, 135 mg (e.g., 75 mg, 100 mg or 125 mg) daily for a period of time, e.g., daily for 14-21 days of a 28 day cycle, or daily for 7-12 days of a 21 day cycle. In some embodiments, 1, 2, 3, 4, 5, 6, 7, 8, 9, 10, 11, 12 or more cycles of palbociclib are administered.

In some embodiments of the methods or uses described herein, the kinase inhibitor is a BTK inhibitor selected from ibrutinib (PCI-32765); GDC-0834; RN-486; CGI-560; CGI-1764; HM-71224; CC-292; ONO-4059; CNX-774; and LFM-A13. In some embodiments, the BTK inhibitor does not reduce or inhibit the kinase activity of interleukin-2-inducible kinase (ITK), and is selected from GDC-0834; RN-486; CGI-560; CGI-1764; HM-71224; CC-292; ONO-4059; CNX-774; and LFM-A13.

In some embodiments of the methods or uses described herein, the kinase inhibitor is a BTK inhibitor, e.g., ibrutinib (PCI-32765), and the ibrutinib is administered at a dose of about 250 mg, 300 mg, 350 mg, 400 mg, 420 mg, 440 mg, 460 mg, 480 mg, 500 mg, 520 mg, 540 mg, 560 mg, 580 mg, 600 mg (e.g., 250 mg, 420 mg or 560 mg) daily for a period of time, e.g., daily for 21 day cycle, or daily for 28 day cycle. In some embodiments, 1, 2, 3, 4, 5, 6, 7, 8, 9, 10, 11, 12 or more cycles of ibrutinib are administered.

In some embodiments of the methods or uses described herein, the kinase inhibitor is a BTK inhibitor that does not inhibit the kinase activity of ITK, e.g., RN-486, and RN-486 is administered at a dose of about 100 mg, 110 mg, 120 mg, 130 mg, 140 mg, 150 mg, 160 mg, 170 mg, 180 mg, 190 mg, 200 mg, 210 mg, 220 mg, 230 mg, 240 mg, 250 mg (e.g., 150 mg, 200 mg or 250 mg) daily for a period of time, e.g., daily a 28 day cycle. In some embodiments, 1, 2, 3, 4, 5, 6, 7, or more cycles of RN-486 are administered.

In some embodiments of the methods or uses described herein, the kinase inhibitor is an mTOR inhibitor selected from temsirolimus; ridaforolimus (1R,2R,4S)-4-[(2R)-2 [(1R,9S,12S,15R,16E,18R,19R,21R,23S,24E,26E,28Z,30S,32S,35R)-1,18-dihydroxy-19,30-dimethoxy-15,17,21,23, 29,35-hexamethyl-2,3,10,14,20-pentaoxo-11,36-dioxa-4-azatricyclo[30.3.1.04° 9] hexatriaconta-16,24,26,28-tetraen-12-yl]propyl]-2-methoxycyclohexyl dimethylphosphinate, also known as AP23573 and MK8669; everolimus (RAD001); rapamycin (AY22989); simapimod; (5-{2,4-bis[(3S)-3-methylmorpholin-4-yl]pyrido[2,3-d]pyrimidin-7-yl}-2-methoxyphenyl)methanol (AZD8055); 2-amino-8-[trans-4-(2-hydroxyethoxy)cyclohexyl]-6-(6-methoxy-3-pyridinyl)-4-methyl-pyrido[2,3-d]pyrimidin-7(8H)-one (PF04691502); and N²-[1,4-dioxo-4-[[4-(4-oxo-8-phenyl-4H-1-benzopyran-2-yl)morpholinium-4-yl]methoxy]butyl]-L-arginylglycyl-L-α-aspartylL-serine- (SEQ ID NO: 692), inner salt (SF1126); and XL765.

In some embodiments of the methods or uses described herein, the kinase inhibitor is an mTOR inhibitor, e.g., rapamycin, and the rapamycin is administered at a dose of about 3 mg, 4 mg, 5 mg, 6 mg, 7 mg, 8 mg, 9 mg, 10 mg (e.g., 6 mg) daily for a period of time, e.g., daily for 21 day cycle, or daily for 28 day cycle. In some embodiments, 1, 2, 3, 4, 5, 6, 7, 8, 9, 10, 11, 12 or more cycles of rapamycin are administered. In some embodiments, the kinase inhibitor is an mTOR inhibitor, e.g., everolimus and the everolimus is administered at a dose of about 2 mg, 2.5 mg, 3 mg, 4 mg, 5 mg, 6 mg, 7 mg, 8 mg, 9 mg, 10 mg, 11 mg, 12 mg, 13 mg, 14 mg, 15 mg (e.g., 10 mg) daily for a period of time, e.g., daily for 28 day cycle. In some embodiments, 1, 2, 3, 4, 5, 6, 7, 8, 9, 10, 11, 12 or more cycles of everolimus are administered.

In some embodiments of the methods or uses described herein, the kinase inhibitor is an MNK inhibitor selected from CGP052088; 4-amino-3-(p-fluorophenylamino)-pyrazolo [3,4-d]pyrimidine (CGP57380); cercosporamide; ETC-1780445-2; and 4-amino-5-(4-fluoroanilino)-pyrazolo [3,4-d] pyrimidine.

In some embodiments of the methods or uses described herein, the kinase inhibitor is a dual phosphatidylinositol 3-kinase (PI3K) and mTOR inhibitor selected from 2-Amino-8-[trans-4-(2-hydroxyethoxy)cyclohexyl]-6-(6-methoxy-3-pyridinyl)-4-methyl-pyrido[2,3-d]pyrimidin-7(8H)-one (PF-04691502); N-[4-[[4-(Dimethylamino)-1-piperidinyl]carbonyl]phenyl]-N- [4-(4,6-di-4-morpholinyl-1,3,5-triazin-2-yl)phenyl]urea (PF-05212384, PKI-587); 2-Methyl-2-{4-[3-methyl-2-oxo-8-(quinolin-3-yl)-2,3-dihydro-1H-imidazo[4,5-c]quinolin-1-yl]phenyl}propanenitrile (BEZ-235); apitolisib (GDC-0980, RG7422); 2,4-Difluoro-N-{2-(methyloxy)-5-[4-(4-pyridazinyl)-6-quinolinyl]-3-pyridinyl}benzenesulfonamide (GSK2126458); 8-(6-methoxypyridin-3-yl)-3-methyl-1-(4-(piperazin-1-yl)-3-(trifluoromethyl)phenyl)-1H-imidazo[4,5-c]quinolin-2(3H)-one Maleic acid (NVP-BGT226); 3-[4-(4-Morpholinylpyrido[3,2,4,5]furo[3,2-d]pyrimidin-2-yl]phenol (PI-103); 5-(9-isopropyl-8-methyl-2-morpholino-9H-purin-6-yl)pyrimidin-2-amine (VS-5584, SB2343); and N-[2-[(3,5-Dimethoxyphenyl)amino]quinoxalin-3-yl]-4-[(4-methyl-3-methoxyphenyl)carbonyl]aminophenylsulfonamide (XL765).

In some embodiments of the methods or uses described herein, there may be a further administration of a protein tyrosine phosphatase inhibitor, e.g., a protein tyrosine phosphatase inhibitor described herein. In some embodiments, the protein tyrosine phosphatase inhibitor is an SHP-1 inhibitor, e.g., an SHP-1 inhibitor described herein, such as, e.g., sodium stibogluconate. In some embodiments, the protein tyrosine phosphatase inhibitor is an SHP-2 inhibitor.

In some embodiments of the methods or uses described herein, there may be a further administration of another agent, and the agent is a cytokine. The cytokine can be, e.g., IL-7, IL-15, IL-21, or a combination thereof. In another embodiment, the CAR molecule is administered in combination with a checkpoint inhibitor, e.g., a checkpoint inhibitor described herein. For example, in some embodiments, the check point inhibitor inhibits an inhibitory molecule selected from PD-1, PD-L1, CTLA-4, TIM-3, CEACAM (e.g., CEACAM-1, CEACAM-3 and/or CEACAM-5), LAG-3, VISTA, BTLA, TIGIT, LAIR1, CD160, 2B4, and TGFR beta.

In other embodiments of the methods or uses described herein, there may be a further administration of an agent that ameliorates one or more side effects associated with a cell expressing a CAR molecule. Side effects associated with the CAR-expressing cell can be chosen from cytokine release syndrome (CRS) or hemophagocytic lymphohistiocytosis (HLH).

The present invention also provides methods for preventing, treating and/or managing a disease associated with a cancer associated antigen as described herein-expressing cells (e.g., a hematologic cancer or atypical cancer expressing a cancer associated antigen as described herein), the method comprising administering to a subject a composition comprising a first population of mesoporous silica particles and a viral vector, and wherein the viral vector comprises an expression vector comprising a recombinant polynucleotide comprising an expression control sequence operatively linked to a nucleotide sequence that expresses a chimeric antigen receptor (CAR) that is engineered to target the tumor antigen. In some aspects, the subject is a human. Non-limiting examples of disorders associated with a cancer associated antigen as described herein-expressing cells include autoimmune disorders (such as lupus), inflammatory disorders (such as allergies and asthma) and cancers (such as hematological cancers or atypical cancers expressing a cancer associated antigen as described herein).

The present invention also provides methods for preventing, treating and/or managing a disease associated with a cancer associated antigen as described herein-expressing cells, the methods comprising administering to a subject a composition comprising a first population of mesoporous silica particles and a viral vector, and wherein the viral vector comprises an expression vector comprising a recombinant polynucleotide comprising an expression control sequence operatively linked to a nucleotide sequence that expresses a chimeric antigen receptor (CAR) that is engineered to target the tumor antigen. In some aspects, the subject is a human.

The present invention provides methods for preventing relapse of cancer associated with a cancer associated antigen as described herein-expressing cells, the methods comprising administering to a subject a composition comprising a first population of mesoporous silica particles and a viral vector, and wherein the viral vector comprises an expression vector comprising a recombinant polynucleotide comprising an expression control sequence operatively linked to a nucleotide sequence that expresses a chimeric antigen receptor (CAR) that is engineered to target the tumor antigen.

When “an immunologically effective amount,” “an anti-tumor effective amount,” “a tumor-inhibiting effective amount,” or “therapeutic amount” is indicated, the precise amount of the compositions of the present invention to be administered can be determined by a physician with consideration of individual differences in age, weight, tumor size, extent of infection or metastasis, and condition of the patient (subject).

In some aspects, it may be desired to administer activated immune effector cells (e.g., T cells, NK cells) to a subject and then subsequently redraw blood (or have an apheresis performed), activate and expand immune effector cells (e.g., T cells, NK cells) according to the present invention, and reinfuse the patient with these activated and expanded immune effector cells (e.g., T cells, NK cells). This process can be carried out multiple times every few weeks. In some aspects, immune effector cells (e.g., T cells, NK cells) can be activated from blood draws of from 10 cc to 400 cc. In some aspects, immune effector cells (e.g., T cells, NK cells) are activated from blood draws of 20 cc, 30 cc, 40 cc, 50 cc, 60 cc, 70 cc, 80 cc, 90 cc, or 100 cc.

The administration of the subject compositions may be carried out in any convenient manner, including by aerosol inhalation, injection, ingestion, transfusion, implantation or transplantation. The compositions described herein may be administered to a patient trans arterially, subcutaneously, intradermally, intratumorally, intranodally, intramedullary, intramuscularly, by intravenous (i.v.) injection, or intraperitoneally. In some aspects, the MSP (e.g., MSR) compositions of the present invention are administered to a patient by intradermal or subcutaneous injection. In some aspects, the T cell compositions of the present invention are administered parenterally. The term “parenteral” administration of an T cell composition includes, e.g., intrathecal, epidural, intracranial, subcutaneous (s.c.), intravenous (i.v.), intramuscular (i.m.), or intrasternal injection, intratumoral, or infusion techniques. In particular embodiments, the T cell composition is administered intravenously. In some embodiments, the compositions of MSPs (e.g., MSRs) and viral vectors may be injected directly into a tumor, lymph node, or site of infection.

### EXAMPLES

### Example A. Synthesis and Post-Functionalization of Mesoporous Silica Particles

Unless otherwise noted, all reagents were obtained from commercial sources and used as is.

1. Exemplary Synthesis of Mesoporous Silica Particles

Poly(ethylene glycol)-block-poly(propylene glycol)-block-poly(ethylene glycol) avg Mn ˜5,800 (Pluronic P-123, 80.0 g, 487 mmol; Sigma) surfactant was dissolved in 3 L of 1.6M HCl at room temperature, heated to 40 degrees Celsius in a 5 L jacketed flask, and was mechanically stirred via and overhead stirrer at a rate of 0-600 rpm (but most commonly 300 rpm). Tetraethyl orthosilicate (TEOS, 184 mL, 826 mmol; Sigma) was added in one portion over <5 min and was heated at 40 degrees Celsius with maintained stirring for at least 2 hours but most commonly 20 hours. The resulting slurry was heated to 80-130 degrees Celsius (most commonly 100 degrees Celsius) for 6-72 hours (but most commonly 24 hours) for hydrothermal treatment before being cooled to room temperature. The slurry was filtered in a Buchner funnel and was washed with deionized water followed by ethanol and air dried at room temperature. The resulting silica material was calcined in a furnace with a slow ramp temperature from room temperature to 550 degrees Celsius over 8 hours and then maintaining at 550 degrees Celsius for another 8 hours before cooling to room temperature to afford 47 g of mesoporous silica particles.

Changes in the stir rate may have changes in the microparticle aspect ratio. Varying the conditions of the hydrothermal temperature and duration are common pore size controllers for mesoporous materials. For more information, see J. Chem. Educ. 2017, 94, 91-94 and references within.

Final mesoporous materials were characterized by light microscopy, Malvern Morphologi G3, scanning electron microscopy (SEM), thermal gravimetric analysis (TGA).

2. Post-Modification of Silica Microparticles

### Example 2(a): Diethyl Ethylphosphonate Functionalized Microparticles

Diethyl ethylphosphonate functionalized silica microparticles were prepared by a modified method reported in New J. Chem., 2014, 38, 3853, with some modifications. Diethylphosphatoethyltriethoxysilane (4.15 mL, 13.03 mmol) was added to a slurry of 2.0 g of mesoporous silica microparticles suspended in 300 mL of toluene. The slurry was stirred and refluxed at 110 degrees Celsius for 14 hours before cooling to room temperature and filtered. The particles were washed with deionized water followed by ethanol and then dried in an oven at 100 degrees Celsius for 20 hours to afford diethyl ethylphosphonate functionalized particles.

### Example 2(b): Ethylphosphonic Acid Functionalized Microparticles

Ethylphosphonic acid functionalized microparticles were prepared by a modified method to the procedure reported in New J. Chem., 2014, 38, 3853. Trimethylsilylchlorosilane (1.388 mL, 10.86 mmol) was added to a slurry of 2.0 g of diethyl ethylphosphonate functionalized microparticles suspended in 150 mL of toluene and heated to 110 degrees Celsius for 24 hours. The slurry was cooled to room temperature and filtered, washing with dionized water and ethanol before drying in a oven at 100 degrees Celsius for 24 hours. The mesoporous silica particles were then suspended in 100 mL of 12M HCl and heated to 100 degrees Celsius for 18 hours. The slurry was cooled to room temperature, filtered and washed with deionized water and ethanol before drying in an oven at 100 degrees Celsius for 24 hours to afford ethylphosphonic acid functionalized microparticles.

### Example 2(c): Propylamine Functionalized Microparticles

Propylamine functionalized microparticles were prepared by a modified method to the procedure reported in Langmuir 2015, 31, 6457-6462. (3-aminopropyl)trimethoxysilane (3.05 ml, 19.54 mmol; APTMS, Sigma) was added to a slurry of 3.0 grams of mesoporous silica microparticles in 150 mL of reagent grade ethanol. The slurry was refluxed at 75 degrees Celsius for 7 hours. After cooling to room temperature and the slurry was filtered and the particles were washed with deionized water followed by ethanol and then dried in an oven at 100 degrees Celsius for 24 hours.

### Example 2(d): Biotin Functionalized Microparticles

(+)-Biotin N-succinimidyl ester (246 mg, 0.720 mmol) was added to a slurry of 1.0 g of propylamine-functionalized microparticles in 10.0 mL of pH 7.4 adjusted PBS buffer and stirred at room temperature for 18 hours. The slurry was filtered and washed with deionized water and ethanol before drying in an oven at 100 degrees Celsius for 24 hours to afford Biotin-functionalized microparticles.

### Example 2(e): Biotin-PEG4 Functionalized Microparticles

PEG4-Biotin N-hydroxysuccinimide (106 mg, 0.180 mmol; ThermoFischer EZ-Link NHS-PEG4-biotin) was added to a slurry of 0.25 g of propylamine-functionalized microparticles in 2.5 mL of pH 7.4 adjusted PBS buffer and stirred at room temperature for 18 hours. The slurry was filtered and washed with deionized water and ethanol before drying in an oven at 100 degrees Celsius for 24 hours to afford Biotin-PEG4 functionalized microparticles.

### Example 2(f): 3(2-Pyridyldithio)Propionamido)Hexanoate Functionalized Microparticles

Succinimidyl 6-(3(2-pyridyldithio)propionamido)hexanoate (112 mg, 0.360 mmol; LC-SPDP, ThermoFischer) was added to a slurry of 0.50 g of propylamine-functionalized microparticles in 2.5 mL of pH 7.4 adjusted PBS buffer and stirred at room temperature for 18 hours. The slurry was filtered and washed with deionized water and ethanol before drying in an oven at 100 degrees Celsius for 24 hours to afford 3(2-pyridyldithio)propionamido)hexanoate-functionalized microparticles.

### Example 2(g): 4-Oxo-4-(Propylamino)Butanoic Acid Functionalized Microparticles

Succinic anhydride (4 g, 40.0 mmol) was added to a slurry of 1.0 g of propylamine-functionalized microparticles in anhydrous DMF and was stirred at room temperature for 24 hours. The slurry was filtered and washed with deionized water and ethanol before drying in an oven at 100 degrees Celsius for 24 hours to afford 4-oxo-4-(propylamino)butanoic acid functionalized microparticles.

### Example 2(h): Propyldiethylenetriamine Functionalized Microparticles

Trimethoxysilylpropyldiethylenetriamine (1.678 mL, 6.51 mmol) was added to 1.0 g of mesoporous silica microparticles were suspended in 150 mL of reagent grade ethanol. The slurry was stirred at 75 degrees Celsius for 7 hours. After cooling to room temperature and the slurry was filtered and the particles were washed with deionized water followed by ethanol and then dried in an oven at 100 degrees Celsius for 20 hours to afford propyldiethylenetriamine functionalized microparticles.

### Example 2(i): 3-Propyldihydrofuran-2,5-Dione Functionalized Microparticles (Succinic Anhydride)

3-(3-(triethoxysilyl)propyl)dihydrofuran-2,5-dione (4.94 mL, 17.37 mmol) was added to a slurry of 3.0 g of mesoporous silica microparticles in 300 mL of toluene. The slurry was heated to 110 degrees Celsius for 20 hours and was then cooled to room temperature, filtered and washed with dionizied water and ethanol. The functionalized microparticles were dried in an oven at 100 degrees Celsius for 24 hours.

### Example 2(j): Branched, Polyethylenimine Functionalized Microparticles

Polyethylenimine (25.1 g, 47.0 mmol; Branched, avg Mw ˜25,000, Sigma) was dissolved in 600 mL of anhydrous DMF and 6.0 g of 3-propyldihydrofuran-2,5-dione functionalized microparticles were added and stirred at room temperature for 20 hours. The slurry was filtered, and the particles were washed with deionized water followed by ethanol and then dried in an oven at 100 degrees Celsius for 20 hours to afford branched, polyethylenimine functionalized microparticles.

### Example 2(k): N,N,N-Trimethylpropan-1-Ammonium Functionalized Microparticles

Trimethoxysilylpropyltrimethylammonium chloride (3.61 mL, 6.51 mmol; 50% solution in methanol) was added to a slurry of 1.0 g mesoporous silica microparticles in 150 mL reagent ethanol and was heated to 75 degrees Celsius for 7 hours. After cooling to room temperature and the slurry was filtered and the particles were washed with deionized water followed by ethanol and then dried in an oven at 100 degrees Celsius for 20 hours to afford N,N,N-trimethylpropan-1-ammonium functionalized microparticles.

The procedure above was repeated at varying ratios of trimethoxysilylpropyltrimethylammonium chloride to silica microparticles (0.25 mmol trimethoxysilyltrimethylammonium chloride per gram of microparticles) to affect varying ratios of functional density.

### Example 2(1): Octyl Functionalized Microparticles

Triethyoxy(octyl)silane (2.05 mL, 6.51 mmol) was added to a slurry of 1.0 g mesoporous silica microparticles in 150 mL reagent ethanol and was heated to 75 degrees Celsius for 7 hours. After cooling to room temperature and the slurry was filtered and the particles were washed with deionized water followed by ethanol and then dried in an oven at 100 degrees Celsius for 20 hours to afford octyl functionalized microparticles.

### Example 2(m): Hexadecyl Functionalized Microparticles

Hexadecyltrimethoxysilane (2.54 mL, 6.51 mmol) was added to a slurry of 1.0 g mesoporous silica microparticles in 150 mL reagent ethanol and was heated to 75 degrees Celsius for 7 hours. After cooling to room temperature and the slurry was filtered and the particles were washed with deionized water followed by ethanol and then dried in an oven at 100 degrees Celsius for 20 hours to afford hexadecyl functionalized microparticles.

### Example 2(n): 11-Azidoundecyl Functionalized Microparticles

(11-azidoundecyl)trimethoxysilane (1.0 g, 3.15 mmol) was added to a slurry of 1.0 g mesoporous silica microparticles in 150 mL reagent ethanol and was heated to 75 degrees Celsius for 7 hours. After cooling to room temperature and the slurry was filtered and the particles were washed with deionized water followed by ethanol and then dried in an oven at 100 degrees Celsius for 20 hours to afford 11-azidoundecyl functionalized microparticles.

### Example 2(o): 3-Azidopropyl Functionalized Microparticles

(3-azidopropyl)trimethoxysilane (1.0 g, 4.87 mmol) was added to a slurry of 1.0 g mesoporous silica microparticles in 150 mL reagent ethanol and was heated to 75 degrees Celsius for 7 hours. After cooling to room temperature and the slurry was filtered and the particles were washed with deionized water followed by ethanol and then dried in an oven at 100 degrees Celsius for 20 hours to afford 3-azidopropyl functionalized microparticles.

### Example 2(p): 3,3,4,4,5,5,6,6,7,7,8,8,8-Tridecafluorooctyl Functionalized Microparticles

Triethoxy(3,3,4,4,5,5,6,6,7,7,8,8,8-tridecafluorooctyl)silane (2.499 mL, 6.51 mmol) was added to a slurry of 1.0 g mesoporous silica microparticles in 150 mL reagent ethanol and was heated to 75 degrees Celsius for 7 hours. After cooling to room temperature and the slurry was filtered and the particles were washed with deionized water followed by ethanol and then dried in an oven at 100 degrees Celsius for 20 hours to afford 3,3,4,4,5,5,6,6,7,7,8,8,8-tridecafluorooctyl functionalized microparticles.

### Example B. Testing MSR Surface Modifications for Virus Binding

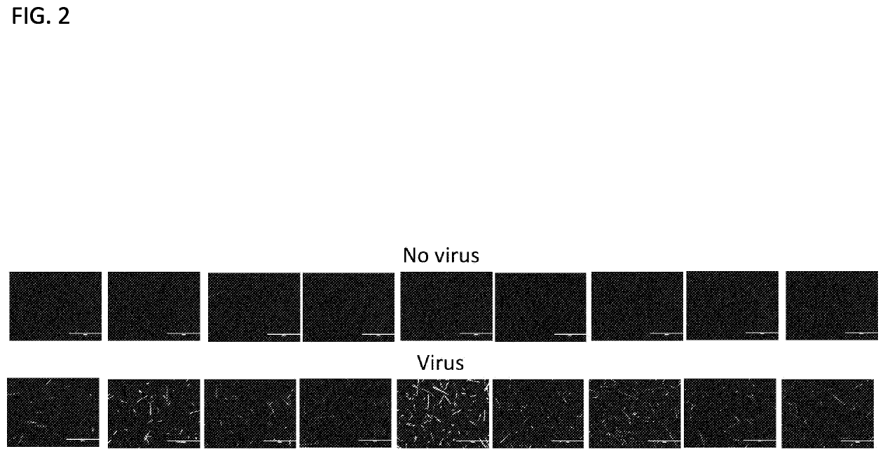

To test the binding of lentivirus to MSPs, a variety of MSPs was prepared with varying surface chemistries (FIG. 1). Dry MSR batches were resuspended at 10 mg/ml in ice-cold Tris-NaCl-EDTA buffer pH 7.5 (NTE buffer). A stock solution of green fluorescent protein (GFP) expressing lentivirus (FCT067, Kerafast) was diluted in ice-cold NTE buffer to a titer of 3×10⁶/ml. The MSR suspension and the diluted virus were combined at ratios of 1:1 vol/vol and incubated on ice for 30 minutes. Control particles were incubated 1:1 vol/vol with NTE buffer without virus. Following the incubation, samples were washed once with 1% bovine serum albumin (BSA) in phosphate buffered saline (PBS) at 4° C., and then with PBS at 4° C. Samples were then fixed with 4.2% paraformaldehyde in PBS. Samples were stained with an antibody against the viral envelope (anti-VSV-G from Kerafast, 8G5F11; 1:50 dilution) followed by an anti-mouse IgG labeled with Dylight-488 (Invitrogen). Samples were washed twice with PBS and imaged using an Evos fluorescent microscope equipped with a GFP LED light cube (FIG. 2). Imaging showed no detectable binding of the staining reagents to MSRs bearing no virus. Virus-conjugated rods show varying levels of quantitative binding, with the trimethylammonium and amine functionalities showing maximum binding.

### Example C. In Vitro Assay for T Cell Transduction with GFP Lentivirus Using MSR

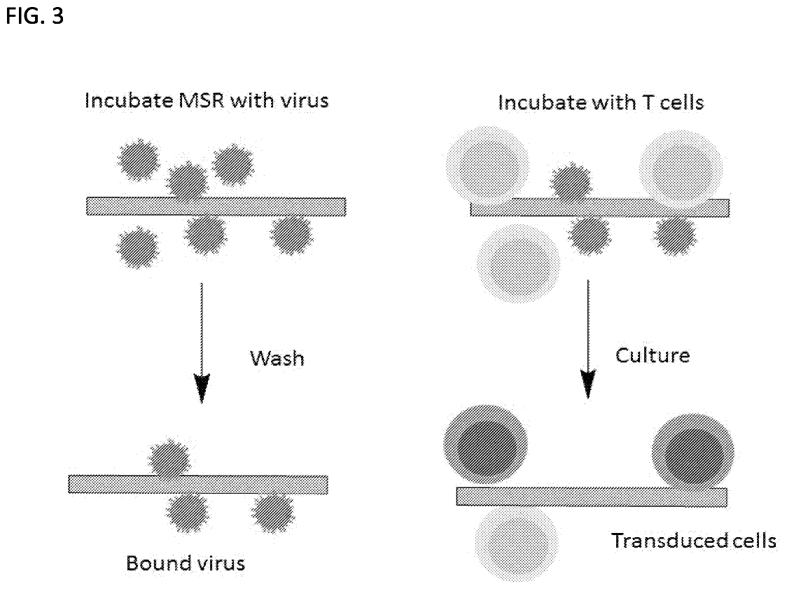

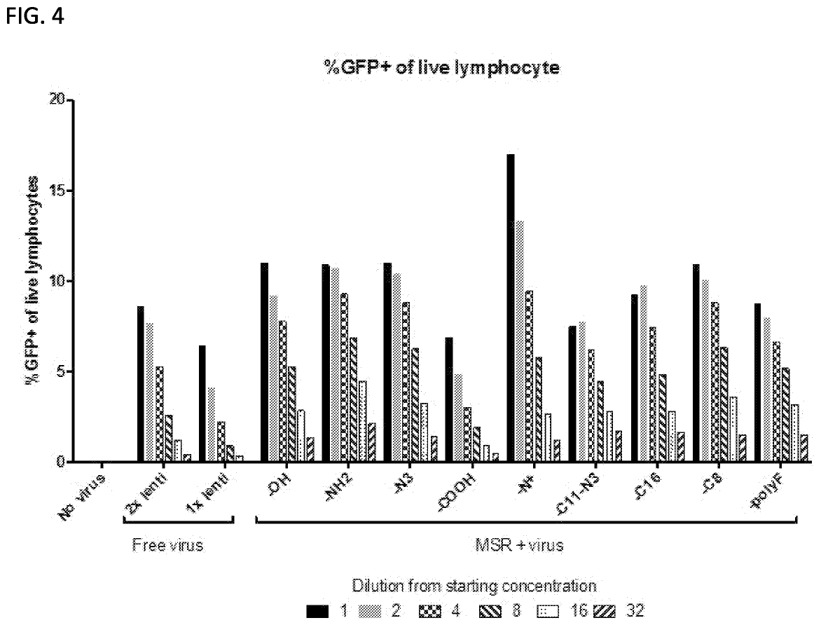

A schematic representation of the use of MSRs for virus transduction of T cells is shown in FIG. 3. Naïve human T cells were stimulated with Dynabead T cell activator beads at a 3:1 bead:cell ratio for two days. Beads were removed using a magnet, and cells were transferred to fresh culture medium. Virus-conjugated MSRs were prepared as noted above and resuspended at 80 μg/ml in cell culture medium. Serial dilutions of this were made as indicated in FIG. 3. This suspension was combined 1:1 with T cells 5×10⁵/ml and incubated for four days. GFP expression was assessed in live, singlet, cells in culture to assess the transduction efficiency. Results (FIG. 4) indicate that the transduction of MSR-conjugated virus occurred at greater levels than virus given in culture media only. The trimethylammonium functionalized MSRs provided the highest level of transduction.

### Example D. Interaction of T Cells with MSRs Presenting CD3/CD28 Agonistic Antibodies, EGFRvIII Peptides, or BCMA Protein

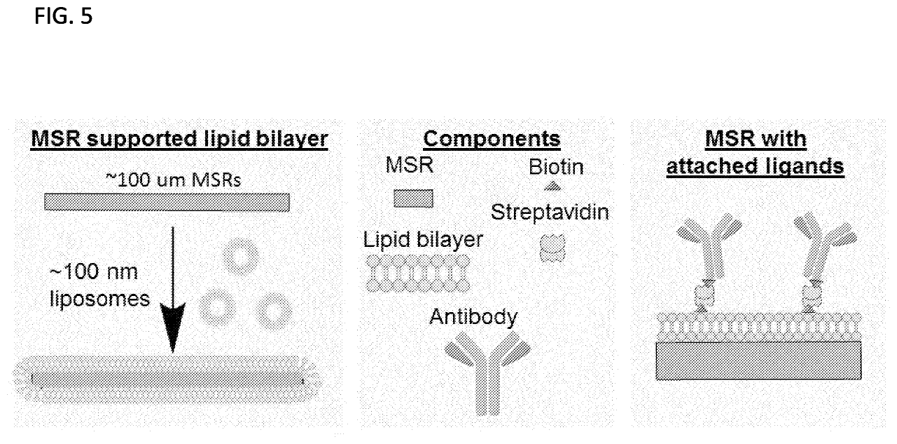

MSRs with surface-immobilized ligands were prepared as described in Cheung, A. S., et al., Scaffolds that mimic antigen-presenting cells enable ex vivo expansion of primary T cells. Nature Biotechnology, 36(2), 160-169. A schematic of this process is shown in FIG. 5.

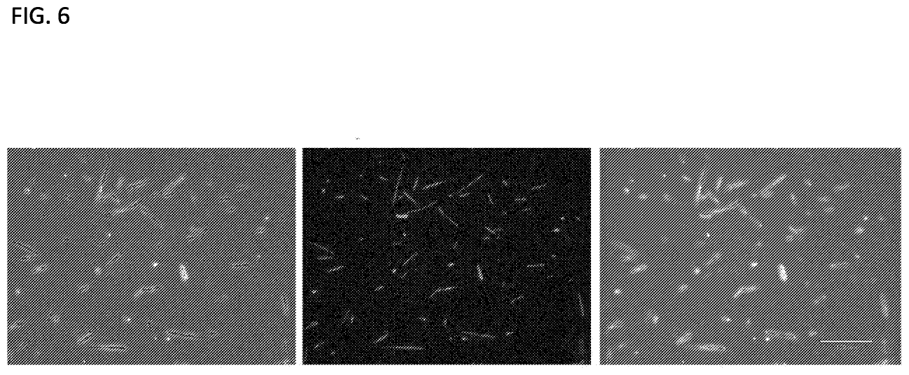

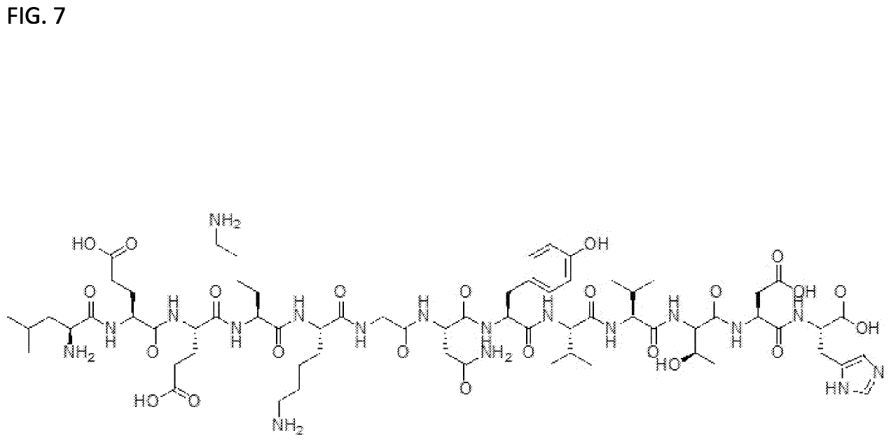

Briefly, liposomes primarily composed of POPC with 1 mol % PE-biotin were formed using a thin film rehydration method and extrusion through a 100 nm polycarbonate membrane. Hydroxyl functionalized MSRs were incubated with the liposomes to allow the formation of a supported lipid bilayer on the MSR surface (FIG. 6). To functionalize the MSRs with CD3 and CD28 agonistic antibodies, MSRs were washed several times with PBS, incubated with streptavidin, and then tethered with biotinylated CD3 and CD28 antibodies. For MSR-immobilization of EGFRvIII CAR-binding peptides, a biotinylated EGFRvIII CAR-binding peptide was used (FIG. 7). For BCMA CART stimulation, recombinant BCMAFc protein was biotinylated using biotin-NHS and similarly coupled to the MSR surface.

After incubation with the desired ligands, MSRs were washed several times with PBS and resuspended in culture medium at various concentrations and incubated with T cells. T cell proliferation was read out using CFSE labeling of the T cells and assessing dye dilution by flow cytometry. Cytokine production was assessed using a multiplex cytokine analysis method (Mesoscale Delivery V-Plex).

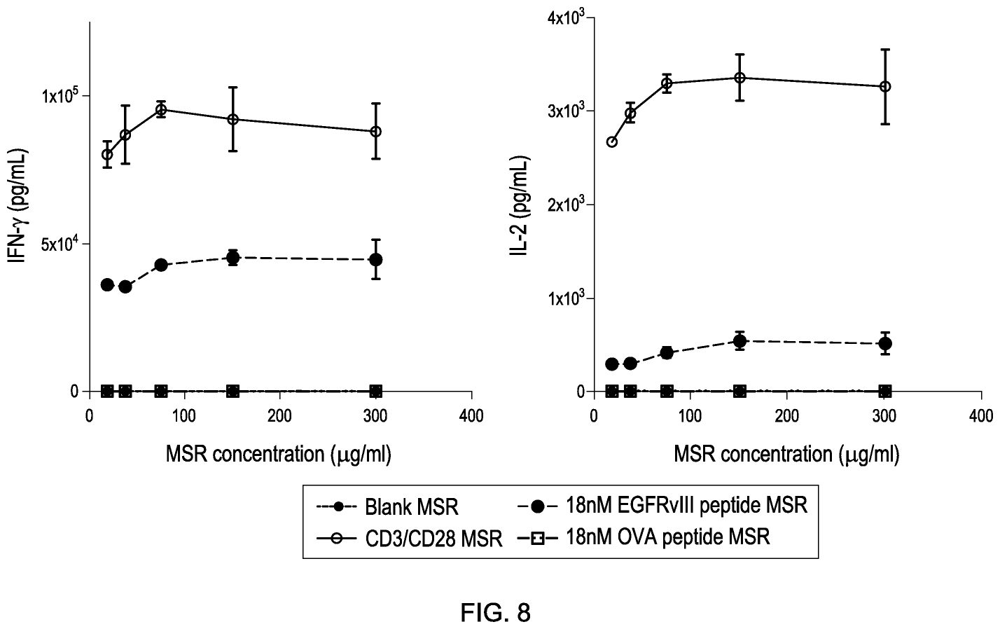

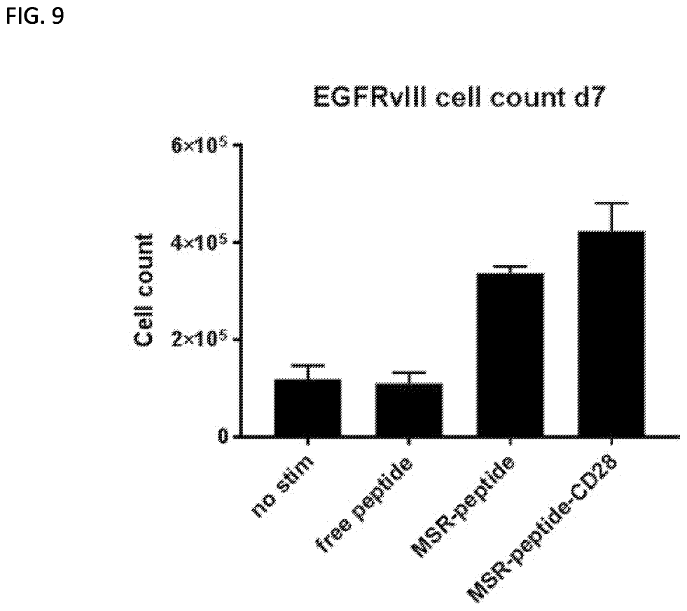

EGFRvIII CARTs produced interferon gamma and IL-2 in response to EGFRvIII CAR-binding peptide bound to the surface of the MSRs, while free EGFRvIII CAR-binding peptide in solution, a non-stimulating peptide (OVA) presented on the MSRs, or undecorated MSRs gave no response from the CARTs (FIG. 8). In another experiment, the proliferation of EGFRvIII CARTs were monitored in response to various stimuli using cell counting (FIG. 9).

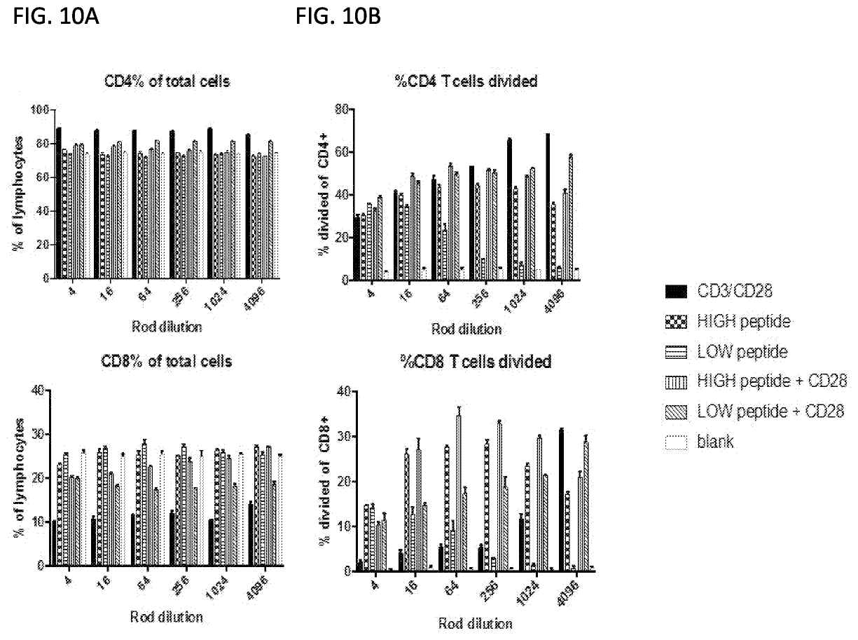

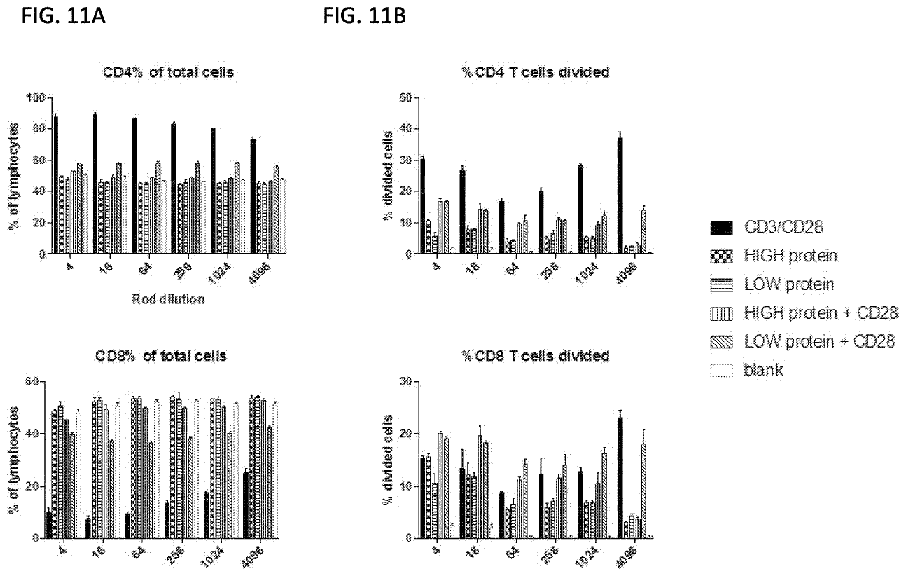

To further analyze the phenotype expansion of different T cell subsets, proliferation of EGFRvIII CARTs was assessed using flow cytometry. CARTs were stained with CFSE and monitored for dye dilution to indicate proliferation by flow cytometry (FIG. 10). Similar experiments were conducted using MSRs functionalized with BCMAFc protein antigen present on the MSR surface (FIG. 11).

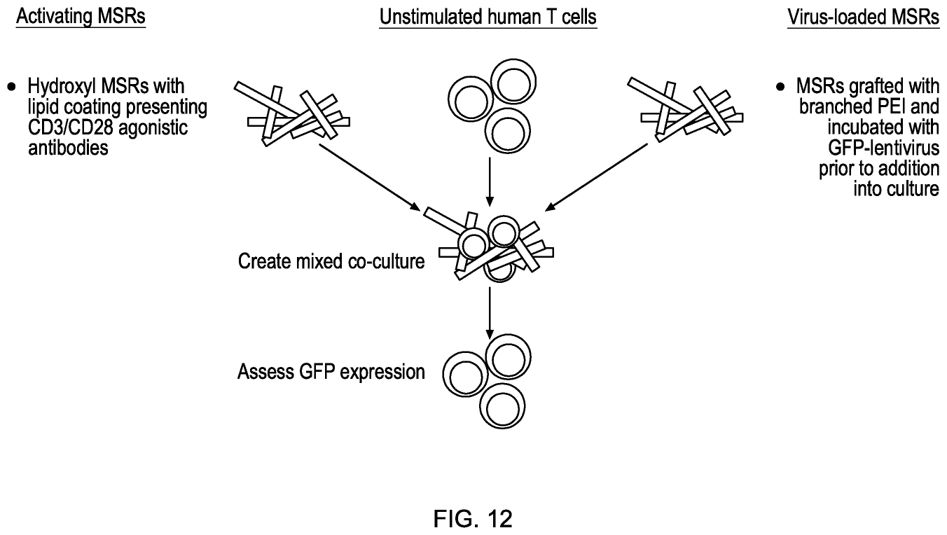

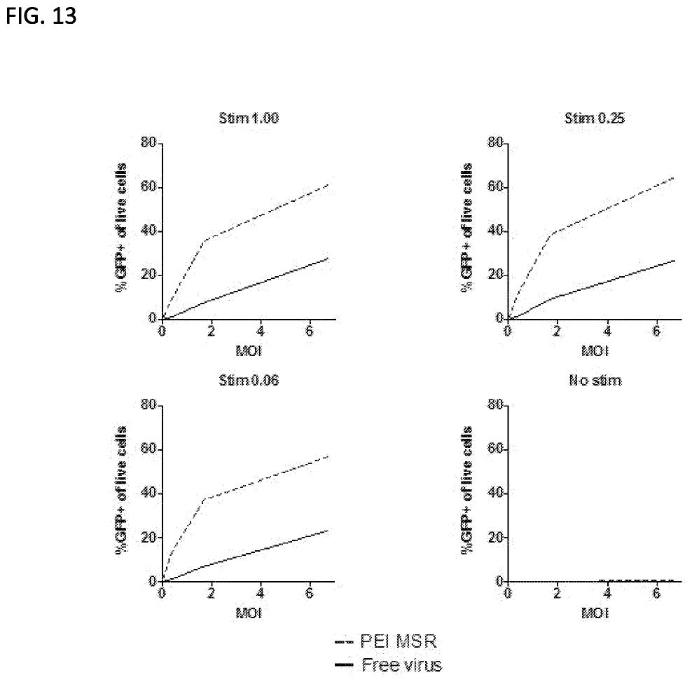

To test the simultaneous stimulation and transduction of T cells with virus using two types of MSRs (MSRs bearing stimulatory cues, and MSRs mixed with lentivirus), the experimental schema shown in FIG. 12 was used. One population of MSRs were coated with a lipid bilayer and grafted with anti-CD3/CD28 antibodies as described above. A second population of MSRs were incubated with lentivirus. Results shown in FIG. 13 indicated a superior transduction level when T cells were stimulated with anti-CD3/CD28 agonistic antibodies and were exposed to virus that was incubated with PEI-MSRs compared to free virus in solution.

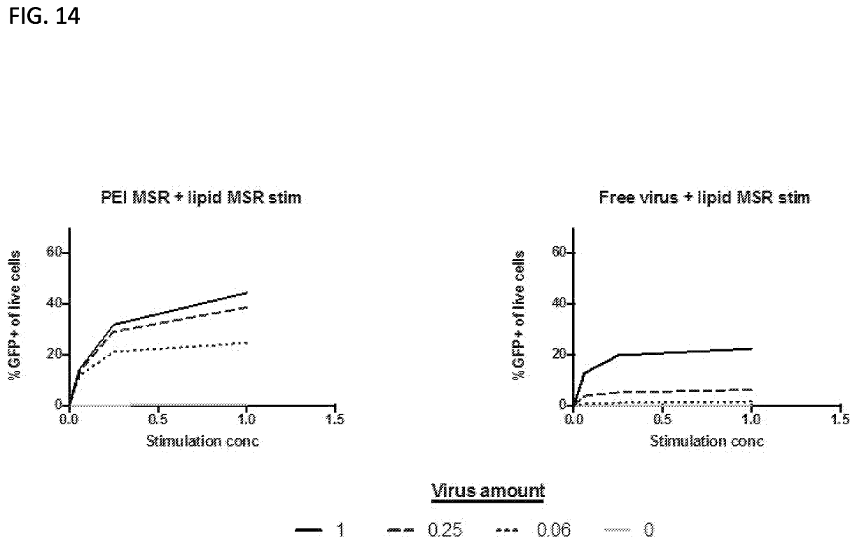

To test the simultaneous stimulation and transduction of T cells with both cues on the same population of MSRs, T cells were exposed to either (1) anti-CD3/CD28 agonistic antibody-bearing lipid-coated stimulating MSRs, and virus in media, (2) anti-CD3/CD28 agonistic antibody-bearing lipid-coated stimulating MSRs, and PEI-MSRs pre-incubated with virus, or (3) PEI MSRS adsorbed with anti-CD3/CD28 agonistic antibodies, and then incubated with virus. After three days of culture, T cells were assessed for transduction efficiency. FIG. 14 shows the effect of stimulatory MSR concentration on the MSRs of conditions (1) and (2) above at various amounts of virus. As shown in the FIG. 14, overall transduction is enhanced under condition (2) where PEI-MSRs are incubated with virus.

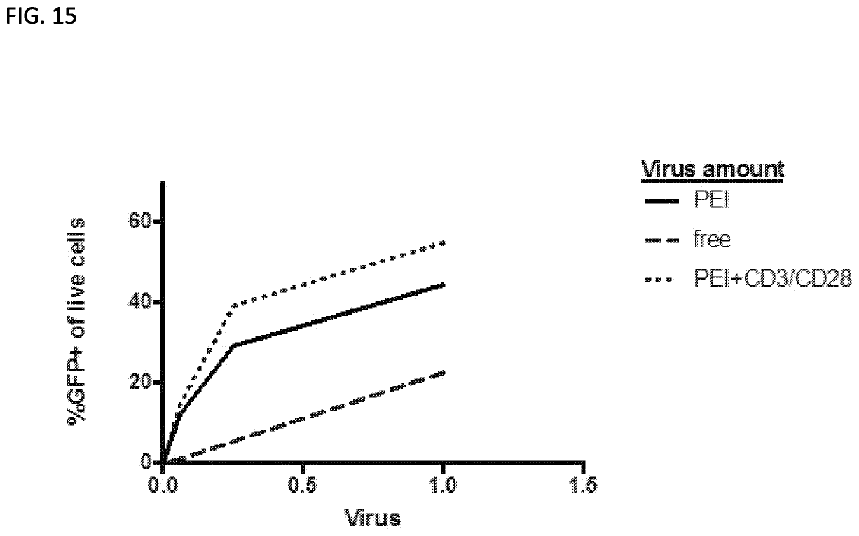

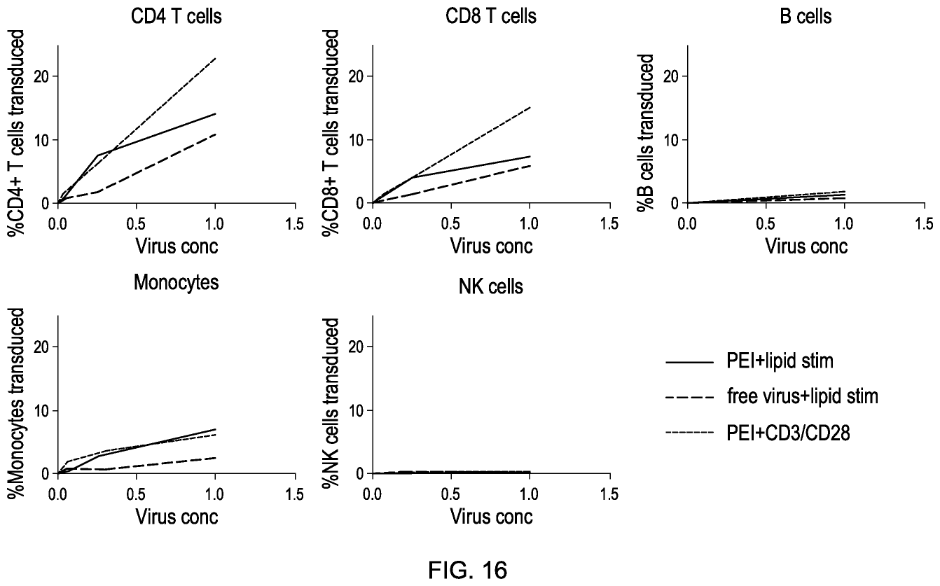

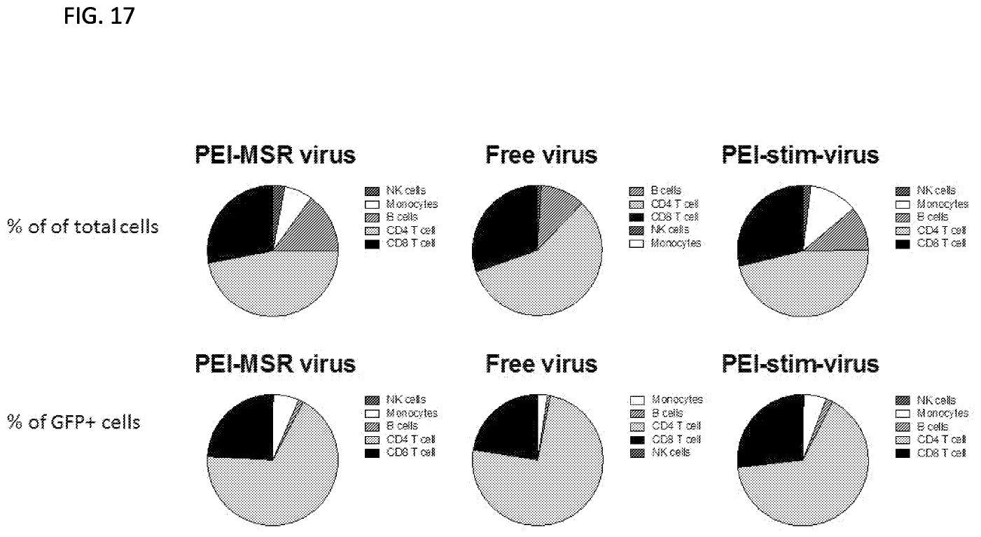

FIG. 15 compares all three conditions, where conditions (1) and (2) are at the highest concentration of stimulatory MSRs. As seen in FIG. 15, condition (3) where stimulatory cues are bound to the PEI-MSRs produces the highest relative transduction efficiency. The same formulations were used to study MSR-mediated transduction with human peripheral blood mononuclear cells (PBMCs). In FIG. 16, the transduction in various cell populations as a function of virus concentration is shown at the highest level of stimulation for conditions (1) and (2). FIG. 17 shows the proportion of each cell population present in the total GFP+ transduced cell fraction, and in the total cell population collected at the highest level of stimulation for conditions (1) and (2).

### Example E. In Vivo Study of MSR Induced T Cell Transduction

A composition of mesoporous silica particle conjugated to viral vectors is injected under the skin of mice. Approximately 5-7 days later, MSRs adsorbed with a virus encoding an anti-mouse CD19 CAR is injected at this site. The depletion of CD19+B cells in the blood of mice will be monitored as an indication that anti-CD19 CARTs have been generated. The presence of these CARTs is confirmed in the blood and bone marrow. Detailed histological assessment of the injection site as well as draining lymph nodes, the spleen, and liver, using in situ hybridization for the CAR transgene is conducted to assess the leakage of the virus to unwanted sites.

### Example F. Drug Loading onto Mesoporous Silica Microparticles

A variety of drugs may be loaded onto the mesoporous silica microparticles.

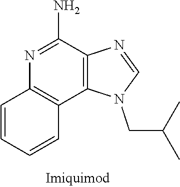 [SMILES: CC(C)Cn1cnc2c(N)nc3ccccc3c21]

### 1. Example 1: Loading of TLR7 Agonist onto Mesoporous Silica Microparticles

A solution of Imiquimod, in chloroform is added to a slurry of 100 mg silica microparticles in 2.0 mL chloroform (a concentration of 100-500 μg of imiquimod per 10 mg of mesoporous silica particles) and shake at 500 rpm at 40 degrees Celsius for 72 hours. The MSPs are centrifuged at 1000 rpm for 3 min and the remaining solution is removed. The MSPs are washed with 2.0 mL of chloroform followed by centrifugation and removal of the supernatant. The wash steps are repeated with ethanol to remove excess and unabsorbed imiquimod. The final microparticles are slurried in water and lyophilized.

### 2. Example 2: In Vitro Drug Release from Mesoporous Silica Particles

10.0 mg (or the equivalent of 300 μg of drug-loaded material) drug-loaded MSPs are suspended in 1.0 mL of pH 7.4 (0.0067M) phosphate buffer and left at 37 degrees Celsius. Samples are collected at 1 h, 3 h, 6 h, 24 h, 2 days, and 5 days; analysis of these samples is performed by UPLC and plotted to a standard analytical curve. Supernatant is removed and replaced with fresh buffer at each timepoint.

The foregoing written specification is considered to be sufficient to enable one skilled in the art to practice the invention. The present invention is not to be limited in scope by the constructs deposited, since the deposited embodiments are intended to illustrate only certain aspects of the invention and any constructs that are functionally equivalent are within the scope of this invention. The deposit of material herein does not constitute an admission that the written description herein contained is inadequate to enable the practice of any aspect of the invention, including the best mode thereof, nor is it to be construed as limiting the scope of the claims. Indeed, various modifications of the invention in addition to those shown and described herein will become apparent to those skilled in the art from the foregoing description and fall within the scope of the appended claims.

It is understood that the application of the teachings of the present invention to a specific problem or situation will be within the capabilities of one having ordinary skill in the art in light of the teachings contained herein.

The disclosures of each and every citation in the specification are expressly incorporated herein by reference.

### Example G. In Vivo Study of MSR Induced CAR-T Generation

Mice engrafted with human T cells and B cells (human CD34+ stem cell humanized mice or human peripheral blood mononuclear cell-injected mice) are established using known methods. A composition of mesoporous silica particle conjugated to CAR19 lentivirus is injected under the skin of mice to transduce T cells. The presence of CAR19-expressing T cells (using anti-CAR19 idiotype antibody staining) and the depletion of CD19+B cells in the blood of mice treated with MSR-CAR19 lentivirus conjugates is monitored using flow cytometry on serial blood collection samples (day 0 pre-injection and twice weekly between day 1 to day 21 following MSR-virus injection) and compared to control mice injected with MSR-GFP lentivirus as an indication that anti-CD19 CARTs have been generated and are functional in killing their target. The concentration of human interferon-gamma and tumor necrosis factor alpha is determined from the same blood samples as a second biomarker for CD19 CAR T cell generation and activation. Detailed histological assessment of the injection site as well as lymph nodes, bone marrow, the spleen, and liver using in situ hybridization for the CAR transgene is conducted to assess the leakage of the virus to unwanted sites and study trafficking of the generated CAR19 T cells to these sites.

In another experiment, human T and B cell-containing mice are intravenously injected with a CD19-expressing Nalm6 leukemia tumor that expresses a luciferase reporter gene. Cohorts of mice are injected under the skin with a single injection of a composition of mesoporous silica particles conjugated to CAR19 or GFP lentivirus from 7 days before to 7 days following tumor injection to transduce T cells. The Nalm6 tumor burden is monitored by luciferase signal on IVIS imaging to study anti-tumor efficacy of the generated anti-CD19 CARTs. The presence of CAR19-expressing T cells and the depletion of CD19+B cells in the blood of mice treated with MSR-CAR19 lentivirus conjugates is monitored on serial blood collection samples (day 0 pre-injection and twice weekly between day 1 to day 21 following MSR-virus injection) and compared to control mice injected with MSR-GFP lentivirus. The concentration of human interferon-gamma and tumor necrosis factor alpha is determined from the same blood samples as a second biomarker for CD19 CAR T cell generation and activation.

These studies are repeated with MSR-lentivurs conjugates for other cancer/tumor targets, including but not limited to BCMA, CD20, CD22, CD123, EGFRvIII, CLL-1, and combinations thereof (with each other and/or CD19).

### Example H: Functional Analysis of Compositions Comprising a Cryogel Comprising VEGF-C and MSRS with Conjugated Viral Vector and Absorbed Cell Activation Agent

### Example 1: Generation of VEGF-C

HEK Production

DNA was synthesized at GeneArt (Regensburg, Germany) and cloned into a mammalian expression vector using restriction enzyme-ligation based cloning techniques. The resulting plasmid was transfected into HEK293T cells. For transient expression of proteins, vectors for wild-type or engineered variants were transfected into suspension-adapted HEK293T cells using Polyethylenimine (PEI; Cat #24765 Polysciences, Inc.). The recombinant expression vectors were then introduced into the host cells and the construct produced by further culturing of the cells for a period of 7 days to allow for secretion into the culture medium.

The produced constructs were then purified from cell-free supernatant, using immobilized metal ion affinity chromatography (IMAC).

His-tagged proteins were captured by IMAC. The resin was washed before the protein was eluted.

Finally, eluted fractions were polished by using size exclusion chromatography (allowing separation of aggregates, monomers and dimers. Purification analysis was done using SDS-PAGE and aggregation content was determined using analytical size exclusion chromatography method.

TABLE 21
Production yield of VEGF-C variants in HEK293T cell line

| | Physical | Final yield after IMAC |
|---|---|---|
| Variant | state | and SEC (ug/L) |
| 1 | Dimer | 117 |
| 2 | Dimer | 405 |
| | Monomer | 714 |
| 7 | Dimer | 1120 |
| | Monomer | 623 |
| 8 | Dimer | 1190 |
| 9 | Dimer | 869 |
| | Monomer | 125 |

CHO Production

A CHO MaKO manufacturing expression system was used to produce VEGF-C variant 8. The gene encoding the target protein was introduced into an expression cassette driven by a CMV promoter in a plasmid expression vector. The vector was transfected in triplicate into CHO MaKO cells. For each transfection, 0.5 μg of plasmid were transfected into viable cells in medium. The transfected cells were seeded into cell culture medium with low concentration of folic acid in shake flasks. Cells were grown in a humidified shaker incubator. On day 3 post transfection, selection for stable transfectants was started. The cells went into a selection crisis and recovered within 21 days. Then vials of the selected stable pools were frozen.

For production of the VEGF-C variant 8 a fed-batch approach was used. A vial of the frozen cells was thawed. After recovery from thawing, cells were seeded into production cell culture medium in shake flasks. Cultures were grown in a humidified shaker incubator. Growth temperature was decreased on day 5 after seeding the culture. Feed solutions were added on day 3, 4, 5, 6, 7 and 10 after seeding. The culture was harvested on day 11 after seeding. Cells were separated from the cell culture medium by centrifugation and sterile filtering. The target protein was purified from the clarified cell culture supernatant and characterized as above.

TABLE 22
Production yield of VEGF-C variant 8 in CHO MaKO cell line

| | Physical | Final yield after IMAC |
|---|---|---|
| Variant | state | and SEC (mg/L) |
| 8 | Dimer | 17.5 |

### Example 2: Generation of Cryogel

Alginate conjugations were formulated based on a previously described protocol (Koshy et al. 2018).

Norbornene Alinate (Alg-Nb)

One gram of Pronova UP MVG alginate was dissolved in 100 ml of 0.1 M 2-(N-morpholino)ethanesulfonic acid (MES) buffer overnight at room temperature under constant stirring. 280 μl of 5-norbornene-2-methylamine (norbornene) was then added to the alginate solution. 1464 mg of 1-ethyl-3-(3-dimethylaminopropyl)-carbodiimide hydrochloride (EDC) and 1085 mg of N-hydroxysuccinimide (NHS) were separately dissolved in 20 ml of MES buffer and added to the alginate-norbornene solution and allowed to react for 24 hours at room temperature. After 24 hours the solution was dialyzed in sequential 5 L salt baths (7, 6, 5, 4, 3, 2, 1, 0, 0, 0 g/L NaCl) for 3 hours at each concentration. Solution was then filtered twice (0.22m vacuum) and frozen at −80 C overnight. Frozen solutions were then lyophilized for 5 days and stored at −20 C until use in experiments.

Tetrazine Alinate (Alg-Tz)

One gram of Pronova UP MVG alginate was dissolved in 0.1 M 2-(N-morpholino)ethanesulfonic acid (MES) buffer overnight at room temperature under constant stirring. 126 mg of (4-(1,2,4,5-tetrazin-3-yl)phenyl methanamine hydrochloride (tetrazine) was then added to the alginate solution. 1464 mg of 1-ethyl-3-(3-dimethylaminopropyl)-carbodiimide hydrochloride (EDC) and 1085 mg of N-hydroxysuccinimide (NHS) were separately dissolved in 20 ml of MES buffer and added to the alginate-tetrazine solution and allowed to react for 24 hours at room temperature. After 24 hours, solution was quenched with 311 mg of hydroxylamine for 30 min and centrifuged at maximum RPM for 10 minutes. Solution was then filtered (0.22 uM filter) and dialyzed in sequential 5 L salt baths (7, 6, 5, 4, 3, 2, 1, 0, 0, 0 g/L NaCl) for 3 hours at each concentration. Solution was then filtered twice (0.22 uM filter) and frozen at −80 C overnight. Frozen solutions were then lyophilized for 5 days and stored at −20 C until use in experiments.

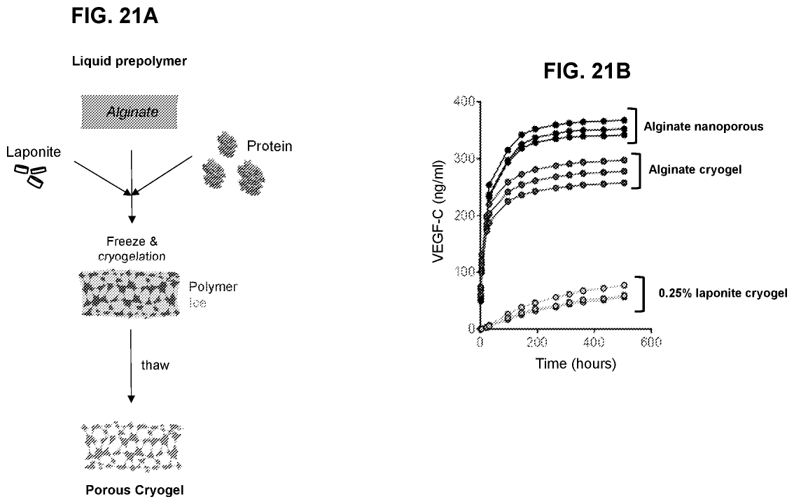

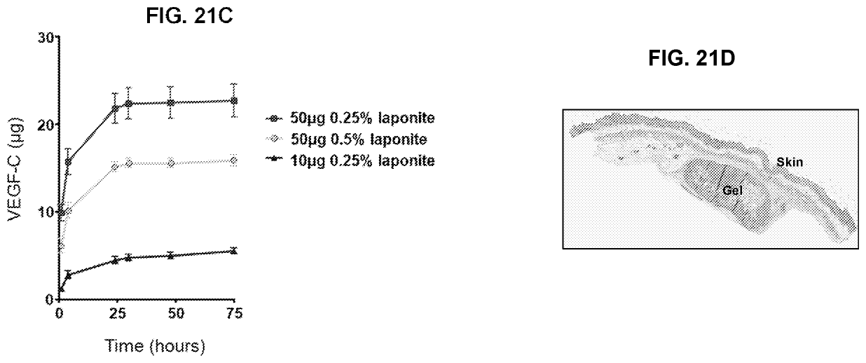

Cryogel Formulation (FIG. 21A)

Alg-Tz and Alg-Nb were dissolved in DIH₂O at 20 mg/ml with a Thermomixer (37 C, 2000 rpm) for 1 hour. Separately, laponite was dissolved for 1 hour at 5 mg/ml concentration in DI-H₂O under constant mixing at room temperature. Solutions were then filtered (0.22 uM filter) under sterile conditions. VEGF-C(3 mg/ml stock) and laponite (Laponite XLG) (5 mg/ml stock) solutions were incubated together at room temperature for 1 hour. Alg-Tz solution and Alg-Nb solutions were then added in a 1:1 ratio and diluted to a final concentration of 10 mg/ml (0.25 mg/ml final concentration of laponite, 10 μg VEGF-C/gel). 50 ul of mixture was immediately pipetted into PEEK molds and frozen at −20 C overnight. Prior to injection, gels were thawed at room temperature in the molds and then placed into 16ga syringe needles. Needles with gels were then placed on 1 ml syringes with 100 ul of sterile PBS.

### Example 3: VEGF-C Release Analysis

In Vitro VEGF-C Release Assay from Alginate Cryogels (FIG. 21)

VEGF-C cryogels (10 μg protein+0.25 mg/ml laponite) were incubated at 37 C in 1 ml of release buffer (1% BSA solution in PBS). Release buffer was completely removed and replaced at various timepoints throughout the experiment. Samples of release buffer were stored at −80 C until thawed for use in a VEGF-C ELISA.

### Example 4: MSR Synthesis

Poly(ethylene glycol)-block-poly(propylene glycol)-block-poly(ethylene glycol) avg Mn ˜5,800 (Pluronic P-123, 80.0 g, 487 mmol; Sigma) surfactant was dissolved in 3 L of 1.6M HCl at room temperature, heated to 40 C in a 5 L jacketed flask, and mechanically stirred via and overhead stirrer at a rate of 0-600 rpm. Tetraethyl orthosilicate (TEOS, 184 mL, 826 mmol; Sigma) was added in one portion over <5 min and was heated at 40 C with maintained stirring for at least 2 hours but most commonly 20 hours. The resulting slurry was heated to 80-130 C for 6-72 hours for hydrothermal treatment before being cooled to room temperature. The slurry was filtered in a Buchner funnel and was washed with deionized water followed by ethanol and air dried at room temperature. The resulting silica material was calcined in a furnace with a slow ramp temperature from room temperature to 550 C over 8 hours and then maintaining at 550 C for another 8 hours before cooling to room temperature to afford 47 g of mesoporous silica particles.

Changes in the stir rate may have changes in the microparticle aspect ratio. Varying the conditions of the hydrothermal temperature and duration are common pore size controllers for mesoporous materials.

Final mesoporous materials were characterized by light microscopy, Malvern Morphologi G3, scanning electron microscopy (SEM), thermal gravimetric analysis (TGA).

N,N,N-Trimethylpropan-1-Ammonium Functionalized Microparticles

Trimethoxysilylpropyltrimethylammonium chloride (3.61 mL, 6.51 mmol; 50% solution in methanol) was added to a slurry of 1.0 g mesoporous silica microparticles in 150 mL reagent ethanol and was heated to 75 degrees Celsius for 7 hours. After cooling to room temperature and the slurry was filtered and the particles were washed with deionized water followed by ethanol and then dried in an oven at 100 degrees Celsius for 20 hours to afford N,N,N-trimethylpropan-1-ammonium functionalized microparticles, referred to herein as “trimethylammonium MSRs”.

MSR Size Reduction

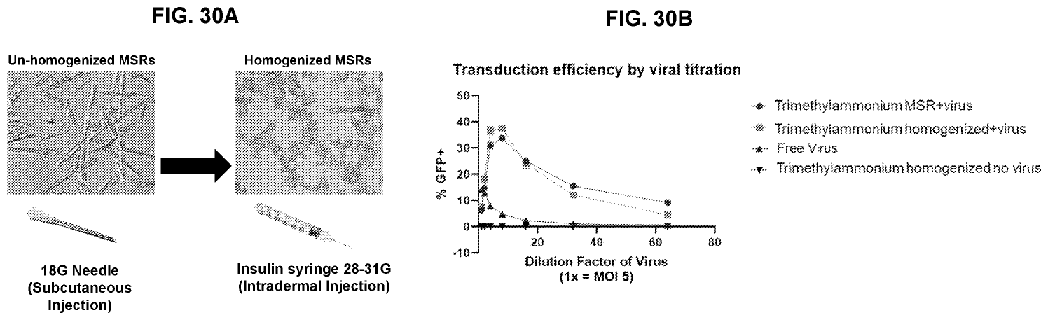

Stock MSR variants were roughly 100-200 μm in length after synthesis. To improve injectability through a 28.5 ga insulin syringe, dry MSRs were homogenized in a MP FastPrep-24 5G bead beating grinder and lysis system for 80 seconds (FIG. 30). MSRs were then autoclaved and stored at room temperature until used in experiments.

Loading of Lentivirus onto MSRs and Characterization of Complex

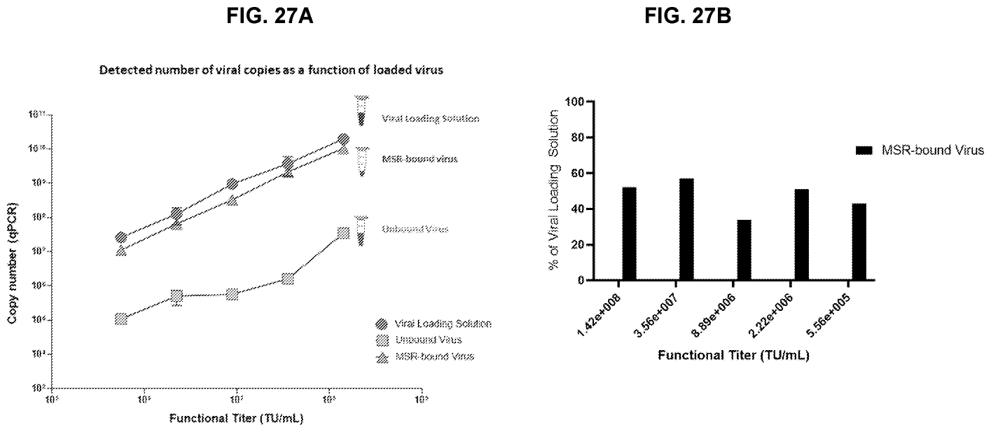

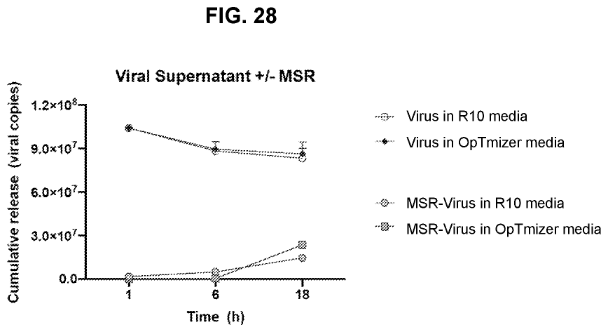

Homogenized MSR batches were resuspended at 10 mg/ml in ice-cold Tris-NaCl-EDTA buffer pH 7.5 (NTE buffer). The desired lentivirus stock was diluted in ice-cold NTE buffer to the desired total amount of transducing units (TU) for loading. The MSR suspension and the diluted virus were combined at ratios of 1:1 vol/vol and incubated on ice for 30 minutes. At least two wash steps in excess DPBS were performed to remove excess virus prior to functional in vitro tests, or in vivo use. For loading and retention studies (FIGS. 27-28), a 2.5 mg/ml MSR suspension was mixed 1:1 with of a solution containing the indicated amount of virus in the figures and incubated for 30 minutes on ice. Virus amounts in loading solutions, bound to the MSRs, and released from the MSRs was quantified using a commercially available kit. For release studies, the MSR-lentivirus complex was incubated in the media at 37 C, and the supernatant was collected at the indicated timepoints for qPCR analysis.

MSR Loading with Virus and STARTERS for In Vivo Use

Construct 2 (Table 20, FIG. 48A-48B) and trimethylammonium MSRs were co-incubated to allow adsorption of the cell activation agent onto the MSR surface. Construct 2 was added to 1 an 8 mg/ml trimethylammonium MSR suspension and incubated for 1 hour at 4 C. Loaded MSRs were washed three times and resuspended in DPBS to a final concentration of 15 mg/ml MSRs.

NTE buffer solution containing CD19 CAR encoding (also referred to as CAR19) lentivirus was mixed with a 10 mg/ml trimethylammonium MSR suspension and incubated for 30 minutes at 4 C. MSRs were washed twice and resuspended in DPBS to a final concentration of 15 mg/ml MSRs.

Finally, MSRs were vigorously pipetted up and down, back loaded into insulin syringes, and immediately injected intradermally into mice.

### Example 5: Transduction of Cells and Functional Testing

In Vitro T Cell Transduction

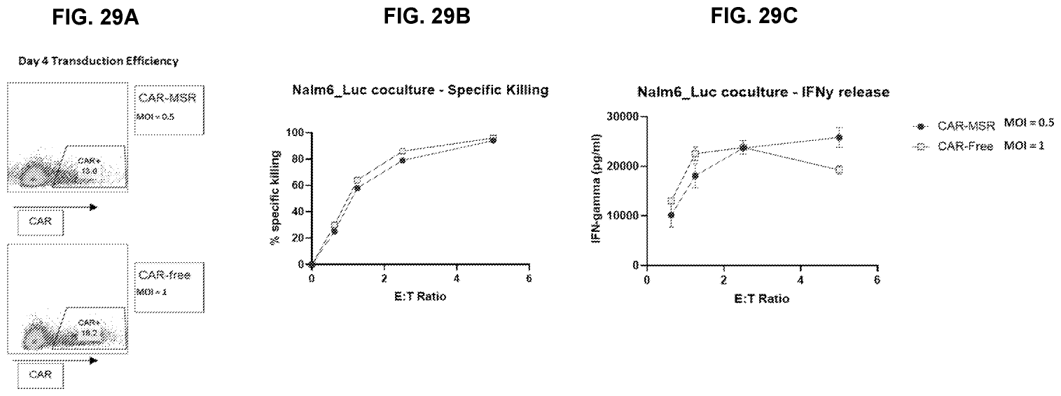

Human T cells were isolated from leukopaks using the Miltenyi Human Pan T cell isolation kit and frozen prior to use. Cells were thawed and plated in complete OpTmizer medium in the presence of the format 4 construct (Table 20, FIGS. 48A-48B). CD19 CAR encoding (also referred to as CAR19) virus, either as free virus or MSR-virus complexes were added into the culture, followed by one day of incubation. Cells were washed and plated for a further three days prior to use in characterization and functional tests (FIG. 29-30).

CAR T Cell Characterization and Functional Testing (FIG. 29)

CAR T cells were analyzed for expression of the CAR receptor by staining with a CD19 CAR anti-idiotype antibody conjugated to PE and analysis by flow cytometry. Nalm6 (RRID: CVCL_0092) is a human acute lymphoblastic leukemia (ALL) cell line. Cells were grown in RPMI medium containing 10% fetal bovine serum and both grow in suspension. Cells were modified to express luciferase (Nalm6-Luc), so that their presence in co-cultures could be assessed by luciferase signal. Nalm6-Luc cells were co-cultured at various cellular ratios with CD19-CARTs produced using free virus of MSR-virus complexes (FIG. 29). The luciferase signal at the end of a 1-day coculture was used to calculate the percentage of input Nalm6 that had been killed by the CARTs. Interferon-gamma levels in the supernatant at the end of the co-culture were quantified using a commercially available kit.

### Example 6: Analysis of Results

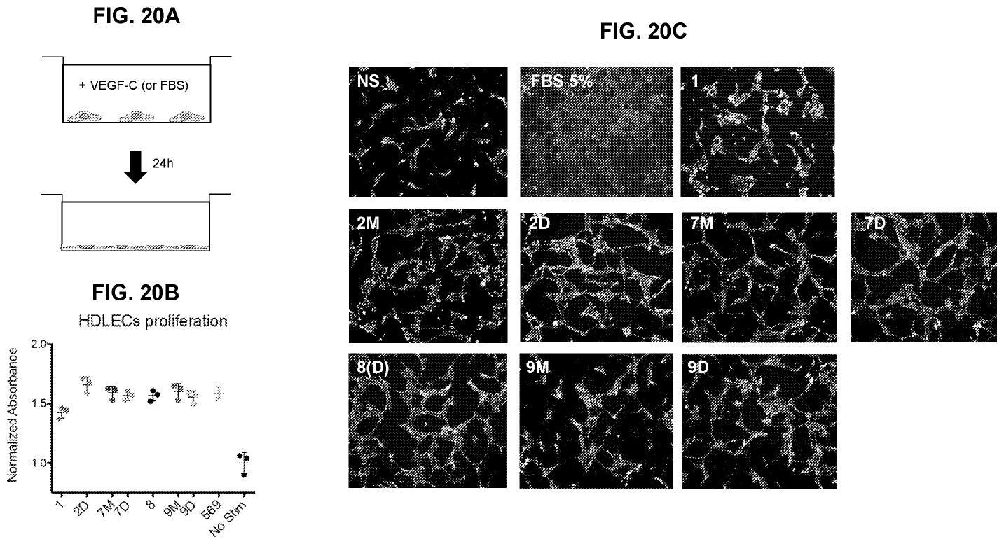

Tube Assay (FIG. 20)

Human Dermal Lymphatic Endothelial Cells (HDLECs) were seeded (p0) and passaged at 75% confluence until p4 or enough cells were obtained for experiments. One ml was then aliquoted into Eppendorf tubes, treated with 600 μg of VEGF-C protein, and vortexed. Each treatment was added to warmed media in a 6 well plate and incubated overnight at 37 C (5% CO₂). For imaging analysis cells were washed and then fixed with 3.7% formalin-0.05% Triton X-100 solution (fixing buffer), then washed and coated with 0.1% Triton X-100 solution (permeabilization buffer). After washing twice with PBS cells were coated with 0.05% Triton X-100 in 1% BSA solution (blocking buffer). DAPI and phalloidin staining was performed according to known protocols. After staining cells were washed twice with PBS and imaged.

Proliferation Assay (FIG. 20)

WST-8 media was prepared by diluting WST-8 solution 1:10 into MV media. Culture media was removed, and cells were incubated in WST-8 media. Following incubation, media was removed in triplicate from each well and added to a 96 well plate. Absorbance of each well at 450 nm was read using a spectrophotometer.

Tissue Processing after In Vivo Experiments

Tissues were harvested from mice, weighted, and cut into very fine pieces with scissors. Samples were then enzymatically digested under constant stirring in of digestion media containing collagenase 4 and DNAse 1. Samples were then pipetted up and down. Following pipetting, digestion media containing collagenase D and DNAse 1 was added to the samples. Samples were pipetted up and down for 3 cycles. EDTA was added (5 mM) and cells were filtered through 70 μm filters and 40 uM mesh and resuspended in Fc blocking buffer. Cells were washed and stained for FACS analysis.

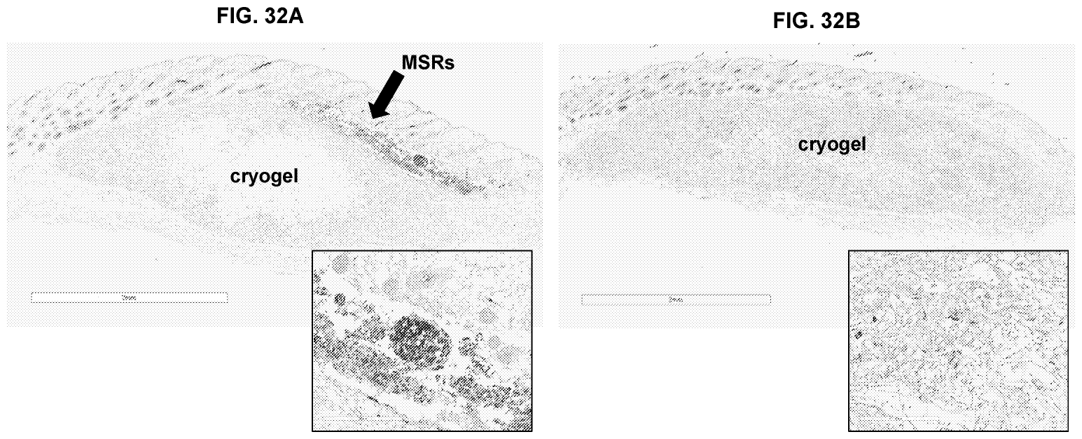

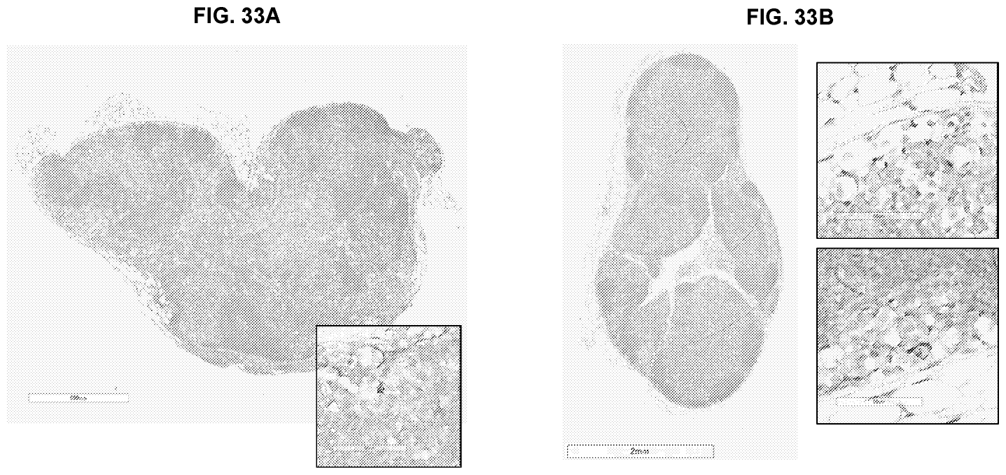

H&E and ISH Staining (FIGS. 32-33)

Skin/cryogel tissues, adjacent skin and draining lymph node were collected at necropsy, submersion fixed in 10% neutral buffered formalin and processed to paraffin. Sections were stained with hematoxylin and eosin (H&E) for histological evaluation. In situ hybridization to detect CAR transcript as well as Hs-PPIB (positive control and tissue quality control) and DAPB (negative control) genes was performed on formalin fixed paraffin embedded tissue sections. Positive PPIB and negative DAPB control probe sets were included to optimize preconditioning and ensure mRNA quality and specificity, respectively. The hybridization method followed known protocols using a 3,3′-Diaminobenzidine (DAB) chromogen. Briefly, 5 μm thick tissue sections were placed on glass slides, baked for 60 minutes and used for hybridization. The deparaffinization and rehydration protocol was performed using a stainer. Off-line manual pretreatment in 1×retrieval buffer was conducted. Optimization was performed by first evaluating PPIB and DAPB hybridization signal and subsequently using the same conditions for all slides. Following pre-treatment, the slides were transferred to a autostainer to complete the hybridization procedure including protease pretreatment; hybridization followed by amplification; and detection with HRP and hematoxylin counter stain. Slides were digitalized using a slide scanner and representative images captured.

Further Results

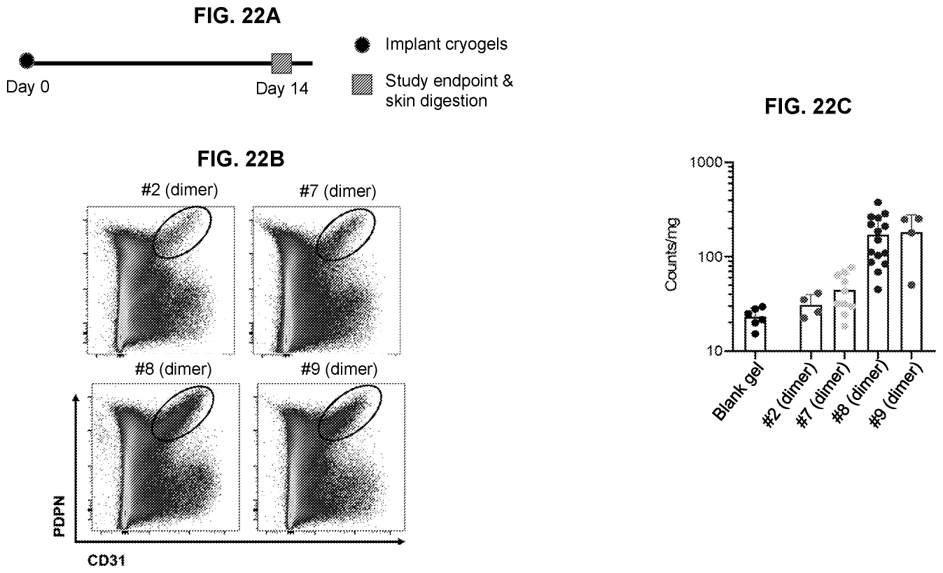

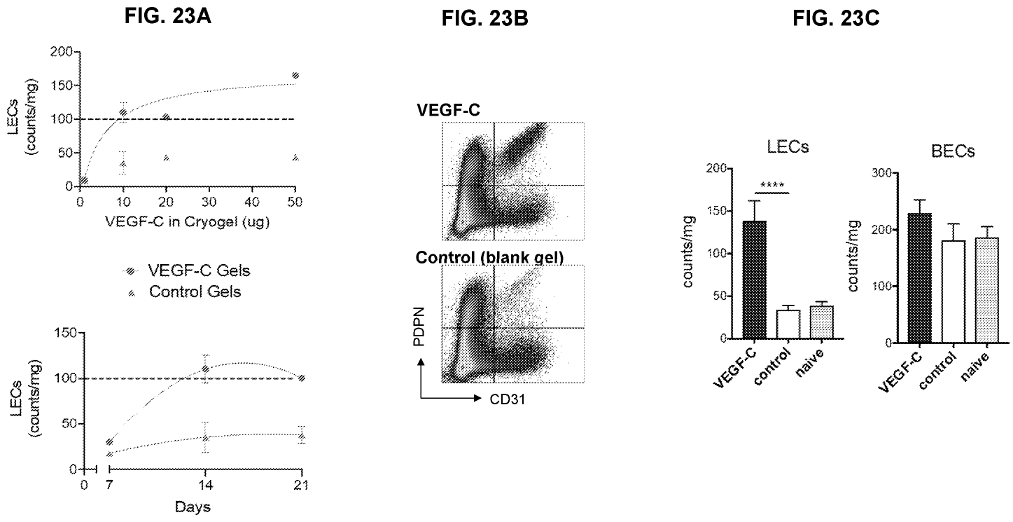

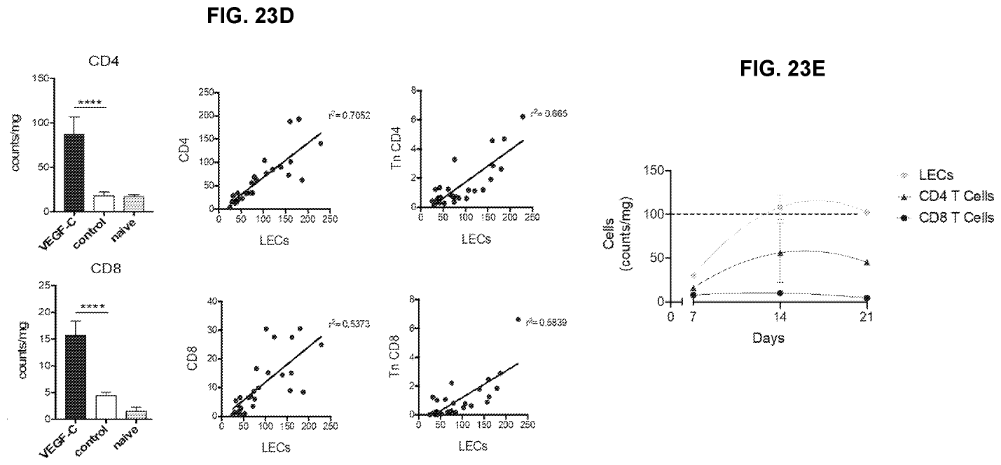

Cryogels (0.25 mg/ml laponite) containing 10 μg of different VEGF-C constructs were injected superficially into the dermis mice (N=5/group) at day 0. At day 14 mice were euthanized and the skin/cryogel (combined) were dissected for digestion and FACS analysis. Representative FACS plots of LECs (CD31⁺, PDPN⁺) are pre-gated on FSC-A/SSC-A, and CD45⁻ (FIG. 22). The results of FIG. 23 bear on the delivery of VEGF-C. Mice were injected with VEGF-C (N=12) or blank cryogels (N=10) synthesized as previously mentioned. Skin/gels from mice were harvested at timepoints day 7, 14, and 21, digested, and stained for FACS. LECs were gated on CD45⁻ CD31⁺ PDPN⁺. BECs were gated on CD45⁻ CD31⁺ PDPN⁻. CD4 and CD8 T Cells were gated on CD45⁺ CD11b⁻CD11c⁻Thy1⁺.

In FIG. 25A C57B16 Mice were injected with either VEGF-C cryogels (N=5) or blank cryogels (N=5) and evaluated for lymphangiogenesis at day 14. Additionally, NSG mice were injected with VEGF-C cryogels (N=5) to compare LEC proliferation between immunocompetent mice (C57B16) and immune-compromised mice (NSG). For FIG. 25B, NSG mice were injected on day 0 with either VEGF-C cryogels (N=5) or blank cryogels (N=5). On day 10, all mice were injected with PBMCs via their lateral tail vein. Seven days after PBMC injection skin/gel tissue was collected for digestion and FACS analysis. LECs were gated from human CD45⁻ mouse CD11b⁻ mouse CD31⁺ mouse PDPN⁺. CD4 and CD8 T cells were gated from human CD45⁺ mouse CD11b⁻ human CD3⁺. B Cells were gated human CD45⁺ mouse CD11b⁻ human CD3⁻ human CD19+.

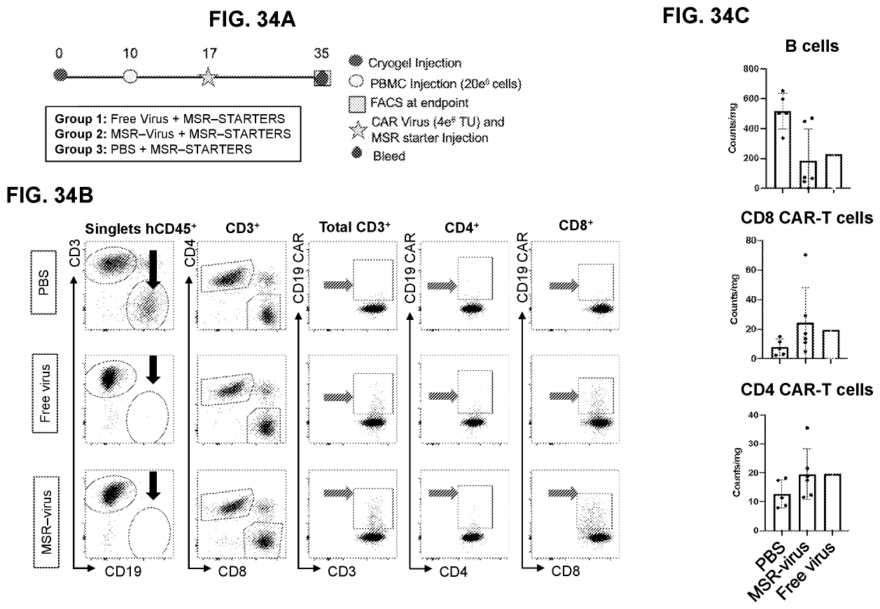

In FIG. 34, NSG mice (N=45) were injected with VEGF-C cryogels at day 0 and PBMCs at day 10 via tail vein injection. At day 17 mice were intradermally injected with either 1) PBS (N=15 total/5 for endpoint FACS analysis), 2) MSR with Construct 2 (10 μl, injection of 15 mg/ml MSR equivalent) followed by free CD19 CAR encoding lentivirus (10 μl injection containing 4.26e6 TU of virus at 1 hour post STARTERS injection) (N=15 total/6 for endpoint FACS analysis) or 3) MSR bound CD19 CAR encoding lentivirus mixed 1:1 with MSR with Construct 2 (20 ul single injection of 15 mg/ml MSRs) (N=15 total/5 for endpoint FACS analysis). On day 14, 3 or 4 mice group mice were euthanized and analyzed for lymphangiogenesis in the skin/gel area, while on day 20 and 35, mice were euthanized and skin/gel and spleen were collected for histological analysis to look for local and systemic transduced cells (N=3/group/timepoint). Mice were periodically bled for FACS analysis of circulating CD19 CAR+ cells (D25, 30, 35). Finally, mice were euthanized for FACS analysis of skin/gel and spleen on day 35 to look for CART expansion and B cell depletion.

### Example I: Functional Analysis of In Vivo CART Manufacturing

### Summary

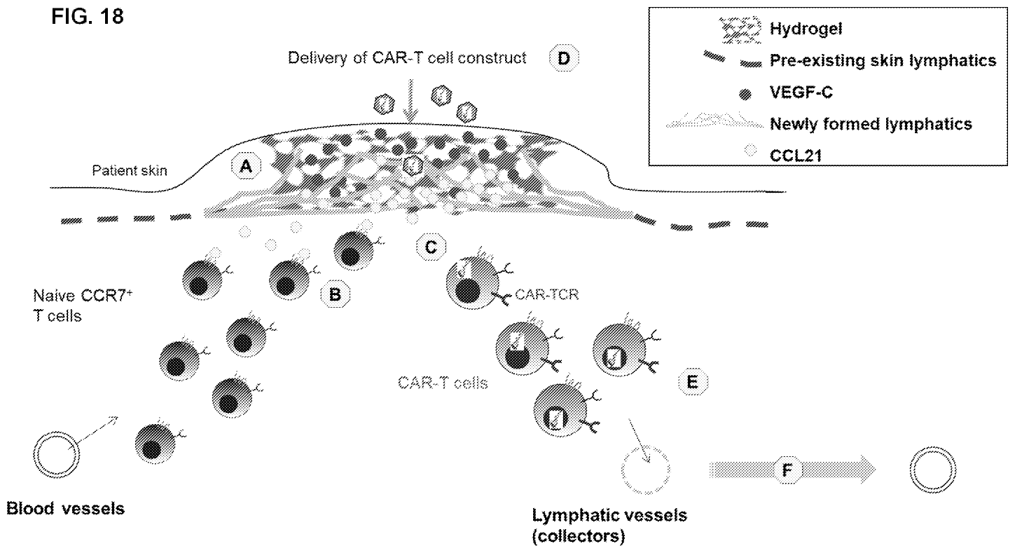

This Example describes an in vivo CART manufacturing process involving localized delivery of a cell recruitment factor to induce lymphangiogenesis and/or attract T cells, e.g., naïve T cells, that can be subsequently activated and transduced with a viral vector encoding a CAR to generate functional CART cells in vivo (FIG. 18). In some embodiments, a subject is administered (for example, locally) a cell recruitment factor via a cryogel, wherein the cell recruitment factor is VEGF-C(e.g., a slow-releasing VEGF-C protein cryogel formulation). Without wishing to be bound by theory, release of VEGF-C induces lymphangiogenesis of pre-existing skin lymphatic capillaries, activating lymphatic endothelial cells (LECs). The activated LECs secrete chemokines such as CCL21, which in turn recruit immune cells, e.g., naïve T cells, to the site of administration, e.g., to the site in the dermis on top of the gel. In some embodiments, after a period of 7 to 21 days, e.g., 14 days, the subject is administered (for example, locally) a viral vector encoding a CAR and a cell activation agent, delivered by mesoporous silica rods (MSRs), for transduction of T cells and generation of CART cells in vivo. Without wishing to be bound by theory, brief CD3 and CD28 activation, for example, using anti-CD3/anti-CD28 (for example, a bispecific antibody), promotes efficient transduction of T cells. Without wishing to be bound by theory, in some embodiments, these transduced T cells will return into systemic circulation via the skin lymphatics, lymph nodes and thoracic duct and will further expand in response to tumor antigen.

### Example 1: Generation and Characterization of VEGF-C Protein and Functional Variants

This Example describes the generation and characterization of various cell recruitment factors, including VEGF-C and functional variants thereof. Cell recruitment factors described in this Example may, for example, be used in cryogels for promoting lymphangiogenesis in a site in a subject prior to administration of a viral vector encoding a CAR.

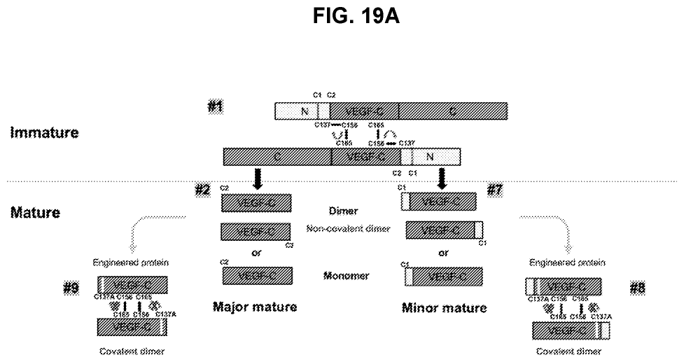

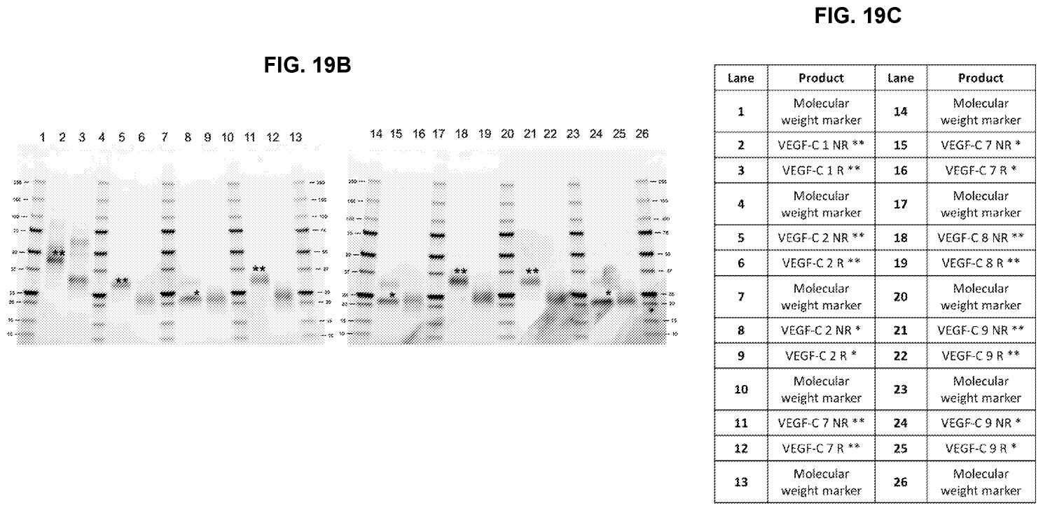

As shown in FIG. 19A, VEGF-C can exist in natural and modified forms. Immature VEGF-C(FIG. 19A, #1) is typically found intracellularly with N-terminal and C-terminal propeptide sequences. Once released, VEGF-C protein undergoes proteolytic cleavage and is present in the extracellular space as a major or minor mature form (FIG. 19A, #2 (SEQ ID NO: 731) or #7 (SEQ ID NO: 733), respectively) that is either a dimer or monomer. Stabilized dimers of the major mature (FIG. 19A, #9 (SEQ ID NO: 737)) and minor mature (FIG. 19A, #8 (SEQ ID NO: 735)) form of VEGF-C were engineered by inserting the C137A mutation into the sequences. As compared to the major mature forms of VEGF-C, the minor mature forms comprise an additional, short propeptide sequence (TEETIKFAA (SEQ ID NO: 740)) on the minor mature form N-terminus. Without wishing to be bound by theory, in some embodiments, this additional short propeptide boosts dimer formation as well as protein expression in both HEK293T and CHO MaKo cells.

HEK Production of VEGF-C Protein and Functional Variants

DNA was synthesized at GeneArt (Regensburg, Germany) and cloned into a mammalian expression vector using restriction enzyme-ligation based cloning techniques. The resulting plasmid was transfected into HEK293T cells. For transient expression of proteins, vectors for wild-type or engineered variants were transfected into suspension-adapted HEK293T cells using Polyethylenimine (PEI; Cat #24765 Polysciences, Inc.). The recombinant expression vectors were then introduced into the host cells and the construct produced by further culturing of the cells for a period of 7 days to allow for secretion into the culture medium. The produced constructs were then purified from cell-free supernatant, using immobilized metal ion affinity chromatography (IMAC). His-tagged proteins were captured by IMAC. The resin was washed before the protein was eluted. Finally, eluted fractions were polished by using size exclusion chromatography (allowing separation of aggregates, monomers and dimers). Purification analysis was done using SDS-PAGE (FIG. 19B-19C) under reducing and non-reducing conditions and aggregation content was determined using analytical size exclusion chromatography method.

As shown in FIG. 19B-19C, the immature VEGF-C with the full length propeptide (FIG. 19A, #1 (SEQ ID NO: 727)) was produced as a dimer (wells 2 and 3) and the removal of the propeptide led to the production of two major mature VEGF-C forms (#2 (SEQ ID NO: 731)), one non-covalent dimer form (wells 5, 6) and a monomeric form (wells 8, 9). A short N-terminal propeptide was left attached to the protein to generate a minor mature, wild type VEGF-C form (#7 (SEQ ID NO: 733)) in non-covalent dimer (wells 11,12) or in monomeric form (wells 15,16). Addition of the mutation C137A into the #2 major mature form of VEGF-C to generate the #9 major mature form with mutation of VEGF-C(SEQ ID NO: 737) led to the production of a covalent dimer (well 21, 22) and a monomer form (well 24, 25). Introduction of the C137A mutation to the #7 minor mature VEGF-C form to generate the #8 minor mature with mutation form of VEGF-C(SEQ ID NO: 735) led to the detectable production of a VEGF-C dimer only (wells 18,19). Without wishing to be bound by theory, in some embodiments, this #8 minor mature with mutation form of VEGF-C(SEQ ID NO: 736 or 735) may be suitable for large scale production.

Overall production yield using HEK cells was calculated and summarized in Table 21.

TABLE 21
Production yield of VEGF-C variants in HEK293T cell line

| | Physical | Final yield after IMAC |
|---|---|---|
| Variant | state | and SEC (ug/L) |
| 1 | Dimer | 117 |
| 2 | Dimer | 405 |
| | Monomer | 714 |
| 7 | Dimer | 1120 |
| | Monomer | 623 |
| 8 | Dimer | 1190 |
| 9 | Dimer | 869 |
| | Monomer | 125 |

CHO Production of the VEGF-C #8 Minor Mature Variant

A CHO MaKO manufacturing expression system was then used to produce the VEGF-C variant #8 (minor mature form with mutation, SEQ ID NO: 736 or 735). The gene encoding the target protein was introduced into an expression cassette driven by a CMV promoter in a plasmid expression vector. The vector was transfected in triplicate into CHO MaKO cells. For each transfection, 0.5 μg of plasmid were transfected into viable cells in medium. The transfected cells were seeded into cell culture medium with low concentration of folic acid in shake flasks. Cells were grown in a humidified shaker incubator. On day 3 post transfection, selection for stable transfectants was started. The cells went into a selection crisis and were recovered within 21 days. Then vials of the selected stable pools were frozen.

For production of the VEGF-C variant #8 (minor mature form with mutation, SEQ ID NO: 736 or 735), a fed-batch approach was used. A vial of the frozen cells was thawed. After recovery from thawing, cells were seeded into production cell culture medium in shake flasks. Cultures were grown in a humidified shaker incubator. Growth temperature was decreased on day 5 after seeding the culture. Feed solutions were added on day 3, 4, 5, 6, 7 and 10 after seeding. The culture was harvested on day 11 after seeding. Cells were separated from the cell culture medium by centrifugation and sterile filtering. The target protein was purified from the clarified cell culture supernatant and characterized as above. The production yield of the VEGF-C variant #8 (minor mature form with mutation, SEQ ID NO: 736 or 735) is summarized in Table 22.

TABLE 22
Production yield of VEGF-C variant 8 in CHO MaKO cell line

| | Physical | Final yield after IMAC |
|---|---|---|
| Variant | state | and SEC (mg/L) |
| 8 | Dimer | 17.5 |

Investigation of the In Vitro Activity of VEGF-C

The in vitro biological activity of VEGF-C and the variant forms produced above were investigated. The impact of VEGF-C on human dermal lymphatic endothelial cells (HDLECs), including sprouting and proliferation (FIG. 20A-20C), was measured. The various VEGF-C variants investigated in this example include: the immature VEGF-C with the full length propepitde (#1); the major mature form #2 as a monomer (2M) or a dimer (2D); minor mature form #7 as a monomer (7M) or a dimer (7D); the major mature form with mutation (C137A) #9 as a monomer (9M) or a dimer (9D); and the minor mature form with mutation (C137A) #8 as a monomer (8M) or a dimer (8D).

An overview of the experimental setup of the in vitro sprouting assay on HDLECs to test the biological activity of VEGF-C variants is shown in FIG. 20A. HDLECs were first seeded (p0) and passaged at 75% confluence until p4 or enough cells were obtained for experiments. About 1 mL of the HDLECs was then aliquoted into Eppendorf tubes, treated with 600 μg of VEGF-C protein, and vortexed. Each treatment was added to warmed media in a 6 well plate and incubated overnight at 37 C (5% CO₂). For imaging analysis, cells were washed and then fixed with 3.7% formalin-0.05% Triton X-100 solution (fixing buffer), then washed and coated with 0.1% Triton X-100 solution (permeabilization buffer). After washing twice with PBS, cells were coated with 0.05% Triton X-100 in 1% BSA solution (blocking buffer). DAPI and phalloidin staining was performed according to known protocols. After phalloidin staining, cells were washed twice with PBS, and imaged for tube formation.

In order to measure proliferation, a WST-8 assay was performed (FIG. 20B), in which WST-8 media was first prepared by diluting WST-8 solution 1:10 into MV media. Culture media was removed, and cells were incubated in WST-8 media. Following incubation, media was removed in triplicate from each well and added to a 96 well plate. Absorbance of each well at 450 nm was read using a spectrophotometer.

As shown in FIG. 20B, all VEGF-C mature forms (wild-type or mutant major and minor mature forms) led to similar levels of proliferation of HDLECs, which was improved relative to proliferation of HDLECs incubated with the immature VEGF-C with the full length propeptide (#1). As shown in FIG. 20C, the tube formation images show that the major and minor mature forms that are wild-type (#2 and #7, respectively) or comprise the C137A mutation (#9 and #8, respectively) stimulate better sprouting of HDLECs than the immature form (#1). Further, the dimeric forms (D) of the various VEGF-C forms appeared to demonstrate good in vitro activity (FIG. 20B-20C). Without wishing to be bound by theory, in some embodiments, the dimeric forms of the major and minor mature forms that are wild-type (#2D and #7D, respectively) or comprise the C137A mutation (#9D and #8D, respectively) comprise enhanced sprouting activity as compared to the monomeric forms.

### Example 2: Generation and Characterization of Cryogel containing VEGF-C

A cryogel containing VEGF-C or functional variants was generated and investigated. Laponite was added to the cryogel formation to result in a slow, more controlled release of VEGF-C.

To form the cryogel, alginate conjugates were formulated based on a previously described protocol (Koshy et al., Acta Biomater. 2018 January; 65:36-43) and mixed.

Norbornene ALinate (Alg-Nb)

One gram of Pronova UP MVG alginate was dissolved in 100 ml of 0.1 M 2-(N-morpholino)ethanesulfonic acid (MES) buffer overnight at room temperature under constant stirring. 280 μl of 5-norbornene-2-methylamine (norbornene) was then added to the alginate solution. 1464 mg of 1-ethyl-3-(3-dimethylaminopropyl)-carbodiimide hydrochloride (EDC) and 1085 mg of N-hydroxysuccinimide (NHS) were separately dissolved in 20 ml of MES buffer and added to the alginate-norbornene solution and allowed to react for 24 hours at room temperature. After 24 hours the solution was dialyzed in sequential 5 L salt baths (7, 6, 5, 4, 3, 2, 1, 0, 0, 0 g/L NaCl) for 3 hours at each concentration. Solution was then filtered twice (0.22m vacuum) and frozen at −80° C. overnight. Frozen solutions were then lyophilized for 5 days and stored at −20 C until use in experiments.

Tetrazine Alginate (Alg-Tz)

One gram of Pronova UP MVG alginate was dissolved in 0.1 M 2-(N-morpholino)ethanesulfonic acid (MES) buffer overnight at room temperature under constant stirring. 126 mg of (4-(1,2,4,5-tetrazin-3-yl)phenyl methanamine hydrochloride (tetrazine) was then added to the alginate solution. 1464 mg of 1-ethyl-3-(3-dimethylaminopropyl)-carbodiimide hydrochloride (EDC) and 1085 mg of N-hydroxysuccinimide (NHS) were separately dissolved in 20 ml of MES buffer and added to the alginate-tetrazine solution and allowed to react for 24 hours at room temperature. After 24 hours, solution was quenched with 311 mg of hydroxylamine for 30 min and centrifuged at maximum RPM for 10 minutes. Solution was then filtered (0.22 uM filter) and dialyzed in sequential 5 L salt baths (7, 6, 5, 4, 3, 2, 1, 0, 0, 0 g/L NaCl) for 3 hours at each concentration. Solution was then filtered twice (0.22 uM filter) and frozen at −80° C. overnight. Frozen solutions were then lyophilized for 5 days and stored at −20 C until use in experiments.

Cryogel Formulation

The Alg-Tz and Alg-Nb were then dissolved in DIH₂O at 20 mg/ml with a Thermomixer (37 C, 2000 rpm) for 1 hour. Separately, laponite was dissolved for 1 hour at 5 mg/ml concentration in DI-H₂O under constant mixing at room temperature. Solutions were then filtered (0.22 uM filter) under sterile conditions.

For cryogels not containing laponite, the Alg-Tz solution and Alg-Nb solutions were then added together in a 1:1 ratio along with the desired amount of VEGF-C and diluted to a final concentration of 10 mg/ml (10 μg VEGF-C/gel).

For cryogel formulations containing laponite, VEGF-C(3 mg/ml stock) and laponite (Laponite XLG) (5 mg/ml stock) solutions were incubated together at room temperature for 1 hour. Alg-Tz solution and Alg-Nb solutions were then added in a 1:1 ratio and diluted to a final concentration of 10 mg/ml (0.25 mg/ml final concentration of laponite, 10 μg VEGF-C/gel). 50p of the cryogel and VEGF-C mixture with or without laponite was immediately pipetted into PEEK molds and frozen at −20° C. overnight. Prior to injection, gels were thawed at room temperature in the molds and then placed into 16 ga syringe needles. Needles with gels were then placed on 1 ml syringes with 100 μl of sterile PBS.

In vitro VEGF-C release from alginate cryogels The release rate of VEGF-C from alginate cryogel formations comprising laponite was investigated in vitro. As described above, alginate liquid prepolymer is mixed with laponite and VEGF-C, then frozen and thawed again before injection to generate porous matrix (FIG. 21A).

First, the release of VEGF-C from alginate cryogels with and without laponite was measured. Different types of alginate gels containing VEGF-C(10 μg protein) were formed for use in the VEGF-C release assay: alginate nanoporous (gelification occurs without cryogelation so no macro-scale pores are formed), alginate cryogel (after freeze and cryogelation, porous), and alginate cryogel with 0.25% laponite. To perform the release assay, VEGF-C cryogels (10 μg protein+0.25 mg/ml laponite) were incubated at 37° C. in 1 ml of release buffer (1% BSA solution in PBS). Release buffer was completely removed and replaced at various timepoints throughout the experiment, ranging from 0 to more than 500 hours following the addition of the release buffer. Samples of release buffer that were collected were stored at −80° C. until thawed for use in a VEGF-C ELISA. As shown in FIG. 21B, the alginate cryogel with 0.25% laponite showed the most controlled and longest-lasting release of VEGF-C in vitro.

Next, the modulation of VEGF-C release in vitro from a 0.25% laponite alginate cryogel loaded with 10 μg or 50 μg VEGF-C, or a 0.5% laponite alginate cryogel loaded with 50 μg VEGF-C were measured. As demonstrated in FIG. 21C, about 30% of the total VEGF-C was released from the gels and the 0.25% laponite alginate cryogel showed the most controlled release profile.

A cryogel was injected subcutaneously in mice using a 16G needle. FIG. 21D demonstrates successful implantation of the cryogel in the skin of a mouse.

Investigation of VEGF-C Cryogels In Vivo

The ability of VEGF-C delivered via cryogel (0.25 mg/ml laponite) to implant and induce lymphangiogenesis and recruit T cells in vivo was investigated in mice. These series of experiments investigate the ability of different VEGF-C variants including those comprising a C137A mutation and VEGF-C variants produced by different methods to induce lymphangiogenesis. The ability of varying amounts of VEGF-C cryogel to induce lymphangiogenesis and recruit T cells in wild type mice and immunocompromised NSG mice injected with human peripheral blood mononuclear cells (PBMCs) was also investigated.

First, cryogels (0.25 mg/ml laponite) containing 10 μg of different VEGF-C forms, including the dimeric forms of the major and minor mature forms that are wild-type (#2 and #7, respectively) and the major and minor mature forms that comprise the C137A mutation (#9 and #8, respectively), were injected superficially into the dermis of mice (N=5/group) at day 0. At day 14 mice were euthanized and the skin/cryogel (combined) were dissected for digestion and FACS analysis (FIG. 22A). Specifically, tissues were harvested from mice, weighted, and cut into very fine pieces with scissors. Samples were then enzymatically digested under constant stirring of digestion media containing collagenase 4 and DNAse 1. Samples were then pipetted up and down. Following pipetting, digestion media containing collagenase D and DNAse 1 was added to the samples. Samples were pipetted up and down for 3 cycles. EDTA was added (5 mM) and cells were filtered through 70 μm filters and 40 μM mesh and resuspended in Fc blocking buffer. Cells were washed and stained for FACS analysis.

Representative FACS plots of lymphatic endothelial cells (LECs) (CD31*, PDPN+) isolated on day 14 post-injection of the cryogel were pre-gated on FSC-A/SSC-A, and CD45-(FIG. 22B) and show that in vivo lymphangiogenesis was induced. The amount of lymphangiogenesis in mouse skin was quantified as LEC counts per mg of tissue, as shown in FIG. 22C. The covalent dimers of the major and minor mature forms that comprise the C137A mutation (#9 and #8, respectively) resulted in higher amounts of LECs compared to the dimers of the major and minor mature forms that are wild-type (#2 and #7, respectively), and thus led to higher levels of in vivo lymphangiogenesis following cryogel implantation (FIG. 22C).

In addition, the functionality of the VEGF-C variant #8 (minor mature form with C137A mutation) produced in CHO MaKo cells as described above was investigated in vivo and compared to the same mutant form produced in HEK cells. Both #8 variants showed comparable activity in an in vitro HDLECs sprouting assay despite being produced in different cell types.

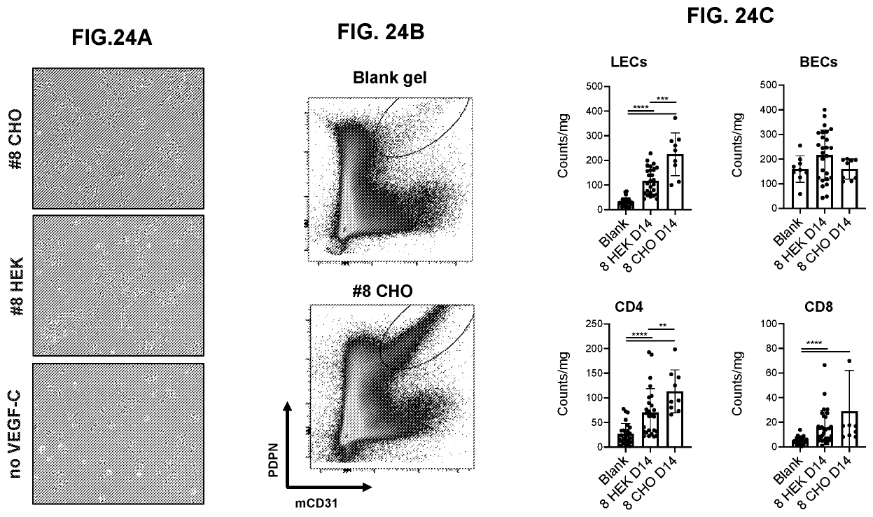

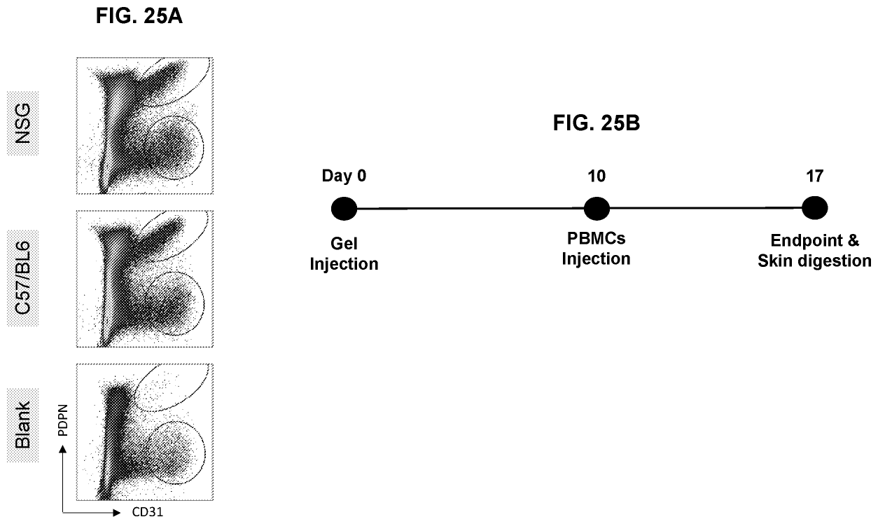

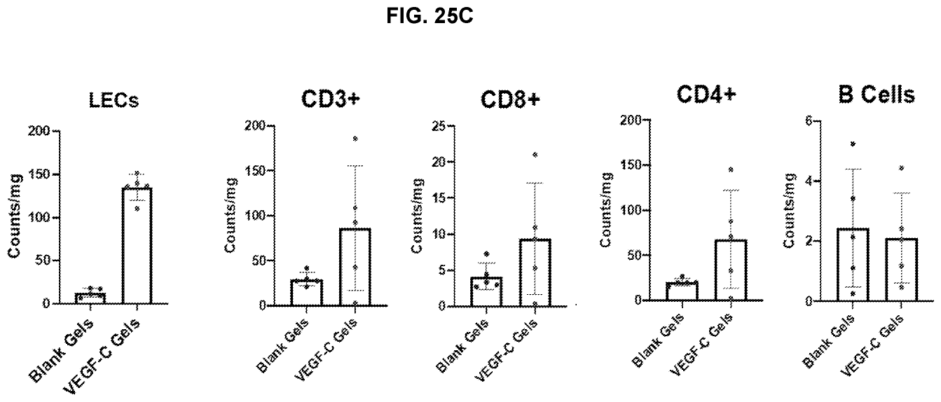

The VEGF-C variant #8 produced in CHO MaKo cells was able to induce lymphangiogenesis in vivo as shown in FIG. 24B, as compared to a blank cryogel control which does not comprise VEGF-C. Comparable bioactivity of the two #8 variants produced by the different cell types was then also confirmed in vivo by staining LECs after skin digestion 14 days after cryogel delivery (FIG. 24C). Lymphangiogenesis was quantified as total LECs counts/mg tissue in mice injected with a blank cryogel (no VEGF-C) or cryogel loaded with the #8 VEGF-C variant produced in HEK cells (#8HEK) or the #8 VEGF-C variant produced in MaKo cells (#8CHO). Blood vascular endothelial cells (BECs) (CD45−CD31+PDPN−) were not affected by the #8 variant VEGF-C delivery regardless of which cell type it was produced in. It was also observed that peak lymphangiogenesis corresponded to elevated LEC levels and peak immune infiltration of CD4 and CD8 T cells (CD45+) in the skin on top of the cryogels on day 14 after cryogel delivery with the #8 variant VEGF-C delivery regardless of which cell type it was produced in.

In addition, the response of skin lymphatics to 10 g VEGF-C delivered by alginate cryogels was measured. Mice were injected with VEGF-C alginate cryogels (N=12) or blank cryogels (does not comprise VEGF-C) (N=10) synthesized as previously mentioned. Skin/gels from mice were harvested as described above, at timepoints day 7, 14, and 21, digested, and stained for FACS. LECs were gated on CD45⁻ CD31⁺ PDPN⁺. BECs were gated on CD45⁻ CD31⁺ PDPN⁻. CD4 and CD8 T Cells were gated on CD45⁺ CD11b⁻ CD11c⁻ Thy1⁺. The number of cells per mg of tissue were also quantified.

FIG. 23A depicts the in vivo dose response of VEGF-C loaded into alginate cryogels (1, 10, 20, 50 μg) and lymphangiogenesis induction (represented as total lymphatic endothelial cells (LECs) counts/mg tissue, upper graph) and time course of lymphangiogenesis after delivery of 10 μg VEGF-C(lower graph). These results indicated that 10 μg of VEGF-C induced high in vivo lymphangiogenesis and that peak lymphangiogenesis was observed 14 days after subcutaneous cryogel delivery. Representative plots of LECs (CD45−CD31+PDPN+) and blood endothelial cells (BECs, CD45−CD31+PDPN−) staining isolated after skin digestion of C57/BL6 mice 14 days after gel implant (FIG. 23B) as well as quantification of endothelial cells as total cell counts/mg tissue (FIG. 23C) also indicated that 10 μg of VEGF-C induced high in vivo lymphangiogenesis compared to blank gel controls that did not comprise VEGF-C. Further, quantification represented as total cell counts/mg tissue of CD4+ T cells and CD8+ T cells demonstrated that T cell infiltration was also increased following 10 μg of VEGF-C alginate cryogel delivery, and LECs counts correlated with T cells infiltrates especially those of a naïve phenotype (CD62L+, CD44−). As shown in FIG. 23E, peak lymphangiogenesis was observed 14 days after subcutaneous VEGF-C alginate cryogel delivery, and peak T cell infiltrates were also observed on day 14.

The ability of VEGF-C to induce lymphangiogenesis in immunocompromised NSG mice and the ability of mouse LECs to efficiently recruit human peripheral blood mononuclear cells (PBMCs) was investigated. In FIG. 25A, C57B6 mice were injected with either VEGF-C cryogels/0.25% laponite (N=5) or blank cryogels (N=5) and evaluated for lymphangiogenesis at day 14. Additionally, NSG mice were injected with VEGF-C cryogels (N=5) to compare LEC proliferation between immunocompetent mice (C57B16) and immune-compromised mice (NSG). Representative flow cytometry plots of skin LECs and BECs staining in NSG, C57/BL6 mice 14 days after VEGF-C or blank cryogel delivery demonstrated increased LECs and thus higher in vivo lymphangiogenesis following delivery of VEGF-C cryogels in NSG and C57B6 mice (FIG. 25A). In FIG. 25B, NSG mice were injected on day 0 with either VEGF-C cryogels (N=5) or blank cryogels (N=5). On day 10, all mice were injected with PBMCs via their lateral tail vein. Seven days after PBMC injection, skin/gel tissue was collected for digestion and FACS analysis. LECs were gated on human CD45⁻ mouse CD11b⁻ mouse CD31⁺ mouse PDPN⁺. CD4 and CD8 T cells were gated on human CD45⁺ mouse CD11b⁻ human CD3⁺. B Cells were gated on human CD45⁺ mouse CD11b⁻human CD3⁻ human CD19⁺. These data demonstrate that CD3+ T cells, CD4+ T cells, CD8+ T cells, and B cells were all increased in the skin of the NSG mice administered the VEGF-C cryogels compared to the blank cryogel controls (no VEGF-C), and that T cells were the main cell type of PBMCs recruited in NSG mice.

Taken together, these data indicate that alginate cryogels comprising VEGF-C with or without laponite were capable of locally inducing lymphangiogenesis and T cell recruitment to the site of cryogel administration in vivo.

### Example 3: MSR Synthesis for Transduction of T Cells to Generate CART Cells

This Example describes the synthesis of mesoporous silica rods (MSRs). Mesoporous silica particles (MSPs) such as MSRs may be used, e.g., to assist with delivery of a viral vector encoding a CAR to a site in a patient that has undergone lymphangiogenesis due to administration of a VEGF-C cryogel described above.

MSR synthesis was performed by first dissolving poly(ethylene glycol)-block-poly(propylene glycol)-block-poly(ethylene glycol) avg Mn ˜5,800 (Pluronic P-123, 80.0 g, 487 mmol; Sigma) surfactant in 3 L of 1.6M HCl at room temperature, which was then heated to 40 C in a 5 L jacketed flask, and mechanically stirred via and overhead stirrer at a rate of 0-600 rpm. Tetraethyl orthosilicate (TEOS, 184 mL, 826 mmol; Sigma) was added in one portion over <5 min and was heated at 40 C with maintained stirring for at least 2 hours but most commonly 20 hours. The resulting slurry was heated to 80-130 C for 6-72 hours for hydrothermal treatment before being cooled to room temperature. The slurry was filtered in a Buchner funnel and was washed with deionized water followed by ethanol and air dried at room temperature. The resulting silica material was calcined in a furnace with a slow ramp temperature from room temperature to 550 C over 8 hours and then maintaining at 550 C for another 8 hours before cooling to room temperature to afford 47 g of mesoporous silica particles.

Changes in the stir rate may have changes in the microparticle aspect ratio. Varying the conditions of the hydrothermal temperature and duration are common pore size controllers for mesoporous materials.

Final mesoporous materials were characterized by light microscopy, Malvern Morphologi G3, scanning electron microscopy (SEM), thermal gravimetric analysis (TGA).

N,N,N-trimethylpropan-1-ammonium Functionalized Microparticles

Trimethoxysilylpropyltrimethylammonium chloride (3.61 mL, 6.51 mmol; 50% solution in methanol) was added to a slurry of 1.0 g mesoporous silica microparticles in 150 mL reagent ethanol and was heated to 75 degrees Celsius for 7 hours. After cooling to room temperature and the slurry was filtered and the particles were washed with deionized water followed by ethanol and then dried in an oven at 100 degrees Celsius for 20 hours to afford N,N,N-trimethylpropan-1-ammonium functionalized microparticles, referred to herein as “trimethylammonium MSRs”.

MSR Size Reduction

Stock MSR variants were roughly 100-200 μm in length after synthesis. MSRs were homogenized to reduce their size and improve injectability. In particular, to improve injectability through a 28.5 ga insulin syringe, dry MSRs were homogenized in a MP FastPrep-24 5G bead beating grinder and lysis system for 80 seconds (FIG. 30A). In FIG. 30A, size reduction was observed in terms of MSR length, which allows for injection through a smaller diameter needle into the intradermal space. MSRs were then autoclaved and stored at room temperature until use.

Loading of Lentivirus onto MSRs and Characterization of the MSR+Viral Vector

Homogenized MSR batches were resuspended at 10 mg/ml in ice-cold Tris-NaCl-EDTA buffer pH 7.5 (NTE buffer). The desired lentivirus stock (described below) was diluted in ice-cold NTE buffer to the desired total amount of transducing units (TU) for loading. The MSR suspension and the diluted virus were combined at ratios of 1:1 vol/vol and incubated on ice for 30 minutes. At least two wash steps in excess DPBS were performed to remove excess virus prior to functional in vitro tests, or in vivo use. For loading and retention studies (FIGS. 27-28), a 2.5 mg/ml MSR suspension was mixed 1:1 with a solution containing the indicated amount of virus in the figures and incubated for 30 minutes on ice. Virus amounts in loading solutions, bound to the MSRs, and released from the MSRs was quantified using a commercially available kit, e.g., a qRTPCR based kit, e.g., Lenti-X qRT-PCR Titration Kit. For release studies, the MSR-lentivirus complex was incubated in the media at 37° C., and the supernatant was collected at the indicated timepoints for qPCR analysis.

First, the loading capacity of MSRs with a GFP-encoding lentivirus was measured. Trimethylammonium MSRs were co-incubated with a GFP-expressing lentivirus at various amounts according to functional titers as determined by cell-based transduction assays. The amount of virus in three fractions was characterized, which include the viral loading solution (initial input added to the MSRs), MSR-bound virus (amount remaining bound to the MSRs after incubation and washing), and unbound virus (the amount in the solution remaining after co-incubation of MSRs and virus). MSR and virus were co-incubated for 30 minutes on ice, the supernatant was removed (Unbound Virus) and the MSRs were washed twice prior to assessing MSR-bound virus. The untouched viral loading solutions from each condition were also analyzed. As shown in FIG. 27A, the majority of the virus in the input viral loading solution was retained in the MSR-bound virus fraction after adsorption and washing as compared to the unbound virus. The amount of virus adsorbed to the MSRs also increased with the amount of virus in the viral loading solution. As depicted in FIG. 27B which graphs the percentage of viral loading solution versus the functional titer of virus, the calculated fraction of MSR-bound virus relative to the viral loading solution had a strong efficiency of loading and retention on the MSRs following adsorption and washing.

Next, retention of virus on MSRs was characterized. Specifically, lentivirus and MSRs were co-incubated for 30 minutes on ice, and the MSRs were washed twice. MSRs were then cultured in R10 medium containing 10% FCS or OpTmizer serum-free medium, and the input virus stock was incubated in media as well. The supernatant was removed at the indicated times after the start of incubation and analyzed for total virus content. As demonstrated in FIG. 28, the results indicated that the MSRs release only a fraction of the input virus over the first 18 hours (MSR-Virus in R10 media and MSR-Virus in OpTmizer media), compared to unbound virus controls (virus in R10 media and virus in OpTmizer media).

### Example 4: Transduction of T Cells with MSR and Viral Vector, and Optionally STARTERS, and Functional Testing

This Example describes the transduction of T cells in vitro using a viral vector encoding a CAR. Without wishing to be bound by theory, the viral vector was administered together with MSR (to promote controlled release of the vector), and optionally also STARTERS (to promote activation of the T cells).

In Vitro T Cell Transduction with MSR+Viral Vector+Optionally STARTERS

Human T cells were isolated from leukopaks using the Miltenyi Human Pan T cell isolation kit and frozen prior to use. Cells were thawed and plated in complete OpTmizer medium in the presence of STARTERS Construct 4 (Table 20, FIG. 48A). CD19 CAR encoding (also referred to as CAR19) virus, either as free virus or MSR-virus complexes were added into the culture, followed by one day of incubation. Cells were washed and plated for three additional days prior to use in characterization and functional tests.

CAR T Cell Characterization and Functional Testing

CAR T cells were analyzed for expression of the CAR receptor by staining with a CD19 CAR anti-idiotype antibody conjugated to PE and analysis by flow cytometry.

To measure CD19 CAR expression, the percentage of CAR+ cells was quantified at day 4 after transduction (FIG. 29A). Transduction efficiency of T cells in vitro was comparable for those T cells transduced with CAR-MSR or CAR-free (unbound virus) (FIG. 29A).

To measure function of CD19 CAR T cells transduced with MSR-bound virus or unbound virus, a Nalm6-Luc cell specific killing assay and an interferon-T (IFN-γ) release assay were used. Nalm6 (RRID: CVCL_0092) is a human acute lymphoblastic leukemia (ALL) cell line. Cells were grown in RPMI medium containing 10% fetal bovine serum and were grown in suspension. Cells were modified to express luciferase (Nalm6-Luc), so that their presence in co-cultures could be assessed by luciferase signal. Nalm6-Luc cells were co-cultured at various cellular ratios (Effector to Target rations (E:T Ratio)) with CD19-CARTs produced using free virus or MSR-virus complexes (FIG. 29B-29C). The luciferase signal at the end of a 1-day co-culture was used to calculate the percentage of input Nalm6 that had been killed by the CARTs. IFN-γ levels in the supernatant at the end of the co-culture were quantified using a commercially available kit.

Results from these assays indicate that specific killing activity (FIG. 29B) and IFN-γ release (FIG. 29C) during the co-incubation for 24 hours were comparable between CART cells transduced with the MSR-bound virus (CAR-MSR) and CART cells transduced with the free virus (CAR-free), showing that transduction by formulation with MSRs produced equivalently functional CARTs in vitro compared to conventional free virus transduction.

Next, the transduction efficiency of MSRs following homogenization was assessed. MSRs were homogenized as described above to allow for injection through a smaller diameter needle (FIG. 30A). As shown in FIG. 30B, standard trimethylammonium MSRs or homogenized MSRs were adsorbed with lentivirus and a dilution series of this complex was created and used to transduce T cells with a GFP-encoding lentivirus. Homogenization of MSRs did not substantially alter transduction performance in vitro.

Administration of Cryogel/Laponite Followed by MSR+ Viral Vector In Vivo

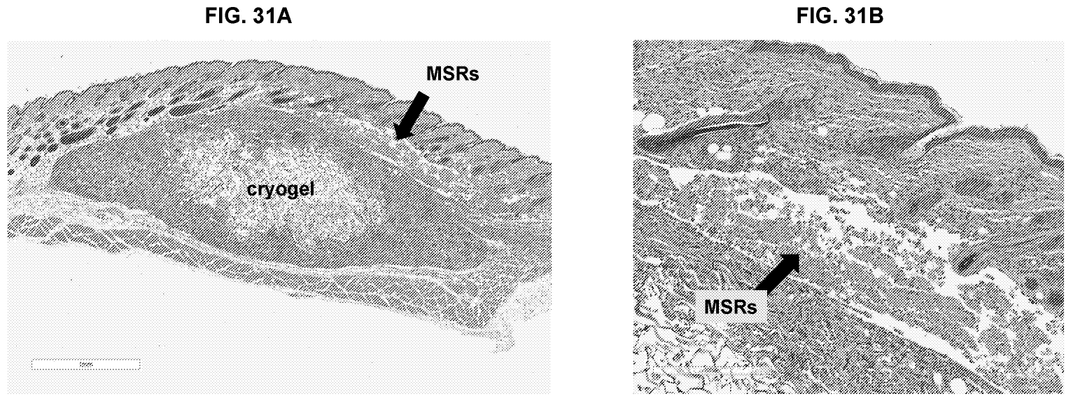

An experiment was designed to investigate localization and distribution of a cryogel injected into mice followed by injection of MSR-virus complexes. Blank alginate cryogels were injected subcutaneously and 7 days later viral particles (4e6 TU) free or bound to MSRs were injected in the intradermal space on top of the gel with insulin syringe (for MSR-virus group) or Hamilton syringe (for free virus). 72h after viral delivery, mice were euthanized, and tissues (skin and draining lymph node) were harvested for immunohistochemistry analysis. Skin/cryogel tissues, adjacent skin and draining lymph node were collected at necropsy, submersion fixed in 10% neutral buffered formalin and processed to paraffin. Sections were stained with hematoxylin and eosin (H&E) for histological evaluation. Slides were digitalized using a slide scanner and representative images captured. FIG. 31A-31B depict the hematoxylin and eosin (H&E) stained section of skin containing adjacent cryogel and MSRs.

In FIG. 31A, H&E stained sections demonstrated the location of the subcutaneous cryogel superficial to the panniculus muscle in the hypodermis. The MSRs appeared as lightly eosinophilic granular material admixed with mononuclear cells positioned at the dermal-hypodermal junction adjacent to the implanted cryogel (FIG. 31B for close up image).

FIGS. 32A-32B show in situ hybridization for CAR mRNA on sections of isolated mouse skin. In situ hybridization to detect CAR transcript as well as Hs-PPIB (positive control and tissue quality control) and DAPB (negative control) genes was performed on formalin fixed paraffin embedded tissue sections. Positive PPIB and negative DAPB control probe sets were included to optimize preconditioning and ensure mRNA quality and specificity, respectively. The hybridization method followed known protocols using a 3,3′-Diaminobenzidine (DAB) chromogen. Briefly, 5 μm thick tissue sections were placed on glass slides, baked for 60 minutes and used for hybridization. The deparaffinization and rehydration protocol was performed using a stainer. Off-line manual pretreatment in 1×retrieval buffer was conducted. Optimization was performed by first evaluating PPIB and DAPB hybridization signal and subsequently using the same conditions for all slides. Following pre-treatment, the slides were transferred to an autostainer to complete the hybridization procedure including protease pretreatment; hybridization followed by amplification; and detection with HRP and hematoxylin counter stain. Slides were digitalized using a slide scanner and representative images captured.

As shown in FIG. 32A, in situ hybridization to detect CAR mRNA transcript demonstrated robust signal within regions corresponding to the injected MSR in mice injected with MSR-bound virus. As depicted in FIG. 32B, in situ hybridization detected diffuse signal in cells infiltrating the cyrogel, as well as cells adjacent to it in the free virus condition. These data appear to support the notion that MSRs may maintain the virus localized in the dermis where the T cells are infiltrating the skin.

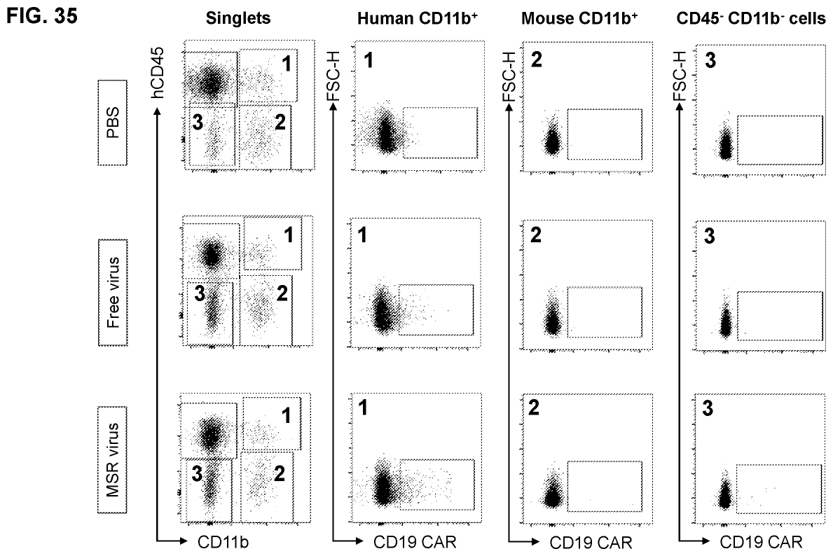

FIGS. 33A-33B show a mouse injected with MSR-virus had less CAR mRNA transcript positive cells in draining lymph node compared to the free virus group. In situ hybridization for CAR mRNA was performed as described above on sections of draining lymph node (dLN). This in situ hybridization detected only one CAR mRNA transcript positive cell within the dLN of mice injected with MSR-bound virus (FIG. 33A), while mice injected with free-virus showed a few CAR mRNA transcript positive cells in the subcapsular sinus consistent with local drainage of the virus or the cells from the site of cryogel implantation (FIG. 35B). This study demonstrated that MSP-virus formulations limited virus drainage to the draining lymph node, thereby decreasing potential off-site transduction and improving safety.

MSR Loading with Virus and STARTERS for In Vivo Use

STARTERS (specifically, STARTERS Construct 2, shown in Table 20, FIG. 37A) and trimethylammonium MSRs were co-incubated to allow adsorption of the cell activation agent onto the MSR surface. STARTERS Construct 2 protein was added to an 8 mg/ml trimethylammonium MSR suspension and incubated for 1 hour at 4° C. Loaded MSRs were washed three times and resuspended in DPBS to a final concentration of 15 mg/ml MSRs. NTE buffer solution containing CD19 CAR encoding lentivirus was mixed with a 10 mg/ml trimethylammonium MSR suspension and incubated for 30 minutes at 4° C. MSRs were washed twice and resuspended in DPBS to a final concentration of 15 mg/ml MSRs. Finally, MSRs were vigorously pipetted up and down, back loaded into insulin syringes, and immediately injected intradermally into mice.

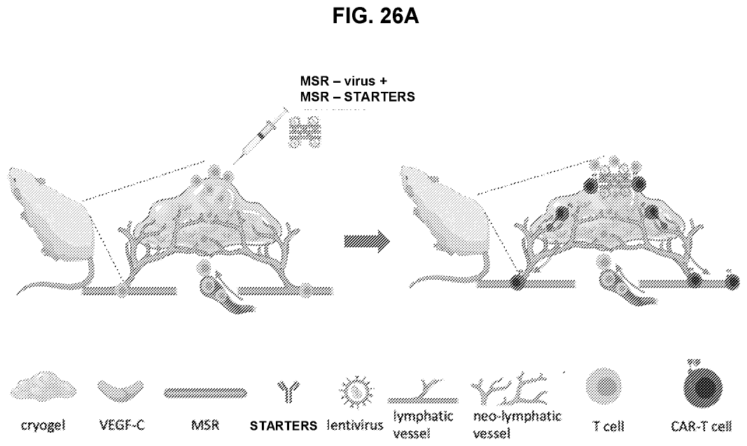

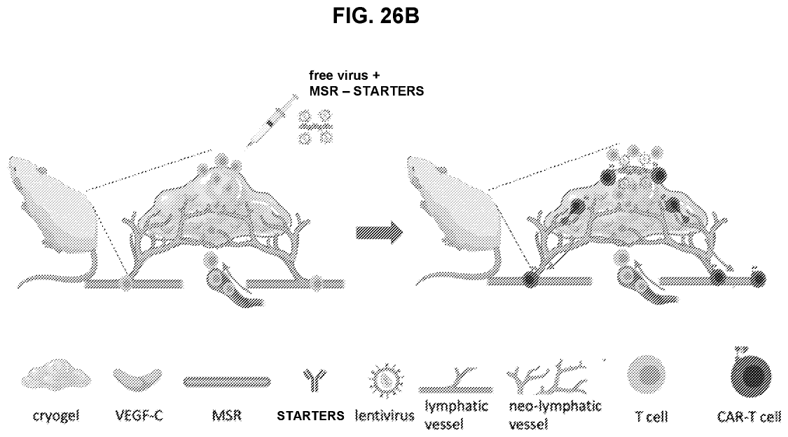

Taken together, these data indicate that MSR-bound virus, together with a STARTERS construct, was capable of efficiently transducing T cells to produce functional CART cells. Accordingly, contemplated herein is VEGF-C delivery in mouse skin generating a secondary priming site for T cells to be educated and transduced after injection of viral particles bound to mesoporous silica rods (MSRs) in combination with MSR-bound STARTERS (FIG. 26A), or free virus in combination with MSR-bound STARTERS (FIG. 26B).

### Example 5: In Vivo CART Manufacturing

A study was performed to demonstrate viral transduction of human T cells with a CD19 CAR encoding lentivirus in an in vivo mouse model. Specifically, a VEGF-C cryogel (0.25 mg/ml laponite) was administered to mice to promote lymphangiogenesis and recruitment of T cells. Next, a combination of a viral vector encoding CD19 CAR, MSR, and STARTERS was administered to activate and transduce the T cells in the area that underwent lymphangiogenesis.

### Methods

### Mouse Study and Flow Cytometry Analysis

NSG mice (N=45) were injected with VEGF-C cryogels at day 0 and PBMCs at day 10 via tail vein injection. Seven days later at day 17, mice were intradermally injected with either: 1) PBS (N=15 total/5 for endpoint FACS analysis), 2) MSR with STARTERS Construct 2 (10p injection of 15 mg/ml MSR equivalent, produced as described above) followed by free CD19 CAR encoding lentivirus (10 μl injection containing 4.26e6 TU of virus at 1 hour post STARTERS injection) (N=15 total/6 for endpoint FACS analysis) or 3) MSR bound CD19 CAR encoding lentivirus mixed 1:1 with MSR with STARTERS Construct 2 (20 μl single injection of 15 mg/ml MSRs) (N=15 total/5 for endpoint FACS analysis) (FIG. 34A). MSR-STARTERS was used for promoting T cell activation and T cell transduction. On day 14, 3 or 4 mice per group were euthanized and analyzed for lymphangiogenesis in the skin/cryogel injection area. On day 35, mice were euthanized and the spleen and blood were collected to determine if in vivo CD19 CAR encoding viral delivery elicited transduction of those T cells recruited by VEGF-C induced lymphangiogenesis and to look for local and systemic transduced cells (N=3/group/timepoint). Mice were periodically bled for FACS analysis of circulating CD19 CAR+ cells on days 25, 30, 35. Finally, mice were euthanized for FACS analysis of skin/cryogel and spleen on day 35 to look for CART expansion and B cell depletion. B cells depletion and CAR-T cells were also quantified in the spleen of the mice in all groups.

### ISH Staining of CAR

In situ hybridization using RNAscope 2.5 VS probe CAR 3UTR (catalog #438289) to detect CAR mRNA transcripts as well as 2.5 VS probe Hs-PPIB (positive control and tissue quality control (catalog #313909)) and 2.5 VS probe DAPB (negative control (catalog #3120390) was performed on blocks using reagents and equipment supplied by Advanced Cell Diagnostics (ACDBio) (Hayward, CA) and Ventana Medical Systems (Roche, Tucson AZ). Positive PPIB and negative DAPB control probe sets were included to ensure mRNA quality and specificity, respectively. The hybridization method followed protocols established by ACDBio and Ventana systems using a 3,3′-Diaminobenzidine (DAB) chromogen and optimized for study tissues. Briefly, 5 μm sections were baked at 60 degrees for 60 minutes and used for hybridization. The deparaffinization and rehydration protocol was performed using a Sakura Tissue-Tek DR5 stainer with the following steps: 3 times xylene for 3 minutes each; 2 times 100% alcohol for 3 minutes; air dried for 5 minutes. Off-line manual pretreatment in 1× retrieval buffer at 98 to 104 degrees C. was conducted for 15 minutes. Optimization was performed by first evaluating PPIB and DAPB hybridization signal and subsequently using the same conditions for all slides. Following pretreatment the slides were transferred to a Ventana Ultra autostainer to complete the ISH procedure including protease pretreatment; hybridization at 43 degrees C. for 2 hours followed by amplification; and detection with HRP and hematoxylin counter stain.

### IHC Staining for T Cells

Immunohistochemistry staining for CD3 including the deparaffinization and antigen retrieval steps were performed on a Ventana Discovery XT autostainer using standard Ventana Discovery XT reagents (Ventana, Indianapolis, IN) using a rabbit monoclonal antibody clone 2GV6 (Ventana catalog #790-431). Slides were deparaffinized then submitted to heat-induced antigen retrieval by covering them with Cell Conditioning 1 (CC1/pH8) solution according to the standard Ventana retrieval protocol. Slides were incubated with the primary antibody or a non-immune isotype-matched negative control. Visualization was obtained by incubation with Ventana Discovery OmniMap HRP reagent followed by Ventana Discovery ChromoMap 3,3′-Diaminobenzidine (DAB). Counterstaining was performed using Ventana Hematoxylin and Ventana Bluing reagent for 4 minutes each. Slides were dehydrated, cleared and coverslipped with a synthetic mounting medium.

### IF Staining Protocol Mouse Tissue for In Vivo CART- Multiplexing

On day 1, slides were washed and then blocked overnight at 4° C. On day 2, slides were incubated with AffiniPure Fab fragment goat anti-mouse IgG (H+L) and then with Alexa Fluor® 647-labeled anti-CD19 antibody overnight at 4° C. On day 3, slides were washed and then incubated with Alexa Fluor® 488-labeled anti-CD3 zeta antibody overnight at 4° C. On day 4, the slides were washed, counterstained with DAPI, and then washed again. Slides were mounted with cover glass, placed in the fridge for at least 24 hours before imaging.

### Results

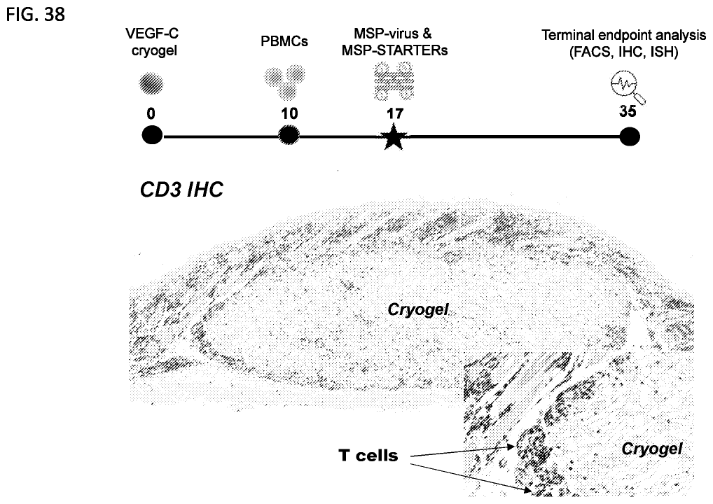

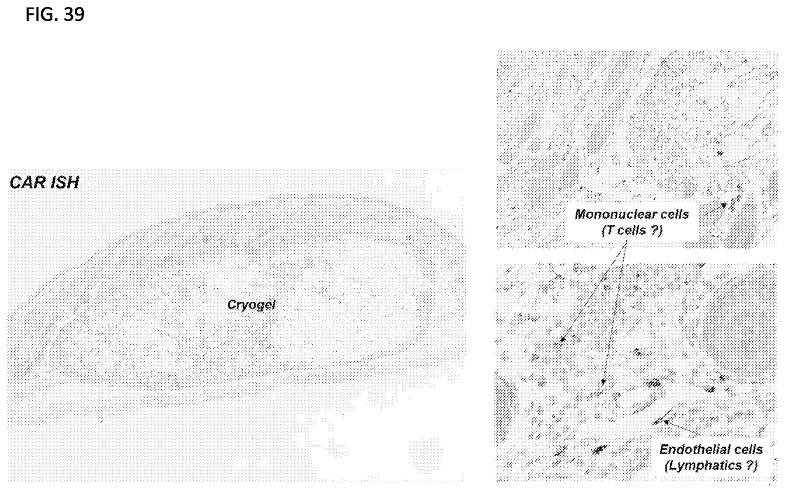

As demonstrated in FIG. 38, T cells were recruited efficiently around the implant site in the mouse skin and surrounding the gel. Results from the CAR ISH study showed that some mononuclear cells (from the phenotype likely T cells) in the area were transduced with the lentiviral vector that encodes CAR (FIG. 39). The expression of CAR RNA was also observed in some endothelial cells (likely lymphatics) (FIG. 39). However, CD19 CAR protein expression on the cell surface, analyzed by flow cytometry, was confirmed to be almost exclusively on T cells, with the exception of few human monocytic cells, indicating that there was minimal transduction of non-T cells by the free virus and MSR-virus (FIG. 35).

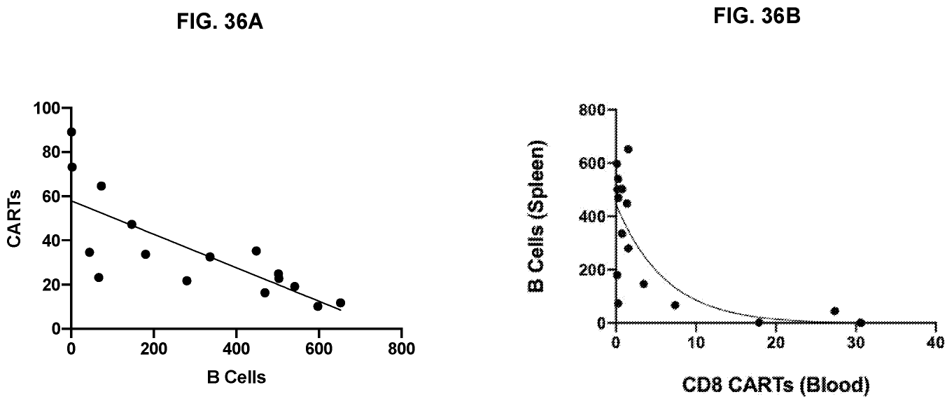

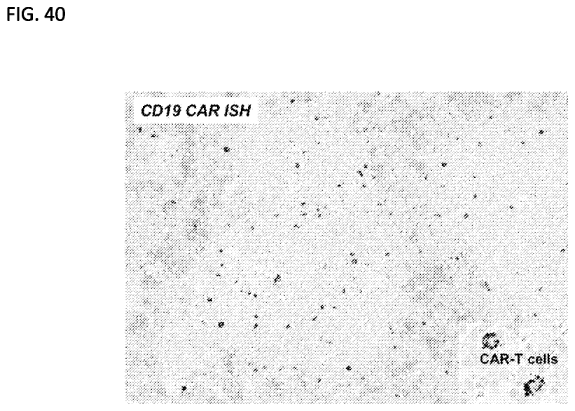

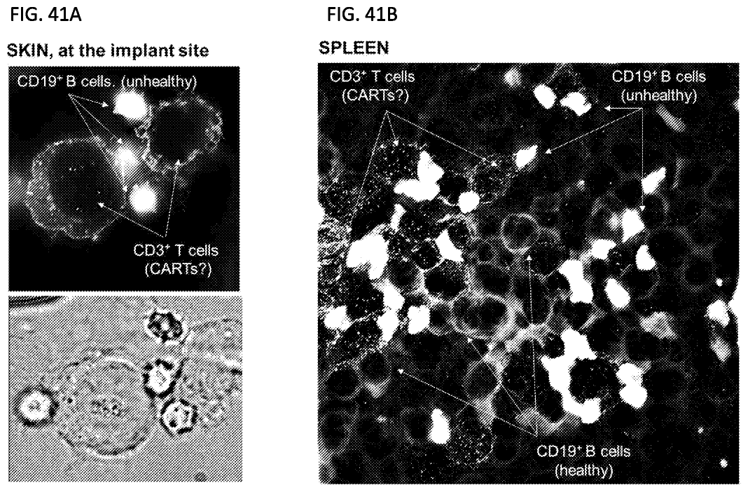

The locally generated CAR-T cells migrated to the spleen (FIG. 40). As shown in FIG. 34B, CAR-T cells as well as significant reduction of B cells were detected in those mice that received (a) free virus and MSR bound with STARTERS Construct 2 or (b) MSR bound CD19 CAR encoding lentivirus mixed 1:1 with MSR with STARTERS Construct 2. The number of B cells in the spleen inversely correlated with the number of CAR-T cells (which were mainly CD8+) in the spleen (FIGS. 34C and 36A) as well as in the blood (FIG. 36B), supporting that B cell depletion was caused by CD19-specific CAR-T cells generated in vivo. As shown in FIGS. 41A-41B, B cells in close proximity with T cells had a shrunken and unhealthy shape indicating cell death, while B cells that did not enter yet in contact with CD19 specific T cells showed a healthy phenotype (round and bigger cells with CD19 staining only at the cell surface). T cells stained with CD3 antibody (dotted staining) were likely CART cells, even though co-stain with CAR probe was not performed.

This study demonstrates that functional CART cells can be generated in vivo, following local administration of cryogel containing a growth/cell recruitment factor, e.g., VEGF-C, for induction of lymphangiogenesis and recruitment of T cells, which are then subsequently transduced by MSR-bound virus or free virus and MSR-bound STARTERS.

### Example 6: Additional Study Testing In Vivo CART Manufacturing

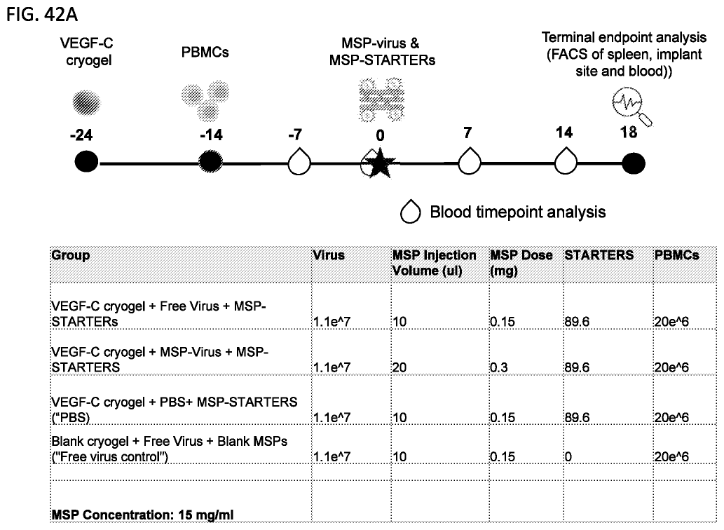

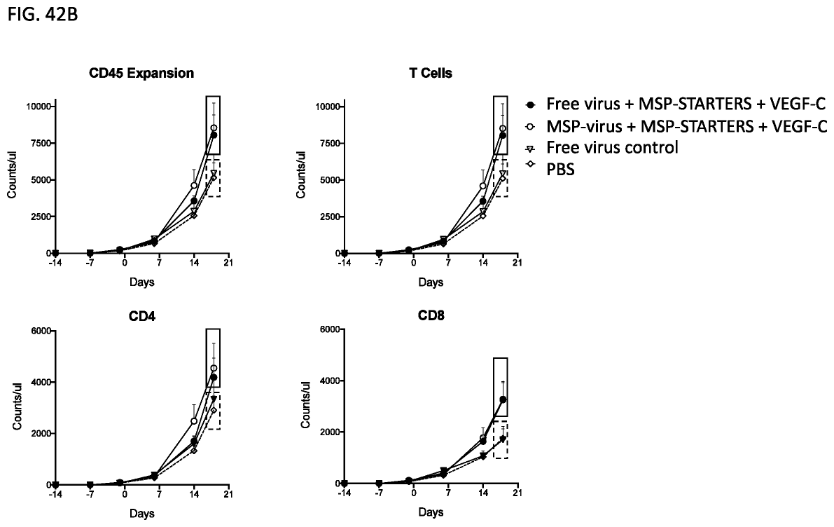

In this example, an additional study as laid out in FIG. 42A was conducted to test in vivo CART manufacturing. The methods are similar to the ones described in Example 5. Briefly, NSG mice were subcutaneously injected with VEGF-C cryogel (0.25 mg/ml laponite) at Day −24 and human PBMCs at Day −14. At Day 0, mice were intradermally injected with: 1) free virus and MSP with STARTERS Construct 2 (referred to as the free virus group), 2) MSP with virus and MSP with STARTERS Construct 2 (referred to as the MSP-virus group), or 3) PBS and MSP with STARTERS Construct 2 (referred to as the PBS group). A fourth group of mice received blank cryogel at Day −24 and free virus as well as blank MSPs at Day 0 (referred to as the free virus control group). The first two groups (the free virus group and the MSP-virus group) are referred to as full combination groups or groups receiving full combination treatments. The viral dose was increased from 4e6 TU (Example 5, FIG. 34A) to 1.1e7 TU (Example 6, FIG. 42A).

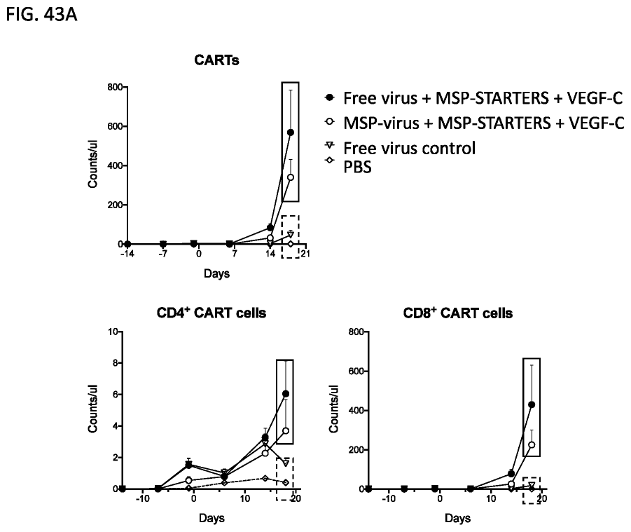

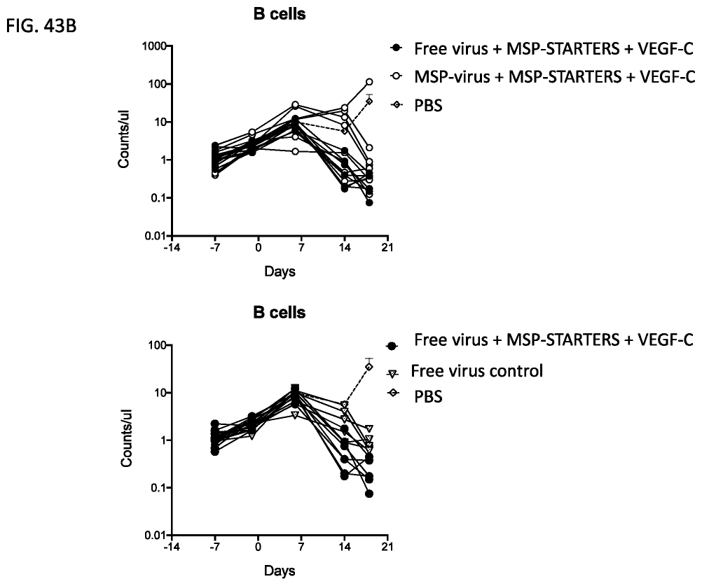

As shown in FIG. 42B, human CD45 circulating cells increased in number over time. Full combination treatments (solid line box, white and black circles) further boosted T cell expansion, particularly CD8+ T cells, compared to control mice (dotted line box, grey triangles, white rhombus). Different from the study described in Example 5 (virus injection 17 days after VEGF-C injection and 7 days after PBMC injection), in this new study, virus injection was postponed to 24 days after VEGF-C injection and 14 days after PBMC injection. This modification led to a higher consistency of CART cell generation, likely due to the higher density of T cells around the VEGF-C cryogel implant at this later time point (day 24). As shown in FIG. 43A, in vivo generated human CD3+ CAR+ T cells increased in number over time in mice treated with the full combination treatments (solid line box, white and black circles), particularly CAR-T cells of the CD8 phenotype, compared to control mice (dotted line box, grey triangles, white rhombus). Corresponding to an increase in CAR-T cell number, a decrease of B cells in the circulation was observed over time compared to PBS control (FIG. 43B, upper panel). The MSP-virus and free-virus groups performed similarly, while the free virus control group (FIG. 43B, lower panel) also showed partial B cells depletion, although not to the same extent as the full combination groups. Indeed, the number of circulating CAR-T cells in the mice from the free virus control group was not as high as the number in the full combination groups.

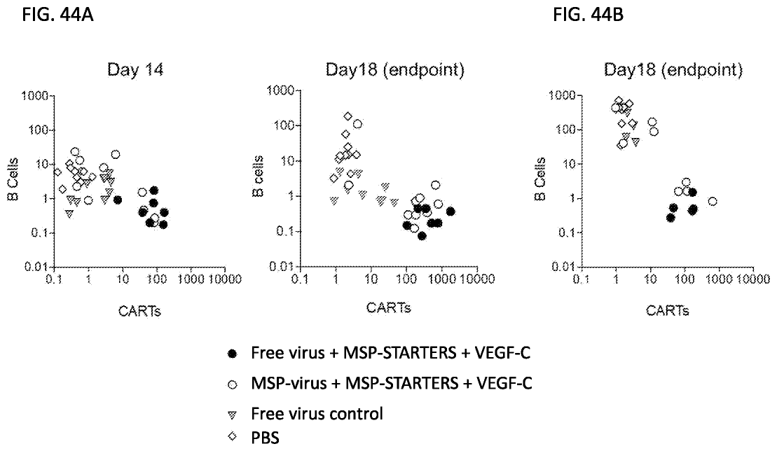

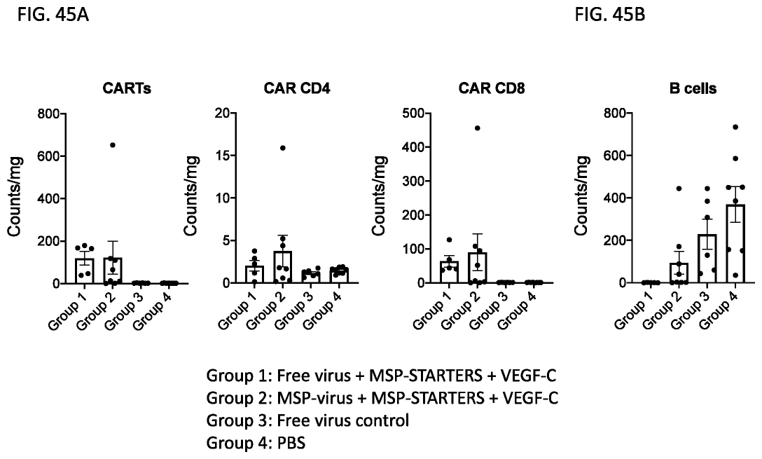

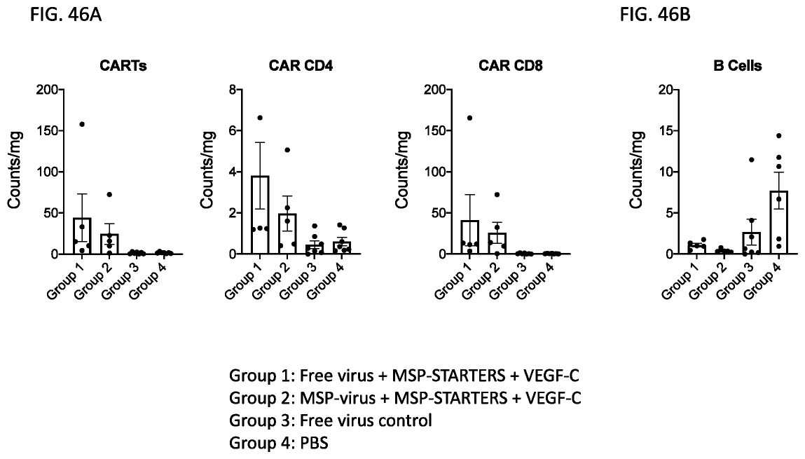

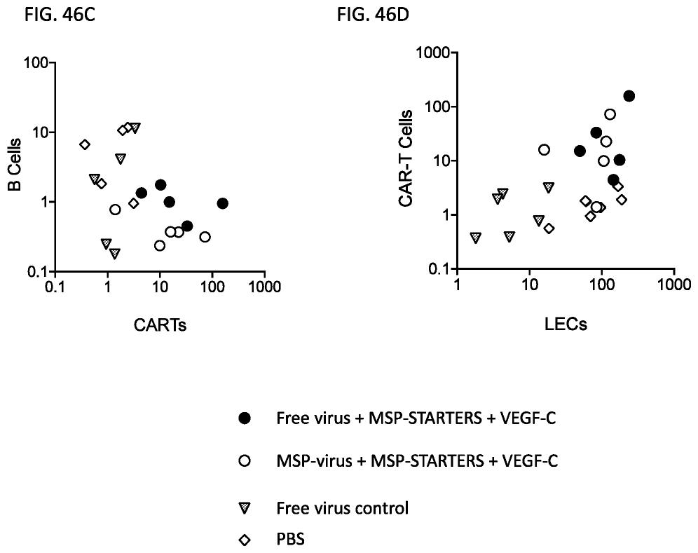

Consistent with results described in Example 5, this new study also showed a strong correlation between CAR-T cell expansion and B cell depletion in the blood (FIG. 44A) and in the spleen (FIG. 44B). While the free virus control group showed an intermediate level of B cell depletion in the circulation (FIG. 44A), this control treatment did not lead to effective B cell depletion in the spleen (FIG. 44B). The presence of CAR-T cells, especially from the CD8 phenotype, corresponded to B cells depletion in the spleen (FIGS. 45A-45B) and in the skin (FIGS. 46A-46C) of the treated mice. In the skin, CART cell expansion also correlated with lymphangiogenesis as measured by the number of lymphatic endothelial cells (LECs) (FIG. 46D).

In summary, this study demonstrates that the in vivo CART manufacturing methods described herein can be used to generate functional CARTs, the expansion of which correlates with B cell depletion.

### Example 7: Co-Delivery of Virus and STARTERS Using MSP

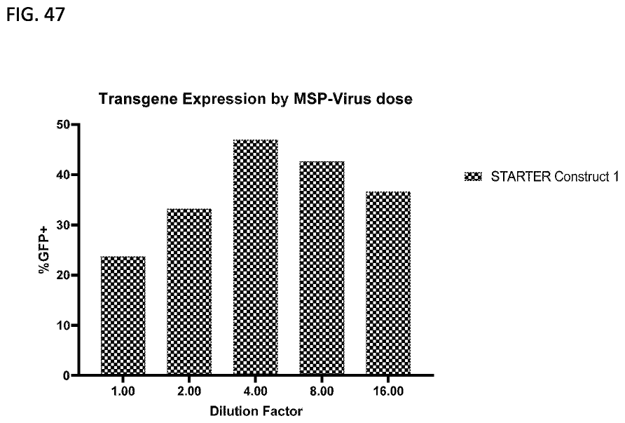

This example examines co-delivery of virus and STARTERS using the same MSP. Briefly, MSPs were simultaneously co-incubated with a lentiviral vector that encodes GFP and STARTERS Construct 1 (Table 20, FIG. 48A). After co-incubation and washing, the virus and STARTERS-loaded MSPs were serially diluted prior to incubation with T cells in a 96 well flat-bottom plate. As shown in FIG. 47, co-delivery of virus and STARTERS using MSP led to successful transgene expression in primary pan T cells.

### Example 8: Generation of STARTERS Molecules

This example describes the generation of multispecific molecules that comprise an anti-CD3 binding domain and a costimulatory molecule binding domain. In some embodiments, the costimulatory molecule binding domain binds to CD28, CD2, CD25, CD27, IL6Ra, IL6Rb, ICOS, or 41BB. Such molecules are referred to as STARTERS molecules.

A variety of configurations of anti-CD3× anti-CD28 or anti-CD3× anti-CD2 bispecific antibodies and multimer conjugates thereof were generated. Schema for these molecules are provided in FIGS. 48A-48B (Constructs 1-17, also referred to as F1 to F17; first generation STARTERS molecules). The sequences of Constructs 1-17 and their binding domains are disclosed in Table 19 and Table 20.

In addition, second generation STARTERS molecules were generated to test binders targeting different co-stimulatory molecules (e.g., CD25, IL6Rb, CD27, 41BB, ICOS, or CD2). Different anti-CD3 binders (binders based on ANTI-CD3 (1) or ANTI-CD3 (2)) were also compared. All the second generation STARTERS molecules (FIG. 49A) have the configuration shown in FIG. 49B. The sequences of the different binders of second generation STARTERS molecules can be found in Table 19.

Without wishing to be bound by theory, reducing the binding of STARTERS molecules to FcR may decrease or prevent unwanted killing of FcR-expressing cells by T cells. Third generation STARTERS molecules were generated by introducing D265A/N297A/P329A substitutions (EU numbering according to Kabat) (“DANAPA”) in the IgG1 Fc region. In addition, different anti-CD3 binders (binders based on ANTI-CD3 (1), ANTI-CD3 (2), or ANTI-CD3 (3)) and different anti-CD28 binders (binders based on ANTI-CD28 (1) or ANTI-CD28 (2)) were also compared (FIG. 50A). All the third generation STARTERS molecules have the configuration shown in FIG. 50B. The sequences of third generation STARTERS molecules and their binding domains can be found in Table 19 and Table 20.

The STARTERS molecules were shown to mediate transduction of T cells with a lentiviral vector that encodes a CAR.

Exemplary methods for assaying STARTERS activity are disclosed, e.g., in Examples 16-19 and 22-23 of PCT/US2021/019889, incorporated by reference herein in its entirety.

### Example J: Use of a STARTERS Molecule in In Vivo CART Manufacturing

This example describes the characterization of a STARTERS molecule for use in in vivo CART manufacturing. This STARTERS molecule is an anti-CD3/anti-CD28 bispecific molecule comprising an anti-CD28 antibody fused to an anti-CD3 scFv (FIG. 51A). This STARTERS molecule comprises a heavy chain comprising the amino acid sequence of SEQ ID NO: 726 and a light chain comprising the amino acid sequence of SEQ ID NO: 728. The Fc region of this STARTERS molecule comprises L234A/L235A/S267K/P329A mutations, numbered according to the Eu numbering system.

In a first in vitro study, the STARTERS molecule was tested for its ability to mediate T cell activation and transduction, when delivered via MSPs. Briefly, isolated T cells were thawed and resuspended at 1e6 cells/mL in serum-free T cell media with 100 units/mL of IL2 before being added to a 96-well plate. 20 mg of each MSP batch was resuspended at 30 mg/mL in Dulbecco's PBS (Thermo: #14190144) and 1.5 mg of MSP (or equal volume of DPBS) was added to Eppendorf tubes. To the same Eppendorf tubes, 2.1 μg of STARTERS was added. Lentivirus was thawed on ice and diluted to 1.4e8 TU/mL with DPBS before 7e7 TU of virus was added to each of the aforementioned tubes. After the mixtures were incubated at 4° C. for one hour, they were serially diluted with T cell media before being administered to plated T cells. Components were co-cultured with T cells for three days before T cell activation and transduction were evaluated using flow cytometry.

Two independently produced batches of MSPs were loaded with lentivirus and the STARTERS molecule prior to co-culturing with T cells in vitro. Delivering the STARTERS molecule bound to MSPs enhanced activation and transduction as compared to soluble delivery of the molecule, particularly in more dilute conditions (FIGS. 51B and 51C). The MSP-virus-STARTERS complexes were co-cultured with T cells for three days before a flow cytometry analysis was performed to examine CD25 expression as an activation readout (FIG. 51B) and GFP expression to measure transduction (FIG. 51C). The data show that complexes formed with either batch of MSPs were capable of activating T cells and mediating T cell transduction. It is worth noting that activation and transduction with MSPs shows a bell-shaped response. Without wishing to be bound by theory, high concentrations of MSPs in T cell cultures can have negative impacts on the T cell viability, and subsequently, can hamper activation and transduction efficiency.

A second in vitro study tested whether size reduction of MSPs, through either sonication or bead homogenization, would impact MSP performance. Isolated T cells were thawed and resuspended at 1e6 cells/mL in serum-free T cell media with 100 units/mL of IL-2 before being added to a 96-well plate. 60 mg of MSPs were resuspended at 30 mg/mL in DPBS. A portion of MSP solution was size-reduced by bead homogenization. Additionally, 30 mg of MSPs were resuspended at 5 mg/mL in sterile water, and then sonicated using a Q125 system (qSonica) sonication probe at an amplitude of 40% for a duration of two minutes (sonication performed in 15 sec intervals with 30 sec breaks). After sonication, MSPs were centrifuged to aspirate water and resuspended at 30 mg/mL in DPBS. 1.5 mg of full-sized or size-reduced MSPs were added to fresh Eppendorf tubes and an equal volume of DPBS was added to a separate Eppendorf tube as a soluble virus control. Then, 1.6 μg of the STARTERS molecule was added to MSPs or DPBS before 8e7 TU of thawed virus was added. The mixtures were incubated at 4° C. for one hour before serially diluted with T cell media and added to plated T cells. Components were co-cultured with T cells for three days before T cell activation and transduction were evaluated using flow cytometry.

GFP-encoding lentivirus and the STARTERS molecule were loaded onto full-size or size-reduced MSPs, where size reduction was achieved using bead homogenization or sonication of MSPs. The MSP-virus-STARTERS complexes were co-cultured with T cells for three days before flow cytometry analysis was performed for CD25 expression (activation) and GFP expression (transduction). Size reduction of MSPs did not negatively impact in vitro potency with respect to T cell activation (FIG. 52A) and transduction (FIG. 52B), and sonicated MSPs demonstrated the greatest peak transduction efficiency of MSP conditions tested.

Next, an in vivo study (FIG. 53A) was conducted to examine the use of the STARTERS molecule in in vivo CART manufacturing. Briefly, VEGF-C loaded cryogels (“injectable 1” in FIG. 53A) were injected at day −16 followed by a 20e6 human PBMC injection at day −14. 14 days later, different combinations of CD19 CAR-encoding virus and STARTERS molecules co-loaded onto MSPs (“injectable 2” in FIG. 53B) were injected intradermally above the cryogel location. Flow cytometry analysis was performed on blood from weekly bleeds and expansion of lymphocyte populations was assessed. On day 18, mice with significant CAR-T expansion were euthanized and circulating lymphocytes were harvested via cardiac puncture, analyzed for CAR % and cell count, pooled within the groups, and adoptively transferred into mice which had been challenged with 1e6 NALM6 cells 4 days prior. Weekly bleeds and bi-weekly luminescence imaging of tumors were performed until end of study. The aims of this study were to 1) elucidate the necessity of single components within each injectable, and 2) demonstrate the ability of in vivo manufactured CAR-Ts to control tumor burden.

As shown in FIG. 53C, in vivo manufactured CAR-Ts, when adoptively transferred into NALM6 tumor bearing mice, were able to recognize their target CD19 receptor and deplete B cells in these mice. As shown in FIG. 53D, tumors treated with transferred PBMCs from donor mice with no CARTs (“PBMC control” in FIG. 53D) showed comparable growth kinetics compared to untreated NALM6 tumors (“NALM6” in FIG. 53D). Mice treated with a dose of 3e5 CAR-Ts manufactured using free virus (“Free virus” in FIG. 53D) or MSP-delivered virus (“MSP virus” in FIG. 53D) show reduced tumor burden. These data show that in vivo-generated CARTs demonstrated strong anti-tumor activity in vivo.

Furthermore, expansion of in vivo-generated CARTs in circulation was examined. 13 days after intradermal injection of injectable 2 at the cryogel site, mice treated with a combination of VEGF-C cryogel, virus (MSP-deliver or free), and MSP-STARTERS showed comparable CART, shown by similar CAR percentage in total circulating T cells (FIG. 53E) and counts/μl of CART cells in blood (FIG. 53F). Mice treated without full treatment combination showed lower levels of CAR-T number and CAR+% (FIGS. 53E and 53F). 18 days after intradermal injection of injectable 2, despite similar amount of circulating T cells (FIG. 53H), mice treated with virus and STARTERS co-loaded onto MSPs had a higher CAR-T number (FIG. 531) and CAR+% (FIG. 53J) compared to mice treated with MSP-delivered STARTERS in combination with free virus. The day-18 data were used to determine the CAR+ cell number after pooling of blood lymphocytes to determine the number of cells dosed into mice bearing Nalm6 tumors.

FIGS. 53K and 53L show results from combined flow cytometry analysis of blood from mice used for adoptive transfer and remaining mice enrolled in the study. No significant difference in the number of circulating T cells was observed between groups (FIG. 53K). Full combination of injectable 1 and injectable 2 induced the highest absolute counts of CARTs in circulation (FIG. 53L).

### Example K: Use of HA-Hydrogel for Sustained Release of VEGF-C

This example describes the use of hyaluronic acid (HA)-hydrogel for sustained release of VEGF-C protein to achieve lymphangiogenesis and T-cell localization at an injection site.

### Example 1: Functionalization of Hyaluronic Acid

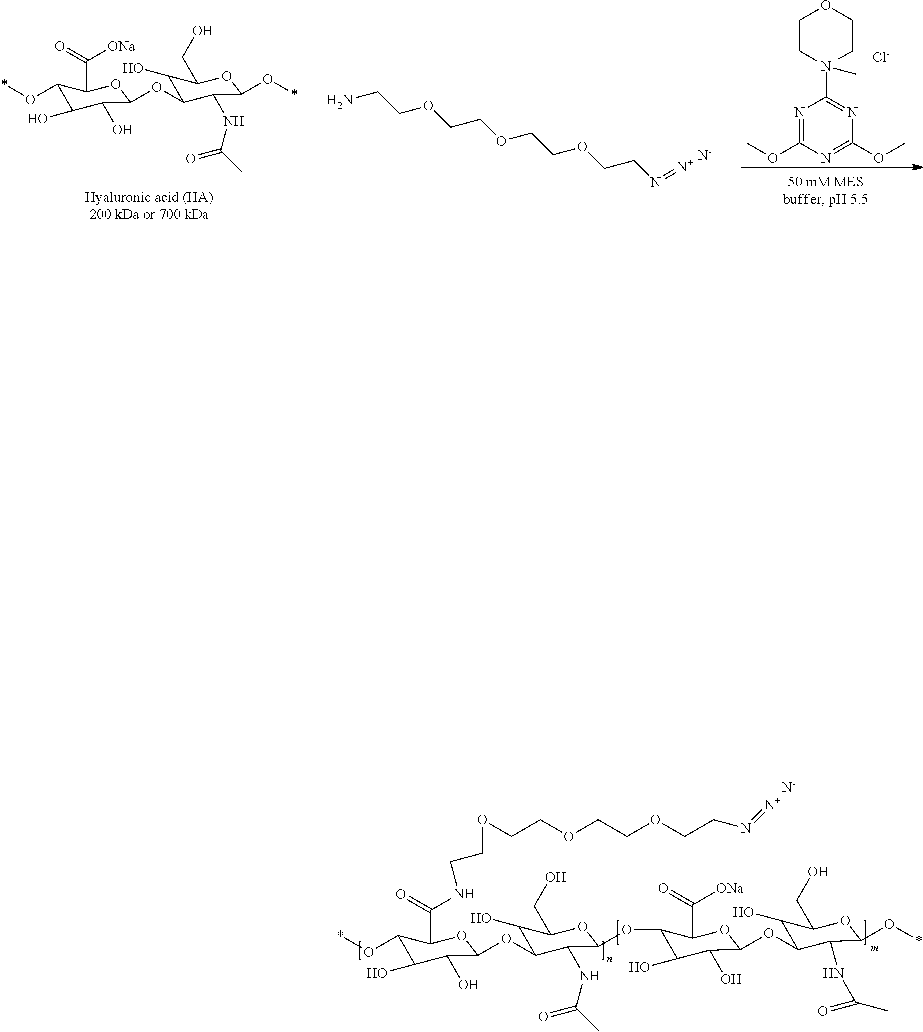

This example describes the synthesis of an azide functionalized hyaluronic acid, which is reacted with a crosslinking moiety to form a hydrogel.

### Synthesis of Hyaluronic Acid Intermediate [HA-N3]

Hyaluronic acid sodium salt is a linear polymer consisting of repeating dimeric units of glucuronic acid and N-acetylgalactosamine, with a repeating unit molecular weight of 401.3 Da. In this example the moles of hyaluronic acid reported refers to the moles of repeating unit and the equivalents of reagents used in the reaction with hyaluronic acid are reported relative to the moles of hyaluronic acid repeating unit. The average molecular weight of the polymer determines the average number of repeat units per polymer strand. Hyaluronic acid sodium salt was obtained from supplier Lifecore Biomedical with label HA700K-5 having a nominal average molecular weight of 700 kDa and may vary from batch to batch.

### Synthesis of 700 KD [HA-N3]-16%

A solution of hyaluronic acid, sodium salt (nominal average molecular weight 700 kDa; 258.9 mg, 0.62 mmol; Lifecore Biomedical, LLC; product number HA700K-5) was fully dissolved in 37.5 mL of MES buffer (50 mM, pH 5.5). To this solution was added 4-(4′,6′-dimethoxy-1′,3′,5′-triazin-2′-yl)-4-methylmorpholin-4-ium chloride (DMTMM, 298.1 mg, 1.077 mmol, 1.736 eq), followed after 5 min by addition of 11-azido-3,6,9-trioxaundecan-1-amine (201 mg, 0.921 mmol). The reaction was stirred overnight. A crude reaction mixture was filled into a dialysis membrane (MWCO 50 kDa), and dialyzed 1-3 days against 0.25 M NaCl, with several changes of the dialysis solution, followed by 1-3 day's dialysis against deionized water, also with multiple changes of the dialysis solution. Upon completion, the sample was removed from the dialysis tubing, flash-frozen, and lyophilized to get 700 kDa [HA-N3]-16%.

¹H NMR (400 MHz, D₂O) δ 4.45 (bs, 1.71H), 4.0-3.1 (m, 16H), 1.95 (s, 3H).

DOSY-NMR. One dimensional diffusion ordered NMR spectra (DOSY) were collected using the stimulated echo with one spoil gradient pulse sequence (stegp1s1d) on a Bruker AVANCE III 400 MHz (for ¹H-NMR) instrument with 5 mm DCH cryoprobe. The diffusion time and the diffusion gradient time were set to 50 ms and 4 ms, respectively. Two spectra were collected with gradient strength (gpz6) set to 2% and 95%. Comparison of the two spectra showed no differences apart from the solvent peak, indicating no small molecule impurities were present in the polymer.

Elemental analysis of a purified sample showed the following elemental content—C: 38.9%: H: 5.42%; N: 4.85%.

The degree of substitution of the [HA-N₃] is defined as the % of repeat units in which the carboxylate moiety has undergone reaction to give the depicted amide. Elemental analysis was used to determine the degree of substitution. The [% C/% N] ratio determined by elemental analysis of a purified sample is entered into the following formula to provide the degree of substitution. Where y=[(% C)/(% N)]) then:

$$Degree of substitution = 100 \times \frac{\frac{14 \times 12.01}{14.01 \times y} - 1}{4 - \frac{8 \times 12.01}{14.01 \times y}}$$

In this example, the degree of substitution (DS) of [HA-N3] was 16%.

This 700 kDa hyaluronic acid, functionalized with 16% of the azide linker is labeled 700 kDa [HA-N3]-16%.

### Synthesis of 200 KD [HA-N₃]-24%

In another aspect, hyaluronic acid, sodium salt (nominal average molecular weight 200 kD) was reacted as described above in example 1.

¹H NMR (400 MHz, D₂O) δ 4.45 (bs, 2H), 4.0-3.1 (m, 18H), 1.95 (s, 3H).

Elem. Anal: C: 39.94%: H: 5.53%; N: 5.73%.

In the rest of the examples, a 700 kDa and 200 kDa hyaluronic acid, functionalized with X % of the azide linker is labeled 700 kDa [HA-N₃]-X % and 200 kDa [HA-N₃]-X % respectively.

[HA-N₃] intermediates may also be purified by tangential flow filtration. The reaction mixture diluted with 25 mL of 0.25 M NaCl solution and purified by tangential flow filtration. Tangential flow filtration was carried out using a 30 kDa MWCO Vivaflow-50R Hydrosart cartridge (Sartorius), eluting with 400 mL of 0.25 M NaCl solution, then 400 mL of water. The product was flash frozen and lyophilized to get final product.

### Example 2: Synthesis of Crosslinkers (XL)

### XL-1 Synthesis

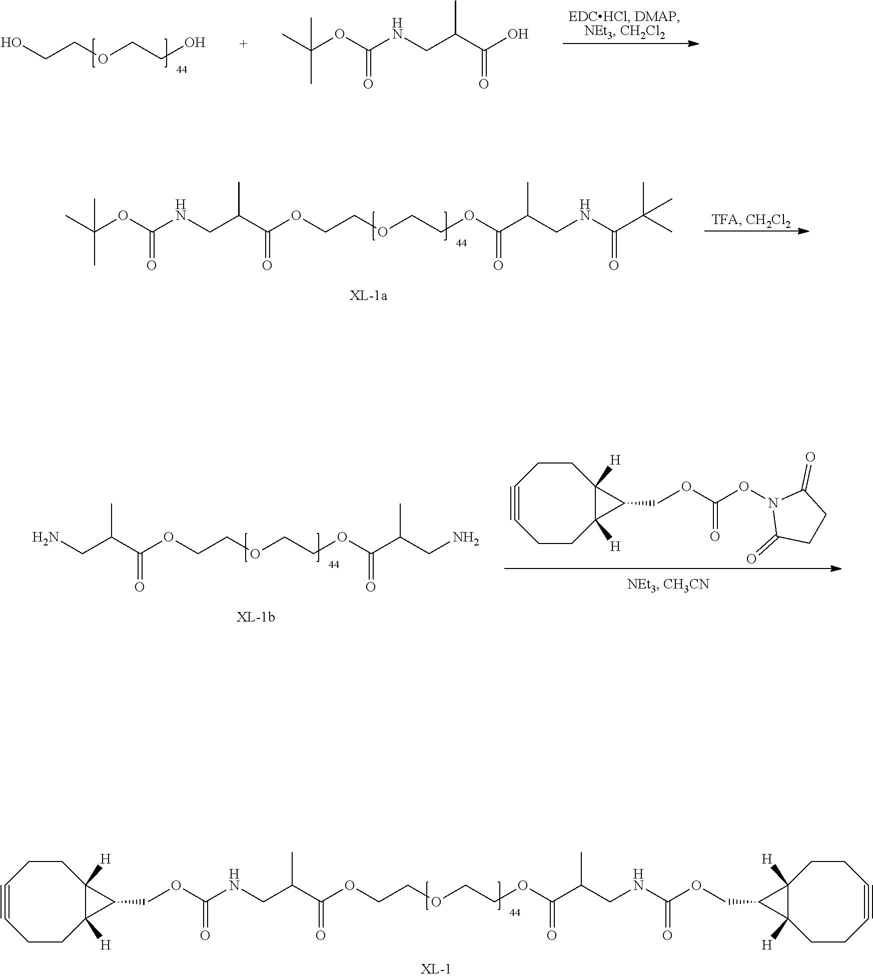

### XL-1a. PEG(2000)-bis-3-((tert-butoxycarbonyl)amino)-2-methylpropanoate

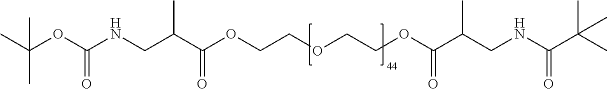

3-((tert-butoxycarbonyl)amino)propanoic acid (0.152 g, 0.75 mmol) and Mn ˜2 kDa PEG (0.5 g, 0.250 mmol) was dissolved in 15 mL dichloromethane. Dimethylaminopyridine (0.015 g, 0.125 mmol) and EDC HCl (0.192 g, 1.003 mmol) were added and the reaction mixture was stirred at room temperature overnight. The crude product was purified by flash column chromatography on silica with a 0-15% dichloromethane:methanol gradient. The product containing fractions were combined and reduced to dryness to provide XL-1a. ¹H NMR (400 MHz, methanol-d4) δ 4.23 (m, 4H), 3.63 (m, 170H), 3.22 (m, 4H) 2.66 (m, 2H), 1.43 (s, 18H), 1.13 (m, 6H).

### XL-1b. PEG (2000)-bis-[methyl-3-amino-2-methylpropanoate], bis-trifluoroacetic acid

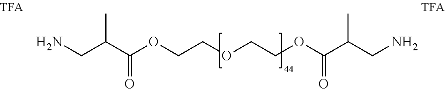

XL-1a (260 mg, 0.108 mmol) was dissolved in dichloromethane (3 mL). Trifluoroacetic acid was added (0.415 mL) and the reaction mixture was stirred at room temperature for 4h. The solvent was removed under reduced pressure. The crude product was triturated with Et₂O twice, then dried under vacuum to provide XL-1b. ¹H NMR (400 MHz, Methanol-d4) δ 4.45 (m, 2H), 4.22 (m, 2H), 3.59 (m, 177H), 3.12 (m, 4H) 2.87 (m, 2H), 1.22 (m, 6H).

### XL-1. PEG (2000)-bis 3-(((((1R,8S,9s)-bicyclo[6.1.0]non-4-yn-9-yl)methoxy)carbonyl)amino)-2-methylpropanoate

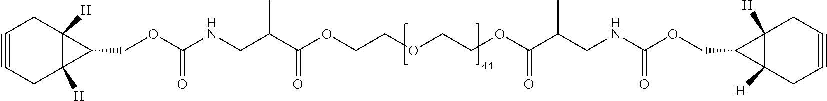

XL-1b (200 mg, 0.086 mmol) was dissolved in acetonitrile (3 mL). Triethylamine (0.599 mL, 4.30 mmol) was added, followed by ((1R,8S,9s)-bicyclo[6.1.0]non-4-yn-9-yl)methyl (2,5-dioxopyrrolidin-1-yl) carbonate (200 mg, 0.688 mmol) and the reaction mixture was stirred at room temperature for 4 h. The reaction mixture was directly purified by preparative reverse phase HPLC with ELSD triggered fraction collection (method below). The product containing fractions were pooled, frozen and lyophilized to provide XL-1. For storage purposes, XL-1 was kept as an acetonitrile, DMSO, or methanol solution in a freezer. Analytical HPLC-CAD (method below): retention time=2.75 min. 1H NMR (400 MHz, methanol-d4) δ 4.23 (m, 4H), 4.14 (m, 4H), 3.63 (m, 188H), 2.68 (m, 2H), 2.22 (m, 12H), 1.60 (m, 4H), 1.37 (m, 2H), 1.14 (m, 6H), 0.94 (m, 4H).

### XL-2 Synthesis

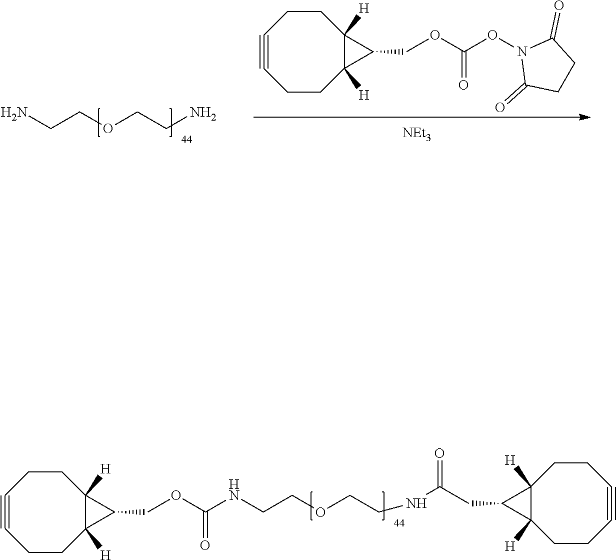

Mn ˜ 2 kDa PEG diamine hydrochloride (JenKem Technology, 300 mg, 0.148 mmol) was dissolved in acetonitrile (3 mL). Triethylamine (0.413 mL, 2.96 mmol) was added, followed by ((1R,8S,9s)-bicyclo[6.1.0]non-4-yn-9-yl)methyl (2,5-dioxopyrrolidin-1-yl) carbonate (345 mg, 1.184 mmol) and the reaction mixture was stirred at room temperature for 4 h. The reaction mixture was directly purified by preparative reverse phase HPLC with ELSD triggered fraction collection (method below). The product containing fractions were pooled, frozen and lyophilized to provide XL-2. For storage purposes, XL-2 was kept as an acetonitrile, DMSO, or methanol solution in a freezer. Analytical HPLC-CAD (method below): retention time=2.61 min. 1H NMR (400 MHz, methanol-d4) δ 4.14 (m, 4H), 3.63 (br s, 186H), 3.54 (m, 4H), 2.22 15 (m, 12H), 1.61 (m, 4H), 1.38 (m, 2H), 0.94 (m, 4H).

Preparative HPLC conditions: Waters XBridge C18; particle size: 5 m; column size: 19×250 mm; eluent/gradient: 5% CH₃CN/H₂O/0.5 min, 5-95% CH3CN/H₂O/12.5 min, 95% CH₃CN/H₂O/3 min; flow rate: 30 mL/min; column temperature: room temperature.

Analytical HPLC-CAD conditions: Waters ACQUITY UPLC BEH C18; particle size: 1.7 μm; column size: 2.1×50 mm; eluent/gradient: 2% CH₃CN/H₂O/0.5 min, 2-98% CH₃CN/H₂O/5 min (CH₃CN containing 0.1% formic acid and H₂O containing 0.1% formic acid); flow rate: 1 mL/min; column temperature: 50° C.

### Example 3: Preparation of HA-Hydrogel Formulations for In Vitro and In Vivo Studies

The crosslinker solution (50 mg/mL) prepared in Example 2 was dried under reduced pressure to remove the ACN (acetonitrile). The same amount of 1×PBS was added to the dry residue to get 50 mg/mL concentration of crosslinker in 1×PBS buffer. This freshly prepared solution was used for preparation of formulations for in vitro and in vivo studies.

H1a. In Situ Forming HA-Hydrogel Synthesis

200 kDa [HA-N₃]-24% (122.4 mg, degree of substitution=24%) was dissolved in 7.26 mL 1×PBS buffer pH 7.4 (16.9 mg/mL, as weighed concentration) and stirred at room temperature overnight while protected from light. The molecular weight of the unsubstituted carboxylate sodium salt repeat dimer unit is 401.3 Da. The MW of the azidylated repeat dimer unit is 579.6 Da. The average MW of a dimer unit for the sodium salt form of 200 kDa [HA-N₃]-24% is 444.1 Da=((401.3×0.76)+(579.6×0.24)). Using the average MW of a sodium salt dimer unit, the total moles of repeat dimer unit is 276 μmol and the number of moles of azidylated repeat dimer unit is 66.2 μmol. 200 μL of 200 kDa HA-N₃-24% (3.38 mg, 7.61 μmol) was aliquoted from a stock solution and gelation was carried out by adding 10.4 μl of XL-2 crosslinker (0.52 mg, 0.221 μmol of reagent, 0.442 μmol of reactive functionality, 50 mg/mL in 1×PBS). This resulted in a solution with predicted crosslinking of 6.7% of the 200 kDa [HA-N3]-24% repeat units by the XL-2 ((0.442 μmol [XL-2-reactive functionality]/66.2 μmol [HAunits)×100=6.7%) The mixture was vortexed and 50 μl aliquot of the mixed solution was quickly added to an Eppendorf tube and stored at room temperature for overnight. The next day, visual inspection showed formation of Ha gel.

### H2a Hydrogel Synthesis

VEGF-C protein was mixed with 200 kDa [HA-N₃]-24% solution and a gelation reaction was performed with the crosslinker XL-2 as described above to prepare H2a gel.

### H3a Hydrogel Synthesis

VEGF-C protein was mixed with 200 kDa [HA-N₃]-24% solution and a gelation reaction was performed with the 2×amount of crosslinker XL-1 as described above to prepare H3a gel.

### H4a Hydrogel Synthesis

VEGF-C protein was mixed with 700 kDa [HA-N₃]-16% solution and a gelation reaction was performed with the 2×amount of crosslinker XL-2 as described above to prepare H4a gel.

### H5a Hydrogel Synthesis

VEGF-C protein was complexed with laponite XLG (BYK additives) prior to addition to 700 kDa [HA-N₃]-16% solution with laponite conc. 0.25 mg/mL in final gel. A gelation reaction was performed with the 2×amount of crosslinker XL-2 as described above to prepare H5a gel.

### H6a Hydrogel Synthesis

VEGF-C protein was complexed with laponite XLG (BYK additives) prior to addition to 700 kDa [HA-N₃]-16% solution with laponite conc. 1 mg/mL in final gel. A gelation reaction was performed with the 2×amount of crosslinker XL-2 as described above to prepare H6a gel.

H3b. HA Hydrogel Particles Synthesis 200 μL of H3a gel prepared as described above was forced through a 100 mesh stainless steel screen disc into a 1 mL syringe, yielding coarse gel particles. 100 μL of 1×PBS was added to this syringe, followed by vortexing to mix. The syringe was held at room temperature for 6 h to allow swelling of the hydrogel. The swollen, coarse gel particles of H3a were forced 20 times through a 200-mesh stainless steel screen disc, yielding fine gel particles to get H3b product.

### H5b Hydrogel Synthesis

H5a gel was extruded to fine gel particles to get final H5b product.

### H6b Hydrogel Synthesis

H6a gel was extruded to fine gel particles to get final H6b product.

### In Vitro Release Study Set Up

950 μl PBS-2% BSA buffer was added to the 50 μL aliquot of hydrogel and incubated at 37° C. on 300 rpm shaking. 800 μl aliquots were taken out at different time points and no gel was removed at those time points. The study samples were replenished with same amount of 1×PBS-2% BSA buffer to keep the volume of study sample same. The release samples were then analyzed with ELISA for VEGF-C content.

### In Vivo Study Set Up

After all the components for preparation of H3a gel was mixed, the tube was vortexed and quickly filled in the back of insulin syringe. The sample was pushed inside the syringe with a plunger and any air bubbles formed was removed. 30 μL was injected intradermally in mice within 2-3 minutes of mixing.

### Results

Sustained release of VEGF-C was achieved in vitro using different compositions of HA-hydrogels (FIGS. 54A-54D). No difference was observed in the release curves for HA-hydrogels prepared with 700 kDa [HA-N₃]-16% and 200 kDa [HA-N₃]-24% (FIG. 54A). The VEGF-C release from HA-hydrogel formulations can be tuned by the addition of laponite. The laponite containing HA-hydrogel formulations provided concentration-dependent slower release of VEGF-C protein as compared to the formulations containing no laponite (FIG. 54B). The laponite containing in situ forming hydrogel and HA-hydrogel particles provided a similar release profile for VEGF-C protein (FIGS. 54C and 54D).

The in vivo study was performed with intradermal injection of H1a and H3a in situ forming hydrogels in mice. The VEGF-C containing formulation (H3a) showed an increase in lymphatic endothelial cells (LECs) and T4 cells after day 7 analysis as compared to the control (H1a) formulation (FIGS. 55A and 55B).

### EQUIVALENTS

The disclosures of each and every patent, patent application, and publication cited herein are hereby incorporated herein by reference in their entirety. While this invention has been disclosed with reference to certain embodiments, it is apparent that further embodiments and variations of this invention may be devised by others skilled in the art without departing from the true spirit and scope of the invention. The appended claims are intended to be construed to include all such embodiments and equivalent variations.

What is claimed is:

1. A plurality of compositions, comprising:
a first composition comprising:
a biomaterial and a cell recruitment factor; and
a second composition comprising:
a viral vector.

2. A first composition, comprising:
a biomaterial and a cell recruitment factor,
wherein the biomaterial comprises a hydrogel, e.g., a cryogel (e.g., an alginate cryogel) or a hyaluronic acid hydrogel (HA hydrogel), and
wherein the cell recruitment factor comprises an amino acid sequence according to SEQ ID NO: 741, or an amino acid sequence with at least 80%, 85%, 90%, 95%, or 99% sequence identity thereto, provided that the amino acid at position 26 of SEQ ID NO: 741 is not Cysteine (C), optionally wherein the amino acid at position 26 of SEQ ID NO: 741 is Alanine (A).

3. A method of transducing cells of a subject in vivo or treating a disease, disorder, or condition in a subject, the method comprising:
administering a biomaterial and a cell recruitment factor to a site (e.g., high subcutaneous space or subcutaneous space adjacent to dermis) in the subject, and
administering a viral vector or a nucleic acid comprising a transgene to the subject; thereby transducing cells of the subject with the transgene.

4. The method of claim 3, wherein the biomaterial and the cell recruitment factor are comprised in a first composition, and the viral vector or nucleic acid is comprised in a second composition.

5. The plurality of compositions of claim 1, or the method of any one of claims 3-4, wherein the biomaterial:
(i) comprises a hydrogel;
(ii) comprises a cryogel, e.g., an alginate cryogel;
(iii) comprises a hyaluronic acid hydrogel (HA hydrogel);
(iv) comprises a gelatin, hyaluronic acid, collagen, alginate, laminin, chitosan, silk fibroin, agarose, poly(ethylene glycol), poly-vinyl alcohol, and/or hydroxyethyl methylacrylate;
(v) comprises alginate hydrogel, optionally wherein the alginate hydrogel further comprises norbornene and/or tetrazine, optionally wherein the norbornene and/or tetrazine is covalently associated with, e.g., chemically linked to, or non-covalently associated with, e.g., adsorbed on, the alginate; and/or
(vi) comprises pores between about 10 μm to about 300 μm, e.g., between about 50 μm to about 300 μm, in diameter, or no pores; and/or
(vii) is chemically crosslinked.

6. The plurality of compositions, the first composition, or the method of any one of the preceding claims, wherein the first composition comprising the biomaterial further comprises laponite; or wherein the biomaterial is comprised within a first composition that further comprises laponite; optionally wherein the laponite is present at a concentration of about 0.15 mg/mL to about 0.35 mg/mL, e.g., about 0.25 mg/mL.

7. The plurality of compositions, the first composition, or the method of any one of the preceding claims, wherein the cell recruitment factor is:
(i) noncovalently associated with, e.g., adsorbed on, the biomaterial; or
(ii) covalently associated with, e.g., conjugated to, the biomaterial.

8. The plurality of compositions, the first composition, or the method of any one of the preceding claims, wherein the cell recruitment factor:
(i) induces lymphangiogenesis;
(ii) induce growth of lymphatic endothelial cells; and/or
(ii) recruits immune cells, optionally wherein the immune cells comprise T-cells and/or NK-cells.

9. The plurality of compositions, the first composition, or the method of claim 8, wherein induction of lymphangiogenesis:
(i) comprises an increase in the level of lymphatic endothelial cells (LECs) (e.g., CD45−CD31+PDPN+ cells), optionally wherein the level of LECs is increased by at least 10%, 20%, 30%, 40%, 50%, 60, 70%, 75%, 80%, 85%, 90%, 95%, 100%, or 200% as compared to a reference level (e.g., the level of LECs at a site in a subject prior to injection of the plurality of compositions or the first composition), when measured by an assay, e.g., a flow cytometry assay, e.g., as described in Example H or I; and/or
(ii) results in at least 50 LECs (e.g., at least 75, 100, 125, 150, 200, 225, or 250 LECs) per milligram of tissue when measured by an assay, e.g., a flow cytometry assay, e.g., as described in Example H or I.

10. The plurality of compositions, the first composition, or the method of any one of the preceding claims, wherein the cell recruitment factor recruits T cells, optionally wherein the T cells comprise naïve T cells (e.g., CD45RA+CD62L+ T cells or CD45RA+CD62L+CCR7+CD27+CD95+ T cells).

11. The plurality of compositions, the first composition, or the method of claim 10, wherein recruitment of T cells comprises an increase in the level of T cells, optionally wherein the level of T cells is increased by at least 10%, 20%, 30%, 40%, 50%, 60, 70%, 75%, 80%, 85%, 90%, 95%, 100%, 200%, or 300% as compared to a reference level (e.g., the level of T cells at a site in a subject prior to injection of the plurality of compositions or the first composition), when measured by an assay, e.g., a flow cytometry assay, e.g., as described in Example H or I.

12. The plurality of compositions of any one of claims 1 or 5-11, or the method of any one of claims 3-11, wherein the cell recruitment factor is chosen from VEGF-C, IL-2, IL-7, IL-15 (e.g., hetIL-15 (IL15/sIL-15Ra)), GM-CSF, CXCL12, CXC3L1, CCL19, CCL21, CXCL10, or CXCL11.

13. The plurality of compositions of any one of claims 1 or 5-12, or the method of any one of claims 3-12, wherein the cell recruitment factor comprises VEGF-C, optionally wherein the VEGF-C:
(i) comprises a mature VEGF-C peptide, optionally the minor or major mature form or a mutated variant thereof,
(ii) is a monomer or dimer; and/or
(iii) is present in an effective amount, optionally, in an amount of less than or about 1 mg, less than or about 10 mg, greater than or about 10 μg, greater than or about 1 μg, between about 1 μg and 1 mg, between about 10 μg and 1 mg, between about 1 μg and 10 mg, or between about 10 μg and 10 mg.

14. The plurality of compositions or the method of claim 13, wherein the VEGF-C comprises:
(i) an amino acid sequence of any one of the sequences provided in Table 18 or a sequence with at least 95% sequence identity thereto, optionally wherein the sequence comprises or does not comprise a linker (e.g., a glycine-serine linker) and/or a his tag; and/or
(ii) the amino acid substitution of C137A, numbered according to SEQ ID NO: 725.

15. The plurality of compositions of any one of claims 1 or 5-14 or the method of any one of claims 3-14, wherein the cell recruitment factor comprises:
(i) an amino acid sequence according to SEQ ID NO: 741, or an amino acid sequence with at least 80%, 85%, 90%, 95%, or 99% sequence identity thereto, provided that the amino acid at position 26 of SEQ ID NO: 741 is not Cysteine (C), optionally wherein the amino acid at position 26 of SEQ ID NO: 741 is Alanine (A);
(ii) the amino acid sequence according to SEQ ID NO: 743 or a sequence an amino acid sequence with at least 80%, 85%, 90%, 95%, or 99% sequence identity thereto;
(iii) the amino acid sequence according to SEQ ID NO: 740 or an amino acid sequence with at least 80%, 85%, 90%, 95%, or 99% sequence identity thereto;
(iv) the amino acid sequence according to SEQ ID NO: 736, or an amino acid sequence with at least 80%, 85%, 90%, 95%, or 99% sequence identity thereto;
(v) a linker, e.g., wherein the linker has a sequence of Gly-Ser, wherein optionally the linker is C-terminal of SEQ ID NO: 743 or a sequence having at least 80%, 85%, 90%, 95%, or 99% sequence identity thereto;
(vi) the amino acid sequence according to SEQ ID NO: 735, or an amino acid sequence with at least 80%, 85%, 90%, 95%, or 99% sequence identity thereto;
(vii) the amino acid sequence according to SEQ ID NO: 734, or an amino acid sequence with at least 80%, 85%, 90%, 95%, or 99% sequence identity thereto; and/or
(viii) the amino acid sequence according to SEQ ID NO: 733, or an amino acid sequence with at least 80%, 85%, 90%, 95%, or 99% sequence identity thereto.

16. The plurality of compositions of any one of claims 1 or 5-15 or the method of any one of claims 3-15, wherein the cell recruitment factor comprises VEGF-C or a functional variant thereof, IL-15 (e.g., hetIL-15 (IL15/sIL-15Ra)) or a functional variant thereof; TL-7 or a functional variant thereof; or a combination thereof.

17. The plurality of compositions of any one of claims 1 or 5-16, or the method of any one of claims 4-16, wherein the second composition further comprises a particle.

18. The plurality of compositions or the method of claim 17, wherein the particle is a mesoporous particle, a silica particle and/or a mesoporous silica particle, optionally wherein the mesoporous silica particle is a mesoporous silica rod.

19. A second composition comprising:
a mesoporous silica particle;
a viral vector; and
a cell activation agent.

20. The plurality of compositions or the method of claim 18, or the second composition of claim 19, wherein the mesoporous silica particle:
(i) comprises a surface modification, optionally wherein the surface modification comprises:
(a) a —OH (hydroxyl), amine, carboxylic acid, phosphonate, halide, azide, alkyne, epoxide, sulfhydryl, polyethyleneimine, a hydrophobic moiety, or salts thereof, optionally using a C₁ to C₂₀ alkyl or (—O(CH2-CH₂—)₁₋₂₅ linker;
(b) a primary, secondary, tertiary, or quaternary amine; and/or
(c) a polyethyleneimine having an average molecular weight of about 1000 to 20,000 Da, about 1,200 to 15,000 Da, about 1,500 to 12,000 Da, about 2,000 Da, about 3,000 Da, about 4,000 Da, about 5,000 Da, about 6,000 Da, about 7,000 Da, about 8,000 Da, about 9,000 Da, or about 10,000 Da, as measured by gel permeation chromatography (GPC);
(ii) is a trimethylammonium functionalized mesoporous silica particle, e.g., a N,N,N-trimethylpropan-1-ammonium functionalized mesoporous silica particle;
(iii) comprises a plurality of pores, optionally wherein the pores are between 2-50 nm in diameter; and/or
(iv) comprises a surface area of at least about 100 m²/g.

21. The plurality of compositions of any one of claims 1, 5-18, or 20, the second composition of claim 19 or 20, or the method of any one of claims 3-18 or 20, wherein:
(i) the viral vector is noncovalently, e.g., electrostatically, or covalently associated with the mesoporous silica particle; and/or
(ii) the cell activation agent is noncovalently or covalently associated with the mesoporous silica particle.

22. A method of transducing cells of a subject in vivo or treating a disease, disorder, or condition in a subject, the method comprising:
administering a viral vector or a nucleic acid comprising a transgene to a site in the subject,
wherein the subject has previously been administered a biomaterial and a cell recruitment factor in an amount sufficient to induce lymphangiogenesis and/or recruitment of T cells to the site in the subject;
thereby transducing the cells.

23. The method of claim 22, wherein the viral vector is noncovalently, e.g., electrostatically, or covalently associated with a particle, e.g., a mesoporous silica particle.

24. The plurality of compositions of any one of claims 1, 5-18, or 20-21, the second composition of any one of claims 19-21, or the method of any one of claims 3-18, or 20-23, wherein the viral vector comprises:
(i) a lentivirus, retrovirus, adenovirus, adeno-associated virus, or herpes virus; and/or
(ii) an expression vector comprising a recombinant polynucleotide comprising an expression control sequence operatively linked to a nucleotide sequence to be expressed.

25. The plurality of compositions, the second composition, or the method of claim 24, wherein the nucleotide sequence encodes: a chimeric antigen receptor (CAR), an engineered TCR, a cytokine, a chemokine, an shRNA, or a polypeptide engineered to target a tumor antigen.

26. The plurality of compositions, the second composition, or the method of claim 25, wherein the tumor antigen is selected from the group consisting of: TSHR, CD19, CD123, CD22, CD30, CD171, CS-1, CLL-1, CD33, EGFRvIII, GD2, GD3, BCMA, Tn Ag, PSMA, ROR1, FLT3, FAP, TAG72, CD38, CD44v6, CEA, EPCAM, B7H3, KIT, IL-13Ra2, Mesothelin, IL-11 Ra, PSCA, PRSS21, VEGFR2, LewisY, CD24, PDGFR-beta, SSEA-4, CD20, Folate receptor alpha, ERBB2 (Her2/neu), MUC1, EGFR, NCAM, Prostase, PAP, ELF2M, Ephrin B2, IGF-I receptor, CAIX, LMP2, gp100, bcr-abl, tyrosinase, EphA2, Fucosyl GM1, sLe, GM3, TGS5, HMWMAA, o-acetyl-GD2, Folate receptor beta, TEM1/CD248, TEM7R, CLDN6, GPRC5D, CXORF61, CD97, CD179a, ALK, Polysialic acid, PLAC1, GloboH, NY-BR-1, UPK2, HAVCR1, ADRB3, PANX3, GPR20, LY6K, OR51E2, TARP, WT1, NY-ESO-1, LAGE-1a, MAGE-A1, legumain, HPV E6,E7, MAGE A1, ETV6-AML, sperm protein 17, XAGE1, Tie 2, MAD-CT-1, MAD-CT-2, Fos-related antigen 1, p53, p53 mutant, prostein, survivin and telomerase, PCTA-1/Galectin 8, MelanA/MART1, Ras mutant, hTERT, sarcoma translocation breakpoints, ML-IAP, ERG (TMPRSS2 ETS fusion gene), NA17, PAX3, Androgen receptor, Cyclin B1, MYCN, RhoC, TRP-2, CYP1B1, BORIS, SART3, PAX5, OY-TES1, LCK, AKAP-4, SSX2, RAGE-1, human telomerase reverse transcriptase, RU1, RU2, intestinal carboxyl esterase, mut hsp70-2, CD79a, CD79b, CD72, LAIR1, FCAR, LILRA2, CD300LF, CLEC12A, BST2, EMR2, LY75, GPC3, FCRL5, IGLL1, and any combination thereof.

27. The plurality of compositions of any one of claims 1, 5-18, 20-21, or 24-26, the second composition of any one of claims 19-21 or 24-26, or the method of any one of claims 3-18 or 20-26, wherein the viral vector encodes a CAR that comprises an antigen binding domain, a transmembrane domain, a costimulatory signaling region, and a signaling domain, wherein:
(i) the antigen binding domain binds an antigen selected from the group consisting of CD19, CD123, CD22, CD20, EGFRvIII, BCMA, Mesothelin, CD33, CLL-1, and any combination thereof,
(ii) the transmembrane domain comprises a CD8 hinge;
(iii) the costimulatory signaling region is selected from a 4-1BB or CD28 costimulatory signaling domain; and/or
(iv) the signaling domain comprises a CD3 zeta signaling domain.

28. The plurality of compositions of any one of claims 1, 5-18, 20-21, or 24-27, or the method of any one of claims 4-18 or 20-27, wherein the second composition further comprises a cell activation agent.

29. The plurality of compositions or the method of claim 28, the second composition of any one of claims 19-21 or 24-27, wherein the cell activation agent:
(a) comprises an agent that stimulates CD3/TCR complex and/or an agent that stimulates a costimulatory molecule and/or growth factor receptor;
(b) is a multispecific binding molecule comprising: (i) an anti-CD3 binding domain, and (ii) a costimulatory molecule binding domain (e.g., an anti-CD2 binding domain or an anti-CD28 binding domain);
(c) the amino acid sequence of any heavy chain provided in Table 20, or an amino acid sequence with at least 95% sequence identity thereto; and/or the amino acid sequence of any light chain provided in Table 20, or an amino acid sequence with at least 95% sequence identity thereto; and/or
(d) is conjugated to or adsorbed on the particle, e.g., mesoporous silica particle.

30. The plurality of compositions, the method, or the second composition of claim 29, wherein:
(i) the anti-CD3 binding domain, e.g., an anti-CD3 scFv, is situated N-terminal of the costimulatory molecule binding domain, e.g., an anti-CD2 Fab or an anti-CD28 Fab; or
(ii) the anti-CD3 binding domain, e.g., an anti-CD3 scFv, is situated C-terminal of the costimulatory molecule binding domain, e.g., an anti-CD2 Fab or an anti-CD28 Fab, optionally wherein:
an Fc region is situated between the anti-CD3 binding domain and the costimulatory molecule binding domain; or
the multispecific binding molecule comprises a CH2, and the anti-CD3 binding domain is situated N-terminal of the CH2.

31. The plurality of compositions, the method, or the second composition of claim 29 or 30, wherein the multispecific binding molecule comprises:
(i) a first polypeptide comprising from N-terminal to C-terminal: VH of the anti-CD3 binding domain, VL of the anti-CD3 binding domain, VH of the costimulatory molecule binding domain, CH1, CH2, and CH3; and
(ii) a second polypeptide comprising from N-terminal to C-terminal: VL of the costimulatory molecule binding domain and CL.

32. The plurality of compositions, the method, or the second composition of claim 29 or 30, wherein the multispecific binding molecule comprises:
(i) a first polypeptide comprising from N-terminal to C-terminal: VH of the costimulatory molecule binding domain, CH1, CH2, CH3, VH of the anti-CD3 binding domain, and VL of the anti-CD3 binding domain; and
(ii) a second polypeptide comprising from N-terminal to C-terminal: VL of the costimulatory molecule binding domain and CL.

33. The plurality of compositions, the method, or the second composition of claim 29 or 30, wherein the multispecific binding molecule comprises:
(i) a first polypeptide comprising from N-terminal to C-terminal: VH of the costimulatory molecule binding domain, CH1, VH of the anti-CD3 binding domain, VL of the anti-CD3 binding domain, CH2, and CH3; and
(ii) a second polypeptide comprising from N-terminal to C-terminal: VL of the costimulatory molecule binding domain and CL.

34. The plurality of compositions, the method, or the second composition of claim 29 or 30, wherein the multispecific binding molecule comprises an Fc region comprising:
(i) a L234A, L235A, S267K, and P329A mutation (LALASKPA), numbered according to the Eu numbering system;
(ii) a L234A, L235A, and P329G mutation (LALAPG), numbered according to the Eu numbering system;
(iii) a G237A, D265A, P329A, and S267K mutation (GADAPASK), numbered according to the Eu numbering system;
(iv) a L234A, L235A, and G237A mutation (LALAGA), numbered according to the Eu numbering system;
(v) a D265A, P329A, and S267K mutation (DAPASK), numbered according to the Eu numbering system;
(vi) a G237A, D265A, and P329A mutation (GADAPA), numbered according to the Eu numbering system;
(vii) a L234A, L235A, and P329A mutation (LALAPA), numbered according to the Eu numbering system; or
(viii) an amino acid sequence of any of the Fc regions in Table 20 or an amino acid sequence having at least 95% identity thereto.

35. The plurality of compositions, the method, or the second composition of claim 29, 30, or 34, wherein the multispecific binding molecule comprises:
(i) a heavy chain comprising the amino acid sequence of any of SEQ ID NOs: 726, 893, or 895, or an amino acid sequence having at least 95% sequence identity thereto; and/or
(ii) a light chain comprising the amino acid sequence of any of SEQ ID NOs: 728, 730, 892, or 894, or an amino acid sequence having at least 95% sequence identity thereto.

36. The plurality of compositions, the method, or the second composition of any one of claims 29, 30, or 34-35, wherein the multispecific binding molecule comprises:
(i) a heavy chain comprising the amino acid sequence of SEQ ID NO: 726 or an amino acid sequence having at least 95% sequence identity thereto, and a light chain comprising the amino acid sequence of SEQ ID NO: 728, or an amino acid sequence having at least 95% sequence identity thereto;
(ii) a heavy chain comprising the amino acid sequence of SEQ ID NO: 726 or an amino acid sequence having at least 95% sequence identity thereto, and a light chain comprising the amino acid sequence of SEQ ID NO: 730, or an amino acid sequence having at least 95% sequence identity thereto;
(iii) a heavy chain comprising the amino acid sequence of SEQ ID NO: 1416 or an amino acid sequence having at least 95% sequence identity thereto, and a light chain comprising the amino acid sequence of SEQ ID NO: 728, or an amino acid sequence having at least 95% sequence identity thereto;
(iv) a heavy chain comprising the amino acid sequence of SEQ ID NO: 1416 or an amino acid sequence having at least 95% sequence identity thereto, and a light chain comprising the amino acid sequence of SEQ ID NO: 730, or an amino acid sequence having at least 95% sequence identity thereto;
(v) a heavy chain comprising the amino acid sequence of SEQ ID NO: 893 or an amino acid sequence having at least 95% sequence identity thereto, and a light chain comprising the amino acid sequence of SEQ ID NO: 892, or an amino acid sequence having at least 95% sequence identity thereto;
(vi) a heavy chain comprising the amino acid sequence of SEQ ID NO: 1417 or an amino acid sequence having at least 95% sequence identity thereto, and a light chain comprising the amino acid sequence of SEQ ID NO: 892, or an amino acid sequence having at least 95% sequence identity thereto; or
(vii) a heavy chain comprising the amino acid sequence of SEQ ID NO: 895 or an amino acid sequence having at least 95% sequence identity thereto, and a light chain comprising the amino acid sequence of SEQ ID NO: 894, or an amino acid sequence having at least 95% sequence identity thereto.

37. The plurality of compositions of any one of claims 1, 5-18, 20-21, or 24-36, or the method of any one of claims 4-18 or 20-36, wherein the second composition further comprises a first population of particles and a second population of particles, e.g., a first population of mesoporous silica particles and a second population of mesoporous silica particles, wherein the first population comprises the viral vector and the second population comprises a cell activation agent, e.g., wherein the viral vector is noncovalently associated with a particle of the first population and the cell activation agent is noncovalently associated with a particle of the second population.

38. The plurality of compositions of any one of claims 1, 5-18, 20-21, or 24-37, the first composition of any one of claims 2, or 6-11, or the second composition of any one of claims 19-21, 24-27, or 29-36, wherein the first or second composition is suitable for injectable use.

39. The plurality of compositions of any one of claims 1, 5-18, 20-21, or 24-38, the first composition of any one of claims 2, 6-11, or 38, or the second composition of any one of claims 19-21, 24-27, 29-36, or 38, which further comprises:
(i) a Tet2 inhibitor, optionally wherein the Tet2 inhibitor comprises: (1) a gene editing system targeted to one or more sites within the gene encoding Tet2, or its corresponding regulatory elements; (2) a nucleic acid (e.g., an siRNA or shRNA) that inhibits expression of Tet2; (3) a protein (e.g., a dominant negative, e.g., catalytically inactive) Tet2, or a binding partner of Tet2 (e.g., a dominant negative binding partner of Tet2); (4) a small molecule that inhibits expression and/or function of Tet2; (5) a nucleic acid encoding any of (1)-(3); or (6) any combination of (1)-(5); and/or
(ii) a ZBTB32 inhibitor, optionally wherein the ZBTB32 inhibitor comprises: (1) a gene editing system targeting the ZBTB32 gene or one or more components thereof, (2) a nucleic acid encoding one or more components of the gene editing system; or (3) a combination of (1) and (2). In an embodiment, the ZBTB32 inhibitor comprises: (1) a gene editing system targeting the ZBTB32 gene or one or more components thereof. In an embodiment, the ZBTB32 inhibitor comprises (2) a nucleic acid encoding one or more components of the gene editing system. In an embodiment, the ZBTB32 inhibitor comprises a combination of (1) and (2).

40. The method of any one of claims 3-18 or 20-37, which further comprises administering to the subject:
(i) a Tet2 inhibitor, optionally wherein the Tet2 inhibitor comprises: (1) a gene editing system targeted to one or more sites within the gene encoding Tet2, or its corresponding regulatory elements; (2) a nucleic acid (e.g., an siRNA or shRNA) that inhibits expression of Tet2; (3) a protein (e.g., a dominant negative, e.g., catalytically inactive) Tet2, or a binding partner of Tet2 (e.g., a dominant negative binding partner of Tet2); (4) a small molecule that inhibits expression and/or function of Tet2; (5) a nucleic acid encoding any of (1)-(3); or (6) any combination of (1)-(5); and/or
(ii) a ZBTB32 inhibitor, optionally wherein the ZBTB32 inhibitor comprises: (1) a gene editing system targeting the ZBTB32 gene or one or more components thereof, (2) a nucleic acid encoding one or more components of the gene editing system; or (3) a combination of (1) and (2). In an embodiment, the ZBTB32 inhibitor comprises: (1) a gene editing system targeting the ZBTB32 gene or one or more components thereof. In an embodiment, the ZBTB32 inhibitor comprises (2) a nucleic acid encoding one or more components of the gene editing system. In an embodiment, the ZBTB32 inhibitor comprises a combination of (1) and (2).

41. A method of transducing cells of a subject in vivo or treating a disease, disorder, or condition in a subject, comprising administering the first composition and the second composition of the plurality of compositions of any one of claims 1, 5-18, 20-21, or 24-39.

42. A method of transducing cells of a subject in vivo or treating a disease, disorder, or condition in a subject, comprising administering the first composition of any one of claims 2, 6-11, 38, or 39, and the second composition of any one of claims 19-21, 24-27, 29-36, or 38.

43. The method of any one of claims 3-18, 20-21, 24-37, or 40-42, wherein the first composition and the second composition are administered sequentially.

44. The method of any one of claims 3-18, 20-21, 24-37, or 40-43, wherein the first composition is administered prior to the administration of the second composition, optionally wherein:
(i) the first composition is administered about 1-4 weeks, e.g., about 2 weeks, prior to the administration of the second composition; or
(ii) the first composition is administered at least two weeks prior to the administration of the second composition.

45. The method of any one of claims 3-18, 20-21, 24-37, or 40-44, further comprising evaluating, e.g., measuring, lymphangiogenesis in a sample from the subject (e.g., a sample from or close to the site of administration), wherein lymphangiogenesis is measured after the administration of the first composition and/or prior to the administration of the second composition,
optionally wherein measuring lymphangiogenesis comprises acquiring a value for the level and/or activity of lymphatic endothelial cells (LECs) (e.g., CD45−CD31+PDPN+ cells) in the sample.

46. The method of any one of claims 3-18, 20-21, 24-37, or 40-45, further comprising evaluating, e.g., measuring, the recruitment of T cells in a sample from the subject (e.g., a sample from or close to the site of administration), wherein the recruitment of T cells is measured after the administration of the first composition and/or prior to the administration of the second composition,
optionally wherein measuring the recruitment of T cells comprises acquiring a value for the level and/or activity of T cells (e.g., naïve T cells, e.g., CD45RA+CD62L+ T cells and/or CD45RA+CD62L+CCR7+CD27+CD95+ T cells) in the sample.

47. The plurality of compositions of any one of claims 1, 5-18, 20-21, or 24-39 for use in a method of transducing cells of a subject in vivo or treating a disease, disorder, or condition in a subject.

48. The first composition of any one of claims 2, 6-11, 38, or 39, in combination with the second composition of any one of claims 19-21, 24-27, 29-36, or 38 for use in a method of transducing cells of a subject in vivo or treating a disease, disorder, or condition in a subject.

49. The second composition of any one of claims 19-21, 24-27, 29-36, or 38, in combination with the first composition of any one of claims 2, 6-11, 38, or 39, for use in a method of transducing cells of a subject in vivo or treating a disease, disorder, or condition in a subject.

50. The method of any one of claims 3-18, 20-37, or 40-46, wherein:
(i) the subject has or has been diagnosed with having a disease, disorder, or condition; and/or
(ii) the subject is a human.

51. The method of any one of claims 3-18, 20-37, 40-46, or 50, wherein the disease, disorder, or condition comprises:
(i) a cancer;
(ii) a hematological cancer, optionally wherein the hematological cancer comprises a leukemia or lymphoma;
(iii) a hematological cancer chosen from chronic lymphocytic leukemia (CLL), mantle cell lymphoma (MCL), multiple myeloma, acute lymphoid leukemia (ALL), Hodgkin lymphoma, B-cell acute lymphoid leukemia (BALL), T-cell acute lymphoid leukemia (TALL), small lymphocytic leukemia (SLL), B cell prolymphocytic leukemia, blastic plasmacytoid dendritic cell neoplasm, Burkitt©lymphoma, diffuse large B cell lymphoma (DLBCL), DLBCL associated with chronic inflammation, chronic myeloid leukemia, myeloproliferative neoplasms, follicular lymphoma, pediatric follicular lymphoma, hairy cell leukemia, small cell- or a large cell-follicular lymphoma, malignant lymphoproliferative conditions, MALT lymphoma (extranodal marginal zone lymphoma of mucosa-associated lymphoid tissue), Marginal zone lymphoma, myelodysplasia, myelodysplastic syndrome, non-Hodgkin lymphoma, plasmablastic lymphoma, plasmacytoid dendritic cell neoplasm, Waldenstrom macroglobulinemia, splenic marginal zone lymphoma, splenic lymphoma/leukemia, splenic diffuse red pulp small B-cell lymphoma, hairy cell leukemia-variant, lymphoplasmacytic lymphoma, a heavy chain disease, plasma cell myeloma, solitary plasmocytoma of bone, extraosseous plasmocytoma, nodal marginal zone lymphoma, pediatric nodal marginal zone lymphoma, primary cutaneous follicle center lymphoma, lymphomatoid granulomatosis, primary mediastinal (thymic) large B-cell lymphoma, intravascular large B-cell lymphoma, ALK+ large B-cell lymphoma, large B-cell lymphoma arising in HHV8-associated multicentric Castleman disease, primary effusion lymphoma, B-cell lymphoma, acute myeloid leukemia (AML), or unclassifiable lymphoma;
(iv) a solid cancer; or
(v) a solid cancer chosen from: one or more of mesothelioma, malignant pleural mesothelioma, non-small cell lung cancer, small cell lung cancer, squamous cell lung cancer, large cell lung cancer, pancreatic cancer, pancreatic ductal adenocarcinoma, esophageal adenocarcinoma, breast cancer, glioblastoma, ovarian cancer, colorectal cancer, prostate cancer, cervical cancer, skin cancer, melanoma, renal cancer, liver cancer, brain cancer, thymoma, sarcoma, carcinoma, uterine cancer, kidney cancer, gastrointestinal cancer, urothelial cancer, pharynx cancer, head and neck cancer, rectal cancer, esophagus cancer, or bladder cancer, or a metastasis thereof,
(vi) an autoimmune disease, optionally wherein the viral vector or nucleic acid encodes a CAR that binds to a B cell antigen, e.g., CD 19, CD20, CD22, CD123, FcRn5, FcRn2, BCMA, CS-1 and CD138.

52. A kit comprising the first composition and the second composition of the plurality of compositions of any one of claims 1, 5-18, 20-21, or 24-39.

53. The plurality of compositions of any one of claims 1, 5-18, 20-21, or 24-39, the method of any one of claims 3-18, 20-37, 40-46, or 50-51, or the second composition of any one of claims 19-21, 24-27, 29-36, or 38, wherein the viral vector or the nucleic acid encodes:
(i) a first CAR that binds to a B cell antigen and a second CAR that binds to (a) a solid tumor antigen, (b) a myeloid tumor antigen, or (c) an antigen of a hematological tumor not of B-cell lineage; or
(2) a CAR that comprises a first binding domain that binds to a B cell antigen and a second binding domain that binds to (a) a solid tumor antigen, (b) a myeloid tumor antigen, or (c) an antigen of a hematological tumor not of B-cell lineage.

54. The method of claim 53, wherein the disease, disorder, or condition is a solid tumor.
